SEQUENCE LISTING (30 sequences)

SEQ ID NO: 1 - DNA, 90 bp, Hepatitis C virus
ggcctacttggctgcatcatcactagcctcacaggccgagacaggaaccaggtcgagggggaggtccaagtggtctccaccgcaacacaa

SEQ ID NO: 2 - DNA, 90 bp, Hepatitis C virus
ggcctacttggctgcatcatcactagcctcacaggccgagacaggaaccaggtcgagggggaggtccaagtggtctccaccgtaacacaa

SEQ ID NO: 3 - DNA, 90 bp, Hepatitis C virus; identical to SEQ ID NO: 2

SEQ ID NO: 4 - DNA, 90 bp, Hepatitis C virus; identical to SEQ ID NO: 2

SEQ ID NO: 5 - DNA, 90 bp, Hepatitis C virus; identical to SEQ ID NO: 2

SEQ ID NO: 6 - DNA, 90 bp, Hepatitis C virus
ggcctacttggcagcattatcactagcctcacaggccgagacaggaaccaagtcgagggggaggtccaagtggtctccaccgcaacacaa

SEQ ID NO: 7 - DNA, 90 bp, Hepatitis C virus
ggcttacttggcagcatcatcactagcctcacaggccgagacaggaaccaggtcgagggggaggtccaagtggtctccaccgcaacacaa

SEQ ID NO: 8 - DNA, 8001 bp, Hepatitis C virus
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaacgggcgcgccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttaaacagaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatggaaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaaccggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggaaggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcgtaggctggccaggggatctcccccctccttggccagctcatcagctagccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacggggagctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagt

SEQ ID NO: 9 - DNA, 8649 bp, Hepatitis C virus
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaacgggcgcgccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttaaacagaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatggaccgggagatggcagcatcgtgcggaggcgcggttttcgtaggtctgatactcttgaccttgtcaccgcactataagctgttcctcgctaggctcatatggtggttacaatattttatcaccagggccgaggcacacttgcaagtgtggatcccccccctcaacgttcgggggggccgcgatgccgtcatcctcctcacgtgcgcgatccacccagagctaatctttaccatcaccaaaatcttgctcgccatactcggtccactcatggtgctccaggctggtataaccaaagtgccgtacttcgtgcgcgcacacgggctcattcgtgcatgcatgctggtgcggaaggttgctgggggtcattatgtccaaatggctctcatgaagttggccgcactgacaggtacgtacgtttatgaccatctcaccccactgcgggactgggcccacgcgggcctacgagaccttgcggtggcagttgagcccgtcgtcttctctgatatggagaccaaggttatcacctggggggcagacaccgcggcgtgtggggacatcatcttgggcctgcccgtctccgcccgcagggggagggagatacatctgggaccggcagacagccttgaagggcaggggtggcgactcctcgcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatggaaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaaccggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggaaggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcgtaggctggccaggggatctcccccctccttggccagctcatcagctagccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacggggagctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagt

SEQ ID NO: 10 - DNA, 7989 bp, Hepatitis C virus
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaagggcgcgccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttaaacagaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatggaaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaaccggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggaaggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcgtaggctggccaggggatctcccccctccttggccagctcatcagctagccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacggggagctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagt

SEQ ID NO: 11 - DNA, 8637 bp, Hepatitis C virus
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaagggcgcgccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttaaacagaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatggaccgggagatggcagcatcgtgcggaggcgcggttttcgtaggtctgatactcttgaccttgtcaccgcactataagctgttcctcgctaggctcatatggtggttacaatattttatcaccagggccgaggcacacttgcaagtgtggatcccccccctcaacgttcgggggggccgcgatgccgtcatcctcctcacgtgcgcgatccacccagagctaatctttaccatcaccaaaatcttgctcgccatactcggtccactcatggtgctccaggctggtataaccaaagtgccgtacttcgtgcgcgcacacgggctcattcgtgcatgcatgctggtgcggaaggttgctgggggtcattatgtccaaatggctctcatgaagttggccgcactgacaggtacgtacgtttatgaccatctcaccccactgcgggactgggcccacgcgggcctacgagaccttgcggtggcagttgagcccgtcgtcttctctgatatggagaccaaggttatcacctggggggcagacaccgcggcgtgtggggacatcatcttgggcctgcccgtctccgcccgcagggggagggagatacatctgggaccggcagacagccttgaagggcaggggtggcgactcctcgcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatggaaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaaccggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggaaggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcgtaggctggccaggggatctcccccctccttggccagctcatcagctagccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacggggagctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagt

SEQ ID NO: 12 - DNA, 9646 bp, Hepatitis C virus
gccagccccctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggataaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgtcgcccacaggacgtcaagttcccgggtggcggtcagatcgttggtggagtttacttgttgccgcgcaggggccctagattgggtgtgcgcgcgacgaggaagacttccgagcggtcgcaacctcgaggtagacgtcagcctatccccaaggcacgtcggcccgagggcaggacctgggctcagcccgggtacccttggcccctctatggcaatgagggttgcgggtgggcgggatggctcctgtctccccgtggctctcggcctagctggggccccacagacccccggcgtaggtcgcgcaatttgggtaaggtcatcgatacccttacgtgcggcttcgccgacctcatggggtacataccgctcgtcggcgcccctcttggaggcgctgccagggccctggcgcatggcgtccgggttctggaagacggcgtgaactatgcaacagggaaccttcctggttgctctttctctatcttccttctggccctgctctcttgcctgactgtgcccgcttcagcctaccaagtgcgcaattcctcggggctttaccatgtcaccaatgattgccctaactcgagtattgtgtacgaggcggccgatgccatcctgcacactccggggtgtgtcccttgcgttcgcgagggtaacgcctcgaggtgttgggtggcggtgacccccacggtggccaccagggacggcaaactccccacaacgcagcttcgacgtcatatcgatctgcttgtcgggagcgccaccctctgctcggccctctacgtgggggacctgtgcgggtctgtctttcttgttggtcaactgtttaccttctctcccaggcgccactggacgacgcaagactgcaattgttctatctatcccggccatataacgggtcatcgcatggcatgggatatgatgatgaactggtcccctacggcagcgttggtggtagctcagctgctccggatcccacaagccatcatggacatgatcgctggtgctcactggggagtcctggcgggcatagcgtatttctccatggtggggaactgggcgaaggtcctggtagtgctgctgctatttgccggcgtcgacgcggaaacccacgtcaccgggggaagtgccggccgcaccacggctgggcttgttggtctccttacaccaggcgccaagcagaacatccaactgatcaacaccaacggcagttggcacatcaatagcacggccttgaactgcaatgaaagccttaacaccggctggttagcagggctcttctatcagcacaaattcaactcttcaggctgtcctgagaggttggccagctgccgacgccttaccgattttgcccagggctggggtcctatcagttatgccaacggaagcggcctcgacgaacgcccctactgctggcactaccctccaagaccttgtggcattgtgcccgcaaagagcgtgtgtggcccggtatattgcttcactcccagccccgtggtggtgggaacgaccgacaggtcgggcgcgcctacctacagctggggtgcaaatgatacggatgtcttcgtccttaacaacaccaggccaccgctgggcaattggttcggttgtacctggatgaactcaactggattcaccaaagtgtgcggagcgcccccttgtgtcatcggaggggtgggcaacaacaccttgctctgccccactgattgtttccgcaagcatccggaagccacatactctcggtgcggctccggtccctggattacacccaggtgcatggtcgactacccgtataggctttggcactatccttgtaccatcaattacaccatattcaaagtcaggatgtacgtgggaggggtcgagcacaggctggaagcggcctgcaactggacgcggggcgaacgctgtgatctggaagacagggacaggtccgagctcagcccattgctgctgtccaccacacagtggcaggtccttccgtgttctttcacgaccctgccagccttgtccaccggcctcatccacctccaccagaacattgtggacgtgcagtacttgtacggggtagggtcaagcatcgcgtcctgggccattaagtgggagtacgtcgttctcctgttcctcctgcttgcagacgcgcgcgtctgctcctgcttgtggatgatgttactcatatcccaagcggaggcggctttggagaacctcgtaatactcaatgcagcatccctggccgggacgcacggtcttgtgtccttcctcgtgttcttctgctttgcgtggtatctgaagggtaggtgggtgcccggagcggtctacgccttctacgggatgtggcctctcctcctgctcctgctggcgttgcctcagcgggcatacgcactggacacggaggtggccgcgtcgtgtggcggcgttgttcttgtcgggttaatggcgctgactctgtcgccatattacaagcgctacatcagctggtgcatgtggtggcttcagtattttctgaccagagtagaagcgcaactgcacgtgtgggttccccccctcaacgtccggggggggcgcgatgccgtcatcttactcatgtgtgttgtacacccgactctggtatttgacatcaccaaactactcctggccatcttcggacccctttggattcttcaagccagtttgcttaaagtcccctacttcgtgcgcgttcaaggccttctccggatctgcgcgctagcgcggaagatagccggaggtcattacgtgcaaatggccatcatcaagttaggggcgcttactggcacctatgtgtataaccatctcacccctcttcgagactgggcgcacaacggcctgcgagatctggccgtggctgtggaaccagtcgtcttctcccgaatggagaccaagctcatcacgtggggggcagataccgccgcgtgcggtgacatcatcaacggcttgcccgtctctgcccgtaggggccaggagatactgcttgggccagccgacggaatggtctccaaggggtggaggttgctggcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctggactgtctaccacggggccggaacgaggaccatcgcatcacccaagggtcctgtcatccagatgtataccaatgtggaccaagaccttgtgggctggcccgctcctcaaggttcccgctcattgacaccctgcacctgcggctcctcggacctttacctggtcacgaggcacgccgatgtcattcccgtgcgccggcgaggtgatagcaggggtagcctgctttcgccccggcccatttcctacttgaaaggctcctcggggggtccgctgttgtgccccgcgggacacgccgtgggcctattcagggccgcggtgtgcacccgtggagtggctaaggcggtggactttatccctgtggagaacctagagacaaccatgagatccccggtgttcacggacaactcctctccaccagcagtgccccagagcttccaggtggcccacctgcatgctcccaccggcagcggtaagagcaccaaggtcccggctgcgtacgcagcccagggctacaaggtgttggtgctcaacccctctgttgctgcaacgctgggctttggtgcttacatgtccaaggcccatggggttgatcctaatatcaggaccggggtgagaacaattaccactggcagccccatcacgtactccacctacggcaagttccttgccgacggcgggtgctcaggaggtgcttatgacataataatttgtgacgagtgccactccacggatgccacatccatcttgggcatcggcactgtccttgaccaagcagagactgcgggggcgagactggttgtgctcgccactgctacccctccgggctccgtcactgtgtcccatcctaacatcgaggaggttgctctgtccaccaccggagagatccctttttacggcaaggctatccccctcgaggtgatcaaggggggaagacatctcatcttctgccactcaaagaagaagtgcgacgagctcgccgcgaagctggtcgcattgggcatcaatgccgtggcctactaccgcggtcttgacgtgtctgtcatcccgaccagcggcgatgttgtcgtcgtgtcgaccgatgctctcatgactggctttaccggcgacttcgactctgtgatagactgcaacacgtgtgtcactcagacagtcgatttcagccttgaccctacctttaccattgagacaaccacgctcccccaggatgctgtctccaggactcaacgccggggcaggactggcagggggaagccaggcatctacagatttgtggcaccgggggagcgcccctccggcatgttcgactcgtccgtcctctgtgagtgctatgacgcgggctgtgcttggtatgagctcacgcccgccgagactacagttaggctacgagcgtacatgaacaccccggggcttcccgtgtgccaggaccatcttgaattttgggagggcgtctttacgggcctcactcatatagatgcccactttctatcccagacaaagcagagtggggagaactttccttacctggtagcgtaccaagccaccgtgtgcgctagggctcaagcccctcccccatcgtgggaccagatgtggaagtgtttgatccgccttaaacccaccctccatgggccaacacccctgctatacagactgggcgctgttcagaatgaagtcaccctgacgcacccaatcaccaaatacatcatgacatgcatgtcggccgacctggaggtcgtcacgagcacctgggtgctcgttggcggcgtcctggctgctctggccgcgtattgcctgtcaacaggctgcgtggtcatagtgggcaggattgtcttgtccgggaagccggcaattatacctgacagggaggttctctaccaggagttcgatgagatggaagagtgctctcagcacttaccgtacatcgagcaagggatgatgctcgctgagcagttcaagcagaaggccctcggcctcctgcagaccgcgtcccgccaagcagaggttatcacccctgctgtccagaccaactggcagaaactcgaggtcttctgggcgaagcacatgtggaatttcatcagtgggatacaatacttggcgggcctgtcaacgctgcctggtaaccccgccattgcttcattgatggcttttacagctgccgtcaccagcccactaaccactggccaaaccctcctcttcaacatattgggggggtgggtggctgcccagctcgccgcccccggtgccgctaccgcctttgtgggcgctggcttagctggcgccgccatcggcagcgttggactggggaaggtcctcgtggacattcttgcagggtatggcgcgggcgtggcgggagctcttgtagcattcaagatcatgagcggtgaggtcccctccacggaggacctggtcaatctgctgcccgccatcctctcgcctggagcccttgtagtcggtgtggtctgcgcagcaatactgcgccggcacgttggcccgggcgagggggcagtgcaatggatgaaccggctaatagccttcgcctcccgggggaaccatgtttcccccacgcactacgtgccggagagcgatgcagccgcccgcgtcactgccatactcagcagcctcactgtaacccagctcctgaggcgactgcatcagtggataagctcggagtgtaccactccatgctccggttcctggctaagggacatctgggactggatatgcgaggtgctgagcgactttaagacctggctgaaagccaagctcatgccacaactgcctgggattccctttgtgtcctgccagcgcgggtataggggggtctggcgaggagacggcattatgcacactcgctgccactgtggagctgagatcactggacatgtcaaaaacgggacgatgaggatcgtcggtcctaggacctgcaggaacatgtggagtgggacgttccccattaacgcctacaccacgggcccctgtactccccttcctgcgccgaactataagttcgcgctgtggagggtgtctgcagaggaatacgtggagataaggcgggtgggggacttccactacgtatcgggtatgactactgacaatcttaaatgcccgtgccagatcccatcgcccgaatttttcacagaattggacggggtgcgcctacataggtttgcgcccccttgcaagcccttgctgcgggaggaggtatcattcagagtaggactccacgagtacccggtggggtcgcaattaccttgcgagcccgaaccggacgtagccgtgttgacgtccatgctcactgatccctcccatataacagcagaggcggccgggagaaggttggcgagagggtcacccccttctatggccagctcctcggccagccagctgtccgctccatctctcaaggcaacttgcaccgccaaccatgactcccctgacgccgagctcatagaggctaacctcctgtggaggcaggagatgggcggcaacatcaccagggttgagtcagagaacaaagtggtgattctggactccttcgatccgcttgtggcagaggaggatgagcgggaggtctccgtacccgcagaaattctgcggaagtctcggagattcgcccgggccctgcccgtttgggcgcggccggactacaaccccccgctagtagagacgtggaaaaagcctgactacgaaccacctgtggtccatggctgcccgctaccacctccacggtcccctcctgtgcctccgcctcggaaaaagcgtacggtggtcctcaccgaatcaaccctatctactgccttggccgagcttgccaccaaaagttttggcagctcctcaacttccggcattacgggcgacaatacgacaacatcctctgagcccgccccttctggctgcccccccgactccgacgttgagtcctattcttccatgccccccctggagggggagcctggggatccggatctcagcgacgggtcatggtcgacggtcagtagtggggccgacacggaagatgtcgtgtgctgctcaatgtcttattcctggacaggcgcactcgtcaccccgtgcgctgcggaagaacaaaaactgcccatcaacgcactgagcaactcgttgctacgccatcacaatctggtgtattccaccacttcacgcagtgcttgccaaaggcagaagaaagtcacatttgacagactgcaagttctggacagccattaccaggacgtgctcaaggaggtcaaagcagcggcgtcaaaagtgaaggctaacttgctatccgtagaggaagcttgcagcctgacgcccccacattcagccaaatccaagtttggctatggggcaaaagacgtccgttgccatgccagaaaggccgtagcccacatcaactccgtgtggaaagaccttctggaagacagtgtaacaccaatagacactaccatcatggccaagaacgaggttttctgcgttcagcctgagaaggggggtcgtaagccagctcgtctcatcgtgttccccgacctgggcgtgcgcgtgtgcgagaagatggccctgtacgacgtggttagcaagctccccctggccgtgatgggaagctcctacggattccaatactcaccaggacagcgggttgaattcctcgtgcaagcgtggaagtccaagaagaccccgatggggttctcgtatgatacccgctgttttgactccacagtcactgagagcgacatccgtacggaggaggcaatttaccaatgttgtgacctggacccccaagcccgcgtggccatcaagtccctcactgagaggctttatgttgggggccctcttaccaattcaaggggggaaaactgcggctaccgcaggtgccgcgcgagcggcgtactgacaactagctgtggtaacaccctcacttgctacatcaaggcccgggcagcctgtcgagccgcagggctccaggactgcaccatgctcgtgtgtggcgacgacttagtcgttatctgtgaaagtgcgggggtccaggaggacgcggcgagcctgagagccttcacggaggctatgaccaggtactccgccccccccggggaccccccacaaccagaatacgacttggagcttataacatcatgctcctccaacgtgtcagtcgcccacgacggcgctggaaagagggtctactaccttacccgtgaccctacaacccccctcgcgagagccgcgtgggagacagcaagacacactccagtcaattcctggctaggcaacataatcatgtttgcccccacactgtgggcgaggatgatactgatgacccatttctttagcgtcctcatagccagggatcagcttgaacaggctcttaactgtgagatctacggagcctgctactccatagaaccactggatctacctccaatcattcaaagactccatggcctcagcgcattttcactccacagttactctccaggtgaaatcaatagggtggccgcatgcctcagaaaacttggggtcccgcccttgcgagcttggagacaccgggcccggagcgtccgcgctaggcttctgtccagaggaggcagggctgccatatgtggcaagtacctcttcaactgggcagtaagaacaaagctcaaactcactccaatagcggccgctggccggctggacttgtccggttggttcacggctggctacagcgggggagacatttatcacagcgtgtctcatgcccggccccgctggttctggttttgcctactcctgctcgctgcaggggtaggcatctacctcctccccaaccgatgaaggttggggtaaacactccggcctcttaggccatttcctgtttttttttttttttttttttttttttttttttttttttttttttttttttctttttttttttttttttccttttttttttttttttttttctttccttcttttttcctttcttttccttccttctttaatggtggctccatcttagccctagtcacggctagctgtgaaaggtccgtgagccgcatgactgcagagagtgctgatactggcctctctgcagatcatgt

SEQ ID NO: 13 - DNA, 9599 bp, Hepatitis C virus
gccagccccctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggataaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgtcgcccacaggacgtcaagttcccgggtggcggtcagatcgttggtggagtttacttgttgccgcgcaggggccctagattgggtgtgcgcgcgacgaggaagacttccgagcggtcgcaacctcgaggtagacgtcagcctatccccaaggcacgtcggcccgagggcaggacctgggctcagcccgggtacccttggcccctctatggcaatgagggttgcgggtgggcgggatggctcctgtctccccgtggctctcggcctagctggggccccacagacccccggcgtaggtcgcgcaatttgggtaaggtcatcgatacccttacgtgcggcttcgccgacctcatggggtacataccgctcgtcggcgcccctcttggaggcgctgccagggccctggcgcatggcgtccgggttctggaagacggcgtgaactatgcaacagggaaccttcctggttgctctttctctatcttccttctggccctgctctcttgcctgactgtgcccgcttcagcctaccaagtgcgcaattcctcggggctttaccatgtcaccaatgattgccctaactcgagtattgtgtacgaggcggccgatgccatcctgcacactccggggtgtgtcccttgcgttcgcgagggtaacgcctcgaggtgttgggtggcggtgacccccacggtggccaccagggacggcaaactccccacaacgcagcttcgacgtcatatcgatctgcttgtcgggagcgccaccctctgctcggccctctacgtgggggacctgtgcgggtctgtctttcttgttggtcaactgtttaccttctctcccaggcgccactggacgacgcaagactgcaattgttctatctatcccggccatataacgggtcatcgcatggcatgggatatgatgatgaactggtcccctacggcagcgttggtggtagctcagctgctccggatcccacaagccatcatggacatgatcgctggtgctcactggggagtcctggcgggcatagcgtatttctccatggtggggaactgggcgaaggtcctggtagtgctgctgctatttgccggcgtcgacgcggaaacccacgtcaccgggggaaatgccggccgcaccacggctgggcttgttggtctccttacaccaggcgccaagcagaacatccaactgatcaacaccaacggcagttggcacatcaatagcacggccttgaattgcaatgaaagccttaacaccggctggttagcagggctcttctatcaacacaaattcaactcttcaggctgtcctgagaggttggccagctgccgacgccttaccgattttgcccagggctggggtcctatcagttatgccaacggaagcggcctcgacgaacgcccctactgctggcactaccctccaagaccttgtggcattgtgcccgcaaagagcgtgtgtggcccggtatattgcttcactcccagccccgtggtggtgggaacgaccgacaggtcgggcgcgcctacctacagctggggtgcaaatgatacggatgtcttcgtccttaacaacaccaggccaccgctgggcaattggttcggttgtacctggatgaactcaactggattcaccaaagtgtgcggagcgcccccttgtgtcatcggaggggtgggcaacaacaccttgctctgccccactgattgcttccgcaaacatccggaagccacatactctcggtgcggctccggtccctggattacacccaggtgcatggtcgactacccgtataggctttggcactatccttgtaccatcaattacaccatattcaaagtcaggatgtacgtgggaggggtcgagcacaggctggaagcggcctgcaactggacgcggggcgaacgctgtgatctggaagacagggacaggtccgagctcagcccgttgctgctgtccaccacacagtggcaggtccttccgtgttctttcacgaccctgccagccttgtccaccggcctcatccacctccaccagaacattgtggacgtgcagtacttgtacggggtagggtcaagcatcgcgtcctgggccattaagtgggagtacgtcgttctcctgttccttctgcttgcagacgcgcgcgtctgctcctgcttgtggatgatgttactcatatcccaagcggaggcggctttggagaacctcgtaatactcaatgcagcatccctggccgggacgcacggtcttgtgtccttcctcgtgttcttctgctttgcgtggtatctgaagggtaggtgggtgcccggagcggtctacgccctctacgggatgtggcctctcctcctgctcctgctggcgttgcctcagcgggcatacgcactggacacggaggtggccgcgtcgtgtggcggcgttgttcttgtcgggttaatggcgctgactctgtcgccatattacaagcgctatatcagctggtgcatgtggtggcttcagtattttctgaccagagtagaagcgcaactgcacgtgtgggttccccccctcaacgtccggggggggcgcgatgccgtcatcttactcatgtgtgtagtacacccgaccctggtatttgacatcaccaaactactcctggccatcttcggacccctttggattcttcaagccagtttgcttaaagtcccctacttcgtgcgcgttcaaggccttctccggatctgcgcgctagcgcggaagatagccggaggtcattacgtgcaaatggccatcatcaagttaggggcgcttactggcacctatgtgtataaccatctcacccctcttcgagactgggcgcacaacggcctgcgagatctggccgtggctgtggaaccagtcgtcttctcccgaatggagaccaagctcatcacgtggggggcagataccgccgcgtgcggtgacatcatcaacggcttgcccgtctctgcccgtaggggccaggagatactgcttgggccagccgacggaatggtctccaaggggtggaggttgctggcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctggactgtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatagcaggggtagcctgctttcgccccggcccatttcctacttgaaaggctcctcggggggtccgctgttgtgccccgcgggacacgccgtgggcctattcagggccgcggtgtgcacccgtggagtggctaaagcggtggactttatccctgtggagaacctagggacaaccatgagatccccggtgttcacggacaactcctctccaccagcagtgccccagagcttccaggtggcccacctgcatgctcccaccggcagcggtaagagcaccaaggtcccggctgcgtacgcagcccagggctacaaggtgttggtgctcaacccctctgttgctgcaacgctgggctttggtgcttacatgtccaaggcccatggggttgatcctaatatcaggaccggggtgagaacaattaccactggcagccccatcacgtactccacctacggcaagttccttgccgacggcgggtgctcaggaggtgcttatgacataataatttgtgacgagtgccactccacggatgccacatccatcttgggcatcggcactgtccttgaccaagcagagactgcgggggcgagactggttgtgctcgccactgctacccctccgggctccgtcactgtgtcccatcctaacatcgaggaggttgctctgtccaccaccggagagatccccttttacggcaaggctatccccctcgaggtgatcaaggggggaagacatctcatcttctgccactcaaagaagaagtgcgacgagctcgccgcgaagctggtcgcattgggcatcaatgccgtggcctactaccgcggtcttgacgtgtctgtcatcccgaccagcggcgatgttgtcgtcgtgtcgaccgatgctctcatgactggctttaccggcgacttcgactctgtgatagactgcaacacgtgtgtcactcagacagtcgatttcagccttgaccctacctttaccattgagacaaccacgctcccccaggatgctgtctccaggactcaacgccggggcaggactggcagggggaagccaggcatctatagatttgtggcaccgggggagcgcccctccggcatgttcgactcgtccgtcctctgtgagtgctatgacgcgggctgtgcttggtatgagctcacgcccgccgagactacagttaggctacgagcgtacatgaacaccccggggcttcccgtgtgccaggaccatcttgaattttgggagggcgtctttacgggcctcactcatatagatgcccactttttatcccagacaaagcagagtggggagaactttccttacctggtagcgtaccaagccaccgtgtgcgctagggctcaagcccctcccccatcgtgggaccagatgtggaagtgtttgatccgccttaaacccaccctccatgggccaacacccctgctatacagactgggcgctgttcagaatgaagtcaccctgacgcacccaatcaccaaatacatcatgacatgcatgtcggccgacctggaggtcgtcacgagcacctgggtgctcgttggcggcgtcctggctgctctggccgcgtattgcctgtcaacaggctgcgtggtcatagtgggcaggatcgtcttgtccgggaagccggcaattatacctgacagggaggttctctaccaggagttcgatgagatggaagagtgctctcagcacttaccgtacatcgagcaagggatgatgctcgctgagcagttcaagcagaaggccctcggcctcctgcagaccgcgtcccgccatgcagaggttatcacccctgctgtccagaccaactggcagaaactcgaggtcttttgggcgaagcacatgtggaatttcatcagtgggatacaatacttggcgggcctgtcaacgctgcctggtaaccccgccattgcttcattgatggcttttacagctgccgtcaccagcccactaaccactggccaaaccctcctcttcaacatattgggggggtgggtggctgcccagctcgccgcccccggtgccgctactgcctttgtgggtgctggcctagctggcgccgccatcggcagcgttggactggggaaggtcctcgtggacattcttgcagggtatggcgcgggcgtggcgggagctcttgtagcattcaagatcatgagcggtgaggtcccctccacggaggacctggtcaatctgctgcccgccatcctctcgcctggagcccttgtagtcggtgtggtctgcgcagcaatactgcgccggcacgttggcccgggcgagggggcagtgcaatggatgaaccggctaatagccttcgcctcccgggggaaccatgtttcccccacgcactacgtgccggagagcgatgcagccgcccgcgtcactgccatactcagcagcctcactgtaacccagctcctgaggcgactgcatcagtggataagctcggagtgtaccactccatgctccggttcctggctaagggacatctgggactggatatgcgaggtgctgagcgactttaagacctggctgaaagccaagctcatgccacaactgcctgggattccctttgtgtcctgccagcgcgggtataggggggtctggcgaggagacggcattatgcacactcgctgccactgtggagctgagatcactggacatgtcaaaaacgggacgatgaggatcgtcggtcctaggacctgcaggaacatgtggagtgggacgttccccattaacgcctacaccacgggcccctgtactccccttcctgcgccgaactataagttcgcgctgtggagggtgtctgcagaggaatacgtggagataaggcgggtgggggacttccactacgtatcgggtatgactactgacaatcttaaatgcccgtgccagatcccatcgcccgaatttttcacagaattggacggggtgcgcctacacaggtttgcgcccccttgcaagcccttgctgcgggaggaggtatcattcagagtaggactccacgagtacccggtggggtcgcaattaccttgcgagcccgaaccggacgtagccgtgttgacgtccatgctcactgatccctcccatataacagcagaggcggccgggagaaggttggcgagagggtcacccccttctatggccagctcctcggctagccagctgtccgctccatctctcaaggcaacttgcaccgccaaccatgactcccctgacgccgagctcatagaggctaacctcctgtggaggcaggagatgggcggcaacatcaccagggttgagtcagagaacaaagtggtgattctggactccttcgatccgcttgtggcagaggaggatgagcgggaggtctccgtacctgcagaaattctgcggaagtctcggagattcgcccgggccctgcccgtctgggcgcggccggactacaaccccccgctagtagagacgtggaaaaagcctgactacgaaccacctgtggtccatggctgcccgctaccacctccacggtcccctcctgtgcctccgcctcggaaaaagcgtacggtggtcctcaccgaatcaaccctatctactgccttggccgagcttgccaccaaaagttttggcagctcctcaacttccggcattacgggcgacaatacgacaacatcctctgagcccgccccttctggctgcccccccgactccgacgttgagtcctattcttccatgccccccctggagggggagcctggggatccggatctcagcgacgggtcatggtcgacggtcagtagtggggccgacacggaagatgtcgtgtgctgctcaatgtcttattcctggacaggcgcactcgtcaccccgtgcgctgcggaagaacaaaaactgcccatcaacgcactgagcaactcgttgctacgccatcacaatctggtgtattccaccacttcacgcagtgcttgccaaaggcagaagaaagtcacatttgacagactgcaagttctggacagccattaccaggacgtgctcaaggaggtcaaagcagcggcgtcaaaagtgaaggctaacttgctatccgtagaggaagcttgcagcctgacgcccccacattcagccaaatccaagtttggctatggggcaaaagacgtccgttgccatgccagaaaggccgtagcccacatcaactccgtgtggaaagaccttctggaagacagtgtaacaccaatagacactaccatcatggccaagaacgaggttttctgcgttcagcctgagaaggggggtcgtaagccagctcgtctcatcgtgttccccgacctgggcgtgcgcgtgtgcgagaagatggccctgtacgacgtggttagcaagctccccctggccgtgatgggaagctcctacggattccaatactcaccaggacagcgggttgaattcctcgtgcaagcgtggaagtccaagaagaccccgatggggttctcgtatgatacccgctgttttgactccacagtcactgagagcgacatccgtacggaggaggcaatttaccaatgttgtgacctggacccccaagcccgcgtggccatcaagtccctcactgagaggctttatgttgggggccctcttaccaattcaaggggggaaaactgcggctaccgcaggtgccgcgcgagcggcgtactgacaactagctgtggtaacaccctcacttgctacatcaaggcccgggcagcctgtcgagccgcagggctccaggactgcaccatgctcgtgtgtggcgacgacttagtcgttatctgtgaaagtgcgggggtccaggaggacgcggcgagcctgagagccttcacggaggctatgaccaggtactccgccccccccggggaccccccacaaccagaatacgacttggagcttataacatcatgctcctccaacgtgtcagtcgcccacgacggcgctggaaagagggtctactaccttacccgtgaccctacaacccccctcgcgagagccgcgtgggagacagcaagacacactccagtcaattcctggctaggcaacataatcatgtttgcccccacactgtgggcgaggatgatactgatgacccatttctttagcgtcctcatagccagggatcagcttgaacaggctcttaactgtgagatctacggagcctgctactccatagaaccactggatctacctccaatcattcaaagactccatggcctcagcgcattttcactccacagttactctccaggtgaaatcaatagggtggccgcatgcctcagaaaacttggggtcccgcccttgcgagcttggagacaccgggcccggagcgtccgcgctaggcttctgtccagaggaggcagggctgccatatgtggcaagtacctcttcaactgggcagtaagaacaaagctcaaactcactccaatagcggccgctggccggctggacttgtccggttggttcacggctggctacagcgggggagacatttatcacagcgtgtctcatgcccggccccgctggttctggttttgcctactcctgctcgctgcaggggtaggcatctacctcctccccaaccgatgaaggttggggtaaacactccggcctcttaagccatttcctgttttttttttttttttttttttttttttctttttttttttctttcctttccttctttttttcctttctttttcccttctttaatggtggctccatcttagccctagtcacggctagctgtgaaaggtccgtgagccgcatgactgcagagagtgctgatactggcctctctgcagatcatgt

SEQ ID NO: 14 - DNA, 9616 bp, Hepatitis C virus
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccgcccacaggacgtcaagttcccgggcggtggtcagatcgttggtggagtttacctgttgccgcgcaggggccccaggttgggtgtgcgcgcgatcaggaagacttccgagcggtcgcaaccccgtggaaggcgacagcctatccccaaggctcgccggcccgagggcagggcctgggctcagcccgggtatccttggcccctctatggcaatgagggcatggggtgggcaggatggctcctgtcaccccgcggctcccggcctagttggggccccacggacccccggcgtaggtcgcgtaatttgggtaaggtcatcgataccctcacatgcggcctcgccgacctcatggggtacattccgctcgtcggcggccccctagggggcgctgccagggccttggcacatggtgtccgggttctggaggacggcgtgaactatgcaacagggaacctgcccggttgctctttttctatcttcctcttggctctgctgtcctgtctgaccgtaccagcttccgctcatgaagtgcgtaacgcgtccggggtataccatgtcacgaacgactgctccaactcaagcattgtgtttgaggcggcggacttgatcatgcatactcccgggtgcgtgccctgcgttcgggagggtaactcctcccgctgctgggtagcgctcactcccacgctcgcggccaggaatgctaccatccccactacgacaatacgacaccacgtcgatttgctcgttggggcggctgctctctgctccgctatgtacgtgggggacctctgcggatctgttttcctcgtctctcagctgttcaccttctcgccccgccggcatgcgacattgcaggactgcaattgttcgatctaccccggccacgcgtcaggtcaccgcatggcctgggacatgatgatgaactggtcacctacaacagccctcgtagtgtcgcagttactccggatcccacaagccgtcatcgacatggtggcgggggcccactggggagtcctggcgggccttgcctactattccatggcggggaactgggctaaggttttgattgtgatgctactttttgccggcgttgacgggcacaccctcacaacgggggggcacgctgcccgcctcaccagcgggttcgcgggcctctttacacctgggccgtctcagagaatccagcttataaacaccaatggcagttggcacatcaacaggactgccctgaactgcaatgactccctccagactgggtttcttgccgcgctgttctacgcacataggttcaactcgtccggatgcccggagcgcatggccagctgccgctccattgacaagttcgaccagggatggggtcctatcacttatgctgagcctacaaaagacccggaccagaggccttattgctggcactacccacctcaacaatgtggtatcgtacctgcgtcgcaggtgtgtggtccagtgtattgcttcaccccaagtcctgttgtcgtggggacaaccgatcgtctcggcaaccctacgtacagctggggggagaacgatactgacgtgctgctccttaacaacacgcggccgccgcaaggcaactggttcggctgtacatggatgaatagcactgggttcaccaagacgtgcggggcccccccgtgtaacatcgggggggtcggcaataacaccttgacctgccccacggactgcttccggaagcaccccgaggccacgtactcaaaatgtggctcggggccttggttgacacctaggtgcatggttgactacccatacaggctctggcactacccctgcactgtcaacttctccatctttaaggttaggatgtatgtggggggcgtggagcacaggcttaatgctgcatgcaactggacccgaggagagcgttgcaacttggacgacagggacagatcggagctcagcccgctgctgctctctacaacagagtggcaggttctgccctgctctttcaccaccctaccggctctgtccactggcttgatccacctccatcagaacatcgtggacgtgcaatacctgtacggtatagggtcagcggttgtctcctttgcaatcaaatgggagtatgtcgtgttgcttttccttctcctggcggacgcgcgcgtctgtgcctgcttgtggatgatgctgctgatagcccaggccgaggccgccttagagaacctggtggccctcaatgcagcgtccgttgccggagcgcacggcatcctctccttcctcgtgttcttctgtgccgcttggtacatcaagggcaggctggtccctggggcggcatatgctttctatggcgcatggccgctgctcctgctcctcttgacattaccaccacgagcttacgccatggaccgggagatggctgcatcgtgcggaggcgcggtttttgtgggtctggcattattgaccttgtcgccatattacaaggtgttcctcgctaggctcctatggtggttacaatatcttatcaccagagctgaggcgcacttgcatgtgtgggttccccccctcaacgtccggggaggccgcgatgccatcatcctcctcacgtgtgcagtccacccagagctaatctttgatatcaccaaacttctgattgccatactcggaccgctcatggtgctccaagctggcataactagggtgccgtacttcgtacgcgctcaagggctcattcgtgcatgcatgttagtgcggaaagtcgctgggggtcattatgtccaaatggccttcatgagactgggcgcgctgacgggcacgtacgtctataatcacctcaccccactgcgggattgggcccacgccggcctacgggaccttgcggtagcagtggagcctgtcgtcttctctgacatggagaccaagatcatcacctggggggcagacaccgcggcgtgtggggacatcatcctgggcctacctgtctccgcccgaaggggaagggagatactcctggggccggccgatagtctagtagggcaggggtggcgactccttgcgcccatcacggcctactcccaacagacccggggcctacttggttgcatcatcacgagtctcacaggccgggacaagaaccaggtcgagggggaggttcaagtggtctccaccgcaacacaatctttcctggcgacctgcgtcaacggcgtatgttggactgtctaccatggtgctggctcaaagactctagccggcccaaaaggcccaatcgcccagatgtacactaatgtagaccaggatctcgtcggctggccggcgccccccggggcgcgttccctgacaccatgcacctgtggcagctcggacctttacttggttacgagacatgcagatgttattccggtgcgccggcggggcgacaatagagggagcttgctctcccccaggcctgtctcctacttgaagggctcttcgggtggcccactgctctgcccttcggggcacgctgtgggcgtcttccgggccgctgtatgcacccggggggttgcaaaggcggtggattttgtccccgttgagtccatggaaactactatgcggtccccggtcttcacagacaactcatctcccccggccgtaccgcaaacattccaagtggcccatctacacgctcccactggcagcggcaagagcactagagtgccggccgcatatgcggcccaagggtacaaggtgcttgtcctgaacccgtctgttgccgctaccttaggttttggggcgtatatgtctaaagcacatggtaccgaccctaacatcaggactggggtaaggaccattaccacgggcgcccccattacgtactccacctatggcaagttccttgccgacggtggttgctccgggggcgcttacgacatcataatgtgcgatgagtgccactcaactgactcaactactatcttgggcatcggcacagtcctggaccaagcggagacggctggagcgcggcttgtcgtgctcgccaccgctacgcctccaggatcggtcaccgtgccacaccccaatatcgaggaggtggccctgtcgaacactggagagatccccttctacggcaaagccatccccatcgaagccatcaaggggggaaggcacctcattttctgtcactccaagaagaagtgcgacgagcttgccgcaaagctgtcaggcctcggaatcaatgctgtagcgtattaccggggtcttgatgtgtccgtcataccgaccagcggagacgtcgttgtcgtggcaacagacgctctaatgacgggctataccggtgactttgattcagtgatcgactgtaatacgtgtgtcacccagacagtcgacttcagcttggaccccaccttcaccattgagacgacgaccgtgccccaagacgcagtgtcgcgctcgcagcggcggggtaggactggcaggggcagggggggcatatacaggtttgtaactccgggggaacggccctcgggcatgttcgattcctcggtcctgtgcgagtgctatgacgcgggctgtgcttggtacgagctcacccccgctgagacctcggttaggttgcgggcttacctaaatacaccaggattgcccgtttgccaggaccatctggagttctgggagagcgtcttcacaggcctcacccatatagatgcccacttcctgtcccagaccaagcaggcaggagataacttcccctacctggtggcataccaagccacagtgtgcgccagggctcaggccccacctccatcgtgggatcaaatgtggaagtgtctcatacggctaaaacccacgctgcacgggccaacgcccctgctgtataggctaggggccgtccaaaatgaggtcaccctcacacaccccataaccaaatacatcatggcatgcatgtcggccgacctggaagtcgtcaccagcacctgggtgctggtaggcggagtcctcgcagctctggccgcatattgcctgacaacaggcagtgtggttatcgtgggtaggatcatcttgtccgggaggccggctgtcgttcccgatagggaagtcctctaccgggagttcgatgaaatggaagaatgcgcctcgcacctcccttacatcgaacagggaatgcaactcgccgagcaattcaagcagaaggcgctcgggttgttgcaaacagccaccaagcaggcggaggctgccgctcccgtggtggagtccaagtggcgagctttggagaccttctgggcaaagcacatgtggaatttcatcagcgggatacagtacttagcgggcttatccaccctgcctgggaaccccgcgatagcatcactgatggcattcacagcctctatcaccagcccgctcaccacccagaacaccctcctgtttaacatcttgggggggtgggtagccgcccaactcgctccccccagcgctgcttcggctttcgtgggcgctggtatcgctggtgcggctgttggcagcataggtcttgggaaggtgctagtggacattctggcgggctatggggcaggggtggctggcgcgctcgtggccttcaaggtcatgagcggcgaggcgccctctgccgaggacctgatcaatttgctccctgccatcctctctcctggtgccctggtcgtcggagtcgtgtgtgcagcaatactgcgtcggcatgtgggcccgggagagggggccgtgcagtggatgaaccggctgatagcgttcgcttcgcggggtaaccatgtctcccccacgcactatgtgcctgagagcgacgccgcagcgcgtgtcactcaggtcctctccagccttaccatcacccagctgctgaagaggctccaccagtggattaatgaggactgttctacgccgtgttccggctcgtggctgagggatgtttgggactgggtgtgcacggtgttgagtgacttcaagacctggctccagtccaagctcctgccgcggttaccgggtgtccctttcctctcatgccaacgtgggtacaagggagtctggcggggggacggcatcatgcacaccacctgcccatgtggagcacagatcgccggacatgtcaaaaacggttccatgaggatcatcgggccgaaaacctgcagcaacacgtggcatggaacattccccatcaacgcgtacaccacgggcccctgcacgccttccccggcgccaaactattccaaggcgctgtggcgggtggctgctgaggagtacgtggaggtcacgcgggtgggggatttccactacgtgacgggcataaccaccgacaacgtaaagtgcccatgtcaggttccagctcctgagtttttcacggaggtggatggggtgcggttgcacaggtacgccccggtgtgcaaacctctcttacgggatgaggttgtattccaggtcgggctcaatcaatacctggttgggtcacagctcccatgcgagcccgaaccggacgtagcagtgctcacttccatgctcaccgacccctcccacattacagcagaggcggctaagcgtaggttggccagggggtctcccccctccttggccagctcttcagctagccagctgtctgcgccctccttgagggcgacatgcactacccattcttcctataatcttgactctccggacgtcgacctcattgaggccaacctcctgtggcggcaggagatgggcggaaacatcacccgcgtggagtcggagaacaaggtggtagtcctagactctttcgagccgcttcgagcggagggggatgagaatgaaatatccattgcggcggagatcctgcggaagtccaagaagttccccgcggcgatacccatatgggcacggccggattacaatcctccattgttagagtcttggaagaacccggactacgtccctccggtggtacacgggtgcccattgccacctgtcaaggcccctccaataccacctccacggagaaaaaggacggttgtcctgacggactccaccgtgtcttctgttttggcggagctcgctaccaaaaccttcggcagctccgaattgtcggccgccgacagcggcacggcgaccgcccctcctgaccagacctccgacaacggcggcaaagactccgacgctgagtcatgctcctctatgcccccccttgagggggagccgggggaccccgatctcagcgacgggtcttggtctaccgtgagcgaggaggctggtgagagcgtcgtctgctgctcaatgtcctacacatggacaggtgccctgatcacgccatgcgccgcggaagaaagcaagctgcccatcaacgcgttgagcaactctttgctgcgccatcacaacatggtctacgccacgacatcccgcagcgcgggcctgcggcagaagaaggtcacctttgacagactgcaggtcctggatgaccattaccgggacgtgcttaaggagatgaaggcaaaggcgtccacagtcaaggctaaacttctatccatagaagaagcctgccgcctgacgcccccacattcggccaaatccaagtttggctatggggcaaaggacgtccggaacctatccagcagggccatcaaccacatccgctccgtgtgggaggacttgctggaggacactgtgacaccaattgacaccaccgtcatggcaaagaatgaggttttctgcgtccaaccagagaagggaggccgcaagccagcccgccttatcgtattcccagatttgggagttcgtgtatgcgagaagatggctctctacgatgtggtctccacccttcctcaagccgtgatgggctcctcatacggattccagtactctcccgggcagcgggtcgagttcctggtaaaagcctggaaatcaaagaaaaaccctatgggcttctcatatgacacccgctgttttgactcaacggtcactgagaatgacatccgtgttgaggagtcaatttaccaatgttgtgacttggcccccgaagccagacaggctataaaatcgctcacagagcggctttatatcgggggtcccctgactaattcaaaagggcagagctgtggttatcgccggtgccgcgcgagcggcgtgctgacgactagctgcggtaataccctcacatgttacttgaaagcctctgccgcctgtcgagctgcaaagctccaggactgcacgatgctcgtgaacggggacgaccttgtcgttatctgcgaaagcgcgggaacccaggaggatgcggcgagcctacgagtcttcacggaggctatgactaggtactccgccccccccggggacttgccccaaccagaatacgacttggagttgataacatcatgttcctccaatgtgtcggtcgcgcacgatgcatctggcaaaagggtgtactacctcactcgcgatcccaccacccccatcgcacgggctgcgtgggaaacagctagacacactccagttaactcctggctaggcaacattatcatgtatgcgcccaccttatgggcaaggatgattctgatgacccatttcttctccatccttctagctcaggagcaacttgaaaaagccctggattgccaaatctacggggcctgttactccattgagccacttgacctacctcagatcattgaacgactccatggtcttagcgcattttcactccatagttactctccaggtgagatcaatagggtggcttcatgcctcaggaaacttggggtaccgcccttgcgagtctggagacatcgggccaggagcgtccgcgctaaactactgtcccagggggggagggccgccacttgcggcaaatacctcttcaactgggcagtaaagaccaagctcaaactcactccaatcccggctgcgtcccagttggacttatccggctggttcgttgctggctacagcgggggagacatatatcacagcctgtctcgtgcccgaccccgctggttcatgctgtgcctactcctactttctgtaggggtaggcatctacttgctccccaatcgatgaacggggagctaaacactccaggccaataggccatttcctgtttttttttttttttggtttttttttttttttttttttttttttttttttttttttcctttccttctttttttttttttccctctttatggtggctccatcttagccctagtcacggctagctgtgaaaggtccgtgagccgcatgactgcagagagtgctgatactggcctctctgcagatcatgt

SEQ ID NO: 15 - DNA, 8024 bp, Hepatitis C virus
acctgcccctaataggggcgacactccgccatgaatcactcccctgtgaggaactactgtcttcacgcagaaagcgcctagccatggcgttagtatgagtgtcgtacagcctccaggcccccccctcccgggagagccatagtggtctgcggaaccggtgagtacaccggaattgccgggaagactgggtcctttcttggataaacccactctatgcccggccatttgggcgtgcccccgcaagactgctagccgagtagcgttgggttgcgaaaggccttgtggtactgcctgatagggcgcttgcgagtgccccgggaggtctcgtagaccgtgcaccatgagcacaaatcctaaacctcaaagaaaaaccaaaagaaacaccaaccgtcgcccaatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttaaaccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataccatggctcccatcactgcttatgcccagcaaacacgaggcctcctgggcgccatagtggtgagtatgacggggcgtgacaggacagaacaggccggggaagtccaaatcctgtccacagtctctcagtccttcctcggaacaaccatctcgggggttttgtggactgtttaccacggagctggcaacaagactctagccggcttacggggtccggtcacgcagatgtactcgagtgctgagggggacttggtaggctggcccagcccccctgggaccaagtctttggagccgtgcaagtgtggagccgtcgacctatatctggtcacgcggaacgctgatgtcatcccggctcggagacgcggggacaagcggggagcattgctctccccgagacccatttcgaccttgaaggggtcctcgggggggccggtgctctgccctaggggccacgtcgttgggctcttccgagcagctgtgtgctctcggggcgtggccaaatccatcgatttcatccccgttgagacactcgacgttgttacaaggtctcccactttcagtgacaacagcacgccaccggctgtgccccagacctatcaggtcgggtacttgcatgctccaactggcagtggaaagagcaccaaggtccctgtcgcgtatgccgcccaggggtacaaagtactagtgcttaacccctcggtagctgccaccctggggtttggggcgtacctatccaaggcacatggcatcaatcccaacattaggactggagtcaggaccgtgatgaccggggaggccatcacgtactccacatatggcaaatttctcgccgatgggggctgcgctagcggcgcctatgacatcatcatatgcgatgaatgccacgctgtggatgctacctccattctcggcatcggaacggtccttgatcaagcagagacagccggggtcagactaactgtgctggctacggccacaccccccgggtcagtgacaaccccccatcccgatatagaagaggtaggcctcgggcgggagggtgagatccccttctatgggagggcgattcccctatcctgcatcaagggagggagacacctgattttctgccactcaaagaaaaagtgtgacgagctcgcggcggcccttcggggcatgggcttgaatgccgtggcatactatagagggttggacgtctccataataccagctcagggagatgtggtggtcgtcgccaccgacgccctcatgacggggtacactggagactttgactccgtgatcgactgcaatgtagcggtcacccaagctgtcgacttcagcctggaccccaccttcactataaccacacagactgtcccacaagacgctgtctcacgcagtcagcgccgcgggcgcacaggtagaggaagacagggcacttataggtatgtttccactggtgaacgagcctcaggaatgtttgacagtgtagtgctttgtgagtgctacgacgcaggggctgcgtggtacgatctcacaccagcggagaccaccgtcaggcttagagcgtatttcaacacgcccggcctacccgtgtgtcaagaccatcttgaattttgggaggcagttttcaccggcctcacacacatagacgcccacttcctctcccaaacaaagcaagcgggggagaacttcgcgtacctagtagcctaccaagctacggtgtgcgccagagccaaggcccctcccccgtcctgggacgccatgtggaagtgcctggcccgactcaagcctacgcttgcgggccccacacctctcctgtaccgtttgggccctattaccaatgaggtcaccctcacacaccctgggacgaagtacatcgccacatgcatgcaagctgaccttgaggtcatgaccagcacgtgggtcctagctggaggagtcctggcagccgtcgccgcatattgcctggcgactggatgcgtttccatcatcggccgcttgcacgtcaaccagcgagtcgtcgttgcgccggataaggaggtcctgtatgaggcttttgatgagatggaggaatgcgcctctagggcggctctcatcgaagaggggcagcggatagccgagatgttgaagtccaagatccaaggcttgctgcagcaggcctctaagcaggcccaggacatacaacccgctatgcaggcttcatggcccaaagtggaacaattttgggccagacacatgtggaacttcattagcggcatccaatacctcgcaggattgtcaacactgccagggaaccccgcggtggcttccatgatggcattcagtgccgccctcaccagtccgttgtcgaccagtaccaccatccttctcaacatcatgggaggctggttagcgtcccagatcgcaccacccgcgggggccaccggctttgtcgtcagtggcctggtgggggctgccgtgggcagcataggcctgggtaaggtgctggtggacatcctggcaggatatggtgcgggcatttcgggggccctcgtcgcattcaagatcatgtctggcgagaagccctctatggaagatgtcatcaatctactgcctgggatcctgtctccgggagccctggtggtgggggtcatctgcgcggccattctgcgccgccacgtgggaccgggggagggcgcggtccaatggatgaacaggcttattgcctttgcttccagaggaaaccacgtcgcccctactcactacgtgacggagtcggatgcgtcgcagcgtgtgacccaactacttggctctcttactataaccagcctactcagaagactccacaattggataactgaggactgccccatcccatgctccggatcctggctccgcgacgtgtgggactgggtttgcaccatcttgacagacttcaaaaattggctgacctctaaattgttccccaagctgcccggcctccccttcatctcttgtcaaaaggggtacaagggtgtgtgggccggcactggcatcatgaccacgcgctgcccttgcggcgccaacatctctggcaatgtccgcctgggctctatgaggatcacagggcctaaaacctgcatgaacacctggcaggggacctttcctatcaattgctacacggagggccagtgcgcgccgaaaccccccacgaactacaagaccgccatctggagggtggcggcctcggagtacgcggaggtgacgcagcatgggtcgtactcctatgtaacaggactgaccactgacaatctgaaaattccttgccaactaccttctccagagtttttctcctgggtggacggtgtgcagatccataggtttgcacccacaccaaagccgtttttccgggatgaggtctcgttctgcgttgggcttaattcctatgctgtcgggtcccagcttccctgtgaacctgagcccgacgcagacgtattgaggtccatgctaacagatccgccccacatcacggcggagactgcggcgcggcgcttggcacggggatcacctccatctgaggcgagctcctcagtgagccagctatcagcaccgtcgctgcgggccacctgcaccacccacagcaacacctatgacgtggacatggtcgatgccaacctgctcatggagggcggtgtggctcagacagagcctgagtccagggtgcccgttctggactttctcgagccaatggccgaggaagagagcgaccttgagccctcaataccatcggagtgcatgctccccaggagcgggtttccacgggccttaccggcttgggcacggcctgactacaacccgccgctcgtggaatcgtggaggaggccagattaccaaccgcccaccgttgctggttgtgctctccccccccccaagaaggccccgacgcctcccccaaggagacgccggacagtgggtctgagcgagagcaccatatcagaagccctccagcaactggccatcaagacctttggccagcccccctcgagcggtgatgcaggctcgtccacgggggcgggcgccgccgaatccggcggtccgacgtcccctggtgagccggccccctcagagacaggttccgcctcctctatgccccccctcgagggggagcctggagatccggacctggagtctgatcaggtagagcttcaacctcccccccagggggggggggtagctcccggttcgggctcggggtcttggtctacttgctccgaggaggacgataccaccgtgtgctgctccatgtcatactcctggaccggggctctaataactccctgtagccccgaagaggaaaagttgccaatcaaccctttgagtaactcgctgttgcgataccataacaaggtgtactgtacaacatcaaagagcgcctcacagagggctaaaaaggtaacttttgacaggacgcaagtgctcgacgcccattatgactcagtcttaaaggacatcaagctagcggcttccaaggtcagcgcaaggctcctcaccttggaggaggcgtgccagttgactccaccccattctgcaagatccaagtatggattcggggccaaggaggtccgcagcttgtccgggagggccgttaaccacatcaagtccgtgtggaaggacctcctggaagacccacaaacaccaattcccacaaccatcatggccaaaaatgaggtgttctgcgtggaccccgccaaggggggtaagaaaccagctcgcctcatcgtttaccctgacctcggcgtccgggtctgcgagaaaatggccctctatgacattacacaaaagcttcctcaggcggtaatgggagcttcctatggcttccagtactcccctgcccaacgggtggagtatctcttgaaagcatgggcggaaaagaaggaccccatgggtttttcgtatgatacccgatgcttcgactcaaccgtcactgagagagacatcaggaccgaggagtccatataccaggcctgctccctgcccgaggaggcccgcactgccatacactcgctgactgagagactttacgtaggagggcccatgttcaacagcaagggtcaaacctgcggttacagacgttgccgcgccagcggggtgctaaccactagcatgggtaacaccatcacatgctatgtgaaagccctagcggcctgcaaggctgcggggatagttgcgcccacaatgctggtatgcggcgatgacctagtagtcatctcagaaagccaggggactgaggaggacgagcggaacctgagagccttcacggaggccatgaccaggtactctgcccctcctggtgatccccccagaccggaatatgacctggagctaataacatcctgttcctcaaatgtgtctgtggcgttgggcccgcggggccgccgcagatactacctgaccagagacccaaccactccactcgcccgggctgcctgggaaacagttagacactcccctatcaattcatggctgggaaacatcatccagtatgctccaaccatatgggttcgcatggtcctaatgacacacttcttctccattctcatggtccaagacaccctggaccagaacctcaactttgagatgtatggatcagtatactccgtgaatcctttggaccttccagccataattgagaggttacacgggcttgacgccttttctatgcacacatactctcaccacgaactgacgcgggtggcttcagccctcagaaaacttggggcgccacccctcagggtgtggaagagtcgggctcgcgcagtcagggcgtccctcatctcccgtggagggaaagcggccgtttgcggccgatatctcttcaattgggcggtgaagaccaagctcaaactcactccattgccggaggcgcgcctactggacttatccagttggttcaccgtcggcgccggcgggggcgacatttttcacagcgtgtcgcgcgcccgaccccgctcattactcttcggcctactcctacttttcgtaggggtaggcctcttcctactccccgctcggtagagcggcacacactaggtacactccatagctaactgttcctttttttttttttttttttttttttttttttttttttttttttcttttttttttttttccctctttcttcccttctcatcttattctactttctttcttggtggctccatcttagccctagtcacggctagctgtgaaaggtccgtgagccgcatgactgcagagagtgccgtaactggtctctctgcagatcatgt

SEQ ID NO: 16 - DNA, 12567 bp, Hepatitis C virus
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccgtttaaacccccgtgctgctggaagtcgatttcaggcttagggtaaccgtggacctcgaaaacagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattggccatccggtgaaagtgagactcattatctatctgtttgctggatccgctccattgagtgtgtttactctaagtacaatttcaacagttatttcaatcagacaattgtatcataatggcgggcccagaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaagcggccgcgttgttaaacagaccacaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatgggaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaatcggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggacggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcgtaggctggccaggggatctcccccctccttggccagctcatcagctagccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacggggagctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagtactactagtccctttagtgagggttaattcaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagctaagctgcctgcaggtaatacgactcactata

SEQ ID NO: 17 - DNA, 10907 bp, Hepatitis C virus
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaacgggcgcgccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagttctcgccgatagtggaaaccgacgccccagcactcgtccggatcgggagatgggggaggctaacggtttaaacatgcagatcttcgtgaagaccctgacgggcaagaccatcactcttgaggtcgagcccagtgacaccatcgagaatgtcaaggccaagatccaagacaaggaaggcatcccacctgaccagcagaggctgatattcgcgggcaaacagctggaggatggccgcaccctgtccgactacaacatccagaaagagtccaccttgcacctggtgctgcgtctccgcggtggtgcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatgggaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaatcggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggtgacttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccatcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggaaggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggggcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacgacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggatgtagcagtgctcacttccatgctcaccgacccctcccacattacggcggagacggctaagcgtaggctggccaggggatctcctccccccttggccagctcatcagctagccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccaccttcacggaggaagaggacggttgtcctgtcagaatctaccgtgtcttctgccttggcggagctcgccacagagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacggggagctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagtactactagtccctttagtgagggttaattcaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagctaagcttcgtaatacgactcactata

SEQ ID NO: 18 - DNA, 11691 bp, Hepatitis C virus
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaacgggcgcgccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttaaacagaccacaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatggaccgggagatggcagcatcgtgcggaggcgcggttttcgtaggtctgatactcttgaccttgtcaccgcactataagctgttcctcgctaggctcatatggtggttacaatattttatcaccagggccgaggcacacttgcaagtgtggatcccccccctcaacgttcgggggggccgcgatgccgtcatcctcctcacgtgcgcgatccacccagagctaatctttaccatcaccaaaatcttgctcgccatactcggtccactcatggtgctccaggctggtataaccaaagtgccgtacttcgtgcgcgcacacgggctcattcgtgcatgcatgctggtgcggaaggttgctgggggtcattatgtccaaatggctctcatgaagttggccgcactgacaggtacgtacgtttatgaccatctcaccccactgcgggactgggcccacgcgggcctacgagaccttgcggtggcagttgagcccgtcgtcttctctgatatggagaccaaggttatcacctggggggcagacaccgcggcgtgtggggacatcatcttgggcctgcccgtctccgcccgcagggggagggagatacatctgggaccggcagacagccttgaagggcaggggtggcgactcctcgcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatggaaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaaccggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggaaggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcgtaggctggccaggggatctcccccctccttggccagctcatcagctagccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacggggagctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagtactactagtccctttagtgagggttaattcaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagctaagcttcgtaatacgactcactata

SEQ ID NO: 19 - DNA, 11901 bp, Hepatitis C virus
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaacgggcgcgccatggaagacgccaaaaacataaaggaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaagtttaaacagaccacaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatggaaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaaccggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggaaggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcgtaggctggccaggggatctcccccctccttggccagctcatcagctagccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaaggactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacggggagctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagtactactagtccctttagtgagggttaattcaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagctaagcttcgtaatacgactcactata

SEQ ID NO: 20 - DNA, 11901 bp, Hepatitis C virus
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaacgggcgcgccatggaagacgccaaaaacataaaggaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaagtttaaacagaccacaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatgggaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaatcggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggtgacttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccatcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggaaggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggggcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacgacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggatgtagcagtgctcacttccatgctcaccgacccctcccacattacggcggagacggctaagcgtaggctggccaggggatctcctccccccttggccagctcatcagctagccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccaccttcacggaggaagaggacggttgtcctgtcagaatctaccgtgtcttctgccttggcggagctcgccacagagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacggggagctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagtactactagtccctttagtgagggttaattcaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagctaagcttcgtaatacgactcactata

SEQ ID NO: 21 - DNA, 11901 bp, Hepatitis C virus
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaacgggcgcgccatggaagacgccaaaaacataaaggaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaagtttaaacagaccacaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatgggaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaatcggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggacggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcgtaggctggccaggggatctcccccctccttggccagctcatcagctagccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacggggagctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagtactactagtccctttagtgagggttaattcaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagctaagcttcgtaatacgactcactata

SEQ ID NO: 22 - DNA, 12939 bp, Hepatitis C virus
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaacgggcgcgccatggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgggtttaaacatgcagatcttcgtgaagaccctgacgggcaagaccatcactcttgaggtcgagcccagtgacaccatcgagaatgtcaaggccaagatccaagacaaggaaggcatcccacctgaccagcagaggctgatattcgcgggcaaacagctggaggatggccgcaccctgtccgactacaacatccagaaagagtccaccttgcacctggtgctgcgtctccgcggtggtatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcggccgcgttgttaaacagaccacaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatgggaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaatcggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggacggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcgtaggctggccaggggatctcccccctccttggccagctcatcagctagccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacggggagctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagtactactagtccctttagtgagggttaattcaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagctaagcttggtaatacgactcactat

SEQ ID NO: 23 - DNA, 4244 bp, Hepatitis C virus
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaacgggcgcgccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttctagtccctttagtgagggttaattcaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagctaagcttcgtaatacgactcactata

SEQ ID NO: 24 - DNA, 14117 bp, Hepatitis C virus
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaacgggcgcgccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttaaacagaccacaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatgggcacgaatcctaaacctcaaagaaaaaccaaacgtaacaccaaccgccgcccacaggacgtcaagttcccgggcggtggtcagatcgtcggtggagtttacctgttgccgcgcaggggccccaggttgggtgtgcgcgcgactaggaagacttccgagcggtcgcaacctcgtggaaggcgacaacctatccccaaggctcgccagcccgagggtagggcctgggctcagcccgggtacccctggcccctctatggcaatgagggcttggggtgggcaggatggctcctgtcaccccgtggctctcggcctagttggggccccacggacccccggcgtaggtcgcgcaatttgggtaaggtcatcgataccctcacgtgcggcttcgccgatctcatggggtacattccgctcgtcggcgcccccctagggggcgctgccagggccctggcgcatggcgtccgggttctggaggacggcgtgaactatgcaacagggaatctgcccggttgctccttttctatcttccttttggctttgctgtcctgtttgaccatcccagcttccgcttatgaagtgcgcaacgtatccggagtgtaccatgtcacgaacgactgctccaacgcaagcattgtgtatgaggcagcggacatgatcatgcatacccccgggtgcgtgccctgcgttcgggagaacaactcctcccgctgctgggtagcgctcactcccacgctcgcggccaggaacgctagcgtccccactacgacgatacgacgccatgtcgatttgctcgttggggcggctgctctctgctccgctatgtacgtgggagatctctgcggatctgttttcctcgtcgcccagctgttcaccttctcgcctcgccggcacgagacagtacaggactgcaattgctcaatatatcccggccacgtgacaggtcaccgtatggcttgggatatgatgatgaactggtcacctacagcagccctagtggtatcgcagttactccggatcccacaagctgtcgtggatatggtggcgggggcccattggggagtcctagcgggccttgcctactattccatggtggggaactgggctaaggttctgattgtgatgctactctttgccggcgttgacgggggaacctatgtgacaggggggacgatggccaaaaacaccctcgggattacgtccctcttttcacccgggtcatcccagaaaatccagcttgtaaacaccaacggcagctggcacatcaacaggactgccctgaactgcaatgactccctcaacactgggttccttgctgcgctgttctacgtgcacaagttcaactcatctggatgcccagagcgcatggccagctgcagccccatcgacgcgttcgctcaggggtgggggcccatcacttacaatgagtcacacagctcggaccagaggccttattgttggcactacgcaccccggccgtgcggtatcgtacccgcggcgcaggtgtgtggtccagtgtactgcttcaccccaagccctgtcgtggtggggacgaccgaccggttcggcgtccctacgtacagttggggggagaatgagacggacgtgctgcttcttaacaacacgcggccgccgcaaggcaactggtttggctgtacatggatgaatagcactgggttcaccaagacgtgcgggggccccccgtgtaacatcggggggatcggcaataaaaccttgacctgccccacggactgcttccggaagcaccccgaggccacttacaccaagtgtggttcggggccttggttgacacccagatgcttggtccactacccatacaggctttggcactacccctgcactgtcaactttaccatcttcaaggttaggatgtacgtggggggagtggagcacaggctcgaagccgcatgcaattggactcgaggagagcgttgtaacctggaggacagggacagatcagagcttagcccgctgctgctgtctacaacggagtggcaggtattgccctgttccttcaccaccctaccggctctgtccactggtttgatccatctccatcagaacgtcgtggacgtacaatacctgtacggtatagggtcggcggttgtctcctttgcaatcaaatgggagtatgtcctgttgctcttccttcttctggcggacgcgcgcgtctgtgcctgcttgtggatgatgctgctgatagctcaagctgaggccgccctagagaacctggtggtcctcaacgcggcatccgtggccggggcgcatggcattctctccttcctcgtgttcttctgtgctgcctggtacatcaagggcaggctggtccctggggcggcatatgccctctacggcgtatggccgctactcctgctcctgctggcgttaccaccacgagcatacgccatggaccgggagatggcagcatcgtgcggaggcgcggttttcgtaggtctgatactcttgaccttgtcaccgcactataagctgttcctcgctaggctcatatggtggttacaatattttatcaccagggccgaggcacacttgcaagtgtggatcccccccctcaacgttcgggggggccgcgatgccgtcatcctcctcacgtgcgcgatccacccagagctaatctttaccatcaccaaaatcttgctcgccatactcggtccactcatggtgctccaggctggtataaccaaagtgccgtacttcgtgcgcgcacacgggctcattcgtgcatgcatgctggtgcggaaggttgctgggggtcattatgtccaaatggctctcatgaagttggccgcactgacaggtacgtacgtttatgaccatctcaccccactgcgggactgggcccacgcgggcctacgagaccttgcggtggcagttgagcccgtcgtcttctctgatatggagaccaaggttatcacctggggggcagacaccgcggcgtgtggggacatcatcttgggcctgcccgtctccgcccgcagggggagggagatacatctgggaccggcagacagccttgaagggcaggggtggcgactcctcgcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatgggaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaatcggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggtgacttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccatcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggaaggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggggcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacgacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggatgtagcagtgctcacttccatgctcaccgacccctcccacattacggcggagacggctaagcgtaggctggccaggggatctcctccccccttggccagctcatcagctagccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccaccttcacggaggaagaggacggttgtcctgtcagaatctaccgtgtcttctgccttggcggagctcgccacagagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacggggagctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagtactactagtccctttagtgagggttaattcaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagctaagcttggtaatacgactcactat

SEQ ID NO: 25 - DNA, 11042 bp, Hepatitis C virus
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaacgggcgcgccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttaaacagaccacaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatgggaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaatcggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggtgacttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccatcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggaaggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggggcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacgacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggatgtagcagtgctcacttccatgctcaccgacccctcccacattacggcggagacggctaagcgtaggctggccaggggatctcctccccccttggccagctcatcagctagccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccaccttcacggaggaagaggacggttgtcctgtcagaatctaccgtgtcttctgccttggcggagctcgccacagagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacggggagctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagtactactagtccctttagtgagggttaattcaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagctaagcttggtaatacgactcactat

SEQ ID NO: 26 - DNA, 11043 bp, Hepatitis C virus
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaacgggcgcgccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttaaacagaccacaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatggaaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaaccggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggaaggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcgtaggctggccaggggatctcccccctccttggccagctcatcagctagccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaaggactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacggggagctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagtactactagtccctttagtgagggttaattcaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagctaagcttcgtaatacgactcactata

SEQ ID NO: 27 - DNA, 11043 bp, Hepatitis C virus
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaacgggcgcgccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttaaacagaccacaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatggaaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaaccggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggaaggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcgtaggctggccaggggatctcccccctccttggccagctcatcagctagccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacggggagctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagtactactagtccctttagtgagggttaattcaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagctaagcttcgtaatacgactcactata

SEQ ID NO: 28 - DNA, 28 bp, Artificial Sequence
  Description of Artificial Sequence Synthetic Primer
acttgatctgcagagaggccagtatcag

SEQ ID NO: 29 - DNA, 33 bp, Artificial Sequence
  Description of Artificial Sequence Synthetic Primer
cccggctagcatggcgcctattacggcctactc

SEQ ID NO: 30 - DNA, 37 bp, Artificial Sequence
  Description of Artificial Sequence Synthetic Primer
ccggaattcttagcactcttccatctcatcgaactcc

(19) United States
(12) Patent Application Publication
(10) Pub. No.: US 2008/0207760 A1
(43) Pub. Date: Aug. 28, 2008

(54) Pharmaceutical Compositions For and Methods of Inhibiting Hcv

(75) Inventor: Mingjun Huang, Potomac, MD (US)

Correspondence Address: CANTOR COLBURN, LLP, 20 Church Street, 22nd Floor, Hartford, CT, 06103, US

(73) Assignee: Achillion Pharmaceuticals, Inc., New Haven, CT (US)

(21) Appl. No.: 11/911,330

(22) PCT Filed: Apr. 11, 2006
(86) PCT No.: PCT/US06/13503

§ 371 Date: Oct. 11, 2007

Related U.S. Application Data
(60) Provisional application No. 60/669,872, filed on Apr. 11, 2005.

(51) Int. Cl. A61K 31/17; C12Q 1/70; C07C 335/00; A61P 31/14

(52) U.S. Cl. 514/580; 435/5; 564/17

(57) ABSTRACT

The present invention relates generally to replicase complex defect inducers and pharmaceutical compositions containing such inducers. Methods of developing mutants that are resistant to replicase complex defect inducers are also provided. Further included are mutants that can be used in screening for replicase complex defect inducers. Methods of screening test compounds for the ability to induce the formation of replicase complex defects are also described. Also included are methods of inhibition of HCV replication by replicase complex defect inducers.

### REFERENCE TO RELATED APPLICATION

The present application claims the benefit of U.S. Provisional Application No. 60/669,872, filed Apr. 11, 2005, which application is herein incorporated by reference in its entirety.

### INCORPORATION OF SEQUENCE LISTING

A paper copy of the Sequence Listing submitted herewith is herein incorporated by reference.

### FIELD OF THE INVENTION

The present invention relates generally to replicase complex defect inducers and pharmaceutical compositions containing such inducers. Methods of developing mutants that are resistant to replicase complex defect inducers are also provided. Further included are mutants that can be used in screening for replicase complex defect inducers. Methods of screening test compounds for the ability to induce the formation of replicase complex defects are also described. Also included are methods of inhibition of HCV replication by replicase complex defect inducers.

### BACKGROUND

Hepatitis C Virus (HCV) is one of the most prevalent causes of chronic liver disease in the United States, accounting for about 15 percent of acute viral hepatitis, 60 to 70 percent of chronic hepatitis, and up to 50 percent of cirrhosis, end-stage liver disease, and liver cancer. Almost 4 million Americans, or about 1.8 percent of the U.S. population, have antibodies to HCV (i.e., anti-HCV antibodies), indicating previous or ongoing infection with the virus. Hepatitis C causes an estimated 8,000 to 10,000 deaths annually in the United States. While the acute phase of HCV infection is usually associated with mild symptoms, some evidence suggests that only about 15% to 20% of infected people will clear HCV.

HCV is a small, enveloped, single-stranded, positive strand RNA virus in the Flaviviridae family. The HCV lifecycle includes entry into host cells; translation of the HCV genome, polyprotein processing, and replicase complex assembly; RNA replication, and virion assembly and release. Translation of the HCV RNA genome yields a more than 3000 amino acid long polyprotein that is processed by at least two cellular and two viral proteases. The HCV polyprotein is:

NH₂—C-E1-E2-p7-NS2-NS3-NS4A-NS4B-NS5A-NS5B-COOH.

The cellular signal peptidase and signal peptide peptidase have been reported to be responsible for cleavage of the N-terminal third of the polyprotein (C-E1-E2-p7) from the nonstructural proteins (NS2-NS3-NS4A-NS4B-NS5A-NS5B). The NS2-NS3 protease mediates a first cis cleavage at the NS2-NS3 site. The NS3-NS4A protease then mediates a second cis-cleavage at the NS3-NS4A junction. The NS3-NS4A complex then cleaves at 3 downstream sites to separate the remaining nonstructural proteins. Accurate processing of the polyprotein is asserted to be essential for forming an active HCV replicase complex.

Once the polyprotein has been cleaved, the replicase complex comprising at least the NS3-NS5B nonstructural proteins assembles. The replicase complex is cytoplasmic and membrane-associated. Major enzymatic activities in the replicase complex include serine protease activity and NTPase helicase activity in NS3, and RNA-dependent RNA polymerase activity of NS5B. In the RNA replication process, a complementary negative strand copy of the genomic RNA is produced. The negative strand copy is used as a template to synthesize additional positive strand genomic RNAs that may participate in translation, replication, packaging, or any combination thereof to produce progeny virus.

Previously studied targets for drug discovery include the NS3-NS4A protease and the NS5B polymerase. The protease domain of the NS3-NS4A protease includes the N-terminal third of NS3 and a short stretch of NS4A, which has been reported to function as a cofactor. A high-resolution structure of the protease has enabled the development of protease inhibitors which are either substrate analogs, inhibitors containing a serine trap, or product-mimicking inhibitors. NS3 also includes a helicase domain in the C-terminal 500 amino acids, the structure of which has enabled the development of small molecule inhibitors of helicase function. The NS5B polymerase has also been a target for high resolution structural studies and drug design. Inhibitors of viral polymerases include substrate (nucleoside) analogs, product (pyrophosphate) analogs, and nonnucleoside inhibitors.

While these previously known HCV inhibitors are suitable for their intended purpose, there nonetheless remains a need for additional HCV inhibitors, particularly those that operate by distinct mechanisms.

### SUMMARY OF THE INVENTION

The present invention includes and provides a method of identifying a mutant that is resistant to a replicase complex defect inducer comprising: growing HCV virus in cells; adding a selection agent and a test compound to the cells; and identifying a mutant that is resistant to the test compound and sensitive to an NS5B polymerase inhibitor and an NS3 protease inhibitor.

The present invention also includes and provides a method of identifying a mutant that is resistant to a replicase complex defect inducer comprising: growing cells that express an HCV replicon; adding a selection agent and a test compound to the cells; and identifying a mutant that is resistant to the test compound and sensitive to an NS5B polymerase inhibitor and an NS3 protease inhibitor.

The present invention further includes and provides a method of identifying a mutant that is resistant to a replicase complex defect inducer comprising: growing cells that express an isolated HCV replicase complex; adding a selection agent and a test compound to the cells; and identifying a mutant that is resistant to the test compound and sensitive to an NS5B polymerase inhibitor and an NS3 protease inhibitor.

The present invention includes and provides a method of identifying a mutant that is resistant to a replicase complex defect inducer comprising: growing cells that express an isolated HCV polyprotein or fragment thereof; adding a selection agent and a test compound to the cells; and identifying a mutant that is resistant to the test compound and sensitive to an NS5B polymerase inhibitor and an NS3 protease inhibitor.

The present invention also includes and provides a method of identifying a mutation that results in viral growth in the presence of an HCV replicase complex defect inducer comprising: generating a population of mutants comprising an HCV virion with a mutation in a nonstructural protein of HCV; identifying a mutant that is resistant to a test compound and sensitive to an NS5B polymerase inhibitor and an NS3 protease inhibitor; and determining the nucleotide sequence of the mutation.

The present invention also includes and provides a method of identifying a mutation that results in viral growth in the presence of an HCV replicase complex defect inducer comprising: generating a population of mutants comprising an HCV replicon with a mutation in a nonstructural protein of HCV; identifying a mutant that is resistant to a test compound and sensitive to an NS5B polymerase inhibitor and an NS3 protease inhibitor; and determining the nucleotide sequence of the mutation.

The present invention includes and provides a method of identifying a mutation that results in viral growth in the presence of an HCV replicase complex defect inducer comprising: generating a population of mutants comprising an isolated HCV replicase complex with a mutation in a nonstructural protein of HCV; identifying a mutant that is resistant to a test compound and sensitive to a NS5B polymerase inhibitor and a NS3 protease inhibitor; and determining the nucleotide sequence of the mutation.

The present invention also includes and provides a method of identifying a mutation that results in viral growth in the presence of an HCV replicase complex defect inducer comprising: generating a population of mutants comprising an isolated HCV polyprotein or fragment thereof with a mutation in a nonstructural protein of HCV; identifying a mutant that is resistant to a test compound and sensitive to a NS5B polymerase inhibitor and a NS3 protease inhibitor; and determining the nucleotide sequence of the mutation.

The present invention also includes and provides a method of determining resistance to a test compound comprising: introducing into a cell an HCV virion comprising a mutation; contacting a test compound with the cell; and measuring the resistance of the virion to the test compound.

The present invention includes and provides a method of determining resistance to a test compound comprising: introducing into a cell an HCV replicon comprising a mutation; contacting a test compound with the cell; and measuring the resistance of the cell to the test compound.

The present invention further includes and provides a method of determining resistance to a test compound comprising: introducing into a cell an HCV replicon comprising a mutation; contacting a test compound with the cell; and measuring the resistance of the replicon to the test compound.

The present invention also includes and provides a method of determining resistance to a test compound comprising: introducing into a cell an isolated HCV replicase complex comprising a mutation; contacting a test compound with the cell; and measuring the resistance of the replicase complex to the test compound.

The present invention also includes and provides a method of determining resistance to a test compound comprising: introducing into a cell an isolated HCV polyprotein or fragment thereof comprising a mutation; contacting a test compound with the cell; and measuring the resistance of the HCV polyprotein or fragment thereof to the test compound.

The present invention further includes and provides a method of screening a test compound for replicase complex defect inducer activity comprising: providing a test compound; contacting the test compound with a cell infected by an HCV virion that comprises an NS3 protein with a mutation at or within about 15 angstroms of C16; and identifying the test compound as an inducer of an HCV replicase complex defect when the virion is resistant to the test compound.

The present invention includes and provides a method of screening a test compound for replicase complex defect inducer activity comprising: providing a test compound; contacting the test compound with a cell infected by an HCV replicon that comprises an NS3 protein with a mutation at or within about 15 angstroms of C16; and identifying the test compound as an inducer of an HCV replicase complex defect when the HCV replicon is resistant to the test compound.

The present invention includes and provides a method of screening a test compound for replicase complex defect inducer activity comprising: providing a test compound; contacting the test compound with a cell expressing an isolated HCV replicase complex that comprises an NS3 protein with a mutation at or within about 15 angstroms of C16; and identifying the test compound as an inducer of an HCV replicase complex defect when the HCV replicase complex is resistant to the test compound.

The present invention includes and provides a method of screening a test compound for replicase complex defect inducer activity comprising: providing a test compound; contacting the test compound with a cell expressing an isolated HCV polyprotein that comprises an NS3 protein with a mutation that is at or within about 15 angstroms of C16; and identifying the test compound as an inducer of an HCV replicase complex defect when the HCV polyprotein is resistant to the test compound.

The present invention includes and provides a method of screening a test compound for replicase complex defect inducer activity comprising: providing a test compound; contacting the test compound with a cell that is infected with an HCV virion; and identifying the test compound as an inducer of an HCV replicase complex defect.

The present invention further includes and provides a method of screening a test compound for replicase complex defect inducer activity comprising: providing a test compound; contacting the test compound with a cell that is infected with an HCV replicon; and identifying the test compound as an inducer of an HCV replicase complex defect.

The present invention also includes and provides a method of screening a test compound for replicase complex defect inducer activity comprising: providing a test compound; contacting the test compound with a cell that expresses an isolated HCV replicase complex, and identifying the test compound as an inducer of an HCV replicase complex defect.

The present invention includes and provides a method of screening a test compound for replicase complex defect inducer activity comprising: providing a test compound; contacting the test compound with a cell that expresses an isolated HCV polyprotein or fragment thereof; and identifying the test compound as an inducer of an HCV replicase complex defect.

The present invention also includes and provides a method of screening a test compound for replicase complex defect inducer activity comprising: providing a test compound; contacting the test compound with an HCV virion; and identifying the test compound as an inducer of an HCV replicase complex defect when the level of p14 protein is increased.

The present invention further includes and provides a method of screening a test compound for replicase complex defect inducer activity comprising: providing a test compound; contacting the test compound with an HCV replicon; and identifying the test compound as an inducer of an HCV replicase complex defect when the level of p14 protein is increased.

The present invention also includes and provides a method of screening a test compound for replicase complex defect inducer activity comprising: providing a test compound; contacting the test compound with an isolated HCV replicase complex; and identifying the test compound as an inducer of an HCV replicase complex defect when the level of p14 protein is increased.

The present invention also includes and provides a method of screening a test compound for replicase complex defect inducer activity comprising: providing a test compound; contacting the test compound with an isolated HCV polyprotein or fragment thereof; and identifying the test compound as an inducer of an HCV replicase complex defect when the level of p14 protein is increased.

The present invention further includes and provides a method of screening a test compound for replicase complex defect inducer activity comprising: providing a test compound; contacting the test compound with a cell that expresses an isolated HCV polyprotein or fragment thereof, wherein the HCV polyprotein or fragment thereof comprises an NS4A protein; and identifying the test compound as an inducer of an HCV replicase complex defect.

The present invention also includes and provides a method of screening a test compound for replicase complex defect inducer activity comprising: providing a test compound; contacting the test compound with an isolated HCV polyprotein or fragment thereof, wherein the HCV polyprotein or fragment thereof comprises an NS4A protein; and identifying the test compound as an inducer of an HCV replicase complex defect when the level of p14 protein is increased.

### BRIEF DESCRIPTION OF THE DRAWINGS

FIG. 1 is an exemplary schematic of a mechanism of action of the RCDIs.

FIG. 2 depicts mutations in Cys16 or Ala39.

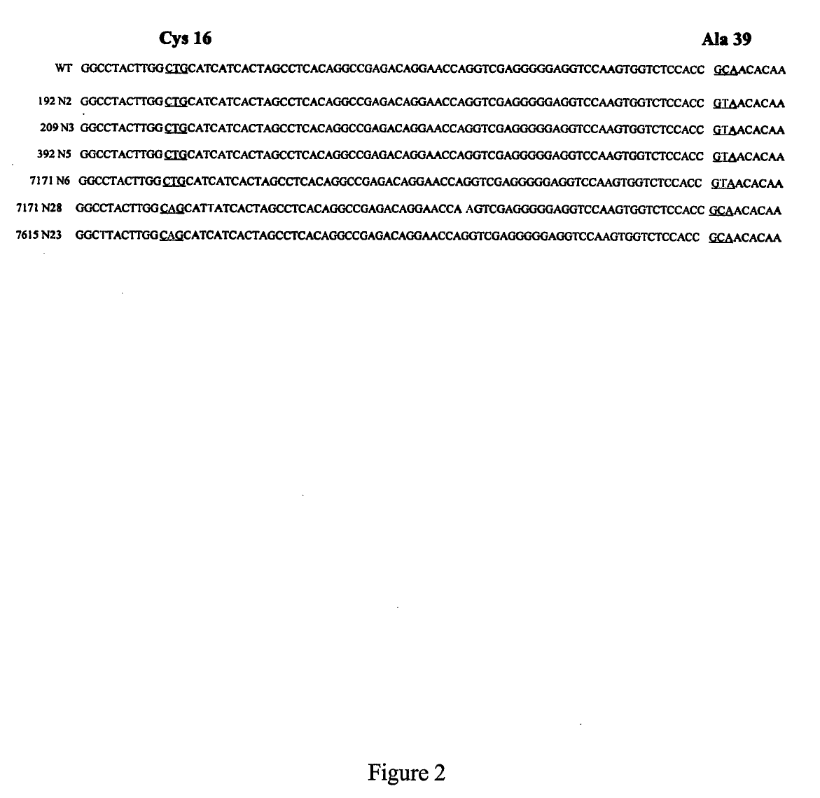

FIG. 3 shows that [³H] labeled azidoacylthiourea, 1-(5-Azido-benzofuran-2-carbonyl)-3-(4-pentyloxy-3-trifluoromethyl-phenyl)-thiourea, binds to synthetic NS4A.

FIG. 4 shows the chemical structures of acylthioureas, 1-(5-Azido-benzofuran-2-carbonyl)-3-(4-pentyloxy-3-trifluoromethyl-phenyl)-thiourea, 1-(5-Azido-benzofuran-2-carbonyl)-3-(4-pentyloxy-3-trifluoromethyl-phenyl)-urea. 1-(Benzofuran-2-carbonyl)-3-(4-pentyloxy-3-trifluoromethyl-phenyl)-thiourea and 1-(4-Pentyloxy-3-trifluoromethyl-phenyl)-3-(pyridine-3-carbonyl)-thiourea.

FIG. 5 shows that acylthioureas 1-(Benzofuran-2-carbonyl)-3-(4-pentyloxy-3-trifluoromethyl-phenyl)-thiourea and 1-(4-Pentyloxy-3-trifluoromethyl-phenyl)-3-(pyridine-3-carbonyl)-thiourea effectively compete with [³H] labeled 1-(5-Azido-benzofuran-2-carbonyl)-3-(4-pentyloxy-3-trifluoromethyl-phenyl)-thiourea for NS4A binding.

FIG. 6 shows that 1-(5-Azido-benzofuran-2-carbonyl)-3-(4-pentyloxy-3-trifluoromethyl-phenyl)-urea does not compete with [³H] labeled 1-(5-Azido-benzofuran-2-carbonyl)-3-(4-pentyloxy-3-trifluoromethyl-phenyl)-thiourea for NS4A binding.

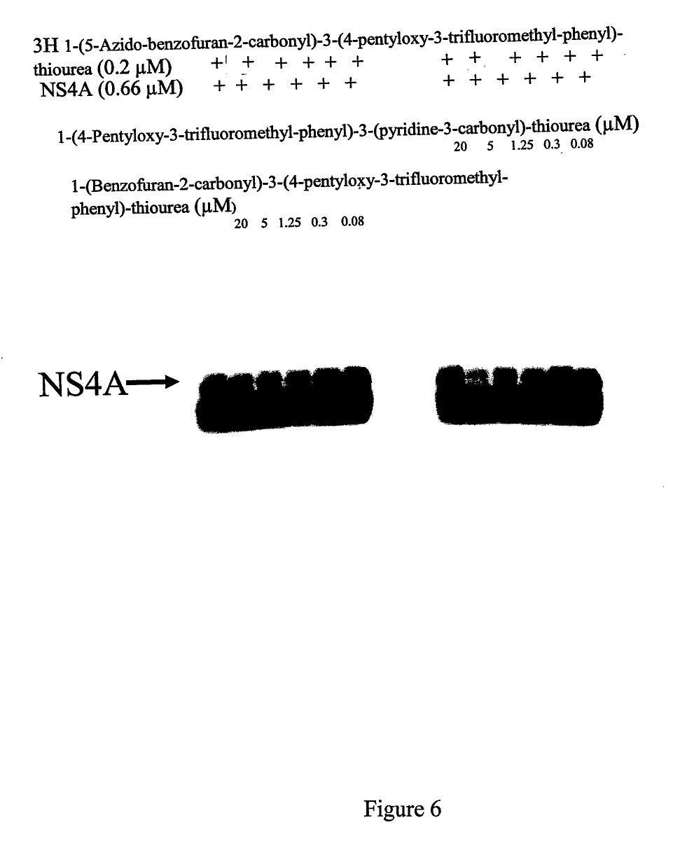

### DETAILED DESCRIPTION OF THE INVENTION

### Non-Limiting Embodiments

1. A method of identifying a mutant that is resistant to a replicase complex defect inducer comprising:

growing cells that express an HCV replicon;

adding G418 and a test compound; and

identifying a mutant that is resistant to a test compound and sensitive to a NS5B polymerase inhibitor and a NS3B protease inhibitor.

2. The method according to claim 1, wherein the cells are Huh-7 cells.
3. The method according to claim 1, wherein the HCV replicon is a Con-1 replicon.
4. The method according to claim 1, wherein G418 is added at a concentration from about 500 μg/mL to about 1 mg/mL.
5. The method according to claim 1, where the mutant has a mutation in an NS3 protein or fragment thereof.
6. The method according to claim 1, wherein the mutant has a mutation at or within about 15 angstroms of C16 in an NS3 protein or fragment thereof.
7. The method according to claim 1, wherein the mutant has an A39V mutation in an NS3 protein or fragment thereof.
8. The method according to claim 1, wherein the mutant has a C16S mutation in an NS3 protein or fragment thereof.
9. A mutant identified by the method of claim 1.
10. A method of identifying a mutant that is resistant to a replicase complex defect inducer comprising:

growing cells that express an isolated HCV replicase complex;

adding G418 and a test compound; and

identifying a mutant that is resistant to a test compound and sensitive to a NS5B polymerase inhibitor and a NS3B protease inhibitor.

11. The method according to claim 10, wherein the cells are Huh-7 cells.
12. The method according to claim 10, wherein G418 is added at a concentration from about 500 μg/mL to about 1 mg/mL.
13. The method according to claim 10, where the mutant has a mutation in an NS3 protein or fragment thereof.
14. The method according to claim 10, wherein the mutant has a mutation at or within about 15 angstroms of C16 in an NS3 protein or fragment thereof.
15. The method according to claim 10, wherein the mutant has an A39V mutation in an NS3 protein or fragment thereof.
16. The method according to claim 10, wherein the mutant has a C16S mutation in an NS3 protein or fragment thereof.
17. A mutant identified by the method of claim 10.
18. A method of identifying a mutant that is resistant to a replicase complex defect inducer comprising:

growing cells that express an isolated HCV polyprotein or fragment thereof;

adding G418 and a test compound; and

identifying a mutant that is resistant to a test compound and sensitive to a NS5B polymerase inhibitor and a NS3B protease inhibitor.

19. The method according to claim 18, wherein the cells are Huh-7 cells.
20. The method according to claim 18, wherein G418 is added at a concentration from about 500 μg/mL to about 1 mg/mL.
21. The method according to claim 18, where the mutant has a mutation in an NS3 protein or fragment thereof.
22. The method according to claim 18, wherein the mutation is at or within about 15 angstroms of C16 in an NS3 protein or fragment thereof
23. The method according to claim 18, wherein the mutant has an A39V mutation in an NS3 protein or fragment thereof.
24. The method according to claim 18, wherein the mutant has a C16S mutation in an NS3 protein or fragment thereof
25. The method according to claim 18, wherein the isolated HCV polyprotein comprises NS3 protein or fragment thereof.
26. The method according to claim 18, wherein the isolated HCV polyprotein comprises NS3-NS4A or a fragment thereof.
27. A mutant identified by the method of claim 18.
28. A method of identifying a mutation that causes resistance to growth in the presence of an HCV replicase complex defect inducer comprising:

generating a population of mutants comprising an HCV replicon with a mutation in a nonstructural protein of HCV;

identifying a mutant that is resistant to a test compound and sensitive to a NS5B polymerase inhibitor and a NS3B protease inhibitor; and

determining the nucleotide sequence of the mutation.

29. The method according to claim 28, wherein the mutation is in an NS3 protein or fragment thereof.
30. The method according to claim 28, wherein the mutation is at or within about 15 angstroms of C16 in an NS3 protein or fragment thereof.
31. The method according to claim 28, wherein the mutation is an A39V mutation in an NS3 protein or fragment thereof.
32. The method according to claim 28, wherein the mutation is a C16S mutation in an NS3 protein or fragment thereof.
33. The method according to claim 28, wherein the mutation in a nonstructural protein of HCV has not previously been identified.
34. A mutation identified by the method of claim 28.
35. A method of identifying a mutation that causes resistance to growth in the presence of an HCV replicase complex defect inducer comprising:

generating a population of mutants comprising an isolated HCV replicase complex with a mutation in a nonstructural protein of HCV;

identifying a mutant that is resistant to a test compound and sensitive to a NS5B polymerase inhibitor and a NS3B protease inhibitor; and

determining the nucleotide sequence of the mutation.

36. The method according to claim 35, wherein the mutation is in an NS3 protein or fragment thereof.
37. The method according to claim 35, wherein the mutation is at or within about 15 angstroms of C16 in an NS3 protein or fragment thereof.
38. The method according to claim 35, wherein the mutation is an A39V mutation in an NS3 protein or fragment thereof.
39. The method according to claim 35, wherein the mutation is a C16S mutation in an NS3 protein or fragment thereof.
40. The method according to claim 35, wherein the mutation in a nonstructural protein of HCV has not previously been identified.
41. A mutation identified by the method of claim 35.
42. A method of identifying a mutation that causes resistance to growth in the presence of an HCV replicase complex defect inducer comprising:

generating a population of mutants comprising an isolated HCV polyprotein or fragment thereof with a mutation in a nonstructural protein of HCV;

identifying a mutant that is resistant to a test compound and sensitive to a NS5B polymerase inhibitor and a NS3B protease inhibitor; and

determining the nucleotide sequence of the mutation.

43. The method according to claim 42, wherein the mutation is in an NS3 protein or fragment thereof.
44. The method according to claim 42, wherein the mutation is at or within about 15 angstroms of C16 in an NS3 protein or fragment thereof.
45. The method according to claim 42, wherein the mutation is an A39V mutation in an NS3 protein or fragment thereof.
46. The method according to claim 42, wherein the mutation is a C16S mutation in an NS3 protein or fragment thereof.
47. The method according to claim 42, wherein the mutation in a nonstructural protein of HCV has not previously been identified.
48. A mutation identified by the method of claim 42.
49. A method of determining resistance to a test compound comprising:

introducing into a cell an HCV replicon comprising a mutation; and

contacting a test compound with the cell; and

measuring the resistance of the cell to the test compound.

50. The method according to claim 49, wherein the cell is a Huh-7 cell.
51. The method according to claim 49, wherein the HCV replicon is a Con-1 replicon.
52. The method according to claim 49, wherein the mutation is in an NS3 protein or fragment thereof.
53. The method according to claim 49, wherein the mutation is at or within about 15 angstroms of C16 in an NS3 protein or fragment thereof.
54. The method according to claim 49, wherein the mutation is an A39V mutation in an NS3 protein or fragment thereof
55. The method according to claim 49, wherein the mutation is a C16S mutation in an NS3 protein or fragment thereof
56. The method according to claim 49, wherein measuring the resistance of the cell to the test compound comprises determining the EC₅₀ or the EC₉₀, of the test compound.
57. The method according to claim 56, wherein determining the EC₅₀ or the EC₉₀ is done by performing an RNA dot blot protocol.
58. A method of determining resistance to a test compound comprising:

introducing into a cell an isolated HCV replicase complex comprising a mutation; and

contacting a test compound with the cell; and

measuring the resistance of the cell to the test compound.

59. The method according to claim 58, wherein the cell is a Huh-7 cell.
60. The method according to claim 58, wherein the mutation in an NS3 protein or fragment thereof.
61. The method according to claim 58, wherein the mutation is at or within about 15 angstroms of C16 in an NS3 protein or fragment thereof.
62. The method according to claim 58, wherein the mutation is an A39V mutation in an NS3 protein or fragment thereof.
63. The method according to claim 58, wherein the mutation is a C16S mutation in an NS3 protein or fragment thereof.
64. The method according to claim 58, wherein measuring the resistance of the cell to the test compound comprises determining the EC₅₀ or the EC₉₀, of the test compound.
65. The method according to claim 64, wherein determining the EC₅₀ or the EC₉₀ is done by performing an RNA dot blot protocol.
66. A method of determining resistance to a test compound comprising:

introducing into a cell an isolated HCV polyprotein or fragment thereof comprising a mutation; and

contacting a test compound with the cell; and

measuring the resistance of the cell to the test compound.

67. The method according to claim 66, wherein the cell is a Huh-7 cell.
68. The method according to claim 66, wherein the HCV polyprotein or fragment thereof comprises NS3 protein or a fragment thereof.
69. The method according to claim 66, wherein the HCV polyprotein or fragment thereof comprises NS3-NS4A or a fragment thereof.
70. The method according to claim 66, wherein the mutation is in an NS3 protein or fragment thereof.
71. The method according to claim 66, wherein the mutation is at or within about 15 angstroms of C16 in an NS3 protein.
72. The method according to claim 66, wherein the mutation is an A39V mutation in an NS3 protein.
73. The method according to claim 66, wherein the mutation is a C16S mutation in an NS3 protein.
74. The method according to claim 66, wherein measuring the resistance of the cell to the test compound comprises determining the EC₅₀ or the EC₉₀, of the test compound.
75. The method according to claim 74, wherein determining the EC₅₀ or the EC₉₀ is done by performing an RNA dot blot protocol.
76. A method of screening a test compound for replicase complex defect inducer activity comprising:
providing a test compound;
contacting the test compound with a cell expressing an HCV replicon that comprises an NS3 protein with a mutation at or within about 15 angstroms of C16; and
identifying the test compound as an inducer of an HCV replicase complex defect when the cell is resistant to the test compound.
   77. The method according to claim 76, wherein the cell is an Huh-7 cell.
   78. The method according to claim 76, wherein the HCV replicon is a Con-1 replicon.
   79. The method according to claim 76, wherein the mutation that is at or within about 15 angstroms of C16 is an A39V mutation.
   80. The method according to claim 76, wherein the mutation that is at or within about 15 angstroms of C16 is a C16S mutation.
   81. The method according to claim 76, wherein determining resistance to the test compound comprises determining the EC₅₀ or the EC₉₀ of the test compound.
   82. The method according to claim 81, wherein determining the EC₅₀ or the EC₉₀ is done by performing an RNA dot blot protocol.
   83. The method according to claim 76, wherein the test compound has not previously been screened for induction of an HCV replicase complex defect.
   84. The method according to claim 76, wherein the test compound has not previously been identified as an inducer of an HCV replicase complex defect.
   85. An inducer of a replicase complex defect identified by the method of claim 76, wherein said inducer is a compound of Formula (III)

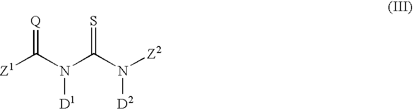

wherein
Q is oxygen or sulfur;
D¹ and D² are independently selected from the group consisting of hydrogen and methyl;
with the proviso that said compound of Formula (III) is not a compound of the formula (III-a):

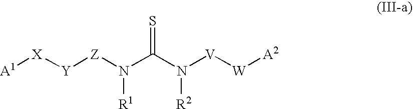

wherein

A₁ and A₂ are independently optionally substituted C₁-C₁₂ alkyl, optionally substituted mono- or di-(C₁-C₈ alkyl)amino, optionally substituted C₂-C₁₂ alkenyl, optionally substituted C₃-C₈ cycloalkyl, an optionally substituted partially unsaturated or aromatic carbocyclic group, or an optionally substituted saturated, partially unsaturated, or aromatic heterocyclic group, wherein at least one of A₁ and A₂ is an optionally substituted aromatic carbocyclic group or an optionally substituted aromatic heterocyclic group;

X and W are independently O, S, NR, or absent, wherein R is hydrogen, optionally substituted (C₁-C₆)alkyl, or optionally substituted aryl(C₀-C₄)alkyl;

V is (C₁-C₆)alkyl, (C₂-C₆)alkenyl, (C₃-C₇)cycloalkyl, or absent, wherein when V is absent, W is absent; and

Y is (C₁-C₆) alkyl, (C₁-C₆)alkyl substituted with (C₃-C₇)cycloalkyl, (C₂-C₆) alkenyl, (C₃-C₇)cycloalkyl, or absent; and

Z is carbonyl, thiocarbonyl, imino, or C₁-C₆ alkylimino; and

R₁ and R₂ are independently hydrogen or methyl;

and with the further proviso that said compound of Formula (III) is not a compound of the formula (III-b):

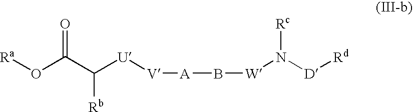

wherein
R^{a} is hydrogen, a substituted or unsubstituted alkyl or cycloalkyl group, a substituted or unsubstituted aryl group or a saturated or unsaturated, optionally substituted heterocyclic group;
R^{b} is hydrogen, a substituted or unsubstituted alkyl or cycloalkyl group, a substituted or unsubstituted aryl group, a saturated or unsaturated, optionally substituted heterocyclic group, an optionally substituted alkenyl group, an optionally substituted alkinyl group, —NR^{2′}SO₂R^{2″}, —NR^{2′}COOR^{2′}, —NR^{2′}COR^{2′}, —NR^{2′}CON(R^{2′})₂, or —N R^{2′}CSN(R^{2′})₂;
R^{2′} is hydrogen, a substituted or unsubstituted alkyl or cycloalkyl group, a substituted or unsubstituted aryl group or a saturated or unsaturated, optionally substituted heterocyclic group;
R^{2″} is a substituted or unsubstituted alkyl, alkenyl or cycloalkyl group, a substituted or unsubstituted aryl group, or a saturated or unsaturated, optionally substituted heterocyclic group;
U′ is a direct bond or a substituted or unsubstituted alkylene group;
V′ is a substituted or unsubstituted alkylene group, —NR^{2′}CO—, or —NR^{2′}SO₂—;
A and B are each independently an optionally substituted 1,3- or 1,4-bridging phenylene group or an optionally substituted 2,4- or 2,5-bridging thienylene group,
W′ is a direct bond or an optionally substituted alkylene group;
D′ is a direct bond or

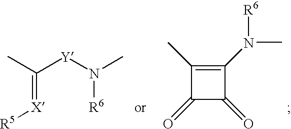

R^{c} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{d}, Y′, R⁵, or R⁶, if present, with formation of an optionally substituted heterocyclic ring system which includes the nitrogen atom to which R^{c} is bonded, and may be saturated or unsaturated and may contain further heteroatoms;
R^{d} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{c}, Y, R⁵ or R⁶, if present, with formation of an optionally substituted heterocyclic ring system which includes the nitrogen atom to which R^{d} is bonded and may be saturated or unsaturated and may contain further heteroatoms;

### X′ is CHNO₂, CHCN, O, N or S;

Y′ is a direct bond or an optionally substituted alkylene or alkine group;
R⁵ is absent, or is hydrogen, a substituted or unsubstituted alkyl or cycloalkyl group, —NO₂, —CN, —COR^{5′}, —COOR^{5′}, or is connected to one of R^{c}, Y′, R^{d}, or R⁶, if present, with formation of an optionally substituted carbocyclic or heterocyclic ring system which includes X′ and can be saturated or unsaturated and may optionally contain one or more additional heteroatoms;
R^{5′} is hydrogen, a substituted or unsubstituted alkyl or cycloalkyl group, a substituted or unsubstituted aryl group or a saturated or unsaturated, optionally substituted heterocyclic group which may optionally contain one or more additional heteroatoms;
R⁶ is a arylcarbonyl group, or a heteroarylcarbonyl group;
and wherein if A is a phenylene group and V′ is —NR^{2′}CO— or —NR^{2′} SO₂—, D′ is not a direct bond and X′ is not N;
and with the further proviso that said compound of Formula (III) is not a compound of the formula (III-c)

X^{1″}—Y^{1″}-Z^{1″}-W^{1″} (III-c)

wherein
X^{1″} is optionally substituted aryl, optionally substituted (C₁-C₈)alkyl-aryl, optionally substituted heteroaryl, or optionally substituted heteroarylalkyl;
Y^{1″} is —NR^{A}CSNR^{A}CO—, wherein R^{A} is independently hydrogen or lower alkyl;
Z^{1″} is optionally substituted phenylene, optionally substituted monocyclic heteroarylene, optionally substituted monocyclic non-aromatic heterocycle-diyl, or optionally substituted monocyclic cycloalkane-diyl;
W^{1″} is a group represented by the formula:

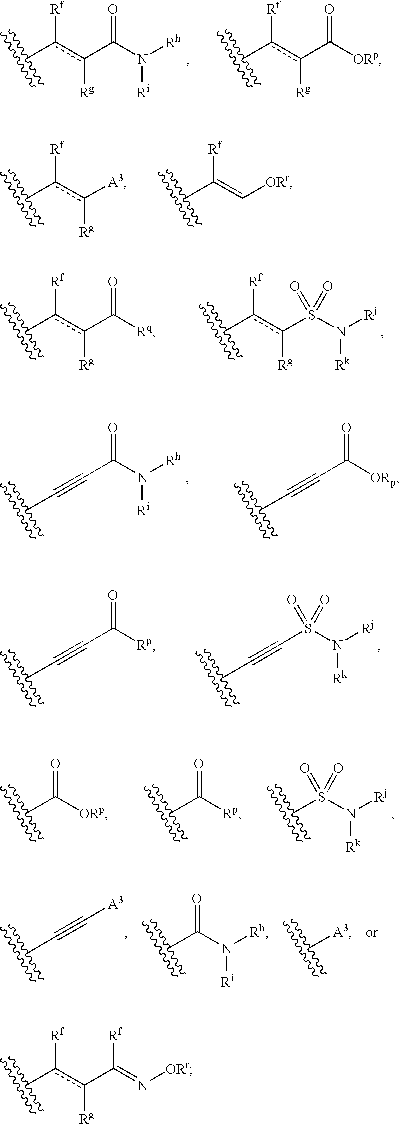

wherein
R^{f}, R^{g}, R^{h}, Rⁱ, R^{j}, and R^{k} are each independently hydrogen, halogen, optionally substituted lower alkyl, optionally substituted lower alkyloxy, optionally substituted lower alkylthio, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, optionally substituted non-aromatic heterocyclic group, or optionally substituted amino;
R^{p}, R^{q}, and R^{r} are each independently hydrogen, optionally substituted lower alkyl, optionally substituted lower alkenyl, optionally substituted lower alkynyl, optionally substituted aryl, optionally substituted heteroaryl, optionally substituted cycloalkyl, optionally substituted (C₁-C₉)alkyl-aryl, optionally substituted heteroarylalkyl, or optionally substituted non-aromatic heterocyclic group;
A³ is an optionally substituted aryl or an optionally substituted heteroaryl group;
and with the further proviso that in said compound of Formula (III), Z¹ is not a 3-pyridyl group or a substituted 3-pyridyl group when Z² is —OH, —NH₂, —(C₁-C₆)alkylamino, di(C₁-C₆)alkylamino, an alkyl group, a substituted alkyl group, —(C₃-C₈) cycloalkyl group, a substituted —(C₃-C₈) cycloalkyl group, —(C₃-C₈) cycloalkyl-(C₁-C₆)alkyl group, —O-alkyl group, a substituted —O-alkyl group, —O—(C₃-C₈) cycloalkyl group, a substituted —O—(C₃-C₈) cycloalkyl group, a phenyl group, a fused-phenyl group, a phenyl group substituted with one or more independently selected halogen, —OCH₃, nitro, or dimethyl amino groups, a substituted fused-phenyl group substituted with one or more independently selected halogen, —OCH₃, nitro, or dimethyl amino groups, a —(C₁-C₈) alkyl-phenyl group, —O—(C₀-C₈) alkyl-phenyl group, —(C₃-C₆) alkylene group, —O—(C₃-C₆) alkylene group, —(C₃-C₆) alkynyl group, —O—(C₃-C₆) alkynyl group, —CO(C₁-C₆) alkyl group, —NHCO(C₁-C₆) alkyl group, —NHSO₂(C₁-C₆) alkyl group, —SO₂(C₁-C₆) alkyl group, or —SO₂(C₀-C₆)alkyl-phenyl group;
and with the further proviso that in said compound of Formula (III), Z¹ is not a furyl group or a substituted furyl group when D² and Z² are independently selected from the group consisting of hydrogen and methyl;
and with the further proviso that in said compound comprising structure (III), when D² is —CH₃ and Z² is an optionally substituted phenyl, Z¹ is not selected from the group consisting of —R³³, OR³⁴, and —NR³⁵R³⁶,
wherein
R³³ is hydrogen, optionally substituted (C₃-C₈)cycloalkyl, optionally substituted phenyl, optionally substituted heteroaryl or phenyl-(C₁-C₄) alkyl which is optionally substituted on the phenyl ring, (C₁-C₆)alkyl, (C₂-C₆)alkenyl, (C₂-C₆)alkynyl, the three latter radicals independently being optionally substituted with one or more radicals selected from the group consisting of halogen, (C₁-C₄)alkoxy, (C₁-C₄)alkylthio and NR³⁷R³⁸;
R³⁴ is (C₁-C₆)alkyl, (C₂-C₆)alkenyl, (C₂-C₆)alkynyl, the three latter radicals independently optionally substituted with one or more radicals selected from the group consisting of halogen, (C₁-C₄)alkoxy, (C₁-C₄)alkylthio and NR³⁷R³⁸, or R³⁴ is (C₃-C₆)cycloalkyl optionally substituted with one or more groups selected from the group consisting of halogen, (C₁-C₄)alkyl and (C₁-C₄)alkoxy, and (C₃-C₆)-cycloalkyl-(C₁-C₃)alkyl;
R³⁵ and R³⁶ are hydrogen, (C₁-C₆)alkyl, (C₂-C₆)alkenyl, (C₂-C₆)alkynyl, the 3 latter groups independently optionally substituted by one or more halogen radicals;
R³⁷ is independently selected from the group consisting of hydrogen, (C₁-C₄)alkyl, ((C₁-C₄)alkyl)carbonyl, ((C₁-C₄)alkoxy)carbonyl and CHO;
R³⁸ is independently selected from the group consisting of H and (C₁-C₄)alkyl;
and with the further proviso that in said compound of Formula (III), Z¹ and Z² are not both selected from the group consisting of hydrogen and alkyl;
and with the further proviso that in said compound of Formula (III), Z² is not hydrogen when D¹ and D² are both hydrogen;
and with the further proviso that in said compound of Formula (III), Z¹ is not (C₁-C₆) alkyl, (C₁-C₆) alkoxy, pyridyl, or aryl when D¹ and D² are both hydrogen and when Z² is selected from the group consisting of hydrogen, a (C₁-C₆) alkyl group optionally substituted with 1 to 3 substituents independently selected from the group consisting of hydroxyl, (C₁-C₆) alkoxyl, carboxyl, (C₁-C₆) alkoxycarbonyl, lower alkylthio, fluorine, chlorine, bromine, iodine, amino, mono- or di-substituted amino, phthalimido and nitrogen-containing heterocyclic groups, a (C₃-C₈) cycloalkyl group, a (C₂-C₆) alkenyl group, an arylalkyl group optionally substituted with from 1 to 5 substituents independently selected from the group consisting of (C₁-C₆) alkyl, hydroxyl, (C₁-C₆) alkoxy, halogen, nitro, and amino groups, an aryl group optionally substituted with from 1 to 5 independently selected substituents selected from the group consisting of (C₁-C₆) alkyl, hydroxyl, (C₁-C₆) alkoxy, halogen, nitro, and amino groups, a substituted or unsubstituted heterocyclic group, a (C₁-C₆) alkanoyl group, a benzoyl or a naphthoyl group either of which is optionally substituted with from 1 to 5 independently selected substituents selected from the group consisting of (C₁-C₆) alkyl, hydroxyl, (C₁-C₆) alkoxy, halogen, nitro, and amino groups, a pyridylcarbonyl group, a (C₁-C₆) alkoxycarbonyl group or an amino group optionally substituted with (C₁-C₆) alkyl, arylalkyl having 7 to 15 carbon atoms, substituted or unsubstituted aryl, (C₁-C₆) alkanoyl, optionally substituted aroyl, and (C₁-C₆) alkylidene groups;
and with the further proviso that in said compound of Formula (III), Z¹ is not —O—(CH₂)ₜ—CH₃, wherein t is 0-4 and Z² is halophenyl;
and with the further proviso that in said compound of Formula (III), Z¹ is not a substituted pyridyl, pyrimidinyl, pyrazinyl, pyridazinyl group substituted with —N(R²¹)-(optionally substituted phenyl), wherein said R²¹ is hydrogen, aryl, formyl, (C₁-C₆)alkylcarbonyl, (C₁-C₆)alkyl, (C₁-C₆)alkyloxycarbonyl, (C₁-C₆)alkyl substituted with formyl, (C₁-C₆)alkylcarbonyl, (C₁-C₆)alkyloxycarbonyl, (C₁-C₆)alkylcarbonyloxy, or (C₁-C₆)alkyloxy(C₁-C₆)alkylcarbonyl optionally substituted with (C₁-C₆)alkyloxycarbonyl;
and with the further proviso that in said compound of Formula (III), Z¹ is not a 3-(4-trifluoromethyl-pyridinyl) group or a 3-(4-trifluoromethyl-pyridinyl N-oxide) group;
and with the further proviso that in said compound of Formula (II), Z¹ is not a substituted or unsubstituted group selected from the group consisting of

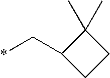

and a —(CH₂)ₜ-adamantanyl group, wherein t is 0-4;
and with the further proviso that in said compound of Formula (III), Z² is not a

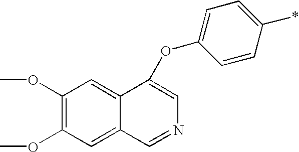

group, which is unsubstituted, or which is substituted with one or more substituents independently selected from the group consisting of halogens and alkyl groups;
and with the further proviso that in said compound of Formula (III), Z² is not a substituted or unsubstituted group selected from the group consisting of:

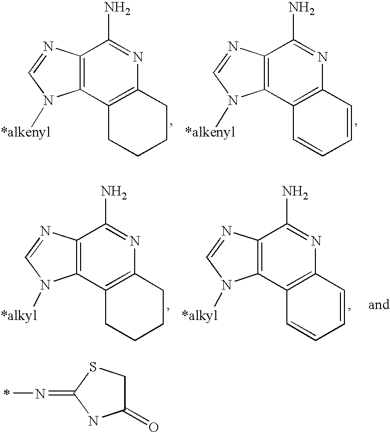

and with the further proviso that said compound of Formula (III), is not a compound set forth in Appendix A, Appendix B, or Appendix C.
86. A method of screening a test compound for replicase complex defect inducer activity comprising:
providing a test compound;
contacting the test compound with a cell expressing an isolated HCV replicase complex that comprises an NS3 protein with a mutation at or within about 15 angstroms of C16; and
identifying the test compound as an inducer of an HCV replicase complex defect when the cell is resistant to the test compound.
   87. The method according to claim 86, wherein the cell is an Huh-7 cell.
   88. The method according to claim 86, wherein the mutation that is at or within about 15 angstroms of C16 is an A39V mutation.
   89. The method according to claim 86, wherein the mutation that is at or within about 15 angstroms of C16 is a C16S mutation.
   90. The method according to claim 86, wherein determining resistance to the test compound comprises determining the EC₅₀ or the EC₉₀ of the test compound.
   91. The method according to claim 90, wherein determining the EC₅₀ or the EC₉₀ is done by performing an RNA dot blot protocol.
   92. The method according to claim 86, wherein the test compound has not previously been screened for induction of an HCV replicase complex defect.
   93. The method according to claim 86, wherein the test compound has not previously been identified as an inducer of an HCV replicase complex defect.
   94. An inducer of a replicase complex defect identified by the method of claim 86, wherein said inducer a compound of Formula (III)

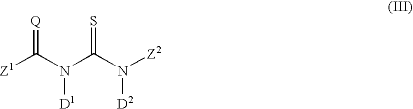

wherein
Q is oxygen or sulfur;
D¹ and D² are independently selected from the group consisting of hydrogen and methyl;
with the proviso that said compound of Formula (III) is not a compound of the formula (III-a):

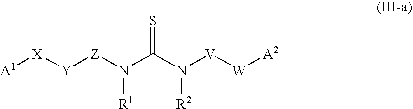

wherein

A₁ and A₂ are independently optionally substituted C₁-C₁₂ alkyl, optionally substituted mono- or di-(C₁-C₈ alkyl)amino, optionally substituted C₂-C₁₂ alkenyl, optionally substituted C₃-C₈ cycloalkyl, an optionally substituted partially unsaturated or aromatic carbocyclic group, or an optionally substituted saturated, partially unsaturated, or aromatic heterocyclic group, wherein at least one of A₁ and A₂ is an optionally substituted aromatic carbocyclic group or an optionally substituted aromatic heterocyclic group;

X and W are independently O, S, NR, or absent, wherein R is hydrogen, optionally substituted (C₁-C₆)alkyl, or optionally substituted aryl(C₀-C₄)alkyl;

V is (C₁-C₆)alkyl, (C₂-C₆)alkenyl, (C₃-C₇)cycloalkyl, or absent, wherein when V is absent, W is absent; and

Y is (C₁-C₆) alkyl, (C₁-C₆)alkyl substituted with (C₃-C₇)cycloalkyl, (C₂-C₆) alkenyl, (C₃-C₇)cycloalkyl, or absent; and

Z is carbonyl, thiocarbonyl, imino, or C₁-C₆ alkylimino; and

R₁ and R₂ are independently hydrogen or methyl;

and with the further proviso that said compound of Formula (III) is not a compound of the formula (III-b):

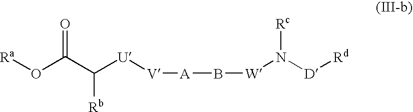

wherein
R^{a} is hydrogen, a substituted or unsubstituted alkyl or cycloalkyl group, a substituted or unsubstituted aryl group or a saturated or unsaturated, optionally substituted heterocyclic group;
R^{b} is hydrogen, a substituted or unsubstituted alkyl or cycloalkyl group, a substituted or unsubstituted aryl group, a saturated or unsaturated, optionally substituted heterocyclic group, an optionally substituted alkenyl group, an optionally substituted alkinyl group, —NR^{2′}SO₂R^{2″}, —NR^{2′}COOR^{2′}, —NR^{2′}COR^{2′}, —NR^{2′}CON(R^{2′})₂, or —N R^{2′}CSN(R^{2′})₂;
R^{2′} is hydrogen, a substituted or unsubstituted alkyl or cycloalkyl group, a substituted or unsubstituted aryl group or a saturated or unsaturated, optionally substituted heterocyclic group;
R^{2″} is a substituted or unsubstituted alkyl, alkenyl or cycloalkyl group, a substituted or unsubstituted aryl group, or a saturated or unsaturated, optionally substituted heterocyclic group;
U′ is a direct bond or a substituted or unsubstituted alkylene group;
V′ is a substituted or unsubstituted alkylene group, —NR^{2′}CO—, or —NR^{2′}SO₂—;
A and B are each independently an optionally substituted 1,3- or 1,4-bridging phenylene group or an optionally substituted 2,4- or 2,5-bridging thienylene group;
W′ is a direct bond or an optionally substituted alkylene group;
D′ is a direct bond or

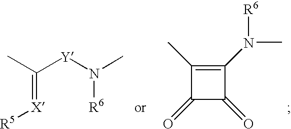

R^{c} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{d}, Y′, R⁵, or R⁶, if present, with formation of an optionally substituted heterocyclic ring system which includes the nitrogen atom to which R^{c} is bonded, and may be saturated or unsaturated and may contain further heteroatoms;
R^{d} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{c}, Y, R⁵ or R⁶, if present, with formation of an optionally substituted heterocyclic ring system which includes the nitrogen atom to which R^{d} is bonded and may be saturated or unsaturated and may contain further heteroatoms;

### X′ is CHNO₂, CHCN, O, N or S;

Y′ is a direct bond or an optionally substituted alkylene or alkine group;
R⁵ is absent, or is hydrogen, a substituted or unsubstituted alkyl or cycloalkyl group, —NO₂, —CN, —COR^{5′}, —COOR^{5′}, or is connected to one of R^{c}, Y′, R^{d}, or R⁶, if present, with formation of an optionally substituted carbocyclic or heterocyclic ring system which includes X′ and can be saturated or unsaturated and may optionally contain one or more additional heteroatoms;
R^{5′} is hydrogen, a substituted or unsubstituted alkyl or cycloalkyl group, a substituted or unsubstituted aryl group or a saturated or unsaturated, optionally substituted heterocyclic group which may optionally contain one or more additional heteroatoms;
R⁶ is a arylcarbonyl group, or a heteroarylcarbonyl group;
and wherein if A is a phenylene group and V′ is —NR^{2′}CO— or —NR^{2′}SO₂—, D′ is not a direct bond and X′ is not N;
and with the further proviso that said compound of Formula (III) is not a compound of the formula (III-c)

X^{1″}—Y^{1″}-Z^{1″}-W^{1″} (III-c)

wherein
X^{1″} is optionally substituted aryl, optionally substituted (C₁-C₈)alkyl-aryl, optionally substituted heteroaryl, or optionally substituted heteroarylalkyl;
Y^{1″} is —NR^{A}CSNR^{A}CO—, wherein R^{A} is independently hydrogen or lower alkyl;
Z^{1″} is optionally substituted phenylene, optionally substituted monocyclic heteroarylene, optionally substituted monocyclic non-aromatic heterocycle-diyl, or optionally substituted monocyclic cycloalkane-diyl;
W^{1″} is a group represented by the formula:

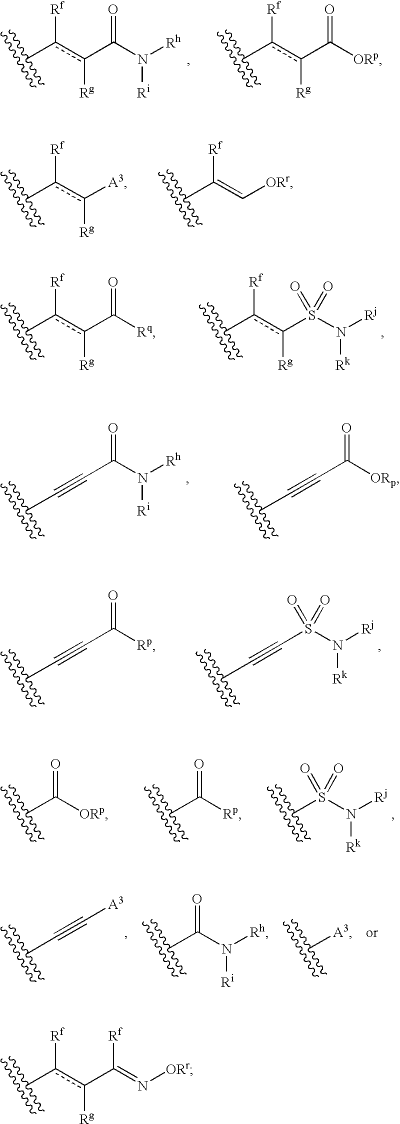

wherein
R^{f}, R^{g}, R^{h}, Rⁱ, R^{j}, and R^{k} are each independently hydrogen, halogen, optionally substituted lower alkyl, optionally substituted lower alkyloxy, optionally substituted lower alkylthio, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, optionally substituted non-aromatic heterocyclic group, or optionally substituted amino;
R^{p}, R^{q}, and R^{r} are each independently hydrogen, optionally substituted lower alkyl, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, or optionally substituted non-aromatic heterocyclic group;
A³ is an optionally substituted aryl or an optionally substituted heteroaryl group;
and with the further proviso that in said compound of Formula (III), Z¹ is not a 3-pyridyl group or a substituted 3-pyridyl group when Z² is —OH, —NH₂, —(C₁-C₆)alkylamino, di(C₁-C₆)alkylamino, an alkyl group, a substituted alkyl group, —(C₃-C₈) cycloalkyl group, a substituted —(C₃-C₈) cycloalkyl group, —(C₃-C₈) cycloalkyl-(C₁-C₆)alkyl group, —O-alkyl group, a substituted —O-alkyl group, —O—(C₃-C₈) cycloalkyl group, a substituted —O—(C₃-C₈) cycloalkyl group, a phenyl group, a fused-phenyl group, a phenyl group substituted with one or more independently selected halogen, —OCH₃, nitro, or dimethyl amino groups, a substituted fused-phenyl group substituted with one or more independently selected halogen, —OCH₃, nitro, or dimethyl amino groups, a —(C₁-C₈) alkyl-phenyl group, —O—(C₀-C₈) alkyl-phenyl group, —(C₃-C₆) alkylene group, —O—(C₃-C₆) alkylene group, —(C₃-C₆) alkynyl group, —O—(C₃-C₆) alkynyl group, —CO(C₁-C₆) alkyl group, —NHCO(C₁-C₆) alkyl group, —NHSO₂(C₁-C₆) alkyl group, —SO₂(C₁-C₆) alkyl group, or —SO₂(C₀-C₆)alkyl-phenyl group;
and with the further proviso that in said compound of Formula (III), Z¹ is not a furyl group or a substituted furyl group when D² and Z² are independently selected from the group consisting of hydrogen and methyl;
and with the further proviso that in said compound comprising structure (III), when D² is —CH₃ and Z² is an optionally substituted phenyl, Z¹ is not selected from the group consisting of —R³³, OR³⁴, and —NR³⁵R³⁶,
wherein
R³³ is hydrogen, optionally substituted (C₃-C₈)cycloalkyl, optionally substituted phenyl, optionally substituted heteroaryl or phenyl-(C₁-C₄) alkyl which is optionally substituted on the phenyl ring, (C₁-C₆)alkyl, (C₂-C₆)alkenyl, (C₂-C₆)alkynyl, the three latter radicals independently being optionally substituted with one or more radicals selected from the group consisting of halogen, (C₁-C₄)alkoxy, (C₁-C₄)alkylthio and NR³⁷R³⁸;
R³⁴ is (C₁-C₆)alkyl, (C₂-C₆)alkenyl, (C₂-C₆)alkynyl, the three latter radicals independently optionally substituted with one or more radicals selected from the group consisting of halogen, (C₁-C₄)alkoxy, (C₁-C₄)alkylthio and NR³⁷R³⁸, or R³⁴ is (C₃-C₆)cycloalkyl optionally substituted with one or more groups selected from the group consisting of halogen, (C₁-C₄)alkyl and (C₁-C₄)alkoxy, and (C₃-C₆)-cycloalkyl-(C₁-C₃)alkyl;
R³⁵ and R³⁶ are hydrogen, (C₁-C₆)alkyl, (C₂-C₆)alkenyl, (C₂-C₆)alkynyl, the 3 latter groups independently optionally substituted by one or more halogen radicals;
R³⁷ is independently selected from the group consisting of hydrogen, (C₁-C₄)alkyl, ((C₁-C₄)alkyl)carbonyl, ((C₁-C₄)alkoxy)carbonyl and CHO;
R³⁸ is independently selected from the group consisting of H and (C₁-C₄)alkyl;
and with the further proviso that in said compound of Formula (III), Z¹ and Z² are not both selected from the group consisting of hydrogen and alkyl;
and with the further proviso that in said compound of Formula (III), Z² is not hydrogen when D¹ and D² are both hydrogen;
and with the further proviso that in said compound of Formula (III), Z¹ is not (C₁-C₆) alkyl, (C₁-C₆) alkoxy, pyridyl, or aryl when D¹ and D² are both hydrogen and when Z² is selected from the group consisting of hydrogen, a (C₁-C₆) alkyl group optionally substituted with 1 to 3 substituents independently selected from the group consisting of hydroxyl, (C₁-C₆) alkoxyl, carboxyl, (C₁-C₆) alkoxycarbonyl, lower alkylthio, fluorine, chlorine, bromine, iodine, amino, mono- or di-substituted amino, phthalimido and nitrogen-containing heterocyclic groups, a (C₃-C₈) cycloalkyl group, a (C₂-C₆) alkenyl group, an arylalkyl group optionally substituted with from 1 to 5 substituents independently selected from the group consisting of (C₁-C₆) alkyl, hydroxyl, (C₁-C₆) alkoxy, halogen, nitro, and amino groups, an aryl group optionally substituted with from 1 to 5 independently selected substituents selected from the group consisting of (C₁-C₆) alkyl, hydroxyl, (C₁-C₆) alkoxy, halogen, nitro, and amino groups, a substituted or unsubstituted heterocyclic group, a (C₁-C₆) alkanoyl group, a benzoyl or a naphthoyl group either of which is optionally substituted with from 1 to 5 independently selected substituents selected from the group consisting of (C₁-C₆) alkyl, hydroxyl, (C₁-C₆) alkoxy, halogen, nitro, and amino groups, a pyridylcarbonyl group, a (C₁-C₆) alkoxycarbonyl group or an amino group optionally substituted with (C₁-C₆) alkyl, arylalkyl having 7 to 15 carbon atoms, substituted or unsubstituted aryl, (C₁-C₆) alkanoyl, optionally substituted aroyl, and (C₁-C₆) alkylidene groups;
and with the further proviso that in said compound of Formula (III), Z¹ is not —O—(CH₂)ₜ—CH₃, wherein t is 0-4 and Z² is halophenyl;
and with the further proviso that in said compound of Formula (III), Z¹ is not a substituted pyridyl, pyrimidinyl, pyrazinyl, pyridazinyl group substituted with —N(R²¹)-(optionally substituted phenyl), wherein said R²¹ is hydrogen, aryl, formyl, (C₁-C₆)alkylcarbonyl, (C₁-C₆)alkyl, (C₁-C₆)alkyloxycarbonyl, (C₁-C₆)alkyl substituted with formyl, (C₁-C₆)alkylcarbonyl, (C₁-C₆)alkyloxycarbonyl, (C₁-C₆)alkylcarbonyloxy, or (C₁-C₆)alkyloxy(C₁-C₆)alkylcarbonyl optionally substituted with (C₁-C₆)alkyloxycarbonyl;
and with the further proviso that in said compound of Formula (III), Z¹ is not a 3-(4-trifluoromethyl-pyridinyl) group or a 3-(4-trifluoromethyl-pyridinyl N-oxide) group;
and with the further proviso that in said compound of Formula (III), Z¹ is not a substituted or unsubstituted group selected from the group consisting of

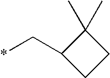

and a —(CH₂)ₜ-adamantanyl group, wherein t is 0-4;
and with the further proviso that in said compound of Formula (III), Z² is not a

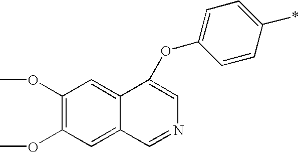

group, which is unsubstituted, or which is substituted with one or more substituents independently selected from the group consisting of halogens and alkyl groups;
and with the further proviso that in said compound of Formula (III), Z² is not a substituted or unsubstituted group selected from the group consisting of:

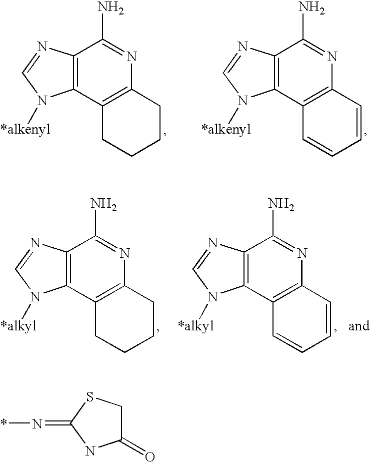

and with the further proviso that said compound of Formula (III), is not a compound set forth in Appendix A, Appendix B, or Appendix C.
95. A method of screening a test compound for replicase complex defect inducer activity comprising:
providing a test compound;
contacting the test compound with a cell expressing an isolated HCV polyprotein that comprises an NS3 protein with a mutation that is at or within about 15 angstroms of C16; and
identifying the test compound as an inducer of an HCV replicase complex defect when the cell is resistant to the test compound.
   96. The method according to claim 95, wherein the cell is an Huh-7 cell.
   97. The method according to claim 95, wherein the isolated HCV polyprotein comprises NS3 protein or fragment thereof.
   98. The method of claim 95, wherein the isolated HCV polyprotein comprises NS3-NS4A or fragment thereof.
   99. The method according to claim 95, wherein the mutation that is at or within about 15 angstroms of C16 is an A39V mutation.
   100. The method according to claim 95, wherein the mutation that is at or within about 15 angstroms of C16 is a C16S mutation.
   101. The method according to claim 95, wherein determining resistance to the test compound comprises determining the EC₅₀ or the EC₉₀ of the test compound.
   102. The method according to claim 101, wherein determining the EC₅₀ or the EC₉₀ is done by performing an RNA dot blot protocol.
   103. The method according to claim 95, wherein the test compound has not previously been screened for induction of an HCV replicase complex defect.
   104. The method according to claim 95, wherein the test compound has not previously been identified as an inducer of an HCV replicase complex defect.
   105. An inducer of a replicase complex defect identified by the method of claim 95, wherein said inducer is a compound of Formula (III)

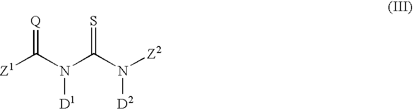

wherein
Q is oxygen or sulfur;
D¹ and D² are independently selected from the group consisting of hydrogen and methyl;
with the proviso that said compound of Formula (III) is not a compound of the formula (III-a):

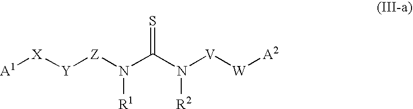

wherein

A₁ and A₂ are independently optionally substituted C₁-C₁₂ alkyl, optionally substituted mono- or di-(C₁-C₈ alkyl)amino, optionally substituted C₂-C₁₂ alkenyl, optionally substituted C₃-C₈ cycloalkyl, an optionally substituted partially unsaturated or aromatic carbocyclic group, or an optionally substituted saturated, partially unsaturated, or aromatic heterocyclic group, wherein at least one of A₁ and A₂ is an optionally substituted aromatic carbocyclic group or an optionally substituted aromatic heterocyclic group;

X and W are independently O, S, NR, or absent, wherein R is hydrogen, optionally substituted (C₁-C₆)alkyl, or optionally substituted aryl(C₀-C₄)alkyl;

V is (C₁-C₆)alkyl, (C₂-C₆)alkenyl, (C₃-C₇)cycloalkyl, or absent, wherein when V is absent, W is absent; and

Y is (C₁-C₆) alkyl, (C₁-C₆)alkyl substituted with (C₃-C₇)cycloalkyl, (C₂-C₆) alkenyl, (C₃-C₇)cycloalkyl, or absent; and

Z is carbonyl, thiocarbonyl, imino, or C₁-C₆ alkylimino; and

R₁ and R₂ are independently hydrogen or methyl;

and with the further proviso that said compound of Formula (III) is not a compound of the formula (III-b):

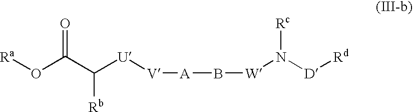

wherein
R^{a} is hydrogen, a substituted or unsubstituted alkyl or cycloalkyl group, a substituted or unsubstituted aryl group or a saturated or unsaturated, optionally substituted heterocyclic group;
R^{b} is hydrogen, a substituted or unsubstituted alkyl or cycloalkyl group, a substituted or unsubstituted aryl group, a saturated or unsaturated, optionally substituted heterocyclic group, an optionally substituted alkenyl group, an optionally substituted alkinyl group, —NR^{2′}SO₂R^{2″}, —NR^{2′}COOR^{2′}, —NR^{2′}COR^{2′}, —NR^{2′}CON(R^{2′})₂, or —N R^{2′}CSN(R^{2′})₂;
R^{2′} is hydrogen, a substituted or unsubstituted alkyl or cycloalkyl group, a substituted or unsubstituted aryl group or a saturated or unsaturated, optionally substituted heterocyclic group;
R^{2″} is a substituted or unsubstituted alkyl, alkenyl or cycloalkyl group, a substituted or unsubstituted aryl group, or a saturated or unsaturated, optionally substituted heterocyclic group;
U′ is a direct bond or a substituted or unsubstituted alkylene group;
V′ is a substituted or unsubstituted alkylene group, —NR^{2′}CO—, or —NR^{2′}SO₂—;
A and B are each independently an optionally substituted 1,3- or 1,4-bridging phenylene group or an optionally substituted 2,4- or 2,5-bridging thienylene group;
W′ is a direct bond or an optionally substituted alkylene group;
D′ is a direct bond or

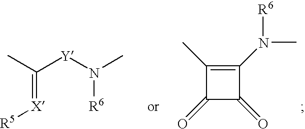

R^{c} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{d}, Y′, R⁵, or R⁶, if present, with formation of an optionally substituted heterocyclic ring system which includes the nitrogen atom to which R^{c} is bonded, and may be saturated or unsaturated and may contain further heteroatoms;
R^{d} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{c}, Y, R⁵ or R⁶, if present, with formation of an optionally substituted heterocyclic ring system which includes the nitrogen atom to which R^{d} is bonded and may be saturated or unsaturated and may contain further heteroatoms;

### X′ is CHNO₂, CHCN, O, N or S;

Y′ is a direct bond or an optionally substituted alkylene or alkine group;
R⁵ is absent, or is hydrogen, a substituted or unsubstituted alkyl or cycloalkyl group, —NO₂, —CN, —COR^{5′}, —COOR^{5′}, or is connected to one of R^{c}, Y′, R^{d}, or R⁶, if present, with formation of an optionally substituted carbocyclic or heterocyclic ring system which includes X′ and can be saturated or unsaturated and may optionally contain one or more additional heteroatoms;
R^{5′} is hydrogen, a substituted or unsubstituted alkyl or cycloalkyl group, a substituted or unsubstituted aryl group or a saturated or unsaturated, optionally substituted heterocyclic group which may optionally contain one or more additional heteroatoms;
R⁶ is a arylcarbonyl group, or a heteroarylcarbonyl group;
and wherein if A is a phenylene group and V′ is —NR^{2′}CO— or —NR^{2′} SO₂—, D′ is not a direct bond and X′ is not N;
and with the further proviso that said compound of Formula (III) is not a compound of the formula (III-c)

X^{1″}—Y^{1″}-Z^{1″}-W^{1″} (III-c)

wherein
X^{1″} is optionally substituted aryl, optionally substituted (C₁-C₈)alkyl-aryl, optionally substituted heteroaryl, or optionally substituted heteroarylalkyl;
Y^{1″} is —NR^{A}CSNR^{A}CO—, wherein R^{A} is independently hydrogen or lower alkyl;
Z^{1″} is optionally substituted phenylene, optionally substituted monocyclic heteroarylene, optionally substituted monocyclic non-aromatic heterocycle-diyl, or optionally substituted monocyclic cycloalkane-diyl;
W^{1″} is a group represented by the formula:

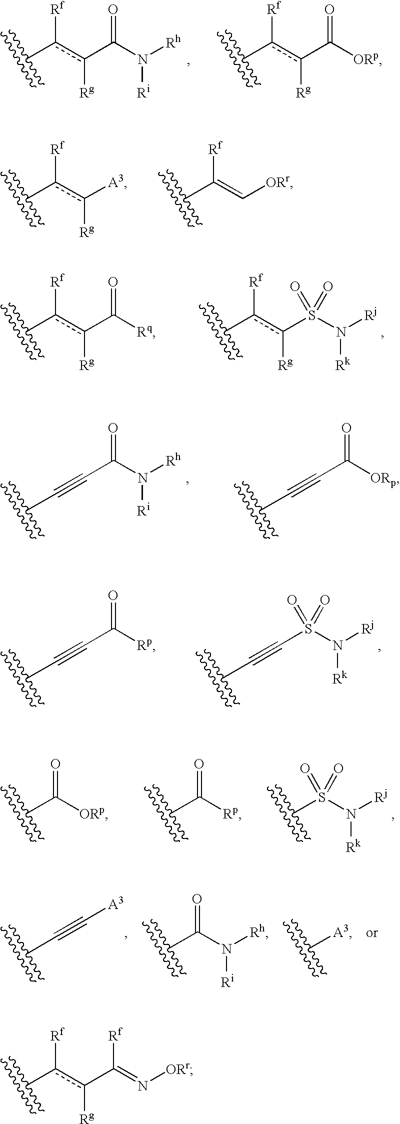

wherein
R^{f}, R^{g}, R^{h}, Rⁱ, R^{j}, and R^{k} are each independently hydrogen, halogen, optionally substituted lower alkyl, optionally substituted lower alkyloxy, optionally substituted lower alkylthio, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, optionally substituted non-aromatic heterocyclic group, or optionally substituted amino;
R^{p}, R^{q}, and R^{r} are each independently hydrogen, optionally substituted lower alkyl, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, or optionally substituted non-aromatic heterocyclic group;
A³ is an optionally substituted aryl or an optionally substituted heteroaryl group;
and with the further proviso that in said compound of Formula (III), Z¹ is not a 3-pyridyl group or a substituted 3-pyridyl group when Z² is —OH, —NH₂, —(C₁-C₆)alkylamino, di(C₁-C₆)alkylamino, an alkyl group, a substituted alkyl group, —(C₃-C₈) cycloalkyl group, a substituted —(C₃-C₈) cycloalkyl group, —(C₃-C₈) cycloalkyl-(C₁-C₆)alkyl group, —O-alkyl group, a substituted —O-alkyl group, —O—(C₃-C₈) cycloalkyl group, a substituted —O—(C₃-C₈) cycloalkyl group, a phenyl group, a fused-phenyl group, a phenyl group substituted with one or more independently selected halogen, —OCH₃, nitro, or dimethyl amino groups, a substituted fused-phenyl group substituted with one or more independently selected halogen, —OCH₃, nitro, or dimethyl amino groups, a —(C₁-C₈) alkyl-phenyl group, —O—(C₀-C₈) alkyl-phenyl group, —(C₃-C₆) alkylene group, —O—(C₃-C₆) alkylene group, —(C₃-C₆) alkynyl group, —O—(C₃-C₆) alkynyl group, —CO(C₁-C₆) alkyl group, —NHCO(C₁-C₆) alkyl group, —NHSO₂(C₁-C₆) alkyl group, —SO₂(C₁-C₆) alkyl group, or —SO₂(C₀-C₆)alkyl-phenyl group;
and with the further proviso that in said compound of Formula (III), Z¹ is not a furyl group or a substituted furyl group when D² and Z² are independently selected from the group consisting of hydrogen and methyl;
and with the further proviso that in said compound comprising structure (III), when D² is —CH₃ and Z² is an optionally substituted phenyl, Z¹ is not selected from the group consisting of —R³³, OR³⁴, and —NR³⁵R³⁶,
wherein
R³³ is hydrogen, optionally substituted (C₃-C₈)cycloalkyl, optionally substituted phenyl, optionally substituted heteroaryl or phenyl-(C₁-C₄) alkyl which is optionally substituted on the phenyl ring, (C₁-C₆)alkyl, (C₂-C₆)alkenyl, (C₂-C₆)alkynyl, the three latter radicals independently being optionally substituted with one or more radicals selected from the group consisting of halogen, (C₁-C₄)alkoxy, (C₁-C₄)alkylthio and NR³⁷R³⁸;
R³⁴ is (C₁-C₆)alkyl, (C₂-C₆)alkenyl, (C₂-C₆)alkynyl, the three latter radicals independently optionally substituted with one or more radicals selected from the group consisting of halogen, (C₁-C₄)alkoxy, (C₁-C₄)alkylthio and NR³⁷R³⁸, or R³⁴ is (C₃-C₆)cycloalkyl optionally substituted with one or more groups selected from the group consisting of halogen, (C₁-C₄)alkyl and (C₁-C₄)alkoxy, and (C₃-C₆)-cycloalkyl-(C₁-C₃)alkyl;
R³⁵ and R³⁶ are hydrogen, (C₁-C₆)alkyl, (C₂-C₆)alkenyl, (C₂-C₆)alkynyl, the 3 latter groups independently optionally substituted by one or more halogen radicals;
R³⁷ is independently selected from the group consisting of hydrogen, (C₁-C₄)alkyl, ((C₁-C₄)alkyl)carbonyl, ((C₁-C₄)alkoxy)carbonyl and CHO;
R³⁸ is independently selected from the group consisting of H and (C₁-C₄)alkyl;
and with the further proviso that in said compound of Formula (III), Z¹ and Z² are not both selected from the group consisting of hydrogen and alkyl;
and with the further proviso that in said compound of Formula (III), Z² is not hydrogen when D¹ and D² are both hydrogen;
and with the further proviso that in said compound of Formula (III), Z¹ is not (C₁-C₆) alkyl, (C₁-C₆) alkoxy, pyridyl, or aryl when D¹ and D² are both hydrogen and when Z² is selected from the group consisting of hydrogen, a (C₁-C₆) alkyl group optionally substituted with 1 to 3 substituents independently selected from the group consisting of hydroxyl, (C₁-C₆) alkoxyl, carboxyl, (C₁-C₆) alkoxycarbonyl, lower alkylthio, fluorine, chlorine, bromine, iodine, amino, mono- or di-substituted amino, phthalimido and nitrogen-containing heterocyclic groups, a (C₃-C₈) cycloalkyl group, a (C₂-C₆) alkenyl group, an arylalkyl group optionally substituted with from 1 to 5 substituents independently selected from the group consisting of (C₁-C₆) alkyl, hydroxyl, (C₁-C₆) alkoxy, halogen, nitro, and amino groups, an aryl group optionally substituted with from 1 to 5 independently selected substituents selected from the group consisting of (C₁-C₆) alkyl, hydroxyl, (C₁-C₆) alkoxy, halogen, nitro, and amino groups, a substituted or unsubstituted heterocyclic group, a (C₁-C₆) alkanoyl group, a benzoyl or a naphthoyl group either of which is optionally substituted with from 1 to 5 independently selected substituents selected from the group consisting of (C₁-C₆) alkyl, hydroxyl, (C₁-C₆) alkoxy, halogen, nitro, and amino groups, a pyridylcarbonyl group, a (C₁-C₆) alkoxycarbonyl group or an amino group optionally substituted with (C₁-C₆) alkyl, arylalkyl having 7 to 15 carbon atoms, substituted or unsubstituted aryl, (C₁-C₆) alkanoyl, optionally substituted aroyl, and (C₁-C₆) alkylidene groups;
and with the further proviso that in said compound of Formula (III), Z¹ is not —O—(CH₂)ₜ—CH₃, wherein t is 0-4 and Z² is halophenyl;
and with the further proviso that in said compound of Formula (III), Z¹ is not a substituted pyridyl, pyrimidinyl, pyrazinyl, pyridazinyl group substituted with —N(R²¹)-(optionally substituted phenyl), wherein said R²¹ is hydrogen, aryl, formyl, (C₁-C₆)alkylcarbonyl, (C₁-C₆)alkyl, (C₁-C₆)alkyloxycarbonyl, (C₁-C₆)alkyl substituted with formyl, (C₁-C₆)alkylcarbonyl, (C₁-C₆)alkyloxycarbonyl, (C₁-C₆)alkylcarbonyloxy, or (C₁-C₆)alkyloxy(C₁-C₆)alkylcarbonyl optionally substituted with (C₁-C₆)alkyloxycarbonyl;
and with the further proviso that in said compound of Formula (II), Z¹ is not a 3-(4-trifluoromethyl-pyridinyl) group or a 3-(4-trifluoromethyl-pyridinyl N-oxide) group;
and with the further proviso that in said compound of Formula (III), Z¹ is not a substituted or unsubstituted group selected from the group consisting of

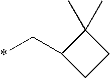

and a —(CH₂)ₜ-adamantanyl group, wherein t is 0-4;
and with the further proviso that in said compound of Formula (III), Z² is not a

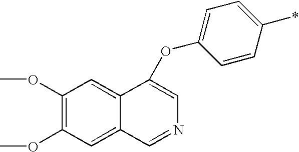

group, which is unsubstituted, or which is substituted with one or more substituents independently selected from the group consisting of halogens and alkyl groups;
and with the further proviso that in said compound of Formula (III), Z² is not a substituted or unsubstituted group selected from the group consisting of:

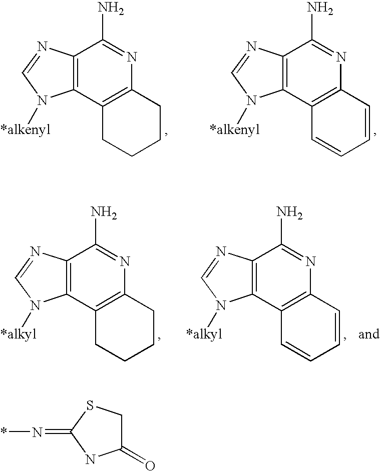

and with the further proviso that said compound of Formula (III), is not a compound set forth in Appendix A, Appendix B, or Appendix C.
106. A method of screening a test compound for replicase complex defect inducer activity comprising:

providing a test compound;

contacting the test compound with a cell that expresses an HCV replicon; and

identifying the test compound as an inducer of an HCV replicase complex defect.

107. The method according to claim 106, wherein the cell is an Huh-7 cell.
108. The method according to claim 106, wherein the HCV replicon is a Con-1 replicon.
109. The method according to claim 106, wherein the test compound has not previously been screened for inhibition of HCV replicase complex assembly.
110. The method according to claim 106, wherein the test compound has not previously been identified as an inducer of an HCV replicase complex defect.
111. The method according to claim 106, wherein the test compound is identified as an inducer of an HCV replicase complex defect by production of a miscleaved nonstructural protein product.
112. The method according to claim 111, wherein the miscleaved nonstructural protein product comprises an N-terminus comprising a portion of NS3 and a C-terminus comprising at least a portion of NS4A.
113. The method according to claim 111, wherein the miscleaved nonstructural protein product comprises an amino terminus comprising greater than or equal to about 1 amino acid of NS3 and a C-terminus comprising at least a portion of NS4A.
114. The method according to claim 111, wherein the miscleaved nonstructural protein product comprises about 20 to about 100 amino acids of NS3 and a C-terminus comprising at least a portion of NS4A.
115. The method according to claim 111, wherein the miscleaved nonstructural protein product comprises an NS4A*.
116. An inducer of a replicase complex defect identified by the method of claim 106, wherein said inducer is a compound of Formula (III)

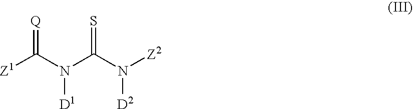

wherein
Q is oxygen or sulfur;
D¹ and D² are independently selected from the group consisting of hydrogen and methyl;
with the proviso that said compound of Formula (III) is not a compound of the formula (III-a):

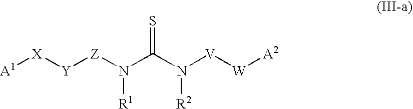

wherein

A₁ and A₂ are independently optionally substituted C₁-C₁₂ alkyl, optionally substituted mono- or di-(C₁-C₈ alkyl)amino, optionally substituted C₂-C₁₂ alkenyl, optionally substituted C₃-C₈ cycloalkyl, an optionally substituted partially unsaturated or aromatic carbocyclic group, or an optionally substituted saturated, partially unsaturated, or aromatic heterocyclic group, wherein at least one of A₁ and A₂ is an optionally substituted aromatic carbocyclic group or an optionally substituted aromatic heterocyclic group;

X and W are independently O, S, NR, or absent, wherein R is hydrogen, optionally substituted (C₁-C₆)alkyl, or optionally substituted aryl(C₀-C₄)alkyl;

V is (C₁-C₆)alkyl, (C₂-C₆)alkenyl, (C₃-C₇)cycloalkyl, or absent, wherein when V is absent, W is absent; and

Y is (C₁-C₆) alkyl, (C₁-C₆)alkyl substituted with (C₃-C₇)cycloalkyl, (C₂-C₆) alkenyl, (C₃-C₇)cycloalkyl, or absent; and

Z is carbonyl, thiocarbonyl, imino, or C₁-C₆ alkylimino; and

R₁ and R₂ are independently hydrogen or methyl;

and with the further proviso that said compound of Formula (III) is not a compound of the formula (III-b):

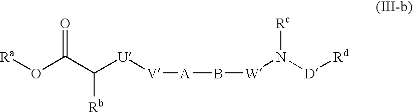

wherein
R^{a} is hydrogen, a substituted or unsubstituted alkyl or cycloalkyl group, a substituted or unsubstituted aryl group or a saturated or unsaturated, optionally substituted heterocyclic group;
R^{b} is hydrogen, a substituted or unsubstituted alkyl or cycloalkyl group, a substituted or unsubstituted aryl group, a saturated or unsaturated, optionally substituted heterocyclic group, an optionally substituted alkenyl group, an optionally substituted alkinyl group, —NR^{2′}SO₂R^{2″}, —NR^{2′}COOR^{2′}, —NR^{2′}COR^{2′}, —NR^{2′}CON(R^{2′})₂, or —N R^{2′}CSN(R^{2′})₂;
R^{2′} is hydrogen, a substituted or unsubstituted alkyl or cycloalkyl group, a substituted or unsubstituted aryl group or a saturated or unsaturated, optionally substituted heterocyclic group;
R^{2″} is a substituted or unsubstituted alkyl, alkenyl or cycloalkyl group, a substituted or unsubstituted aryl group, or a saturated or unsaturated, optionally substituted heterocyclic group;
U′ is a direct bond or a substituted or unsubstituted alkylene group;
V′ is a substituted or unsubstituted alkylene group, —NR^{2′}CO—, or —NR^{2′}SO₂—;
A and B are each independently an optionally substituted 1,3- or 1,4-bridging phenylene group or an optionally substituted 2,4- or 2,5-bridging thienylene group;
W′ is a direct bond or an optionally substituted alkylene group;
D′ is a direct bond or

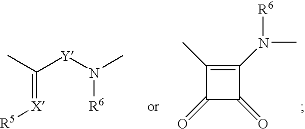

R^{c} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{d}, Y′, R⁵, or R⁶, if present, with formation of an optionally substituted heterocyclic ring system which includes the nitrogen atom to which R^{c} is bonded, and may be saturated or unsaturated and may contain further heteroatoms;
R^{d} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{c}, Y, R⁵ or R⁶, if present, with formation of an optionally substituted heterocyclic ring system which includes the nitrogen atom to which R^{d} is bonded and may be saturated or unsaturated and may contain further heteroatoms;

### X′ is CHNO₂, CHCN, O, N or S;

Y′ is a direct bond or an optionally substituted alkylene or alkine group;
R⁵ is absent, or is hydrogen, a substituted or unsubstituted alkyl or cycloalkyl group, —NO₂, —CN, —COR^{5′}, —COOR^{5′}, or is connected to one of R^{c}, Y′, R^{d}, or R⁶, if present, with formation of an optionally substituted carbocyclic or heterocyclic ring system which includes X′ and can be saturated or unsaturated and may optionally contain one or more additional heteroatoms;
R^{5′} is hydrogen, a substituted or unsubstituted alkyl or cycloalkyl group, a substituted or unsubstituted aryl group or a saturated or unsaturated, optionally substituted heterocyclic group which may optionally contain one or more additional heteroatoms;
R⁶ is a arylcarbonyl group, or a heteroarylcarbonyl group;
and wherein if A is a phenylene group and V′ is —NR^{2′}CO— or —NR^{2′}SO₂—, D′ is not a direct bond and X′ is not N;
and with the further proviso that said compound of Formula (III) is not a compound of the formula (III-c)

X^{1″}—Y^{1″}-Z^{1″}-W^{1″} (III-c)

wherein
X^{1″} is optionally substituted aryl, optionally substituted (C₁-C₈)alkyl-aryl, optionally substituted heteroaryl, or optionally substituted heteroarylalkyl;
Y^{1″} is —NR^{A}CSNR^{A}CO—, wherein R^{A} is independently hydrogen or lower alkyl;
Z^{1″} is optionally substituted phenylene, optionally substituted monocyclic heteroarylene, optionally substituted monocyclic non-aromatic heterocycle-diyl, or optionally substituted monocyclic cycloalkane-diyl;
W^{1″} is a group represented by the formula:

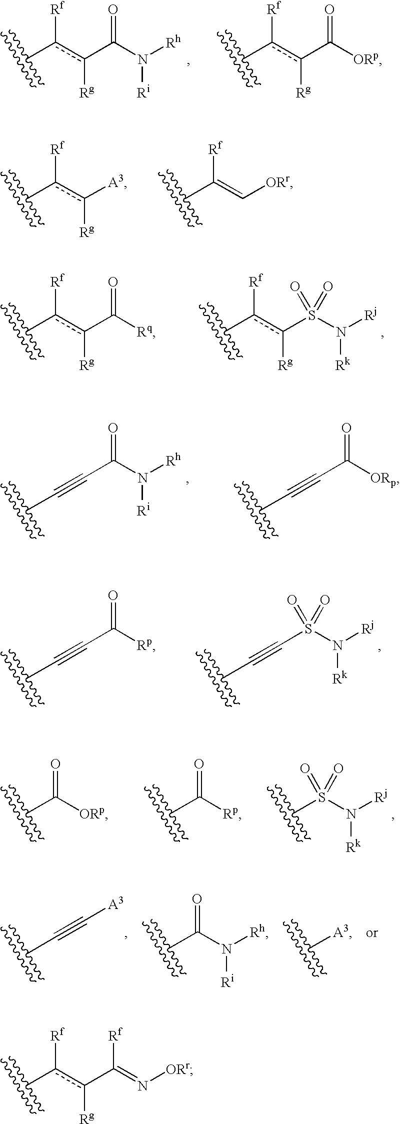

wherein
R^{f}, R^{g}, R^{h}, Rⁱ, R^{j}, and R^{k} are each independently hydrogen, halogen, optionally substituted lower alkyl, optionally substituted lower alkyloxy, optionally substituted lower alkylthio, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, optionally substituted non-aromatic heterocyclic group, or optionally substituted amino;
R^{p}, R^{q}, and R^{r} are each independently hydrogen, optionally substituted lower alkyl, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, or optionally substituted non-aromatic heterocyclic group;
A³ is an optionally substituted aryl or an optionally substituted heteroaryl group;
and with the further proviso that in said compound of Formula (III), Z¹ is not a 3-pyridyl group or a substituted 3-pyridyl group when Z² is —OH, —NH₂, —(C₁-C₆)alkylamino, di(C₁-C₆)alkylamino, an alkyl group, a substituted alkyl group, —(C₃-C₈) cycloalkyl group, a substituted —(C₃-C₈) cycloalkyl group, —(C₃-C₈) cycloalkyl-(C₁-C₆)alkyl group, —O-alkyl group, a substituted —O-alkyl group, —O—(C₃-C₈) cycloalkyl group, a substituted —O—(C₃-C₈) cycloalkyl group, a phenyl group, a fused-phenyl group, a phenyl group substituted with one or more independently selected halogen, —OCH₃, nitro, or dimethyl amino groups, a substituted fused-phenyl group substituted with one or more independently selected halogen, —OCH₃, nitro, or dimethyl amino groups, a —(C₁-C₈) alkyl-phenyl group, —O—(C₀-C₈) alkyl-phenyl group, —(C₃-C₆) alkylene group, —O—(C₃-C₆) alkylene group, —(C₃-C₆) alkynyl group, —O—(C₃-C₆) alkynyl group, —CO(C₁-C₆) alkyl group, —NHCO(C₁-C₆) alkyl group, —NHSO₂(C₁-C₆) alkyl group, —SO₂(C₁-C₆) alkyl group, or —SO₂(C₀-C₆)alkyl-phenyl group;
and with the further proviso that in said compound of Formula (III), Z¹ is not a furyl group or a substituted furyl group when D² and Z² are independently selected from the group consisting of hydrogen and methyl;
and with the further proviso that in said compound comprising structure (III), when D² is —CH₃ and Z² is an optionally substituted phenyl, Z¹ is not selected from the group consisting of —R³³, OR³⁴, and —NR³⁵R³⁶,
wherein
R³³ is hydrogen, optionally substituted (C₃-C₈)cycloalkyl, optionally substituted phenyl, optionally substituted heteroaryl or phenyl-(C₁-C₄) alkyl which is optionally substituted on the phenyl ring, (C₁-C₆)alkyl, (C₂-C₆)alkenyl, (C₂-C₆)alkynyl, the three latter radicals independently being optionally substituted with one or more radicals selected from the group consisting of halogen, (C₁-C₄)alkoxy, (C₁-C₄)alkylthio and NR³⁷R³⁸;
R³⁴ is (C₁-C₆)alkyl, (C₂-C₆)alkenyl, (C₂-C₆)alkynyl, the three latter radicals independently optionally substituted with one or more radicals selected from the group consisting of halogen, (C₁-C₄)alkoxy, (C₁-C₄)alkylthio and NR³⁷R³⁸, or R³⁴ is (C₃-C₆)cycloalkyl optionally substituted with one or more groups selected from the group consisting of halogen, (C₁-C₄)alkyl and (C₁-C₄)alkoxy, and (C₃-C₆)-cycloalkyl-(C₁-C₃)alkyl;
R³⁵ and R³⁶ are hydrogen, (C₁-C₆)alkyl, (C₂-C₆)alkenyl, (C₂-C₆)alkynyl, the 3 latter groups independently optionally substituted by one or more halogen radicals;
R³⁷ is independently selected from the group consisting of hydrogen, (C₁-C₄)alkyl, ((C₁-C₄)alkyl)carbonyl, ((C₁-C₄)alkoxy)carbonyl and CHO;
R³⁸ is independently selected from the group consisting of H and (C₁-C₄)alkyl;
and with the further proviso that in said compound of Formula (III), Z¹ and Z² are not both selected from the group consisting of hydrogen and alkyl;
and with the further proviso that in said compound of Formula (III), Z² is not hydrogen when D¹ and D² are both hydrogen;
and with the further proviso that in said compound of Formula (III), Z¹ is not (C₁-C₆) alkyl, (C₁-C₆) alkoxy, pyridyl, or aryl when D¹ and D² are both hydrogen and when Z² is selected from the group consisting of hydrogen, a (C₁-C₆) alkyl group optionally substituted with 1 to 3 substituents independently selected from the group consisting of hydroxyl, (C₁-C₆) alkoxyl, carboxyl, (C₁-C₆) alkoxycarbonyl, lower alkylthio, fluorine, chlorine, bromine, iodine, amino, mono- or di-substituted amino, phthalimido and nitrogen-containing heterocyclic groups, a (C₃-C₈) cycloalkyl group, a (C₂-C₆) alkenyl group, an arylalkyl group optionally substituted with from 1 to 5 substituents independently selected from the group consisting of (C₁-C₆) alkyl, hydroxyl, (C₁-C₆) alkoxy, halogen, nitro, and amino groups, an aryl group optionally substituted with from 1 to 5 independently selected substituents selected from the group consisting of (C₁-C₆) alkyl, hydroxyl, (C₁-C₆) alkoxy, halogen, nitro, and amino groups, a substituted or unsubstituted heterocyclic group, a (C₁-C₆) alkanoyl group, a benzoyl or a naphthoyl group either of which is optionally substituted with from 1 to 5 independently selected substituents selected from the group consisting of (C₁-C₆) alkyl, hydroxyl, (C₁-C₆) alkoxy, halogen, nitro, and amino groups, a pyridylcarbonyl group, a (C₁-C₆) alkoxycarbonyl group or an amino group optionally substituted with (C₁-C₆) alkyl, arylalkyl having 7 to 15 carbon atoms, substituted or unsubstituted aryl, (C₁-C₆) alkanoyl, optionally substituted aroyl, and (C₁-C₆) alkylidene groups;
and with the further proviso that in said compound of Formula (III), Z¹ is not —O—(CH₂)ₜ—CH₃, wherein t is 0-4 and Z² is halophenyl;
and with the further proviso that in said compound of Formula (III), Z¹ is not a substituted pyridyl, pyrimidinyl, pyrazinyl, pyridazinyl group substituted with —N(R²¹)-(optionally substituted phenyl), wherein said R²¹ is hydrogen, aryl, formyl, (C₁-C₆)alkylcarbonyl, (C₁-C₆)alkyl, (C₁-C₆)alkyloxycarbonyl, (C₁-C₆)alkyl substituted with formyl, (C₁-C₆)alkylcarbonyl, (C₁-C₆)alkyloxycarbonyl, (C₁-C₆)alkylcarbonyloxy, or (C₁-C₆)alkyloxy(C₁-C₆)alkylcarbonyl optionally substituted with (C₁-C₆)alkyloxycarbonyl;
and with the further proviso that in said compound of Formula (III), Z¹ is not a 3-(4-trifluoromethyl-pyridinyl) group or a 3-(4-trifluoromethyl-pyridinyl N-oxide) group;
and with the further proviso that in said compound of Formula (II), Z¹ is not a substituted or unsubstituted group selected from the group consisting of

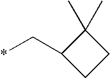

and a —(CH₂)ₜ-adamantanyl group, wherein t is 0-4;
and with the further proviso that in said compound of Formula (III), Z² is not a

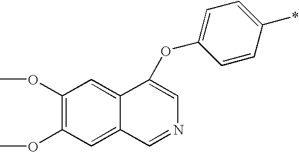

group, which is unsubstituted, or which is substituted with one or more substituents independently selected from the group consisting of halogens and alkyl groups;
and with the further proviso that in said compound of Formula (III), Z² is not a substituted or unsubstituted group selected from the group consisting of:

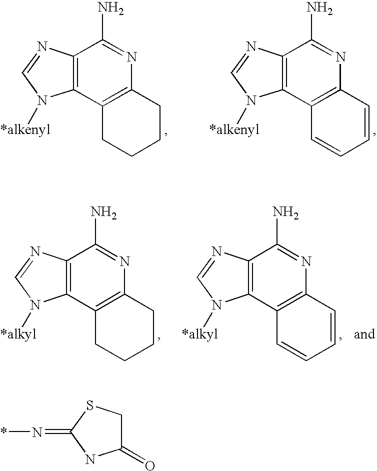

and with the further proviso that said compound of Formula (III), is not a compound set forth in Appendix A, Appendix B, or Appendix C.
117. A method of screening a test compound for replicase complex defect inducer activity comprising:

providing a test compound;

contacting the test compound with a cell that expresses an isolated HCV replicase complex, and

identifying the test compound as an inducer of an HCV replicase complex defect.

118. The method according to claim 117, wherein the cell is an Huh-7 cell.
119. The method according to claim 117, wherein the test compound has not previously been screened for induction of an HCV replicase complex defect.
120. The method according to claim 117, wherein the test compound has not previously been identified as an inducer of an HCV replicase complex defect.
121. The method according to claim 117, wherein the test compound is identified as an inducer of an HCV replicase complex defect by production of a miscleaved nonstructural protein product.
122. The method according to claim 121, wherein the miscleaved nonstructural protein product comprises an N-terminus comprising a portion of NS3 and a C-terminus comprising at least a portion of NS4A.
123. The method according to claim 121, wherein the miscleaved nonstructural protein product comprises an amino terminus comprising greater than or equal to about 1 amino acid of NS3 and a C-terminus comprising at least a portion of NS4A.
124. The method according to claim 121, wherein the miscleaved nonstructural protein product comprises about 20 to about 100 amino acids of NS3 and a C-terminus comprising at least a portion of NS4A.
125. The method according to claim 121, wherein the miscleaved nonstructural protein product comprises an NS4A*.
126. An inducer of a replicase complex defect identified by the method of claim 117, wherein said inducer is a compound of Formula (III)

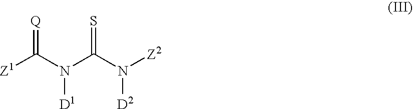

wherein
Q is oxygen or sulfur;
D¹ and D² are independently selected from the group consisting of hydrogen and methyl;
with the proviso that said compound of Formula (III) is not a compound of the formula (III-a):

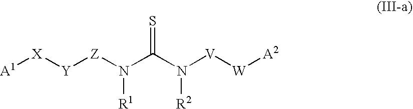

wherein

A₁ and A₂ are independently optionally substituted C₁-C₁₂ alkyl, optionally substituted mono- or di-(C₁-C₈ alkyl)amino, optionally substituted C₂-C₁₂ alkenyl, optionally substituted C₃-C₈ cycloalkyl, an optionally substituted partially unsaturated or aromatic carbocyclic group, or an optionally substituted saturated, partially unsaturated, or aromatic heterocyclic group, wherein at least one of A₁ and A₂ is an optionally substituted aromatic carbocyclic group or an optionally substituted aromatic heterocyclic group;

X and W are independently O, S, NR, or absent, wherein R is hydrogen, optionally substituted (C₁-C₆)alkyl, or optionally substituted aryl(C₀-C₄)alkyl;

V is (C₁-C₆)alkyl, (C₂-C₆)alkenyl, (C₃-C₇)cycloalkyl, or absent, wherein when V is absent, W is absent; and

Y is (C₁-C₆) alkyl, (C₁-C₆)alkyl substituted with (C₃-C₇)cycloalkyl, (C₂-C₆) alkenyl, (C₃-C₇)cycloalkyl, or absent; and

Z is carbonyl, thiocarbonyl, imino, or C₁-C₆ alkylimino; and

R₁ and R₂ are independently hydrogen or methyl;

and with the further proviso that said compound of Formula (III) is not a compound of the formula (III-b):

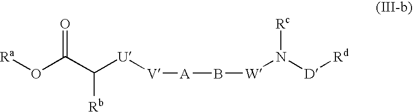

wherein
R^{a} is hydrogen, a substituted or unsubstituted alkyl or cycloalkyl group, a substituted or unsubstituted aryl group or a saturated or unsaturated, optionally substituted heterocyclic group;
R^{b} is hydrogen, a substituted or unsubstituted alkyl or cycloalkyl group, a substituted or unsubstituted aryl group, a saturated or unsaturated, optionally substituted heterocyclic group, an optionally substituted alkenyl group, an optionally substituted alkinyl group, —NR^{2′}SO₂R^{2″}, —NR^{2′}COOR^{2′}, —NR^{2′}COR^{2′}, —NR^{2′}CON(R^{2′})₂, or —N R^{2′}CSN(R^{2′})₂;
R^{2′} is hydrogen, a substituted or unsubstituted alkyl or cycloalkyl group, a substituted or unsubstituted aryl group or a saturated or unsaturated, optionally substituted heterocyclic group;
R^{2″} is a substituted or unsubstituted alkyl, alkenyl or cycloalkyl group, a substituted or unsubstituted aryl group, or a saturated or unsaturated, optionally substituted heterocyclic group;
U′ is a direct bond or a substituted or unsubstituted alkylene group;
V′ is a substituted or unsubstituted alkylene group, —NR^{2′}CO—, or —NR^{2′}SO₂—;
A and B are each independently an optionally substituted 1,3- or 1,4-bridging phenylene group or an optionally substituted 2,4- or 2,5-bridging thienylene group;
W′ is a direct bond or an optionally substituted alkylene group;
D′ is a direct bond or

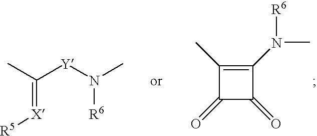

R^{c} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{d}, Y′, R⁵, or R⁶, if present, with formation of an optionally substituted heterocyclic ring system which includes the nitrogen atom to which R^{c} is bonded, and may be saturated or unsaturated and may contain further heteroatoms;
R^{d} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{c}, Y, R⁵ or R⁶, if present, with formation of an optionally substituted heterocyclic ring system which includes the nitrogen atom to which R^{d} is bonded and may be saturated or unsaturated and may contain further heteroatoms;

### X′ is CHNO₂, CHCN, O, N or S;

Y′ is a direct bond or an optionally substituted alkylene or alkine group;
R⁵ is absent, or is hydrogen, a substituted or unsubstituted alkyl or cycloalkyl group, —NO₂, —CN, —COR^{5′}, —COOR^{5′}, or is connected to one of R^{c}, Y′, R^{d}, or R⁶, if present, with formation of an optionally substituted carbocyclic or heterocyclic ring system which includes X′ and can be saturated or unsaturated and may optionally contain one or more additional heteroatoms;
R^{5′} is hydrogen, a substituted or unsubstituted alkyl or cycloalkyl group, a substituted or unsubstituted aryl group or a saturated or unsaturated, optionally substituted heterocyclic group which may optionally contain one or more additional heteroatoms;
R⁶ is a arylcarbonyl group, or a heteroarylcarbonyl group;
and wherein if A is a phenylene group and V′ is —NR^{2′}CO— or —NR^{2′} SO₂—, D′ is not a direct bond and X′ is not N;
and with the further proviso that said compound of Formula (III) is not a compound of the formula (III-c)

X^{1″}—Y^{1″}-Z^{1″}-W^{1″} (III-c)

wherein
X^{1″} is optionally substituted aryl, optionally substituted (C₁-C₈)alkyl-aryl, optionally substituted heteroaryl, or optionally substituted heteroarylalkyl;
Y^{1″} is —NR^{A}CSNR^{A}CO—, wherein R^{A} is independently hydrogen or lower alkyl;
Z^{1″} is optionally substituted phenylene, optionally substituted monocyclic heteroarylene, optionally substituted monocyclic non-aromatic heterocycle-diyl, or optionally substituted monocyclic cycloalkane-diyl;
W^{1″} is a group represented by the formula:

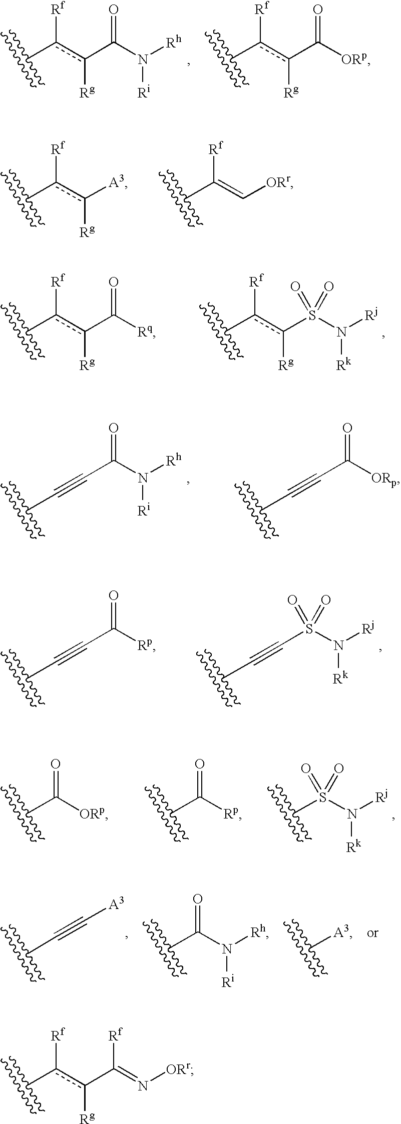

wherein
R^{f}, R^{g}, R^{h}, Rⁱ, R^{j}, and R^{k} are each independently hydrogen, halogen, optionally substituted lower alkyl, optionally substituted lower alkyloxy, optionally substituted lower alkylthio, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, optionally substituted non-aromatic heterocyclic group, or optionally substituted amino;
R^{p}, R^{q}, and R^{r} are each independently hydrogen, optionally substituted lower alkyl, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, or optionally substituted non-aromatic heterocyclic group;
A³ is an optionally substituted aryl or an optionally substituted heteroaryl group;
and with the further proviso that in said compound of Formula (III), Z¹ is not a 3-pyridyl group or a substituted 3-pyridyl group when Z² is —OH, —NH₂, —(C₁-C₆)alkylamino, di(C₁-C₆)alkylamino, an alkyl group, a substituted alkyl group, —(C₃-C₈) cycloalkyl group, a substituted —(C₃-C₈) cycloalkyl group, —(C₃-C₈) cycloalkyl-(C₁-C₆)alkyl group, —O-alkyl group, a substituted —O-alkyl group, —O—(C₃-C₈) cycloalkyl group, a substituted —O—(C₃-C₈) cycloalkyl group, a phenyl group, a fused-phenyl group, a phenyl group substituted with one or more independently selected halogen, —OCH₃, nitro, or dimethyl amino groups, a substituted fused-phenyl group substituted with one or more independently selected halogen, —OCH₃, nitro, or dimethyl amino groups, a —(C₁-C₈) alkyl-phenyl group, —O—(C₀-C₈) alkyl-phenyl group, —(C₃-C₆) alkylene group, —O—(C₃-C₆) alkylene group, —(C₃-C₆) alkynyl group, (C₃-C₆) alkynyl group, —CO(C₁-C₆) alkyl group, —NHCO(C₁-C₆) alkyl group, —NHSO₂(C₁-C₆) alkyl group, —SO₂(C₁-C₆) alkyl group, or —SO₂(C₀-C₆)alkyl-phenyl group;
and with the further proviso that in said compound of Formula (III), Z¹ is not a furyl group or a substituted furyl group when D² and Z² are independently selected from the group consisting of hydrogen and methyl;
and with the further proviso that in said compound comprising structure (III), when D² is —CH₃ and Z² is an optionally substituted phenyl, Z¹ is not selected from the group consisting of —R³³, OR³⁴, and —NR³⁵R³⁶,
wherein
R³³ is hydrogen, optionally substituted (C₃-C₈)cycloalkyl, optionally substituted phenyl, optionally substituted heteroaryl or phenyl-(C₁-C₄) alkyl which is optionally substituted on the phenyl ring, (C₁-C₆)alkyl, (C₂-C₆)alkenyl, (C₂-C₆)alkynyl, the three latter radicals independently being optionally substituted with one or more radicals selected from the group consisting of halogen, (C₁-C₄)alkoxy, (C₁-C₄)alkylthio and R³⁷R³⁸;
R³⁴ is (C₁-C₆)alkyl, (C₂-C₆)alkenyl, (C₂-C₆)alkynyl, the three latter radicals independently optionally substituted with one or more radicals selected from the group consisting of halogen, (C₁-C₄)alkoxy, (C₁-C₄)alkylthio and NR³⁷R³⁸, or R³⁴ is (C₃-C₆)cycloalkyl optionally substituted with one or more groups selected from the group consisting of halogen, (C₁-C₄)alkyl and (C₁-C₄)alkoxy, and (C₃-C₆)-cycloalkyl-(C₁-C₃)alkyl;
R³⁵ and R³⁶ are hydrogen, (C₁-C₆)alkyl, (C₂-C₆)alkenyl, (C₂-C₆)alkynyl, the 3 latter groups independently optionally substituted by one or more halogen radicals;
R³⁷ is independently selected from the group consisting of hydrogen, (C₁-C₄)alkyl, ((C₁-C₄)alkyl)carbonyl, ((C₁-C₄)alkoxy)carbonyl and CHO;
R³⁸ is independently selected from the group consisting of H and (C₁-C₄)alkyl;
and with the further proviso that in said compound of Formula (III), Z¹ and Z² are not both selected from the group consisting of hydrogen and alkyl;
and with the further proviso that in said compound of Formula (III), Z² is not hydrogen when D¹ and D² are both hydrogen;
and with the further proviso that in said compound of Formula (III), Z¹ is not (C₁-C₆) alkyl, (C₁-C₆) alkoxy, pyridyl, or aryl when D¹ and D² are both hydrogen and when Z² is selected from the group consisting of hydrogen, a (C₁-C₆) alkyl group optionally substituted with 1 to 3 substituents independently selected from the group consisting of hydroxyl, (C₁-C₆) alkoxyl, carboxyl, (C₁-C₆) alkoxycarbonyl, lower alkylthio, fluorine, chlorine, bromine, iodine, amino, mono- or di-substituted amino, phthalimido and nitrogen-containing heterocyclic groups, a (C₃-C₈) cycloalkyl group, a (C₂-C₆) alkenyl group, an arylalkyl group optionally substituted with from 1 to 5 substituents independently selected from the group consisting of (C₁-C₆) alkyl, hydroxyl, (C₁-C₆) alkoxy, halogen, nitro, and amino groups, an aryl group optionally substituted with from 1 to 5 independently selected substituents selected from the group consisting of (C₁-C₆) alkyl, hydroxyl, (C₁-C₆) alkoxy, halogen, nitro, and amino groups, a substituted or unsubstituted heterocyclic group, a (C₁-C₆) alkanoyl group, a benzoyl or a naphthoyl group either of which is optionally substituted with from 1 to 5 independently selected substituents selected from the group consisting of (C₁-C₆) alkyl, hydroxyl, (C₁-C₆) alkoxy, halogen, nitro, and amino groups, a pyridylcarbonyl group, a (C₁-C₆) alkoxycarbonyl group or an amino group optionally substituted with (C₁-C₆) alkyl, arylalkyl having 7 to 15 carbon atoms, substituted or unsubstituted aryl, (C₁-C₆) alkanoyl, optionally substituted aroyl, and (C₁-C₆) alkylidene groups;
and with the further proviso that in said compound of Formula (III), Z¹ is not —O—(CH₂)ₜ—CH₃, wherein t is 0-4 and Z² is halophenyl;
and with the further proviso that in said compound of Formula (III), Z¹ is not a substituted pyridyl, pyrimidinyl, pyrazinyl, pyridazinyl group substituted with —N(R²¹)-(optionally substituted phenyl), wherein said R²¹ is hydrogen, aryl, formyl, (C₁-C₆)alkylcarbonyl, (C₁-C₆)alkyl, (C₁-C₆)alkyloxycarbonyl, (C₁-C₆)alkyl substituted with formyl, (C₁-C₆)alkylcarbonyl, (C₁-C₆)alkyloxycarbonyl, (C₁-C₆)alkylcarbonyloxy, or (C₁-C₆)alkyloxy(C₁-C₆)alkylcarbonyl optionally substituted with (C₁-C₆)alkyloxycarbonyl;
and with the further proviso that in said compound of Formula (III), Z¹ is not a 3-(4-trifluoromethyl-pyridinyl) group or a 3-(4-trifluoromethyl-pyridinyl N-oxide) group;
and with the further proviso that in said compound of Formula (III), Z¹ is not a substituted or unsubstituted group selected from the group consisting of

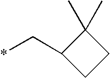

and a —(CH₂)ₜ-adamantanyl group, wherein t is 0-4;
and with the further proviso that in said compound of Formula (III), Z² is not a

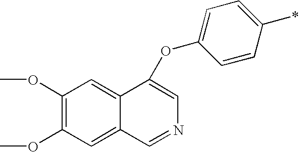

group, which is unsubstituted, or which is substituted with one or more substituents independently selected from the group consisting of halogens and alkyl groups;
and with the further proviso that in said compound of Formula (III), Z² is not a substituted or unsubstituted group selected from the group consisting of:

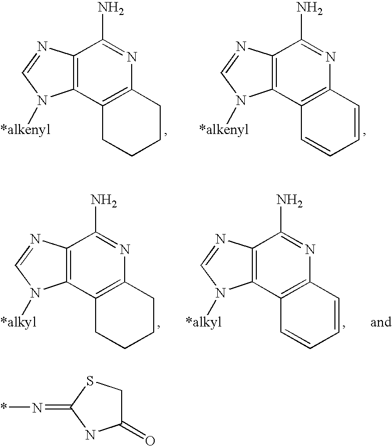

and with the further proviso that said compound of Formula (III), is not a compound set forth in Appendix A, Appendix B, or Appendix C.
127. A method of screening a test compound for replicase complex defect inducer activity comprising:

providing a test compound;

contacting the test compound with a cell that expresses an isolated HCV polyprotein or fragment thereof; and

identifying the test compound as an inducer of an HCV replicase complex defect.

128. The method according to claim 127, wherein the cell is an Huh-7 cell.
129. The method according to claim 127, wherein the HCV polyprotein or fragment thereof comprises an NS3-NS5B polyprotein or fragment thereof.
130. The method according to claim 127, wherein the HCV polyprotein or fragment thereof comprises an NS3-NS4A polyprotein or fragment thereof.
131. The method according to claim 127, wherein the test compound has not previously been screened for induction of an HCV replicase complex defect.
132. The method according to claim 127, wherein the test compound has not previously been identified as an inducer of an HCV replicase complex defect.
133. The method according to claim 127, wherein the test compound is identified as an inducer of an HCV replicase complex defect by production of a miscleaved nonstructural protein product.
134. The method according to claim 133, wherein the miscleaved nonstructural protein product comprises an N-terminus comprising a portion of NS3 and a C-terminus comprising at least a portion of NS4A.
135. The method according to claim 134, wherein the miscleaved nonstructural protein product comprises an amino terminus comprising greater than or equal to about 1 amino acid of NS3 and a C-terminus comprising at least a portion of NS4A.
136. The method according to claim 134, wherein the miscleaved nonstructural protein product comprises about 20 to about 100 amino acids of NS3 and a C-terminus comprising at least a portion of NS4A.
137. The method according to claim 134, wherein the miscleaved nonstructural protein product comprises an NS4A*.
138. An inducer of a replicase complex defect identified by the method of claim 127, wherein said inducer is a compound of Formula (III)

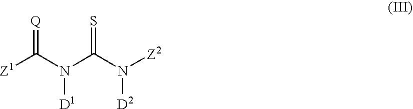

wherein
Q is oxygen or sulfur;
D¹ and D² are independently selected from the group consisting of hydrogen and methyl;
with the proviso that said compound of Formula (III) is not a compound of the formula (III-a):

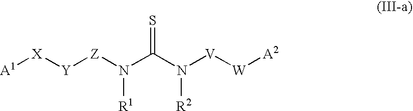

wherein

A₁ and A₂ are independently optionally substituted C₁-C₁₂ alkyl, optionally substituted mono- or di-(C₁-C₈ alkyl)amino, optionally substituted C₂-C₁₂ alkenyl, optionally substituted C₃-C₈ cycloalkyl, an optionally substituted partially unsaturated or aromatic carbocyclic group, or an optionally substituted saturated, partially unsaturated, or aromatic heterocyclic group, wherein at least one of A₁ and A₂ is an optionally substituted aromatic carbocyclic group or an optionally substituted aromatic heterocyclic group;

X and W are independently O, S, NR, or absent, wherein R is hydrogen, optionally substituted (C₁-C₆)alkyl, or optionally substituted aryl(C₀-C₄)alkyl;

V is (C₁-C₆)alkyl, (C₂-C₆)alkenyl, (C₃-C₇)cycloalkyl, or absent, wherein when V is absent, W is absent; and

Y is (C₁-C₆) alkyl, (C₁-C₆)alkyl substituted with (C₃-C₇)cycloalkyl, (C₂-C₆) alkenyl, (C₃-C₇)cycloalkyl, or absent; and

Z is carbonyl, thiocarbonyl, imino, or C₁-C₆ alkylimino; and

R₁ and R₂ are independently hydrogen or methyl;

and with the further proviso that said compound of Formula (III) is not a compound of the formula (III-b):

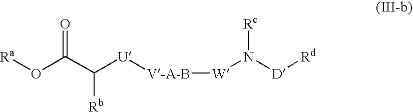

wherein
R^{a} is hydrogen, a substituted or unsubstituted alkyl or cycloalkyl group, a substituted or unsubstituted aryl group or a saturated or unsaturated, optionally substituted heterocyclic group;
R^{b} is hydrogen, a substituted or unsubstituted alkyl or cycloalkyl group, a substituted or unsubstituted aryl group, a saturated or unsaturated, optionally substituted heterocyclic group, an optionally substituted alkenyl group, an optionally substituted alkinyl group, —NR^{2′}SO₂R^{2′}, —NR^{2′}COOR^{2′}, —NR^{2′}COR^{2′}, —NR^{2′}CON(R^{2′})₂ or —N R^{2′}CSN(R^{2′})₂;
R^{2′} is hydrogen, a substituted or unsubstituted alkyl or cycloalkyl group, a substituted or unsubstituted aryl group or a saturated or unsaturated, optionally substituted heterocyclic group;
R^{2″} is a substituted or unsubstituted alkyl, alkenyl or cycloalkyl group, a substituted or unsubstituted aryl group, or a saturated or unsaturated, optionally substituted heterocyclic group;
U′ is a direct bond or a substituted or unsubstituted alkylene group;
V′ is a substituted or unsubstituted alkylene group, —NR^{2′}CO—, or —NR^{2′}SO₂—;
A and B are each independently an optionally substituted 1,3- or 1,4-bridging phenylene group or an optionally substituted 2,4- or 2,5-bridging thienylene group;
W′ is a direct bond or an optionally substituted alkylene group;
D′ is a direct bond or

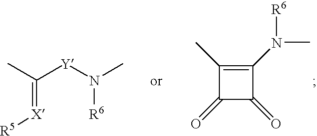

R^{c} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{d}, Y′, R⁵, or R⁶, if present, with formation of an optionally substituted heterocyclic ring system which includes the nitrogen atom to which R^{c} is bonded, and may be saturated or unsaturated and may contain further heteroatoms;
R^{d} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{c}, Y, R⁵ or R⁶, if present, with formation of an optionally substituted heterocyclic ring system which includes the nitrogen atom to which R^{d} is bonded and may be saturated or unsaturated and may contain further heteroatoms;

### X′ is CHNO₂, CHCN, O, N or S;

Y′ is a direct bond or an optionally substituted alkylene or alkine group;
R⁵ is absent, or is hydrogen, a substituted or unsubstituted alkyl or cycloalkyl group, —NO₂, —CN, —COR^{5′}, —COOR^{5′}, or is connected to one of R^{c}, Y′, R^{d}, or R⁶, if present, with formation of an optionally substituted carbocyclic or heterocyclic ring system which includes X′ and can be saturated or unsaturated and may optionally contain one or more additional heteroatoms;
R^{5′} is hydrogen, a substituted or unsubstituted alkyl or cycloalkyl group, a substituted or unsubstituted aryl group or a saturated or unsaturated, optionally substituted heterocyclic group which may optionally contain one or more additional heteroatoms;
R⁶ is a arylcarbonyl group, or a heteroarylcarbonyl group;
and wherein if A is a phenylene group and V′ is —NR^{2′}CO— or —NR² SO₂—, D′ is not a direct bond and X′ is not N;
and with the further proviso that said compound of Formula (III) is not a compound of the formula (III-c)

X^{1″}—Y^{1″}-Z^{1″}-W^{1″} (III-c)

wherein
X^{1″} is optionally substituted aryl, optionally substituted (C₁-C₈)alkyl-aryl, optionally substituted heteroaryl, or optionally substituted heteroarylalkyl;
Y^{1″} is —NR^{A}CSNR^{A}CO—, wherein R^{A} is independently hydrogen or lower alkyl;
Z^{1″} is optionally substituted phenylene, optionally substituted monocyclic heteroarylene, optionally substituted monocyclic non-aromatic heterocycle-diyl, or optionally substituted monocyclic cycloalkane-diyl;
W^{1″} is a group represented by the formula:

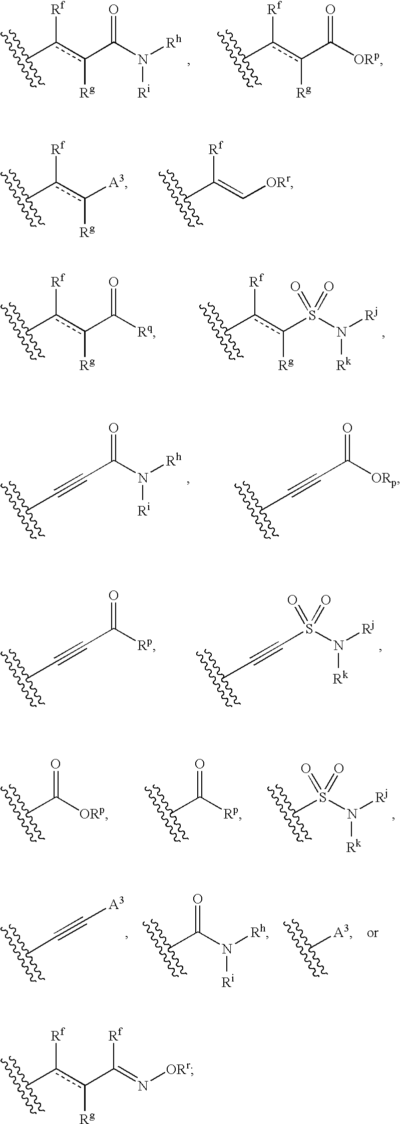

wherein
R^{f}, R^{g}, R^{h}, Rⁱ, R^{j}, and R^{k} are each independently hydrogen, halogen, optionally substituted lower alkyl, optionally substituted lower alkyloxy, optionally substituted lower alkylthio, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, optionally substituted non-aromatic heterocyclic group, or optionally substituted amino;
R^{p}, R^{q}, and R^{r} are each independently hydrogen, optionally substituted lower alkyl, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, or optionally substituted non-aromatic heterocyclic group;
A³ is an optionally substituted aryl or an optionally substituted heteroaryl group;
and with the further proviso that in said compound of Formula (III), Z¹ is not a 3-pyridyl group or a substituted 3-pyridyl group when Z² is —OH, —NH₂, —(C₁-C₆)alkylamino, di(C₁-C₆)alkylamino, an alkyl group, a substituted alkyl group, —(C₃-C₈) cycloalkyl group, a substituted —(C₃-C₈) cycloalkyl group, —(C₃-C₈) cycloalkyl-(C₁-C₆)alkyl group, —O-alkyl group, a substituted —O-alkyl group, —O—(C₃-C₈) cycloalkyl group, a substituted —O—(C₃-C₈) cycloalkyl group, a phenyl group, a fused-phenyl group, a phenyl group substituted with one or more independently selected halogen, —OCH₃, nitro, or dimethyl amino groups, a substituted fused-phenyl group substituted with one or more independently selected halogen, —OCH₃, nitro, or dimethyl amino groups, a —(C₁-C₈) alkyl-phenyl group, —O—(C₀-C₈) alkyl-phenyl group, —(C₃-C₆) alkylene group, —O—(C₃-C₆) alkylene group, —(C₃-C₆) alkynyl group, —O—(C₃-C₆) alkynyl group, —CO(C₁-C₆) alkyl group, —NHCO(C₁-C₆) alkyl group, —NHSO₂(C₁-C₆) alkyl group, —SO₂(C₁-C₆) alkyl group, or —SO₂(C₀-C₆)alkyl-phenyl group;
and with the further proviso that in said compound of Formula (III), Z¹ is not a furyl group or a substituted furyl group when D² and Z² are independently selected from the group consisting of hydrogen and methyl;
and with the further proviso that in said compound comprising structure (III), when D² is —CH₃ and Z² is an optionally substituted phenyl, Z¹ is not selected from the group consisting of —R³³, OR³⁴, and —NR³⁵R³⁶,
wherein
R³³ is hydrogen, optionally substituted (C₃-C₈)cycloalkyl, optionally substituted phenyl, optionally substituted heteroaryl or phenyl-(C₁-C₄) alkyl which is optionally substituted on the phenyl ring, (C₁-C₆)alkyl, (C₂-C₆)alkenyl, (C₂-C₆)alkynyl, the three latter radicals independently being optionally substituted with one or more radicals selected from the group consisting of halogen, (C₁-C₄)alkoxy, (C₁-C₄)alkylthio and NR³⁷R³⁸;
R³⁴ is (C₁-C₆)alkyl, (C₂-C₆)alkenyl, (C₂-C₆)alkynyl, the three latter radicals independently optionally substituted with one or more radicals selected from the group consisting of halogen, (C₁-C₄)alkoxy, (C₁-C₄)alkylthio and NR³⁷R³⁸, or R³⁴ is (C₃-C₆)cycloalkyl optionally substituted with one or more groups selected from the group consisting of halogen, (C₁-C₄)alkyl and (C₁-C₄)alkoxy, and (C₃-C₆)-cycloalkyl-(C₁-C₃)alkyl;
R³⁵ and R³⁶ are hydrogen, (C₁-C₆)alkyl, (C₂-C₆)alkenyl, (C₂-C₆)alkynyl, the 3 latter groups independently optionally substituted by one or more halogen radicals;
R³⁷ is independently selected from the group consisting of hydrogen, (C₁-C₄)alkyl, ((C₁-C₄)alkyl)carbonyl, ((C₁-C₄)alkoxy)carbonyl and CHO;
R³⁸ is independently selected from the group consisting of H and (C₁-C₄)alkyl;
and with the further proviso that in said compound of Formula (III), Z¹ and Z² are not both selected from the group consisting of hydrogen and alkyl;
and with the further proviso that in said compound of Formula (III), Z² is not hydrogen when D¹ and D² are both hydrogen;
and with the further proviso that in said compound of Formula (III), Z¹ is not (C₁-C₆) alkyl, (C₁-C₆) alkoxy, pyridyl, or aryl when D¹ and D² are both hydrogen and when Z² is selected from the group consisting of hydrogen, a (C₁-C₆) alkyl group optionally substituted with 1 to 3 substituents independently selected from the group consisting of hydroxyl, (C₁-C₆) alkoxyl, carboxyl, (C₁-C₆) alkoxycarbonyl, lower alkylthio, fluorine, chlorine, bromine, iodine, amino, mono- or di-substituted amino, phthalimido and nitrogen-containing heterocyclic groups, a (C₃-C₈) cycloalkyl group, a (C₂-C₆) alkenyl group, an arylalkyl group optionally substituted with from 1 to 5 substituents independently selected from the group consisting of (C₁-C₆) alkyl, hydroxyl, (C₁-C₆) alkoxy, halogen, nitro, and amino groups, an aryl group optionally substituted with from 1 to 5 independently selected substituents selected from the group consisting of (C₁-C₆) alkyl, hydroxyl, (C₁-C₆) alkoxy, halogen, nitro, and amino groups, a substituted or unsubstituted heterocyclic group, a (C₁-C₆) alkanoyl group, a benzoyl or a naphthoyl group either of which is optionally substituted with from 1 to 5 independently selected substituents selected from the group consisting of (C₁-C₆) alkyl, hydroxyl, (C₁-C₆) alkoxy, halogen, nitro, and amino groups, a pyridylcarbonyl group, a (C₁-C₆) alkoxycarbonyl group or an amino group optionally substituted with (C₁-C₆) alkyl, arylalkyl having 7 to 15 carbon atoms, substituted or unsubstituted aryl, (C₁-C₆) alkanoyl, optionally substituted aroyl, and (C₁-C₆) alkylidene groups;
and with the further proviso that in said compound of Formula (III), Z¹ is not —O—(CH₂)ₜ—CH₃, wherein t is 0-4 and Z² is halophenyl;
and with the further proviso that in said compound of Formula (III), Z¹ is not a substituted pyridyl, pyrimidinyl, pyrazinyl, pyridazinyl group substituted with —N(R²¹)-(optionally substituted phenyl), wherein said R²¹ is hydrogen, aryl, formyl, (C₁-C₆)alkylcarbonyl, (C₁-C₆)alkyl, (C₁-C₆)alkyloxycarbonyl, (C₁-C₆)alkyl substituted with formyl, (C₁-C₆)alkylcarbonyl, (C₁-C₆)alkyloxycarbonyl, (C₁-C₆)alkylcarbonyloxy, or (C₁-C₆)alkyloxy(C₁-C₆)alkylcarbonyl optionally substituted with (C₁-C₆)alkyloxycarbonyl;
and with the further proviso that in said compound of Formula (III), Z¹ is not a 3-(4-trifluoromethyl-pyridinyl) group or a 3-(4-trifluoromethyl-pyridinyl N-oxide) group;
and with the further proviso that in said compound of Formula (III), Z¹ is not a substituted or unsubstituted group selected from the group consisting of

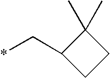

and a —(CH₂)ₜ-adamantanyl group, wherein t is 0-4;
and with the further proviso that in said compound of Formula (III), Z² is not a

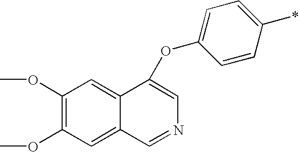

group, which is unsubstituted, or which is substituted with one or more substituents independently selected from the group consisting of halogens and alkyl groups;
and with the further proviso that in said compound of Formula (III), Z² is not a substituted or unsubstituted group selected from the group consisting of:

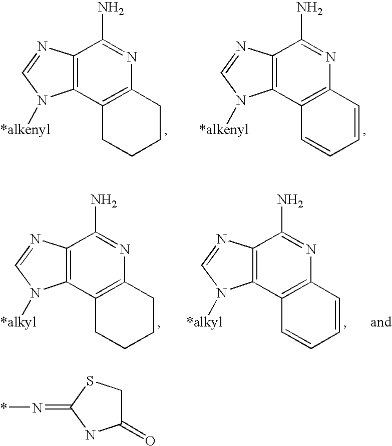

and with the further proviso that said compound of Formula (III), is not a compound set forth in Appendix A, Appendix B, or Appendix C.
139. A method of screening a test compound for replicase complex defect inducer activity comprising:

providing a test compound;

contacting the test compound with an HCV replicon; and

identifying the test compound as an inducer of an HCV replicase complex defect when the level of p14 protein is increased.

140. The method according to claim 139, wherein the HCV replicon is a Con-1 replicon.
141. The method according to claim 139, where the HCV replicon has a mutation in an NS3 protein or fragment thereof.
142. The method according to claim 139, wherein the HCV replicon has a mutation at or within about 15 angstroms of C16 in an NS3 protein or fragment thereof.
143. The method according to claim 139, wherein the HCV replicon has an A39V mutation in an NS3 protein or fragment thereof.
144. The method according to claim 139, wherein the HCV replicon has an C16S mutation in an NS3 protein or fragment thereof.
145. The method according to claim 139, wherein the level of NS3 production is reduced.
146. An inducer of a replicase complex defect identified by the method of claim 139, wherein said inducer is a compound of Formula (III)

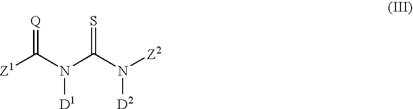

wherein
Q is oxygen or sulfur;
D¹ and D² are independently selected from the group consisting of hydrogen and methyl;
with the proviso that said compound of Formula (III) is not a compound of the formula (III-a):

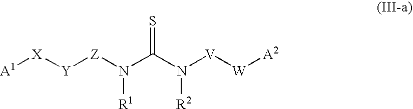

wherein

A₁ and A₂ are independently optionally substituted C₁-C₁₂ alkyl, optionally substituted mono- or di-(C₁-C₈ alkyl)amino, optionally substituted C₂-C₁₂ alkenyl, optionally substituted C₃-C₈ cycloalkyl, an optionally substituted partially unsaturated or aromatic carbocyclic group, or an optionally substituted saturated, partially unsaturated, or aromatic heterocyclic group, wherein at least one of A₁ and A₂ is an optionally substituted aromatic carbocyclic group or an optionally substituted aromatic heterocyclic group;

X and W are independently O, S, NR, or absent, wherein R is hydrogen, optionally substituted (C₁-C₆)alkyl, or optionally substituted aryl(C₀-C₄)alkyl;

V is (C₁-C₆)alkyl, (C₂-C₆)alkenyl, (C₃-C₇)cycloalkyl, or absent, wherein when V is absent, W is absent; and

Y is (C₁-C₆) alkyl, (C₁-C₆)alkyl substituted with (C₃-C₇)cycloalkyl, (C₂-C₆) alkenyl, (C₃-C₇)cycloalkyl, or absent; and

Z is carbonyl, thiocarbonyl, imino, or C₁-C₆ alkylimino; and

R₁ and R₂ are independently hydrogen or methyl;

and with the further proviso that said compound of Formula (III) is not a compound of the formula (III-b):

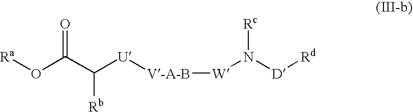

wherein
R^{a} is hydrogen, a substituted or unsubstituted alkyl or cycloalkyl group, a substituted or unsubstituted aryl group or a saturated or unsaturated, optionally substituted heterocyclic group;
R^{b} is hydrogen, a substituted or unsubstituted alkyl or cycloalkyl group, a substituted or unsubstituted aryl group, a saturated or unsaturated, optionally substituted heterocyclic group, an optionally substituted alkenyl group, an optionally substituted alkinyl group, —NR^{2′}SO₂R^{2″}, —NR^{2′}COOR^{2′}, —NR^{2′}COR^{2′}, —NR^{2′}CON(R^{2′})₂, or —N R^{2′}CSN(R^{2′})₂;
R^{2′} is hydrogen, a substituted or unsubstituted alkyl or cycloalkyl group, a substituted or unsubstituted aryl group or a saturated or unsaturated, optionally substituted heterocyclic group;
R^{2″} is a substituted or unsubstituted alkyl, alkenyl or cycloalkyl group, a substituted or unsubstituted aryl group, or a saturated or unsaturated, optionally substituted heterocyclic group;
U′ is a direct bond or a substituted or unsubstituted alkylene group;
V′ is a substituted or unsubstituted alkylene group, —NR^{2′}CO—, or —NR^{2′}SO₂—;
A and B are each independently an optionally substituted 1,3- or 1,4-bridging phenylene group or an optionally substituted 2,4- or 2,5-bridging thienylene group;
W′ is a direct bond or an optionally substituted alkylene group;
D′ is a direct bond or

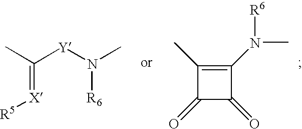

R^{c} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{d}, Y′, R⁵, or R⁶, if present, with formation of an optionally substituted heterocyclic ring system which includes the nitrogen atom to which R^{c} is bonded, and may be saturated or unsaturated and may contain further heteroatoms;
R^{d} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{c}, Y, R⁵ or R⁶, if present, with formation of an optionally substituted heterocyclic ring system which includes the nitrogen atom to which R^{d} is bonded and may be saturated or unsaturated and may contain further heteroatoms;

### X′ is CHNO₂, CHCN, O, N or S;

Y′ is a direct bond or an optionally substituted alkylene or alkine group;
R⁵ is absent, or is hydrogen, a substituted or unsubstituted alkyl or cycloalkyl group, —NO₂, —CN, —COR^{5′}, —COOR^{5′}, or is connected to one of R^{c}, Y′, R^{d}, or R⁶, if present, with formation of an optionally substituted carbocyclic or heterocyclic ring system which includes X′ and can be saturated or unsaturated and may optionally contain one or more additional heteroatoms;
R^{5′} is hydrogen, a substituted or unsubstituted alkyl or cycloalkyl group, a substituted or unsubstituted aryl group or a saturated or unsaturated, optionally substituted heterocyclic group which may optionally contain one or more additional heteroatoms;
R⁶ is a arylcarbonyl group, or a heteroarylcarbonyl group;
and wherein if A is a phenylene group and V′ is —NR^{2′}CO— or —NR^{2′} SO₂—, D′ is not a direct bond and X′ is not N;
and with the further proviso that said compound of Formula (III) is not a compound of the formula (III-c)

X^{1″}—Y^{1″}-Z^{1″}-W^{1″} (III-c)

wherein
X^{1″} is optionally substituted aryl, optionally substituted (C₁-C₈)alkyl-aryl, optionally substituted heteroaryl, or optionally substituted heteroarylalkyl;
Y^{1″} is —NR^{A}CSNR^{A}CO—, wherein R^{A} is independently hydrogen or lower alkyl;
Z^{1″} is optionally substituted phenylene, optionally substituted monocyclic heteroarylene, optionally substituted monocyclic non-aromatic heterocycle-diyl, or optionally substituted monocyclic cycloalkane-diyl;
W^{1″} is a group represented by the formula:

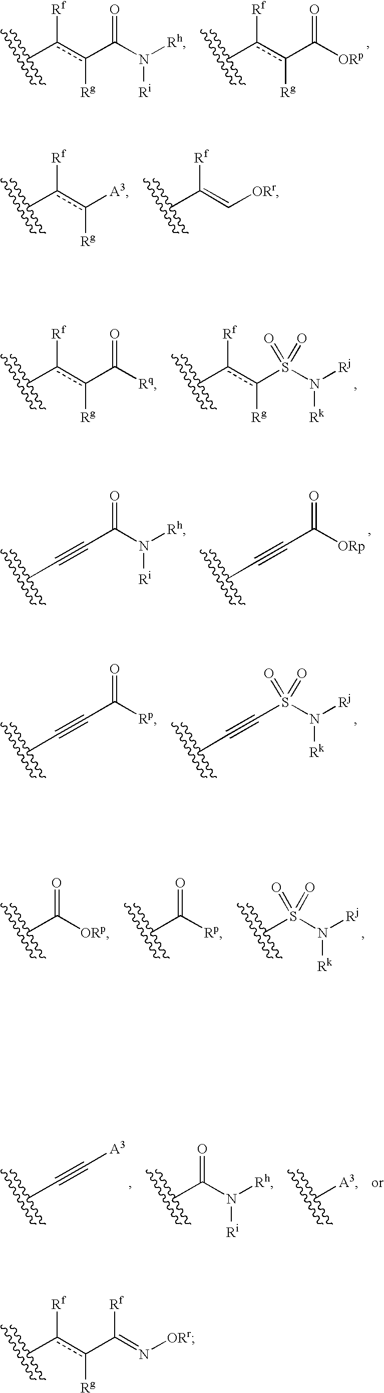

wherein
R^{f}, R^{g}, R^{h}, Rⁱ, R^{j}, and R^{k} are each independently hydrogen, halogen, optionally substituted lower alkyl, optionally substituted lower alkyloxy, optionally substituted lower alkylthio, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, optionally substituted non-aromatic heterocyclic group, or optionally substituted amino;
R^{p}, R^{q}, and R^{r} are each independently hydrogen, optionally substituted lower alkyl, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, or optionally substituted non-aromatic heterocyclic group;
A³ is an optionally substituted aryl or an optionally substituted heteroaryl group;
and with the further proviso that in said compound of Formula (III), Z¹ is not a 3-pyridyl group or a substituted 3-pyridyl group when Z² is —OH, —NH₂, —(C₁-C₆)alkylamino, di(C₁-C₆)alkylamino, an alkyl group, a substituted alkyl group, —(C₃-C₈) cycloalkyl group, a substituted —(C₃-C₈) cycloalkyl group, —(C₃-C₈) cycloalkyl-(C₁-C₆)alkyl group, —O-alkyl group, a substituted —O-alkyl group, —O—(C₃-C₈) cycloalkyl group, a substituted —O—(C₃-C₈) cycloalkyl group, a phenyl group, a fused-phenyl group, a phenyl group substituted with one or more independently selected halogen, —OCH₃, nitro, or dimethyl amino groups, a substituted fused-phenyl group substituted with one or more independently selected halogen, —OCH₃, nitro, or dimethyl amino groups, a —(C₁-C₈) alkyl-phenyl group, —O—(C₀-C₈) alkyl-phenyl group, —(C₃-C₆) alkylene group, —O—(C₃-C₆) alkylene group, —(C₃-C₆) alkynyl group, —O—(C₃-C₆) alkynyl group, —CO(C₁-C₆) alkyl group, —NHCO(C₁-C₆) alkyl group, —NHSO₂(C₁-C₆) alkyl group, —SO₂(C₁-C₆) alkyl group, or —SO₂(C₀-C₆)alkyl-phenyl group;
and with the further proviso that in said compound of Formula (III), Z¹ is not a furyl group or a substituted furyl group when D² and Z² are independently selected from the group consisting of hydrogen and methyl;
and with the further proviso that in said compound comprising structure (III), when D² is —CH₃ and Z² is an optionally substituted phenyl, Z¹ is not selected from the group consisting of —R³³, OR³⁴, and —NR³⁵R³⁶,
wherein
R³³ is hydrogen, optionally substituted (C₃-C₈)cycloalkyl, optionally substituted phenyl, optionally substituted heteroaryl or phenyl-(C₁-C₄) alkyl which is optionally substituted on the phenyl ring, (C₁-C₆)alkyl, (C₂-C₆)alkenyl, (C₂-C₆)alkynyl, the three latter radicals independently being optionally substituted with one or more radicals selected from the group consisting of halogen, (C₁-C₄)alkoxy, (C₁-C₄)alkylthio and NR³⁷R³⁸;
R³⁴ is (C₁-C₆)alkyl, (C₂-C₆)alkenyl, (C₂-C₆)alkynyl, the three latter radicals independently optionally substituted with one or more radicals selected from the group consisting of halogen, (C₁-C₄)alkoxy, (C₁-C₄)alkylthio and NR³⁷R³⁸, or R³⁴ is (C₃-C₆)cycloalkyl optionally substituted with one or more groups selected from the group consisting of halogen, (C₁-C₄)alkyl and (C₁-C₄)alkoxy, and (C₃-C₆)-cycloalkyl-(C₁-C₃)alkyl;
R³⁵ and R³⁶ are hydrogen, (C₁-C₆)alkyl, (C₂-C₆)alkenyl, (C₂-C₆)alkynyl, the 3 latter groups independently optionally substituted by one or more halogen radicals;
R³⁷ is independently selected from the group consisting of hydrogen, (C₁-C₄)alkyl, ((C₁-C₄)alkyl)carbonyl, ((C₁-C₄)alkoxy)carbonyl and CHO;
R³⁸ is independently selected from the group consisting of H and (C₁-C₄)alkyl;
and with the further proviso that in said compound of Formula (III), Z¹ and Z² are not both selected from the group consisting of hydrogen and alkyl;
and with the further proviso that in said compound of Formula (III), Z² is not hydrogen when D¹ and D² are both hydrogen;
and with the further proviso that in said compound of Formula (III), Z¹ is not (C₁-C₆) alkyl, (C₁-C₆) alkoxy, pyridyl, or aryl when D¹ and D² are both hydrogen and when Z is selected from the group consisting of hydrogen, a (C₁-C₆) alkyl group optionally substituted with 1 to 3 substituents independently selected from the group consisting of hydroxyl, (C₁-C₆) alkoxyl, carboxyl, (C₁-C₆) alkoxycarbonyl, lower alkylthio, fluorine, chlorine, bromine, iodine, amino, mono- or di-substituted amino, phthalimido and nitrogen-containing heterocyclic groups, a (C₃-C₈) cycloalkyl group, a (C₂-C₆) alkenyl group, an arylalkyl group optionally substituted with from 1 to 5 substituents independently selected from the group consisting of (C₁-C₆) alkyl, hydroxyl, (C₁-C₆) alkoxy, halogen, nitro, and amino groups, an aryl group optionally substituted with from 1 to 5 independently selected substituents selected from the group consisting of (C₁-C₆) alkyl, hydroxyl, (C₁-C₆) alkoxy, halogen, nitro, and amino groups, a substituted or unsubstituted heterocyclic group, a (C₁-C₆) alkanoyl group, a benzoyl or a naphthoyl group either of which is optionally substituted with from 1 to 5 independently selected substituents selected from the group consisting of (C₁-C₆) alkyl, hydroxyl, (C₁-C₆) alkoxy, halogen, nitro, and amino groups, a pyridylcarbonyl group, a (C₁-C₆) alkoxycarbonyl group or an amino group optionally substituted with (C₁-C₆) alkyl, arylalkyl having 7 to 15 carbon atoms, substituted or unsubstituted aryl, (C₁-C₆) alkanoyl, optionally substituted aroyl, and (C₁-C₆) alkylidene groups;
and with the further proviso that in said compound of Formula (III), Z¹ is not —O—(CH₂)ₜ—CH₃, wherein t is 0-4 and Z² is halophenyl;
and with the further proviso that in said compound of Formula (III), Z¹ is not a substituted pyridyl, pyrimidinyl, pyrazinyl, pyridazinyl group substituted with —N(R²¹)-(optionally substituted phenyl), wherein said R²¹ is hydrogen, aryl, formyl, (C₁-C₆)alkylcarbonyl, (C₁-C₆)alkyl, (C₁-C₆)alkyloxycarbonyl, (C₁-C₆)alkyl substituted with formyl, (C₁-C₆)alkylcarbonyl, (C₁-C₆)alkyloxycarbonyl, (C₁-C₆)alkylcarbonyloxy, or (C₁-C₆)alkyloxy(C₁-C₆)alkylcarbonyl optionally substituted with (C₁-C₆)alkyloxycarbonyl;
and with the further proviso that in said compound of Formula (II), Z¹ is not a 3-(4-trifluoromethyl-pyridinyl) group or a 3-(4-trifluoromethyl-pyridinyl N-oxide) group;
and with the further proviso that in said compound of Formula (III), Z¹ is not a substituted or unsubstituted group selected from the group consisting of

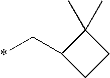

and a —(CH₂)ₜ-adamantanyl group, wherein t is 0-4;
and with the further proviso that in said compound of Formula (III), Z² is not a

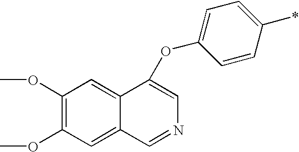

group, which is unsubstituted, or which is substituted with one or more substituents independently selected from the group consisting of halogens and alkyl groups;
and with the further proviso that in said compound of Formula (III), Z² is not a substituted or unsubstituted group selected from the group consisting of:

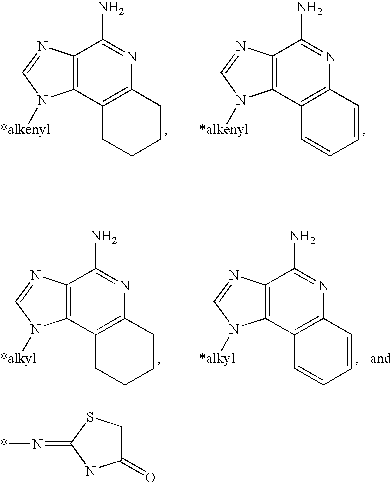

and with the further proviso that said compound of Formula (III), is not a compound set forth in Appendix A, Appendix B, or Appendix C.
147. A method of screening a test compound for replicase complex defect inducer activity comprising:

providing a test compound;

contacting the test compound with an isolated HCV replicase complex; and

identifying the test compound as an inducer of an HCV replicase complex defect when the level of p14 protein is increased.

148. The method according to claim 147, wherein the HCV replicon is a Con-1 replicon.
149. The method according to claim 147, where the HCV replicon has a mutation in an NS3 protein or fragment thereof.
150. The method according to claim 147, wherein the HCV replicon has a mutation at or within about 15 angstroms of C16 in an NS3 protein or fragment thereof.
151. The method according to claim 147, wherein the HCV replicon has an A39V mutation in an NS3 protein or fragment thereof
152. The method according to claim 147, wherein the HCV replicon has an C16S mutation in an NS3 protein or fragment thereof.
153. The method according to claim 147, wherein the level of NS3 production is reduced.
154. An inducer of a replicase complex defect identified by the method of claim 147, wherein said inducer is a compound of Formula (III)

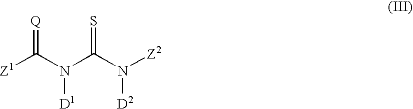

wherein
Q is oxygen or sulfur;
D¹ and D² are independently selected from the group consisting of hydrogen and methyl;
with the proviso that said compound of Formula (III) is not a compound of the formula (III-a):

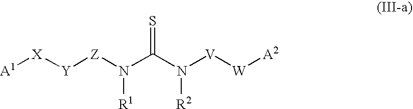

wherein

A₁ and A₂ are independently optionally substituted C₁-C₁₂ alkyl, optionally substituted mono- or di-(C₁-C₈ alkyl)amino, optionally substituted C₂-C₁₂ alkenyl, optionally substituted C₃-C₈ cycloalkyl, an optionally substituted partially unsaturated or aromatic carbocyclic group, or an optionally substituted saturated, partially unsaturated, or aromatic heterocyclic group, wherein at least one of A₁ and A₂ is an optionally substituted aromatic carbocyclic group or an optionally substituted aromatic heterocyclic group;

X and W are independently O, S, NR, or absent, wherein R is hydrogen, optionally substituted (C₁-C₆)alkyl, or optionally substituted aryl(C₀-C₄)alkyl;

V is (C₁-C₆)alkyl, (C₂-C₆)alkenyl, (C₃-C₇)cycloalkyl, or absent, wherein when V is absent, W is absent; and

Y is (C₁-C₆) alkyl, (C₁-C₆)alkyl substituted with (C₃-C₇)cycloalkyl, (C₂-C₆) alkenyl, (C₃-C₇)cycloalkyl, or absent; and

Z is carbonyl, thiocarbonyl, imino, or C₁-C₆ alkylimino; and

R₁ and R₂ are independently hydrogen or methyl;

and with the further proviso that said compound of Formula (III) is not a compound of the formula (III-b):

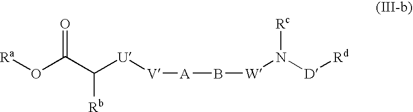

wherein
R^{a} is hydrogen, a substituted or unsubstituted alkyl or cycloalkyl group, a substituted or unsubstituted aryl group or a saturated or unsaturated, optionally substituted heterocyclic group;
R^{b} is hydrogen, a substituted or unsubstituted alkyl or cycloalkyl group, a substituted or unsubstituted aryl group, a saturated or unsaturated, optionally substituted heterocyclic group, an optionally substituted alkenyl group, an optionally substituted alkinyl group, —NR^{2′}SO₂R^{2″}, —NR^{2′}COOR^{2′}, —NR^{2′}COR^{2′}, —NR^{2′}CON(R^{2′})₂, or —N R^{2′}CSN(R^{2′})₂;
R^{2′} is hydrogen, a substituted or unsubstituted alkyl or cycloalkyl group, a substituted or unsubstituted aryl group or a saturated or unsaturated, optionally substituted heterocyclic group;
R^{2′} is a substituted or unsubstituted alkyl, alkenyl or cycloalkyl group, a substituted or unsubstituted aryl group, or a saturated or unsaturated, optionally substituted heterocyclic group;
U′ is a direct bond or a substituted or unsubstituted alkylene group;
V′ is a substituted or unsubstituted alkylene group, —NR^{2′}CO—, or —NR^{2′}SO₂—;
A and B are each independently an optionally substituted 1,3- or 1,4-bridging phenylene group or an optionally substituted 2,4- or 2,5-bridging thienylene group;
W′ is a direct bond or an optionally substituted alkylene group;
D′ is a direct bond or

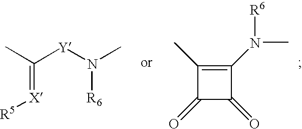

R^{c} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{d}, Y′, R⁵, or R⁶, if present, with formation of an optionally substituted heterocyclic ring system which includes the nitrogen atom to which R^{c} is bonded, and may be saturated or unsaturated and may contain further heteroatoms;
R^{d} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{c}, Y, R⁵ or R⁶, if present, with formation of an optionally substituted heterocyclic ring system which includes the nitrogen atom to which R^{d} is bonded and may be saturated or unsaturated and may contain further heteroatoms;

### X′ is CHNO₂, CHCN, O, N or S;

Y′ is a direct bond or an optionally substituted alkylene or alkine group;
R⁵ is absent, or is hydrogen, a substituted or unsubstituted alkyl or cycloalkyl group, —NO₂, —CN, —COR^{5′}, —COOR^{5′}, or is connected to one of R^{c}, Y′, R^{d}, or R⁶, if present, with formation of an optionally substituted carbocyclic or heterocyclic ring system which includes X′ and can be saturated or unsaturated and may optionally contain one or more additional heteroatoms;
R^{5′} is hydrogen, a substituted or unsubstituted alkyl or cycloalkyl group, a substituted or unsubstituted aryl group or a saturated or unsaturated, optionally substituted heterocyclic group which may optionally contain one or more additional heteroatoms;
R⁶ is a arylcarbonyl group, or a heteroarylcarbonyl group;
and wherein if A is a phenylene group and V′ is —NR^{2′}CO— or —NR^{2′} SO₂—, D′ is not a direct bond and X′ is not N;
and with the further proviso that said compound of Formula (III) is not a compound of the formula (III-c)

X^{1″}—Y^{1″}-Z^{1″}-W^{1″} (III-c)

wherein
X^{1″} is optionally substituted aryl, optionally substituted (C₁-C₈)alkyl-aryl, optionally substituted heteroaryl, or optionally substituted heteroarylalkyl;
Y^{1″} is —NR^{A}CSNR^{A}CO—, wherein R^{A} is independently hydrogen or lower alkyl;
Z^{1″} is optionally substituted phenylene, optionally substituted monocyclic heteroarylene, optionally substituted monocyclic non-aromatic heterocycle-diyl, or optionally substituted monocyclic cycloalkane-diyl;
W^{1″} is a group represented by the formula:

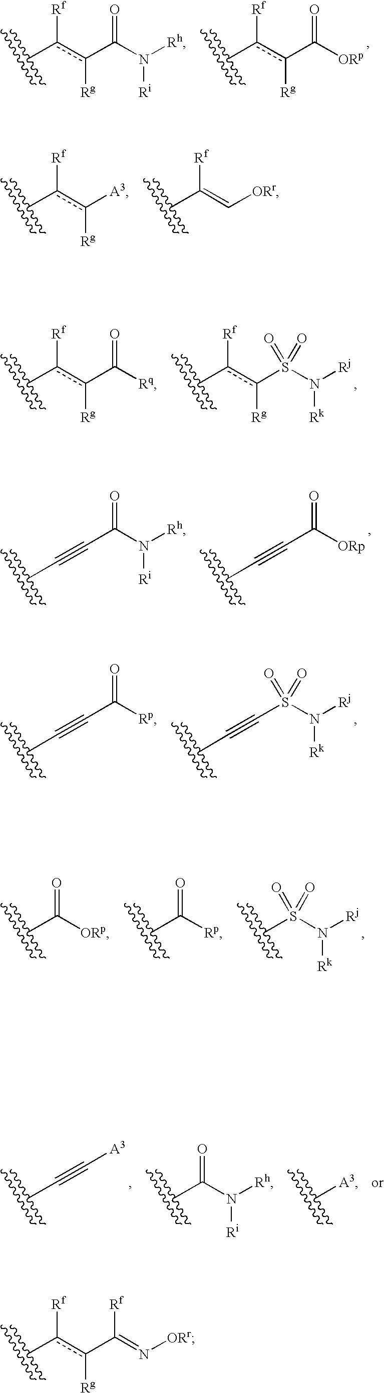

wherein
R^{f}, R^{g}, R^{h}, Rⁱ, R^{j}, and R^{k} are each independently hydrogen, halogen, optionally substituted lower alkyl, optionally substituted lower alkyloxy, optionally substituted lower alkylthio, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, optionally substituted non-aromatic heterocyclic group, or optionally substituted amino;
R^{p}, R^{q}, and R^{r} are each independently hydrogen, optionally substituted lower alkyl, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, or optionally substituted non-aromatic heterocyclic group;
A³ is an optionally substituted aryl or an optionally substituted heteroaryl group;
and with the further proviso that in said compound of Formula (III), Z¹ is not a 3-pyridyl group or a substituted 3-pyridyl group when Z² is —OH, —NH₂, —(C₁-C₆)alkylamino, di(C₁-C₆)alkylamino, an alkyl group, a substituted alkyl group, —(C₃-C₈) cycloalkyl group, a substituted —(C₃-C₈) cycloalkyl group, —(C₃-C₈) cycloalkyl-(C₁-C₆)alkyl group, —O-alkyl group, a substituted —O-alkyl group, —O—(C₃-C₈) cycloalkyl group, a substituted —O—(C₃-C₈) cycloalkyl group, a phenyl group, a fused-phenyl group, a phenyl group substituted with one or more independently selected halogen, —OCH₃, nitro, or dimethyl amino groups, a substituted fused-phenyl group substituted with one or more independently selected halogen, —OCH₃, nitro, or dimethyl amino groups, a —(C₁-C₈) alkyl-phenyl group, —O—(C₀-C₈) alkyl-phenyl group, —(C₃-C₆) alkylene group, —O—(C₃-C₆) alkylene group, —(C₃-C₆) alkynyl group, —O—(C₃-C₆) alkynyl group, —CO(C₁-C₆) alkyl group, —NHCO(C₁-C₆) alkyl group, —NHSO₂(C₁-C₆) alkyl group, —SO₂(C₁-C₆) alkyl group, or —SO₂(C₀-C₆)alkyl-phenyl group;
and with the further proviso that in said compound of Formula (III), Z¹ is not a furyl group or a substituted furyl group when D² and Z² are independently selected from the group consisting of hydrogen and methyl;
and with the further proviso that in said compound comprising structure (III), when D² is —CH₃ and Z² is an optionally substituted phenyl, Z¹ is not selected from the group consisting of —R³³, OR³⁴, and —NR³⁵R³⁶,
wherein
R³³ is hydrogen, optionally substituted (C₃-C₈)cycloalkyl, optionally substituted phenyl, optionally substituted heteroaryl or phenyl-(C₁-C₄) alkyl which is optionally substituted on the phenyl ring, (C₁-C₆)alkyl, (C₂-C₆)alkenyl, (C₂-C₆)alkynyl, the three latter radicals independently being optionally substituted with one or more radicals selected from the group consisting of halogen, (C₁-C₄)alkoxy, (C₁-C₄)alkylthio and NR³⁷R³⁸;
R³⁴ is (C₁-C₆)alkyl, (C₂-C₆)alkenyl, (C₂-C₆)alkynyl, the three latter radicals independently optionally substituted with one or more radicals selected from the group consisting of halogen, (C₁-C₄)alkoxy, (C₁-C₄)alkylthio and NR³⁷R³⁸, or R³⁴ is (C₃-C₆)cycloalkyl optionally substituted with one or more groups selected from the group consisting of halogen, (C₁-C₄)alkyl and (C₁-C₄)alkoxy, and (C₃-C₆)-cycloalkyl-(C₁-C₃)alkyl;
R³⁵ and R³⁶ are hydrogen, (C₁-C₆)alkyl, (C₂-C₆)alkenyl, (C₂-C₆)alkynyl, the 3 latter groups independently optionally substituted by one or more halogen radicals;
R³⁷ is independently selected from the group consisting of hydrogen, (C₁-C₄)alkyl, ((C₁-C₄)alkyl)carbonyl, ((C₁-C₄)alkoxy)carbonyl and CHO;
R³⁸ is independently selected from the group consisting of H and (C₁-C₄)alkyl;
and with the further proviso that in said compound of Formula (III), Z¹ and Z² are not both selected from the group consisting of hydrogen and alkyl;
and with the further proviso that in said compound of Formula (III), Z² is not hydrogen when D¹ and D² are both hydrogen;
and with the further proviso that in said compound of Formula (III), Z¹ is not (C₁-C₆) alkyl, (C₁-C₆) alkoxy, pyridyl, or aryl when D¹ and D² are both hydrogen and when Z² is selected from the group consisting of hydrogen, a (C₁-C₆) alkyl group optionally substituted with 1 to 3 substituents independently selected from the group consisting of hydroxyl, (C₁-C₆) alkoxyl, carboxyl, (C₁-C₆) alkoxycarbonyl, lower alkylthio, fluorine, chlorine, bromine, iodine, amino, mono- or di-substituted amino, phthalimido and nitrogen-containing heterocyclic groups, a (C₃-C₈) cycloalkyl group, a (C₂-C₆) alkenyl group, an arylalkyl group optionally substituted with from 1 to 5 substituents independently selected from the group consisting of (C₁-C₆) alkyl, hydroxyl, (C₁-C₆) alkoxy, halogen, nitro, and amino groups, an aryl group optionally substituted with from 1 to 5 independently selected substituents selected from the group consisting of (C₁-C₆) alkyl, hydroxyl, (C₁-C₆) alkoxy, halogen, nitro, and amino groups, a substituted or unsubstituted heterocyclic group, a (C₁-C₆) alkanoyl group, a benzoyl or a naphthoyl group either of which is optionally substituted with from 1 to 5 independently selected substituents selected from the group consisting of (C₁-C₆) alkyl, hydroxyl, (C₁-C₆) alkoxy, halogen, nitro, and amino groups, a pyridylcarbonyl group, a (C₁-C₆) alkoxycarbonyl group or an amino group optionally substituted with (C₁-C₆) alkyl, arylalkyl having 7 to 15 carbon atoms, substituted or unsubstituted aryl, (C₁-C₆) alkanoyl, optionally substituted aroyl, and (C₁-C₆) alkylidene groups;
and with the further proviso that in said compound of Formula (III), Z¹ is not —O—(CH₂)ₜ—CH₃, wherein t is 0-4 and Z² is halophenyl;
and with the further proviso that in said compound of Formula (III), Z¹ is not a substituted pyridyl, pyrimidinyl, pyrazinyl, pyridazinyl group substituted with —N(R²¹)-(optionally substituted phenyl), wherein said R²¹ is hydrogen, aryl, formyl, (C₁-C₆)alkylcarbonyl, (C₁-C₆)alkyl, (C₁-C₆)alkyloxycarbonyl, (C₁-C₆)alkyl substituted with formyl, (C₁-C₆)alkylcarbonyl, (C₁-C₆)alkyloxycarbonyl, (C₁-C₆)alkylcarbonyloxy, or (C₁-C₆)alkyloxy(C₁-C₆)alkylcarbonyl optionally substituted with (C₁-C₆)alkyloxycarbonyl;
and with the further proviso that in said compound of Formula (III), Z² is not a 3-(4-trifluoromethyl-pyridinyl) group or a 3-(4-trifluoromethyl-pyridinyl N-oxide) group;
and with the further proviso that in said compound of Formula (III), Z¹ is not a substituted or unsubstituted group selected from the group consisting of

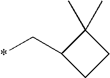

and a —(CH₂)ₜ-adamantanyl group, wherein t is 0-4;
and with the further proviso that in said compound of Formula (III), Z² is not a

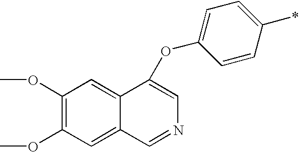

group, which is unsubstituted, or which is substituted with one or more substituents independently selected from the group consisting of halogens and alkyl groups;
and with the further proviso that in said compound of Formula (III), Z² is not a substituted or unsubstituted group selected from the group consisting of:

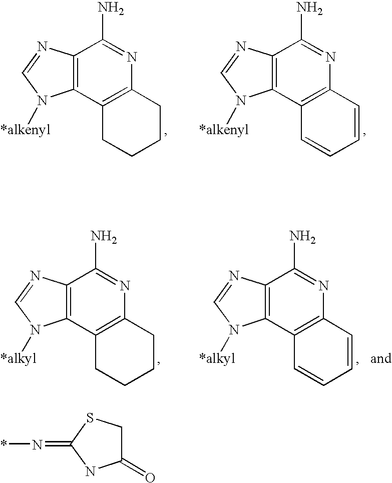

and with the further proviso that said compound of Formula (III), is not a compound set forth in Appendix A, Appendix B, or Appendix C.
155. A method of screening a test compound for replicase complex defect inducer activity comprising:

providing a test compound;

contacting the test compound with an isolated HCV polyprotein or fragment thereof; and

identifying the test compound as an inducer of an HCV replicase complex defect when the level of p14 protein is increased.

156. The method according to claim 155, wherein the HCV replicon is a Con-1 replicon.
157. The method according to claim 155, where the HCV replicon has a mutation in an NS3 protein or fragment thereof.
158. The method according to claim 155, wherein the HCV replicon has a mutation at or within about 15 angstroms of C16 in an NS3 protein or fragment thereof.
159. The method according to claim 155, wherein the HCV replicon has an A39V mutation in an NS3 protein or fragment thereof.
160. The method according to claim 155, wherein the HCV replicon has an C16S mutation in an NS3 protein or fragment thereof.
161. The method according to claim 155, wherein the level of NS3 production is reduced.
162. An inducer of a replicase complex defect identified by the method of claim 155, wherein said inducer is a compound of Formula (III)

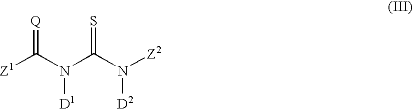

wherein
Q is oxygen or sulfur;
D¹ and D² are independently selected from the group consisting of hydrogen and methyl;
with the proviso that said compound of Formula (III) is not a compound of the formula (III-a):

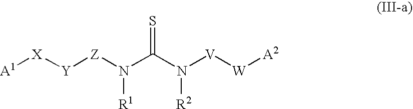

wherein

A₁ and A₂ are independently optionally substituted C₁-C₁₂ alkyl, optionally substituted mono- or di-(C₁-C₈ alkyl)amino, optionally substituted C₂-C₁₂ alkenyl, optionally substituted C₃-C₈ cycloalkyl, an optionally substituted partially unsaturated or aromatic carbocyclic group, or an optionally substituted saturated, partially unsaturated, or aromatic heterocyclic group, wherein at least one of A₁ and A₂ is an optionally substituted aromatic carbocyclic group or an optionally substituted aromatic heterocyclic group;

X and W are independently O, S, NR, or absent, wherein R is hydrogen, optionally substituted (C₁-C₆)alkyl, or optionally substituted aryl(C₀-C₄)alkyl;

V is (C₁-C₆)alkyl, (C₂-C₆)alkenyl, (C₃-C₇)cycloalkyl, or absent, wherein when V is absent, W is absent; and

Y is (C₁-C₆) alkyl, (C₁-C₆)alkyl substituted with (C₃-C₇)cycloalkyl, (C₂-C₆) alkenyl, (C₃-C₇)cycloalkyl, or absent; and

Z is carbonyl, thiocarbonyl, imino, or C₁-C₆ alkylimino; and

R₁ and R₂ are independently hydrogen or methyl;

and with the further proviso that said compound of Formula (III) is not a compound of the formula (III-b):

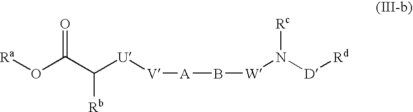

wherein
R^{a} is hydrogen, a substituted or unsubstituted alkyl or cycloalkyl group, a substituted or unsubstituted aryl group or a saturated or unsaturated, optionally substituted heterocyclic group;
R^{b} is hydrogen, a substituted or unsubstituted alkyl or cycloalkyl group, a substituted or unsubstituted aryl group, a saturated or unsaturated, optionally substituted heterocyclic group, an optionally substituted alkenyl group, an optionally substituted alkinyl group, —NR^{2′}SO₂R^{2″}, —NR^{2′}COOR^{2′}, —NR^{2′}COR^{2′}, —NR^{2′}CON(R^{2′})₂, or —N R^{2′}CSN(R^{2′})₂;
R^{2′} is hydrogen, a substituted or unsubstituted alkyl or cycloalkyl group, a substituted or unsubstituted aryl group or a saturated or unsaturated, optionally substituted heterocyclic group;
R^{2″} is a substituted or unsubstituted alkyl, alkenyl or cycloalkyl group, a substituted or unsubstituted aryl group, or a saturated or unsaturated, optionally substituted heterocyclic group;
U′ is a direct bond or a substituted or unsubstituted alkylene group;
V′ is a substituted or unsubstituted alkylene group, —NR^{2′}CO—, or —NR^{2′}SO₂—;
A and B are each independently an optionally substituted 1,3- or 1,4-bridging phenylene group or an optionally substituted 2,4- or 2,5-bridging thienylene group;
W′ is a direct bond or an optionally substituted alkylene group;
D′ is a direct bond or

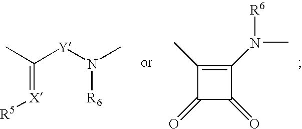

R^{c} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{d}, Y′, R⁵, or R⁶, if present, with formation of an optionally substituted heterocyclic ring system which includes the nitrogen atom to which R^{c} is bonded, and may be saturated or unsaturated and may contain further heteroatoms;
R^{d} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{c}, Y, R⁵ or R⁶, if present, with formation of an optionally substituted heterocyclic ring system which includes the nitrogen atom to which R^{d} is bonded and may be saturated or unsaturated and may contain further heteroatoms;

### X′ is CHNO₂, CHCN, O, N or S;

Y′ is a direct bond or an optionally substituted alkylene or alkine group;
R⁵ is absent, or is hydrogen, a substituted or unsubstituted alkyl or cycloalkyl group, —NO₂, —CN, —COR^{5′}, —COOR^{5′}, or is connected to one of R^{c}, Y′, R^{d}, or R⁶, if present, with formation of an optionally substituted carbocyclic or heterocyclic ring system which includes X′ and can be saturated or unsaturated and may optionally contain one or more additional heteroatoms;
R^{5′} is hydrogen, a substituted or unsubstituted alkyl or cycloalkyl group, a substituted or unsubstituted aryl group or a saturated or unsaturated, optionally substituted heterocyclic group which may optionally contain one or more additional heteroatoms;
R⁶ is a arylcarbonyl group, or a heteroarylcarbonyl group;
and wherein if A is a phenylene group and V′ is —NR^{2′}CO— or —NR^{2′} SO₂—, D′ is not a direct bond and X′ is not N;
and with the further proviso that said compound of Formula (III) is not a compound of the formula (III-c)

X^{1″}—Y^{1″}-Z^{1″}-W^{1″} (III-c)

wherein
X^{1″} is optionally substituted aryl, optionally substituted (C₁-C₈)alkyl-aryl, optionally substituted heteroaryl, or optionally substituted heteroarylalkyl;
Y^{1″} is —NR^{A}CSNR^{A}CO—, wherein R^{A} is independently hydrogen or lower alkyl;
Z^{1″} is optionally substituted phenylene, optionally substituted monocyclic heteroarylene, optionally substituted monocyclic non-aromatic heterocycle-diyl, or optionally substituted monocyclic cycloalkane-diyl;
W^{1″} is a group represented by the formula:

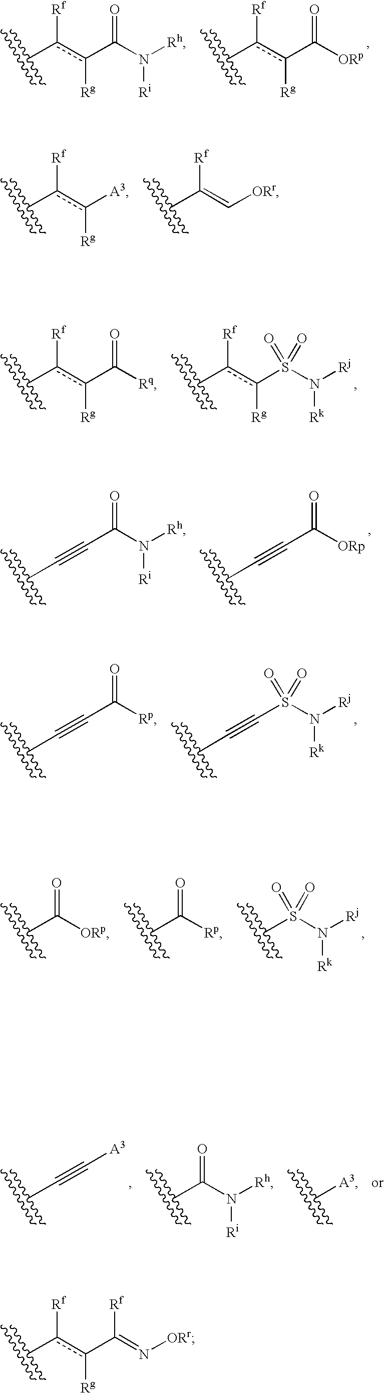

wherein
R^{f}, R^{g}, R^{h}, Rⁱ, R^{j}, and R^{k} are each independently hydrogen, halogen, optionally substituted lower alkyl, optionally substituted lower alkyloxy, optionally substituted lower alkylthio, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, optionally substituted non-aromatic heterocyclic group, or optionally substituted amino;
R^{p}, R^{q}, and R^{r} are each independently hydrogen, optionally substituted lower alkyl, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, or optionally substituted non-aromatic heterocyclic group;
A³ is an optionally substituted aryl or an optionally substituted heteroaryl group;
and with the further proviso that in said compound of Formula (III), Z¹ is not a 3-pyridyl group or a substituted 3-pyridyl group when Z² is —OH, —NH₂, —(C₁-C₆)alkylamino, di(C₁-C₆)alkylamino, an alkyl group, a substituted alkyl group, —(C₃-C₈) cycloalkyl group, a substituted —(C₃-C₈) cycloalkyl group, —(C₃-C₈) cycloalkyl-(C₁-C₆)alkyl group, —O-alkyl group, a substituted —O-alkyl group, —O—(C₃-C₈) cycloalkyl group, a substituted —O—(C₃-C₈) cycloalkyl group, a phenyl group, a fused-phenyl group, a phenyl group substituted with one or more independently selected halogen, —OCH₃, nitro, or dimethyl amino groups, a substituted fused-phenyl group substituted with one or more independently selected halogen, —OCH₃, nitro, or dimethyl amino groups, a —(C₁-C₈) alkyl-phenyl group, —O—(C₀-C₈) alkyl-phenyl group, —(C₃-C₆) alkylene group, —O—(C₃-C₆) alkylene group, —(C₃-C₆) alkynyl group, —O—(C₃-C₆) alkynyl group, —CO(C₁-C₆) alkyl group, —NHCO(C₁-C₆) alkyl group, —NHSO₂(C₁-C₆) alkyl group, —SO₂(C₁-C₆) alkyl group, or —SO₂(C₀-C₆)alkyl-phenyl group;
and with the further proviso that in said compound of Formula (III), Z¹ is not a furyl group or a substituted furyl group when D² and Z² are independently selected from the group consisting of hydrogen and methyl;
and with the further proviso that in said compound comprising structure (III), when D² is —CH₃ and Z² is an optionally substituted phenyl, Z¹ is not selected from the group consisting of —R³³, OR³⁴, and —NR³⁵R³⁶,
wherein
R³³ is hydrogen, optionally substituted (C₃-C₈)cycloalkyl, optionally substituted phenyl, optionally substituted heteroaryl or phenyl-(C₁-C₄) alkyl which is optionally substituted on the phenyl ring, (C₁-C₆)alkyl, (C₂-C₆)alkenyl, (C₂-C₆)alkynyl, the three latter radicals independently being optionally substituted with one or more radicals selected from the group consisting of halogen, (C₁-C₄)alkoxy, (C₁-C₄)alkylthio and NR³⁷R³⁸;
R³⁴ is (C₁-C₆)alkyl, (C₂-C₆)alkenyl, (C₂-C₆)alkynyl, the three latter radicals independently optionally substituted with one or more radicals selected from the group consisting of halogen, (C₁-C₄)alkoxy, (C₁-C₄)alkylthio and NR³⁷R³⁸, or R³⁴ is (C₃-C₆)cycloalkyl optionally substituted with one or more groups selected from the group consisting of halogen, (C₁-C₄)alkyl and (C₁-C₄)alkoxy, and (C₃-C₆)-cycloalkyl-(C₁-C₃)alkyl;
R³⁵ and R³⁶ are hydrogen, (C₁-C₆)alkyl, (C₂-C₆)alkenyl, (C₂-C₆)alkynyl, the 3 latter groups independently optionally substituted by one or more halogen radicals;
R³⁷ is independently selected from the group consisting of hydrogen, (C₁-C₄)alkyl, ((C₁-C₄)alkyl)carbonyl, ((C₁-C₄)alkoxy)carbonyl and CHO;
R³⁸ is independently selected from the group consisting of H and (C₁-C₄)alkyl;
and with the further proviso that in said compound of Formula (III), Z¹ and Z² are not both selected from the group consisting of hydrogen and alkyl;
and with the further proviso that in said compound of Formula (III), Z² is not hydrogen when D¹ and D² are both hydrogen;
and with the further proviso that in said compound of Formula (III), Z¹ is not (C₁-C₆) alkyl, (C₁-C₆) alkoxy, pyridyl, or aryl when D¹ and D² are both hydrogen and when Z² is selected from the group consisting of hydrogen, a (C₁-C₆) alkyl group optionally substituted with 1 to 3 substituents independently selected from the group consisting of hydroxyl, (C₁-C₆) alkoxyl, carboxyl, (C₁-C₆) alkoxycarbonyl, lower alkylthio, fluorine, chlorine, bromine, iodine, amino, mono- or di-substituted amino, phthalimido and nitrogen-containing heterocyclic groups, a (C₃-C₈) cycloalkyl group, a (C₂-C₆) alkenyl group, an arylalkyl group optionally substituted with from 1 to 5 substituents independently selected from the group consisting of (C₁-C₆) alkyl, hydroxyl, (C₁-C₆) alkoxy, halogen, nitro, and amino groups, an aryl group optionally substituted with from 1 to 5 independently selected substituents selected from the group consisting of (C₁-C₆) alkyl, hydroxyl, (C₁-C₆) alkoxy, halogen, nitro, and amino groups, a substituted or unsubstituted heterocyclic group, a (C₁-C₆) alkanoyl group, a benzoyl or a naphthoyl group either of which is optionally substituted with from 1 to 5 independently selected substituents selected from the group consisting of (C₁-C₆) alkyl, hydroxyl, (C₁-C₆) alkoxy, halogen, nitro, and amino groups, a pyridylcarbonyl group, a (C₁-C₆) alkoxycarbonyl group or an amino group optionally substituted with (C₁-C₆) alkyl, arylalkyl having 7 to 15 carbon atoms, substituted or unsubstituted aryl, (C₁-C₆) alkanoyl, optionally substituted aroyl, and (C₁-C₆) alkylidene groups;
and with the further proviso that in said compound of Formula (III), Z¹ is not —O—(CH₂)ₜ—CH₃, wherein t is 0-4 and Z² is halophenyl;
and with the further proviso that in said compound of Formula (III), Z¹ is not a substituted pyridyl, pyrimidinyl, pyrazinyl, pyridazinyl group substituted with —N(R²¹)-(optionally substituted phenyl), wherein said R²¹ is hydrogen, aryl, formyl, (C₁-C₆)alkylcarbonyl, (C₁-C₆)alkyl, (C₁-C₆)alkyloxycarbonyl, (C₁-C₆)alkyl substituted with formyl, (C₁-C₆)alkylcarbonyl, (C₁-C₆)alkyloxycarbonyl, (C₁-C₆)alkylcarbonyloxy, or (C₁-C₆)alkyloxy(C₁-C₆)alkylcarbonyl optionally substituted with (C₁-C₆)alkyloxycarbonyl;
and with the further proviso that in said compound of Formula (III), Z¹ is not a 3-(4-trifluoromethyl-pyridinyl) group or a 3-(4-trifluoromethyl-pyridinyl N-oxide) group;
and with the further proviso that in said compound of Formula (III), Z¹ is not a substituted or unsubstituted group selected from the group consisting of

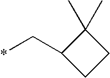

and a —(CH₂)ₜ-adamantanyl group, wherein t is 0-4;
and with the further proviso that in said compound of Formula (III), Z² is not a

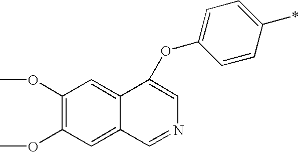

group, which is unsubstituted, or which is substituted with one or more substituents independently selected from the group consisting of halogens and alkyl groups;
and with the further proviso that in said compound of Formula (III), Z² is not a substituted or unsubstituted group selected from the group consisting of:

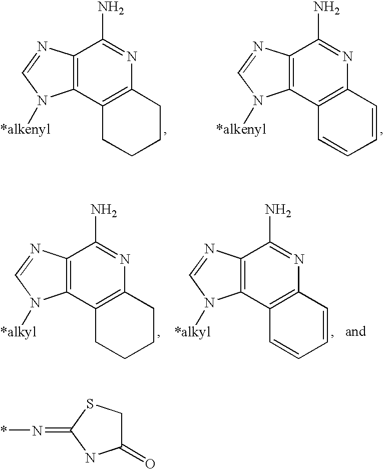

and with the further proviso that said compound of Formula (III), is not a compound set forth in Appendix A, Appendix B, or Appendix C.
163. An isolated replicase complex comprising a mutation in an NS3 at or within about 15 angstroms of C16.
164. An isolated replicase complex comprising an A39V mutation in an NS3 protein or fragment thereof.
165. An isolated replicase complex comprising a C16S mutation in an NS3 protein or fragment thereof.
166. An isolated polyprotein or fragment thereof that comprises an NS3 protein or fragment thereof having a mutation at or within about 15 angstroms of C16.
167. An isolated polyprotein or fragment thereof that comprises an NS3 protein or fragment thereof with an A39V mutation.
168. An isolated polyprotein or fragment thereof that comprises an NS3 protein or fragment thereof with a C16S mutation.

### DEFINITIONS

When referring to proteins and nucleic acids herein, “derived” refers to either directly (for example, by looking at the sequence of a known protein or nucleic acid and preparing a protein or nucleic acid having a sequence similar, at least in part, to the sequence of the known protein or nucleic acid) or indirectly (for example, by obtaining a protein or nucleic acid from an organism which is related to a known protein or nucleic acid) obtaining a protein or nucleic acid from a known protein or nucleic acid. Other methods of “deriving” a protein or nucleic acid from a known protein or nucleic acid are known to one of skill in the art.

“Heterologous” means not naturally occurring together. A mutant HCV protein can be heterologous in comparison to the rest of the HCV strain genotype.

A “mutant” refers to a cell expressing an HCV virion, HCV replicon, HCV replicase complex or HCV polypeptide or fragment thereof, where the HCV virion, HCV replicon, HCV replicase complex or HCV polypeptide or fragment thereof shows a higher resistance to a replicase complex defect inducer (RCDI) than a wild type HCV virion, HCV replicon, HCV replicase complex or HCV polypeptide or fragment thereof shows to the same RCDI. In an embodiment, resistance may be evidenced for example by greater growth of a mutant clone in the presence of an RCDI relative to the growth of a wild type clone in the presence of the same RCDI. In another embodiment, resistance may be evidenced by an increase in viral mRNA in a mutant in the presence of an RCDI as compared with a wild type in the presence of the same RCDI.

Susceptibility, or sensitivity, of a cell to an RCDI refers to the inability of the cell to grow in the presence of an RCDI. Determination of resistance or susceptibility of a cell to a test compound may be accomplished, for example, by determining EC₅₀, EC₉₀, or both of an RCDI.

Susceptibility, or sensitivity, of an HCV virion, HCV replicon, HCV replicase complex or HCV polypeptide or fragment thereof refers to the inability or reduced ability of the HCV virion, HCV replicon, HCV replicase complex or HCV polypeptide or fragment thereof to replicate in the presence of an RCDI. Determination of resistance or susceptibility of an HCV virion, HCV replicon, HCV replicase complex or HCV polypeptide or fragment thereof to an RCDI may be accomplished, for example, by comparing mRNA levels in an HCV virion, HCV replicon, HCV replicase complex or HCV polypeptide or fragment thereof before and after treatment with the RCDI. In another embodiment, determination of resistance or susceptibility of an HCV virion, HCV replicon, HCV replicase complex or HCV polypeptide or fragment thereof to an RCDI may be accomplished, for example, by comparing mRNA levels from an HCV virion, HCV replicon, HCV replicase complex or HCV polypeptide or fragment thereof after treatment with an RCDI with wild type mRNA levels of the HCV virion, HCV replicon, HCV replicase complex or HCV polypeptide or fragment thereof.

A “wild type clone” can be any untreated clone, such as a cell expressing an HCV virion, HCV replicon, HCV replicase complex, or HCV polyprotein or fragment thereof that has not been treated with a selective agent. Moreover, a wild type clone can contain any HCV virion, HCV replicon, HCV replicase complex, or HCV polyprotein or fragment thereof, as described herein, before selection.

As will be apparent to one of skill in the art, a virion as used herein includes a complete virus particle. For example, an HCV virion includes the RNA and protein coat of HCV. In an embodiment of the present invention, an HCV virion may be used as an alternative to an HCV replicon, HCV replicase complex or HCV polypeptide or fragment thereof in any of the methods of the present invention where use of an HCV replicon, replicase comples or polypeptide or fragment thereof has been described.

The present invention relates to replicase complex defect inducers (RCDIs) and their use in methods of screening and treatment. The present invention also provides a variety of compositions that are identified by the methods of the present invention. The present invention includes HCV virions, replicons, replicase complexes, and polyproteins that are useful in screening for replicase complex defect inducers. The present invention also provides methods of screening for replicase complex defect inducer compounds. In this manner, the present invention is useful for identifying replicase complex defect inducers. The present invention provides replicase complex defect inducer compounds identified by the methods of the present invention. The present invention further provides methods of treatment for hepatitis C virus and other diseases using replicase complex defect inducers. The present invention is useful for inhibition of hepatitis C virus replication and for prevention and treatment of hepatitis C viral infection.

### A. Replicase Complex Defect Inducers (RCDIs)

In some viruses, including positive strand RNA viruses, replication is performed by a multi-protein-nucleic acid complex referred to as a replicase complex. In an embodiment, a replicase complex is an active complex comprising polypeptide and nucleic acid molecules. A replicase complex is typically capable of producing complete and accurate viral replication. A replicase complex may comprise a single or double-stranded nucleic acid molecule. A replicase complex may also comprise a positive strand, a negative strand, or both positive and negative strands of a nucleic acid molecule. In a preferred embodiment, a replicase complex is an active complex comprising polypeptide and RNA molecules. In another preferred embodiment, a replicase complex is capable of producing complete and accurate viral replicon RNA synthesis under cell-free conditions suitable for viral RNA replication. In a highly preferred embodiment, a replicase complex is capable of producing full-length viral RNA.

A replicase complex may be isolated. An isolated replicase complex is a replicase complex that has been removed from its cellular environment, for example by being removed from a cell expressing a viral replicon RNA. The isolated replicase complex may be separated from the cell nucleus, chromosomal DNA, and cytoplasmic materials, for example. The membrane fraction of a cell expressing viral replicase RNA provides a non-limiting example of an isolated replicase complex. An isolated replicase complex may comprise one or more polypeptides expressed from a recombinant expression system, so long as the replicase complex remains capable of complete and accurate viral replicon RNA synthesis. For example, the replicase complex of HCV may include the NS5B protein, which has RNA-dependent RNA polymerase activity.

In one embodiment of the present invention, compounds that act as inducers of a replicase complex defect are described. In an embodiment of the present invention, a replicase complex defect inducer may cause any type or degree of incorrect assembly or any lack of assembly of a replicase complex. In another embodiment, a replicase complex defect inducer may cause any temporal effect on assembly of a replicase complex. In an embodiment, a replicase complex defect inducer may permit partial or complete assembly of a nonfunctional replicase complex. In an embodiment, a nonfunctional replicase complex cannot replicate viral RNA. In another embodiment, a nonfunctional replicase complex cannot replicate a complete viral RNA. In a further embodiment, a nonfunctional replicase complex cannot replicate an accurate viral RNA.

As used herein, a replicase complex defect inducer (RCDI) is any molecule that inhibits functional replicase complex assembly. In a preferred embodiment, an RCDI inhibits replicase complex assembly but does not inhibit the active site of hepatitis C virus NS3 protease. In another preferred embodiment, an RCDI inhibits replicase complex assembly but does not inhibit the active site of hepatitis C virus NS5B polymerase. In a highly preferred embodiment, an RCDI inhibits replicase complex assembly but does not inhibit the active site of hepatitis C virus NS3 protease or the active site of hepatitis C virus NS5B polymerase.

By way of non-limiting example, a replicase complex defect inducer may be a chemical, nucleic acid, polypeptide, amino acid, or any other compound that inhibits functional replicase complex assembly. The mechanism of action of the RCDIs is different from other classes of hepatitis C virus inhibitors that typically act by directly inhibiting the active site of an HCV protease or polymerase.

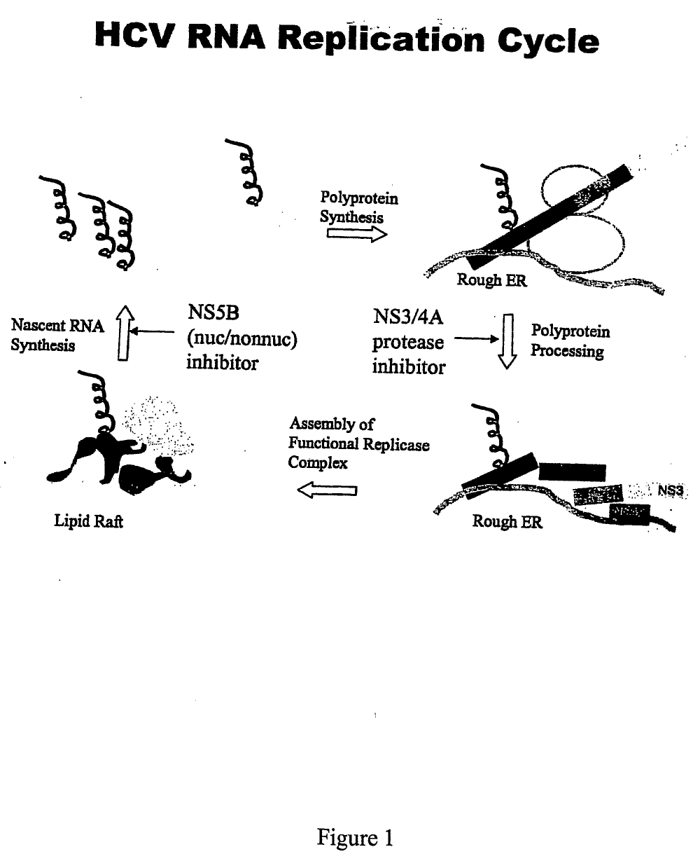

As illustrated in FIG. 1, in one embodiment and without intending to limit this or other embodiments to a particular mechanism, an RCDI may inhibit the formation of a functional replicase complex by causing changes in the viral protein composition of the replicase complex, such as miscleavage of the HCV polyprotein.

Miscleavage includes without limitation cleavage of an HCV polyprotein at a site other than or in addition to a site which is cleaved during HCV replication in an untreated cell. For example, miscleavage includes cleavage at sites other than the NS2-NS3, NS3-NS4A, NS4A-NS4B, NS4B-NS5A, and NS5A-NS5B cleavage sites. Miscleavage can occur between NS2-NS3, NS3-NS4A, NS4A-NS4B, NS4B-NS5A, or NS5A-NS5B. In an embodiment, the miscleavage may be cleavage at a site that is not generally cleaved or an increased or decreased level of cleavage at a site that is generally cleaved. Miscleavage may also occur at more than one location. For example, miscleavage may occur between NS2-NS3 and NS3-NS4A or between NS2-NS3, NS3-NS4A, and NS4A-NS4B. Alternatively, miscleavage may occur at any other combination of locations. In a preferred embodiment, a miscleavage occurs between NS3-NS4A.

In an embodiment, an RCDI can inhibit replicase complex assembly by interfering with the molecular interaction between viral proteins, for example between NS3 and NS4A. In a further embodiment, an RCDI can inhibit replicase complex assembly by interfering with the interaction between the viral nonstructural proteins and host factors.

RNA synthesis proceeds as a two-step process: initiation and elongation. In initiation, an initiated template RNA is formed in which only a portion of the newly synthesized positive or negative strand RNA is made using a minus or plus strand template. Upon initiation, the partial transcripts may be unable to dissociate from the RNA polymerase. In elongation, the remainder of the positive or negative strand RNA transcript is synthesized. In an embodiment, an RCDI of the present invention blocks replication prior to initiation. In another embodiment, an RCDI blocks replication after initiation. In an embodiment, an RCDI blocks replication prior to elongation. In another embodiment, an RCDI blocks replication after elongation. In an embodiment, an RCDI blocks replication prior to both initiation and elongation. In another embodiment, an RCDI blocks replication after both initiation and elongation.

In an embodiment of the present invention, an RCDI may inhibit replicase complex assembly by more than about 2%, more than about 5%, more than about 10%, more than about 20%, more than about 30%, more than about 40%, more than about 50%, more than about 60%, more than about 70%, more than about 80%, more than about 90%, more than about 95%, more than about 98%, or by more than about 99% as measured by the decrease in RNA replication in the presence of an RCDI compared with RNA replication in the absence of an RCDI.

Inhibition of HCV replication by a replicase complex defect inducer may be measured by any means available to the skilled artisan. For example, any technique for measuring EC₅₀ or EC₉₀ may be used. Techniques of spectrophotometry, gel electrophoresis, antibody hybridization, dot blot or any other technique may be used to assess inhibition of HCV replication.

In an embodiment, an RCDI is a substituted aryl acylthiourea or a metabolite thereof. In an embodiment, suitable aryl acylthioureas include compounds of Formula I:

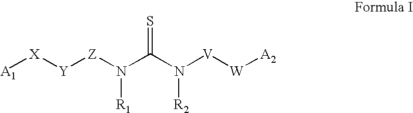

or a metabolite or a pharmaceutically acceptable salt thereof, wherein

A₁ and A₂ are independently optionally substituted C₁-C₁₂ alkyl, optionally substituted mono- or di-(C₁-C₈ alkyl)amino, optionally substituted C₂-C₁₂ alkenyl, optionally substituted C₃-C₈ cycloalkyl, a partially unsaturated or aromatic carbocyclic group, or an optionally substituted saturated, partially unsaturated, or aromatic heterocyclic group; wherein at least one of A₁ and A₂ is an optionally substituted aromatic carbocyclic group or an optionally substituted aromatic heterocyclic group.

X and W are independently O, S, NR, or absent, where R is hydrogen, optionally substituted C₁-C₆ alkyl, or optionally substituted aryl(C₀-C₄ alkyl).

V is C₁-C₆ alkyl, C₂-C₆ alkenyl, C₃-C₇ cycloalkyl, or absent; and Y is C₁-C₆ alkyl, C₁-C₆ alkyl substituted with C₃-C₇ cycloalkyl, C₂-C₆ alkenyl, C₃-C₇ cycloalkyl, or absent; wherein when V is absent, W is absent; and Z is carbonyl, thiocarbonyl, imino, or C₁-C₆ alkylimino.

R₁ and R₂ are independently hydrogen or R₁ and R₂ are independently C₁-C₆ alkyl, C₂-C₆ alkenyl, or C₂-C₆ alkynyl, each of which is substituted with 0 to 3 substituents independently chosen from halogen, hydroxy, amino, C₁-C₄ alkoxy, C₁-C₂ haloalkyl, and C₁-C₂ haloalkoxy, or R₁ and R₂ are joined to form a 5- to 7-membered saturated or mono-unsaturated ring optionally containing one additional heteroatom chosen from N, S, and O, which 5- to 7-membered saturated or mono-unsaturated ring is substituted with 0 to 3 substituents independently chosen from halogen, hydroxy, amino, C₁-C₄ alkyl, C₁-C₄ alkoxy, mono- and di-(C₁-C₄alkyl)amino, C₁-C₂ haloalkyl, and C₁-C₂ haloalkoxy.

Suitable substituted aryl acylthiourea compounds are set forth in U.S. patent application Ser. No. 10/716,175, which is incorporated herein by reference in its entirety.

A compound of the present invention includes a compound identified by any of the methods of the present invention. In an embodiment, a compound of the present invention is a replicase complex defect inducer identified by any of the methods of the present invention, wherein said replicase complex defect inducer is a compound of Formula (II)

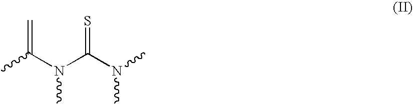

with the proviso that the replicase complex defect inducer is not a compound disclosed in Appendix A, Appendix B, or Appendix C hereto, Baltabaeva et al., Azerbaidzhanskii Kim. Zhur., 4: 97-99 (2000); Baltabaev et al., Khimiko-Farm. Zhur., 36(2): 24-26 (2002); Bloom et al., Biorganic & Med Chem. Lett., 13: 2929-2932 (2003); Daugulis et al., Latvijas Kimijas Zurnals, 6 :714-719 (1993); Douglass et al., JACS, 56 (3): 719-721 (1934); Douglass et al., JACS p. 1609 (July 1934); Du et al., Chemistry & Biology, 7(9): 733-742 (2000); Goerdeler et al., Chemische Berichte, 99(11): 3572-3581 (1966); Goerdeler et al., Liebigs Ann Chem 731: 120-141 (1970); Koscik et al., Collect. Czech. Chem. Comm., 48: 3315-3328 (1983); Kulka et al., Canadian J. Chem, 58: 2044-2048 (1980); Kutschy et al., Collect. Czech. Chem. Comm., 64(2): 348-362 (1999); Ludovici et al., Biorganic & Med Chem Lett, 11(17): 2225-2228 (2001); Matosiuk et al., Acta Polonine Pharm. Drug Research, 53(1): 75-77 (1996); Mishra et al., PharmaceuticaActa Helvetiae, 73: 215-218 (1998); Misra et al., J. fur Praktische Chemie 4 Reihe Band, 36: 256-259 (1967); Mitin et al., Fiziologicheski Aktivnye Veshchestva, 9:31-35 (1977); Mukmeneva et al., Russian J. Applied Chem., 67(4) (1994); Otazo-Sanchez et al., J. Chem. Soc. Perk. Trans., 2: 2211-221 (2001); Patel et al., Indian J. Heter. Chem., 12: 83-84 (2002); Patel et al., Oriental J. Chem, 18(3): 551-554 (2002); Praceus et al., Natruwissenschaften, 51(4): 94-5 (1964); Rashan et al., II Farmaco, 46(5): 677-683 (1991); Rasmussen et al., Synthesis, 6: 456-459 (June 1988); Reynaud et al., Chimie Therapeutique, 7: 421-424 (1966); Schuster, Zentralblatt fur Bakteriologie, Parasitenkunde, Infektionskranheitin un Hygiene Mikrobiolo. Der Landw., 133(7-8): 686-9 (1978); Sengupta et al., Indian J. Chem., 53(1): 203-204 (1976); Seth et al., Tet. Lett., 43: 7303-7306 (2002); Shearer et al., J. Med. Chem., 40(12): 1901-1905 (1997); Taniguchi et al., Chem. Pharm. Bull., 40(1): 240-244 (1992); Weinstein et al., Antibiotics and Chemotherapy, VII(8): 443-448 (1957); Matsuo et al., Chem. Pharm. Bull., 33(10): 4409-4421 (1985); U.S. Pat. No. 3,699,110; U.S. Pat. No. 3,966,968; U.S. Pat. No. 3,931,244; U.S. Pat. No. 4,082,765; U.S. Pat. No. 4,160,037; U.S. Pat. No. 4,338,257; U.S. Pat. No. 4,350,706; U.S. Pat. No. 4,533,676; U.S. Pat. No. 4,540,578; U.S. Pat. No. 4,602,109; U.S. Pat. No. 4,607,044; U.S. Pat. No. 4,638,088; U.S. Pat. No. 4,659,724; U.S. Pat. No. 4,659,736; U.S. Pat. No. 4,665,097; U.S. Pat. No. 4,707,478; U.S. Pat. No. 4,774,260; U.S. Pat. No. 4,868,215; U.S. Pat. No. 4,873,264; U.S. Pat. No. 4,880,838; U.S. Pat. No. 4,920,135; U.S. Pat. No. 5,001,266; U.S. Pat. No. 5,135,953; U.S. Pat. No. 5,166,180; U.S. Pat. No. 5,266,707; U.S. Pat. No. 5,344,842; U.S. Pat. No. 5,424,204; U.S. Pat. No. 5,437,996; U.S. Pat. No. 5,449,812; U.S. Pat. No. 5,589,365; U.S. Pat. No. 5,591,842; U.S. Pat. No. 5,656,642; U.S. Pat. No. 5,668,271; U.S. Pat. No. 5,723,409; U.S. Pat. No. 5,728,699; U.S. Pat. No. 5,760,058; U.S. Pat. No. 5,804,564; U.S. Pat. No. 5,840,917; U.S. Pat. No. 5,849,666; U.S. Pat. No. 5,874,615; U.S. Pat. No. 5,922,740; U.S. Pat. No. 6,060,484; U.S. Pat. No. 6,093,742; U.S. Pat. No. 6,133,258; U.S. Pat. No. 6,136,826; U.S. Pat. No. 6,169,092; U.S. Pat. No. 6,174,905; U.S. Pat. No. 6,207,715; U.S. Pat. No. 6,255,349; U.S. Pat. No. 6,268,387; U.S. Pat. No. 6,335,350; U.S. Pat. No. 6,399,657; U.S. Pat. No. 6,420,396; U.S. Pat. No. 6,541,485; U.S. Pat. No. 6,528,528; U.S. Pat. No. 6,610,715; U.S. Pat. No. 6,677,360; U.S. Pat. No. 6,677,372; U.S. Pat. No. 6,780,873; U.S. Publication No. 2001/0031874; U.S. Publication No. 2002/0016461; U.S. Publication No. 2002/0099210; U.S. Publication No. 2003/0109578; U.S. Publication No. 2003/0109579; U.S. Publication No. 2003/0125318; U.S. Publication No. 2003/0195231; U.S. Publication No. 2004/0009982; U.S. Publication No. 2004/0014754; U.S. Publication No. 2004/0029877; U.S. Publication No. 2004/0030132; U.S. Publication No. 2004/0132727; U.S. Publication No. 2004/0147535; U.S. Publication No. 2004/0147569; U.S. Publication No. 2004/0147741; U.S. Publication No. 2004/0162287; CN1183409; DE2303761; EP0518376; EP0728481; JP0603787; JP06287171; JP11335375; JP61106551; JP56025148; WO 1997/003976; WO 1997/011050; WO 1997/030047; WO 1998/042323; WO 1999/059586; WO 2000/035864; WO 2000/076518; WO 2001/021576; WO 2001/047890; WO 2001/047931; WO 2002/089783; WO 2002/090317; WO 2003/037869; WO 2003/097604; WO 2003/097605; WO 2003/099812; WO 2004/013102; or WO 2004/020416, all of which documents are herein incorporated by reference in their entireties and which documents, for example, are herein incorporated with regard to the compounds that they disclose.

In an embodiment, a compound of the present invention is a replicase complex defect inducer identified by the method of screening a test compound for replicase complex defect inducer activity comprising: providing a test compound; contacting the test compound with a cell expressing an HCV virion that comprises an NS3 protein with a mutation at or within about 15 angstroms of C16; and identifying the test compound as an inducer of an HCV replicase complex defect when the cell or HCV virion is resistant to the test compound, wherein said replicase complex defect inducer is a compound of Formula (II)

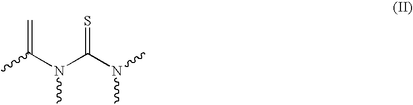

with the proviso that the replicase complex defect inducer is not a compound disclosed in Appendix A, Appendix B, or Appendix C hereto, Baltabaeva et al., Azerbaidzhanskii Kim. Zhur., 4: 97-99 (2000); Baltabaev et al., Khimiko-Farm. Zhur., 36(2): 24-26 (2002); Bloom et al., Biorganic & Med Chem Lett., 13: 2929-2932 (2003); Daugulis et al., Latvijas Kimijas Zurnals, 6 :714-719 (1993); Douglass et al., JACS, 56 (3): 719-721 (1934); Douglass et al., JACS p. 1609 (July 1934); Du et al., Chemistry & Biology, 7(9): 733-742 (2000); Goerdeler et al., Chemische Berichte, 99(11): 3572-3581 (1966); Goerdeler et al., Liebigs Ann Chem 731: 120-141 (1970); Koscik et al., Collect. Czech. Chem. Comm., 48: 3315-3328 (1983); Kulka et al., Canadian J. Chem, 58: 2044-2048 (1980); Kutschy et al., Collect. Czech. Chem. Comm., 64(2): 348-362 (1999); Ludovici et al., Biorganic & Med Chem Lett, 11(17): 2225-2228 (2001); Matosiuk et al., Acta Polonine Pharm. Drug Research, 53(1): 75-77 (1996); Mishra et al., PharmaceuticaActa Helvetiae, 73: 215-218 (1998); Misra et al., J. fur Praktische Chemie 4 Reihe Band, 36: 256-259 (1967); Mitin et al., Fiziologicheski Aktivnye Veshchestva, 9:31-35 (1977); Mukmeneva et al., Russian J. Applied Chem., 67(4) (1994); Otazo-Sanchez et al., J. Chem. Soc. Perk. Trans., 2: 2211-221 (2001); Patel et al., Indian J. Heter. Chem., 12: 83-84 (2002); Patel et al., Oriental J. Chem, 18(3): 551-554 (2002); Praceus et al., Natruwissenschaften, 51(4): 94-5 (1964); Rashan et al., II Farmaco, 46(5): 677-683 (1991); Rasmussen et al., Synthesis, 6: 456-459 (June 1988); Reynaud et al., Chimie Therapeutique, 7: 421-424 (1966); Schuster, Zentralblatt fur Bakteriologie, Parasitenkunde, Infektionskranheitin un Hygiene Mikrobiolo. Der Landw., 133(7-8): 686-9 (1978); Sengupta et al., Indian J. Chem., 53(1): 203-204 (1976); Seth et al., Tet. Lett., 43: 7303-7306 (2002); Shearer et al., J. Med. Chem., 40(12): 1901-1905 (1997); Taniguchi et al., Chem. Pharm. Bull., 40(1): 240-244 (1992); Weinstein et al., Antibiotics and Chemotherapy, VII(8): 443-448 (1957); Matsuo et al., Chem. Pharm. Bull., 33(10): 4409-4421 (1985); U.S. Pat. No. 3,699,110; U.S. Pat. No. 3,966,968; U.S. Pat. No. 3,931,244; U.S. Pat. No. 4,082,765; U.S. Pat. No. 4,160,037; U.S. Pat. No. 4,338,257; U.S. Pat. No. 4,350,706; U.S. Pat. No. 4,533,676; U.S. Pat. No. 4,540,578; U.S. Pat. No. 4,602,109; U.S. Pat. No. 4,607,044; U.S. Pat. No. 4,638,088; U.S. Pat. No. 4,659,724; U.S. Pat. No. 4,659,736; U.S. Pat. No. 4,665,097; U.S. Pat. No. 4,707,478; U.S. Pat. No. 4,774,260; U.S. Pat. No. 4,868,215; U.S. Pat. No. 4,873,264; U.S. Pat. No. 4,880,838; U.S. Pat. No. 4,920,135; U.S. Pat. No. 5,001,266; U.S. Pat. No. 5,135,953; U.S. Pat. No. 5,166,180; U.S. Pat. No. 5,266,707; U.S. Pat. No. 5,344,842; U.S. Pat. No. 5,424,204; U.S. Pat. No. 5,437,996; U.S. Pat. No. 5,449,812; U.S. Pat. No. 5,589,365; U.S. Pat. No. 5,591,842; U.S. Pat. No. 5,656,642; U.S. Pat. No. 5,668,271; U.S. Pat. No. 5,723,409; U.S. Pat. No. 5,728,699; U.S. Pat. No. 5,760,058; U.S. Pat. No. 5,804,564; U.S. Pat. No. 5,840,917; U.S. Pat. No. 5,849,666; U.S. Pat. No. 5,874,615; U.S. Pat. No. 5,922,740; U.S. Pat. No. 6,060,484; U.S. Pat. No. 6,093,742; U.S. Pat. No. 6,133,258; U.S. Pat. No. 6,136,826; U.S. Pat. No. 6,169,092; U.S. Pat. No. 6,174,905; U.S. Pat. No. 6,207,715; U.S. Pat. No. 6,255,349; U.S. Pat. No. 6,268,387; U.S. Pat. No. 6,335,350; U.S. Pat. No. 6,399,657; U.S. Pat. No. 6,420,396; U.S. Pat. No. 6,541,485; U.S. Pat. No. 6,528,528; U.S. Pat. No. 6,610,715; U.S. Pat. No. 6,677,360; U.S. Pat. No. 6,677,372; U.S. Pat. No. 6,780,873; U.S. Publication No. 2001/0031874; U.S. Publication No. 2002/0016461; U.S. Publication No. 2002/0099210; U.S. Publication No. 2003/0109578; U.S. Publication No. 2003/0109579; U.S. Publication No. 2003/0125318; U.S. Publication No. 2003/0195231; U.S. Publication No. 2004/0009982; U.S. Publication No. 2004/0014754; U.S. Publication No. 2004/0029877; U.S. Publication No. 2004/0030132; U.S. Publication No. 2004/0132727; U.S. Publication No. 2004/0138205; U.S. Publication No. 2004/0147535; U.S. Publication No. 2004/0147569; U.S. Publication No. 2004/0147741; U.S. Publication No. 2004/0162287; CN1183409; DE2303761; EP0518376; EP0728481; JP0603787; JP06287171; JP11335375; JP61106551; JP56025148; WO 1997/003976; WO 1997/011050; WO 1997/030047; WO 1998/042323; WO 1999/059586; WO 2000/035864; WO 2000/076518; WO 2001/021576; WO 2001/047890; WO 2001/047931; WO 2002/089783; WO 2002/090317; WO 2003/037869; WO 2003/097604; WO 2003/097605; WO 2003/099812; WO 2004/013102; WO 2004/020416; or WO 2004/046095, all of which documents are herein incorporated by reference in their entireties and which documents, for example, are herein incorporated with regard to the compounds that they disclose.

In an embodiment, a compound of the present invention is a replicase complex defect inducer identified by the method of screening a test compound for replicase complex defect inducer activity comprising: providing a test compound; contacting the test compound with a cell expressing an HCV replicon that comprises an NS3 protein with a mutation at or within about 15 angstroms of C16; and identifying the test compound as an inducer of an HCV replicase complex defect when the cell or HCV replicon is resistant to the test compound, wherein said replicase complex defect inducer is a compound of Formula (II)

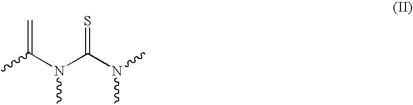

with the proviso that the replicase complex defect inducer is not a compound disclosed in Appendix A, Appendix B, or Appendix C hereto, Baltabaeva et al., Azerbaidzhanskii Kim. Zhur., 4: 97-99 (2000); Baltabaev et al., Khimiko-Farm. Zhur., 36(2): 24-26 (2002); Bloom et al., Biorganic & Med Chem Lett., 13: 2929-2932 (2003); Daugulis et al., Latvijas Kimijas Zurnals, 6 :714-719 (1993); Douglass et al., JACS, 56 (3): 719-721 (1934); Douglass et al., JACS p. 1609 (July 1934); Du et al., Chemistry & Biology, 7(9): 733-742 (2000); Goerdeler et al., Chemische Berichte, 99(11): 3572-3581 (1966); Goerdeler et al., Liebigs Ann Chem 731: 120-141 (1970); Koscik et al., Collect. Czech. Chem. Comm., 48: 3315-3328 (1983); Kulka et al., Canadian J. Chem, 58: 2044-2048 (1980); Kutschy et al., Collect. Czech. Chem. Comm., 64(2): 348-362 (1999); Ludovici et al., Biorganic & Med Chem Lett, 11(17): 2225-2228 (2001); Matosiuk et al., Acta Polonine Pharm. Drug Research, 53(1): 75-77 (1996); Mishra et al., Pharmaceutica Acta Helvetiae, 73: 215-218 (1998); Misra et al., J. fur Praktische Chemie 4 Reihe Band, 36: 256-259 (1967); Mitin et al., Fiziologicheski Aktivnye Veshchestva, 9:31-35 (1977); Mukmeneva et al., Russian J. Applied Chem., 67(4) (1994); Otazo-Sanchez et al., J. Chem. Soc. Perk. Trans., 2: 2211-221 (2001); Patel et al., Indian J. Heter. Chem., 12: 83-84 (2002); Patel et al., Oriental J. Chem, 18(3): 551-554 (2002); Praceus et al., Natruwissenschaften, 51(4): 94-5 (1964); Rashan et al., II Farmaco, 46(5): 677-683 (1991); Rasmussen et al., Synthesis, 6: 456-459 (June 1988); Reynaud et al., Chimie Therapeutique, 7: 421-424 (1966); Schuster, Zentralblatt fur Bakteriologie, Parasitenkunde, Infektionskranheitin un Hygiene Mikrobiolo. Der Landw., 133(7-8): 686-9 (1978); Sengupta et al., Indian J. Chem., 53(1): 203-204 (1976); Seth et al., Tet. Lett., 43: 7303-7306 (2002); Shearer et al., J. Med. Chem., 40(12): 1901-1905 (1997); Taniguchi et al., Chem. Pharm. Bull., 40(1): 240-244 (1992); Weinstein et al., Antibiotics and Chemotherapy, VII(8): 443-448 (1957); Matsuo et al., Chem. Pharm. Bull., 33(10): 4409-4421 (1985); U.S. Pat. No. 3,699,110; U.S. Pat. No. 3,966,968; U.S. Pat. No. 3,931,244; U.S. Pat. No. 4,082,765; U.S. Pat. No. 4,160,037; U.S. Pat. No. 4,338,257; U.S. Pat. No. 4,350,706; U.S. Pat. No. 4,533,676; U.S. Pat. No. 4,540,578; U.S. Pat. No. 4,602,109; U.S. Pat. No. 4,607,044; U.S. Pat. No. 4,638,088; U.S. Pat. No. 4,659,724; U.S. Pat. No. 4,659,736; U.S. Pat. No. 4,665,097; U.S. Pat. No. 4,707,478; U.S. Pat. No. 4,774,260; U.S. Pat. No. 4,868,215; U.S. Pat. No. 4,873,264; U.S. Pat. No. 4,880,838; U.S. Pat. No. 4,920,135; U.S. Pat. No. 5,001,266; U.S. Pat. No. 5,135,953; U.S. Pat. No. 5,166,180; U.S. Pat. No. 5,266,707; U.S. Pat. No. 5,344,842; U.S. Pat. No. 5,424,204; U.S. Pat. No. 5,437,996; U.S. Pat. No. 5,449,812; U.S. Pat. No. 5,589,365; U.S. Pat. No. 5,591,842; U.S. Pat. No. 5,656,642; U.S. Pat. No. 5,668,271; U.S. Pat. No. 5,723,409; U.S. Pat. No. 5,728,699; U.S. Pat. No. 5,760,058; U.S. Pat. No. 5,804,564; U.S. Pat. No. 5,840,917; U.S. Pat. No. 5,849,666; U.S. Pat. No. 5,874,615; U.S. Pat. No. 5,922,740; U.S. Pat. No. 6,060,484; U.S. Pat. No. 6,093,742; U.S. Pat. No. 6,133,258; U.S. Pat. No. 6,136,826; U.S. Pat. No. 6,169,092; U.S. Pat. No. 6,174,905; U.S. Pat. No. 6,207,715; U.S. Pat. No. 6,255,349; U.S. Pat. No. 6,268,387; U.S. Pat. No. 6,335,350; U.S. Pat. No. 6,399,657; U.S. Pat. No. 6,420,396; U.S. Pat. No. 6,541,485; U.S. Pat. No. 6,528,528; U.S. Pat. No. 6,610,715; U.S. Pat. No. 6,677,360; U.S. Pat. No. 6,677,372; U.S. Pat. No. 6,780,873; U.S. Publication No. 2001/0031874; U.S. Publication No. 2002/0016461; U.S. Publication No. 2002/0099210; U.S. Publication No. 2003/0109578; U.S. Publication No. 2003/0109579; U.S. Publication No. 2003/0125318; U.S. Publication No. 2003/0195231; U.S. Publication No. 2004/0009982; U.S. Publication No. 2004/0014754; U.S. Publication No. 2004/0029877; U.S. Publication No. 2004/0030132; U.S. Publication No. 2004/0132727; U.S. Publication No. 2004/0138205; U.S. Publication No. 2004/0147535; U.S. Publication No. 2004/0147569; U.S. Publication No. 2004/0147741; U.S. Publication No. 2004/0162287; CN1183409; DE2303761; EP0518376; EP0728481; JP0603787; JP06287171; JP11335375; JP61106551; JP56025148; WO 1997/003976; WO 1997/011050; WO 1997/030047; WO 1998/042323; WO 1999/059586; WO 2000/035864; WO 2000/076518; WO 2001/021576; WO 2001/047890; WO 2001/047931; WO 2002/089783; WO 2002/090317; WO 2003/037869; WO 2003/097604; WO 2003/097605; WO 2003/099812; WO 2004/013102; WO 2004/020416; or WO 2004/046095, all of which documents are herein incorporated by reference in their entireties and which documents, for example, are herein incorporated with regard to the compounds that they disclose.

In another embodiment, a compound of the present invention is a replicase complex defect inducer identified by the method of screening a test compound for replicase complex defect inducer activity comprising: providing a test compound; contacting the test compound with a cell expressing an isolated HCV replicase complex that comprises an NS3 protein with a mutation at or within about 15 angstroms of C16; and identifying the test compound as an inducer of an HCV replicase complex defect when the cell or HCV replicase complex is resistant to the test compound, wherein said replicase complex defect inducer is a compound of Formula (II)

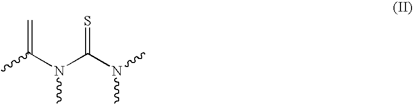

with the proviso that the replicase complex defect inducer is not a compound disclosed in Appendix A, Appendix B, or Appendix C hereto, Baltabaeva et al., Azerbaidzhanskii Kim. Zhur., 4: 97-99 (2000); Baltabaev et al., Khimiko-Farm. Zhur., 36(2): 24-26 (2002); Bloom et al., Biorganic & Med Chem Lett., 13: 2929-2932 (2003); Daugulis et al., Latvijas Kimijas Zurnals, 6 :714-719 (1993); Douglass et al., JACS, 56 (3): 719-721 (1934); Douglass et al., JACS p. 1609 (July 1934); Du et al., Chemistry & Biology, 7(9): 733-742 (2000); Goerdeler et al., Chemische Berichte, 99(11): 3572-3581 (1966); Goerdeler et al., Liebigs Ann Chem 731: 120-141 (1970); Koscik et al., Collect. Czech. Chem. Comm., 48: 3315-3328 (1983); Kulka et al., Canadian J. Chem, 58: 2044-2048 (1980); Kutschy et al., Collect. Czech. Chem. Comm., 64(2): 348-362 (1999); Ludovici et al., Biorganic & Med Chem Lett, 11(17): 2225-2228 (2001); Matosiuk et al., Acta Polonine Pharm. Drug Research, 53(1): 75-77 (1996); Mishra et al., PharmaceuticaActa Helvetiae, 73: 215-218 (1998); Misra et al., J. fur Praktische Chemie 4 Reihe Band, 36: 256-259 (1967); Mitin et al., Fiziologicheski Aktivnye Veshchestva, 9:31-35 (1977); Mukmeneva et al., Russian J. Applied Chem., 67(4) (1994); Otazo-Sanchez et al., J. Chem. Soc. Perk. Trans., 2: 2211-221 (2001); Patel et al., Indian J. Heter. Chem., 12: 83-84 (2002); Patel et al., Oriental J. Chem, 18(3): 551-554 (2002); Praceus et al., Natruwissenschaften, 51(4): 94-5 (1964); Rashan et al., II Farmaco, 46(5): 677-683 (1991); Rasmussen et al., Synthesis, 6: 456-459 (June 1988); Reynaud et al., Chimie Therapeutique, 7: 421-424 (1966); Schuster, Zentralblatt fur Bakteriologie, Parasitenkunde, Infektionskranheitin un Hygiene Mikrobiolo. Der Landw., 133(7-8): 686-9 (1978); Sengupta et al., Indian J. Chem., 53(1): 203-204 (1976); Seth et al., Tet. Lett., 43: 7303-7306 (2002); Shearer et al., J. Med. Chem., 40(12): 1901-1905 (1997); Taniguchi et al., Chem. Pharm. Bull., 40(1): 240-244 (1992); Weinstein et al., Antibiotics and Chemotherapy, VII(8): 443-448 (1957); Matsuo et al., Chem. Pharm. Bull., 33(10): 4409-4421 (1985); U.S. Pat. No. 3,699,110; U.S. Pat. No. 3,966,968; U.S. Pat. No. 3,931,244; U.S. Pat. No. 4,082,765; U.S. Pat. No. 4,160,037; U.S. Pat. No. 4,338,257; U.S. Pat. No. 4,350,706; U.S. Pat. No. 4,533,676; U.S. Pat. No. 4,540,578; U.S. Pat. No. 4,602,109; U.S. Pat. No. 4,607,044; U.S. Pat. No. 4,638,088; U.S. Pat. No. 4,659,724; U.S. Pat. No. 4,659,736; U.S. Pat. No. 4,665,097; U.S. Pat. No. 4,707,478; U.S. Pat. No. 4,774,260; U.S. Pat. No. 4,868,215; U.S. Pat. No. 4,873,264; U.S. Pat. No. 4,880,838; U.S. Pat. No. 4,920,135; U.S. Pat. No. 5,001,266; U.S. Pat. No. 5,135,953; U.S. Pat. No. 5,166,180; U.S. Pat. No. 5,266,707; U.S. Pat. No. 5,344,842; U.S. Pat. No. 5,424,204; U.S. Pat. No. 5,437,996; U.S. Pat. No. 5,449,812; U.S. Pat. No. 5,589,365; U.S. Pat. No. 5,591,842; U.S. Pat. No. 5,656,642; U.S. Pat. No. 5,668,271; U.S. Pat. No. 5,723,409; U.S. Pat. No. 5,728,699; U.S. Pat. No. 5,760,058; U.S. Pat. No. 5,804,564; U.S. Pat. No. 5,840,917; U.S. Pat. No. 5,849,666; U.S. Pat. No. 5,874,615; U.S. Pat. No. 5,922,740; U.S. Pat. No. 6,060,484; U.S. Pat. No. 6,093,742; U.S. Pat. No. 6,133,258; U.S. Pat. No. 6,136,826; U.S. Pat. No. 6,169,092; U.S. Pat. No. 6,174,905; U.S. Pat. No. 6,207,715; U.S. Pat. No. 6,255,349; U.S. Pat. No. 6,268,387; U.S. Pat. No. 6,335,350; U.S. Pat. No. 6,399,657; U.S. Pat. No. 6,420,396; U.S. Pat. No. 6,541,485; U.S. Pat. No. 6,528,528; U.S. Pat. No. 6,610,715; U.S. Pat. No. 6,677,360; U.S. Pat. No. 6,677,372; U.S. Pat. No. 6,780,873; U.S. Publication No. 2001/0031874; U.S. Publication No. 2002/0016461; U.S. Publication No. 2002/0099210; U.S. Publication No. 2003/0109578; U.S. Publication No. 2003/0109579; U.S. Publication No. 2003/0125318; U.S. Publication No. 2003/0195231; U.S. Publication No. 2004/0009982; U.S. Publication No. 2004/0014754; U.S. Publication No. 2004/0029877; U.S. Publication No. 2004/0030132; U.S. Publication No. 2004/0132727; U.S. Publication No. 2004/0138205; U.S. Publication No. 2004/0147535; U.S. Publication No. 2004/0147569; U.S. Publication No. 2004/0147741; U.S. Publication No. 2004/0162287; CN1183409; DE2303761; EP0518376; EP0728481; JP0603787; JP06287171; JP11335375; JP61106551; JP56025148; WO 1997/003976; WO 1997/011050; WO 1997/030047; WO 1998/042323; WO 1999/059586; WO 2000/035864; WO 2000/076518; WO 2001/021576; WO 2001/047890; WO 2001/047931; WO 2002/089783; WO 2002/090317; WO 2003/037869; WO 2003/097604; WO 2003/097605; WO 2003/099812; WO 2004/013102; WO 2004/020416; or WO 2004/046095.

In another embodiment, a compound of the present invention is a replicase complex defect inducer identified by the method of screening a test compound for replicase complex defect inducer activity comprising: providing a test compound; contacting the test compound with a cell expressing an isolated HCV polyprotein that comprises an NS3 protein with a mutation that is at or within about 15 angstroms of C16; and identifying the test compound as an inducer of an HCV replicase complex defect when the cell or HCV polyprotein is resistant to the test compound, wherein said replicase complex defect inducer is a compound of Formula (II)

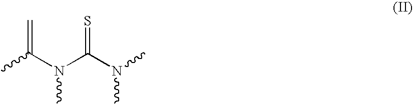

with the proviso that the replicase complex defect inducer is not a compound disclosed in Appendix A, Appendix B, or Appendix C hereto, Baltabaeva et al., Azerbaidzhanskii Kim. Zhur., 4: 97-99 (2000); Baltabaev et al., Khimiko-Farm. Zhur., 36(2): 24-26 (2002); Bloom et al., Biorganic & Med Chem Lett., 13: 2929-2932 (2003); Daugulis et al., Latvijas Kimijas Zurnals, 6 :714-719 (1993); Douglass et al., JACS, 56 (3): 719-721 (1934); Douglass et al., JACS p. 1609 (July 1934); Du et al., Chemistry & Biology, 7(9): 733-742 (2000); Goerdeler et al., Chemische Berichte, 99(11): 3572-3581 (1966); Goerdeler et al., Liebigs Ann Chem 731: 120-141 (1970); Koscik et al., Collect. Czech. Chem. Comm., 48: 3315-3328 (1983); Kulka et al., Canadian J. Chem, 58: 2044-2048 (1980); Kutschy et al., Collect. Czech. Chem. Comm., 64(2): 348-362 (1999); Ludovici et al., Biorganic & Med Chem Lett, 11(17): 2225-2228 (2001); Matosiuk et al., Acta Polonine Pharm. Drug Research, 53(1): 75-77 (1996); Mishra et al., Pharmaceutica Acta Helvetiae, 73: 215-218 (1998); Misra et al., J. fur Praktische Chemie 4 Reihe Band, 36: 256-259 (1967); Mitin et al., Fiziologicheski Aktivnye Veshchestva, 9:31-35 (1977); Mukmeneva et al., Russian J. Applied Chem., 67(4) (1994); Otazo-Sanchez et al., J. Chem. Soc. Perk. Trans., 2: 2211-221 (2001); Patel et al., Indian J. Heter. Chem., 12: 83-84 (2002); Patel et al., Oriental J. Chem, 18(3): 551-554 (2002); Praceus et al., Natruwissenschaften, 51(4): 94-5 (1964); Rashan et al., II Farmaco, 46(5): 677-683 (1991); Rasmussen et al., Synthesis, 6: 456-459 (June 1988); Reynaud et al., Chimie Therapeutique, 7: 421-424 (1966); Schuster, Zentralblatt fur Bakteriologie, Parasitenkunde, Infektionskranheitin un Hygiene Mikrobiolo. Der Landw., 133(7-8): 686-9 (1978); Sengupta et al., Indian Chem., 53(1): 203-204 (1976); Seth et al., Tet. Lett., 43: 7303-7306 (2002); Shearer et al., J. Med. Chem., 40(12): 1901-1905 (1997); Taniguchi et al., Chem. Pharm. Bull., 40(1): 240-244 (1992); Weinstein et al., Antibiotics and Chemotherapy, VII(8): 443-448 (1957); Matsuo et al., Chem. Pharm. Bull., 33(10): 4409-4421 (1985); U.S. Pat. No. 3,699,110; U.S. Pat. No. 3,966,968; U.S. Pat. No. 3,931,244; U.S. Pat. No. 4,082,765; U.S. Pat. No. 4,160,037; U.S. Pat. No. 4,338,257; U.S. Pat. No. 4,350,706; U.S. Pat. No. 4,533,676; U.S. Pat. No. 4,540,578; U.S. Pat. No. 4,602,109; U.S. Pat. No. 4,607,044; U.S. Pat. No. 4,638,088; U.S. Pat. No. 4,659,724; U.S. Pat. No. 4,659,736; U.S. Pat. No. 4,665,097; U.S. Pat. No. 4,707,478; U.S. Pat. No. 4,774,260; U.S. Pat. No. 4,868,215; U.S. Pat. No. 4,873,264; U.S. Pat. No. 4,880,838; U.S. Pat. No. 4,920,135; U.S. Pat. No. 5,001,266; U.S. Pat. No. 5,135,953; U.S. Pat. No. 5,166,180; U.S. Pat. No. 5,266,707; U.S. Pat. No. 5,344,842; U.S. Pat. No. 5,424,204; U.S. Pat. No. 5,437,996; U.S. Pat. No. 5,449,812; U.S. Pat. No. 5,589,365; U.S. Pat. No. 5,591,842; U.S. Pat. No. 5,656,642; U.S. Pat. No. 5,668,271; U.S. Pat. No. 5,723,409; U.S. Pat. No. 5,728,699; U.S. Pat. No. 5,760,058; U.S. Pat. No. 5,804,564; U.S. Pat. No. 5,840,917; U.S. Pat. No. 5,849,666; U.S. Pat. No. 5,874,615; U.S. Pat. No. 5,922,740; U.S. Pat. No. 6,060,484; U.S. Pat. No. 6,093,742; U.S. Pat. No. 6,133,258; U.S. Pat. No. 6,136,826; U.S. Pat. No. 6,169,092; U.S. Pat. No. 6,174,905; U.S. Pat. No. 6,207,715; U.S. Pat. No. 6,255,349; U.S. Pat. No. 6,268,387; U.S. Pat. No. 6,335,350; U.S. Pat. No. 6,399,657; U.S. Pat. No. 6,420,396; U.S. Pat. No. 6,541,485; U.S. Pat. No. 6,528,528; U.S. Pat. No. 6,610,715; U.S. Pat. No. 6,677,360; U.S. Pat. No. 6,677,372; U.S. Pat. No. 6,780,873; U.S. Publication No. 2001/0031874; U.S. Publication No. 2002/0016461; U.S. Publication No. 2002/0099210; U.S. Publication No. 2003/0109578; U.S. Publication No. 2003/0109579; U.S. Publication No. 2003/0125318; U.S. Publication No. 2003/0195231; U.S. Publication No. 2004/0009982; U.S. Publication No. 2004/0014754; U.S. Publication No. 2004/0029877; U.S. Publication No. 2004/0030132; U.S. Publication No. 2004/0132727; U.S. Publication No. 2004/0138205; U.S. Publication No. 2004/0147535; U.S. Publication No. 2004/0147569; U.S. Publication No. 2004/0147741; U.S. Publication No. 2004/0162287; CN1183409; DE2303761; EP0518376; EP0728481; JP0603787; JP06287171; JP11335375; JP61106551; JP56025148; WO 1997/003976; WO 1997/011050; WO 1997/030047; WO 1998/042323; WO 1999/059586; WO 2000/035864; WO 2000/076518; WO 2001/021576; WO 2001/047890; WO 2001/047931; WO 2002/089783; WO 2002/090317; WO 2003/037869; WO 2003/097604; WO 2003/097605; WO 2003/099812; WO 2004/013102; WO 2004/020416; or WO 2004/046095.

In another embodiment, a compound of the present invention is a replicase complex defect inducer identified by the method of screening a test compound for replicase complex defect inducer activity comprising: providing a test compound; contacting the test compound with a cell that expresses an HCV virion; and identifying the test compound as an inducer of an HCV replicase complex defect, wherein said replicase complex defect inducer is a compound of Formula (II)

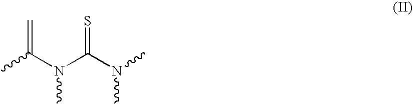

with the proviso that the replicase complex defect inducer is not a compound disclosed in Appendix A, Appendix B, or Appendix C hereto, Baltabaeva et al., Azerbaidzhanskii Kim. Zhur., 4: 97-99 (2000); Baltabaev et al., Khimiko-Farm. Zhur., 36(2): 24-26 (2002); Bloom et al., Biorganic & Med Chem Lett., 13: 2929-2932 (2003); Daugulis et al., Latvijas Kimijas Zurnals, 6 :714-719 (1993); Douglass et al., JACS, 56 (3): 719-721 (1934); Douglass et al., JACS p. 1609 (July 1934); Du et al., Chemistry & Biology, 7(9): 733-742 (2000); Goerdeler et al., Chemische Berichte, 99(11): 3572-3581 (1966); Goerdeler et al., Liebigs Ann Chem 731: 120-141 (1970); Koscik et al., Collect. Czech. Chem. Comm., 48: 3315-3328 (1983); Kulka et al., Canadian J. Chem, 58: 2044-2048 (1980); Kutschy et al., Collect. Czech. Chem. Comm., 64(2): 348-362 (1999); Ludovici et al., Biorganic & Med Chem Lett, 11(17): 2225-2228 (2001); Matosiuk et al., Acta Polonine Pharm. Drug Research, 53(1): 75-77 (1996); Mishra et al., Pharmaceutica Acta Helvetiae, 73: 215-218 (1998); Misra et al., J. fur Praktische Chemie 4 Reihe Band, 36: 256-259 (1967); Mitin et al., Fiziologicheski Aktivnye Veshchestva, 9:31-35 (1977); Mukmeneva et al., Russian J. Applied Chem., 67(4) (1994); Otazo-Sanchez et al., J. Chem. Soc. Perk. Trans., 2: 2211-221 (2001); Patel et al., Indian J. Heter. Chem., 12: 83-84 (2002); Patel et al., Oriental J. Chem, 18(3): 551-554 (2002); Praceus et al., Natruwissenschaften, 51(4): 94-5 (1964); Rashan et al., II Farmaco, 46(5): 677-683 (1991); Rasmussen et al., Synthesis, 6: 456-459 (June 1988); Reynaud et al., Chimie Therapeutique, 7: 421-424 (1966); Schuster, Zentralblatt fur Bakteriologie, Parasitenkunde, Infektionskranheitin un Hygiene Mikrobiolo. Der Landw., 133(7-8): 686-9 (1978); Sengupta et al., Indian J. Chem., 53(1): 203-204 (1976); Seth et al., Tet. Lett., 43: 7303-7306 (2002); Shearer et al., J. Med. Chem., 40(12): 1901-1905 (1997); Taniguchi et al., Chem. Pharm. Bull., 40(1): 240-244 (1992); Weinstein et al., Antibiotics and Chemotherapy, VII(8): 443-448 (1957); Matsuo et al., Chem. Pharm. Bull., 33(10): 4409-4421 (1985); U.S. Pat. No. 3,699,110; U.S. Pat. No. 3,966,968; U.S. Pat. No. 3,931,244; U.S. Pat. No. 4,082,765; U.S. Pat. No. 4,160,037; U.S. Pat. No. 4,338,257; U.S. Pat. No. 4,350,706; U.S. Pat. No. 4,533,676; U.S. Pat. No. 4,540,578; U.S. Pat. No. 4,602,109; U.S. Pat. No. 4,607,044; U.S. Pat. No. 4,638,088; U.S. Pat. No. 4,659,724; U.S. Pat. No. 4,659,736; U.S. Pat. No. 4,665,097; U.S. Pat. No. 4,707,478; U.S. Pat. No. 4,774,260; U.S. Pat. No. 4,868,215; U.S. Pat. No. 4,873,264; U.S. Pat. No. 4,880,838; U.S. Pat. No. 4,920,135; U.S. Pat. No. 5,001,266; U.S. Pat. No. 5,135,953; U.S. Pat. No. 5,166,180; U.S. Pat. No. 5,266,707; U.S. Pat. No. 5,344,842; U.S. Pat. No. 5,424,204; U.S. Pat. No. 5,437,996; U.S. Pat. No. 5,449,812; U.S. Pat. No. 5,589,365; U.S. Pat. No. 5,591,842; U.S. Pat. No. 5,656,642; U.S. Pat. No. 5,668,271; U.S. Pat. No. 5,723,409; U.S. Pat. No. 5,728,699; U.S. Pat. No. 5,760,058; U.S. Pat. No. 5,804,564; U.S. Pat. No. 5,840,917; U.S. Pat. No. 5,849,666; U.S. Pat. No. 5,874,615; U.S. Pat. No. 5,922,740; U.S. Pat. No. 6,060,484; U.S. Pat. No. 6,093,742; U.S. Pat. No. 6,133,258; U.S. Pat. No. 6,136,826; U.S. Pat. No. 6,169,092; U.S. Pat. No. 6,174,905; U.S. Pat. No. 6,207,715; U.S. Pat. No. 6,255,349; U.S. Pat. No. 6,268,387; U.S. Pat. No. 6,335,350; U.S. Pat. No. 6,399,657; U.S. Pat. No. 6,420,396; U.S. Pat. No. 6,541,485; U.S. Pat. No. 6,528,528; U.S. Pat. No. 6,610,715; U.S. Pat. No. 6,677,360; U.S. Pat. No. 6,677,372; U.S. Pat. No. 6,780,873; U.S. Publication No. 2001/0031874; U.S. Publication No. 2002/0016461; U.S. Publication No. 2002/0099210; U.S. Publication No. 2003/0109578; U.S. Publication No. 2003/0109579; U.S. Publication No. 2003/0125318; U.S. Publication No. 2003/0195231; U.S. Publication No. 2004/0009982; U.S. Publication No. 2004/0014754; U.S. Publication No. 2004/0029877; U.S. Publication No. 2004/0030132; U.S. Publication No. 2004/0132727; U.S. Publication No. 2004/0138205; U.S. Publication No. 2004/0147535; U.S. Publication No. 2004/0147569; U.S. Publication No. 2004/0147741; U.S. Publication No. 2004/0162287; CN1183409; DE2303761; EP0518376; EP0728481; JP0603787; JP06287171; JP11335375; JP61106551; JP56025148; WO 1997/003976; WO 1997/011050; WO 1997/030047; WO 1998/042323; WO 1999/059586; WO 2000/035864; WO 2000/076518; WO 2001/021576; WO 2001/047890; WO 2001/047931; WO 2002/089783; WO 2002/090317; WO 2003/037869; WO 2003/097604; WO 2003/097605; WO 2003/099812; WO 2004/013102; WO 2004/020416; or WO 2004/046095.

In another embodiment, a compound of the present invention is a replicase complex defect inducer identified by the method of screening a test compound for replicase complex defect inducer activity comprising: providing a test compound; contacting the test compound with a cell that expresses an HCV replicon; and identifying the test compound as an inducer of an HCV replicase complex defect, wherein said replicase complex defect inducer is a compound of Formula (II)

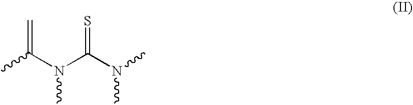

with the proviso that the replicase complex defect inducer is not a compound disclosed in Appendix A, Appendix B, or Appendix C hereto, Baltabaeva et al., Azerbaidzhanskii Kim. Zhur., 4: 97-99 (2000); Baltabaev et al., Khimiko-Farm. Zhur., 36(2): 24-26 (2002); Bloom et al., Biorganic & Med Chem Lett., 13: 2929-2932 (2003); Daugulis et al., Latvijas Kimijas Zurnals, 6 :714-719 (1993); Douglass et al., JACS, 56 (3): 719-721 (1934); Douglass et al., JACS p. 1609 (July 1934); Du et al., Chemistry & Biology, 7(9): 733-742 (2000); Goerdeler et al., Chemische Berichte, 99(11): 3572-3581 (1966); Goerdeler et al., Liebigs Ann Chem 731: 120-141 (1970); Koscik et al., Collect. Czech. Chem. Comm., 48: 3315-3328 (1983); Kulka et al., Canadian J. Chem, 58: 2044-2048 (1980); Kutschy et al., Collect. Czech. Chem. Comm., 64(2): 348-362 (1999); Ludovici et al., Biorganic & Med Chem Lett, 11(17): 2225-2228 (2001); Matosiuk et al., Acta Polonine Pharm. Drug Research, 53(1): 75-77 (1996); Mishra et al., Pharmaceutica Acta Helvetiae, 73: 215-218 (1998); Misra et al., J. fur Praktische Chemie 4 Reihe Band, 36: 256-259 (1967); Mitin et al., Fiziologicheski Aktivnye Veshchestva, 9:31-35 (1977); Mukmeneva et al., Russian J. Applied Chem., 67(4) (1994); Otazo-Sanchez et al., J. Chem. Soc. Perk. Trans., 2: 2211-221 (2001); Patel et al., Indian J. Heter. Chem., 12: 83-84 (2002); Patel et al., Oriental J. Chem, 18(3): 551-554 (2002); Praceus et al., Natruwissenschaften, 51(4): 94-5 (1964); Rashan et al., II Farmaco, 46(5): 677-683 (1991); Rasmussen et al., Synthesis, 6: 456-459 (June 1988); Reynaud et al., Chimie Therapeutique, 7: 421-424 (1966); Schuster, Zentralblatt fur Bakteriologie, Parasitenkunde, Infektionskranheitin un Hygiene Mikrobiolo. Der Landw., 133(7-8): 686-9 (1978); Sengupta et al., Indian J. Chem., 53(1): 203-204 (1976); Seth et al., Tet. Lett., 43: 7303-7306 (2002); Shearer et al., J. Med. Chem., 40(12): 1901-1905 (1997); Taniguchi et al., Chem. Pharm. Bull., 40(1): 240-244 (1992); Weinstein et al., Antibiotics and Chemotherapy, VII(8): 443-448 (1957); Matsuo et al., Chem. Pharm. Bull., 33(10): 4409-4421 (1985); U.S. Pat. No. 3,699,110; U.S. Pat. No. 3,966,968; U.S. Pat. No. 3,931,244; U.S. Pat. No. 4,082,765; U.S. Pat. No. 4,160,037; U.S. Pat. No. 4,338,257; U.S. Pat. No. 4,350,706; U.S. Pat. No. 4,533,676; U.S. Pat. No. 4,540,578; U.S. Pat. No. 4,602,109; U.S. Pat. No. 4,607,044; U.S. Pat. No. 4,638,088; U.S. Pat. No. 4,659,724; U.S. Pat. No. 4,659,736; U.S. Pat. No. 4,665,097; U.S. Pat. No. 4,707,478; U.S. Pat. No. 4,774,260; U.S. Pat. No. 4,868,215; U.S. Pat. No. 4,873,264; U.S. Pat. No. 4,880,838; U.S. Pat. No. 4,920,135; U.S. Pat. No. 5,001,266; U.S. Pat. No. 5,135,953; U.S. Pat. No. 5,166,180; U.S. Pat. No. 5,266,707; U.S. Pat. No. 5,344,842; U.S. Pat. No. 5,424,204; U.S. Pat. No. 5,437,996; U.S. Pat. No. 5,449,812; U.S. Pat. No. 5,589,365; U.S. Pat. No. 5,591,842; U.S. Pat. No. 5,656,642; U.S. Pat. No. 5,668,271; U.S. Pat. No. 5,723,409; U.S. Pat. No. 5,728,699; U.S. Pat. No. 5,760,058; U.S. Pat. No. 5,804,564; U.S. Pat. No. 5,840,917; U.S. Pat. No. 5,849,666; U.S. Pat. No. 5,874,615; U.S. Pat. No. 5,922,740; U.S. Pat. No. 6,060,484; U.S. Pat. No. 6,093,742; U.S. Pat. No. 6,133,258; U.S. Pat. No. 6,136,826; U.S. Pat. No. 6,169,092; U.S. Pat. No. 6,174,905; U.S. Pat. No. 6,207,715; U.S. Pat. No. 6,255,349; U.S. Pat. No. 6,268,387; U.S. Pat. No. 6,335,350; U.S. Pat. No. 6,399,657; U.S. Pat. No. 6,420,396; U.S. Pat. No. 6,541,485; U.S. Pat. No. 6,528,528; U.S. Pat. No. 6,610,715; U.S. Pat. No. 6,677,360; U.S. Pat. No. 6,677,372; U.S. Pat. No. 6,780,873; U.S. Publication No. 2001/0031874; U.S. Publication No. 2002/0016461; U.S. Publication No. 2002/0099210; U.S. Publication No. 2003/0109578; U.S. Publication No. 2003/0109579; U.S. Publication No. 2003/0125318; U.S. Publication No. 2003/0195231; U.S. Publication No. 2004/0009982; U.S. Publication No. 2004/0014754; U.S. Publication No. 2004/0029877; U.S. Publication No. 2004/0030132; U.S. Publication No. 2004/0132727; U.S. Publication No. 2004/0138205; U.S. Publication No. 2004/0147535; U.S. Publication No. 2004/0147569; U.S. Publication No. 2004/0147741; U.S. Publication No. 2004/0162287; CN1183409; DE2303761; EP0518376; EP0728481; JP0603787; JP06287171; JP11335375; JP61106551; JP56025148; WO 1997/003976; WO 1997/011050; WO 1997/030047; WO 1998/042323; WO 1999/059586; WO 2000/035864; WO 2000/076518; WO 2001/021576; WO 2001/047890; WO 2001/047931; WO 2002/089783; WO 2002/090317; WO 2003/037869; WO 2003/097604; WO 2003/097605; WO 2003/099812; WO 2004/013102; WO 2004/020416; or WO 2004/046095.

In another embodiment, a compound of the present invention is a replicase complex defect inducer identified by the method of screening a test compound for replicase complex defect inducer activity comprising: providing a test compound; contacting the test compound with a cell that expresses an isolated HCV replicase complex, and identifying the test compound as an inducer of an HCV replicase complex defect, wherein said replicase complex defect inducer is a compound of Formula (II)

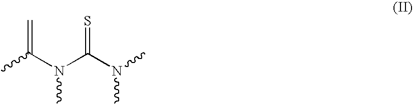

with the proviso that the replicase complex defect inducer is not a compound disclosed in Appendix A, Appendix B, or Appendix C hereto, Baltabaeva et al., Azerbaidzhanskii Kim. Zhur., 4: 97-99 (2000); Baltabaev et al., Khimiko-Farm. Zhur., 36(2): 24-26 (2002); Bloom et al., Biorganic & Med Chem Lett., 13: 2929-2932 (2003); Daugulis et al., Latvijas Kimijas Zurnals, 6 :714-719 (1993); Douglass et al., JACS, 56 (3): 719-721 (1934); Douglass et al., JACS p. 1609 (July 1934); Du et al., Chemistry & Biology, 7(9): 733-742 (2000); Goerdeler et al., Chemische Berichte, 99(11): 3572-3581 (1966); Goerdeler et al., Liebigs Ann Chem 731: 120-141 (1970); Koscik et al., Collect. Czech. Chem. Comm., 48: 3315-3328 (1983); Kulka et al., Canadian J. Chem, 58: 2044-2048 (1980); Kutschy et al., Collect. Czech. Chem. Comm., 64(2): 348-362 (1999); Ludovici et al., Biorganic & Med Chem Lett, 11(17): 2225-2228 (2001); Matosiuk et al., Acta Polonine Pharm. Drug Research, 53(1): 75-77 (1996); Mishra et al., PharmaceuticaActa Helvetiae, 73: 215-218 (1998); Misra et al., J. fur Praktische Chemie 4 Reihe Band, 36: 256-259 (1967); Mitin et al., Fiziologicheski Aktivnye Veshchestva, 9:31-35 (1977); Mukmeneva et al., Russian J. Applied Chem., 67(4) (1994); Otazo-Sanchez et al., J. Chem. Soc. Perk. Trans., 2: 2211-221 (2001); Patel et al., Indian J. Heter. Chem., 12: 83-84 (2002); Patel et al., Oriental J. Chem, 18(3): 551-554 (2002); Praceus et al., Natruwissenschaften, 51(4): 94-5 (1964); Rashan et al., II Farmaco, 46(5): 677-683 (1991); Rasmussen et al., Synthesis, 6: 456-459 (June 1988); Reynaud et al., Chimie Therapeutique, 7: 421-424 (1966); Schuster, Zentralblatt fur Bakteriologie, Parasitenkunde, Infektionskranheitin un Hygiene Mikrobiolo. Der Landw., 133(7-8): 686-9 (1978); Sengupta et al., Indian J. Chem., 53(1): 203-204 (1976); Seth et al., Tet. Lett., 43: 7303-7306 (2002); Shearer et al., J. Med. Chem., 40(12): 1901-1905 (1997); Taniguchi et al., Chem. Pharm. Bull., 40(1): 240-244 (1992); Weinstein et al., Antibiotics and Chemotherapy, VII(8): 443-448 (1957); Matsuo et al., Chem. Pharm. Bull., 33(10): 4409-4421 (1985); U.S. Pat. No. 3,699,110; U.S. Pat. No. 3,966,968; U.S. Pat. No. 3,931,244; U.S. Pat. No. 4,082,765; U.S. Pat. No. 4,160,037; U.S. Pat. No. 4,338,257; U.S. Pat. No. 4,350,706; U.S. Pat. No. 4,533,676; U.S. Pat. No. 4,540,578; U.S. Pat. No. 4,602,109; U.S. Pat. No. 4,607,044; U.S. Pat. No. 4,638,088; U.S. Pat. No. 4,659,724; U.S. Pat. No. 4,659,736; U.S. Pat. No. 4,665,097; U.S. Pat. No. 4,707,478; U.S. Pat. No. 4,774,260; U.S. Pat. No. 4,868,215; U.S. Pat. No. 4,873,264; U.S. Pat. No. 4,880,838; U.S. Pat. No. 4,920,135; U.S. Pat. No. 5,001,266; U.S. Pat. No. 5,135,953; U.S. Pat. No. 5,166,180; U.S. Pat. No. 5,266,707; U.S. Pat. No. 5,344,842; U.S. Pat. No. 5,424,204; U.S. Pat. No. 5,437,996; U.S. Pat. No. 5,449,812; U.S. Pat. No. 5,589,365; U.S. Pat. No. 5,591,842; U.S. Pat. No. 5,656,642; U.S. Pat. No. 5,668,271; U.S. Pat. No. 5,723,409; U.S. Pat. No. 5,728,699; U.S. Pat. No. 5,760,058; U.S. Pat. No. 5,804,564; U.S. Pat. No. 5,840,917; U.S. Pat. No. 5,849,666; U.S. Pat. No. 5,874,615; U.S. Pat. No. 5,922,740; U.S. Pat. No. 6,060,484; U.S. Pat. No. 6,093,742; U.S. Pat. No. 6,133,258; U.S. Pat. No. 6,136,826; U.S. Pat. No. 6,169,092; U.S. Pat. No. 6,174,905; U.S. Pat. No. 6,207,715; U.S. Pat. No. 6,255,349; U.S. Pat. No. 6,268,387; U.S. Pat. No. 6,335,350; U.S. Pat. No. 6,399,657; U.S. Pat. No. 6,420,396; U.S. Pat. No. 6,541,485; U.S. Pat. No. 6,528,528; U.S. Pat. No. 6,610,715; U.S. Pat. No. 6,677,360; U.S. Pat. No. 6,677,372; U.S. Pat. No. 6,780,873; U.S. Publication No. 2001/0031874; U.S. Publication No. 2002/0016461; U.S. Publication No. 2002/0099210; U.S. Publication No. 2003/0109578; U.S. Publication No. 2003/0109579; U.S. Publication No. 2003/0125318; U.S. Publication No. 2003/0195231; U.S. Publication No. 2004/0009982; U.S. Publication No. 2004/0014754; U.S. Publication No. 2004/0029877; U.S. Publication No. 2004/0030132; U.S. Publication No. 2004/0132727; U.S. Publication No. 2004/0138205; U.S. Publication No. 2004/0147535; U.S. Publication No. 2004/0147569; U.S. Publication No. 2004/0147741; U.S. Publication No. 2004/0162287; CN1183409; DE2303761; EP0518376; EP0728481; JP0603787; JP06287171; JP11335375; JP61106551; JP56025148; WO 1997/003976; WO 1997/011050; WO 1997/030047; WO 1998/042323; WO 1999/059586; WO 2000/035864; WO 2000/076518; WO 2001/021576; WO 2001/047890; WO 2001/047931; WO 2002/089783; WO 2002/090317; WO 2003/037869; WO 2003/097604; WO 2003/097605; WO 2003/099812; WO 2004/013102; WO 2004/020416; or WO 2004/046095.

In another embodiment, a compound of the present invention is a replicase complex defect inducer identified by the method of screening a test compound for replicase complex defect inducer activity comprising: providing a test compound; contacting the test compound with a cell that expresses an isolated HCV polyprotein or fragment thereof; and identifying the test compound as an inducer of an HCV replicase complex defect, wherein said replicase complex defect inducer is a compound of Formula (II)

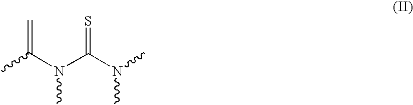

with the proviso that the replicase complex defect inducer is not a compound disclosed in Appendix A, Appendix B, or Appendix C hereto, Baltabaeva et al., Azerbaidzhanskii Kim. Zhur., 4: 97-99 (2000); Baltabaev et al., Khimiko-Farm. Zhur., 36(2): 24-26 (2002); Bloom et al., Biorganic & Med Chem Lett., 13: 2929-2932 (2003); Daugulis et al., Latvijas Kimijas Zurnals, 6 :714-719 (1993); Douglass et al., JACS, 56 (3): 719-721 (1934); Douglass et al., JACS p. 1609 (July 1934); Du et al., Chemistry & Biology, 7(9): 733-742 (2000); Goerdeler et al., Chemische Berichte, 99(11): 3572-3581 (1966); Goerdeler et al., Liebigs Ann Chem 731: 120-141 (1970); Koscik et al., Collect. Czech. Chem. Comm., 48: 3315-3328 (1983); Kulka et al., Canadian J. Chem, 58: 2044-2048 (1980); Kutschy et al., Collect. Czech. Chem. Comm., 64(2): 348-362 (1999); Ludovici et al., Biorganic & Med Chem Lett, 11(17): 2225-2228 (2001); Matosiuk et al., Acta Polonine Pharm. Drug Research, 53(1): 75-77 (1996); Mishra et al., Pharmaceutica Acta Helvetiae, 73: 215-218 (1998); Misra et al., J. fur Praktische Chemie 4 Reihe Band, 36: 256-259 (1967); Mitin et al., Fiziologicheski Aktivnye Veshchestva, 9:31-35 (1977); Mukmeneva et al., Russian J. Applied Chem., 67(4) (1994); Otazo-Sanchez et al., J. Chem. Soc. Perk. Trans., 2: 2211-221 (2001); Patel et al., Indian J. Heter. Chem., 12: 83-84 (2002); Patel et al., Oriental J. Chem, 18(3): 551-554 (2002); Praceus et al., Natruwissenschaften, 51(4): 94-5 (1964); Rashan et al., II Farmaco, 46(5): 677-683 (1991); Rasmussen et al., Synthesis, 6: 456-459 (June 1988); Reynaud et al., Chimie Therapeutique, 7: 421-424 (1966); Schuster, Zentralblatt fur Bakteriologie, Parasitenkunde, Infektionskranheitin un Hygiene Mikrobiolo. Der Landw., 133(7-8): 686-9 (1978); Sengupta et al., Indian J. Chem., 53(1): 203-204 (1976); Seth et al., Tet. Lett., 43: 7303-7306 (2002); Shearer et al., J. Med. Chem., 40(12): 1901-1905 (1997); Taniguchi et al., Chem. Pharm. Bull., 40(1): 240-244 (1992); Weinstein et al., Antibiotics and Chemotherapy, VII(8): 443-448 (1957); Matsuo et al., Chem. Pharm. Bull., 33(10): 4409-4421 (1985); U.S. Pat. No. 3,699,110; U.S. Pat. No. 3,966,968; U.S. Pat. No. 3,931,244; U.S. Pat. No. 4,082,765; U.S. Pat. No. 4,160,037; U.S. Pat. No. 4,338,257; U.S. Pat. No. 4,350,706; U.S. Pat. No. 4,533,676; U.S. Pat. No. 4,540,578; U.S. Pat. No. 4,602,109; U.S. Pat. No. 4,607,044; U.S. Pat. No. 4,638,088; U.S. Pat. No. 4,659,724; U.S. Pat. No. 4,659,736; U.S. Pat. No. 4,665,097; U.S. Pat. No. 4,707,478; U.S. Pat. No. 4,774,260; U.S. Pat. No. 4,868,215; U.S. Pat. No. 4,873,264; U.S. Pat. No. 4,880,838; U.S. Pat. No. 4,920,135; U.S. Pat. No. 5,001,266; U.S. Pat. No. 5,135,953; U.S. Pat. No. 5,166,180; U.S. Pat. No. 5,266,707; U.S. Pat. No. 5,344,842; U.S. Pat. No. 5,424,204; U.S. Pat. No. 5,437,996; U.S. Pat. No. 5,449,812; U.S. Pat. No. 5,589,365; U.S. Pat. No. 5,591,842; U.S. Pat. No. 5,656,642; U.S. Pat. No. 5,668,271; U.S. Pat. No. 5,723,409; U.S. Pat. No. 5,728,699; U.S. Pat. No. 5,760,058; U.S. Pat. No. 5,804,564; U.S. Pat. No. 5,840,917; U.S. Pat. No. 5,849,666; U.S. Pat. No. 5,874,615; U.S. Pat. No. 5,922,740; U.S. Pat. No. 6,060,484; U.S. Pat. No. 6,093,742; U.S. Pat. No. 6,133,258; U.S. Pat. No. 6,136,826; U.S. Pat. No. 6,169,092; U.S. Pat. No. 6,174,905; U.S. Pat. No. 6,207,715; U.S. Pat. No. 6,255,349; U.S. Pat. No. 6,268,387; U.S. Pat. No. 6,335,350; U.S. Pat. No. 6,399,657; U.S. Pat. No. 6,420,396; U.S. Pat. No. 6,541,485; U.S. Pat. No. 6,528,528; U.S. Pat. No. 6,610,715; U.S. Pat. No. 6,677,360; U.S. Pat. No. 6,677,372; U.S. Pat. No. 6,780,873; U.S. Publication No. 2001/0031874; U.S. Publication No. 2002/0016461; U.S. Publication No. 2002/0099210; U.S. Publication No. 2003/0109578; U.S. Publication No. 2003/0109579; U.S. Publication No. 2003/0125318; U.S. Publication No. 2003/0195231; U.S. Publication No. 2004/0009982; U.S. Publication No. 2004/0014754; U.S. Publication No. 2004/0029877; U.S. Publication No. 2004/0030132; U.S. Publication No. 2004/0132727; U.S. Publication No. 2004/0138205; U.S. Publication No. 2004/0147535; U.S. Publication No. 2004/0147569; U.S. Publication No. 2004/0147741; U.S. Publication No. 2004/0162287; CN1183409; DE2303761; EP0518376; EP0728481; JP0603787; JP06287171; JP11335375; JP61106551; JP56025148; WO 1997/003976; WO 1997/011050; WO 1997/030047; WO 1998/042323; WO 1999/059586; WO 2000/035864; WO 2000/076518; WO 2001/021576; WO 2001/047890; WO 2001/047931; WO 2002/089783; WO 2002/090317; WO 2003/037869; WO 2003/097604; WO 2003/097605; WO 2003/099812; WO 2004/013102; WO 2004/020416; or WO 2004/046095.

In another embodiment, a compound of the present invention is a replicase complex defect inducer identified by the method of screening a test compound for replicase complex defect inducer activity comprising: providing a test compound; contacting the test compound with an HCV virion; and identifying the test compound as an inducer of an HCV replicase complex defect when the level of p14 protein is increased, wherein said replicase complex defect inducer is a compound of Formula (II)

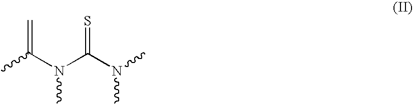

with the proviso that the replicase complex defect inducer is not a compound disclosed in Appendix A, Appendix B, or Appendix C hereto, Baltabaeva et al., Azerbaidzhanskii Kim. Zhur., 4: 97-99 (2000); Baltabaev et al., Khimiko-Farm. Zhur., 36(2): 24-26 (2002); Bloom et al., Biorganic & Med Chem Lett., 13: 2929-2932 (2003); Daugulis et al., Latvijas Kimijas Zurnals, 6 :714-719 (1993); Douglass et al., JACS, 56 (3): 719-721 (1934); Douglass et al., JACS p. 1609 (July 1934); Du et al., Chemistry & Biology, 7(9): 733-742 (2000); Goerdeler et al., Chemische Berichte, 99(11): 3572-3581 (1966); Goerdeler et al., Liebigs Ann Chem 731: 120-141 (1970); Koscik et al., Collect. Czech. Chem. Comm., 48: 3315-3328 (1983); Kulka et al., Canadian J. Chem, 58: 2044-2048 (1980); Kutschy et al., Collect. Czech. Chem. Comm., 64(2): 348-362 (1999); Ludovici et al., Biorganic & Med Chem Lett, 11(17): 2225-2228 (2001); Matosiuk et al., Acta Polonine Pharm. Drug Research, 53(1): 75-77 (1996); Mishra et al., Pharmaceutica Acta Helvetiae, 73: 215-218 (1998); Misra et al., J. fur Praktische Chemie 4 Reihe Band, 36: 256-259 (1967); Mitin et al., Fiziologicheski Aktivnye Veshchestva, 9:31-35 (1977); Mukmeneva et al., Russian J. Applied Chem., 67(4) (1994); Otazo-Sanchez et al., J. Chem. Soc. Perk. Trans., 2: 2211-221 (2001); Patel et al., Indian J. Heter. Chem., 12: 83-84 (2002); Patel et al., Oriental J. Chem, 18(3): 551-554 (2002); Praceus et al., Natruwissenschaften, 51(4): 94-5 (1964); Rashan et al., II Farmaco, 46(5): 677-683 (1991); Rasmussen et al., Synthesis, 6: 456-459 (June 1988); Reynaud et al., Chimie Therapeutique, 7: 421-424 (1966); Schuster, Zentralblatt fur Bakteriologie, Parasitenkunde, Infektionskranheitin un Hygiene Mikrobiolo. Der Landw., 133(7-8): 686-9 (1978); Sengupta et al., Indian J. Chem., 53(1): 203-204 (1976); Seth et al., Tet. Lett., 43: 7303-7306 (2002); Shearer et al., J. Med. Chem., 40(12): 1901-1905 (1997); Taniguchi et al., Chem. Pharm. Bull., 40(1): 240-244 (1992); Weinstein et al., Antibiotics and Chemotherapy, VII(8): 443-448 (1957); Matsuo et al., Chem. Pharm. Bull., 33(10): 4409-4421 (1985); U.S. Pat. No. 3,699,110; U.S. Pat. No. 3,966,968; U.S. Pat. No. 3,931,244; U.S. Pat. No. 4,082,765; U.S. Pat. No. 4,160,037; U.S. Pat. No. 4,338,257; U.S. Pat. No. 4,350,706; U.S. Pat. No. 4,533,676; U.S. Pat. No. 4,540,578; U.S. Pat. No. 4,602,109; U.S. Pat. No. 4,607,044; U.S. Pat. No. 4,638,088; U.S. Pat. No. 4,659,724; U.S. Pat. No. 4,659,736; U.S. Pat. No. 4,665,097; U.S. Pat. No. 4,707,478; U.S. Pat. No. 4,774,260; U.S. Pat. No. 4,868,215; U.S. Pat. No. 4,873,264; U.S. Pat. No. 4,880,838; U.S. Pat. No. 4,920,135; U.S. Pat. No. 5,001,266; U.S. Pat. No. 5,135,953; U.S. Pat. No. 5,166,180; U.S. Pat. No. 5,266,707; U.S. Pat. No. 5,344,842; U.S. Pat. No. 5,424,204; U.S. Pat. No. 5,437,996; U.S. Pat. No. 5,449,812; U.S. Pat. No. 5,589,365; U.S. Pat. No. 5,591,842; U.S. Pat. No. 5,656,642; U.S. Pat. No. 5,668,271; U.S. Pat. No. 5,723,409; U.S. Pat. No. 5,728,699; U.S. Pat. No. 5,760,058; U.S. Pat. No. 5,804,564; U.S. Pat. No. 5,840,917; U.S. Pat. No. 5,849,666; U.S. Pat. No. 5,874,615; U.S. Pat. No. 5,922,740; U.S. Pat. No. 6,060,484; U.S. Pat. No. 6,093,742; U.S. Pat. No. 6,133,258; U.S. Pat. No. 6,136,826; U.S. Pat. No. 6,169,092; U.S. Pat. No. 6,174,905; U.S. Pat. No. 6,207,715; U.S. Pat. No. 6,255,349; U.S. Pat. No. 6,268,387; U.S. Pat. No. 6,335,350; U.S. Pat. No. 6,399,657; U.S. Pat. No. 6,420,396; U.S. Pat. No. 6,541,485; U.S. Pat. No. 6,528,528; U.S. Pat. No. 6,610,715; U.S. Pat. No. 6,677,360; U.S. Pat. No. 6,677,372; U.S. Pat. No. 6,780,873; U.S. Publication No. 2001/0031874; U.S. Publication No. 2002/0016461; U.S. Publication No. 2002/0099210; U.S. Publication No. 2003/0109578; U.S. Publication No. 2003/0109579; U.S. Publication No. 2003/0125318; U.S. Publication No. 2003/0195231; U.S. Publication No. 2004/0009982; U.S. Publication No. 2004/0014754; U.S. Publication No. 2004/0029877; U.S. Publication No. 2004/0030132; U.S. Publication No. 2004/0132727; U.S. Publication No. 2004/0138205; U.S. Publication No. 2004/0147535; U.S. Publication No. 2004/0147569; U.S. Publication No. 2004/0147741; U.S. Publication No. 2004/0162287; CN1183409; DE2303761; EP0518376; EP0728481; JP0603787; JP06287171; JP11335375; JP61106551; JP56025148; WO 1997/003976; WO 1997/011050; WO 1997/030047; WO 1998/042323; WO 1999/059586; WO 2000/035864; WO 2000/076518; WO 2001/021576; WO 2001/047890; WO 2001/047931; WO 2002/089783; WO 2002/090317; WO 2003/037869; WO 2003/097604; WO 2003/097605; WO 2003/099812; WO 2004/013102; WO 2004/020416; or WO 2004/046095.

In another embodiment, a compound of the present invention is a replicase complex defect inducer identified by the method of screening a test compound for replicase complex defect inducer activity comprising: providing a test compound; contacting the test compound with an HCV replicon; and identifying the test compound as an inducer of an HCV replicase complex defect when the level of p14 protein is increased, wherein said replicase complex defect inducer is a compound of Formula (II)

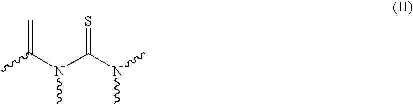

with the proviso that the replicase complex defect inducer is not a compound disclosed in Appendix A, Appendix B, or Appendix C hereto, Baltabaeva et al., Azerbaidzhanskii Kim. Zhur., 4: 97-99 (2000); Baltabaev et al., Khimiko-Farm. Zhur., 36(2): 24-26 (2002); Bloom et al., Biorganic & Med Chem Lett., 13: 2929-2932 (2003); Daugulis et al., Latvijas Kimijas Zurnals, 6 :714-719 (1993); Douglass et al., JACS, 56 (3): 719-721 (1934); Douglass et al., JACS p. 1609 (July 1934); Du et al., Chemistry & Biology, 7(9): 733-742 (2000); Goerdeler et al., Chemische Berichte, 99(11): 3572-3581 (1966); Goerdeler et al., Liebigs Ann Chem 731: 120-141 (1970); Koscik et al., Collect. Czech. Chem. Comm., 48: 3315-3328 (1983); Kulka et al., Canadian J. Chem, 58: 2044-2048 (1980); Kutschy et al., Collect. Czech. Chem. Comm., 64(2): 348-362 (1999); Ludovici et al., Biorganic & Med Chem Lett, 11(17): 2225-2228 (2001); Matosiuk et al., Acta Polonine Pharm. Drug Research, 53(1): 75-77 (1996); Mishra et al., Pharmaceutica Acta Helvetiae, 73: 215-218 (1998); Misra et al., J. fur Praktische Chemie 4 Reihe Band, 36: 256-259 (1967); Mitin et al., Fiziologicheski Aktivnye Veshchestva, 9:31-35 (1977); Mukmeneva et al., Russian J. Applied Chem., 67(4) (1994); Otazo-Sanchez et al., J. Chem. Soc. Perk. Trans., 2: 2211-221 (2001); Patel et al., Indian J. Heter. Chem., 12: 83-84 (2002); Patel et al., Oriental J. Chem, 18(3): 551-554 (2002); Praceus et al., Natruwissenschaften, 51(4): 94-5 (1964); Rashan et al., II Farmaco, 46(5): 677-683 (1991); Rasmussen et al., Synthesis, 6: 456-459 (June 1988); Reynaud et al., Chimie Therapeutique, 7: 421-424 (1966); Schuster, Zentralblatt fur Bakteriologie, Parasitenkunde, Infektionskranheitin un Hygiene Mikrobiolo. Der Landw., 133(7-8): 686-9 (1978); Sengupta et al., Indian J. Chem., 53(1): 203-204 (1976); Seth et al., Tet. Lett., 43: 7303-7306 (2002); Shearer et al., J. Med. Chem., 40(12): 1901-1905 (1997); Taniguchi et al., Chem. Pharm. Bull., 40(1): 240-244 (1992); Weinstein et al., Antibiotics and Chemotherapy, VII(8): 443-448 (1957); Matsuo et al., Chem. Pharm. Bull., 33(10): 4409-4421 (1985); U.S. Pat. No. 3,699,110; U.S. Pat. No. 3,966,968; U.S. Pat. No. 3,931,244; U.S. Pat. No. 4,082,765; U.S. Pat. No. 4,160,037; U.S. Pat. No. 4,338,257; U.S. Pat. No. 4,350,706; U.S. Pat. No. 4,533,676; U.S. Pat. No. 4,540,578; U.S. Pat. No. 4,602,109; U.S. Pat. No. 4,607,044; U.S. Pat. No. 4,638,088; U.S. Pat. No. 4,659,724; U.S. Pat. No. 4,659,736; U.S. Pat. No. 4,665,097; U.S. Pat. No. 4,707,478; U.S. Pat. No. 4,774,260; U.S. Pat. No. 4,868,215; U.S. Pat. No. 4,873,264; U.S. Pat. No. 4,880,838; U.S. Pat. No. 4,920,135; U.S. Pat. No. 5,001,266; U.S. Pat. No. 5,135,953; U.S. Pat. No. 5,166,180; U.S. Pat. No. 5,266,707; U.S. Pat. No. 5,344,842; U.S. Pat. No. 5,424,204; U.S. Pat. No. 5,437,996; U.S. Pat. No. 5,449,812; U.S. Pat. No. 5,589,365; U.S. Pat. No. 5,591,842; U.S. Pat. No. 5,656,642; U.S. Pat. No. 5,668,271; U.S. Pat. No. 5,723,409; U.S. Pat. No. 5,728,699; U.S. Pat. No. 5,760,058; U.S. Pat. No. 5,804,564; U.S. Pat. No. 5,840,917; U.S. Pat. No. 5,849,666; U.S. Pat. No. 5,874,615; U.S. Pat. No. 5,922,740; U.S. Pat. No. 6,060,484; U.S. Pat. No. 6,093,742; U.S. Pat. No. 6,133,258; U.S. Pat. No. 6,136,826; U.S. Pat. No. 6,169,092; U.S. Pat. No. 6,174,905; U.S. Pat. No. 6,207,715; U.S. Pat. No. 6,255,349; U.S. Pat. No. 6,268,387; U.S. Pat. No. 6,335,350; U.S. Pat. No. 6,399,657; U.S. Pat. No. 6,420,396; U.S. Pat. No. 6,541,485; U.S. Pat. No. 6,528,528; U.S. Pat. No. 6,610,715; U.S. Pat. No. 6,677,360; U.S. Pat. No. 6,677,372; U.S. Pat. No. 6,780,873; U.S. Publication No. 2001/0031874; U.S. Publication No. 2002/0016461; U.S. Publication No. 2002/0099210; U.S. Publication No. 2003/0109578; U.S. Publication No. 2003/0109579; U.S. Publication No. 2003/0125318; U.S. Publication No. 2003/0195231; U.S. Publication No. 2004/0009982; U.S. Publication No. 2004/0014754; U.S. Publication No. 2004/0029877; U.S. Publication No. 2004/0030132; U.S. Publication No. 2004/0132727; U.S. Publication No. 2004/0138205; U.S. Publication No. 2004/0147535; U.S. Publication No. 2004/0147569; U.S. Publication No. 2004/0147741; U.S. Publication No. 2004/0162287; CN1183409; DE2303761; EP0518376; EP0728481; JP0603787; JP06287171; JP11335375; JP61106551; JP56025148; WO 1997/003976; WO 1997/011050; WO 1997/030047; WO 1998/042323; WO 1999/059586; WO 2000/035864; WO 2000/076518; WO 2001/021576; WO 2001/047890; WO 2001/047931; WO 2002/089783; WO 2002/090317; WO 2003/037869; WO 2003/097604; WO 2003/097605; WO 2003/099812; WO 2004/013102; WO 2004/020416; or WO 2004/046095.

In another embodiment, a compound of the present invention is a replicase complex defect inducer identified by the method of screening a test compound for replicase complex defect inducer activity comprising: providing a test compound; contacting the test compound with an isolated HCV replicase complex; and identifying the test compound as an inducer of an HCV replicase complex defect when the level of p14 protein is increased, wherein said replicase complex defect inducer is a compound of Formula (II)

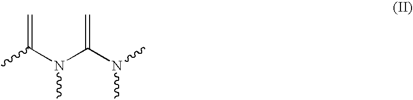

with the proviso that the replicase complex defect inducer is not a compound disclosed in Appendix A, Appendix B, or Appendix C hereto, Baltabaeva et al., Azerbaidzhanskii Kim. Zhur., 4: 97-99 (2000); Baltabaev et al., Khimiko-Farm. Zhur., 36(2): 24-26 (2002); Bloom et al., Biorganic & Med Chem Lett., 13: 2929-2932 (2003); Daugulis et al., Latvijas Kimijas Zurnals, 6 :714-719 (1993); Douglass et al., JACS, 56 (3): 719-721 (1934); Douglass et al., JACS p. 1609 (July 1934); Du et al., Chemistry & Biology, 7(9): 733-742 (2000); Goerdeler et al., Chemische Berichte, 99(11): 3572-3581 (1966); Goerdeler et al., Liebigs Ann Chem 731: 120-141 (1970); Koscik et al., Collect. Czech. Chem. Comm., 48: 3315-3328 (1983); Kulka et al., Canadian J. Chem, 58: 2044-2048 (1980); Kutschy et al., Collect. Czech. Chem. Comm., 64(2): 348-362 (1999); Ludovici et al., Biorganic & Med Chem Lett, 11(17): 2225-2228 (2001); Matosiuk et al., Acta Polonine Pharm. Drug Research, 53(1): 75-77 (1996); Mishra et al., PharmaceuticaActa Helvetiae, 73: 215-218 (1998); Misra et al., J. fur Praktische Chemie 4 Reihe Band, 36: 256-259 (1967); Mitin et al., Fiziologicheski Aktivnye Veshchestva, 9:31-35 (1977); Mukmeneva et al., Russian J. Applied Chem., 67(4) (1994); Otazo-Sanchez et al., J. Chem. Soc. Perk. Trans., 2: 2211-221 (2001); Patel et al., Indian J. Heter. Chem., 12: 83-84 (2002); Patel et al., Oriental J. Chem, 18(3): 551-554 (2002); Praceus et al., Natruwissenschaften, 51(4): 94-5 (1964); Rashan et al., II Farmaco, 46(5): 677-683 (1991); Rasmussen et al., Synthesis, 6: 456-459 (June 1988); Reynaud et al., Chimie Therapeutique, 7: 421-424 (1966); Schuster, Zentralblatt fur Bakteriologie, Parasitenkunde, Infektionskranheitin un Hygiene Mikrobiolo. Der Landw., 133(7-8): 686-9 (1978); Sengupta et al., Indian J. Chem., 53(1): 203-204 (1976); Seth et al., Tet. Lett., 43: 7303-7306 (2002); Shearer et al., J. Med. Chem., 40(12): 1901-1905 (1997); Taniguchi et al., Chem. Pharm. Bull., 40(1): 240-244 (1992); Weinstein et al., Antibiotics and Chemotherapy, VII(8): 443-448 (1957); Matsuo et al., Chem. Pharm. Bull., 33(10): 4409-4421 (1985); U.S. Pat. No. 3,699,110; U.S. Pat. No. 3,966,968; U.S. Pat. No. 3,931,244; U.S. Pat. No. 4,082,765; U.S. Pat. No. 4,160,037; U.S. Pat. No. 4,338,257; U.S. Pat. No. 4,350,706; U.S. Pat. No. 4,533,676; U.S. Pat. No. 4,540,578; U.S. Pat. No. 4,602,109; U.S. Pat. No. 4,607,044; U.S. Pat. No. 4,638,088; U.S. Pat. No. 4,659,724; U.S. Pat. No. 4,659,736; U.S. Pat. No. 4,665,097; U.S. Pat. No. 4,707,478; U.S. Pat. No. 4,774,260; U.S. Pat. No. 4,868,215; U.S. Pat. No. 4,873,264; U.S. Pat. No. 4,880,838; U.S. Pat. No. 4,920,135; U.S. Pat. No. 5,001,266; U.S. Pat. No. 5,135,953; U.S. Pat. No. 5,166,180; U.S. Pat. No. 5,266,707; U.S. Pat. No. 5,344,842; U.S. Pat. No. 5,424,204; U.S. Pat. No. 5,437,996; U.S. Pat. No. 5,449,812; U.S. Pat. No. 5,589,365; U.S. Pat. No. 5,591,842; U.S. Pat. No. 5,656,642; U.S. Pat. No. 5,668,271; U.S. Pat. No. 5,723,409; U.S. Pat. No. 5,728,699; U.S. Pat. No. 5,760,058; U.S. Pat. No. 5,804,564; U.S. Pat. No. 5,840,917; U.S. Pat. No. 5,849,666; U.S. Pat. No. 5,874,615; U.S. Pat. No. 5,922,740; U.S. Pat. No. 6,060,484; U.S. Pat. No. 6,093,742; U.S. Pat. No. 6,133,258; U.S. Pat. No. 6,136,826; U.S. Pat. No. 6,169,092; U.S. Pat. No. 6,174,905; U.S. Pat. No. 6,207,715; U.S. Pat. No. 6,255,349; U.S. Pat. No. 6,268,387; U.S. Pat. No. 6,335,350; U.S. Pat. No. 6,399,657; U.S. Pat. No. 6,420,396; U.S. Pat. No. 6,541,485; U.S. Pat. No. 6,528,528; U.S. Pat. No. 6,610,715; U.S. Pat. No. 6,677,360; U.S. Pat. No. 6,677,372; U.S. Pat. No. 6,780,873; U.S. Publication No. 2001/0031874; U.S. Publication No. 2002/0016461; U.S. Publication No. 2002/0099210; U.S. Publication No. 2003/0109578; U.S. Publication No. 2003/0109579; U.S. Publication No. 2003/0125318; U.S. Publication No. 2003/0195231; U.S. Publication No. 2004/0009982; U.S. Publication No. 2004/0014754; U.S. Publication No. 2004/0029877; U.S. Publication No. 2004/0030132; U.S. Publication No. 2004/0132727; U.S. Publication No. 2004/0138205; U.S. Publication No. 2004/0147535; U.S. Publication No. 2004/0147569; U.S. Publication No. 2004/0147741; U.S. Publication No. 2004/0162287; CN1183409; DE2303761; EP0518376; EP0728481; JP0603787; JP06287171; JP11335375; JP61106551; JP56025148; WO 1997/003976; WO 1997/011050; WO 1997/030047; WO 1998/042323; WO 1999/059586; WO 2000/035864; WO 2000/076518; WO 2001/021576; WO 2001/047890; WO 2001/047931; WO 2002/089783; WO 2002/090317; WO 2003/037869; WO 2003/097604; WO 2003/097605; WO 2003/099812; WO 2004/013102; WO 2004/020416; or WO 2004/046095.

In another embodiment, a compound of the present invention is a replicase complex defect inducer identified by the method of screening a test compound for replicase complex defect inducer activity comprising: providing a test compound; contacting the test compound with an isolated HCV polyprotein or fragment thereof; and identifying the test compound as an inducer of an HCV replicase complex defect when the level of p14 protein is increased, wherein said replicase complex defect inducer is a compound of Formula (II)

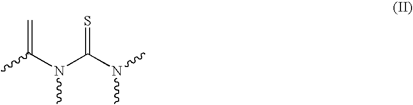

with the proviso that the replicase complex defect inducer is not a compound disclosed in Appendix A, Appendix B, or Appendix C hereto, Baltabaeva et al., Azerbaidzhanskii Kim. Zhur., 4: 97-99 (2000); Baltabaev et al., Khimiko-Farm. Zhur., 36(2): 24-26 (2002); Bloom et al., Biorganic & Med Chem Lett., 13: 2929-2932 (2003); Daugulis et al., Latvijas Kimijas Zurnals, 6 :714-719 (1993); Douglass et al., JACS, 56 (3): 719-721 (1934); Douglass et al., JACS p. 1609 (July 1934); Du et al., Chemistry & Biology, 7(9): 733-742 (2000); Goerdeler et al., Chemische Berichte, 99(11): 3572-3581 (1966); Goerdeler et al., Liebigs Ann Chem 731: 120-141 (1970); Koscik et al., Collect. Czech. Chem. Comm., 48: 3315-3328 (1983); Kulka et al., Canadian J. Chem, 58: 2044-2048 (1980); Kutschy et al., Collect. Czech. Chem. Comm., 64(2): 348-362 (1999); Ludovici et al., Biorganic & Med Chem Lett, 11(17): 2225-2228 (2001); Matosiuk et al., Acta Polonine Pharm. Drug Research, 53(1): 75-77 (1996); Mishra et al., Pharmaceutica Acta Helvetiae, 73: 215-218 (1998); Misra et al., J. fur Praktische Chemie 4 Reihe Band, 36: 256-259 (1967); Mitin et al., Fiziologicheski Aktivnye Veshchestva, 9:31-35 (1977); Mukmeneva et al., Russian J. Applied Chem., 67(4) (1994); Otazo-Sanchez et al., J. Chem. Soc. Perk. Trans., 2: 2211-221 (2001); Patel et al., Indian J. Heter. Chem., 12: 83-84 (2002); Patel et al., Oriental J. Chem, 18(3): 551-554 (2002); Praceus et al., Natruwissenschaften, 51(4): 94-5 (1964); Rashan et al., II Farmaco, 46(5): 677-683 (1991); Rasmussen et al., Synthesis, 6: 456-459 (June 1988); Reynaud et al., Chimie Therapeutique, 7: 421-424 (1966); Schuster, Zentralblatt fur Bakteriologie, Parasitenkunde, Infektionskranheitin un Hygiene Mikrobiolo. Der Landw., 133(7-8): 686-9 (1978); Sengupta et al., Indian J. Chem., 53(1): 203-204 (1976); Seth et al., Tet. Lett., 43: 7303-7306 (2002); Shearer et al., J. Med. Chem., 40(12): 1901-1905 (1997); Taniguchi et al., Chem. Pharm. Bull., 40(1): 240-244 (1992); Weinstein et al., Antibiotics and Chemotherapy, VII(8): 443-448 (1957); Matsuo et al., Chem. Pharm. Bull., 33(10): 4409-4421 (1985); U.S. Pat. No. 3,699,110; U.S. Pat. No. 3,966,968; U.S. Pat. No. 3,931,244; U.S. Pat. No. 4,082,765; U.S. Pat. No. 4,160,037; U.S. Pat. No. 4,338,257; U.S. Pat. No. 4,350,706; U.S. Pat. No. 4,533,676; U.S. Pat. No. 4,540,578; U.S. Pat. No. 4,602,109; U.S. Pat. No. 4,607,044; U.S. Pat. No. 4,638,088; U.S. Pat. No. 4,659,724; U.S. Pat. No. 4,659,736; U.S. Pat. No. 4,665,097; U.S. Pat. No. 4,707,478; U.S. Pat. No. 4,774,260; U.S. Pat. No. 4,868,215; U.S. Pat. No. 4,873,264; U.S. Pat. No. 4,880,838; U.S. Pat. No. 4,920,135; U.S. Pat. No. 5,001,266; U.S. Pat. No. 5,135,953; U.S. Pat. No. 5,166,180; U.S. Pat. No. 5,266,707; U.S. Pat. No. 5,344,842; U.S. Pat. No. 5,424,204; U.S. Pat. No. 5,437,996; U.S. Pat. No. 5,449,812; U.S. Pat. No. 5,589,365; U.S. Pat. No. 5,591,842; U.S. Pat. No. 5,656,642; U.S. Pat. No. 5,668,271; U.S. Pat. No. 5,723,409; U.S. Pat. No. 5,728,699; U.S. Pat. No. 5,760,058; U.S. Pat. No. 5,804,564; U.S. Pat. No. 5,840,917; U.S. Pat. No. 5,849,666; U.S. Pat. No. 5,874,615; U.S. Pat. No. 5,922,740; U.S. Pat. No. 6,060,484; U.S. Pat. No. 6,093,742; U.S. Pat. No. 6,133,258; U.S. Pat. No. 6,136,826; U.S. Pat. No. 6,169,092; U.S. Pat. No. 6,174,905; U.S. Pat. No. 6,207,715; U.S. Pat. No. 6,255,349; U.S. Pat. No. 6,268,387; U.S. Pat. No. 6,335,350; U.S. Pat. No. 6,399,657; U.S. Pat. No. 6,420,396; U.S. Pat. No. 6,541,485; U.S. Pat. No. 6,528,528; U.S. Pat. No. 6,610,715; U.S. Pat. No. 6,677,360; U.S. Pat. No. 6,677,372; U.S. Pat. No. 6,780,873; U.S. Publication No. 2001/0031874; U.S. Publication No. 2002/0016461; U.S. Publication No. 2002/0099210; U.S. Publication No. 2003/0109578; U.S. Publication No. 2003/0109579; U.S. Publication No. 2003/0125318; U.S. Publication No. 2003/0195231; U.S. Publication No. 2004/0009982; U.S. Publication No. 2004/0014754; U.S. Publication No. 2004/0029877; U.S. Publication No. 2004/0030132; U.S. Publication No. 2004/0132727; U.S. Publication No. 2004/0138205; U.S. Publication No. 2004/0147535; U.S. Publication No. 2004/0147569; U.S. Publication No. 2004/0147741; U.S. Publication No. 2004/0162287; CN1183409; DE2303761; EP0518376; EP0728481; JP0603787; JP06287171; JP11335375; JP61106551; JP56025148; WO 1997/003976; WO 1997/011050; WO 1997/030047; WO 1998/042323; WO 1999/059586; WO 2000/035864; WO 2000/076518; WO 2001/021576; WO 2001/047890; WO 2001/047931; WO 2002/089783; WO 2002/090317; WO 2003/037869; WO 2003/097604; WO 2003/097605; WO 2003/099812; WO 2004/013102; WO 2004/020416; or WO 2004/046095.

A compound of the present invention includes a compound identified by any of the methods of the present invention. In an embodiment, a compound of the present invention is a replicase complex defect inducer identified by any of the methods of the present invention, wherein said replicase complex defect inducer is a compound of Formula (III)

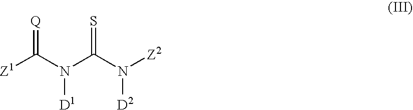

wherein
Q is oxygen or sulfur; and
D¹ and D² are independently selected from the group consisting of hydrogen and methyl.

In the context of the present invention, Z¹ and Z² may include any independently selected substituents, including any optionally substituted substituents.

In an embodiment, a compound of the present invention is a replicase complex defect inducer identified by any of the methods of the present invention, wherein said replicase complex defect inducer is a compound of Formula (III)

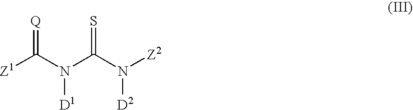

wherein
Q is oxygen or sulfur;
D¹ and D² are independently selected from the group consisting of hydrogen and methyl;
and further wherein the compound is a compound of Formula (I)

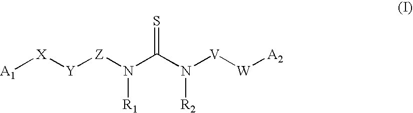

wherein

A₁ and A₂ are independently optionally substituted C₁-C₁₂ alkyl, optionally substituted mono- or di-(C₁-C₈ alkyl)amino, optionally substituted C₂-C₁₂ alkenyl, optionally substituted C₃-C₈ cycloalkyl, a partially unsaturated or aromatic carbocyclic group, or an optionally substituted saturated, partially unsaturated, or aromatic heterocyclic group; wherein at least one of A₁ and A₂ is an optionally substituted aromatic carbocyclic group or an optionally substituted aromatic heterocyclic group.

X and W are independently O, S, NR, or absent, where R is hydrogen, optionally substituted C₁-C₆ alkyl, or optionally substituted aryl(C₀-C₄ alkyl).

V is C₁-C₆ alkyl, C₂-C₆ alkenyl, C₃-C₇ cycloalkyl, or absent; and Y is C₁-C₆ alkyl, C₁-C₆ alkyl substituted with C₃-C₇ cycloalkyl, C₂-C₆ alkenyl, C₃-C₇ cycloalkyl, or absent; wherein when V is absent, W is absent; and Z is carbonyl, thiocarbonyl, imino, or C₁-C₆ alkylimino.

R₁ and R₂ are independently hydrogen or R₁ and R₂ are independently C₁-C₆ alkyl, C₂-C₆ alkenyl, or C₂-C₆ alkynyl, each of which is substituted with 0 to 3 substituents independently chosen from halogen, hydroxy, amino, C₁-C₄ alkoxy, C₁-C₂ haloalkyl, and C₁-C₂ haloalkoxy, or R₁ and R₂ are joined to form a 5- to 7-membered saturated or mono-unsaturated ring optionally containing one additional heteroatom chosen from N, S, and O, which 5- to 7-membered saturated or mono-unsaturated ring is substituted with 0 to 3 substituents independently chosen from halogen, hydroxy, amino, C₁-C₄ alkyl, C₁-C₄ alkoxy, mono- and di-(C₁-C₄alkyl)amino, C₁-C₂ haloalkyl, and C₁-C₂ haloalkoxy.

In an embodiment, a compound of the present invention is a replicase complex defect inducer identified by the method of screening a test compound for replicase complex defect inducer activity comprising: providing a test compound; contacting the test compound with a cell expressing an HCV virion that comprises an NS3 protein with a mutation at or within about 15 angstroms of C16; and identifying the test compound as an inducer of an HCV replicase complex defect when the cell or HCV virion is resistant to the test compound, wherein said compound is a compound of Formula (III)

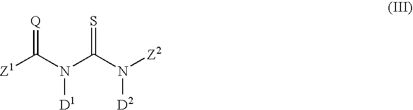

wherein
Q is oxygen or sulfur;
D¹ and D² are independently selected from the group consisting of hydrogen and methyl;
with the proviso that said compound of Formula (III) is not a compound of the formula (III-a):

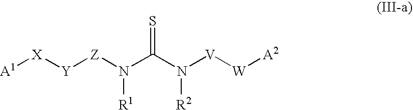

wherein

A₁ and A₂ are independently optionally substituted C₁-C₁₂ alkyl, optionally substituted mono- or di-(C₁-C₈ alkyl)amino, optionally substituted C₂-C₁₂ alkenyl, optionally substituted C₃-C₈ cycloalkyl, an optionally substituted partially unsaturated or aromatic carbocyclic group, or an optionally substituted saturated, partially unsaturated, or aromatic heterocyclic group, wherein at least one of A₁ and A₂ is an optionally substituted aromatic carbocyclic group or an optionally substituted aromatic heterocyclic group;

X and W are independently O, S, NR, or absent, wherein R is hydrogen, optionally substituted (C₁-C₆)alkyl, or optionally substituted aryl(C₀-C₄)alkyl;

V is (C₁-C₆)alkyl, (C₂-C₆)alkenyl, (C₃-C₇)cycloalkyl, or absent, wherein when V is absent, W is absent; and

Y is (C₁-C₆) alkyl, (C₁-C₆)alkyl substituted with (C₃-C₇)cycloalkyl, (C₂-C₆) alkenyl, (C₃-C₇)cycloalkyl, or absent; and

Z is carbonyl, thiocarbonyl, imino, or C₁-C₆ alkylimino; and

R₁ and R₂ are independently hydrogen or methyl;

and with the further proviso that said compound of Formula (III) is not a compound of the formula (III-b):

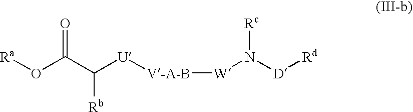

wherein
R^{a} is hydrogen, a substituted or unsubstituted alkyl or cycloalkyl group, a substituted or unsubstituted aryl group or a saturated or unsaturated, optionally substituted heterocyclic group;
R^{b} is hydrogen, a substituted or unsubstituted alkyl or cycloalkyl group, a substituted or unsubstituted aryl group, a saturated or unsaturated, optionally substituted heterocyclic group, an optionally substituted alkenyl group, an optionally substituted alkinyl group, —NR^{2′}SO₂R^{2″}, —NR^{2′}COOR^{2′}, —NR^{2′}COR^{2′}, —NR^{2′}CN(R^{2′})₂, or —N R^{2′}CSN(R^{2′})₂;
R^{2′} is hydrogen, a substituted or unsubstituted alkyl or cycloalkyl group, a substituted or unsubstituted aryl group or a saturated or unsaturated, optionally substituted heterocyclic group;
R^{2″} is a substituted or unsubstituted alkyl, alkenyl or cycloalkyl group, a substituted or unsubstituted aryl group, or a saturated or unsaturated, optionally substituted heterocyclic group;
U′ is a direct bond or a substituted or unsubstituted alkylene group;
V′ is a substituted or unsubstituted alkylene group, —NR^{2′}CO—, or —NR^{2′}SO₂—;
A and B are each independently an optionally substituted 1,3- or 1,4-bridging phenylene group or an optionally substituted 2,4- or 2,5-bridging thienylene group;
W′ is a direct bond or an optionally substituted alkylene group;
D′ is a direct bond or

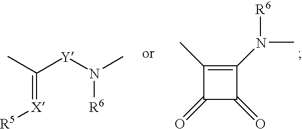

R^{c} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{d}, Y′, R⁵, or R⁶, if present, with formation of an optionally substituted heterocyclic ring system which includes the nitrogen atom to which R^{c} is bonded, and may be saturated or unsaturated and may contain further heteroatoms;
R^{d} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{c}, Y, R⁵ or R⁶, if present, with formation of an optionally substituted heterocyclic ring system which includes the nitrogen atom to which R^{d} is bonded and may be saturated or unsaturated and may contain further heteroatoms;

### X′ is CHNO₂, CHCN, O, N or S;

Y′ is a direct bond or an optionally substituted alkylene or alkine group;
R⁵ is absent, or is hydrogen, a substituted or unsubstituted alkyl or cycloalkyl group, —NO₂, —CN, —COR^{5′}, —COOR^{5′}, or is connected to one of R^{c}, Y′, R^{d}, or R⁶, if present, with formation of an optionally substituted carbocyclic or heterocyclic ring system which includes X′ and can be saturated or unsaturated and may optionally contain one or more additional heteroatoms;
R^{5′} is hydrogen, a substituted or unsubstituted alkyl or cycloalkyl group, a substituted or unsubstituted aryl group or a saturated or unsaturated, optionally substituted heterocyclic group which may optionally contain one or more additional heteroatoms;
R⁶ is a arylcarbonyl group, or a heteroarylcarbonyl group;
and wherein if A is a phenylene group and V′ is —NR^{2′}CO— or —NR^{2′} SO₂—, D′ is not a direct bond and X′ is not N;
and with the further proviso that said compound of Formula (III) is not a compound of the formula (III-c)

X^{1″}—Y^{1″}-Z^{1″}-W^{1″} (III-c)

wherein
X^{1″} is optionally substituted aryl, optionally substituted (C₁-C₈)alkyl-aryl, optionally substituted heteroaryl, or optionally substituted heteroarylalkyl;
Y^{1″} is —NR^{A}CSNR^{A}CO—, wherein R^{A} is independently hydrogen or lower alkyl;
Z^{1″} is optionally substituted phenylene, optionally substituted monocyclic heteroarylene, optionally substituted monocyclic non-aromatic heterocycle-diyl, or optionally substituted monocyclic cycloalkane-diyl;
W^{1″} is a group represented by the formula:

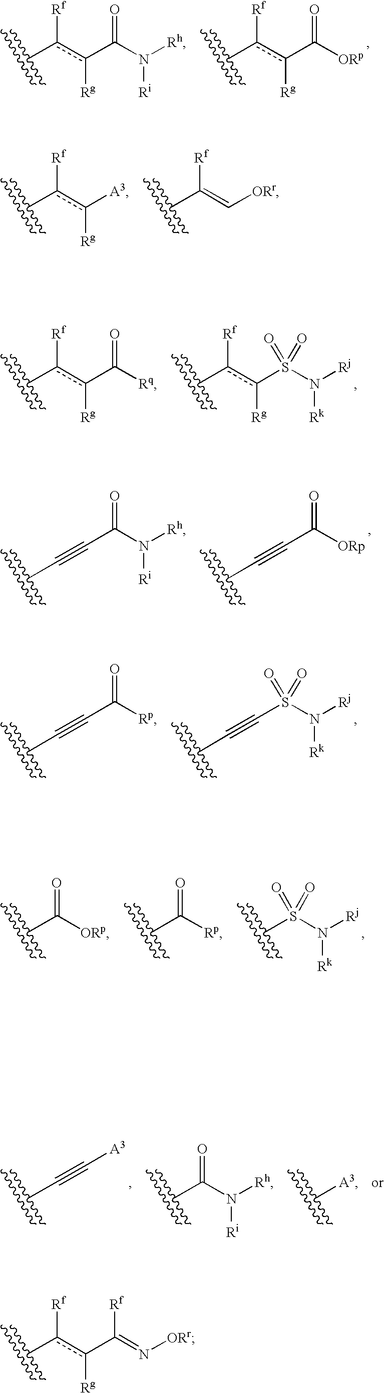

wherein
R^{f}, R^{g}, R^{h}, Rⁱ, R^{j}, and R^{k} are each independently hydrogen, halogen, optionally substituted lower alkyl, optionally substituted lower alkyloxy, optionally substituted lower alkylthio, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, optionally substituted non-aromatic heterocyclic group, or optionally substituted amino;
R^{p}, R^{q}, and R^{r} are each independently hydrogen, optionally substituted lower alkyl, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, or optionally substituted non-aromatic heterocyclic group;
A³ is an optionally substituted aryl or an optionally substituted heteroaryl group;
and with the further proviso that in said compound of Formula (III), Z¹ is not a 3-pyridyl group or a substituted 3-pyridyl group when Z² is —OH, —NH₂, —(C₁-C₆)alkylamino, di(C₁-C₆)alkylamino, an alkyl group, a substituted alkyl group, —(C₃-C₈) cycloalkyl group, a substituted —(C₃-C₈) cycloalkyl group, —(C₃-C₈) cycloalkyl-(C₁-C₆)alkyl group, —O-alkyl group, a substituted —O-alkyl group, —O—(C₃-C₈) cycloalkyl group, a substituted —O—(C₃-C₈) cycloalkyl group, a phenyl group, a fused-phenyl group, a phenyl group substituted with one or more independently selected halogen, —OCH₃, nitro, or dimethyl amino groups, a substituted fused-phenyl group substituted with one or more independently selected halogen, —OCH₃, nitro, or dimethyl amino groups, a —(C₁-C₈) alkyl-phenyl group, —O—(C₀-C₈) alkyl-phenyl group, —(C₃-C₆) alkylene group, —O—(C₃-C₆) alkylene group, —(C₃-C₆) alkynyl group, —O—(C₃-C₆) alkynyl group, —CO(C₁-C₆) alkyl group, —NHCO(C₁-C₆) alkyl group, —NHSO₂(C₁-C₆) alkyl group, —SO₂(C₁-C₆) alkyl group, or —SO₂(C₀-C₆)alkyl-phenyl group;
and with the further proviso that in said compound of Formula (III), Z¹ is not a furyl group or a substituted furyl group when D² and Z² are independently selected from the group consisting of hydrogen and methyl;
and with the further proviso that in said compound comprising structure (III), when D² is —CH₃ and Z² is an optionally substituted phenyl, Z¹ is not selected from the group consisting of —R³³, OR³⁴, and —NR³⁵R³⁶,
wherein
R³³ is hydrogen, optionally substituted (C₃-C₈)cycloalkyl, optionally substituted phenyl, optionally substituted heteroaryl or phenyl-(C₁-C₄) alkyl which is optionally substituted on the phenyl ring, (C₁-C₆)alkyl, (C₂-C₆)alkenyl, (C₂-C₆)alkynyl, the three latter radicals independently being optionally substituted with one or more radicals selected from the group consisting of halogen, (C₁-C₄)alkoxy, (C₁-C₄)alkylthio and NR³⁷R³⁸;
R³⁴ is (C₁-C₆)alkyl, (C₂-C₆)alkenyl, (C₂-C₆)alkynyl, the three latter radicals independently optionally substituted with one or more radicals selected from the group consisting of halogen, (C₁-C₄)alkoxy, (C₁-C₄)alkylthio and NR³⁷R³⁸, or R³⁴ is (C₃-C₆)cycloalkyl optionally substituted with one or more groups selected from the group consisting of halogen, (C₁-C₄)alkyl and (C₁-C₄)alkoxy, and (C₃-C₆)-cycloalkyl-(C₁-C₃)alkyl;
R³⁵ and R³⁶ are hydrogen, (C₁-C₆)alkyl, (C₂-C₆)alkenyl, (C₂-C₆)alkynyl, the 3 latter groups independently optionally substituted by one or more halogen radicals;
R³⁷ is independently selected from the group consisting of hydrogen, (C₁-C₄)alkyl, ((C₁-C₄)alkyl)carbonyl, ((C₁-C₄)alkoxy)carbonyl and CHO;
R³⁸ is independently selected from the group consisting of H and (C₁-C₄)alkyl;
and with the further proviso that in said compound of Formula (III), Z¹ and Z² are not both selected from the group consisting of hydrogen and alkyl;
and with the further proviso that in said compound of Formula (III), Z² is not hydrogen when D¹ and D² are both hydrogen;
and with the further proviso that in said compound of Formula (III), Z¹ is not (C₁-C₆) alkyl, (C₁-C₆) alkoxy, pyridyl, or aryl when D¹ and D² are both hydrogen and when Z² is selected from the group consisting of hydrogen, a (C₁-C₆) alkyl group optionally substituted with 1 to 3 substituents independently selected from the group consisting of hydroxyl, (C₁-C₆) alkoxyl, carboxyl, (C₁-C₆) alkoxycarbonyl, lower alkylthio, fluorine, chlorine, bromine, iodine, amino, mono- or di-substituted amino, phthalimido and nitrogen-containing heterocyclic groups, a (C₃-C₈) cycloalkyl group, a (C₂-C₆) alkenyl group, an arylalkyl group optionally substituted with from 1 to 5 substituents independently selected from the group consisting of (C₁-C₆) alkyl, hydroxyl, (C₁-C₆) alkoxy, halogen, nitro, and amino groups, an aryl group optionally substituted with from 1 to 5 independently selected substituents selected from the group consisting of (C₁-C₆) alkyl, hydroxyl, (C₁-C₆) alkoxy, halogen, nitro, and amino groups, a substituted or unsubstituted heterocyclic group, a (C₁-C₆) alkanoyl group, a benzoyl or a naphthoyl group either of which is optionally substituted with from 1 to 5 independently selected substituents selected from the group consisting of (C₁-C₆) alkyl, hydroxyl, (C₁-C₆) alkoxy, halogen nitro, and amino groups, a pyridylcarbonyl group, a (C₁-C₆) alkoxycarbonyl group or an amino group optionally substituted with (C₁-C₆) alkyl, arylalkyl having 7 to 15 carbon atoms, substituted or unsubstituted aryl, (C₁-C₆) alkanoyl, optionally substituted aroyl, and (C₁-C₆) alkylidene groups;
and with the further proviso that in said compound of Formula (III), Z¹ is not —O—(CH₂)ₜ—CH₃, wherein t is 0-4 and Z² is halophenyl;
and with the further proviso that in said compound of Formula (III), Z¹ is not a substituted pyridyl, pyrimidinyl, pyrazinyl, pyridazinyl group substituted with —N(R²¹)-(optionally substituted phenyl), wherein said R²¹ is hydrogen, aryl, formyl, (C₁-C₆)alkylcarbonyl, (C₁-C₆)alkyl, (C₁-C₆)alkyloxycarbonyl, (C₁-C₆)alkyl substituted with formyl, (C₁-C₆)alkylcarbonyl, (C₁-C₆)alkyloxycarbonyl, (C₁-C₆)alkylcarbonyloxy, or (C₁-C₆)alkyloxy(C₁-C₆)alkylcarbonyl optionally substituted with (C₁-C₆)alkyloxycarbonyl;
and with the further proviso that in said compound of Formula (III), Z¹ is not a 3-(4-trifluoromethyl-pyridinyl) group or a 3-(4-trifluoromethyl-pyridinyl N-oxide) group;
and with the further proviso that in said compound of Formula (III), Z¹ is not a substituted or unsubstituted group selected from the group consisting of

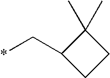

and a —(CH₂)ₜ-adamantanyl group, wherein t is 0-4;
and with the further proviso that in said compound of Formula (III), Z² is not a

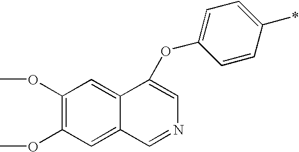

group, which is unsubstituted, or which is substituted with one or more substituents independently selected from the group consisting of halogens and alkyl groups;
and with the further proviso that in said compound of Formula (III), Z² is not a substituted or unsubstituted group selected from the group consisting of:

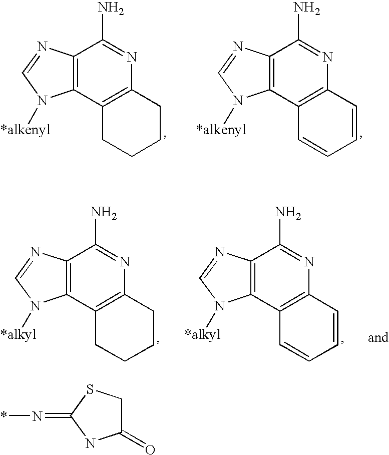

and with the further proviso that said compound of Formula (III), is not a compound set forth in Appendix A, Appendix B, or Appendix C.

In an embodiment, a compound of the present invention is a replicase complex defect inducer identified by the method of screening a test compound for replicase complex defect inducer activity comprising: providing a test compound; contacting the test compound with a cell expressing an HCV replicon that comprises an NS3 protein with a mutation at or within about 15 angstroms of C16; and identifying the test compound as an inducer of an HCV replicase complex defect when the cell or HCV replicon is resistant to the test compound, wherein said compound is a compound of Formula (II)

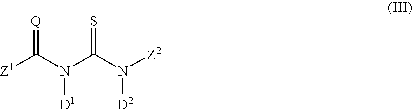

wherein
Q is oxygen or sulfur;
D¹ and D² are independently selected from the group consisting of hydrogen and methyl;
with the proviso that said compound of Formula (III) is not a compound of the formula (III-a):

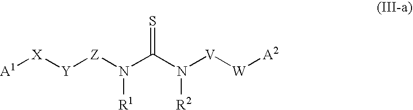

wherein

A₁ and A₂ are independently optionally substituted C₁-C₁₂ alkyl, optionally substituted mono- or di-(C₁-C₈ alkyl)amino, optionally substituted C₂-C₁₂ alkenyl, optionally substituted C₃-C₈ cycloalkyl, an optionally substituted partially unsaturated or aromatic carbocyclic group, or an optionally substituted saturated, partially unsaturated, or aromatic heterocyclic group, wherein at least one of A₁ and A₂ is an optionally substituted aromatic carbocyclic group or an optionally substituted aromatic heterocyclic group;

X and W are independently O, S, NR, or absent, wherein R is hydrogen, optionally substituted (C₁-C₆)alkyl, or optionally substituted aryl(C₀-C₄)alkyl;

V is (C₁-C₆)alkyl, (C₂-C₆)alkenyl, (C₃-C₇)cycloalkyl, or absent, wherein when V is absent, W is absent; and

Y is (C₁-C₆) alkyl, (C₁-C₆)alkyl substituted with (C₃-C₇)cycloalkyl, (C₂-C₆) alkenyl, (C₃-C₇)cycloalkyl, or absent; and

Z is carbonyl, thiocarbonyl, imino, or C₁-C₆ alkylimino; and

R₁ and R₂ are independently hydrogen or methyl;

and with the further proviso that said compound of Formula (III) is not a compound of the formula (III-b):

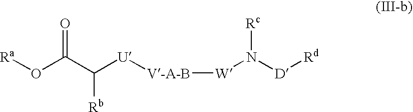

wherein
R^{a} is hydrogen, a substituted or unsubstituted alkyl or cycloalkyl group, a substituted or unsubstituted aryl group or a saturated or unsaturated, optionally substituted heterocyclic group;
R^{b} is hydrogen, a substituted or unsubstituted alkyl or cycloalkyl group, a substituted or unsubstituted aryl group, a saturated or unsaturated, optionally substituted heterocyclic group, an optionally substituted alkenyl group, an optionally substituted alkinyl group, —NR^{2′}SO₂R^{2″}, —NR^{2′}COOR^{2′}, —NR^{2′}COR^{2′}, —NR^{2′}CON(R^{2′})₂, or —N R^{2′}CSN(R^{2′})₂;
R^{2′} is hydrogen, a substituted or unsubstituted alkyl or cycloalkyl group, a substituted or unsubstituted aryl group or a saturated or unsaturated, optionally substituted heterocyclic group;
R^{2″} is a substituted or unsubstituted alkyl, alkenyl or cycloalkyl group, a substituted or unsubstituted aryl group, or a saturated or unsaturated, optionally substituted heterocyclic group;
U′ is a direct bond or a substituted or unsubstituted alkylene group;
V′ is a substituted or unsubstituted alkylene group, —NR^{2′}CO—, or —NR^{2′}SO₂—;
A and B are each independently an optionally substituted 1,3- or 1,4-bridging phenylene group or an optionally substituted 2,4- or 2,5-bridging thienylene group;
W′ is a direct bond or an optionally substituted alkylene group;
D′ is a direct bond or

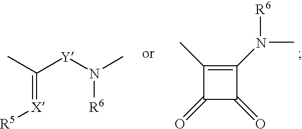

R^{c} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{d}, Y′, R⁵, or R⁶, if present, with formation of an optionally substituted heterocyclic ring system which includes the nitrogen atom to which R^{c} is bonded, and may be saturated or unsaturated and may contain further heteroatoms;
R^{d} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{c}, Y, R⁵ or R⁶, if present, with formation of an optionally substituted heterocyclic ring system which includes the nitrogen atom to which R^{d} is bonded and may be saturated or unsaturated and may contain further heteroatoms;

### X′ is CHNO₂, CHCN, O, N or S;

Y′ is a direct bond or an optionally substituted alkylene or alkine group;
R⁵ is absent, or is hydrogen, a substituted or unsubstituted alkyl or cycloalkyl group, —NO₂, —CN, —COR^{5′}, —COOR^{5′}, or is connected to one of R^{c}, Y′, R^{d}, or R⁶, if present, with formation of an optionally substituted carbocyclic or heterocyclic ring system which includes X′ and can be saturated or unsaturated and may optionally contain one or more additional heteroatoms;
R^{5′} is hydrogen, a substituted or unsubstituted alkyl or cycloalkyl group, a substituted or unsubstituted aryl group or a saturated or unsaturated, optionally substituted heterocyclic group which may optionally contain one or more additional heteroatoms;
R⁶ is a arylcarbonyl group, or a heteroarylcarbonyl group;
and wherein if A is a phenylene group and V′ is —NR^{2′}CO— or —NR^{2′} SO₂—, D′ is not a direct bond and X′ is not N;
and with the further proviso that said compound of Formula (III) is not a compound of the formula (III-c)

X^{1″}—Y^{1″}-Z^{1″}-W^{1″} (III-c)

wherein
X^{1″} is optionally substituted aryl, optionally substituted (C₁-C₈)alkyl-aryl, optionally substituted heteroaryl, or optionally substituted heteroarylalkyl;
Y^{1″} is —NR^{A}CSNR^{A}CO—, wherein R^{A} is independently hydrogen or lower alkyl;
Z^{1″} is optionally substituted phenylene, optionally substituted monocyclic heteroarylene, optionally substituted monocyclic non-aromatic heterocycle-diyl, or optionally substituted monocyclic cycloalkane-diyl;
W^{1″} is a group represented by the formula:

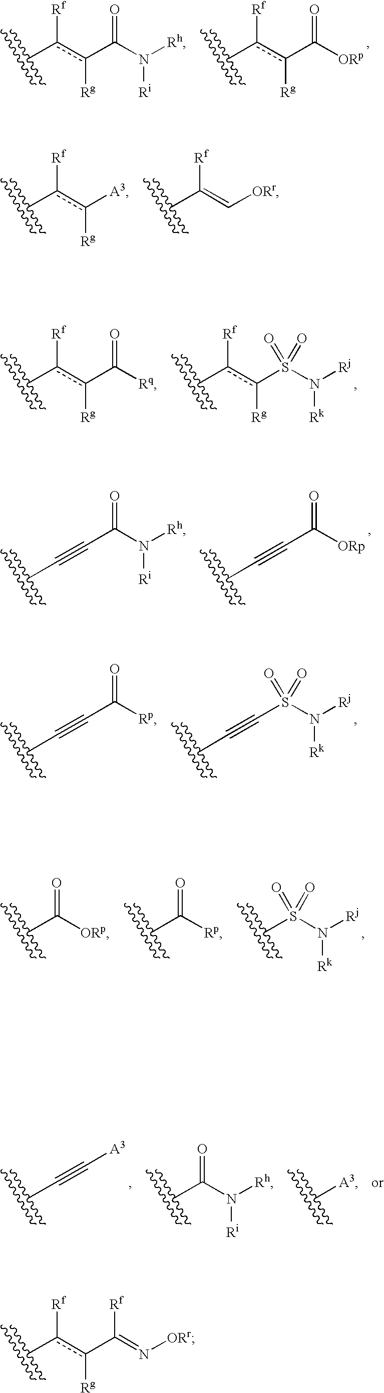

wherein
R^{f}, R^{g}, R^{h}, Rⁱ, R^{j}, and R^{k} are each independently hydrogen, halogen, optionally substituted lower alkyl, optionally substituted lower alkyloxy, optionally substituted lower alkylthio, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, optionally substituted non-aromatic heterocyclic group, or optionally substituted amino;
R^{p}, R^{q}, and R^{r} are each independently hydrogen, optionally substituted lower alkyl, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, or optionally substituted non-aromatic heterocyclic group;
A³ is an optionally substituted aryl or an optionally substituted heteroaryl group;
and with the further proviso that in said compound of Formula (III), Z¹ is not a 3-pyridyl group or a substituted 3-pyridyl group when Z² is —OH, —NH₂, —(C₁-C₆)alkylamino, di(C₁-C₆)alkylamino, an alkyl group, a substituted alkyl group, —(C₃-C₈) cycloalkyl group, a substituted —(C₃-C₈) cycloalkyl group, —(C₃-C₈) cycloalkyl-(C₁-C₆)alkyl group, —O-alkyl group, a substituted —O-alkyl group, —O—(C₃-C₈) cycloalkyl group, a substituted —O—(C₃-C₈) cycloalkyl group, a phenyl group, a fused-phenyl group, a phenyl group substituted with one or more independently selected halogen, —OCH₃, nitro, or dimethyl amino groups, a substituted fused-phenyl group substituted with one or more independently selected halogen, —OCH₃, nitro, or dimethyl amino groups, a —(C₁-C₈) alkyl-phenyl group, —O—(C₀-C₈) alkyl-phenyl group, —(C₃-C₆) alkylene group, —O—(C₃-C₆) alkylene group, —(C₃-C₆) alkynyl group, —O—(C₃-C₆) alkynyl group, —CO(C₁-C₆) alkyl group, —NHCO(C₁-C₆) alkyl group, —NHSO₂(C₁-C₆) alkyl group, —SO₂(C₁-C₆) alkyl group, or —SO₂(C₀-C₆)alkyl-phenyl group;
and with the further proviso that in said compound of Formula (III), Z¹ is not a furyl group or a substituted furyl group when D² and Z² are independently selected from the group consisting of hydrogen and methyl;
and with the further proviso that in said compound comprising structure (III), when D² is —CH₃ and Z² is an optionally substituted phenyl, Z¹ is not selected from the group consisting of —R³³, OR³⁴, and —NR³⁵R³⁶,
wherein
R³³ is hydrogen, optionally substituted (C₃-C₈)cycloalkyl, optionally substituted phenyl, optionally substituted heteroaryl or phenyl-(C₁-C₄) alkyl which is optionally substituted on the phenyl ring, (C₁-C₆)alkyl, (C₂-C₆)alkenyl, (C₂-C₆)alkynyl, the three latter radicals independently being optionally substituted with one or more radicals selected from the group consisting of halogen, (C₁-C₄)alkoxy, (C₁-C₄)alkylthio and NR³⁷R³⁸;
R³⁴ is (C₁-C₆)alkyl, (C₂-C₆)alkenyl, (C₂-C₆)alkynyl, the three latter radicals independently optionally substituted with one or more radicals selected from the group consisting of halogen, (C₁-C₄)alkoxy, (C₁-C₄)alkylthio and NR³⁷R³⁸, or R³⁴ is (C₃-C₆)cycloalkyl optionally substituted with one or more groups selected from the group consisting of halogen, (C₁-C₄)alkyl and (C₁-C₄)alkoxy, and (C₃-C₆)-cycloalkyl-(C₁-C₃)alkyl;
R³⁵ and R³⁶ are hydrogen, (C₁-C₆)alkyl, (C₂-C₆)alkenyl, (C₂-C₆)alkynyl, the 3 latter groups independently optionally substituted by one or more halogen radicals;
R³⁷ is independently selected from the group consisting of hydrogen, (C₁-C₄)alkyl, ((C₁-C₄)alkyl)carbonyl, ((C₁-C₄)alkoxy)carbonyl and CHO;
R³⁸ is independently selected from the group consisting of H and (C₁-C₄)alkyl;
and with the further proviso that in said compound of Formula (III), Z¹ and Z² are not both selected from the group consisting of hydrogen and alkyl;
and with the further proviso that in said compound of Formula (III), Z² is not hydrogen when D¹ and D² are both hydrogen;
and with the further proviso that in said compound of Formula (III), Z¹ is not (C₁-C₆) alkyl, (C₁-C₆) alkoxy, pyridyl, or aryl when D¹ and D² are both hydrogen and when Z² is selected from the group consisting of hydrogen, a (C₁-C₆) alkyl group optionally substituted with 1 to 3 substituents independently selected from the group consisting of hydroxyl, (C₁-C₆) alkoxyl, carboxyl, (C₁-C₆) alkoxycarbonyl, lower alkylthio, fluorine, chlorine, bromine, iodine, amino, mono- or di-substituted amino, phthalimido and nitrogen-containing heterocyclic groups, a (C₃-C₈) cycloalkyl group, a (C₂-C₆) alkenyl group, an arylalkyl group optionally substituted with from 1 to 5 substituents independently selected from the group consisting of (C₁-C₆) alkyl, hydroxyl, (C₁-C₆) alkoxy, halogen, nitro, and amino groups, an aryl group optionally substituted with from 1 to 5 independently selected substituents selected from the group consisting of (C₁-C₆) alkyl, hydroxyl, (C₁-C₆) alkoxy, halogen, nitro, and amino groups, a substituted or unsubstituted heterocyclic group, a (C₁-C₆) alkanoyl group, a benzoyl or a naphthoyl group either of which is optionally substituted with from 1 to 5 independently selected substituents selected from the group consisting of (C₁-C₆) alkyl, hydroxyl, (C₁-C₆) alkoxy, halogen, nitro, and amino groups, a pyridylcarbonyl group, a (C₁-C₆) alkoxycarbonyl group or an amino group optionally substituted with (C₁-C₆) alkyl, arylalkyl having 7 to 15 carbon atoms, substituted or unsubstituted aryl, (C₁-C₆) alkanoyl, optionally substituted aroyl, and (C₁-C₆) alkylidene groups;
and with the further proviso that in said compound of Formula (III), Z¹ is not —O—(CH₂)ₜ—CH₃, wherein t is 0-4 and Z² is halophenyl;
and with the further proviso that in said compound of Formula (III), Z¹ is not a substituted pyridyl, pyrimidinyl, pyrazinyl, pyridazinyl group substituted with —N(R²¹)-(optionally substituted phenyl), wherein said R²¹ is hydrogen, aryl, formyl, (C₁-C₆)alkylcarbonyl, (C₁-C₆)alkyl, (C₁-C₆)alkyloxycarbonyl, (C₁-C₆)alkyl substituted with formyl, (C₁-C₆)alkylcarbonyl, (C₁-C₆)alkyloxycarbonyl, (C₁-C₆)alkylcarbonyloxy, or (C₁-C₆)alkyloxy(C₁-C₆)alkylcarbonyl optionally substituted with (C₁-C₆)alkyloxycarbonyl;
and with the further proviso that in said compound of Formula (III), Z¹ is not a 3-(4-trifluoromethyl-pyridinyl) group or a 3-(4-trifluoromethyl-pyridinyl N-oxide) group;
and with the further proviso that in said compound of Formula (III), Z¹ is not a substituted or unsubstituted group selected from the group consisting of

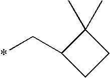

and a —(CH₂)ₜ-adamantanyl group, wherein t is 0-4;
and with the further proviso that in said compound of Formula (III), Z² is not a

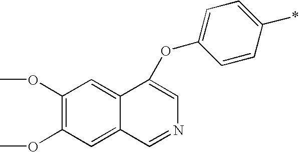

group, which is unsubstituted, or which is substituted with one or more substituents independently selected from the group consisting of halogens and alkyl groups;
and with the further proviso that in said compound of Formula (III), Z² is not a substituted or unsubstituted group selected from the group consisting of:

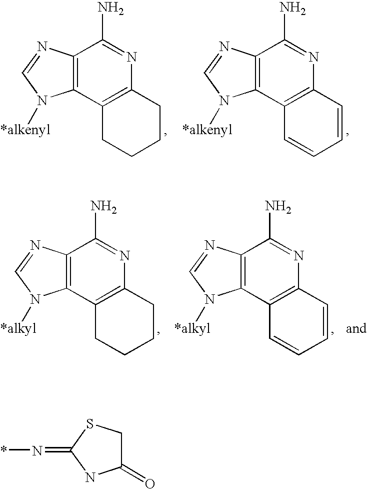

and with the further proviso that said compound of Formula (III), is not a compound set forth in Appendix A, Appendix B, or Appendix C.

In another embodiment, a compound of the present invention is a replicase complex defect inducer identified by the method of screening a test compound for replicase complex defect inducer activity comprising: providing a test compound; contacting the test compound with a cell expressing an isolated HCV replicase complex that comprises an NS3 protein with a mutation at or within about 15 angstroms of C16; and identifying the test compound as an inducer of an HCV replicase complex defect when the cell or HCV replicase complex is resistant to the test compound, wherein said compound is a compound of Formula (III)

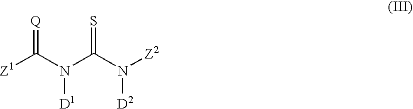

wherein
Q is oxygen or sulfur;
D¹ and D² are independently selected from the group consisting of hydrogen and methyl;
with the proviso that said compound of Formula (III) is not a compound of the formula (III-a):

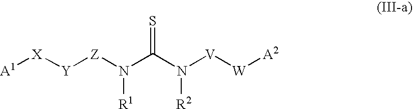

wherein

A₁ and A₂ are independently optionally substituted C₁-C₁₂ alkyl, optionally substituted mono- or di-(C₁-C₈ alkyl)amino, optionally substituted C₂-C₁₂ alkenyl, optionally substituted C₃-C₈ cycloalkyl, an optionally substituted partially unsaturated or aromatic carbocyclic group, or an optionally substituted saturated, partially unsaturated, or aromatic heterocyclic group, wherein at least one of A₁ and A₂ is an optionally substituted aromatic carbocyclic group or an optionally substituted aromatic heterocyclic group;

X and W are independently O, S, NR, or absent, wherein R is hydrogen, optionally substituted (C₁-C₆)alkyl, or optionally substituted aryl(C₀-C₄)alkyl;

V is (C₁-C₆)alkyl, (C₂-C₆)alkenyl, (C₃-C₇)cycloalkyl, or absent, wherein when V is absent, W is absent; and

Y is (C₁-C₆) alkyl, (C₁-C₆)alkyl substituted with (C₃-C₇)cycloalkyl, (C₂-C₆) alkenyl, (C₃-C₇)cycloalkyl, or absent; and

Z is carbonyl, thiocarbonyl, imino, or C₁-C₆ alkylimino; and

R₁ and R₂ are independently hydrogen or methyl;

and with the further proviso that said compound of Formula (III) is not a compound of the formula (III-b):

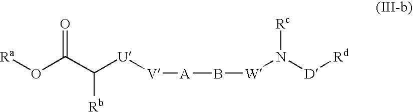

wherein
R^{a} is hydrogen, a substituted or unsubstituted alkyl or cycloalkyl group, a substituted or unsubstituted aryl group or a saturated or unsaturated, optionally substituted heterocyclic group;
R^{b} is hydrogen, a substituted or unsubstituted alkyl or cycloalkyl group, a substituted or unsubstituted aryl group, a saturated or unsaturated, optionally substituted heterocyclic group, an optionally substituted alkenyl group, an optionally substituted alkinyl group, —NR^{2′}SO₂R^{2″}, —NR^{2′}COOR^{2′}, —NR^{2′}COR^{2′}, —NR^{2′}CON(R^{2′})₂, or —N R^{2′}CSN(R^{2′})₂;
R^{2′} is hydrogen, a substituted or unsubstituted alkyl or cycloalkyl group, a substituted or unsubstituted aryl group or a saturated or unsaturated, optionally substituted heterocyclic group;
R^{2″} is a substituted or unsubstituted alkyl, alkenyl or cycloalkyl group, a substituted or unsubstituted aryl group, or a saturated or unsaturated, optionally substituted heterocyclic group;
U′ is a direct bond or a substituted or unsubstituted alkylene group;
V′ is a substituted or unsubstituted alkylene group, —NR^{2′}CO—, or —NR^{2′}SO₂—;
A and B are each independently an optionally substituted 1,3- or 1,4-bridging phenylene group or an optionally substituted 2,4- or 2,5-bridging thienylene group;
W′ is a direct bond or an optionally substituted alkylene group;
D′ is a direct bond or

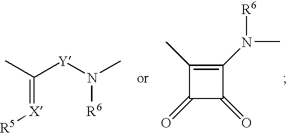

R^{c} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{d}, Y′, R⁵, or R⁶, if present, with formation of an optionally substituted heterocyclic ring system which includes the nitrogen atom to which R^{c} is bonded, and may be saturated or unsaturated and may contain further heteroatoms;
R^{d} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{c}, Y, R⁵ or R⁶, if present, with formation of an optionally substituted heterocyclic ring system which includes the nitrogen atom to which R^{d} is bonded and may be saturated or unsaturated and may contain further heteroatoms;

### X′ is CHNO₂, CHCN, O, N or S;

Y′ is a direct bond or an optionally substituted alkylene or alkine group;
R⁵ is absent, or is hydrogen, a substituted or unsubstituted alkyl or cycloalkyl group, —NO₂, —CN, —COR^{5′}, —COOR^{5′}, or is connected to one of R^{c}, Y′, R^{d}, or R⁶, if present, with formation of an optionally substituted carbocyclic or heterocyclic ring system which includes X′ and can be saturated or unsaturated and may optionally contain one or more additional heteroatoms;
R^{5′} is hydrogen, a substituted or unsubstituted alkyl or cycloalkyl group, a substituted or unsubstituted aryl group or a saturated or unsaturated, optionally substituted heterocyclic group which may optionally contain one or more additional heteroatoms;
R⁶ is a arylcarbonyl group, or a heteroarylcarbonyl group;
and wherein if A is a phenylene group and V′ is —NR^{2′}CO— or NR^{2′} SO₂—, D′ is not a direct bond and X′ is not N;
and with the further proviso that said compound of Formula (III) is not a compound of the formula (III-c)

X^{1″}—Y^{1″}-Z^{1″}-W^{1″} (III-c)

wherein
X^{1″} is optionally substituted aryl, optionally substituted (C₁-C₈)alkyl-aryl, optionally substituted heteroaryl, or optionally substituted heteroarylalkyl;
Y^{1″} is —NR^{A}CSNR^{A}CO—, wherein R^{A} is independently hydrogen or lower alkyl;
Z^{1″} is optionally substituted phenylene, optionally substituted monocyclic heteroarylene, optionally substituted monocyclic non-aromatic heterocycle-diyl, or optionally substituted monocyclic cycloalkane-diyl;
W^{1″} is a group represented by the formula:

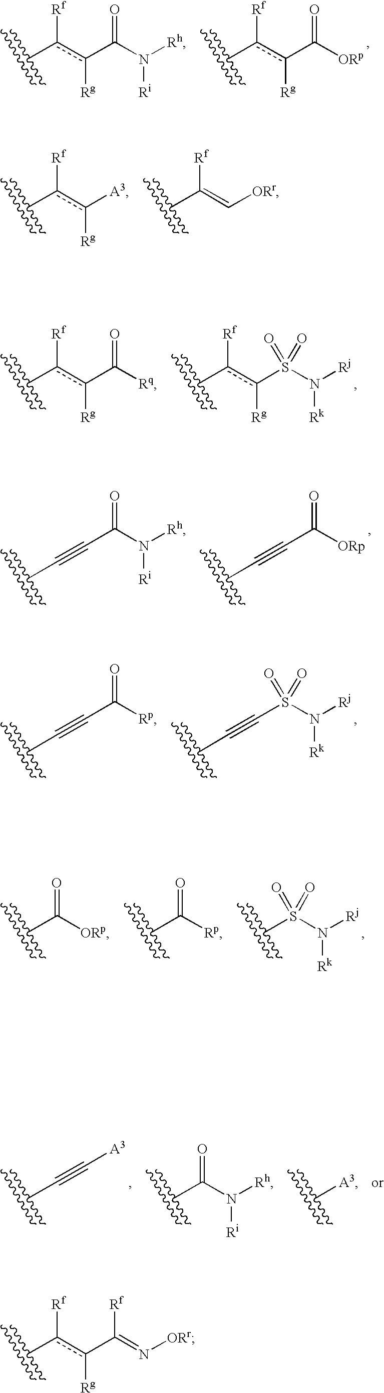

wherein
R^{f}, R^{g}, R^{h}, Rⁱ, R^{j}, and R^{k} are each independently hydrogen, halogen, optionally substituted lower alkyl, optionally substituted lower alkyloxy, optionally substituted lower alkylthio, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, optionally substituted non-aromatic heterocyclic group, or optionally substituted amino;
R^{p}, R^{q}, and R^{r} are each independently hydrogen, optionally substituted lower alkyl, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, or optionally substituted non-aromatic heterocyclic group;
A³ is an optionally substituted aryl or an optionally substituted heteroaryl group;
and with the further proviso that in said compound of Formula (III), Z¹ is not a 3-pyridyl group or a substituted 3-pyridyl group when Z² is —OH, —NH₂, —(C₁-C₆)alkylamino, di(C₁-C₆)alkylamino, an alkyl group, a substituted alkyl group, —(C₃-C₈) cycloalkyl group, a substituted —(C₃-C₈) cycloalkyl group, —(C₃-C₈) cycloalkyl-(C₁-C₆)alkyl group, —O-alkyl group, a substituted —O-alkyl group, —O—(C₃-C₈) cycloalkyl group, a substituted —O—(C₃-C₈) cycloalkyl group, a phenyl group, a fused-phenyl group, a phenyl group substituted with one or more independently selected halogen, —OCH₃, nitro, or dimethyl amino groups, a substituted fused-phenyl group substituted with one or more independently selected halogen, —OCH₃, nitro, or dimethyl amino groups, a —(C₁-C₈) alkyl-phenyl group, —O—(C₀-C₈) alkyl-phenyl group, —(C₃-C₆) alkylene group, —O—(C₃-C₆) alkylene group, —(C₃-C₆) alkynyl group, —O—(C₃-C₆) alkynyl group, —CO(C₁-C₆) alkyl group, —NHCO(C₁-C₆) alkyl group, —NHSO₂(C₁-C₆) alkyl group, —SO₂(C₁-C₆) alkyl group, or —SO₂(C₀-C₆)alkyl-phenyl group;
and with the further proviso that in said compound of Formula (III), Z¹ is not a furyl group or a substituted furyl group when D² and Z² are independently selected from the group consisting of hydrogen and methyl;
and with the further proviso that in said compound comprising structure (III), when D² is —CH₃ and Z² is an optionally substituted phenyl, Z¹ is not selected from the group consisting of —R³³, OR³⁴, and —NR³⁵R³⁶,
wherein
R³³ is hydrogen, optionally substituted (C₃-C₈)cycloalkyl, optionally substituted phenyl, optionally substituted heteroaryl or phenyl-(C₁-C₄) alkyl which is optionally substituted on the phenyl ring, (C₁-C₆)alkyl, (C₂-C₆)alkenyl, (C₂-C₆)alkynyl, the three latter radicals independently being optionally substituted with one or more radicals selected from the group consisting of halogen, (C₁-C₄)alkoxy, (C₁-C₄)alkylthio and NR³⁷R³⁸;
R³⁴ is (C₁-C₆)alkyl, (C₂-C₆)alkenyl, (C₂-C₆)alkynyl, the three latter radicals independently optionally substituted with one or more radicals selected from the group consisting of halogen, (C₁-C₄)alkoxy, (C₁-C₄)alkylthio and NR³⁷R³⁸, or R³⁴ is (C₃-C₆)cycloalkyl optionally substituted with one or more groups selected from the group consisting of halogen, (C₁-C₄)alkyl and (C₁-C₄)alkoxy, and (C₃-C₆)-cycloalkyl-(C₁-C₃)alkyl;
R³⁵ and R³⁶ are hydrogen, (C₁-C₆)alkyl, (C₂-C₆)alkenyl, (C₂-C₆)alkynyl, the 3 latter groups independently optionally substituted by one or more halogen radicals;
R³⁷ is independently selected from the group consisting of hydrogen, (C₁-C₄)alkyl, ((C₁-C₄)alkyl)carbonyl, ((C₁-C₄)alkoxy)carbonyl and CHO;
R³⁸ is independently selected from the group consisting of H and (C₁-C₄)alkyl;
and with the further proviso that in said compound of Formula (III), Z² is not hydrogen when D¹ and D² are both hydrogen;
and with the further proviso that in said compound of Formula (III), Z¹ is not (C₁-C₆) alkyl, (C₁-C₆) alkoxy, pyridyl, or aryl when D¹ and D² are both hydrogen and when Z² is selected from the group consisting of hydrogen, a (C₁-C₆) alkyl group optionally substituted with 1 to 3 substituents independently selected from the group consisting of hydroxyl, (C₁-C₆) alkoxyl, carboxyl, (C₁-C₆) alkoxycarbonyl, lower alkylthio, fluorine, chlorine, bromine, iodine, amino, mono- or di-substituted amino, phthalimido and nitrogen-containing heterocyclic groups, a (C₃-C₈) cycloalkyl group, a (C₂-C₆) alkenyl group, an arylalkyl group optionally substituted with from 1 to 5 substituents independently selected from the group consisting of (C₁-C₆) alkyl, hydroxyl, (C₁-C₆) alkoxy, halogen, nitro, and amino groups, an aryl group optionally substituted with from 1 to 5 independently selected substituents selected from the group consisting of (C₁-C₆) alkyl, hydroxyl, (C₁-C₆) alkoxy, halogen, nitro, and amino groups, a substituted or unsubstituted heterocyclic group, a (C₁-C₆) alkanoyl group, a benzoyl or a naphthoyl group either of which is optionally substituted with from 1 to 5 independently selected substituents selected from the group consisting of (C₁-C₆) alkyl, hydroxyl, (C₁-C₆) alkoxy, halogen, nitro, and amino groups, a pyridylcarbonyl group, a (C₁-C₆) alkoxycarbonyl group or an amino group optionally substituted with (C₁-C₆) alkyl, arylalkyl having 7 to 15 carbon atoms, substituted or unsubstituted aryl, (C₁-C₆) alkanoyl, optionally substituted aroyl, and (C₁-C₆) alkylidene groups;
and with the further proviso that in said compound of Formula (III), Z¹ is not —O—(CH₂)ₜ—CH₃, wherein t is 0-4 and Z² is halophenyl;
and with the further proviso that in said compound of Formula (III), Z¹ is not a substituted pyridyl, pyrimidinyl, pyrazinyl, pyridazinyl group substituted with —N(R²¹)-(optionally substituted phenyl), wherein said R²¹ is hydrogen, aryl, formyl, (C₁-C₆)alkylcarbonyl, (C₁-C₆)alkyl, (C₁-C₆)alkyloxycarbonyl, (C₁-C₆)alkyl substituted with formyl, (C₁-C₆)alkylcarbonyl, (C₁-C₆)alkyloxycarbonyl, (C₁-C₆)alkylcarbonyloxy, or (C₁-C₆)alkyloxy(C₁-C₆)alkylcarbonyl optionally substituted with (C₁-C₆)alkyloxycarbonyl;
and with the further proviso that in said compound of Formula (III), Z¹ is not a 3-(4-trifluoromethyl-pyridinyl) group or a 3-(4-trifluoromethyl-pyridinyl N-oxide) group;
and with the further proviso that in said compound of Formula (III), Z¹ is not a substituted or unsubstituted group selected from the group consisting of

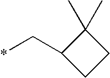

and a —(CH₂)ₜ-adamantanyl group, wherein t is 0-4;
and with the further proviso that in said compound of Formula (II), Z² is not a

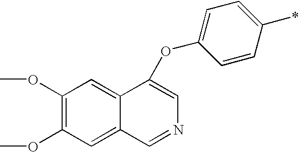

group, which is unsubstituted, or which is substituted with one or more substituents independently selected from the group consisting of halogens and alkyl groups;
and with the further proviso that in said compound of Formula (III), Z² is not a substituted or unsubstituted group selected from the group consisting of:

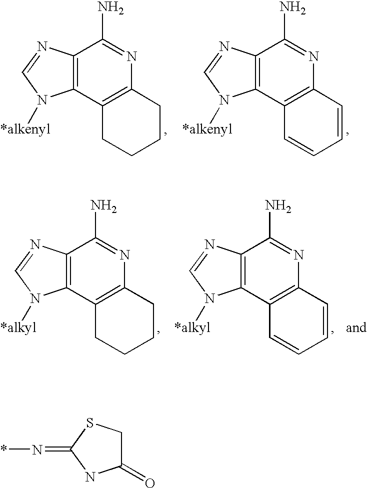

and with the further proviso that said compound of Formula (III), is not a compound set forth in Appendix A, Appendix B, or Appendix C.

In another embodiment, a compound of the present invention is a replicase complex defect inducer identified by the method of screening a test compound for replicase complex defect inducer activity comprising: providing a test compound; contacting the test compound with a cell expressing an isolated HCV polyprotein that comprises an NS3 protein with a mutation that is at or within about 15 angstroms of C16; and identifying the test compound as an inducer of an HCV replicase complex defect when the cell or HCV polyprotein is resistant to the test compound, wherein said compound is a compound of Formula (III)

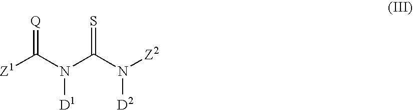

wherein
Q is oxygen or sulfur;
D¹ and D² are independently selected from the group consisting of hydrogen and methyl;
with the proviso that said compound of Formula (III) is not a compound of the formula (III-a):

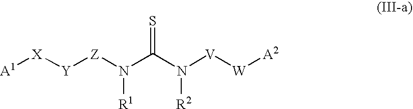

wherein

A₁ and A₂ are independently optionally substituted C₁-C₁₂ alkyl, optionally substituted mono- or di-(C₁-C₈ alkyl)amino, optionally substituted C₂-C₁₂ alkenyl, optionally substituted C₃-C₈ cycloalkyl, an optionally substituted partially unsaturated or aromatic carbocyclic group, or an optionally substituted saturated, partially unsaturated, or aromatic heterocyclic group, wherein at least one of A₁ and A₂ is an optionally substituted aromatic carbocyclic group or an optionally substituted aromatic heterocyclic group;

X and W are independently O, S, NR, or absent, wherein R is hydrogen, optionally substituted (C₁-C₆)alkyl, or optionally substituted aryl(C₀-C₄)alkyl;

V is (C₁-C₆)alkyl, (C₂-C₆)alkenyl, (C₃-C₇)cycloalkyl, or absent, wherein when V is absent, W is absent; and

Y is (C₁-C₆) alkyl, (C₁-C₆)alkyl substituted with (C₃-C₇)cycloalkyl, (C₂-C₆) alkenyl, (C₃-C₇)cycloalkyl, or absent; and

Z is carbonyl, thiocarbonyl, imino, or C₁-C₆ alkylimino; and

R₁ and R₂ are independently hydrogen or methyl;

and with the further proviso that said compound of Formula (III) is not a compound of the formula (III-b):

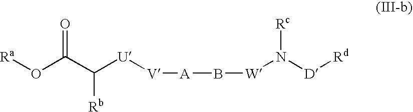

wherein
R^{a} is hydrogen, a substituted or unsubstituted alkyl or cycloalkyl group, a substituted or unsubstituted aryl group or a saturated or unsaturated, optionally substituted heterocyclic group;
R^{b} is hydrogen, a substituted or unsubstituted alkyl or cycloalkyl group, a substituted or unsubstituted aryl group, a saturated or unsaturated, optionally substituted heterocyclic group, an optionally substituted alkenyl group, an optionally substituted alkinyl group, —NR^{2′}SO₂R^{2″}, —NR^{2′}COOR^{2′}, —NR^{2′}COR^{2′}, —NR^{2′}CON(R^{2′})₂, or —N R^{2′}CSN(R^{2′})₂;
R^{2′} is hydrogen, a substituted or unsubstituted alkyl or cycloalkyl group, a substituted or unsubstituted aryl group or a saturated or unsaturated, optionally substituted heterocyclic group;
R^{2″} is a substituted or unsubstituted alkyl, alkenyl or cycloalkyl group, a substituted or unsubstituted aryl group, or a saturated or unsaturated, optionally substituted heterocyclic group;
U′ is a direct bond or a substituted or unsubstituted alkylene group;
V′ is a substituted or unsubstituted alkylene group, —NR^{2′}CO—, or —NR^{2′}SO₂—;
A and B are each independently an optionally substituted 1,3- or 1,4-bridging phenylene group or an optionally substituted 2,4- or 2,5-bridging thienylene group;
W′ is a direct bond or an optionally substituted alkylene group;
D′ is a direct bond or

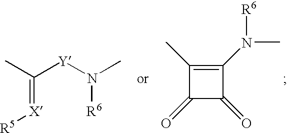

R^{c} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{d}, Y′, R⁵, or R⁶, if present, with formation of an optionally substituted heterocyclic ring system which includes the nitrogen atom to which R^{c} is bonded, and may be saturated or unsaturated and may contain further heteroatoms;
R^{d} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{c}, Y, R⁵ or R⁶, if present, with formation of an optionally substituted heterocyclic ring system which includes the nitrogen atom to which R^{d} is bonded and may be saturated or unsaturated and may contain further heteroatoms;

### X′ is CHNO₂, CHCN, O, N or S;

Y′ is a direct bond or an optionally substituted alkylene or alkine group;
R⁵ is absent, or is hydrogen, a substituted or unsubstituted alkyl or cycloalkyl group, —NO₂, —CN, —COR^{5′}, —COOR^{5′}, or is connected to one of R^{c}, Y′, R^{d}, or R⁶, if present, with formation of an optionally substituted carbocyclic or heterocyclic ring system which includes X′ and can be saturated or unsaturated and may optionally contain one or more additional heteroatoms;
R^{5′} is hydrogen, a substituted or unsubstituted alkyl or cycloalkyl group, a substituted or unsubstituted aryl group or a saturated or unsaturated, optionally substituted heterocyclic group which may optionally contain one or more additional heteroatoms;
R⁶ is a arylcarbonyl group, or a heteroarylcarbonyl group;
and wherein if A is a phenylene group and V′ is —NR^{2′}CO— or —NR^{2′} SO₂—, D′ is not a direct bond and X′ is not N;
and with the further proviso that said compound of Formula (III) is not a compound of the formula (III-c)

X^{1″}—Y^{1″}-Z^{1″}-W^{1″} (III-c)

wherein
X^{1″} is optionally substituted aryl, optionally substituted (C₁-C₈)alkyl-aryl, optionally substituted heteroaryl, or optionally substituted heteroarylalkyl;
Y^{1″} is —NR^{A}CSNR^{A}CO—, wherein R^{A} is independently hydrogen or lower alkyl;
Z^{1″} is optionally substituted phenylene, optionally substituted monocyclic heteroarylene, optionally substituted monocyclic non-aromatic heterocycle-diyl, or optionally substituted monocyclic cycloalkane-diyl;
W^{1″} is a group represented by the formula:

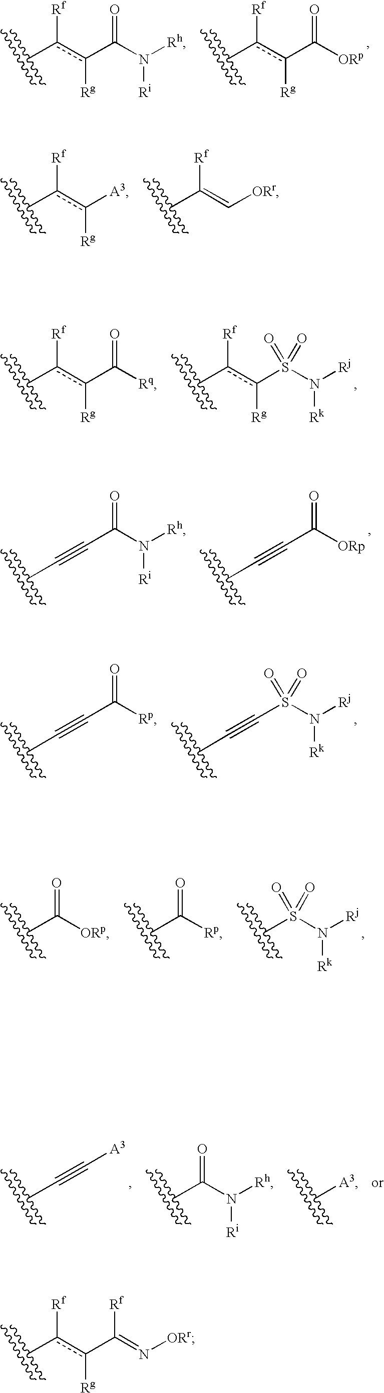

wherein
R^{f}, R^{g}, R^{h}, Rⁱ, R^{j}, and R^{k} are each independently hydrogen, halogen, optionally substituted lower alkyl, optionally substituted lower alkyloxy, optionally substituted lower alkylthio, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, optionally substituted non-aromatic heterocyclic group, or optionally substituted amino;
R^{p}, R^{q}, and R^{r} are each independently hydrogen, optionally substituted lower alkyl, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, or optionally substituted non-aromatic heterocyclic group;
A³ is an optionally substituted aryl or an optionally substituted heteroaryl group;
and with the further proviso that in said compound of Formula (III), Z¹ is not a 3-pyridyl group or a substituted 3-pyridyl group when Z² is —OH, —NH₂, —(C₁-C₆)alkylamino, di(C₁-C₆)alkylamino, an alkyl group, a substituted alkyl group, —(C₃-C₈) cycloalkyl group, a substituted —(C₃-C₈) cycloalkyl group, —(C₃-C₈) cycloalkyl-(C₁-C₆)alkyl group, —O-alkyl group, a substituted —O-alkyl group, —O—(C₃-C₈) cycloalkyl group, a substituted —O—(C₃-C₈) cycloalkyl group, a phenyl group, a fused-phenyl group, a phenyl group substituted with one or more independently selected halogen, —OCH₃, nitro, or dimethyl amino groups, a substituted fused-phenyl group substituted with one or more independently selected halogen, —OCH₃, nitro, or dimethyl amino groups, a —(C₁-C₈) alkyl-phenyl group, —O—(C₀-C₈) alkyl-phenyl group, —(C₃-C₆) alkylene group, —O—(C₃-C₆) alkylene group, —(C₃-C₆) alkynyl group, —O—(C₃-C₆) alkynyl group, —CO(C₁-C₆) alkyl group, —NHCO(C₁-C₆) alkyl group, —NHSO₂(C₁-C₆) alkyl group, —SO₂(C₁-C₆) alkyl group, or —SO₂(C₀-C₆)alkyl-phenyl group;
and with the further proviso that in said compound of Formula (III), Z¹ is not a furyl group or a substituted furyl group when D² and Z² are independently selected from the group consisting of hydrogen and methyl;
and with the further proviso that in said compound comprising structure (III), when D² is —CH₃ and Z² is an optionally substituted phenyl, Z¹ is not selected from the group consisting of —R³³, OR³⁴, and —NR³⁵R³⁶,
wherein
R³³ is hydrogen, optionally substituted (C₃-C₈)cycloalkyl, optionally substituted phenyl, optionally substituted heteroaryl or phenyl-(C₁-C₄) alkyl which is optionally substituted on the phenyl ring, (C₁-C₆)alkyl, (C₂-C₆)alkenyl, (C₂-C₆)alkynyl, the three latter radicals independently being optionally substituted with one or more radicals selected from the group consisting of halogen, (C₁-C₄)alkoxy, (C₁-C₄)alkylthio and NR³⁷R³⁸;
R³⁴ is (C₁-C₆)alkyl, (C₂-C₆)alkenyl, (C₂-C₆)alkynyl, the three latter radicals independently optionally substituted with one or more radicals selected from the group consisting of halogen, (C₁-C₄)alkoxy, (C₁-C₄)alkylthio and NR³⁷R³⁸, or R³⁴ is (C₃-C₆)cycloalkyl optionally substituted with one or more groups selected from the group consisting of halogen, (C₁-C₄)alkyl and (C₁-C₄)alkoxy, and (C₃-C₆)-cycloalkyl-(C₁-C₃)alkyl;
R³⁵ and R³⁶ are hydrogen, (C₁-C₆)alkyl, (C₂-C₆)alkenyl, (C₂-C₆)alkynyl, the 3 latter groups independently optionally substituted by one or more halogen radicals;
R³⁷ is independently selected from the group consisting of hydrogen, (C₁-C₄)alkyl, ((C₁-C₄)alkyl)carbonyl, ((C₁-C₄)alkoxy)carbonyl and CHO;
R³⁸ is independently selected from the group consisting of H and (C₁-C₄)alkyl;
and with the further proviso that in said compound of Formula (III), Z¹ and Z² are not both selected from the group consisting of hydrogen and alkyl;
and with the further proviso that in said compound of Formula (III), Z² is not hydrogen when D¹ and D² are both hydrogen;
and with the further proviso that in said compound of Formula (III), Z¹ is not (C₁-C₆) alkyl, (C₁-C₆) alkoxy, pyridyl, or aryl when D¹ and D² are both hydrogen and when Z² is selected from the group consisting of hydrogen, a (C₁-C₆) alkyl group optionally substituted with 1 to 3 substituents independently selected from the group consisting of hydroxyl, (C₁-C₆) alkoxyl, carboxyl, (C₁-C₆) alkoxycarbonyl, lower alkylthio, fluorine, chlorine, bromine, iodine, amino, mono- or di-substituted amino, phthalimido and nitrogen-containing heterocyclic groups, a (C₃-C₈) cycloalkyl group, a (C₂-C₆) alkenyl group, an arylalkyl group optionally substituted with from 1 to 5 substituents independently selected from the group consisting of (C₁-C₆) alkyl, hydroxyl, (C₁-C₆) alkoxy, halogen, nitro, and amino groups, an aryl group optionally substituted with from 1 to 5 independently selected substituents selected from the group consisting of (C₁-C₆) alkyl, hydroxyl, (C₁-C₆) alkoxy, halogen, nitro, and amino groups, a substituted or unsubstituted heterocyclic group, a (C₁-C₆) alkanoyl group, a benzoyl or a naphthoyl group either of which is optionally substituted with from 1 to 5 independently selected substituents selected from the group consisting of (C₁-C₆) alkyl, hydroxyl, (C₁-C₆) alkoxy, halogen, nitro, and amino groups, a pyridylcarbonyl group, a (C₁-C₆) alkoxycarbonyl group or an amino group optionally substituted with (C₁-C₆) alkyl, arylalkyl having 7 to 15 carbon atoms, substituted or unsubstituted aryl, (C₁-C₆) alkanoyl, optionally substituted aroyl, and (C₁-C₆) alkylidene groups;
and with the further proviso that in said compound of Formula (III), Z¹ is not —O—(CH₂)ₜ—CH₃, wherein t is 0-4 and Z² is halophenyl;
and with the further proviso that in said compound of Formula (III), Z¹ is not a substituted pyridyl, pyrimidinyl, pyrazinyl, pyridazinyl group substituted with —N(R²¹)-(optionally substituted phenyl), wherein said R²¹ is hydrogen, aryl, formyl, (C₁-C₆)alkylcarbonyl, (C₁-C₆)alkyl, (C₁-C₆)alkyloxycarbonyl, (C₁-C₆)alkyl substituted with formyl, (C₁-C₆)alkylcarbonyl, (C₁-C₆)alkyloxycarbonyl, (C₁-C₆)alkylcarbonyloxy, or (C₁-C₆)alkyloxy(C₁-C₆)alkylcarbonyl optionally substituted with (C₁-C₆)alkyloxycarbonyl;
and with the further proviso that in said compound of Formula (III), Z¹ is not a 3-(4-trifluoromethyl-pyridinyl) group or a 3-(4-trifluoromethyl-pyridinyl N-oxide) group;
and with the further proviso that in said compound of Formula (III), Z¹ is not a substituted or unsubstituted group selected from the group consisting of

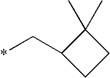

and a —(CH₂)ₜ-adamantanyl group, wherein t is 0-4;
and with the further proviso that in said compound of Formula (III), Z² is not a

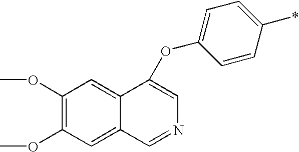

group, which is unsubstituted, or which is substituted with one or more substituents independently selected from the group consisting of halogens and alkyl groups;
and with the further proviso that in said compound of Formula (III), Z² is not a substituted or unsubstituted group selected from the group consisting of:

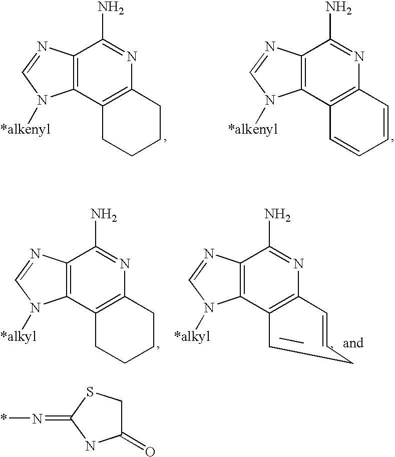

and with the further proviso that said compound of Formula (III), is not a compound set forth in Appendix A, Appendix B, or Appendix C.

In another embodiment, a compound of the present invention is a replicase complex defect inducer identified by the method of screening a test compound for replicase complex defect inducer activity comprising: providing a test compound; contacting the test compound with a cell that expresses an HCV virion; and identifying the test compound as an inducer of an HCV replicase complex defect,

wherein the compound is a compound of Formula (III)

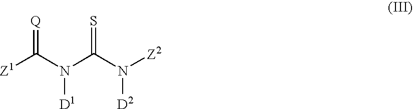

wherein
Q is oxygen or sulfur;
D¹ and D² are independently selected from the group consisting of hydrogen and methyl;
with the proviso that said compound of Formula (III) is not a compound of the formula (III-a):

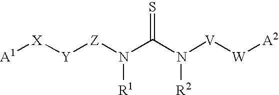

wherein

A₁ and A₂ are independently optionally substituted C₁-C₁₂ alkyl, optionally substituted mono- or di-(C₁-C₈ alkyl)amino, optionally substituted C₂-C₁₂ alkenyl, optionally substituted C₃-C₈ cycloalkyl, an optionally substituted partially unsaturated or aromatic carbocyclic group, or an optionally substituted saturated, partially unsaturated, or aromatic heterocyclic group, wherein at least one of A₁ and A₂ is an optionally substituted aromatic carbocyclic group or an optionally substituted aromatic heterocyclic group;

X and W are independently O, S, NR, or absent, wherein R is hydrogen, optionally substituted (C₁-C₆)alkyl, or optionally substituted aryl(C₀-C₄)alkyl;

V is (C₁-C₆)alkyl, (C₂-C₆)alkenyl, (C₃-C₇)cycloalkyl, or absent, wherein when V is absent, W is absent; and

Y is (C₁-C₆) alkyl, (C₁-C₆)alkyl substituted with (C₃-C₇)cycloalkyl, (C₂-C₆) alkenyl, (C₃-C₇)cycloalkyl, or absent; and

Z is carbonyl, thiocarbonyl, imino, or C₁-C₆ alkylimino; and

R₁ and R₂ are independently hydrogen or methyl;

and with the further proviso that said compound of Formula (III) is not a compound of Formula (III-b):

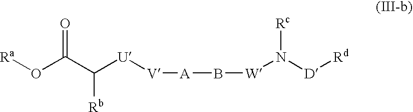

wherein
R^{a} is hydrogen, a substituted or unsubstituted alkyl or cycloalkyl group, a substituted or unsubstituted aryl group or a saturated or unsaturated, optionally substituted heterocyclic group;
R^{b} is hydrogen, a substituted or unsubstituted alkyl or cycloalkyl group, a substituted or unsubstituted aryl group, a saturated or unsaturated, optionally substituted heterocyclic group, an optionally substituted alkenyl group, an optionally substituted alkinyl group, —NR^{2′}SO₂R^{2′}, —NR^{2′}COOR^{2′}, —NR^{2′}COR^{2′}, —NR^{2′}CON(R^{2′})₂, or —N R^{2′}CSN(R^{2′})₂;
R² is hydrogen, a substituted or unsubstituted alkyl or cycloalkyl group, a substituted or unsubstituted aryl group or a saturated or unsaturated, optionally substituted heterocyclic group;
R^{2″} is a substituted or unsubstituted alkyl, alkenyl or cycloalkyl group, a substituted or unsubstituted aryl group, or a saturated or unsaturated, optionally substituted heterocyclic group;
U′ is a direct bond or a substituted or unsubstituted alkylene group;
V′ is a substituted or unsubstituted alkylene group, —NR^{2′}CO—, or —NR^{2′}SO₂—;
A and B are each independently an optionally substituted 1,3- or 1,4-bridging phenylene group or an optionally substituted 2,4- or 2,5-bridging thienylene group;
W′ is a direct bond or an optionally substituted alkylene group;
D′ is a direct bond or

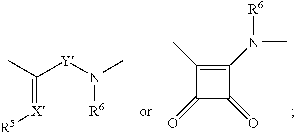

R^{c} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{d}, Y′, R⁵, or R⁶, if present, with formation of an optionally substituted heterocyclic ring system which includes the nitrogen atom to which R^{c} is bonded, and may be saturated or unsaturated and may contain further heteroatoms;
R^{d} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{c}, Y, R⁵ or R⁶, if present, with formation of an optionally substituted heterocyclic ring system which includes the nitrogen atom to which R^{d} is bonded and may be saturated or unsaturated and may contain further heteroatoms;

### X′ is CHNO₂, CHCN, O, N or S;

Y′ is a direct bond or an optionally substituted alkylene or alkine group;
R⁵ is absent, or is hydrogen, a substituted or unsubstituted alkyl or cycloalkyl group, —NO₂, —CN, —COR^{5′}, —COOR^{5′}, or is connected to one of R^{c}, Y′, R^{d}, or R⁶, if present, with formation of an optionally substituted carbocyclic or heterocyclic ring system which includes X′ and can be saturated or unsaturated and may optionally contain one or more additional heteroatoms;
R^{5′} is hydrogen, a substituted or unsubstituted alkyl or cycloalkyl group, a substituted or unsubstituted aryl group or a saturated or unsaturated, optionally substituted heterocyclic group which may optionally contain one or more additional heteroatoms;
R⁶ is a arylcarbonyl group, or a heteroarylcarbonyl group;
and wherein if A is a phenylene group and V′ is —NR^{2′}CO— or —NR^{2′} SO₂—, D′ is not a direct bond and X′ is not N;
and with the further proviso that said compound of Formula (III) is not a compound of Formula (III-c)

X^{1″}—Y^{1″}-Z^{1″}-W^{1″} (III-c)

wherein
X^{1″} is optionally substituted aryl, optionally substituted (C₁-C₈)alkyl-aryl, optionally substituted heteroaryl, or optionally substituted heteroarylalkyl;
Y^{1″} is —NR^{A}CSNR^{A}CO—, wherein R^{A} is independently hydrogen or lower alkyl;
Z^{1″} is optionally substituted phenylene, optionally substituted monocyclic heteroarylene, optionally substituted monocyclic non-aromatic heterocycle-diyl, or optionally substituted monocyclic cycloalkane-diyl;
W^{1″} is a group represented by the formula:

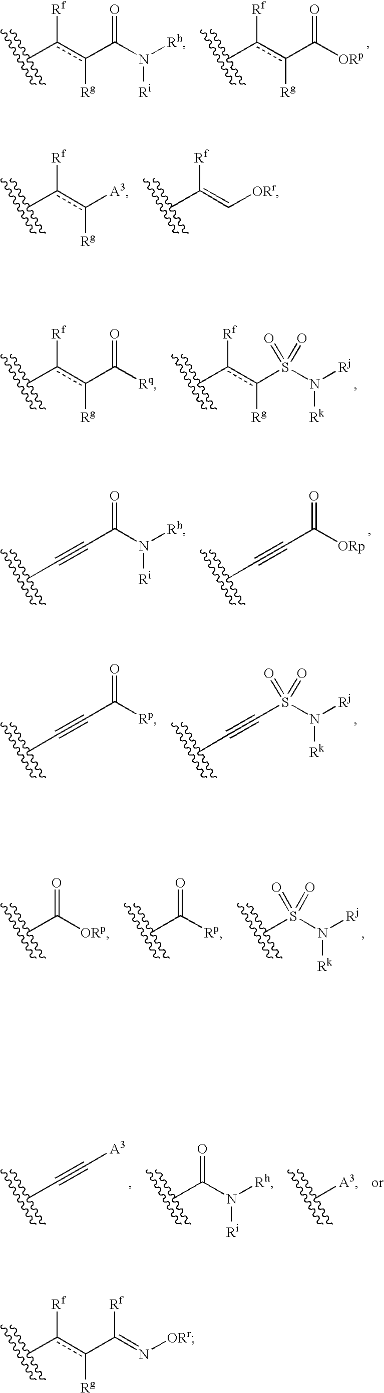

wherein
R^{f}, R^{g}, R^{h}, Rⁱ, R^{j}, and R^{k} are each independently hydrogen, halogen, optionally substituted lower alkyl, optionally substituted lower alkyloxy, optionally substituted lower alkylthio, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, optionally substituted non-aromatic heterocyclic group, or optionally substituted amino;
R^{p}, R^{q}, and R^{r} are each independently hydrogen, optionally substituted lower alkyl, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, or optionally substituted non-aromatic heterocyclic group;
A³ is an optionally substituted aryl or an optionally substituted heteroaryl group;
and with the further proviso that in said compound of Formula (III), Z¹ is not a 3-pyridyl group or a substituted 3-pyridyl group when Z² is —OH, —NH₂, —(C₁-C₆)alkylamino, di(C₁-C₆)alkylamino, an alkyl group, a substituted alkyl group, —(C₃-C₈) cycloalkyl group, a substituted —(C₃-C₈) cycloalkyl group, —(C₃-C₈) cycloalkyl-(C₁-C₆)alkyl group, —O-alkyl group, a substituted —O-alkyl group, —O—(C₃-C₈) cycloalkyl group, a substituted —O—(C₃-C₈) cycloalkyl group, a phenyl group, a fused-phenyl group, a phenyl group substituted with one or more independently selected halogen, —OCH₃, nitro, or dimethyl amino groups, a substituted fused-phenyl group substituted with one or more independently selected halogen, —OCH₃, nitro, or dimethyl amino groups, a —(C₁-C₈) alkyl-phenyl group, —O—(C₀-C₈) alkyl-phenyl group, —(C₃-C₆) alkylene group, —O—(C₃-C₆) alkylene group, —(C₃-C₆) alkynyl group, —O—(C₃-C₆) alkynyl group, —CO(C₁-C₆) alkyl group, —NHCO(C₁-C₆) alkyl group, —NHSO₂(C₁-C₆) alkyl group, —SO₂(C₁-C₆) alkyl group, or —SO₂(C₀-C₆)alkyl-phenyl group;
and with the further proviso that in said compound of Formula (III), Z¹ is not a furyl group or a substituted furyl group when D² and Z² are independently selected from the group consisting of hydrogen and methyl;
and with the further proviso that in said compound comprising structure (III), when D² is —CH₃ and Z² is an optionally substituted phenyl, Z¹ is not selected from the group consisting of —R³³, OR³⁴, and —NR³⁵R³⁶,
wherein
R³³ is hydrogen, optionally substituted (C₃-C₈)cycloalkyl, optionally substituted phenyl, optionally substituted heteroaryl or phenyl-(C₁-C₄) alkyl which is optionally substituted on the phenyl ring, (C₁-C₆)alkyl, (C₂-C₆)alkenyl, (C₂-C₆)alkynyl, the three latter radicals independently being optionally substituted with one or more radicals selected from the group consisting of halogen, (C₁-C₄)alkoxy, (C₁-C₄)alkylthio and NR³⁷R³⁸;
R³⁴ is (C₁-C₆)alkyl, (C₂-C₆)alkenyl, (C₂-C₆)alkynyl, the three latter radicals independently optionally substituted with one or more radicals selected from the group consisting of halogen, (C₁-C₄)alkoxy, (C₁-C₄)alkylthio and NR³⁷R³⁸, or R³⁴ is (C₃-C₆)cycloalkyl optionally substituted with one or more groups selected from the group consisting of halogen, (C₁-C₄)alkyl and (C₁-C₄)alkoxy, and (C₃-C₆)-cycloalkyl-(C₁-C₃)alkyl;
R³⁵ and R³⁶ are hydrogen, (C₁-C₆)alkyl, (C₂-C₆)alkenyl, (C₂-C₆)alkynyl, the 3 latter groups independently optionally substituted by one or more halogen radicals;
R³⁷ is independently selected from the group consisting of hydrogen, (C₁-C₄)alkyl, ((C₁-C₄)alkyl)carbonyl, ((C₁-C₄)alkoxy)carbonyl and CHO;
R³⁸ is independently selected from the group consisting of H and (C₁-C₄)alkyl;
and with the further proviso that in said compound of Formula (III), Z¹ and Z² are not both selected from the group consisting of hydrogen and alkyl;
and with the further proviso that in said compound of Formula (III), Z² is not hydrogen when D¹ and D² are both hydrogen;
and with the further proviso that in said compound of Formula (III), Z¹ is not (C₁-C₆) alkyl, (C₁-C₆) alkoxy, pyridyl, or aryl when D¹ and D² are both hydrogen and when Z² is selected from the group consisting of hydrogen, a (C₁-C₆) alkyl group optionally substituted with 1 to 3 substituents independently selected from the group consisting of hydroxyl, (C₁-C₆) alkoxyl, carboxyl, (C₁-C₆) alkoxycarbonyl, lower alkylthio, fluorine, chlorine, bromine, iodine, amino, mono- or di-substituted amino, phthalimido and nitrogen-containing heterocyclic groups, a (C₃-C₈) cycloalkyl group, a (C₂-C₆) alkenyl group, an arylalkyl group optionally substituted with from 1 to 5 substituents independently selected from the group consisting of (C₁-C₆) alkyl, hydroxyl, (C₁-C₆) alkoxy, halogen, nitro, and amino groups, an aryl group optionally substituted with from 1 to 5 independently selected substituents selected from the group consisting of (C₁-C₆) alkyl, hydroxyl, (C₁-C₆) alkoxy, halogen, nitro, and amino groups, a substituted or unsubstituted heterocyclic group, a (C₁-C₆) alkanoyl group, a benzoyl or a naphthoyl group either of which is optionally substituted with from 1 to 5 independently selected substituents selected from the group consisting of (C₁-C₆) alkyl, hydroxyl, (C₁-C₆) alkoxy, halogen, nitro, and amino groups, a pyridylcarbonyl group, a (C₁-C₆) alkoxycarbonyl group or an amino group optionally substituted with (C₁-C₆) alkyl, arylalkyl having 7 to 15 carbon atoms, substituted or unsubstituted aryl, (C₁-C₆) alkanoyl, optionally substituted aroyl, and (C₁-C₆) alkylidene groups;
and with the further proviso that in said compound of Formula (III), Z¹ is not —O—(CH₂)ₜ—CH₃, wherein t is 0-4 and Z² is halophenyl;
and with the further proviso that in said compound of Formula (III), Z¹ is not a substituted pyridyl, pyrimidinyl, pyrazinyl, pyridazinyl group substituted with —N(R²¹)-(optionally substituted phenyl), wherein said R²¹ is hydrogen, aryl, formyl, (C₁-C₆)alkylcarbonyl, (C₁-C₆)alkyl, (C₁-C₆)alkyloxycarbonyl, (C₁-C₆)alkyl substituted with formyl, (C₁-C₆)alkylcarbonyl, (C₁-C₆)alkyloxycarbonyl, (C₁-C₆)alkylcarbonyloxy, or (C₁-C₆)alkyloxy(C₁-C₆)alkylcarbonyl optionally substituted with (C₁-C₆)alkyloxycarbonyl;
and with the further proviso that in said compound of Formula (III), Z¹ is not a 3-(4-trifluoromethyl-pyridinyl) group or a 3-(4-trifluoromethyl-pyridinyl N-oxide) group;
and with the further proviso that in said compound of Formula (III), Z¹ is not a substituted or unsubstituted group selected from the group consisting of

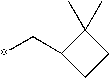

and a —(CH₂)ₜ-adamantanyl group, wherein t is 0-4;
and with the further proviso that in said compound of Formula (III), Z² is not a

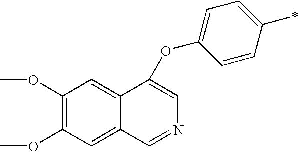

group, which is unsubstituted, or which is substituted with one or more substituents independently selected from the group consisting of halogens and alkyl groups;
and with the further proviso that in said compound of Formula (III), Z² is not a substituted or unsubstituted group selected from the group consisting of:

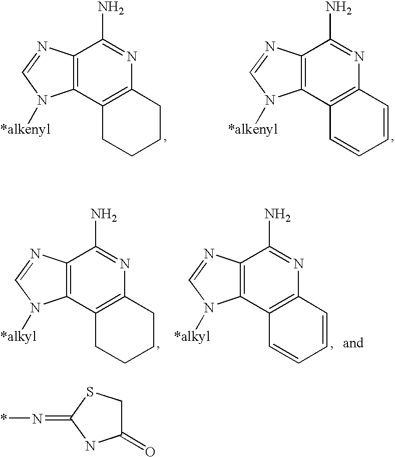

and with the further proviso that said compound of Formula (III), is not a compound set forth in Appendix A, Appendix B, or Appendix C.

In another embodiment, a compound of the present invention is a replicase complex defect inducer identified by the method of screening a test compound for replicase complex defect inducer activity comprising: providing a test compound; contacting the test compound with a cell that expresses an HCV replicon; and identifying the test compound as an inducer of an HCV replicase complex defect,

wherein the compound is a compound of Formula (III)

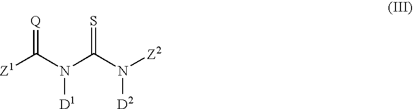

wherein
Q is oxygen or sulfur;
D¹ and D² are independently selected from the group consisting of hydrogen and methyl;
with the proviso that said compound of Formula (III) is not a compound of the formula (III-a):

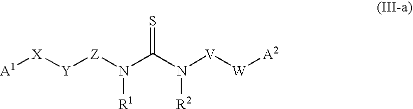

wherein

A₁ and A₂ are independently optionally substituted C₁-C₁₂ alkyl, optionally substituted mono- or di-(C₁-C₈ alkyl)amino, optionally substituted C₂-C₁₂ alkenyl, optionally substituted C₃-C₈ cycloalkyl, an optionally substituted partially unsaturated or aromatic carbocyclic group, or an optionally substituted saturated, partially unsaturated, or aromatic heterocyclic group, wherein at least one of A₁ and A₂ is an optionally substituted aromatic carbocyclic group or an optionally substituted aromatic heterocyclic group;

X and W are independently O, S, NR, or absent, wherein R is hydrogen, optionally substituted (C₁-C₆)alkyl, or optionally substituted aryl(C₀-C₄)alkyl;

V is (C₁-C₆)alkyl, (C₂-C₆)alkenyl, (C₃-C₇)cycloalkyl, or absent, wherein when V is absent, W is absent; and

Y is (C₁-C₆) alkyl, (C₁-C₆)alkyl substituted with (C₃-C₇)cycloalkyl, (C₂-C₆) alkenyl, (C₃-C₇)cycloalkyl, or absent; and

Z is carbonyl, thiocarbonyl, imino, or C₁-C₆ alkylimino; and

R₁ and R₂ are independently hydrogen or methyl;

and with the further proviso that said compound of Formula (III) is not a compound of Formula (III-b):

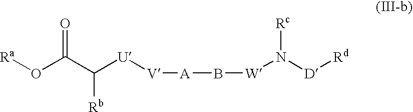

wherein
R^{a} is hydrogen, a substituted or unsubstituted alkyl or cycloalkyl group, a substituted or unsubstituted aryl group or a saturated or unsaturated, optionally substituted heterocyclic group;
R^{b} is hydrogen, a substituted or unsubstituted alkyl or cycloalkyl group, a substituted or unsubstituted aryl group, a saturated or unsaturated, optionally substituted heterocyclic group, an optionally substituted alkenyl group, an optionally substituted alkinyl group, —NR^{2′}SO₂R^{2″}, —NR^{2′}COOR^{2′}, —NR^{2′}COR^{2′}, —NR^{2′}CON(R^{2′})₂, or —N R^{2′}CSN(R^{2′})₂;
R^{2′} is hydrogen, a substituted or unsubstituted alkyl or cycloalkyl group, a substituted or unsubstituted aryl group or a saturated or unsaturated, optionally substituted heterocyclic group;
R^{2″} is a substituted or unsubstituted alkyl, alkenyl or cycloalkyl group, a substituted or unsubstituted aryl group, or a saturated or unsaturated, optionally substituted heterocyclic group;
U′ is a direct bond or a substituted or unsubstituted alkylene group;
V′ is a substituted or unsubstituted alkylene group, —NR^{2′}CO—, or —NR^{2′}SO₂—;
A and B are each independently an optionally substituted 1,3- or 1,4-bridging phenylene group or an optionally substituted 2,4- or 2,5-bridging thienylene group;
W′ is a direct bond or an optionally substituted alkylene group;
D′ is a direct bond or

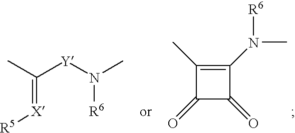

R^{c} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{d}, Y′, R⁵, or R⁶, if present, with formation of an optionally substituted heterocyclic ring system which includes the nitrogen atom to which R^{c} is bonded, and may be saturated or unsaturated and may contain further heteroatoms;
R^{d} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{c}, Y, R⁵ or R⁶, if present, with formation of an optionally substituted heterocyclic ring system which includes the nitrogen atom to which R^{d} is bonded and may be saturated or unsaturated and may contain further heteroatoms;

### X′ is CHNO₂, CHCN, O, N or S;

Y′ is a direct bond or an optionally substituted alkylene or alkine group;
R⁵ is absent, or is hydrogen, a substituted or unsubstituted alkyl or cycloalkyl group, —NO₂, —CN, —COR^{5′}, —COOR^{5′}, or is connected to one of R^{c}, Y′, R^{d}, or R⁶, if present, with formation of an optionally substituted carbocyclic or heterocyclic ring system which includes X′ and can be saturated or unsaturated and may optionally contain one or more additional heteroatoms;
R^{5′} is hydrogen, a substituted or unsubstituted alkyl or cycloalkyl group, a substituted or unsubstituted aryl group or a saturated or unsaturated, optionally substituted heterocyclic group which may optionally contain one or more additional heteroatoms;
R⁶ is a arylcarbonyl group, or a heteroarylcarbonyl group;
and wherein if A is a phenylene group and V′ is —NR^{2′}CO— or —NR^{2′}SO₂—, D′ is not a direct bond and X′ is not N;
and with the further proviso that said compound of Formula (III) is not a compound of Formula (III-c)

X^{1″}—Y^{1″}-Z^{1″}-W^{1″} (III-c)

wherein
X^{1″} is optionally substituted aryl, optionally substituted (C₁-C₈)alkyl-aryl, optionally substituted heteroaryl, or optionally substituted heteroarylalkyl;
Y^{1″} is —NR^{A}CSNR^{A}CO—, wherein R^{A} is independently hydrogen or lower alkyl;
Z^{1″} is optionally substituted phenylene, optionally substituted monocyclic heteroarylene, optionally substituted monocyclic non-aromatic heterocycle-diyl, or optionally substituted monocyclic cycloalkane-diyl;
W^{1″} is a group represented by the formula:

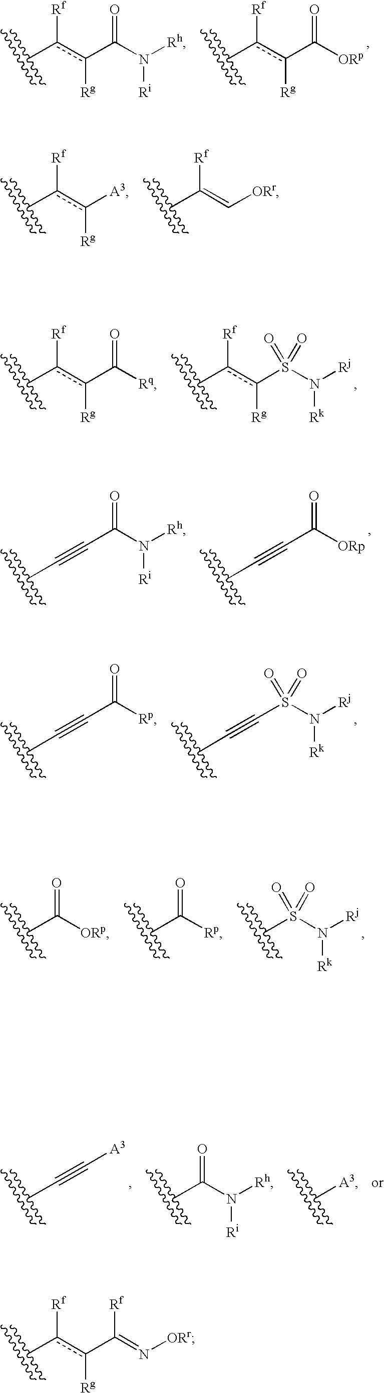

wherein
R^{f}, R^{g}, R^{h}, Rⁱ, R^{j}, and R^{k} are each independently hydrogen, halogen, optionally substituted lower alkyl, optionally substituted lower alkyloxy, optionally substituted lower alkylthio, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, optionally substituted non-aromatic heterocyclic group, or optionally substituted amino;
R^{p}, R^{q}, and R^{r} are each independently hydrogen, optionally substituted lower alkyl, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, or optionally substituted non-aromatic heterocyclic group;
A³ is an optionally substituted aryl or an optionally substituted heteroaryl group;
and with the further proviso that in said compound of Formula (III), Z¹ is not a 3-pyridyl group or a substituted 3-pyridyl group when Z² is —OH, —NH₂, —(C₁-C₆)alkylamino, di(C₁-C₆)alkylamino, an alkyl group, a substituted alkyl group, —(C₃-C₈) cycloalkyl group, a substituted —(C₃-C₈) cycloalkyl group, —(C₃-C₈) cycloalkyl-(C₁-C₆)alkyl group, —O-alkyl group, a substituted —O-alkyl group, —O—(C₃-C₈) cycloalkyl group, a substituted —O—(C₃-C₈) cycloalkyl group, a phenyl group, a fused-phenyl group, a phenyl group substituted with one or more independently selected halogen, —OCH₃, nitro, or dimethyl amino groups, a substituted fused-phenyl group substituted with one or more independently selected halogen, —OCH₃, nitro, or dimethyl amino groups, a —(C₁-C₈) alkyl-phenyl group, —O—(C₀-C₈) alkyl-phenyl group, —(C₃-C₆) alkylene group, —O—(C₃-C₆) alkylene group, —C₃-C₆) alkynyl group, —O—(C₃-C₆) alkynyl group, —CO(C₁-C₆) alkyl group, —NHCO(C₁-C₆) alkyl group, —NHSO₂(C₁-C₆) alkyl group, —SO₂(C₁-C₆) alkyl group, or —SO₂(C₀-C₆)alkyl-phenyl group;
and with the further proviso that in said compound of Formula (III), Z¹ is not a furyl group or a substituted furyl group when D² and Z² are independently selected from the group consisting of hydrogen and methyl;
and with the further proviso that in said compound comprising structure (III), when D² is —CH₃ and Z² is an optionally substituted phenyl, Z¹ is not selected from the group consisting of —R³³, OR³⁴, and —NR³⁵R³⁶,
wherein
R³³ is hydrogen, optionally substituted (C₃-C₈)cycloalkyl, optionally substituted phenyl, optionally substituted heteroaryl or phenyl-(C₁-C₄) alkyl which is optionally substituted on the phenyl ring, (C₁-C₆)alkyl, (C₂-C₆)alkenyl, (C₂-C₆)alkynyl, the three latter radicals independently being optionally substituted with one or more radicals selected from the group consisting of halogen, (C₁-C₄)alkoxy, (C₁-C₄)alkylthio and
R³⁴ is (C₁-C₆)alkyl, (C₂-C₆)alkenyl, (C₂-C₆)alkynyl, the three latter radicals independently optionally substituted with one or more radicals selected from the group consisting of halogen, (C₁-C₄)alkoxy, (C₁-C₄)alkylthio and NR³⁷R³⁸, or R³⁴ is (C₃-C₆)cycloalkyl optionally substituted with one or more groups selected from the group consisting of halogen, (C₁-C₄)alkyl and (C₁-C₄)alkoxy, and (C₃-C₆)-cycloalkyl-(C₁-C₃)alkyl;
R³⁵ and R³⁶ are hydrogen, (C₁-C₆)alkyl, (C₂-C₆)alkenyl, (C₂-C₆)alkynyl, the 3 latter groups independently optionally substituted by one or more halogen radicals;
R³⁷ is independently selected from the group consisting of hydrogen, (C₁-C₄)alkyl, ((C₁-C₄)alkyl)carbonyl, ((C₁-C₄)alkoxy)carbonyl and CHO;
R³⁸ is independently selected from the group consisting of H and (C₁-C₄)alkyl;
and with the further proviso that in said compound of Formula (III), Z¹ and Z² are not both selected from the group consisting of hydrogen and alkyl;
and with the further proviso that in said compound of Formula (III), Z² is not hydrogen when D¹ and D² are both hydrogen;
and with the further proviso that in said compound of Formula (III), Z¹ is not (C₁-C₆) alkyl, (C₁-C₆) alkoxy, pyridyl, or aryl when D¹ and D² are both hydrogen and when Z² is selected from the group consisting of hydrogen, a (C₁-C₆) alkyl group optionally substituted with 1 to 3 substituents independently selected from the group consisting of hydroxyl, (C₁-C₆) alkoxyl, carboxyl, (C₁-C₆) alkoxycarbonyl, lower alkylthio, fluorine, chlorine, bromine, iodine, amino, mono- or di-substituted amino, phthalimido and nitrogen-containing heterocyclic groups, a (C₃-C₈) cycloalkyl group, a (C₂-C₆) alkenyl group, an arylalkyl group optionally substituted with from 1 to 5 substituents independently selected from the group consisting of (C₁-C₆) alkyl, hydroxyl, (C₁-C₆) alkoxy, halogen, nitro, and amino groups, an aryl group optionally substituted with from 1 to 5 independently selected substituents selected from the group consisting of (C₁-C₆) alkyl, hydroxyl, (C₁-C₆) alkoxy, halogen, nitro, and amino groups, a substituted or unsubstituted heterocyclic group, a (C₁-C₆) alkanoyl group, a benzoyl or a naphthoyl group either of which is optionally substituted with from 1 to 5 independently selected substituents selected from the group consisting of (C₁-C₆) alkyl, hydroxyl, (C₁-C₆) alkoxy, halogen, nitro, and amino groups, a pyridylcarbonyl group, a (C₁-C₆) alkoxycarbonyl group or an amino group optionally substituted with (C₁-C₆) alkyl, arylalkyl having 7 to 15 carbon atoms, substituted or unsubstituted aryl, (C₁-C₆) alkanoyl, optionally substituted aroyl, and (C₁-C₆) alkylidene groups;
and with the further proviso that in said compound of Formula (III), Z¹ is not —O—(CH₂)ₜ—CH₃, wherein t is 0-4 and Z² is halophenyl;
and with the further proviso that in said compound of Formula (III), Z¹ is not a substituted pyridyl, pyrimidinyl, pyrazinyl, pyridazinyl group substituted with —N(R²¹)-(optionally substituted phenyl), wherein said R²¹ is hydrogen, aryl, formyl, (C₁-C₆)alkylcarbonyl, (C₁-C₆)alkyl, (C₁-C₆)alkyloxycarbonyl, (C₁-C₆)alkyl substituted with formyl, (C₁-C₆)alkylcarbonyl, (C₁-C₆)alkyloxycarbonyl, (C₁-C₆)alkylcarbonyloxy, or (C₁-C₆)alkyloxy(C₁-C₆)alkylcarbonyl optionally substituted with (C₁-C₆)alkyloxycarbonyl;
and with the further proviso that in said compound of Formula (III), Z¹ is not a 3-(4-trifluoromethyl-pyridinyl) group or a 3-(4-trifluoromethyl-pyridinyl N-oxide) group;
and with the further proviso that in said compound of Formula (III), Z¹ is not a substituted or unsubstituted group selected from the group consisting of

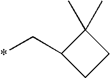

and a —(CH₂)ₜ-adamantanyl group, wherein t is 0-4;
and with the further proviso that in said compound of Formula (III), Z² is not a

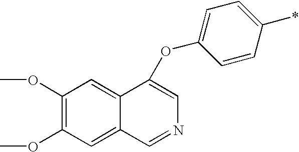

group, which is unsubstituted, or which is substituted with one or more substituents independently selected from the group consisting of halogens and alkyl groups;
and with the further proviso that in said compound of Formula (III), Z² is not a substituted or unsubstituted group selected from the group consisting of:

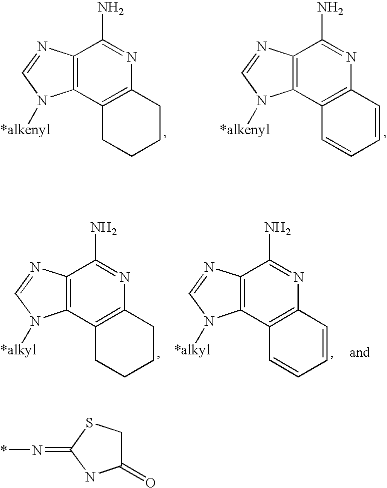

and with the further proviso that said compound of Formula (III), is not a compound set forth in Appendix A, Appendix B, or Appendix C.

In another embodiment, a compound of the present invention is a replicase complex defect inducer identified by the method of screening a test compound for replicase complex defect inducer activity comprising: providing a test compound; contacting the test compound with a cell that expresses an isolated HCV replicase complex, and identifying the test compound as an inducer of an HCV replicase complex defect, wherein the compound is a compound of Formula (III)

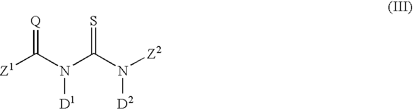

wherein
Q is oxygen or sulfur;
D¹ and D² are independently selected from the group consisting of hydrogen and methyl;
with the proviso that said compound of Formula (III) is not a compound of the formula (III-a):

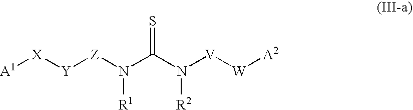

wherein

A₁ and A₂ are independently optionally substituted C₁-C₁₂ alkyl, optionally substituted mono- or di-(C₁-C₈ alkyl)amino, optionally substituted C₂-C₁₂ alkenyl, optionally substituted C₃-C₈ cycloalkyl, an optionally substituted partially unsaturated or aromatic carbocyclic group, or an optionally substituted saturated, partially unsaturated, or aromatic heterocyclic group, wherein at least one of A₁ and A₂ is an optionally substituted aromatic carbocyclic group or an optionally substituted aromatic heterocyclic group;

X and W are independently O, S, NR, or absent, wherein R is hydrogen, optionally substituted (C₁-C₆)alkyl, or optionally substituted aryl(C₀-C₄)alkyl;

V is (C₁-C₆)alkyl, (C₂-C₆)alkenyl, (C₃-C₇)cycloalkyl, or absent, wherein when V is absent, W is absent; and

Y is (C₁-C₆) alkyl, (C₁-C₆)alkyl substituted with (C₃-C₇)cycloalkyl, (C₂-C₆) alkenyl, (C₃-C₇)cycloalkyl, or absent; and

Z is carbonyl, thiocarbonyl, imino, or C₁-C₆ alkylimino; and

R₁ and R₂ are independently hydrogen or methyl;

and with the further proviso that said compound of Formula (III) is not a compound of the formula (III-b):

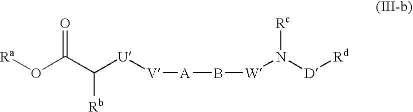

wherein
R^{a} is hydrogen, a substituted or unsubstituted alkyl or cycloalkyl group, a substituted or unsubstituted aryl group or a saturated or unsaturated, optionally substituted heterocyclic group;
R^{b} is hydrogen, a substituted or unsubstituted alkyl or cycloalkyl group, a substituted or unsubstituted aryl group, a saturated or unsaturated, optionally substituted heterocyclic group, an optionally substituted alkenyl group, an optionally substituted alkinyl group, —NR^{2′}SO₂R^{2″}, —NR^{2′}COOR^{2′}, —NR^{2′}COR^{2′}, —NR^{2′}CON(R^{2′})₂, or —N R^{2′}CSN(R^{2′})₂;
R^{2′} is hydrogen, a substituted or unsubstituted alkyl or cycloalkyl group, a substituted or unsubstituted aryl group or a saturated or unsaturated, optionally substituted heterocyclic group;
R^{2″} is a substituted or unsubstituted alkyl, alkenyl or cycloalkyl group, a substituted or unsubstituted aryl group, or a saturated or unsaturated, optionally substituted heterocyclic group;
U′ is a direct bond or a substituted or unsubstituted alkylene group;
V′ is a substituted or unsubstituted alkylene group, —NR^{2′}CO—, or —NR^{2′}SO₂—;
A and B are each independently an optionally substituted 1,3- or 1,4-bridging phenylene group or an optionally substituted 2,4- or 2,5-bridging thienylene group;
W′ is a direct bond or an optionally substituted alkylene group;
D′ is a direct bond or

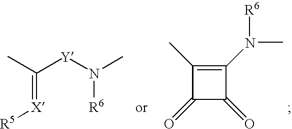

R^{c} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{d}, Y′, R⁵, or R⁶, if present, with formation of an optionally substituted heterocyclic ring system which includes the nitrogen atom to which R^{c} is bonded, and may be saturated or unsaturated and may contain further heteroatoms;
R^{d} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{c}, Y, R⁵ or R⁶, if present, with formation of an optionally substituted heterocyclic ring system which includes the nitrogen atom to which R^{d} is bonded and may be saturated or unsaturated and may contain further heteroatoms;

### X′ is CHNO₂, CHCN, O, N or S;

Y′ is a direct bond or an optionally substituted alkylene or alkine group;
R⁵ is absent, or is hydrogen, a substituted or unsubstituted alkyl or cycloalkyl group, —NO₂, —CN, —COR^{5′}, —COOR^{5′}, or is connected to one of R^{c}, Y′, R^{d}, or R⁶, if present, with formation of an optionally substituted carbocyclic or heterocyclic ring system which includes X′ and can be saturated or unsaturated and may optionally contain one or more additional heteroatoms;
R^{5′} is hydrogen, a substituted or unsubstituted alkyl or cycloalkyl group, a substituted or unsubstituted aryl group or a saturated or unsaturated, optionally substituted heterocyclic group which may optionally contain one or more additional heteroatoms;
R⁶ is a arylcarbonyl group, or a heteroarylcarbonyl group;
and wherein if A is a phenylene group and V′ is —NR^{2′}CO— or —NR^{2′} SO₂—, D′ is not a direct bond and X′ is not N;
and with the further proviso that said compound of Formula (III) is not a compound of the formula (III-c)

X^{1″}—Y^{1″}-Z^{1″}-W^{1″} (III-c)

wherein
X^{1″} is optionally substituted aryl, optionally substituted (C₁-C₈)alkyl-aryl, optionally substituted heteroaryl, or optionally substituted heteroarylalkyl;
Y^{1″} is —NR^{A}CSNR^{A}CO—, wherein R^{A} is independently hydrogen or lower alkyl;
Z^{1″} is optionally substituted phenylene, optionally substituted monocyclic heteroarylene, optionally substituted monocyclic non-aromatic heterocycle-diyl, or optionally substituted monocyclic cycloalkane-diyl;
W^{1″} is a group represented by the formula:

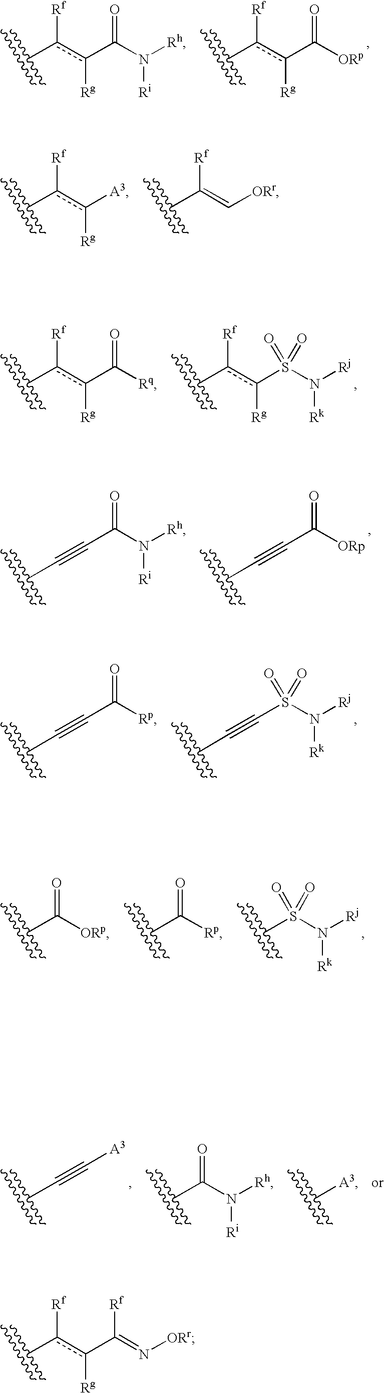

wherein
R^{f}, R^{g}, R^{h}, Rⁱ, R^{j}, and R^{k} are each independently hydrogen, halogen, optionally substituted lower alkyl, optionally substituted lower alkyloxy, optionally substituted lower alkylthio, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, optionally substituted non-aromatic heterocyclic group, or optionally substituted amino;
R^{p}, R^{q}, and R^{r} are each independently hydrogen, optionally substituted lower alkyl, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, or optionally substituted non-aromatic heterocyclic group;
A³ is an optionally substituted aryl or an optionally substituted heteroaryl group;
and with the further proviso that in said compound of Formula (III), Z¹ is not a 3-pyridyl group or a substituted 3-pyridyl group when Z² is —OH, —NH₂, —(C₁-C₆)alkylamino, di(C₁-C₆)alkylamino, an alkyl group, a substituted alkyl group, —(C₃-C₈) cycloalkyl group, a substituted —(C₃-C₈) cycloalkyl group, —(C₃-C₈) cycloalkyl-(C₁-C₆)alkyl group, —O-alkyl group, a substituted —O-alkyl group, —O—(C₃-C₈) cycloalkyl group, a substituted —O—(C₃-C₈) cycloalkyl group, a phenyl group, a fused-phenyl group, a phenyl group substituted with one or more independently selected halogen, —OCH₃, nitro, or dimethyl amino groups, a substituted fused-phenyl group substituted with one or more independently selected halogen, —OCH₃, nitro, or dimethyl amino groups, a —(C₀-C₈) alkyl-phenyl group, —O—(C₀-C₈) alkyl-phenyl group, —(C₃-C₆) alkylene group, —O—(C₃-C₆) alkylene group, —(C₃-C₆) alkynyl group, —O—(C₃-C₆) alkynyl group, —CO(C₁-C₆) alkyl group, —NHCO(C₁-C₆) alkyl group, —NHSO₂(C₁-C₆) alkyl group, —SO₂(C₁-C₆) alkyl group, or —SO₂(C₀-C₆)alkyl-phenyl group;
and with the further proviso that in said compound of Formula (III), Z¹ is not a furyl group or a substituted furyl group when D² and Z² are independently selected from the group consisting of hydrogen and methyl;
and with the further proviso that in said compound comprising structure (III), when D² is —CH₃ and Z² is an optionally substituted phenyl, Z¹ is not selected from the group consisting of —R³³, OR³⁴, and —NR³⁵R³⁶,
wherein
R³³ is hydrogen, optionally substituted (C₃-C₈)cycloalkyl, optionally substituted phenyl, optionally substituted heteroaryl or phenyl-(C₀-C₄) alkyl which is optionally substituted on the phenyl ring, (C₁-C₆)alkyl, (C₂-C₆)alkenyl, (C₂-C₆)alkynyl, the three latter radicals independently being optionally substituted with one or more radicals selected from the group consisting of halogen, (C₁-C₄)alkoxy, (C₁-C₄)alkylthio and NR³⁷R³⁸;
R³⁴ is (C₁-C₆)alkyl, (C₂-C₆)alkenyl, (C₂-C₆)alkynyl, the three latter radicals independently optionally substituted with one or more radicals selected from the group consisting of halogen, (C₁-C₄)alkoxy, (C₁-C₄)alkylthio and NR³⁷R³⁸, or R³⁴ is (C₃-C₆)cycloalkyl optionally substituted with one or more groups selected from the group consisting of halogen, (C₁-C₄)alkyl and (C₁-C₄)alkoxy, and (C₃-C₆)-cycloalkyl-(C₁-C₃)alkyl;
R³⁵ and R³⁶ are hydrogen, (C₁-C₆)alkyl, (C₂-C₆)alkenyl, (C₂-C₆)alkynyl, the 3 latter groups independently optionally substituted by one or more halogen radicals;
R³⁷ is independently selected from the group consisting of hydrogen, (C₁-C₄)alkyl, ((C₁-C₄)alkyl)carbonyl, ((C₁-C₄)alkoxy)carbonyl and CHO;
R³⁸ is independently selected from the group consisting of H and (C₁-C₄)alkyl;
and with the further proviso that in said compound of Formula (III), Z¹ and Z² are not both selected from the group consisting of hydrogen and alkyl;
and with the further proviso that in said compound of Formula (III), Z² is not hydrogen when D¹ and D² are both hydrogen;
and with the further proviso that in said compound of Formula (III), Z¹ is not (C₁-C₆) alkyl, (C₁-C₆) alkoxy, pyridyl, or aryl when D¹ and D² are both hydrogen and when Z² is selected from the group consisting of hydrogen, a (C₁-C₆) alkyl group optionally substituted with 1 to 3 substituents independently selected from the group consisting of hydroxyl, (C₁-C₆) alkoxyl, carboxyl, (C₁-C₆) alkoxycarbonyl, lower alkylthio, fluorine, chlorine, bromine, iodine, amino, mono- or di-substituted amino, phthalimido and nitrogen-containing heterocyclic groups, a (C₃-C₈) cycloalkyl group, a (C₂-C₆) alkenyl group, an arylalkyl group optionally substituted with from 1 to 5 substituents independently selected from the group consisting of (C₁-C₆) alkyl, hydroxyl, (C₁-C₆) alkoxy, halogen, nitro, and amino groups, an aryl group optionally substituted with from 1 to 5 independently selected substituents selected from the group consisting of (C₁-C₆) alkyl, hydroxyl, (C₁-C₆) alkoxy, halogen, nitro, and amino groups, a substituted or unsubstituted heterocyclic group, a (C₁-C₆) alkanoyl group, a benzoyl or a naphthoyl group either of which is optionally substituted with from 1 to 5 independently selected substituents selected from the group consisting of (C₁-C₆) alkyl, hydroxyl, (C₁-C₆) alkoxy, halogen, nitro, and amino groups, a pyridylcarbonyl group, a (C₁-C₆) alkoxycarbonyl group or an amino group optionally substituted with (C₁-C₆) alkyl, arylalkyl having 7 to 15 carbon atoms, substituted or unsubstituted aryl, (C₁-C₆) alkanoyl, optionally substituted aroyl, and (C₁-C₆) alkylidene groups;
and with the further proviso that in said compound of Formula (III), Z¹ is not —O—(CH₂)ₜ—CH₃, wherein t is 0-4 and Z² is halophenyl;
and with the further proviso that in said compound of Formula (III), Z¹ is not a substituted pyridyl, pyrimidinyl, pyrazinyl, pyridazinyl group substituted with —N(R²¹)-(optionally substituted phenyl), wherein said R²¹ is hydrogen, aryl, formyl, (C₁-C₆)alkylcarbonyl, (C₁-C₆)alkyl, (C₁-C₆)alkyloxycarbonyl, (C₁-C₆)alkyl substituted with formyl, (C₁-C₆)alkylcarbonyl, (C₁-C₆)alkyloxycarbonyl, (C₁-C₆)alkylcarbonyloxy, or (C₁-C₆)alkyloxy(C₁-C₆)alkylcarbonyl optionally substituted with (C₁-C₆)alkyloxycarbonyl;
and with the further proviso that in said compound of Formula (III), Z¹ is not a 3-(4-trifluoromethyl-pyridinyl) group or a 3-(4-trifluoromethyl-pyridinyl N-oxide) group;
and with the further proviso that in said compound of Formula (III), Z¹ is not a substituted or unsubstituted group selected from the group consisting of

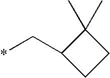

and a —(CH₂)ₜ-adamantanyl group, wherein t is 0-4;
and with the further proviso that in said compound of Formula (III), Z² is not a

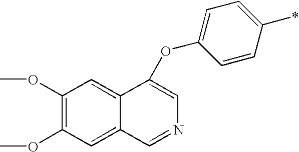

group, which is unsubstituted, or which is substituted with one or more substituents independently selected from the group consisting of halogens and alkyl groups;
and with the further proviso that in said compound of Formula (III), Z² is not a substituted or unsubstituted group selected from the group consisting of:

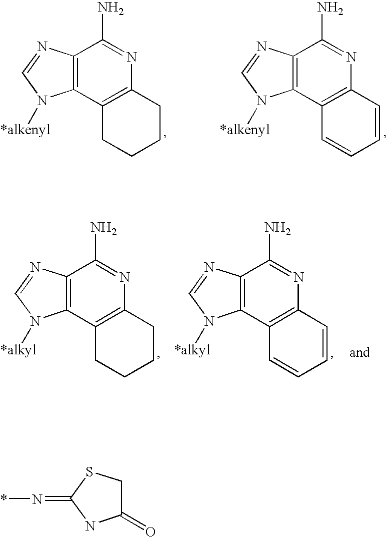

and with the further proviso that said compound of Formula (III), is not a compound set forth in Appendix A, Appendix B, or Appendix C.

In another embodiment, a compound of the present invention is a replicase complex defect inducer identified by the method of screening a test compound for replicase complex defect inducer activity comprising: providing a test compound; contacting the test compound with a cell that expresses an isolated HCV polyprotein or fragment thereof; and identifying the test compound as an inducer of an HCV replicase complex defect, wherein the compound is a compound of Formula (III)

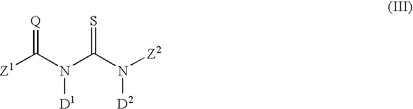

wherein
Q is oxygen or sulfur;
D¹ and D² are independently selected from the group consisting of hydrogen and methyl;
with the proviso that said compound of Formula (III) is not a compound of the formula (III-a):

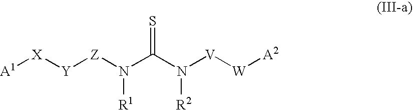

wherein

A₁ and A₂ are independently optionally substituted C₁-C₁₂ alkyl, optionally substituted mono- or di-(C₁-C₈ alkyl)amino, optionally substituted C₂-C₁₂ alkenyl, optionally substituted C₃-C₈ cycloalkyl, an optionally substituted partially unsaturated or aromatic carbocyclic group, or an optionally substituted saturated, partially unsaturated, or aromatic heterocyclic group, wherein at least one of A₁ and A₂ is an optionally substituted aromatic carbocyclic group or an optionally substituted aromatic heterocyclic group;

X and W are independently O, S, NR, or absent, wherein R is hydrogen, optionally substituted (C₁-C₆)alkyl, or optionally substituted aryl(C₀-C₄)alkyl;

V is (C₁-C₆)alkyl, (C₂-C₆)alkenyl, (C₃-C₇)cycloalkyl, or absent, wherein when V is absent, W is absent; and

Y is (C₁-C₆) alkyl, (C₁-C₆)alkyl substituted with (C₃-C₇)cycloalkyl, (C₂-C₆) alkenyl, (C₃-C₇)cycloalkyl, or absent; and

Z is carbonyl, thiocarbonyl, imino, or C₁-C₆ alkylimino; and

R₁ and R₂ are independently hydrogen or methyl;

and with the further proviso that said compound of Formula (III) is not a compound of the formula (III-b):

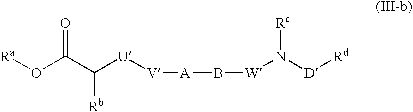

wherein
R^{a} is hydrogen, a substituted or unsubstituted alkyl or cycloalkyl group, a substituted or unsubstituted aryl group or a saturated or unsaturated, optionally substituted heterocyclic group;
R^{b} is hydrogen, a substituted or unsubstituted alkyl or cycloalkyl group, a substituted or unsubstituted aryl group, a saturated or unsaturated, optionally substituted heterocyclic group, an optionally substituted alkenyl group, an optionally substituted alkinyl group, —NR^{2′}SO₂R^{2″}, —NR^{2′}COOR^{2′}, —NR^{2′}COR^{2′}, —NR^{2′}CON(R^{2′})₂, or —N R^{2′}CSN(R^{2′})₂;
R^{2′} is hydrogen, a substituted or unsubstituted alkyl or cycloalkyl group, a substituted or unsubstituted aryl group or a saturated or unsaturated, optionally substituted heterocyclic group;
R^{2″} is a substituted or unsubstituted alkyl, alkenyl or cycloalkyl group, a substituted or unsubstituted aryl group, or a saturated or unsaturated, optionally substituted heterocyclic group;
U′ is a direct bond or a substituted or unsubstituted alkylene group;
V′ is a substituted or unsubstituted alkylene group, —NR^{2′}CO—, or —NR^{2′}SO₂—;
A and B are each independently an optionally substituted 1,3- or 1,4-bridging phenylene group or an optionally substituted 2,4- or 2,5-bridging thienylene group;
W′ is a direct bond or an optionally substituted alkylene group;
D′ is a direct bond or

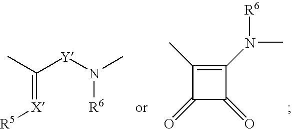

R^{c} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{d}, Y′, R⁵, or R⁶, if present, with formation of an optionally substituted heterocyclic ring system which includes the nitrogen atom to which R^{c} is bonded, and may be saturated or unsaturated and may contain further heteroatoms;
R^{d} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{c}, Y, R⁵ or R⁶, if present, with formation of an optionally substituted heterocyclic ring system which includes the nitrogen atom to which R^{d} is bonded and may be saturated or unsaturated and may contain further heteroatoms;

### X′ is CHNO₂, CHCN, O, N or S;

Y′ is a direct bond or an optionally substituted alkylene or alkine group;
R⁵ is absent, or is hydrogen, a substituted or unsubstituted alkyl or cycloalkyl group, —NO₂, —CN, —COR^{5′}, —COOR^{5′}, or is connected to one of R^{c}, Y′, R^{d}, or R⁶, if present, with formation of an optionally substituted carbocyclic or heterocyclic ring system which includes X′ and can be saturated or unsaturated and may optionally contain one or more additional heteroatoms;
R^{5′} is hydrogen, a substituted or unsubstituted alkyl or cycloalkyl group, a substituted or unsubstituted aryl group or a saturated or unsaturated, optionally substituted heterocyclic group which may optionally contain one or more additional heteroatoms;
R⁶ is a arylcarbonyl group, or a heteroarylcarbonyl group;
and wherein if A is a phenylene group and V′ is —NR^{2′}CO— or —NR^{2′} SO₂—, D′ is not a direct bond and X′ is not N;
and with the further proviso that said compound of Formula (III) is not a compound of the formula (III-c)

X^{1″}—Y^{1″}-Z^{1″}-W^{1″} (III-c)

wherein
X^{1″} is optionally substituted aryl, optionally substituted (C₁-C₈)alkyl-aryl, optionally substituted heteroaryl, or optionally substituted heteroarylalkyl;
Y^{1″} is —NR^{A}CSNR^{A}CO—, wherein R^{A} is independently hydrogen or lower alkyl;
Z^{1″} is optionally substituted phenylene, optionally substituted monocyclic heteroarylene, optionally substituted monocyclic non-aromatic heterocycle-diyl, or optionally substituted monocyclic cycloalkane-diyl;
W^{1″} is a group represented by the formula:

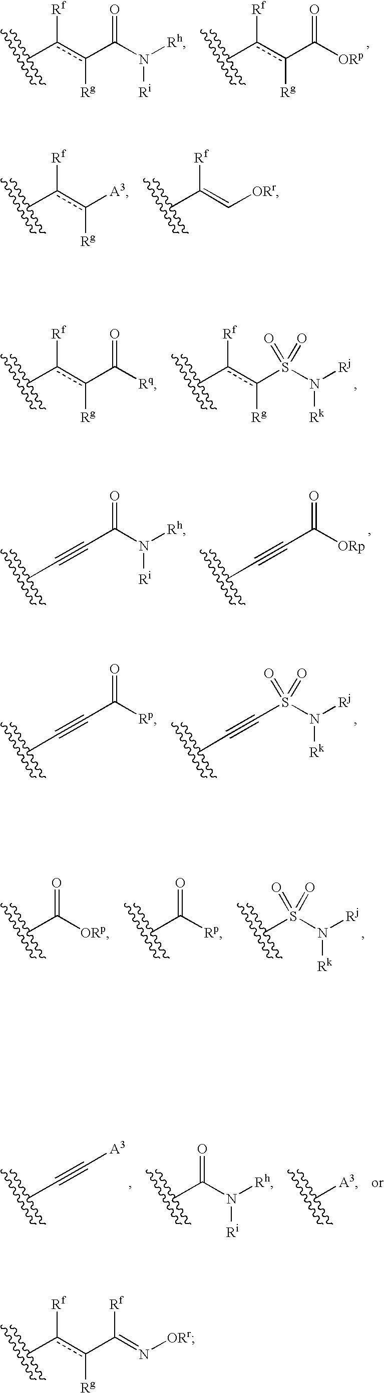

wherein
R^{f}, R^{g}, R^{h}, Rⁱ, R^{j}, and R^{k} are each independently hydrogen, halogen, optionally substituted lower alkyl, optionally substituted lower alkyloxy, optionally substituted lower alkylthio, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, optionally substituted non-aromatic heterocyclic group, or optionally substituted amino;
R^{p}, R^{q}, and R^{r} are each independently hydrogen, optionally substituted lower alkyl, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, or optionally substituted non-aromatic heterocyclic group;
A³ is an optionally substituted aryl or an optionally substituted heteroaryl group;
and with the further proviso that in said compound of Formula (III), Z¹ is not a 3-pyridyl group or a substituted 3-pyridyl group when Z² is —OH, —NH₂, —(C₁-C₆)alkylamino, di(C₁-C₆)alkylamino, an alkyl group, a substituted alkyl group, —(C₃-C₈) cycloalkyl group, a substituted —(C₃-C₈) cycloalkyl group, —(C₃-C₈) cycloalkyl-(C₁-C₆)alkyl group, —O-alkyl group, a substituted —O-alkyl group, —O—(C₃-C₈) cycloalkyl group, a substituted —O—(C₃-C₈) cycloalkyl group, a phenyl group, a fused-phenyl group, a phenyl group substituted with one or more independently selected halogen, —OCH₃, nitro, or dimethyl amino groups, a substituted fused-phenyl group substituted with one or more independently selected halogen, —OCH₃, nitro, or dimethyl amino groups, a —(C₁-C₈) alkyl-phenyl group, —O—(C₀-C₈) alkyl-phenyl group, —(C₃-C₆) alkylene group, —O—(C₃-C₆) alkylene group, —(C₃-C₆) alkynyl group, —O—(C₃-C₆) alkynyl group, —CO(C₁-C₆) alkyl group, —NHCO(C₁-C₆) alkyl group, —NHSO₂(C₁-C₆) alkyl group, —SO₂(C₁-C₆) alkyl group, or —SO₂(C₀-C₆)alkyl-phenyl group;
and with the further proviso that in said compound of Formula (III), Z¹ is not a furyl group or a substituted furyl group when D² and Z² are independently selected from the group consisting of hydrogen and methyl;
and with the further proviso that in said compound comprising structure (III), when D² is —CH₃ and Z² is an optionally substituted phenyl, Z¹ is not selected from the group consisting of —R³³, OR³⁴, and —NR³⁵R³⁶,
wherein
R³³ is hydrogen, optionally substituted (C₃-C₈)cycloalkyl, optionally substituted phenyl, optionally substituted heteroaryl or phenyl-(C₁-C₄) alkyl which is optionally substituted on the phenyl ring, (C₁-C₆)alkyl, (C₂-C₆)alkenyl, (C₂-C₆)alkynyl, the three latter radicals independently being optionally substituted with one or more radicals selected from the group consisting of halogen, (C₁-C₄)alkoxy, (C₁-C₄)alkylthio and NR³⁷R³⁸;
R³⁴ is (C₁-C₆)alkyl, (C₂-C₆)alkenyl, (C₂-C₆)alkynyl, the three latter radicals independently optionally substituted with one or more radicals selected from the group consisting of halogen, (C₁-C₄)alkoxy, (C₁-C₄)alkylthio and NR³⁷R³⁸, or R³⁴ is (C₃-C₆)cycloalkyl optionally substituted with one or more groups selected from the group consisting of halogen, (C₁-C₄)alkyl and (C₁-C₄)alkoxy, and (C₃-C₆)-cycloalkyl-(C₁-C₃)alkyl;
R³⁵ and R³⁶ are hydrogen, (C₁-C₆)alkyl, (C₂-C₆)alkenyl, (C₂-C₆)alkynyl, the 3 latter groups independently optionally substituted by one or more halogen radicals;
R³⁷ is independently selected from the group consisting of hydrogen, (C₁-C₄)alkyl, ((C₁-C₄)alkyl)carbonyl, ((C₁-C₄)alkoxy)carbonyl and CHO;
R³⁸ is independently selected from the group consisting of H and (C₁-C₄)alkyl;
and with the further proviso that in said compound of Formula (III), Z¹ and Z² are not both selected from the group consisting of hydrogen and alkyl;
and with the further proviso that in said compound of Formula (III), Z² is not hydrogen when D¹ and D² are both hydrogen;
and with the further proviso that in said compound of Formula (III), Z¹ is not (C₁-C₆) alkyl, (C₁-C₆) alkoxy, pyridyl, or aryl when D¹ and D² are both hydrogen and when Z² is selected from the group consisting of hydrogen, a (C₁-C₆) alkyl group optionally substituted with 1 to 3 substituents independently selected from the group consisting of hydroxyl, (C₁-C₆) alkoxyl, carboxyl, (C₁-C₆) alkoxycarbonyl, lower alkylthio, fluorine, chlorine, bromine, iodine, amino, mono- or di-substituted amino, phthalimido and nitrogen-containing heterocyclic groups, a (C₃-C₈) cycloalkyl group, a (C₂-C₆) alkenyl group, an arylalkyl group optionally substituted with from 1 to 5 substituents independently selected from the group consisting of (C₁-C₆) alkyl, hydroxyl, (C₁-C₆) alkoxy, halogen, nitro, and amino groups, an aryl group optionally substituted with from 1 to 5 independently selected substituents selected from the group consisting of (C₁-C₆) alkyl, hydroxyl, (C₁-C₆) alkoxy, halogen, nitro, and amino groups, a substituted or unsubstituted heterocyclic group, a (C₁-C₆) alkanoyl group, a benzoyl or a naphthoyl group either of which is optionally substituted with from 1 to 5 independently selected substituents selected from the group consisting of (C₁-C₆) alkyl, hydroxyl, (C₁-C₆) alkoxy, halogen, nitro, and amino groups, a pyridylcarbonyl group, a (C₁-C₆) alkoxycarbonyl group or an amino group optionally substituted with (C₁-C₆) alkyl, arylalkyl having 7 to 15 carbon atoms, substituted or unsubstituted aryl, (C₁-C₆) alkanoyl, optionally substituted aroyl, and (C₁-C₆) alkylidene groups;
and with the further proviso that in said compound of Formula (III), Z¹ is not —O—(CH₂)ₜ—CH₃, wherein t is 0-4 and Z² is halophenyl;
and with the further proviso that in said compound of Formula (III), Z¹ is not a substituted pyridyl, pyridinyl, pyrazinyl, pyridazinyl group substituted with —N(R²¹)-(optionally substituted phenyl), wherein said R²¹ is hydrogen, aryl, formyl, (C₁-C₆)alkylcarbonyl, (C₁-C₆)alkyl, (C₁-C₆)alkyloxycarbonyl, (C₁-C₆)alkyl substituted with formyl, (C₁-C₆)alkylcarbonyl, (C₁-C₆)alkyloxycarbonyl, (C₁-C₆)alkylcarbonyloxy, or (C₁-C₆)alkyloxy(C₁-C₆)alkylcarbonyl optionally substituted with (C₁-C₆)alkyloxycarbonyl;
and with the further proviso that in said compound of Formula (III), Z¹ is not a 3-(4-trifluoromethyl-pyridinyl) group or a 3-(4-trifluoromethyl-pyridinyl N-oxide) group;
and with the further proviso that in said compound of Formula (III), Z¹ is not a substituted or unsubstituted group selected from the group consisting of

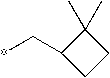

and a —(CH₂)ₜ-adamantanyl group, wherein t is 0-4;
and with the further proviso that in said compound of Formula (III), Z² is not a

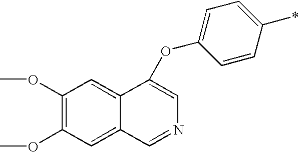

group, which is unsubstituted, or which is substituted with one or more substituents independently selected from the group consisting of halogens and alkyl groups;
and with the further proviso that in said compound of Formula (III), Z² is not a substituted or unsubstituted group selected from the group consisting of:

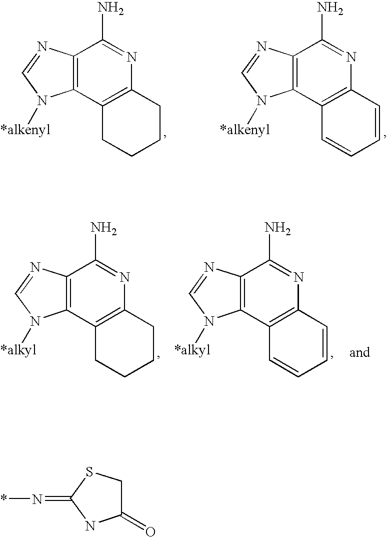

and with the further proviso that said compound of Formula (III), is not a compound set forth in Appendix A, Appendix B, or Appendix C.

In another embodiment, a compound of the present invention is a replicase complex defect inducer identified by the method of screening a test compound for replicase complex defect inducer activity comprising: providing a test compound; contacting the test compound with an HCV virion; and identifying the test compound as an inducer of an HCV replicase complex defect when the level of p14 protein is increased, wherein said compound is a compound of Formula (III)

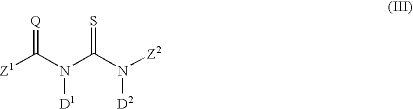

wherein
Q is oxygen or sulfur;
D¹ and D² are independently selected from the group consisting of hydrogen and methyl;
with the proviso that said compound of Formula (III) is not a compound of the formula (III-a):

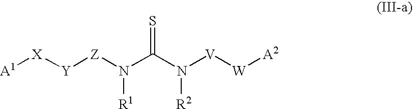

wherein

A₁ and A₂ are independently optionally substituted C₁-C₁₂ alkyl, optionally substituted mono- or di-(C₁-C₈ alkyl)amino, optionally substituted C₂-C₁₂ alkenyl, optionally substituted C₃-C₈ cycloalkyl, an optionally substituted partially unsaturated or aromatic carbocyclic group, or an optionally substituted saturated, partially unsaturated, or aromatic heterocyclic group, wherein at least one of A₁ and A₂ is an optionally substituted aromatic carbocyclic group or an optionally substituted aromatic heterocyclic group;

X and W are independently O, S, NR, or absent, wherein R is hydrogen, optionally substituted (C₁-C₆)alkyl, or optionally substituted aryl(C₀-C₄)alkyl;

V is (C₁-C₆)alkyl, (C₂-C₆)alkenyl, (C₃-C₇)cycloalkyl, or absent, wherein when V is absent, W is absent; and

Y is (C₁-C₆) alkyl, (C₁-C₆)alkyl substituted with (C₃-C₇)cycloalkyl, (C₂-C₆) alkenyl, (C₃-C₇)cycloalkyl, or absent; and

Z is carbonyl, thiocarbonyl, imino, or C₁-C₆ alkylimino; and

R₁ and R₂ are independently hydrogen or methyl;

and with the further proviso that said compound of Formula (III) is not a compound of the formula (III-b):

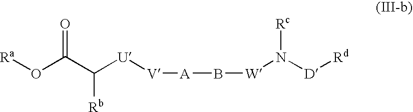

wherein
R^{a} is hydrogen, a substituted or unsubstituted alkyl or cycloalkyl group, a substituted or unsubstituted aryl group or a saturated or unsaturated, optionally substituted heterocyclic group;
R^{b} is hydrogen, a substituted or unsubstituted alkyl or cycloalkyl group, a substituted or unsubstituted aryl group, a saturated or unsaturated, optionally substituted heterocyclic group, an optionally substituted alkenyl group, an optionally substituted alkinyl group, —NR^{2′}SO₂R^{2′}, —NR^{2′}COOR^{2′}, —NR^{2′}COR^{2′}, —NR^{2′}CON(R^{2′})₂, or —N R^{2′}CSN(R^{2′})₂;
R^{2′} is hydrogen, a substituted or unsubstituted alkyl or cycloalkyl group, a substituted or unsubstituted aryl group or a saturated or unsaturated, optionally substituted heterocyclic group;
R^{2″} is a substituted or unsubstituted alkyl, alkenyl or cycloalkyl group, a substituted or unsubstituted aryl group, or a saturated or unsaturated, optionally substituted heterocyclic group;
U′ is a direct bond or a substituted or unsubstituted alkylene group;
V′ is a substituted or unsubstituted alkylene group, —NR^{2′}CO—, or —NR^{2′}SO₂—;
A and B are each independently an optionally substituted 1,3- or 1,4-bridging phenylene group or an optionally substituted 2,4- or 2,5-bridging thienylene group;
W′ is a direct bond or an optionally substituted alkylene group;
D′ is a direct bond or

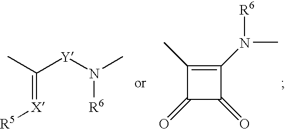

R^{c} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{d}, Y′, R⁵, or R⁶, if present, with formation of an optionally substituted heterocyclic ring system which includes the nitrogen atom to which R^{c} is bonded, and may be saturated or unsaturated and may contain further heteroatoms;
R^{d} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{c}, Y, R⁵ or R⁶, if present, with formation of an optionally substituted heterocyclic ring system which includes the nitrogen atom to which R^{d} is bonded and may be saturated or unsaturated and may contain further heteroatoms;

### X′ is CHNO₂, CHCN, O, N or S;

Y′ is a direct bond or an optionally substituted alkylene or alkine group;
R⁵ is absent, or is hydrogen, a substituted or unsubstituted alkyl or cycloalkyl group, —NO₂, —CN, —COR^{5′}, —COOR^{5′}, or is connected to one of R^{c}, Y′, R^{d}, or R⁶, if present, with formation of an optionally substituted carbocyclic or heterocyclic ring system which includes X′ and can be saturated or unsaturated and may optionally contain one or more additional heteroatoms;
R^{5′} is hydrogen, a substituted or unsubstituted alkyl or cycloalkyl group, a substituted or unsubstituted aryl group or a saturated or unsaturated, optionally substituted heterocyclic group which may optionally contain one or more additional heteroatoms;
R⁶ is a arylcarbonyl group, or a heteroarylcarbonyl group;
and wherein if A is a phenylene group and V′ is —NR^{2′}CO— or —NR^{1′}SO₂—, D′ is not a direct bond and X′ is not N;
and with the further proviso that said compound of Formula (III) is not a compound of the formula (III-c)

X^{1″}—Y^{1″}-Z^{1″}-W^{1″} (III-c)

wherein
X^{1″} is optionally substituted aryl, optionally substituted (C₁-C₈)alkyl-aryl, optionally substituted heteroaryl, or optionally substituted heteroarylalkyl;
Y^{1″} is —NR^{A}CSNR^{A}CO—, wherein R^{A} is independently hydrogen or lower alkyl;
Z^{1″} is optionally substituted phenylene, optionally substituted monocyclic heteroarylene, optionally substituted monocyclic non-aromatic heterocycle-diyl, or optionally substituted monocyclic cycloalkane-diyl;
W^{1″} is a group represented by the formula:

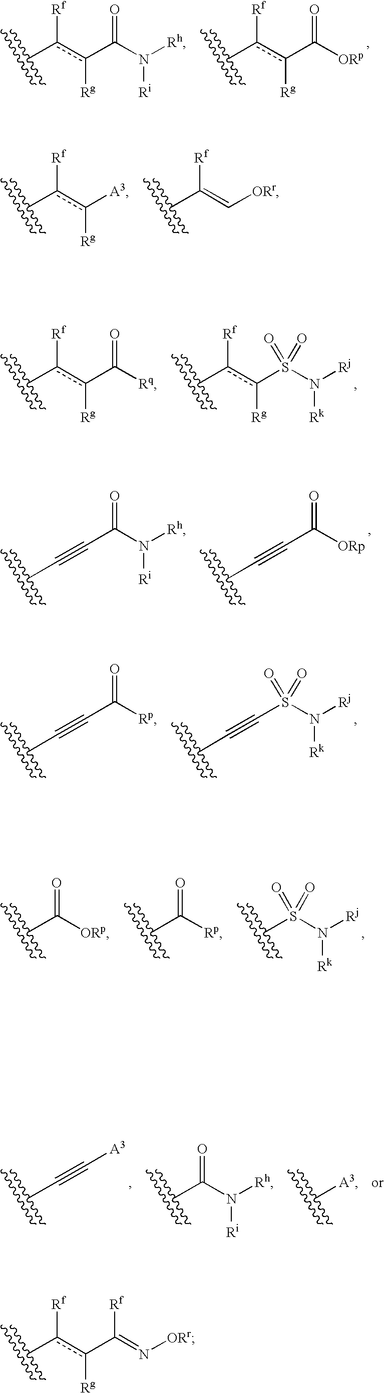

wherein
R^{f}, R^{g}, R^{h}, Rⁱ, R^{j}, and R^{k} are each independently hydrogen, halogen, optionally substituted lower alkyl, optionally substituted lower alkyloxy, optionally substituted lower alkylthio, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, optionally substituted non-aromatic heterocyclic group, or optionally substituted amino;
R^{p}, R^{q}, and R^{r} are each independently hydrogen, optionally substituted lower alkyl, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, or optionally substituted non-aromatic heterocyclic group;
A³ is an optionally substituted aryl or an optionally substituted heteroaryl group;
and with the further proviso that in said compound of Formula (III), Z¹ is not a 3-pyridyl group or a substituted 3-pyridyl group when Z² is —OH, —NH₂, —(C₁-C₆)alkylamino, di(C₁-C₆)alkylamino, an alkyl group, a substituted alkyl group, —(C₃-C₈) cycloalkyl group, a substituted —(C₃-C₈) cycloalkyl group, —(C₃-C₈) cycloalkyl-(C₁-C₆)alkyl group, —O-alkyl group, a substituted —O-alkyl group, —O—(C₃-C₈) cycloalkyl group, a substituted —O—(C₃-C₈) cycloalkyl group, a phenyl group, a fused-phenyl group, a phenyl group substituted with one or more independently selected halogen, —OCH₃, nitro, or dimethyl amino groups, a substituted fused-phenyl group substituted with one or more independently selected halogen, —OCH₃, nitro, or dimethyl amino groups, a —(C₁-C₈) alkyl-phenyl group, —O—(C₀-C₈) alkyl-phenyl group, —(C₃-C₆) alkylene group, —O—(C₃-C₆) alkylene group, —(C₃-C₆) alkynyl group, —O—(C₃-C₆) alkynyl group, —CO(C₁-C₆) alkyl group, —NHCO(C₁-C₆) alkyl group, —NHSO₂(C₁-C₆) alkyl group, —SO₂(C₁-C₆) alkyl group, or —SO₂(C₀-C₆)alkyl-phenyl group;
and with the further proviso that in said compound of Formula (III), Z¹ is not a furyl group or a substituted furyl group when D² and Z² are independently selected from the group consisting of hydrogen and methyl;
and with the further proviso that in said compound comprising structure (III), when D² is —CH₃ and Z² is an optionally substituted phenyl, Z¹ is not selected from the group consisting of —R³³, OR³⁴, and —NR³⁵R³⁶,
wherein
R³³ is hydrogen, optionally substituted (C₃-C₈)cycloalkyl, optionally substituted phenyl, optionally substituted heteroaryl or phenyl-(C₁-C₄) alkyl which is optionally substituted on the phenyl ring, (C₁-C₆)alkyl, (C₂-C₆)alkenyl, (C₂-C₆)alkynyl, the three latter radicals independently being optionally substituted with one or more radicals selected from the group consisting of halogen, (C₁-C₄)alkoxy, (C₁-C₄)alkylthio and NR³⁷R³⁸;
R³⁴ is (C₁-C₆)alkyl, (C₂-C₆)alkenyl, (C₂-C₆)alkynyl, the three latter radicals independently optionally substituted with one or more radicals selected from the group consisting of halogen, (C₁-C₄)alkoxy, (C₁-C₄)alkylthio and NR³⁷R³⁸, or R³⁴ is (C₃-C₆)cycloalkyl optionally substituted with one or more groups selected from the group consisting of halogen, (C₁-C₄)alkyl and (C₁-C₄)alkoxy, and (C₃-C₆)-cycloalkyl-(C₁-C₃)alkyl;
R³⁵ and R³⁶ are hydrogen, (C₁-C₆)alkyl, (C₂-C₆)alkenyl, (C₂-C₆)alkynyl, the 3 latter groups independently optionally substituted by one or more halogen radicals;
R³⁷ is independently selected from the group consisting of hydrogen, (C₁-C₄)alkyl, ((C₁-C₄)alkyl)carbonyl, ((C₁-C₄)alkoxy)carbonyl and CHO;
R³⁸ is independently selected from the group consisting of H and (C₁-C₄)alkyl;
and with the further proviso that in said compound of Formula (III), Z¹ and Z² are not both selected from the group consisting of hydrogen and alkyl;
and with the further proviso that in said compound of Formula (III), Z² is not hydrogen when D¹ and D² are both hydrogen;
and with the further proviso that in said compound of Formula (III), Z¹ is not (C₁-C₆) alkyl, (C₁-C₆) alkoxy, pyridyl, or aryl when D¹ and D² are both hydrogen and when Z² is selected from the group consisting of hydrogen, a (C₁-C₆) alkyl group optionally substituted with 1 to 3 substituents independently selected from the group consisting of hydroxyl, (C₁-C₆) alkoxyl, carboxyl, (C₁-C₆) alkoxycarbonyl, lower alkylthio, fluorine, chlorine, bromine, iodine, amino, mono- or di-substituted amino, phthalimido and nitrogen-containing heterocyclic groups, a (C₃-C₈) cycloalkyl group, a (C₂-C₆) alkenyl group, an arylalkyl group optionally substituted with from 1 to 5 substituents independently selected from the group consisting of (C₁-C₆) alkyl, hydroxyl, (C₁-C₆) alkoxy, halogen, nitro, and amino groups, an aryl group optionally substituted with from 1 to 5 independently selected substituents selected from the group consisting of (C₁-C₆) alkyl, hydroxyl, (C₁-C₆) alkoxy, halogen, nitro, and amino groups, a substituted or unsubstituted heterocyclic group, a (C₁-C₆) alkanoyl group, a benzoyl or a naphthoyl group either of which is optionally substituted with from 1 to 5 independently selected substituents selected from the group consisting of (C₁-C₆) alkyl, hydroxyl, (C₁-C₆) alkoxy, halogen, nitro, and amino groups, a pyridylcarbonyl group, a (C₁-C₆) alkoxycarbonyl group or an amino group optionally substituted with (C₁-C₆) alkyl, arylalkyl having 7 to 15 carbon atoms, substituted or unsubstituted aryl, (C₁-C₆) alkanoyl, optionally substituted aroyl, and (C₁-C₆) alkylidene groups;
and with the further proviso that in said compound of Formula (III), Z¹ is not —O—(CH₂)ₜ—CH₃, wherein t is 0-4 and Z² is halophenyl;
and with the further proviso that in said compound of Formula (III), Z¹ is not a substituted pyridyl, pyrimidinyl, pyrazinyl, pyridazinyl group substituted with —N(R²¹)-(optionally substituted phenyl), wherein said R²¹ is hydrogen, aryl, formyl, (C₁-C₆)alkylcarbonyl, (C₁-C₆)alkyl, (C₁-C₆)alkyloxycarbonyl, (C₁-C₆)alkyl substituted with formyl, (C₁-C₆)alkylcarbonyl, (C₁-C₆)alkyloxycarbonyl, (C₁-C₆)alkylcarbonyloxy, or (C₁-C₆)alkyloxy(C₁-C₆)alkylcarbonyl optionally substituted with (C₁-C₆)alkyloxycarbonyl;
and with the further proviso that in said compound of Formula (III), Z¹ is not a 3-(4-trifluoromethyl-pyridinyl) group or a 3-(4-trifluoromethyl-pyridinyl N-oxide) group;
and with the further proviso that in said compound of Formula (III), Z¹ is not a substituted or unsubstituted group selected from the group consisting of

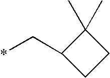

and a —(CH₂)ₜ-adamantanyl group, wherein t is 0-4;
and with the further proviso that in said compound of Formula (III), Z² is not a

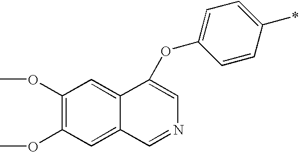

group, which is unsubstituted, or which is substituted with one or more substituents independently selected from the group consisting of halogens and alkyl groups;
and with the further proviso that in said compound of Formula (III), Z² is not a substituted or unsubstituted group selected from the group consisting of:

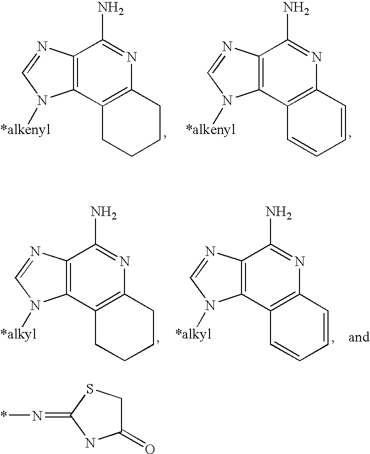

and with the further proviso that said compound of Formula (III), is not a compound set forth in Appendix A, Appendix B, or Appendix C.

In another embodiment, a compound of the present invention is a replicase complex defect inducer identified by the method of screening a test compound for replicase complex defect inducer activity comprising: providing a test compound; contacting the test compound with an HCV replicon; and identifying the test compound as an inducer of an HCV replicase complex defect when the level of p14 protein is increased, wherein said compound is a compound of Formula (III)

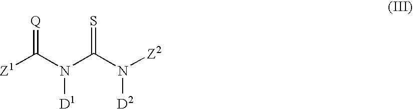

wherein
Q is oxygen or sulfur;
D¹ and D² are independently selected from the group consisting of hydrogen and methyl;
with the proviso that said compound of Formula (III) is not a compound of the formula (III-a):

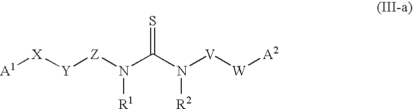

wherein

A₁ and A₂ are independently optionally substituted C₁-C₁₂ alkyl, optionally substituted mono- or di-(C₁-C₈ alkyl)amino, optionally substituted C₂-C₁₂ alkenyl, optionally substituted C₃-C₈ cycloalkyl, an optionally substituted partially unsaturated or aromatic carbocyclic group, or an optionally substituted saturated, partially unsaturated, or aromatic heterocyclic group, wherein at least one of A₁ and A₂ is an optionally substituted aromatic carbocyclic group or an optionally substituted aromatic heterocyclic group;

X and W are independently O, S, NR, or absent, wherein R is hydrogen, optionally substituted (C₁-C₆)alkyl, or optionally substituted aryl(C₀-C₄)alkyl;

V is (C₁-C₆)alkyl, (C₂-C₆)alkenyl, (C₃-C₇)cycloalkyl, or absent, wherein when V is absent, W is absent; and

Y is (C₁-C₆) alkyl, (C₁-C₆)alkyl substituted with (C₃-C₇)cycloalkyl, (C₂-C₆) alkenyl, (C₃-C₇)cycloalkyl, or absent; and

Z is carbonyl, thiocarbonyl, imino, or C₁-C₆ alkylimino; and

R₁ and R₂ are independently hydrogen or methyl;

and with the further proviso that said compound of Formula (III) is not a compound of the formula (III-b):

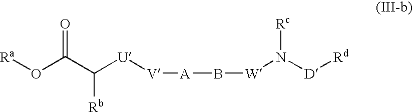

wherein
R^{a} is hydrogen, a substituted or unsubstituted alkyl or cycloalkyl group, a substituted or unsubstituted aryl group or a saturated or unsaturated, optionally substituted heterocyclic group;
R^{b} is hydrogen, a substituted or unsubstituted alkyl or cycloalkyl group, a substituted or unsubstituted aryl group, a saturated or unsaturated, optionally substituted heterocyclic group, an optionally substituted alkenyl group, an optionally substituted alkinyl group, —NR^{2′}SO₂R^{2″}, —NR^{2′}COOR^{2′}, —NR^{2′}COR^{2′}, —NR^{2′}CON(R^{2′})₂, or —N R^{2′}CSN(R^{2′})₂;
R^{2′} is hydrogen, a substituted or unsubstituted alkyl or cycloalkyl group, a substituted or unsubstituted aryl group or a saturated or unsaturated, optionally substituted heterocyclic group;
R^{2″} is a substituted or unsubstituted alkyl, alkenyl or cycloalkyl group, a substituted or unsubstituted aryl group, or a saturated or unsaturated, optionally substituted heterocyclic group;
U′ is a direct bond or a substituted or unsubstituted alkylene group;
V′ is a substituted or unsubstituted alkylene group, —NR^{2′}CO—, or —NR^{2′}SO₂—;
A and B are each independently an optionally substituted 1,3- or 1,4-bridging phenylene group or an optionally substituted 2,4- or 2,5-bridging thienylene group;
W′ is a direct bond or an optionally substituted alkylene group;
D′ is a direct bond or

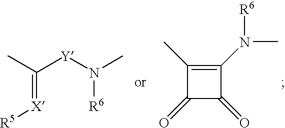

R^{c} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{d}, Y′, R⁵, or R⁶, if present, with formation of an optionally substituted heterocyclic ring system which includes the nitrogen atom to which R^{c} is bonded, and may be saturated or unsaturated and may contain further heteroatoms;
R^{d} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{c}, Y, R⁵ or R⁶, if present, with formation of an optionally substituted heterocyclic ring system which includes the nitrogen atom to which R^{d} is bonded and may be saturated or unsaturated and may contain further heteroatoms;

### X′ is CHNO₂, CHCN, O, N or S;

Y′ is a direct bond or an optionally substituted alkylene or alkine group;
R⁵ is absent, or is hydrogen, a substituted or unsubstituted alkyl or cycloalkyl group, —NO₂, —CN, —COR^{5′}, —COOR^{5′}, or is connected to one of R^{c}, Y′, R^{d}, or R⁶, if present, with formation of an optionally substituted carbocyclic or heterocyclic ring system which includes X′ and can be saturated or unsaturated and may optionally contain one or more additional heteroatoms;
R^{5′} is hydrogen, a substituted or unsubstituted alkyl or cycloalkyl group, a substituted or unsubstituted aryl group or a saturated or unsaturated, optionally substituted heterocyclic group which may optionally contain one or more additional heteroatoms;
R⁶ is a arylcarbonyl group, or a heteroarylcarbonyl group;
and wherein if A is a phenylene group and V′ is —NR^{2′}CO— or —NR^{2′} SO₂—, D′ is not a direct bond and X′ is not N;
and with the further proviso that said compound of Formula (III) is not a compound of the formula (III-c)

X^{1″}—Y^{1″}-Z^{1″}-W^{1″} (III-c)

wherein
X^{1″} is optionally substituted aryl, optionally substituted (C₁-C₈)alkyl-aryl, optionally substituted heteroaryl, or optionally substituted heteroarylalkyl;
Y^{1″} is —NR^{A}CSNR^{A}CO—, wherein R^{A} is independently hydrogen or lower alkyl;
Z^{1″} is optionally substituted phenylene, optionally substituted monocyclic heteroarylene, optionally substituted monocyclic non-aromatic heterocycle-diyl, or optionally substituted monocyclic cycloalkane-diyl;
W^{1″} is a group represented by the formula:

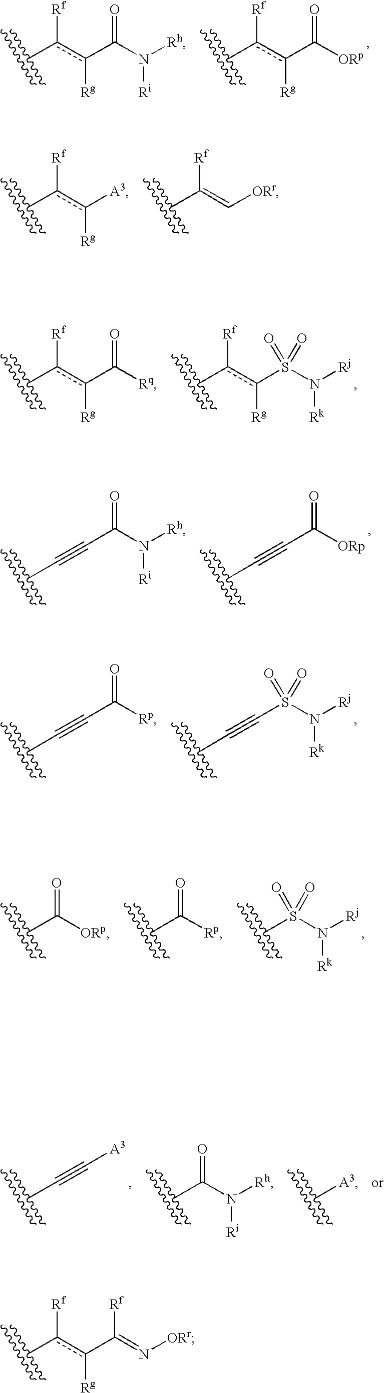

wherein
R^{f}, R^{g}, R^{h}, Rⁱ, R^{j}, and R^{k} are each independently hydrogen, halogen, optionally substituted lower alkyl, optionally substituted lower alkyloxy, optionally substituted lower alkylthio, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, optionally substituted non-aromatic heterocyclic group, or optionally substituted amino;
R^{p}, R^{q}, and R^{r} are each independently hydrogen, optionally substituted lower alkyl, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, or optionally substituted non-aromatic heterocyclic group;
A³ is an optionally substituted aryl or an optionally substituted heteroaryl group;
and with the further proviso that in said compound of Formula (III), Z¹ is not a 3-pyridyl group or a substituted 3-pyridyl group when Z² is —OH, —NH₂, —(C₁-C₆)alkylamino, di(C₁-C₆)alkylamino, an alkyl group, a substituted alkyl group, —(C₃-C₈) cycloalkyl group, a substituted —(C₃-C₈) cycloalkyl group, —(C₃-C₈) cycloalkyl-(C₁-C₆)alkyl group, —O-alkyl group, a substituted —O-alkyl group, —(C₃-C₈) cycloalkyl group, a substituted —O—(C₃-C₈) cycloalkyl group, a phenyl group, a fused-phenyl group, a phenyl group substituted with one or more independently selected halogen, —OCH₃, nitro, or dimethyl amino groups, a substituted fused-phenyl group substituted with one or more independently selected halogen, —OCH₃, nitro, or dimethyl amino groups, a —(C₁-C₈) alkyl-phenyl group, —O—(C₀-C₈) alkyl-phenyl group, —(C₃-C₆) alkylene group, —O—(C₃-C₆) alkylene group, —(C₃-C₆) alkynyl group, —O—(C₃-C₆) alkynyl group, —CO(C₁-C₆) alkyl group, —NHCO(C₁-C₆) alkyl group, —NHSO₂(C₁-C₆) alkyl group, —SO₂(C₁-C₆) alkyl group, or —SO₂(C₀-C₆)alkyl-phenyl group;
and with the further proviso that in said compound of Formula (III), Z¹ is not a furyl group or a substituted furyl group when D² and Z² are independently selected from the group consisting of hydrogen and methyl;
and with the further proviso that in said compound comprising structure (III), when D² is —CH₃ and Z² is an optionally substituted phenyl, Z¹ is not selected from the group consisting of —R³³, OR³⁴, and —NR³⁵R³⁶,
wherein
R³³ is hydrogen, optionally substituted (C₃-C₈)cycloalkyl, optionally substituted phenyl, optionally substituted heteroaryl or phenyl-(C₁-C₄) alkyl which is optionally substituted on the phenyl ring, (C₁-C₆)alkyl, (C₂-C₆)alkenyl, (C₂-C₆)alkynyl, the three latter radicals independently being optionally substituted with one or more radicals selected from the group consisting of halogen, (C₁-C₄)alkoxy, (C₁-C₄)alkylthio and NR³⁷R³⁸;
R³⁴ is (C₁-C₆)alkyl, (C₂-C₆)alkenyl, (C₂-C₆)alkynyl, the three latter radicals independently optionally substituted with one or more radicals selected from the group consisting of halogen, (C₁-C₄)alkoxy, (C₁-C₄)alkylthio and NR³⁷R³⁸, or R³⁴ is (C₃-C₆)cycloalkyl optionally substituted with one or more groups selected from the group consisting of halogen, (C₁-C₄)alkyl and (C₁-C₄)alkoxy, and (C₃-C₆)-cycloalkyl-(C₁-C₃)alkyl;
R³⁵ and R³⁶ are hydrogen, (C₁-C₆)alkyl, (C₂-C₆)alkenyl, (C₂-C₆)alkynyl, the 3 latter groups independently optionally substituted by one or more halogen radicals;
R³⁷ is independently selected from the group consisting of hydrogen, (C₁-C₄)alkyl, ((C₁-C₄)alkyl)carbonyl, ((C₁-C₄)alkoxy)carbonyl and CHO;
R³⁸ is independently selected from the group consisting of H and (C₁-C₄)alkyl;
and with the further proviso that in said compound of Formula (III), Z¹ and Z² are not both selected from the group consisting of hydrogen and alkyl;
and with the further proviso that in said compound of Formula (III), Z² is not hydrogen when D¹ and D² are both hydrogen;
and with the further proviso that in said compound of Formula (III), Z¹ is not (C₁-C₆) alkyl, (C₁-C₆) alkoxy, pyridyl, or aryl when D¹ and D² are both hydrogen and when Z² is selected from the group consisting of hydrogen, a (C₁-C₆) alkyl group optionally substituted with 1 to 3 substituents independently selected from the group consisting of hydroxyl, (C₁-C₆) alkoxyl, carboxyl, (C₁-C₆) alkoxycarbonyl, lower alkylthio, fluorine, chlorine, bromine, iodine, amino, mono- or di-substituted amino, phthalimido and nitrogen-containing heterocyclic groups, a (C₃-C₈) cycloalkyl group, a (C₂-C₆) alkenyl group, an arylalkyl group optionally substituted with from 1 to 5 substituents independently selected from the group consisting of (C₁-C₆) alkyl, hydroxyl, (C₁-C₆) alkoxy, halogen, nitro, and amino groups, an aryl group optionally substituted with from 1 to 5 independently selected substituents selected from the group consisting of (C₁-C₆) alkyl, hydroxyl, (C₁-C₆) alkoxy, halogen, nitro, and amino groups, a substituted or unsubstituted heterocyclic group, a (C₁-C₆) alkanoyl group, a benzoyl or a naphthoyl group either of which is optionally substituted with from 1 to 5 independently selected substituents selected from the group consisting of (C₁-C₆) alkyl, hydroxyl, (C₁-C₆) alkoxy, halogen, nitro, and amino groups, a pyridylcarbonyl group, a (C₁-C₆) alkoxycarbonyl group or an amino group optionally substituted with (C₁-C₆) alkyl, arylalkyl having 7 to 15 carbon atoms, substituted or unsubstituted aryl, (C₁-C₆) alkanoyl, optionally substituted aroyl, and (C₁-C₆) alkylidene groups;
and with the further proviso that in said compound of Formula (III), Z¹ is not O—(CH₂)ₜ—CH₃, wherein t is 0-4 and Z² is halophenyl;
and with the further proviso that in said compound of Formula (III), Z¹ is not a substituted pyridyl, pyrimidinyl, pyrazinyl, pyridazinyl group substituted with —N(R^{2′})-(optionally substituted phenyl), wherein said R^{2′} is hydrogen, aryl, formyl, (C₁-C₆)alkylcarbonyl, (C₁-C₆)alkyl, (C₁-C₆)alkyloxycarbonyl, (C₁-C₆)alkyl substituted with formyl, (C₁-C₆)alkylcarbonyl, (C₁-C₆)alkyloxycarbonyl, (C₁-C₆)alkylcarbonyloxy, or (C₁-C₆)alkyloxy(C₁-C₆)alkylcarbonyl optionally substituted with (C₁-C₆)alkyloxycarbonyl;
and with the further proviso that in said compound of Formula (III), Z¹ is not a 3-(4-trifluoromethyl-pyridinyl) group or a 3-(4-trifluoromethyl-pyridinyl N-oxide) group;
and with the further proviso that in said compound of Formula (III), Z¹ is not a substituted or unsubstituted group selected from the group consisting of

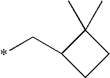

and a —(CH₂)ₜ-adamantanyl group, wherein t is 0-4;
and with the further proviso that in said compound of Formula (III), Z² is not a

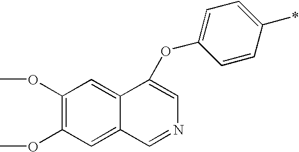

group, which is unsubstituted, or which is substituted with one or more substituents independently selected from the group consisting of halogens and alkyl groups;
and with the further proviso that in said compound of Formula (III), Z² is not a substituted or unsubstituted group selected from the group consisting of:

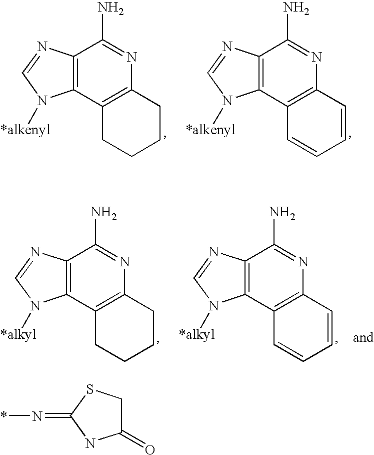

and with the further proviso that said compound of Formula (III), is not a compound set forth in Appendix A, Appendix B, or Appendix C.

In another embodiment, a compound of the present invention is a replicase complex defect inducer identified by the method of screening a test compound for replicase complex defect inducer activity comprising: providing a test compound; contacting the test compound with an isolated HCV replicase complex; and identifying the test compound as an inducer of an HCV replicase complex defect when the level of p14 protein is increased, wherein said compound is a compound of Formula (III)

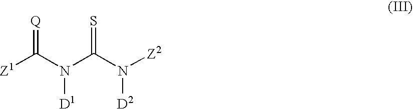

wherein
Q is oxygen or sulfur;
D¹ and D² are independently selected from the group consisting of hydrogen and methyl;
with the proviso that said compound of Formula (III) is not a compound of the formula (III-a):

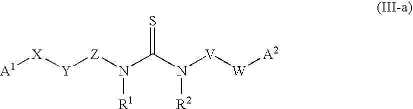

wherein

A₁ and A₂ are independently optionally substituted C₁-C₁₂ alkyl, optionally substituted mono- or di-(C₁-C₈ alkyl)amino, optionally substituted C₂-C₁₂ alkenyl, optionally substituted C₃-C₈ cycloalkyl, an optionally substituted partially unsaturated or aromatic carbocyclic group, or an optionally substituted saturated, partially unsaturated, or aromatic heterocyclic group, wherein at least one of A₁ and A₂ is an optionally substituted aromatic carbocyclic group or an optionally substituted aromatic heterocyclic group;

X and W are independently O, S, NR, or absent, wherein R is hydrogen, optionally substituted (C₁-C₆)alkyl, or optionally substituted aryl(C₀-C₄)alkyl;

V is (C₁-C₆)alkyl, (C₂-C₆)alkenyl, (C₃-C₇)cycloalkyl, or absent, wherein when V is absent, W is absent; and

Y is (C₁-C₆) alkyl, (C₁-C₆)alkyl substituted with (C₃-C₇)cycloalkyl, (C₂-C₆) alkenyl, (C₃-C₇)cycloalkyl, or absent; and

Z is carbonyl, thiocarbonyl, imino, or C₁-C₆ alkylimino; and

R₁ and R₂ are independently hydrogen or methyl;

and with the further proviso that said compound of Formula (III) is not a compound of the formula (III-b):

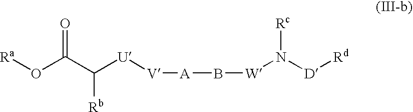

wherein
R^{a} is hydrogen, a substituted or unsubstituted alkyl or cycloalkyl group, a substituted or unsubstituted aryl group or a saturated or unsaturated, optionally substituted heterocyclic group;
R^{b} is hydrogen, a substituted or unsubstituted alkyl or cycloalkyl group, a substituted or unsubstituted aryl group, a saturated or unsaturated, optionally substituted heterocyclic group, an optionally substituted alkenyl group, an optionally substituted alkinyl group, —NR^{2′}SO₂R^{2″}, —NR^{2′}COOR^{2′}, —NR^{2′}COR^{2′}, —NR^{2′}CON(R^{2′})₂, or —N R^{2′}CSN(R^{2′})₂;
R^{2′} is hydrogen, a substituted or unsubstituted alkyl or cycloalkyl group, a substituted or unsubstituted aryl group or a saturated or unsaturated, optionally substituted heterocyclic group;
R^{2″} is a substituted or unsubstituted alkyl, alkenyl or cycloalkyl group, a substituted or unsubstituted aryl group, or a saturated or unsaturated, optionally substituted heterocyclic group;
U′ is a direct bond or a substituted or unsubstituted alkylene group;
V′ is a substituted or unsubstituted alkylene group, —NR^{2′}CO—, or —NR^{2′}SO₂—;
A and B are each independently an optionally substituted 1,3- or 1,4-bridging phenylene group or an optionally substituted 2,4- or 2,5-bridging thienylene group;
W′ is a direct bond or an optionally substituted alkylene group;
D′ is a direct bond or

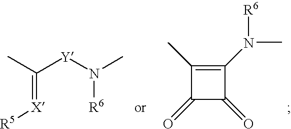

R^{c} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{d}, Y′, R⁵, or R⁶, if present, with formation of an optionally substituted heterocyclic ring system which includes the nitrogen atom to which R^{c} is bonded, and may be saturated or unsaturated and may contain further heteroatoms;
R^{d} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{c}, Y, R⁵ or R⁶, if present, with formation of an optionally substituted heterocyclic ring system which includes the nitrogen atom to which R^{d} is bonded and may be saturated or unsaturated and may contain further heteroatoms;

### X′ is CHNO₂, CHCN, O, N or S;

Y′ is a direct bond or an optionally substituted alkylene or alkine group;
R⁵ is absent, or is hydrogen, a substituted or unsubstituted alkyl or cycloalkyl group, —NO₂, —CN, —COR^{5′}, —COOR^{5′}, or is connected to one of R^{c}, Y′, R^{d}, or R⁶, if present, with formation of an optionally substituted carbocyclic or heterocyclic ring system which includes X′ and can be saturated or unsaturated and may optionally contain one or more additional heteroatoms;
R^{5′} is hydrogen, a substituted or unsubstituted alkyl or cycloalkyl group, a substituted or unsubstituted aryl group or a saturated or unsaturated, optionally substituted heterocyclic group which may optionally contain one or more additional heteroatoms;
R⁶ is a arylcarbonyl group, or a heteroarylcarbonyl group;
and wherein if A is a phenylene group and V′ is —NR^{2′}CO— or —NR^{2′}SO₂—, D′ is not a direct bond and X′ is not N;
and with the further proviso that said compound of Formula (III) is not a compound of the formula (III-c)

X^{1″}—Y^{1″}-Z^{1″}-W^{1″} (III-c)

wherein
X^{1″} is optionally substituted aryl, optionally substituted (C₁-C₈)alkyl-aryl, optionally substituted heteroaryl, or optionally substituted heteroarylalkyl;
Y^{1″} is —NR^{A}CSNR^{A}CO—, wherein R^{A} is independently hydrogen or lower alkyl;
Z^{1″} is optionally substituted phenylene, optionally substituted monocyclic heteroarylene, optionally substituted monocyclic non-aromatic heterocycle-diyl, or optionally substituted monocyclic cycloalkane-diyl;
W^{1″} is a group represented by the formula:

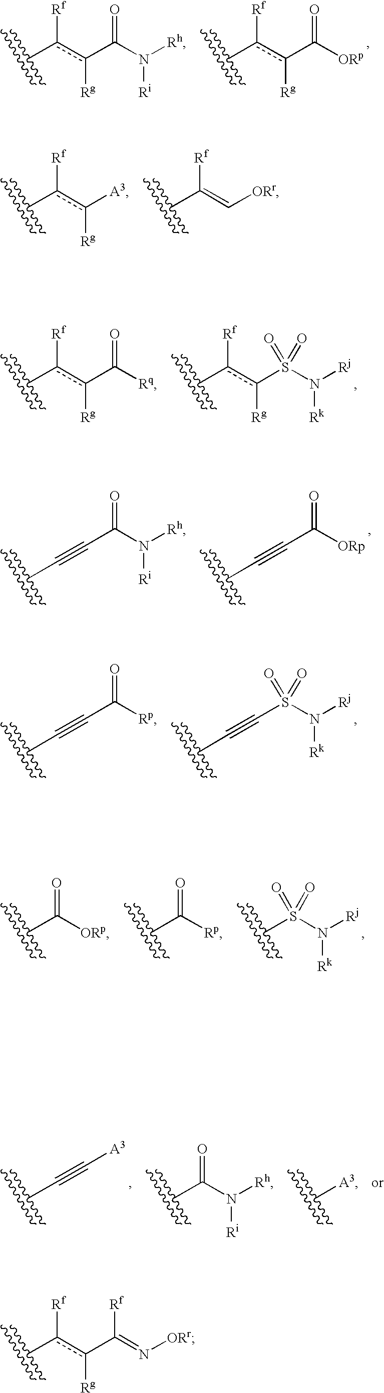

wherein
R^{f}, R^{g}, R^{h}, Rⁱ, R^{j}, and R^{k} are each independently hydrogen, halogen, optionally substituted lower alkyl, optionally substituted lower alkyloxy, optionally substituted lower alkylthio, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, optionally substituted non-aromatic heterocyclic group, or optionally substituted amino;
R^{p}, R^{q}, and R^{r} are each independently hydrogen, optionally substituted lower alkyl, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, or optionally substituted non-aromatic heterocyclic group;
A³ is an optionally substituted aryl or an optionally substituted heteroaryl group;
and with the further proviso that in said compound of Formula (III), Z¹ is not a 3-pyridyl group or a substituted 3-pyridyl group when Z² is —OH, —NH₂, —(C₁-C₆)alkylamino, di(C₁-C₆)alkylamino, an alkyl group, a substituted alkyl group, —(C₃-C₈) cycloalkyl group, a substituted —(C₃-C₈) cycloalkyl group, —(C₃-C₈) cycloalkyl-(C₁-C₆)alkyl group, —O-alkyl group, a substituted —O-alkyl group, —O—(C₃-C₈) cycloalkyl group, a substituted —O—(C₃-C₈) cycloalkyl group, a phenyl group, a fused-phenyl group, a phenyl group substituted with one or more independently selected halogen, —OCH₃, nitro, or dimethyl amino groups, a substituted fused-phenyl group substituted with one or more independently selected halogen, —OCH₃, nitro, or dimethyl amino groups, a —(C₁-C₈) alkyl-phenyl group, —O—(C₀-C₈) alkyl-phenyl group, —(C₃-C₆) alkylene group, —O—(C₃-C₆) alkylene group, —(C₃-C₆) alkynyl group, —O—(C₃-C₆) alkynyl group, —CO(C₁-C₆) alkyl group, —NHCO(C₁-C₆) alkyl group, —NHSO₂(C₁-C₆) alkyl group, —SO₂(C₁-C₆) alkyl group, or —SO₂(C₀-C₆)alkyl-phenyl group;
and with the further proviso that in said compound of Formula (III), Z¹ is not a furyl group or a substituted furyl group when D² and Z² are independently selected from the group consisting of hydrogen and methyl;
and with the further proviso that in said compound comprising structure (III), when D² is —CH₃ and Z² is an optionally substituted phenyl, Z¹ is not selected from the group consisting of —R³³, OR³⁴, and —NR³⁵R³⁶,
wherein
R³³ is hydrogen, optionally substituted (C₃-C₈)cycloalkyl, optionally substituted phenyl, optionally substituted heteroaryl or phenyl-(C₁-C₄) alkyl which is optionally substituted on the phenyl ring, (C₁-C₆)alkyl, (C₂-C₆)alkenyl, (C₂-C₆)alkynyl, the three latter radicals independently being optionally substituted with one or more radicals selected from the group consisting of halogen, (C₁-C₄)alkoxy, (C₁-C₄)alkylthio and NR³⁷R³⁸;
R³⁴ is (C₁-C₆)alkyl, (C₂-C₆)alkenyl, (C₂-C₆)alkynyl, the three latter radicals independently optionally substituted with one or more radicals selected from the group consisting of halogen, (C₁-C₄)alkoxy, (C₁-C₄)alkylthio and NR³⁷R³⁸, or R³⁴ is (C₃-C₆)cycloalkyl optionally substituted with one or more groups selected from the group consisting of halogen, (C₁-C₄)alkyl and (C₁-C₄)alkoxy, and (C₃-C₆)-cycloalkyl-(C₁-C₃)alkyl;
R³⁵ and R³⁶ are hydrogen, (C₁-C₆)alkyl, (C₂-C₆)alkenyl, (C₂-C₆)alkynyl, the 3 latter groups independently optionally substituted by one or more halogen radicals;
R³⁷ is independently selected from the group consisting of hydrogen, (C₁-C₄)alkyl, ((C₁-C₄)alkyl)carbonyl, ((C₁-C₄)alkoxy)carbonyl and CHO;
R³⁸ is independently selected from the group consisting of H and (C₁-C₄)alkyl;
and with the further proviso that in said compound of Formula (III), Z¹ and Z² are not both selected from the group consisting of hydrogen and alkyl;
and with the further proviso that in said compound of Formula (III), Z² is not hydrogen when D¹ and D² are both hydrogen;
and with the further proviso that in said compound of Formula (III), Z¹ is not (C₁-C₆) alkyl, (C₁-C₆) alkoxy, pyridyl, or aryl when D¹ and D² are both hydrogen and when Z² is selected from the group consisting of hydrogen, a (C₁-C₆) alkyl group optionally substituted with 1 to 3 substituents independently selected from the group consisting of hydroxyl, (C₁-C₆) alkoxyl, carboxyl, (C₁-C₆) alkoxycarbonyl, lower alkylthio, fluorine, chlorine, bromine, iodine, amino, mono- or di-substituted amino, phthalimido and nitrogen-containing heterocyclic groups, a (C₃-C₈) cycloalkyl group, a (C₂-C₆) alkenyl group, an arylalkyl group optionally substituted with from 1 to 5 substituents independently selected from the group consisting of (C₁-C₆) alkyl, hydroxyl, (C₁-C₆) alkoxy, halogen, nitro, and amino groups, an aryl group optionally substituted with from 1 to 5 independently selected substituents selected from the group consisting of (C₁-C₆) alkyl, hydroxyl, (C₁-C₆) alkoxy, halogen, nitro, and amino groups, a substituted or unsubstituted heterocyclic group, a (C₁-C₆) alkanoyl group, a benzoyl or a naphthoyl group either of which is optionally substituted with from 1 to 5 independently selected substituents selected from the group consisting of (C₁-C₆) alkyl, hydroxyl, (C₁-C₆) alkoxy, halogen, nitro, and amino groups, a pyridylcarbonyl group, a (C₁-C₆) alkoxycarbonyl group or an amino group optionally substituted with (C₁-C₆) alkyl, arylalkyl having 7 to 15 carbon atoms, substituted or unsubstituted aryl, (C₁-C₆) alkanoyl, optionally substituted aroyl, and (C₁-C₆) alkylidene groups;
and with the further proviso that in said compound of Formula (III), Z¹ is not —O—(CH₂)ₜ—CH₃, wherein t is 0-4 and Z² is halophenyl;
and with the further proviso that in said compound of Formula (III), Z¹ is not a substituted pyridyl, pyrimidinyl, pyrazinyl, pyridazinyl group substituted with —N(R²¹)-(optionally substituted phenyl), wherein said R²¹ is hydrogen, aryl, formyl, (C₁-C₆)alkylcarbonyl, (C₁-C₆)alkyl, (C₁-C₆)alkyloxycarbonyl, (C₁-C₆)alkyl substituted with formyl, (C₁-C₆)alkylcarbonyl, (C₁-C₆)alkyloxycarbonyl, (C₁-C₆)alkylcarbonyloxy, or (C₁-C₆)alkyloxy(C₁-C₆)alkylcarbonyl optionally substituted with (C₁-C₆)alkyloxycarbonyl;
and with the further proviso that in said compound of Formula (III), Z¹ is not a 3-(4-trifluoromethyl-pyridinyl) group or a 3-(4-trifluoromethyl-pyridinyl N-oxide) group;
and with the further proviso that in said compound of Formula (III), Z¹ is not a substituted or unsubstituted group selected from the group consisting of

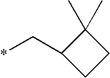

and a —(CH₂)ₜ-adamantanyl group, wherein t is 0-4;
and with the further proviso that in said compound of Formula (III), Z² is not a

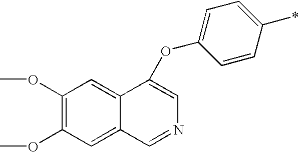

group, which is unsubstituted, or which is substituted with one or more substituents independently selected from the group consisting of halogens and alkyl groups;
and with the further proviso that in said compound of Formula (III), Z² is not a substituted or unsubstituted group selected from the group consisting of:

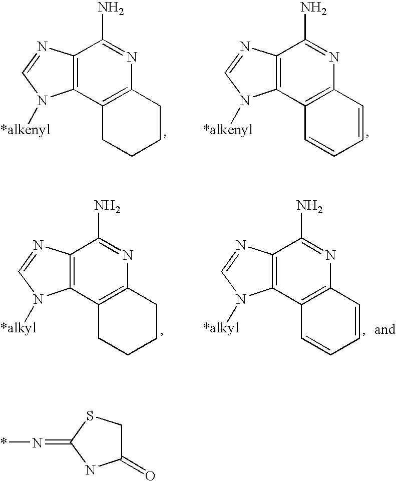

and with the further proviso that said compound of Formula (III), is not a compound set forth in Appendix A, Appendix B, or Appendix C.

In another embodiment, a compound of the present invention is a replicase complex defect inducer identified by the method of screening a test compound for replicase complex defect inducer activity comprising: providing a test compound; contacting the test compound with an isolated HCV polyprotein or fragment thereof; and identifying the test compound as an inducer of an HCV replicase complex defect when the level of p14 protein is increased, wherein said compound is a compound of Formula (III)

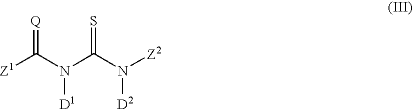

wherein
Q is oxygen or sulfur;
D¹ and D² are independently selected from the group consisting of hydrogen and methyl;
with the proviso that said compound of Formula (III) is not a compound of the formula (III-a):

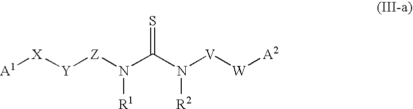

wherein

A₁ and A₂ are independently optionally substituted C₁-C₁₂ alkyl, optionally substituted mono- or di-(C₁-C₈ alkyl)amino, optionally substituted C₂-C₁₂ alkenyl, optionally substituted C₃-C₈ cycloalkyl, an optionally substituted partially unsaturated or aromatic carbocyclic group, or an optionally substituted saturated, partially unsaturated, or aromatic heterocyclic group, wherein at least one of A₁ and A₂ is an optionally substituted aromatic carbocyclic group or an optionally substituted aromatic heterocyclic group;

X and W are independently O, S, NR, or absent, wherein R is hydrogen, optionally substituted (C₁-C₆)alkyl, or optionally substituted aryl(C₀-C₄)alkyl;

V is (C₁-C₆)alkyl, (C₂-C₆)alkenyl, (C₃-C₇)cycloalkyl, or absent, wherein when V is absent, W is absent; and

Y is (C₁-C₆) alkyl, (C₁-C₆)alkyl substituted with (C₃-C₇)cycloalkyl, (C₂-C₆) alkenyl, (C₃-C₇)cycloalkyl, or absent; and

Z is carbonyl, thiocarbonyl, imino, or C₁-C₆ alkylimino; and

R₁ and R₂ are independently hydrogen or methyl;

and with the further proviso that said compound of Formula (II) is not a compound of the formula (III-b):

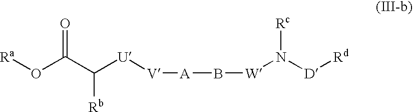

wherein
R^{a} is hydrogen, a substituted or unsubstituted alkyl or cycloalkyl group, a substituted or unsubstituted aryl group or a saturated or unsaturated, optionally substituted heterocyclic group;
R^{b} is hydrogen, a substituted or unsubstituted alkyl or cycloalkyl group, a substituted or unsubstituted aryl group, a saturated or unsaturated, optionally substituted heterocyclic group, an optionally substituted alkenyl group, an optionally substituted alkinyl group, —NR^{2′}SO₂R^{2″}, —NR^{2′}COOR^{2′}, —NR^{2′}COR^{2′}, —NR^{2′}CON(R^{2′})₂, or —N R^{2′}CSN(R^{2′})₂;
R^{2′} is hydrogen, a substituted or unsubstituted alkyl or cycloalkyl group, a substituted or unsubstituted aryl group or a saturated or unsaturated, optionally substituted heterocyclic group;
R^{2″} is a substituted or unsubstituted alkyl, alkenyl or cycloalkyl group, a substituted or unsubstituted aryl group, or a saturated or unsaturated, optionally substituted heterocyclic group;
U′ is a direct bond or a substituted or unsubstituted alkylene group;
V′ is a substituted or unsubstituted alkylene group, —NR^{2′}CO—, or —NR^{2′}SO₂—;
A and B are each independently an optionally substituted 1,3- or 1,4-bridging phenylene group or an optionally substituted 2,4- or 2,5-bridging thienylene group;
W′ is a direct bond or an optionally substituted alkylene group;
D′ is a direct bond or

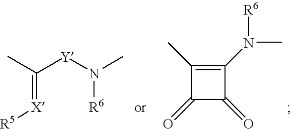

R^{c} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{d}, Y′, R⁵, or R⁶, if present, with formation of an optionally substituted heterocyclic ring system which includes the nitrogen atom to which R^{c} is bonded, and may be saturated or unsaturated and may contain further heteroatoms;
R^{d} is hydrogen, an optionally substituted alkyl or cycloalkyl group, an optionally substituted aryl group, a saturated or unsaturated, optionally substituted heterocyclic group, an alkylamine group, an alkylamide group or is connected to one of R^{c}, Y, R⁵ or R⁶, if present, with formation of an optionally substituted heterocyclic ring system which includes the nitrogen atom to which R^{d} is bonded and may be saturated or unsaturated and may contain further heteroatoms;

### X′ is CHNO₂, CHCN, O, N or S;

Y′ is a direct bond or an optionally substituted alkylene or alkine group;
R⁵ is absent, or is hydrogen, a substituted or unsubstituted alkyl or cycloalkyl group, —NO₂, —CN, —COR^{5′}, —COOR^{5′}, or is connected to one of R^{c}, Y′, R^{d}, or R⁶, if present, with formation of an optionally substituted carbocyclic or heterocyclic ring system which includes X′ and can be saturated or unsaturated and may optionally contain one or more additional heteroatoms;
R^{5′} is hydrogen, a substituted or unsubstituted alkyl or cycloalkyl group, a substituted or unsubstituted aryl group or a saturated or unsaturated, optionally substituted heterocyclic group which may optionally contain one or more additional heteroatoms;
R⁶ is a arylcarbonyl group, or a heteroarylcarbonyl group;
and wherein if A is a phenylene group and V′ is —NR^{2′}CO— or NR^{2′}SO₂—, D′ is not a direct bond and X′ is not N;
and with the further proviso that said compound of Formula (III) is not a compound of the formula (III-c)

X^{1″}—Y^{1″}-Z^{1″}-W^{1″} (III-c)

wherein
X^{1″} is optionally substituted aryl, optionally substituted (C₁-C₈)alkyl-aryl, optionally substituted heteroaryl, or optionally substituted heteroarylalkyl;
Y^{1″} is —NR^{A}CSNR^{A}CO—, wherein R^{A} is independently hydrogen or lower alkyl;
Z^{1″} is optionally substituted phenylene, optionally substituted monocyclic heteroarylene, optionally substituted monocyclic non-aromatic heterocycle-diyl, or optionally substituted monocyclic cycloalkane-diyl;
W^{1″} is a group represented by the formula:

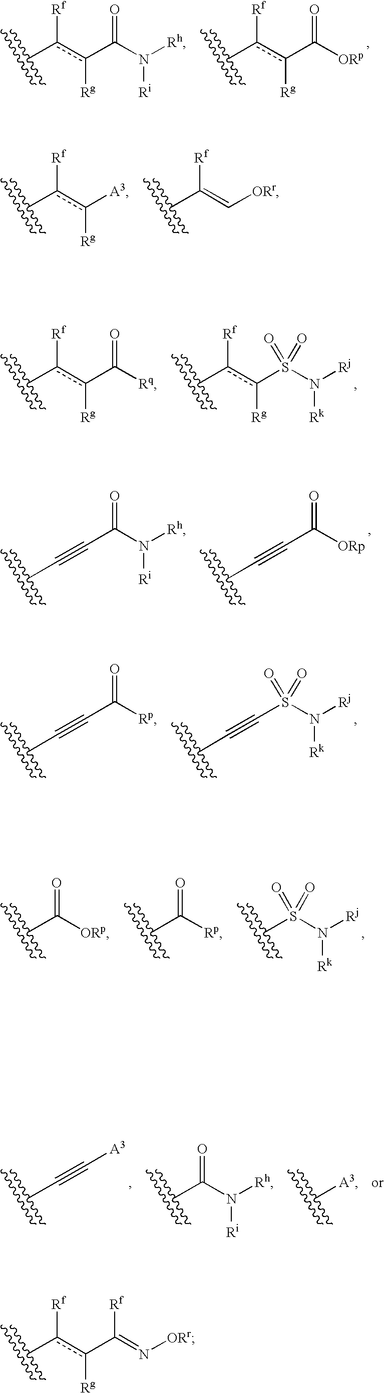

wherein
R^{f}, R^{g}, R^{h}, Rⁱ, R^{j}, and R^{k} are each independently hydrogen, halogen, optionally substituted lower alkyl, optionally substituted lower alkyloxy, optionally substituted lower alkylthio, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, optionally substituted non-aromatic heterocyclic group, or optionally substituted amino;
R^{p}, R^{q}, and R^{r} are each independently hydrogen, optionally substituted lower alkyl, optionally substituted lower alkenyl, optionally substituted lower alkynyl, optionally substituted aryl, optionally substituted heteroaryl, optionally substituted cycloalkyl, optionally substituted (C₁-C₈)alkyl-aryl, optionally substituted heteroarylalkyl, or optionally substituted non-aromatic heterocyclic group;
A³ is an optionally substituted aryl or an optionally substituted heteroaryl group;
and with the further proviso that in said compound of Formula (III), Z¹ is not a 3-pyridyl group or a substituted 3-pyridyl group when Z² is —OH, —NH₂, —(C₁-C₆)alkylamino, di(C₁-C₆)alkylamino, an alkyl group, a substituted alkyl group, —(C₃-C₈) cycloalkyl group, a substituted —(C₃-C₈) cycloalkyl group, —(C₃-C₈) cycloalkyl-(C₁-C₆)alkyl group, —O-alkyl group, a substituted —O-alkyl group, —O—(C₃-C₈) cycloalkyl group, a substituted —O—(C₃-C₈) cycloalkyl group, a phenyl group, a fused-phenyl group, a phenyl group substituted with one or more independently selected halogen, —OCH₃, nitro, or dimethyl amino groups, a substituted fused-phenyl group substituted with one or more independently selected halogen, —OCH₃, nitro, or dimethyl amino groups, a —(C₁-C₈) alkyl-phenyl group, —O—(C₀-C₈) alkyl-phenyl group, —(C₃-C₆) alkylene group, —O—(C₃-C₆) alkylene group, —(C₃-C₆) alkynyl group, —O—(C₃-C₆) alkynyl group, —CO(C₁-C₆) alkyl group, —NHCO(C₁-C₆) alkyl group, —NHSO₂(C₁-C₆) alkyl group, —SO₂(C₁-C₆) alkyl group, or —SO₂(C₀-C₆)alkyl-phenyl group;
and with the further proviso that in said compound of Formula (III), Z¹ is not a furyl group or a substituted furyl group when D² and Z² are independently selected from the group consisting of hydrogen and methyl;
and with the further proviso that in said compound comprising structure (III), when D² is —CH₃ and Z² is an optionally substituted phenyl, Z¹ is not selected from the group consisting of —R³³, OR³⁴, and —NR³⁵R³⁶
wherein
R³³ is hydrogen, optionally substituted (C₃-C₈)cycloalkyl, optionally substituted phenyl, optionally substituted heteroaryl or phenyl-(C₁-C₄) alkyl which is optionally substituted on the phenyl ring, (C₁-C₆)alkyl, (C₂-C₆)alkenyl, (C₂-C₆)alkynyl, the three latter radicals independently being optionally substituted with one or more radicals selected from the group consisting of halogen, (C₁-C₄)alkoxy, (C₁-C₄)alkylthio and NR³⁷R³⁸;
R³⁴ is (C₁-C₆)alkyl, (C₂-C₆)alkenyl, (C₂-C₆)alkynyl, the three latter radicals independently optionally substituted with one or more radicals selected from the group consisting of halogen, (C₁-C₄)alkoxy, (C₁-C₄)alkylthio and NR³⁷R³⁸, or R³⁴ is (C₃-C₆)cycloalkyl optionally substituted with one or more groups selected from the group consisting of halogen, (C₁-C₄)alkyl and (C₁-C₄)alkoxy, and (C₃-C₆)-cycloalkyl-(C₁-C₃)alkyl;
R³⁵ and R³⁶ are hydrogen, (C₁-C₆)alkyl, (C₂-C₆)alkenyl, (C₂-C₆)alkynyl, the 3 latter groups independently optionally substituted by one or more halogen radicals;
R³⁷ is independently selected from the group consisting of hydrogen, (C₁-C₄)alkyl, ((C₁-C₄)alkyl)carbonyl, ((C₁-C₄)alkoxy)carbonyl and CHO;
R³⁸ is independently selected from the group consisting of H and (C₁-C₄)alkyl;
and with the further proviso that in said compound of Formula (III), Z¹ and Z² are not both selected from the group consisting of hydrogen and alkyl;
and with the further proviso that in said compound of Formula (III), Z² is not hydrogen when D¹ and D² are both hydrogen;
and with the further proviso that in said compound of Formula (III), Z¹ is not (C₁-C₆) alkyl, (C₁-C₆) alkoxy, pyridyl, or aryl when D¹ and D² are both hydrogen and when Z² is selected from the group consisting of hydrogen, a (C₁-C₆) alkyl group optionally substituted with 1 to 3 substituents independently selected from the group consisting of hydroxyl, (C₁-C₆) alkoxyl, carboxyl, (C₁-C₆) alkoxycarbonyl, lower alkylthio, fluorine, chlorine, bromine, iodine, amino, mono- or di-substituted amino, phthalimido and nitrogen-containing heterocyclic groups, a (C₃-C₈) cycloalkyl group, a (C₂-C₆) alkenyl group, an arylalkyl group optionally substituted with from 1 to 5 substituents independently selected from the group consisting of (C₁-C₆) alkyl, hydroxyl, (C₁-C₆) alkoxy, halogen, nitro, and amino groups, an aryl group optionally substituted with from 1 to 5 independently selected substituents selected from the group consisting of (C₁-C₆) alkyl, hydroxyl, (C₁-C₆) alkoxy, halogen, nitro, and amino groups, a substituted or unsubstituted heterocyclic group, a (C₁-C₆) alkanoyl group, a benzoyl or a naphthoyl group either of which is optionally substituted with from 1 to 5 independently selected substituents selected from the group consisting of (C₁-C₆) alkyl, hydroxyl, (C₁-C₆) alkoxy, halogen, nitro, and amino groups, a pyridylcarbonyl group, a (C₁-C₆) alkoxycarbonyl group or an amino group optionally substituted with (C₁-C₆) alkyl, arylalkyl having 7 to 15 carbon atoms, substituted or unsubstituted aryl, (C₁-C₆) alkanoyl, optionally substituted aroyl, and (C₁-C₆) alkylidene groups;
and with the further proviso that in said compound of Formula (III), Z¹ is not —O—(CH₂)ₜ—CH₃, wherein t is 0-4 and Z² is halophenyl;
and with the further proviso that in said compound of Formula (III), Z¹ is not a substituted pyridyl, pyrimidinyl, pyrazinyl, pyridazinyl group substituted with —N(R²¹)-(optionally substituted phenyl), wherein said R²¹ is hydrogen, aryl, formyl, (C₁-C₆)alkylcarbonyl, (C₁-C₆)alkyl, (C₁-C₆)alkyloxycarbonyl, (C₁-C₆)alkyl substituted with formyl, (C₁-C₆)alkylcarbonyl, (C₁-C₆)alkyloxycarbonyl, (C₁-C₆)alkylcarbonyloxy, or (C₁-C₆)alkyloxy(C₁-C₆)alkylcarbonyl optionally substituted with (C₁-C₆)alkyloxycarbonyl;
and with the further proviso that in said compound of Formula (III), Z¹ is not a 3-(4-trifluoromethyl-pyridinyl) group or a 3-(4-trifluoromethyl-pyridinyl N-oxide) group;
and with the further proviso that in said compound of Formula (III), Z¹ is not a substituted or unsubstituted group selected from the group consisting of

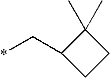

and a —(CH₂)ₜ-adamantanyl group, wherein t is 0-4;
and with the further proviso that in said compound of Formula (III), Z² is not a

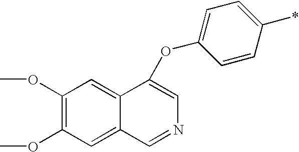

group, which is unsubstituted, or which is substituted with one or more substituents independently selected from the group consisting of halogens and alkyl groups;
and with the further proviso that in said compound of Formula (III), Z² is not a substituted or unsubstituted group selected from the group consisting of:

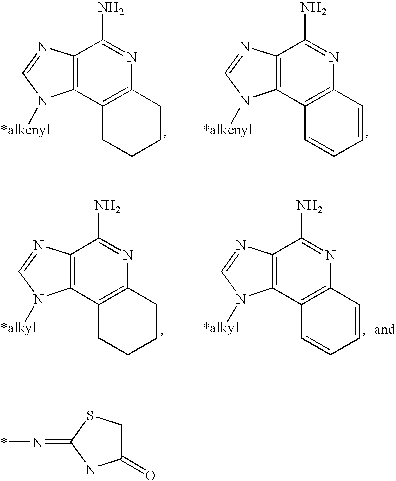

and with the further proviso that said compound of Formula (III), is not a compound set forth in Appendix A, Appendix B, or Appendix C.

In another embodiment of the present invention, a compound identified by a method of the present invention is a compound of Formula (III), wherein D′ is methyl. In another embodiment, a compound identified by a method of the present invention is a compound of Formula (III), wherein D² is methyl. In another embodiment, a compound identified by a method of the present invention is a compound of Formula (III), wherein D¹ and D² are both methyl.

In a further embodiment, a compound identified by a method of the present invention is a compound of Formula (III), wherein Z¹ is optionally substituted phenyl. In another embodiment, a compound identified by a method of the present invention is a compound of Formula (III), wherein Z¹ is not optionally substituted phenyl. In a further embodiment, a compound identified by a method of the present invention is a compound of Formula (III), wherein Z² is optionally substituted phenyl. In another embodiment, a compound identified by a method of the present invention is a compound of Formula (III), wherein Z² is not optionally substituted phenyl.

In another embodiment, a compound identified by a method of the present invention is a compound of Formula (III), wherein Z¹ is optionally substituted pyridyl. In another embodiment, a compound identified by a method of the present invention is a compound of Formula (III), wherein Z¹ is not optionally substituted pyridyl. In a further embodiment, a compound identified by a method of the present invention is a compound of Formula (III), wherein Z² is optionally substituted pyridyl. In another embodiment, a compound identified by a method of the present invention is a compound of Formula (III), wherein Z² is not optionally substituted pyridyl.

In another embodiment, a compound identified by a method of the present invention is a compound of Formula (III), wherein Z¹ and Z² are not optionally substituted phenyl or optionally substituted pyridyl.

In another embodiment, a compound identified by a method of the present invention is a compound of Formula (III), wherein D¹ is methyl and Z¹ is phenyl or pyridyl. In another embodiment, a compound identified by a method of the present invention is a compound of Formula (III), wherein D² is methyl and Z¹ is phenyl or pyridyl.

In another embodiment, a compound identified by a method of the present invention is a compound of Formula (III), wherein D¹ is methyl and Z² is optionally substituted phenyl or optionally substituted pyridyl. In another embodiment, a compound identified by a method of the present invention is a compound of Formula (III), wherein D² is methyl and Z² is optionally substituted phenyl or optionally substituted pyridyl.

In another embodiment, a compound identified by a method of the present invention is a compound of Formula (III), wherein D¹ is methyl and Z¹ and Z² are not optionally substituted phenyl or optionally substituted pyridyl. In another embodiment, a compound identified by a method of the present invention is a compound of Formula (III), wherein D² is methyl and Z¹ and Z² are not optionally substituted phenyl or optionally substituted pyridyl.

As used herein, aryl includes single and multiple carbocyclic aromatic rings that may be fused or bound to one or more saturated, unsaturated or aromatic carbocyclic or heterocyclic rings. An example of a fused aryl ring system is a fused-phenyl group such as benzofuran.

As used herein, heteroaryl includes single and multiple aromatic rings having one or more endocyclic heteroatoms, wherein the single or multiple aromatic rings may be fused or bound to one or more saturated, unsaturated or aromatic carbocyclic or heterocyclic rings.

As used herein, carbocyclic may be spiro, fused, or bridged with one or more carbocyclic or heterocyclic groups, wherein the one or more carbocyclic or heterocyclic groups may be saturated, unsaturated, or aromatic.

A replicase complex defect inducer may be identified by the methods described herein (see e.g., Methods of the Present Invention and Examples as described infra).

### B. Methods of the Present Invention

Methods of Making and Screening:

The present invention includes and provides a method of identifying a mutant that is resistant to a replicase complex defect inducer comprising: growing a cell that expresses an HCV virion, an HCV replicon, an isolated HCV replicase complex or an isolated HCV polyprotein or fragment thereof; contacting the cell with a test compound; and identifying a mutant that is resistant to a test compound and sensitive to an NS5B polymerase inhibitor and an NS3 protease inhibitor.

The present invention includes and provides a method of identifying a mutant that is resistant to a replicase complex defect inducer comprising: growing a cell that expresses an HCV virion, an HCV replicon, an isolated HCV replicase complex or an isolated HCV polyprotein or fragment thereof; contacting the cell with a selection agent and a test compound; and identifying a mutant that is resistant to a test compound and sensitive to an NS5B polymerase inhibitor and an NS3 protease inhibitor. The present invention includes and provides a method of identifying a mutant that is resistant to a replicase complex defect inducer comprising: growing a cell that expresses an HCV virion, an HCV replicon, an isolated HCV replicase complex or an isolated HCV polyprotein or fragment thereof; contacting the cell with hygromycin and a test compound; and identifying a mutant that is resistant to a test compound and sensitive to an NS5B polymerase inhibitor and an NS3 protease inhibitor.

The present invention includes and provides a method of identifying a mutant that is resistant to a replicase complex defect inducer comprising: growing a cell that expresses an HCV virion, an HCV replicon, an isolated HCV replicase complex or an isolated HCV polyprotein or fragment thereof; contacting the cell with G418 and a test compound; and identifying a mutant that is resistant to a test compound and sensitive to an NS5B polymerase inhibitor and an NS3 protease inhibitor. The present invention also provides compositions, such as for example virions, replicase complexes, and polyproteins, that are identified by this method.

Identifying a mutant as resistant to a replicase complex defect inducer may be achieved in any manner by which resistance of a mutant to an RCDI can be determined. In a preferred embodiment, a mutant is identified as resistant to an RCDI by its growth in the presence of an RCDI, for example, as described in Example 1.

In an embodiment of the present invention, resistance of a cell to a replicase complex defect inducer refers to the ability of a cell to grow in the presence of an RCDI. Susceptibility or sensitivity to an RCDI refers to the inability or reduced ability of a cell to grow in the presence of an RCDI. Determination of resistance or susceptibility of a cell to a compound may be accomplished, for example in a preferred embodiment, by determining EC₅₀, EC₉₀, or both of an RCDI.

In an embodiment, a resistant clone may show an average change in EC₅₀ for a defect inducer of about 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 10.5-fold, 11-fold, 11.5-fold, 12-fold, 12.5-fold, 13-fold, 15-fold, 20-fold, 30-fold, 40-fold, 50-fold, 100-fold, or more than about 125-fold as compared with a wild type clone. An average change in EC₅₀ levels between wild type and mutant clones of the present invention also include ranges in which the lower limit is selected from the following changes: 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 10.5-fold, 11-fold, 11.5-fold, 12-fold, 12.5-fold, 13-fold, 15-fold, 20-fold; and the upper limit is selected from the following changes: 15-fold, 20-fold, 30-fold, 40-fold, 50-fold, 100-fold, 125-fold, 150-fold, 175-fold, 200-fold, 225-fold, and 250-fold. As used herein, any range set forth is inclusive of the end points of the range unless otherwise stated. Any untreated clone, such as a cell expressing an untreated HCV replicon, may be considered a wild type clone. Moreover, a wild type clone can contain any of the exemplary preferred replicons provided herein before treatment. In a preferred embodiment, EC₅₀ may be measured by a dot blot assay.

In an embodiment, resistance may be evidenced for example by greater growth of a mutant clone in the presence of an RCDI relative to the growth of a wild type clone in the presence of the same RCDI. In another embodiment, resistance may be evidenced by an increase in viral mRNA in a mutant in the presence of an RCDI as compared with a wild type in the presence of the same RCDI.

In another embodiment of the present invention, resistance of an HCV virion, an HCV replicon, an HCV replicase complex or an HCV polypeptide or fragment thereof to an RCDI refers to the ability of a cell to grow in the presence of an RCDI. Determination of resistance of an HCV virion, HCV replicon, HCV replicase complex or HCV polypeptide or fragment thereof to an RCDI may be accomplished, for example, by comparing mRNA levels in an HCV virion, HCV replicon, HCV replicase complex or HCV polypeptide or fragment thereof before and after treatment with the RCDI. In another embodiment, determination of resistance of an HCV virion, HCV replicon, HCV replicase complex or HCV polypeptide or fragment thereof to an RCDI may be accomplished, for example, by comparing mRNA levels from an HCV virion, HCV replicon, HCV replicase complex or HCV polypeptide or fragment thereof after treatment with an RCDI with wild type mRNA levels of the HCV virion, HCV replicon, HCV replicase complex or HCV polypeptide or fragment thereof.

Resistance to an RCDI may result, for example, from any mutation in an HCV virion, an HCV replicon, an HCV replicase complex, or an HCV polyprotein or fragment thereof. Any amino acid may be substituted for any other amino acid to cause resistance to a replicase complex defect inducer. A mutation includes any change in one or more amino acids of an HCV protein or fragment thereof in an HCV replicon, an HCV replicase complex, or an HCV polyprotein. A mutation includes a change in 1 or more, 2 or more, 3 or more, 5 or more, 10 or more, 25 or more, or more than about 50 amino acids. In a preferred embodiment, the mutation interferes with functional replicase complex assembly. Resistance to an RCDI can also result, for example, from a change in the amino acid family, as differentiated by the side chain. The common amino acids are grouped according to whether their side chains are basic, acidic, uncharged polar, or nonpolar. Based on comparisons of more than one amino acid sequence of an NS3-NS4A protein, conserved amino acid residues can be identified in a different NS3-NS4A genotype or strain, which when mutagenized at the site corresponding to the known mutant, alter the resistance to an RCDI in the different NS3-NS4A protein. Engineering of a mutant NS3, resistant to an RCDI, can result from aligning in more than one protein where the amino acid is conserved at a residue corresponding to the C16 or A39 of SEQ ID NO: 1.

In an embodiment of the present invention, a mutation that causes resistance to an RCDI is due to a mutation in the NS3 protein or fragment thereof. In another embodiment, a mutation that causes resistance to an RCDI is due to a mutation in the helicase protein or fragment thereof. In another embodiment, a mutation that causes resistance to an RCDI is due to a mutation in the NS4A protein or fragment thereof. In an embodiment, a mutation that causes resistance to an RCDI is not due to a mutation in the NS3 protein or fragment thereof, helicase protein or fragment thereof, or NS4A protein or fragment thereof. In another embodiment, resistance to an RCDI is due to more than one mutation in the same or different proteins or fragments thereof.

In an embodiment, a mutation is a mutation at or within about 40 or less, 30 or less, 25 or less, 15 or less, 10 or less, or 5 or less Angstroms from the C16 (cysteine at the 16^{th} position) of NS3. Exemplary amino acids that are found within about 15 Angstroms or less from NS3 are listed in Table 1 below. In an embodiment, any of the amino acids shown in Table 1 may be substituted with any other amino acid including alanine, cysteine, aspartic acid, glutamic acid, phenylalanine, glycine, histidine, isoleucine, lysine, leucine, methionine, asparagine, proline, glutamine, arginine, serine, threonine, valine, tryptophan, or tyrosine in order to confer resistance to an RCDI. In addition, it is contemplated that in an embodiment, an amino acid in Table 1 or any other amino acid may be substituted with any nonnatural amino acid that can exist in HCV.

TABLE 1
NS3 Amino Acids Within 15 Å of Cys 16

| | |
|---|---|
| Amino Acid | Position |

NS3 protease

| | |
|---|---|
| Gln | 8 |
| Gln | 9 |
| Thr | 10 |
| Arg | 11 |
| Gly | 12 |
| Leu | 13 |
| Leu | 14 |
| Gly | 15 |
| Cys | 16 |
| Ile | 17 |
| Ile | 18 |
| Thr | 19 |
| Ser | 20 |
| Leu | 21 |
| Thr | 22 |
| Gly | 23 |
| Arg | 24 |
| Asp | 25 |
| Lys | 26 |
| Asn | 27 |
| Gln | 28 |
| Val | 29 |
| Val | 33 |
| Gln | 34 |
| Val | 35 |
| Val | 36 |
| Ser | 37 |
| Thr | 38 |
| Ala | 39 |
| Thr | 40 |
| Gln | 41 |
| Ser | 42 |
| Phe | 43 |
| Leu | 44 |
| Ala | 45 |
| Cys | 52 |
| Thr | 54 |
| Gly | 58 |
| Ala | 59 |
| Gly | 60 |
| Lys | 62 |
| Thr | 63 |
| Leu | 64 |
| Ala | 65 |
| Gly | 66 |
| Pro | 67 |
| Lys | 68 |
| Glv | 69 |
| Pro | 70 |
| Ile | 71 |
| Trp | 85 |
| Pro | 88 |
| Arg | 109 |
| Gly | 137 |
| Ser | 139 |

Helicase

| | |
|---|---|
| Pro | 478 |
| Gly | 479 |

NS4A Cofactor

| | |
|---|---|
| Gly | 705 |
| Ser | 706 |
| Val | 707 |
| Val | 708 |
| Ile | 709 |
| Val | 710 |
| Gly | 711 |
| Arg | 712 |

In a preferred embodiment of the present invention, resistance to an RCDI is due to a mutation at alanine in the 39^{th} position in an HCV NS3 protein or fragment thereof. In another preferred embodiment, resistance to an RCDI results from a mutation at cysteine in the 16^{th} position in an HCV NS3 protein or fragment thereof. In a highly preferred embodiment, resistance to an RCDI results from an A39V mutation in an HCV NS3 protein or fragment thereof. An A39V mutation reflects an alanine to valine mutation at the 39^{th} position. In another highly preferred embodiment, resistance to an RCDI results from a C16S mutation in an HCV NS3 protein or fragment thereof. A portion of the wild type sequence including C16 and A39 from NS3 protein is provided as SEQ ID NO: 1. A portion of the nucleic acid sequence encoding NS3 protein from exemplary mutants with an A39V mutation is provided in SEQ ID NOs: 2-5. A portion of the nucleic acid sequence encoding NS3 protein from exemplary mutants with a C16S mutation is provided in SEQ ID NOs: 6 and 7. The present invention includes and provides nucleic acid molecules identical over their entire length to each coding sequence as set forth in the Sequence Listing. The present invention further includes and provides fragments of each coding sequence as set forth in the Sequence Listing, wherein the fragment is capable of increasing resistance of a clone to a replicase complex defect inducer.

The present invention includes and provides a method of identifying a mutant that is resistant to a replicase complex defect inducer. In the context of the present invention, a mutant includes any cell comprising an HCV virion, an HCV replicon, an isolated HCV replicase complex, or an isolated HCV polyprotein or fragment thereof that comprises any mutation from its native state, including by way of non-limiting example the mutations described supra. A mutation may be produced by any means known to the artisan. A particularly preferred method for introducing a mutation is provided in Example 1. A replicase complex defect inducer includes any inhibitor of functional replicase complex assembly as described in more detail, supra, in the section entitled “Replicase complex defect inducers (RCDIs)”.

The present invention contemplates growing a cell that expresses an HCV replicon. A replicon is a genetic element, including by way of non-limiting example, a plasmid, cosmid, bacmid, phage or virus or any portion of the foregoing that is capable of replication largely under its own control. A replicon may be either RNA or DNA and may be single- or double-stranded. A replicon may contain a positive nucleic acid strand, a negative nucleic acid strand or both. In a preferred embodiment, an HCV replicon comprises the NS5B nonstructural protein of an HCV genome. In another preferred embodiment, an HCV replicon comprises the NS3-NS4A nonstructural proteins of an HCV genome. In another preferred embodiment, an HCV replicon comprises the NS3-NS5B nonstructural proteins of an HCV genome. In a further preferred embodiment, one or more HCV nonstructural proteins is operably linked to sequences necessary for efficient replication.

It is contemplated that any HCV replicon may be used in the methods of the present invention. In a preferred embodiment, a hepatitis C virus RNA replicon can be used in the methods of the present invention. Without limitation, Con-1 replicons, replicons derived from HCV H77 strain (subtype 1a), HCV N strain (subtype 1b), and JFH-1 (subtype 2a) may be used in the methods of the present invention. In one embodiment, any of the genotypes 1, 2, 3, 4, 5, and 6 can be used in the methods of the present invention, Several exemplary preferred replicons are provided. GenBank Accession Numbers AJ242654 (SEQ ID NO: 8), AJ242653 (SEQ ID NO: 9), AJ242652 (SEQ ID NO: 10), AJ242651 (SEQ ID NO: 11) also provide exemplary replicons of the present invention. Further exemplary replicons of the present invention may be found at viral accession numbers AF009606 (SEQ ID NO: 12), AF011751 (SEQ ID NO: 13), and AF139594 (SEQ ID NO: 14) and replicon accession number AB114136 (SEQ ID NO: 15). Other replicons including Con-1 replicons generated from the plasmids of SEQ ID NO: 16 through SEQ ID NO: 27 may be used in the methods of the present invention. Any other replicon available to the art worker may be used. In a preferred embodiment, a Con-1 replicon is used.

An HCV replicon may be obtained in any manner. For example, RNA molecules encoding an HCV replicon may be produced by in vitro transcription and transfected into cells such as by electroporation. In another embodiment, the HCV replicon may be DNA that is transfected. An HCV replicon may be transfected into any cells known to the skilled artisan. In a preferred embodiment, an HCV replicon is transfected into Huh-7 cells using electroporation. In another preferred embodiment, an HCV replicon is obtained from an accession database such as GenBank or ATCC.

The present invention also contemplates growing a cell that expresses an isolated HCV replicase complex. Any isolated HCV replicase complex may be used in the methods of the present invention. Replicase complexes may be isolated in any manner known to the skilled artisan. Replicase complexes may be isolated for example as described in Example 9 or in Lohmann, V. et al., Replication of subgenomic hepatitis C virus RNAs in a hepatoma cell line, Science 285:110-113 (1999); Blight, K. J., et al., Efficient replication of hepatitis C virus genotype 1a RNAs in cell culture, J. Virol. 77(5) 3181-90 (2003); Wolk, B. et al., Subcellular localization stability, and trans-cleavage competence of the hepatitis C virus NS3-NS4A complex expressed in tetracycline-regulated cell lines, J. Virol. 74(5): 2293-2304 (2000).

Exemplary replicase complexes include those that comprise an NS5B protein or fragment thereof, an NS3-NS5B polyprotein or fragment thereof, or an NS3-NS4A polyprotein or fragment thereof. In the context of the present invention, a replicase complex that is isolated includes one that is removed or separated from its natural environment. Any techniques for removing a replicase complex from the location where it is naturally found may be used for isolation, including for example extraction, fractionation, centrifugation, precipitation, etc.

An isolated replicase complex may optionally be purified from other components. For example, an isolated replicase complex may be about 70% free, about 75% free, about 80% free, about 85% free, about 90% free, about 95% free, about 98% free, about 99% free, about 99.5% free, or more than about 99.5% free of other components by weight.

The present invention also contemplates growing a cell that expresses an isolated HCV polyprotein or fragment thereof. Any isolated HCV polyprotein or fragment thereof may be used in the methods of the present invention. HCV polyproteins may be isolated for example as described in Example 9 or in Lohmann, V. et al., Replication of subgenomic hepatitis C virus RNAs in a hepatoma cell line, Science 285:110-113 (1999); Blight, K. J., et al., Efficient replication of hepatitis C virus genotype 1a RNAs in cell culture, J. Virol. 77(5) 3181-90 (2003); Wolk, B. et al., Subcellular localization stability, and trans-cleavage competence of the hepatitis C virus NS3-NS4A complex expressed in tetracycline-regulated cell lines, J. Virol. 74(5): 2293-2304 (2000).

Exemplary polyproteins or fragments thereof of the present invention include those that comprise an NS5B protein or fragment thereof, an NS3-NS5B polyprotein or fragment thereof, or an NS3-NS4A polyprotein or fragment thereof. In the context of the present invention, a polyprotein or fragment thereof that is isolated includes one that is removed or separated from its natural environment. An isolated polyprotein or fragment thereof may optionally be purified from other components. For example, an isolated polyprotein or fragment thereof may be about 70% free, about 75% free, about 80% free, about 85% free, about 90% free, about 95% free, about 98% free, about 99% free, about 99.5% free, or more than about 99.5% free of other components by weight.

In the methods of the present invention, any cell that is capable of expressing an HCV virion, an HCV replicon, an HCV replicase complex, or an HCV polyprotein or fragment thereof can be used to express the same. In a preferred embodiment, an Huh-7 cell is used to express an HCV virion, an HCV replicon, an HCV replicase complex, or an HCV polyprotein or fragment thereof. Growth of a cell may be conducted in any manner known to the skilled art worker for maintaining life of the cell. In a preferred embodiment, maintenance medium contains (DMEM (Dulbecco's modified Eagle media) supplemented with 10% FBS, L-glutamine, non-essential amino acids, penicillin (100 units/ml), streptomycin (100 micrograms/ml), and 500 micrograms/ml of Geneticin (G418)).

In a preferred embodiment, high levels of viral RNA replication are achieved by using a suitable density of cells in order to express the HCV replicon. In an embodiment, a suitable density of cells refers to a density of cells that is not overly confluent. In an embodiment, a suitable density of cells is found in a liquid culture where cells are in log phase growth. A suitable density of cells will be known to the artisan and may depend upon the culture medium that is used. In an embodiment, a richer culture medium supports a higher suitable density of cells.

In an embodiment, a suitable density of cells expressing the HCV replicon in liquid culture is about 1-2×10⁶ cells/mL. In another embodiment, a suitable density of cells in liquid culture is about 1-2×10⁷ cells/mL. In another embodiment, a suitable density of cells in liquid culture is about 1-2×10⁸ cells/mL. In another embodiment, a suitable density of cells in liquid culture is about 1-2×10⁹ cells/mL. In another embodiment, a suitable density of cells in liquid culture is about 1-2×10¹⁰ cells/mL. In another embodiment, a suitable density of cells in liquid culture is about 1-2×10¹¹ cells/mL. In another embodiment, a suitable density of cells in liquid culture is about 1-2×10¹² cells/mL. Cells may be examined under a microscope to ensure that they cells are growing well and have reached a suitable density. In another embodiment, cell density may be measured by spectrophotometry. In a preferred embodiment, cells may be passed twice a week at 1: 4-6 dilution to maintain suitable cell density.

G418 (also known as Genticin®) provides selection conditions resulting in the production of adaptive mutations that are necessary for cell growth but that do not affect the function of the HCV replicon (see e.g., Lohmann et al., (2001) J. Virol. 75(3), 1487-1499). G418 can be added in any concentration to produce adaptive mutations. In an embodiment, G418 can be added in a concentration of about 10 micrograms/mL to about 10000 micrograms/mL. In another embodiment, G418 can be added in a concentration of about 100 micrograms/mL to about 1000 micrograms/mL, about 250 micrograms/mL to about 750 micrograms/mL. In a preferred embodiment, Huh-7 cells expressing an HCV replicon, an isolated HCV replicase complex, or an isolated HCV polyprotein or fragment thereof may be grown in log phase in the presence of 500 micrograms/ml of G418 and a test compound.

In an embodiment of the present invention, a cell expressing an HCV virion, an HCV replicon, an isolated HCV replicase complex, or an isolated HCV polyprotein or fragment thereof is contacted with a test compound. In another embodiment of the present invention, a cell expressing an HCV virion, an HCV replicon, an isolated HCV replicase complex, or an isolated HCV polyprotein or fragment thereof is contacted with a test compound and any other compound, referred to herein as a selection agent, that can produce an adaptive mutation without affecting the replicating function of the HCV virion, HCV replicon, replicase complex or polyprotein. In an embodiment of the present invention, a selection agent is hygromycin. In another embodiment, a selection agent is G418.

In a preferred embodiment of the present invention, a cell expressing an HCV virion, an HCV replicon, an isolated HCV replicase complex, or an isolated HCV polyprotein or fragment thereof is contacted with G418 and a test compound. G418 or a test compound or both may be contacted with a cell expressing a hepatitis C virus RNA replicon in any manner that permits the test compound and the cell comprising the replicon to interact. A test compound may be contacted with a cell expressing a hepatitis C virus RNA replicon by mixing the test compound and the cell together in any container such as for example a flask, a replicate plate, a tube, or a vial.

A test compound includes any compound that may be tested for activity as a replicase complex defect inducer. A test compound includes, for example, a chemical, nucleic acid, polypeptide, amino acid, or any other compound that is to be tested for activity as an RCDI. Examples of test compounds include, but are not limited to, drug candidates, such as derived from arrays of small molecules generated through general combinatorial chemistry, as well as any other substances thought to have potential biological activity. Non-limiting exemplary test compounds are provided herein, for example in Appendix A.

Test compounds may also include metabolites of other test compounds. A metabolite is a compound that has been metabolized in vivo. Compounds that have been metabolized include compounds resulting for example from the oxidation, reduction, hydrolysis, amidation, esterification and the like of a test compound. Metabolite structures can be determined in any fashion, including for example by conventional techniques such as MS, NMR, or IR analysis.

In a preferred embodiment, a test compound has not previously been screened for induction of an HCV replicase complex defect. In another embodiment, a test compound has not previously been identified as an inducer of an HCV replicase complex defect. In a further preferred embodiment, a test compound has neither been previously screened nor identified as an inducer of an HCV replicase complex defect.

In a preferred embodiment, a test compound is an acylthiourea or a metabolite thereof.

A compound may be selected as a test compound randomly or on the basis of any information available to the skilled art worker. In an embodiment, a test compound is selected on the basis of experience of an artisan, structure of the compound, structural activity relationship data, EC₅₀, assay data, IC₅₀ assay data, animal or clinical studies, or any other basis, or combination of such bases.

Test compounds for use in the methods of the present invention or identified by the methods of the present invention as useful pharmacological agents can be pharmacological agents already known in the art or variations thereof or can be compounds previously unknown to have any pharmacological activity. Test compounds can be naturally occurring or designed or modified in the laboratory.

Test compounds can comprise a single diastereomer, more than one diastereomer, a single enantiomer, or more than one enantiomer. In a preferred embodiment, a test compound comprises a single diastereomer, whose replicase complex defect inducing activity is greater than the inhibitory activity on any other diastereomers of that test compound. In another preferred embodiment, a test compound comprises an enantiomer, whose replicase complex defect inducing activity is greater than the inhibitory activity of any other enantiomers of that test compound. In another preferred embodiment, a test compound comprises a single diastereomer. In another preferred embodiment, a test compound comprises a single enantiomer.

Test compounds can be isolated, as from microorganisms, animals or plants, for example, and can be produced recombinantly, or synthesized by chemical methods known in the art. If desired, test compounds of the present invention can be obtained using any of the numerous combinatorial library methods known in the art, including but not limited to, biological libraries, spatially addressable parallel solid phase or solution phase libraries, synthetic library methods requiring deconvolution, the “one-bead one-compound” library method, and synthetic library methods using affinity chromatography selection. The biological library approach is limited to polypeptide libraries. The other four approaches are applicable to polypeptide, non-peptide oligomer, or small molecule libraries of compounds and are preferred approaches in the present invention. See Lam, Anticancer Drug Des. 12: 145-167 (1997).

In producing a library of test compounds for use in the methods of the present invention, many synthesis methods are well known in the art and may be used (see, for example, DeWitt et al., Proc. Nat. Acad. Sci. USA 90: 6909-6913 (1993); Erb et al. Proc. Natl. Acad. Sci. U.S.A. 91: 11422 (1994); Zuckermann et al., J. Med. Chem. 37: 2678 (1994); Cho et al., Science 261: 1303 (1993); Carell et al., Angew. Chem. Int. Ed. Engl. 33: 2059 (1994); Carell et al., Angew. Chem. Int. Ed. Engl. 33: 2061 (1994); Gallop et al., J. Med. Chem. 37: 1233 (1994)). Libraries of compounds can be presented in solution (see, e.g., Houghten, BioTechniques 13: 412-421 (1992)), or on beads (Lam, Nature 354: 82-84 (1991)), chips (Fodor, Nature 364: 555-556 (1993)), bacteria or spores (Ladner et al., U.S. Pat. No. 5,223,409), plasmids (Cull et al., Proc. Natl. Acad. Sci. USA 89, 1865-1869 1992), or phage (Scott & Smith, Science 249: 386-390 (1990); Devlin, Science 249: 404-406 (1990)); Cwirla et al., Proc. Natl. Acad. Sci. USA, 97: 6378-6382 (1990); Felici, J. Mol. Biol. 222: 301-310 (1991); and Ladner et al., U.S. Pat. No. 5,223,409).

The present invention also includes and provides a method of identifying a mutation that results in growth of cells in the presence of an HCV replicase complex defect inducer comprising: generating a population of mutants comprising a mutation in a nonstructural protein of HCV; identifying a mutant that is resistant to a test compound and sensitive to an NS5B polymerase inhibitor and an NS3 protease inhibitor; and determining the nucleotide sequence of the mutation.

A population of mutants may contain any number of mutants. For example, about 10 or more, about 10² or more, about 10³ or more, about 10⁴ or more, about 10⁵ or more, about 10⁶ or more, about 10⁷ or more, about 10⁸ or more, about 10⁹ or more, about 10¹⁰ or more, about 10¹¹ or more, or about 10¹² or more mutants may be generated. The population of mutants may contain multiple different mutations, multiple copies of the same mutation, or a combination thereof. In a preferred embodiment, a population of mutants contains multiple different mutations.

A population of mutants that comprise one or more mutation in a nonstructural protein of HCV may be generated by any techniques known to the artisan. In a preferred embodiment, adaptive mutations necessary for cell growth are produced but the mutations do not affect the function of the HCV replicon. In a preferred embodiment, a mutation is generated by the use of G418 and a test compound. Example 1 provides an illustrative example of generating a mutation in a nonstructural protein of HCV.

Identifying a mutant that is resistant to a test compound and sensitive to an NS5B polymerase inhibitor and an NS3 protease inhibitor may be achieved by any techniques available to the skilled artisan. Particularly preferred embodiments for evaluating resistance and sensitivity to a variety of inhibitors are provided in Example 1 and include, for example, determination of EC₅₀ and EC₉₀.

In order to determine the nucleotide or polypeptide sequence of a mutation in a mutant resistant to a test compound, conventional sequencing techniques may be employed. For example, two methods available for DNA sequencing include the chain termination method of Sanger et al., Proc. Natl. Acad. Sci. (U.S.A.) 74: 5463-5467 (1977), the entirety of which is herein incorporated by reference and the chemical degradation method of Maxam and Gilbert, Proc. Nat. Acad. Sci. (U.S.A.) 74: 560-564 (1977), the entirety of which is herein incorporated by reference.

Advances in technology such as the replacement of radioisotopes with fluorescence-based sequencing have reduced the effort required to sequence DNA (Craxton, Methods, 2: 20-26 (1991), the entirety of which is herein incorporated by reference; Ju et al., Proc. Natl. Acad. Sci. (U.S.A.) 92: 4347-4351 (1995), the entirety of which is herein incorporated by reference; Tabor and Richardson, Proc. Natl. Acad. Sci. (U.S.A.) 92: 6339-6343 (1995), the entirety of which is herein incorporated by reference). In addition, automation has facilitated DNA sequencing. Automated sequencing machines are available from, for example, Pharmacia Biotech, Inc., Piscataway, N.J. (Pharmacia ALF), LI-COR, Inc., Lincoln, Nebr. (LI-COR 4,000) and Millipore, Bedford, Mass. (Millipore BaseStation).

The present invention also includes and provides a method of determining resistance to a test compound comprising: introducing into a cell an HCV virion, an HCV replicon, an isolated HCV replicase complex, or an isolated HCV polyprotein or fragment thereof comprising a mutation; contacting a test compound and the cell; and measuring the resistance of the cell or the HCV virion, replicon, replicase complex or polyprotein to the test compound.

Any HCV virion, replicon, replicase complex or polyprotein may comprise a mutation as a result of any technique by which a mutation is introduced such as for example by selection with G418 as discussed supra and at Example 1. An HCV virion, replicon, isolated HCV replicase complex, or HCV polyprotein or fragment thereof comprising a mutation may be introduced into a cell by any technique available to the skilled artisan.

Technology for introduction of nucleic acid molecules, including an HCV replicon or a nucleic acid molecule encoding an HCV polypeptide or fragment thereof into cells is well known to those of skill in the art. Several general methods for delivering a nucleic acid into cells have been described such as without limitation chemical methods; physical methods including microinjection (Capecchi, Cell 22:479-488 (1980)), electroporation (Wong and Neumann, Biochem. Biophys. Res. Commun. 107:584-587 (1982); Fromm et al., Proc. Natl. Acad. Sci. (U.S.A.) 82:5824-5828 (1985); U.S. Pat. No. 5,384,253); the gene gun (Johnston and Tang, Methods Cell Biol. 43:353-365 (1994)); and viral vectors (Clapp, Clin. Perinatol. 20:155-168 (1993); Lu et al., J. Exp. Med. 178:2089-2096 (1993); Eglitis and Anderson, Biotechniques 6:608-614 (1988)).

Acceleration methods that may be used include, for example, microprojectile bombardment and the like. One example of a method for delivering transforming nucleic acid molecules is microprojectile bombardment. Non-biological particles (microprojectiles) may be coated with nucleic acids and delivered into cells by a propelling force. Exemplary particles include those comprised of tungsten, gold, platinum and the like. A particle delivery system suitable for use with the invention is the helium acceleration PDS-1000/He gun is available from Bio-Rad Laboratories (Bio-Rad, Hercules, Calif.) (Sanford et al., Technique 3:3-16 (1991)).

It is contemplated that one may wish to adjust various aspects of the bombardment parameters in small-scale studies to fully optimize the conditions. One may particularly wish to adjust physical parameters such as gap distance, flight distance, tissue distance and helium pressure. One may also minimize the trauma reduction factors by modifying conditions which influence the physiological state of the recipient cells and which may therefore influence transformation and integration efficiencies. For example, the osmotic state, tissue hydration and the subculture stage or cell cycle of the recipient cells may be adjusted for optimum transformation. The execution of other routine adjustments will be known to those of skill in the art in light of the present disclosure.

Nucleic acid molecules of the present invention may also be introduced by electroporation. In a preferred embodiment of the present invention, an RNA molecule is introduced into cell by electroporation. Parameters for electroporation such as gap width, nucleic acid concentration, cell concentration and pulse strength are well known to those skilled in the art. In addition, exemplary electroporation conditions are described without limitation in Example 3. In a preferred embodiment, an RNA molecule is introduced into an Huh-7 cell by electroporation.

The present invention includes contacting a test compound with a cell. As described, supra, a test compound may be contacted with a cell expressing a hepatitis C virus RNA replicon in any manner that permits the test compound and the cell comprising the replicon to interact.

The present invention also includes and provides a method of screening a test compound for replicase complex defect inducer activity comprising: providing a test compound; contacting the test compound with a cell that expresses an HCV virion, an HCV replicon, an isolated HCV replicase complex, or an isolated HCV polyprotein or fragment thereof; and identifying the test compound as an inducer of an HCV replicase complex defect.

Any resources available to the skilled artisan may be used in order to provide a test compound. For example, a test compound may be provided by purchasing, synthesizing, extracting, purifying, etc. the test compound.

A test compound may be identified as an inducer of an HCV replicase complex defect by any available means. In an embodiment, production of a product may be ascertained by observing an increased level of a protein as compared to the level of that protein produced in the absence of a replicase complex defect inducer. In another embodiment, production of a protein may be determined by observing a decreased level of a protein as compared to the level of that protein produced in the absence of a replicase complex defect inducer.

In a preferred embodiment, an inducer of an HCV replicase complex defect may be identified by increased production of a product, such as for example a p14 product or NS4A* product described in Examples 6 and 7 or a variant or fragment thereof. A product may be identified where production of any protein is increased by about 5%, 10%, 25%, 50%, 100%, 200%, 500%, or by more than about 1000% as compared with the protein level in untreated HCV.

A defect inducer may also be identified by decreased production of a protein that is produced in HCV replication where the HCV virion, replicon, replicase complex or polyprotein or fragment thereof is untreated by a selective agent. For example, an inducer of an HCV replicase complex defect may be identified by decreased production of a normal product, such as for example an NS3 or NS4A protein product as described in Example 6. A product may be identified where production of a normal protein is decreased by about 5%, 10%, 25%, 50%, 100%, 200%, 500%, or by more than about 1000% as compared with the protein level in untreated HCV replication.

In another embodiment, a replicase complex defect inducer may be identified where both resistance to the test compound and susceptibility to a protease or polymerase inhibitor are observed. For example, in a preferred embodiment, a test compound may be identified as an RCDI where resistance to the test compound and susceptibility to an NS5B inhibitor or an NS3-NS4A inhibitor are demonstrated as, without limitation, in Example 1.

In another embodiment, the present invention provides a method of inhibiting hepatitis C virus replication comprising providing a replicase complex defect inducer compound and contacting said replicase complex defect inducer compound with a hepatitis C virus, replicon, replicase complex, polyprotein or a fragment thereof, wherein replication of said hepatitis C virus is inhibited, and wherein said replicase complex defect inducer does not inhibit the active site of a hepatitis C virus protease or enzymatic activity of a hepatitis C virus polymerase.

Inhibition of HCV replication may be measured by a decrease in nucleotide or protein production and includes a reduction in HCV replication of at least about 10%, at least about 25%, at least about 35%, at least about 45%, at least about 50%, at least about 55%, at least about 60%, at least about 65%, at least about 70%, at least about 75%, at least about 80%, at least about 85%, at least about 90%, at least about 95%, at least about 98%, or at least about 99% as compared with HCV replication in the absence of RCDI.

Methods of Treatment:

In an embodiment of the present invention, a method of treatment of a subject in need thereof is provided, the method comprising administering an effective amount of a replicase complex defect inducer compound, where the subject is treated. In an embodiment, the present invention provides a method of treatment of a subject that has liver disease, comprising administering an effective amount of a replicase complex defect inducer compound, where the subject that has liver disease is treated. In the context of the present invention, a subject is any living organism that may benefit from treatment for a disease or condition. For example, a subject includes without limitation mammals such as dogs, cats, cows, horses, rabbits, monkeys, and humans. In a preferred embodiment, a subject is a human. Subjects that may benefit from treatment include those that have been diagnosed with a disease or condition, those that are suspected of having a disease or condition, or those that may be susceptible to a disease or condition. Benefits of treatment may include prevention of a disease or condition or amelioration of a disease or condition, including elimination of a disease or condition.

A subject that has liver disease includes any subject that has any manifestation of liver dysfunction. In addition, a subject that has liver disease further includes any subject that has a history of any disease that is associated with liver dysfunction. A disease that is associated with liver dysfunction is a disease for which it is known or suspected that the liver may be affected. Liver dysfunction may be determined by clinical evaluation, laboratory testing, pathology report, or any other means available to the skilled artisan. In the context of the present invention, a subject that has liver disease may have, without limitation, acute hepatitis C viral infection, chronic hepatitis, liver cancer, cirrhosis of the liver, end-stage liver disease, or any combination thereof. A subject that has liver disease includes a liver transplant patient. A subject that has liver disease includes any subject that has antibodies to hepatitis C virus. In a preferred embodiment, a subject that has liver disease has antibodies to hepatitis C virus.

The present invention contemplates that an RCDI may be administered to a subject in order to achieve a therapeutic effect. For administration, an RCDI of the present invention may be formulated into any appropriate pharmaceutical composition. A composition can be administered to a subject alone, or in combination with other pharmaceutical agents.

In addition to the active ingredients, the pharmaceutical compositions of the present invention can contain suitable pharmaceutically-acceptable excipients, including without limitation, carriers, solvents, stabilizers, adjuvants, and diluents. Suitable excipients may include large, slowly metabolized macromolecules such as proteins, polysaccharides, polylactic acids, polyglycolic acids, polymeric amino acids, amino acid copolymers, and inactive virus particles. Other exemplary excipients include antioxidants such as ascorbic acid; chelating agents such as EDTA; carbohydrates such as dextrin, hydroxyalkylcellulose, hydroxyalkylmethylcellulose, and stearic acid; liquids such as oils, water, saline, glycerol and ethanol; wetting or emulsifying agents; pH buffering substances; and the like. Liposomes are also included within the definition of pharmaceutically acceptable excipients.

An RCDI of the present invention can be administered in any sterile, biocompatible pharmaceutical carrier, including saline, buffered saline, dextrose, and water, for example. Pharmaceutical compositions of the invention may further comprise any additional ingredients such as flavoring or preservatives. Pharmaceutical compositions of the invention can be administered by any route including, but not limited to, oral, intravenous, intramuscular, intra-arterial, intramedullary, intrathecal, intraventricular, transdermal, subcutaneous, intraperitoneal, intranasal, parenteral, topical, sublingual, or rectal administration.

The pharmaceutical compositions should generally be formulated to achieve a physiologically compatible pH, and may range from a pH of about 3 to a pH of about 11, or about pH 3 to about pH 7, depending on the formulation and route of administration. In alternative embodiments it may be preferred that the pH is adjusted to a range from about pH 5 to about pH 8.

Formulations of the present invention, e.g., for parenteral or oral administration, are most typically solids, liquid solutions, emulsions or suspensions, while inhaleable formulations for pulmonary administration are generally liquids or powders, with powder formulations being generally preferred. Pharmaceutical compositions of the invention may also be formulated as a lyophilized solid that is reconstituted with a physiologically compatible solvent prior to administration. Additional pharmaceutical compositions of the invention may be formulated as syrups, creams, ointments, tablets, and the like.

Pharmaceutical formulations suitable for parenteral administration can be formulated in aqueous solutions, preferably in physiologically compatible buffers such as Hanks' solution, Ringer's solution, or physiologically buffered saline. Aqueous injection suspensions can contain substances that increase the viscosity of the suspension, such as sodium carboxymethyl cellulose, sorbitol, or dextran. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers also can be used for delivery. Optionally, the suspension also can contain suitable stabilizers or agents which increase the solubility of the compounds to allow for the preparation of highly concentrated solutions.

The pharmaceutical compositions of the present invention can be manufactured in a manner that is known in the art, e.g., by means of conventional mixing, dissolving, granulating, dragee-making, levigating, emulsifying, encapsulating, entrapping, or lyophilizing processes. Further details on techniques for formulation and administration can be found in the latest edition of REMINGTON'S PHARMACEUTICAL SCIENCES (Maack Publishing Co., Easton, Pa.).

The present invention includes administration of an effective amount of a replicase complex defect inducer. An effective amount is an amount of active ingredient (ie., RCDI) that decreases HCV replication. A decrease in HCV replication may be a decrease of any amount. A decrease in HCV replication may be measured by a decrease in nucleotide or protein production and includes a reduction in HCV replication of at least about 10%, at least about 25%, at least about 35%, at least about 45%, at least about 50%, at least about 55%, at least about 60%, at least about 65%, at least about 70%, at least about 75%, at least about 80%, at least about 85%, at least about 90%, at least about 95%, at least about 98%, or at least about 99% as compared with HCV replication in the absence of RCDI.

In determining effective amount, the clinician may assess one, some or many criteria relating to the compound that may affect the activity of the compound as a therapeutic agent. Factors such as, for example, efficacy, safety, efficiency, retention, localization, tissue selectivity, degradation, or intracellular persistence may be considered in determining an effective amount. For any replicase complex defect inducer, the effective amount can be estimated initially either in cell culture assays or in animal models, usually mice, rabbits, dogs, or pigs. The animal model also can be used to determine the appropriate concentration range and route of administration. Such information can then be used to determine useful doses and routes for administration in humans.

Therapeutic efficacy and toxicity, e.g., ED₅₀ (the dose therapeutically effective in 50% of the population) and LD₅₀ (the dose lethal to 50% of the population), can be determined by standard pharmaceutical procedures in cell cultures or experimental animals. The dose ratio of toxic to therapeutic effects is the therapeutic index and can be expressed as the ratio, LD₅₀/ED₅₀.

The dosage contained in RCDI pharmaceutical compositions is preferably within a range of circulating concentrations that include the ED₅₀ with little or no toxicity. The dosage varies within this range depending upon the dosage form employed, sensitivity of the patient, and the route of administration.

The exact dosage will be determined by the practitioner, in light of factors related to the subject that will be treated. Dosage and administration are adjusted to provide sufficient levels of the active ingredient or to maintain the desired effect. Factors which can be taken into account include for example the severity of the disease state, general health of the subject, age, weight, and gender of the subject, diet, time and frequency of administration, drug combination(s), reaction sensitivities, and tolerance/response to therapy. Pharmaceutical compositions of the invention may be administered daily, one or more times per day, two or more times per day, three or more times per day, four or more times per day, or continuously. Long-acting pharmaceutical compositions can be administered every 3 to 4 days, every week, once every two weeks, once a month, or less frequently depending on the half-life and clearance rate of the particular formulation.

Normal dosage amounts can vary from between 0.01 pmoles/kg body weight/minute to 10000 pmoles/kg body weight/minute, depending upon the route of administration. Guidance as to particular dosages and methods of delivery is provided in the literature and generally available to practitioners in the art. Delivery of a pharmaceutical composition may targeted to particular cells, organs, or locations.

In any of the embodiments described above, any of the pharmaceutical compositions of the invention can be administered in combination with other appropriate therapeutic agents. Selection of the appropriate agents for use in combination therapy can be made by one of ordinary skill in the art, according to conventional pharmaceutical principles. The combination of therapeutic agents can act synergistically to effect the treatment or prevention of the various disorders described above. Using this approach, one may be able to achieve therapeutic efficacy with lower dosages of one or more of the agents, thus reducing the potential for adverse side effects.

Any replicase complex defect inducer may be used in the pharmaceutical compositions of the present invention. Preferred RCDIs include those provided herein, such as for example Test Compounds A, B, C, D, and E and others as described in the Examples. Further preferred RCDIs include those identified by the methods of the present invention.

### C. Nucleic Acid Molecules and Polypeptides of the Present Invention

Nucleic Acid Molecules:

The present invention includes and provides nucleic acid molecules related to replicase complex defect induction. As used herein, nucleic acids include both single- and double-stranded DNA and RNA molecules. Nucleic acids may be linear or circular.

Nucleic acid molecules that are related to replicase complex defect induction include any nucleic acid molecule that is associated with replication, including those molecules that are part of or encode part of an HCV replicon, replicase complex, or polyprotein. Those molecules that are involved in inhibition of replication, and those molecules that are produced or inhibited as a result of replication or inhibition thereof are also nucleic acid molecules related to induction of a replicase complex defect. Nucleic acid molecules related to replicase complex defect induction include replicase complex defect inducers.

Nucleic acid molecules of the present invention also include those nucleic acids that encode a polypeptide of the present invention. For example, a nucleic acid molecule encoding the p14 protein, NS4A* protein or a variant or fragment of either is a nucleic acid molecule of the present invention and is related to replicase complex defect induction for purposes of the present invention. In an embodiment, a nucleic acid molecule encoding an NS3 protein or a fragment thereof with an A39V mutation is a nucleic acid molecule that is related to induction of a replicase complex defect. In another embodiment, a nucleic acid molecule encoding NS3 protein or a fragment thereof with a C16S mutation is a nucleic acid molecule that is related to induction of a replicase complex defect. In an embodiment, the present invention includes, for example, an isolated nucleic acid molecule that encodes an NS3 protein or a fragment thereof with an A39V mutation, or a C16S mutation, or both.

One subset of the nucleic acid molecules of the invention is fragment nucleic acids molecules, such as for example a fragment of a nucleic acid molecule that encodes an HCV polyprotein. In a preferred embodiment, a fragment comprises a nucleic acid molecule that encodes a polypeptide or fragment thereof comprising A39V mutation. Fragment nucleic acid molecules may be used, for example, as probes or primers as described infra. Fragments may consist of significant portions of, or indeed most of, the nucleic acid molecules of the invention. Alternatively, the fragments may comprise smaller oligonucleotides, for example oligonucleotides having from about 15 to about 400 nucleotide residues and more preferably, about 15 to about 30 nucleotide residues, or about 50 to about 100 nucleotide residues, or about 100 to about 200 nucleotide residues, or about 200 to about 400 nucleotide residues, or about 275 to about 350 nucleotide residues.

In the context of the present invention, nucleic acids include probes and primers, such as for example those capable of producing a cDNA from an HCV coding region. Such probes or primers can themselves be or can be derived from the nucleic acid molecules of the present invention. The term probe as used herein refers to a polynucleotide, whether occurring naturally or produced synthetically, which is capable of specifically hybridizing to a nucleic acid molecule. A probe may be either single-stranded or double-stranded, but is preferably single-stranded. A probe may be, for example, a single-stranded RNA transcribed in vitro from a DNA template. The exact length of a probe will depend upon many factors, including temperature, source of the probe, and use. An appropriate probe or primer length may be determined empirically by the skilled artisan.

Nucleic acids of the present invention also include nucleic acids that are complementary to other nucleic acids described herein. A nucleic acid molecule is said to be the complete complement of another nucleic acid molecule if the two molecules exhibit complete complementarity. As used herein, molecules are said to exhibit complete complementarity when every nucleotide of one of the molecules is complementary to a nucleotide of the other. Two molecules are said to be minimally complementary if they can hybridize to one another with sufficient stability to permit them to remain annealed to one another under at least conventional low-stringency conditions. Similarly, the molecules are said to be complementary if they can hybridize to one another with sufficient stability to permit them to remain annealed to one another under conventional high-stringency conditions. With respect to nucleic acid molecules, as used herein, two nucleic acid molecules are said to be capable of specifically hybridizing to one another if the two molecules are capable of forming an anti-parallel, double-stranded nucleic acid structure.

Conventional stringency conditions are described by Sambrook et al., Molecular Cloning, A Laboratory Manual, 2^{nd} Ed., Cold Spring Harbor Press, Cold Spring Harbor, N.Y. (1989) and by Haymes et al., Nucleic Acid Hybridization, A Practical Approach, IRL Press, Washington, D.C. (1985). Departures from complete complementarity are therefore permissible, as long as such departures do not completely preclude the capacity of the molecules to form a double-stranded structure. Thus, in order for a nucleic acid molecule to serve as a primer or probe it need only be sufficiently complementary in sequence to be able to form a stable double-stranded structure under the particular solvent and salt concentrations employed.

Appropriate stringency conditions, which promote DNA hybridization, for example, 6.0× sodium chloride/sodium citrate (SSC) at about 45° C., followed by a wash of 2.0×SSC at 20-25° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 65° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed.

A nucleic acid of the present invention may be isolated. Isolated nucleic acid molecules include those molecules that are separated from an intact cellular environment. An isolated nucleic acid molecule may be, for example, a viral replicon RNA that is separated from the cell nucleus, chromosomal DNA, and other cellular materials that are not membrane-associated.

An isolated nucleic acid molecule, such as a viral replicon RNA molecule, can be substantially free of other cellular material or culture medium when produced by recombinant techniques, or substantially free of chemical precursors or other chemicals when chemically synthesized. A nucleic acid molecule that is substantially free of other components may be about 70% free, about 75% free, about 80% free, about 85% free, about 90% free, about 95% free, about 98% free, about 99% free, about 99.5% free, or more than about 99.5% free of other components by weight. In an embodiment, an isolated viral replicon RNA may be isolated as a portion of a membrane fraction of a cell expressing a viral replicon RNA. Such a membrane fraction may also comprise isolated replicase complexes.

In an embodiment, an isolated nucleic acid is free of sequences which flank the nucleic acid in the genomic DNA or RNA of the organism from which the nucleic acid is derived (e.g., sequences located at the 5′ or 3′, or both 5′ and 3′ ends of the nucleic acid). For example, an isolated nucleic acid molecule may contain less than about 5 kb, less than about 4 kb, less than about 3 kb, less than about 2 kb, less than about 1 kb, less than about 0.5 kb or less than about 0.1 kb of either 5′ or 3′ nucleotide sequences or both 5′ and 3′ nucleotide sequences which flank the nucleic acid molecule in genomic DNA of the cell from which the nucleic acid is derived. An isolated nucleic acid may be, for example, a DNA vector encoding a viral replicon RNA, which isolated nucleic acid has been purified by standard DNA purification methods.

Also contemplated are natural allelic variants and mutants of nucleic acids of the present invention. Natural allelic variants and mutants refer to nucleic acid sequences that are closely related to a particular sequence but that may possess, either naturally or by design, some changes in sequence. By closely related, it is meant that greater than or equal to about 70%, greater than or equal to about 75%, greater than or equal to about 80%, greater than or equal to about 85%, greater than or equal to about 90%, or greater than or equal to about 95% of the nucleotides of two nucleotide sequences match over the length of the two sequences.

Match over the length of two sequences may be measured by the Needleman-Wunch algorithm (1970). The CLUSTAL X program may be used in making sequence alignments. CLUSTAL X is a multiple sequence alignment package available that performs progressive multiple sequence alignments based on the method of Feng and Doolittle, J. Mol. Evol. 25: 351-360 (1987), the entirety of which is herein incorporated by reference. Each pair of sequences is aligned and the distance between each pair is calculated; from this distance matrix, a guide tree is calculated, and all of the sequences are progressively aligned based on this tree. A feature of the program is its sensitivity to the effect of gaps on the alignment; gap penalties are varied to encourage the insertion of gaps in probable loop regions instead of in the middle of structured regions. Users can specify gap penalties, choose between a number of scoring matrices, or supply their own scoring matrix for both the pairwise alignments and the multiple alignments.

CLUSTAL X is available for a number of different platforms including: SUN Solaris, IRIX5.3 on Silicon Graphics, Digital UNIX on DECStations, Microsoft Windows (32 bit) for PC's, Linux ELF for x86 PC's and Macintosh PowerMac. In a preferred embodiment, CLUSTAL X version 1.7 is used with the following default parameters:

### DNA Sequence Parameters:

### Fast Pairwise Alignment Parameters:

K-tuple (word) size: 2
Window size: 4
Scoring method: percentage
Number of top diagonals: 4
Gap penalty: 5

### Multiple Alignment Parameters:

Gap opening penalty: 10.0
Gap extension penalty: 5.0
Weight transitions: Yes

Changes or differences in nucleotide sequence between closely related nucleic acids may arise during the course of normal replication or duplication of a nucleic acid sequence. Other changes may be specifically designed and introduced into the sequence intentionally, such as for example, in order to change an amino acid codon or sequence in the nucleic acid. Such changes may be made in vitro using a variety of mutagenesis techniques or may be produced in a host organism placed under conditions that induce or select for the changes. Such sequence variants may be referred to as mutants of the original sequence.

In another embodiment of the present invention, one or more of the nucleic acid molecules of the present invention differ in nucleic acid sequence from those encoding a enzyme or fragment thereof due to the fact that one or more codons encoding an amino acid has been substituted for by a codon that produces the same amino acid originally encoded. Techniques of conservative substitution that may be employed may be those apparent to the artisan as well as those described, for example, herein infra.

Different variants including, for example, natural allelic variants of the HCV genome exist in nature. These variants may be alleles characterized by differences in the nucleotide sequences of the gene coding for a protein, or may involve different RNA processing or post-translational modifications. The skilled artisan can produce variants having single or multiple amino acid substitutions, deletions, additions or replacements. These variants may include, inter alia, a) variants in which one or more amino acids residues are substituted with conservative or non-conservative amino acids, b) variants in which one or more amino acids are added, or c) variants in which one or more amino acids include a substituent group.

In another embodiment of the invention, one or more of the nucleic acid molecules or fragments thereof of the present invention share between about 100% and 70% sequence identity with one or more other polynucleotides of the present invention. In a further embodiment of the invention, one or more of the polynucleotide molecules of the invention shares between about 100% and 90% sequence identity with one or more other polynucleotides of the present invention. In an embodiment of the invention, one or more of the polynucleotide molecules of the invention shares between about 100% and 95% sequence identity with one or more other polynucleotides of the present invention. In another embodiment of the invention, one or more of the polynucleotides of the invention shares between about 100% and 99% sequence identity with one or more other polynucleotides of the present invention.

For example, in an embodiment, one or more of the nucleic acid molecules or fragments thereof of the present invention share between about 100% and 70% sequence identity with a nucleic acid molecule that encodes an HCV NS3 protein or fragment thereof comprising an A39V mutation or a C16S mutation. In a further embodiment of the invention, one or more of the polynucleotide molecules of the invention shares between about 100% and 90% sequence identity with a nucleic acid molecule that encodes an HCV NS3 protein or fragment thereof comprising an A39V mutation or a C16S mutation. In an embodiment of the invention, one or more of the polynucleotide molecules of the invention shares between about 100% and 95% sequence identity with a nucleic acid molecule that encodes an HCV NS3 protein or fragment thereof comprising an A39V mutation or a C16S mutation. In another embodiment of the invention, one or more of the polynucleotides of the invention shares between about 100% and 99% sequence identity with a nucleic acid molecule that encodes an HCV NS3 protein or fragment thereof comprising an A39V mutation or a C16S mutation.

The compounds of the present invention also include nucleic acid molecules that are fused to one another. In an embodiment, a nucleic acid molecule of the present invention may be operatively linked to other nucleic acid sequences, such as for example promoters, enhancers, transcription terminators, start codons, intron splicing signals, leader sequences and stop codons.

The present invention also includes nucleic acid molecules that are introduced into host cells. In an embodiment, an RNA molecule of the present invention encoding an HCV replicon may be transformed into host cells. In another embodiment, a DNA molecule comprising the HCV replicon may be transformed into host cells. By the present invention, any technique that accomplishes effective transformation may be used. For example, techniques such as electroporation, transfection, injection, or bombardment are contemplated. In a preferred embodiment, a nucleic acid molecule is transformed into Huh-7 cells.

Polypeptides:

In another embodiment, the present invention includes polypeptides. As used herein, a polypeptide is any molecule that has three or more amino acid molecules joined by peptide bonds. A polypeptide may contain any additional chemical groups and may be folded into any conformation.

The present invention includes and provides polypeptides related to replicase complex defect induction. Polypeptide molecules that are related to induction of a replicase complex defect include any polypeptide molecule that is associated with replication, including those molecules that are part of a replicase complex, those molecules that are involved in inhibition of replication, and those molecules that are produced or inhibited as a result of replication or inhibition thereof. In an embodiment, polypeptides that are related to induction of a replicase complex defect include replicase complex defect inducers. In an embodiment, the p14 protein, the NS4A* protein and variants and fragments thereof as described herein are polypeptides related to replicase complex defect induction for purposes of the present invention.

In an embodiment of the present invention, a polypeptide not detected in normal HCV replication is detected in the presence of a replicase complex defect inducer. In a further embodiment of the present invention, a polypeptide of the present invention is produced in a greater quantity than previously observed when in the presence of a replicase complex defect inducer. In a preferred embodiment, a p14 protein, an NS4A* protein or variant or fragment of either is produced in the presence of a replicase complex defect inducer.

Another embodiment of the present invention includes fragments of polypeptides. Fragments of a polypeptide may consist of significant polypeptide sequences, or indeed most of the polypeptide sequences of, the enzymes of the present invention. Alternatively, the fragments may comprise smaller polypeptides, for example, having from about 3 to about 150 amino acids and more preferably, about 5 to about 15 amino acids, or about 20 to about 40 amino acids, or about 40 to about 70 amino acids, or about 70 to about 150 amino acids, or about 120 to 150 amino acids, or about 90 to about 120 amino acids.

A polypeptide of the present invention may be isolated. Isolated polypeptides include those molecules that are separated from an intact cellular environment. An isolated polypeptide may be, for example, a viral replicon protein that is separated from the cell nucleus, chromosomal DNA, and other cellular materials that are not membrane-associated.

An isolated polypeptide can be substantially free of other cellular material or culture medium when produced by recombinant techniques, or substantially free of chemical precursors or other chemicals when chemically synthesized. A polypeptide molecule that is substantially free of other components may be about 70% free, about 75% free, about 80% free, about 85% free, about 90% free, about 95% free, about 98% free, about 99% free, about 99.5% free, or more than about 99.5% free of other components by weight.

Homologs are also included in the present invention. As used herein, a homolog or a fragment thereof is a counterpart molecule or fragment thereof in another species. A homolog can also be generated by molecular evolution or DNA shuffling techniques, so that the molecule retains at least one functional or structural characteristic of the original polypeptide (see e.g., U.S. Pat. No. 5,811,238).

In another embodiment of the invention, one or more of the polypeptide molecules of the present invention share between about 100% and 70% sequence identity with one or more other polypeptides of the present invention. In a further embodiment of the invention, one or more of the polypeptide molecules of the invention shares between about 100% and 90% sequence identity with one or more other polypeptides of the present invention. In an embodiment of the invention, one or more of the polypeptide molecules of the invention shares between about 100% and 95% sequence identity with one or more other polypeptides of the present invention. In another embodiment of the invention, one or more of the polypeptides of the invention shares between about 100% and 99% sequence identity with one or more other polypeptides of the present invention.

For example, in an embodiment, one or more of the polypeptide molecules or fragments thereof of the present invention share between about 100% and 70% sequence identity with an HCV NS3 protein or fragment thereof comprising an A39V mutation or a C16S mutation. In a further embodiment of the invention, one or more of the polypeptide molecules of the invention shares between about 100% and 90% sequence identity with an HCV NS3 protein or fragment thereof comprising an A39V mutation or a C16S mutation. In an embodiment of the invention, one or more of the polypeptide molecules of the invention shares between about 100% and 95% sequence identity with an HCV NS3 protein or fragment thereof comprising an A39V mutation or a C16S mutation. In another embodiment of the invention, one or more of the polypeptides of the invention shares between about 100% and 99% sequence identity with an HCV NS3 protein or fragment thereof comprising an A39V mutation or a C16S mutation.

In another embodiment, one or more of the polypeptide molecules or fragments thereof of the present invention share between about 100% and 70% sequence identity with p14. In a further embodiment of the invention, one or more of the polypeptide molecules of the invention shares between about 100% and 90% sequence identity with p14. In an embodiment of the invention, one or more of the polypeptide molecules of the invention shares between about 100% and 95% sequence identity with p14. In another embodiment of the invention, one or more of the polypeptides of the invention shares between about 100% and 99% sequence identity with p14.

Match over the length of two sequences may be measured by the Needleman-Wunch algorithm (1970) as described above. The CLUSTAL X program may be used with the following Protein Sequence Parameters:

### Fast Pairwise Alignment Parameters:

K-tuple (word) size: 1
Window size: 5
Scoring method: percentage
Number of top diagonals: 5
Gap penalty: 3

### Multiple Alignment Parameters:

Weight matrix: blosum
Gap opening penalty: 10.0
Gap extension penalty: 0.05
Hydrophilic gaps: On
Hydrophilic residues: GPSNDQERK
Residue-specific gap penalties: On

The compounds of the present invention also include polypeptides that are fused to one another. The compounds of the present invention also include polypeptides that are introduced into host cells.

By the present invention, a polypeptide or fragment thereof may include modifications made by one of ordinary skill in the art. For example, as will be apparent to the skilled art worker, a HCV polypeptide may be modified such as by conservative amino acid changes within the polypeptide sequences of the invention. For example, it is contemplated that a HCV polypeptide or fragments thereof may be modified by conservative amino acid changes that do not diminish the HCV replicase activity of the polypeptide or fragment thereof in the absence of a replicase complex defect inducer.

Conservative changes permit optimization of codon usage, such as for example, if an NS3 protein comprising an A39V mutation or a fragment thereof is to be introduced into a cell or organism. Conservative amino acid changes can be made by substituting one amino acid within one group with another amino acid in the same group. Conservative amino acid changes can also be made by substituting one or more codons with one or more different codons that produce the same amino acids. In this manner, conservative changes are made at the nucleotide level so that the same amino acid is coded for by a different nucleotide sequence. Biologically functional equivalents of the enzymes or fragments thereof of the present invention can have ten or fewer conservative amino acid changes, more preferably seven or fewer conservative amino acid changes, and most preferably five or fewer conservative amino acid changes. The encoding nucleotide sequence will thus have corresponding base substitutions, permitting the nucleotide sequence to encode biologically functional equivalent forms of the enzymes or fragments thereof of the present invention.

It is understood that certain amino acids may be substituted for other amino acids in a polypeptide without appreciable loss of interactive binding capacity with structures such as, for example, antigen-binding regions of antibodies or binding sites on substrate molecules. Certain amino acid sequence substitutions can be made in a polypeptide sequence and, of course, its underlying DNA coding sequence and, nevertheless, a polypeptide with like properties can be obtained. It is thus contemplated that various changes may be made in the polypeptide sequence of the enzymes or fragments thereof of the present invention, or corresponding DNA sequences that encode said polypeptides, without appreciable loss of their biological utility or activity. It is understood that codons capable of coding for such amino acid changes are known in the art.

In making changes to polypeptides of the present invention, the hydropathic index of amino acids may be considered. The importance of the hydropathic amino acid index in conferring interactive biological function on a protein is generally understood in the art (Kyte and Doolittle, J. Mol. Biol. 157, 105-132 (1982)). It is accepted that the relative hydropathic character of amino acids contributes to secondary structure, which in turn defines interaction with other molecules, for example, enzymes, substrates, receptors, DNA, antibodies, antigens, and the like.

Each amino acid has been assigned a hydropathic index on the basis of its hydrophobicity and charge characteristics (Kyte and Doolittle, J. Mol. Biol. 157, 105-132 (1982)); these are isoleucine (+4.5), valine (+4.2), leucine (+3.8), phenylalanine (+2.8), cysteine/cystine (+2.5), methionine (+1.9), alanine (+1.8), glycine (−0.4), threonine (−0.7), serine (−0.8), tryptophan (−0.9), tyrosine (−1.3), proline (−1.6), histidine (−3.2), glutamate (−3.5), glutamine (−3.5), aspartate (−3.5), asparagine (−3.5), lysine (−3.9), and arginine (−4.5).

In making such changes, the substitution of amino acids whose hydropathic indices are within +/−2 is preferred, those within +/−1 are particularly preferred, and those within +/−0.5 are even more particularly preferred.

It is also understood in the art that the substitution of like amino acids can be made effectively on the basis of hydrophilicity. As detailed in U.S. Pat. No. 4,554,101, the following hydrophilicity values have been assigned to amino acid residues: arginine (+3.0), lysine (+3.0), aspartate (+3.0.+/−0.1), glutamate (+3.0.+/−0.1), serine (+0.3), asparagine (+0.2), glutamine (+0.2), glycine (0), threonine (−0.4), proline (−0.5.+/−0.1), alanine (−0.5), histidine (−0.5), cysteine (−1.0), methionine (−1.3), valine (−1.5), leucine (−1.8), isoleucine (−1.8), tyrosine (−2.3), phenylalanine (−2.5), and tryptophan (−3.4).

In making such changes, the substitution of amino acids whose hydrophilicity values are within +/−2 is preferred, those which are within +/−1 are particularly preferred, and those within +/−0.5 are even more particularly preferred. Conservative changes that do not significantly diminish HCV replicase activity of an HCV in the absence of a replicase complex defect inducer are preferred. Such changes may cause less than about a 25% reduction in replicase activity as compared to the activity with no conservative amino acid changes, less than about a 20% reduction in replicase activity as compared to the activity with no conservative amino acid changes, less than about a 15% reduction in replicase activity as compared to the activity with no conservative amino acid changes, less than about a 10% reduction in replicase activity as compared to the activity with no conservative amino acid changes, preferably less than about a 7% reduction in replicase activity as compared to the activity with no conservative amino acid changes, less than about a 5% reduction in replicase activity as compared to the activity with no conservative amino acid changes, less than about a 4% reduction in replicase activity as compared to the activity with no conservative amino acid changes, less than about a 3% reduction in replicase activity as compared to the activity with no conservative amino acid changes, less than about a 2% reduction in replicase activity as compared to the activity with no conservative amino acid changes, less than about a 1% reduction in replicase activity as compared to the activity with no conservative amino acid changes, or no detectable change in replicase activity as compared to the activity with no conservative amino acid changes.

### EXAMPLES

### Example 1

### Resistance Induction

In order to determine the mechanism of action of a test compound, resistance induction experiments are performed. Resistance induction refers to the ability of a clone such as a cell expressing an HCV replicon to become resistant (i.e., to possess the ability to grow) in the presence of a test compound. Identification of mutations in the HCV polyprotein associated with resistance to the test compounds can provide mechanistic information. Resistant clones can also be used for further mechanistic studies. Test compounds, Test A, Test B, Test C, Test D, and Test E, are used. Control compounds include an NS5B nucleoside inhibitor, an NS5B nonnucleoside inhibitor, and an NS3-NS4A protein active site inhibitor.

Huh-7 cells expressing the HCV subgenomic replicon (Accession number AJ242652, SEQ ID NO: 10) are seeded onto 100 mm plates at a density of 1-2×10⁵ cells per plate. On the following day, a test compound is added at a concentration of 10 to 400 fold of its EC₅₀. G418 is used for selection. (see e.g., Lohmann et al., (2001) J. Virol. 75(3), 1487-1499). The concentration of G418 employed for selection is 500 μg/mL to 1 mg/mL. A control plate containing the same concentration of test compound in the absence of G418 is prepared in order to monitor toxicity of the test compounds. Culture medium is changed twice a week until the selection occurs and the resistant colonies are formed. About 6 to about 8 weeks are required to form colonies with a diameter of 3-4 mm. To isolate the resistant colonies, plates are rinsed once with PBS and treated with 1 mL of trypsin—EDTA solution (Invitrogen) at 37° C. for 2 minutes. Individual colonies are removed with a pipette tip and transferred to 24-well plates for further amplification. G418 and the test compound are kept in the culture medium throughout the process to prevent a reversion of resistance.

Colonies that are resistant to various test compounds are isolated and amplified. Resistance of the isolated resistant clones to the test compounds is confirmed by growing the isolated resistant clones in the presence of the test compounds. In addition, the specificity of the resistant clones for the test compounds is determined by incubating the resistant clones in the presence of an NS5B nucleoside inhibitor, and NS5B nonnucleoside inhibitor, or an NS3-NS4A protease inhibitor.

An HCV RNA dot blot assay is used to measure EC₅₀ and/or EC₉₀ of each compound tested in resistant cells as well as wild-type HCV replicon cells. The EC₅₀ is the dose of the test compound that is effective for 50% of the population exposed to the drug or that gives a 50% response in a biological system that is exposed to the drug. The EC₉₀ is the dose of the test compound that is effective for 90% of the population exposed to the drug or that gives a 90% response in a biological system that is exposed to the drug.

Cells expressing the HCV replicon are seeded onto the internal wells of 96-well plates at a density of 7,000 cells per well. One day later, different dilutions of the test compounds are added to corresponding wells and the plates are incubated in a CO₂ incubator for 72 hours. After treatment, the cells are lysed with 70 μl RLN buffer per well (50 mM Tris-HCl [pH8.0], 140 mM NaCl, 1.5 mM MgC₂, 0.5% Nonidet P-40, 1,000 units/mL RNAsin and 1 mM DDT). The total lysate is transferred to a new U bottom 96-well plate and nuclei and cell debris are pelleted with a brief spin (300×g for 3 minutes). 50 μl of supernatant is removed from the well and loaded directly onto manifold wells equipped with Nylon membranes. HCV RNA is hybridized at 58° C. overnight using a labeled HCV RNA probe containing a negative strand sequence complement to the NS5B region. Hybridization buffer is composed of 50% formamide, 5×SSPE, 1% SDS, 5× Denhart's solution, and sheared denatured salmon sperm DNA. Following hybridization, the membrane is washed with washing buffer (0.1×SSC, 0.1% SDS) at room temperature for two hours, then at 65° C. for 30 minutes. The radioactivity in each well is counted on a Microbeta counter and EC₅₀ is calculated based on cpm.

As shown in Table 2, isolated resistant clones are resistant to test compounds, but are sensitive to an NS5B nucleoside inhibitor, an NS5B nonnucleoside inhibitor, and an NS3-NS4A protease inhibitor.

TABLE 2

| | | Average |
|---|---|---|
| Resistant | | fold |
| clone | Compound | change in EC₅₀ |
| Clone | Test B | 11.6 |
| resistant to | Test A | 11.7 |
| Test A | Nonnucleoside NS5B | 1.5 |
| | inhibitor | |
| | NS3-NS4A protease | 1.2 |
| | inhibitor | |
| | Nucleoside NS5B inhibitor | 0.5 |
| Clone | Test E | 13.0 |
| resistant to | Test B | 14.2 |
| Test B | Test F | 7.6 |
| | Nonnucleoside NS5B | 1.3 |
| | inhibitor | |
| | Nucleoside NS5B inhibitor | 1.5 |

Table 2 demonstrates that clones identified in the resistance induction experiment are resistant to the test compounds but not to other types of HCV inhibitors. Also, the clones identified in the resistance induction experiment are resistant to test compounds other than the one for which they were selected. For example, a clone isolated as resistant to Test B is also resistant to Test A.

As shown in Table 3, clones resistant to an NS5B nucleoside inhibitor, and NS5B nonnucleoside inhibitor or an NS3-NS4A protease inhibitor, are resistant to the inhibitor for which they were selected but not to other classes of inhibitors including test compounds.

TABLE 3

| | | Average |
|---|---|---|
| | | fold |
| Resistant Clone | Compound | change in EC₅₀ |
| Clone resistant to | Test E | 1.0 |
| Nonnucleoside | Test A | 0.7 |
| NS5B inhibitor | Nonnucleoside NS5B | >36.1 |
| | inhibitor | |
| | NS3-NS4A protease | 2.2 |
| | inhibitor | |
| | Nucleoside NS5B inhibitor | 0.4 |
| Clone resistant to | Test E | 1.1 |
| NS3-NS4A | Test A | 0.9 |
| protease inhibitor | Nonnucleoside NS5B | 1.3 |
| | inhibitor | |
| | NS3-NS4A protease | >33.8 |
| | inhibitor | |
| | Nucleoside NS5B inhibitor | 0.5 |
| Clone resistant to | Test B | 0.7 |
| Nucleoside NS5B | Nonnucleoside NS5B | 4.0 |
| inhibitor | inhibitor | |
| | NS3-NS4A protease | 1.2 |
| | inhibitor | |
| | Nucleoside NS5B inhibitor | 23.3 |

As illustrated in Table 3, clones resistant to other classes of inhibitors remain sensitive to the test compounds. Also, a greater change in EC₅₀ is observed with other classes of inhibitors compared to the test compounds.

The resistance induction experiments suggest that the test compounds inhibit HCV by a mechanism different from that of known nonnucleoside NS5B inhibitors, NS3-NS4A protease inhibitors, and nucleoside NS5B inhibitors.

Other replicons, including those of SEQ ID NOs: 8, 9, 11-27 are used with various test compounds to yield parallel results.

### Example 2

### Genotypic Analysis of Resistant Clones

The clones that are resistant to test compounds are subjected to a genotypic analysis to determine the mutation conferring the resistant phenotype. To find the consensus mutations in resistant HCV genomes, total RNA is extracted from several resistant cell lines cultured in 75 mL flasks with Trio reagent (Invitrogen). The cDNA complement to the HCV coding region of the nonstructural protein ranging from NS3 to NS5B is synthesized by using SuperScript™ First-Strand synthesis system (Invitrogen). A reverse primer used in cDNA synthesis is (SEQ ID NO:28) 5′ACTTGATCTGCAGAGAGGCCAGTATCAG 3′ 7962. PCR amplification of HCV cDNA is done with Advantage™-GC2 PCR kit (BD Biosciences). PCR products of resistant HCV RNA and a parental HCV RNA are sequenced and the sequences aligned in order to discern the consensus mutations.

An A39V mutation is present in the NS3 protein all of the resistant clones induced with Test A and B. The A39V mutation is present in the clones resistant to the test compounds and is not found in clones resistant to the other classes of HCV inhibitors.

The three-dimensional structure of the protease catalytic domain of NS3 has been determined by X-ray crystallography, with and without a cofactor peptide from NS4A. These structures reveal very strong structural homology to chymotrypsin-like serine protease domains with the canonical catalytic triad comprising Ser-139, His-57, and Asp-81. The N-terminal 28 amino acids of NS3 are unstructured in the absence of NS4A, while in the presence of NS4A peptide this region adopts β-strand and α-helix secondary structures. The co-crystal structure reveal that the NS4A peptide is inserted into, and partially buried by, adjacent β-strands of NS3. Local rearrangements near the protease active site also occur as a result of NS4A binding, and these are thought to render the protease more catalytically active. Near the N-terminus of NS3 is an α-helix spanning residues 13-21 (α-helix 0) that appears to be stabilized by the NS4A peptide. The external face of this helix is very hydrophobic and consists entirely of branched aliphatic residues. Based on the published structures of NS3-NS4A, the A39V mutation is very close to the NS3-NS4A structural interface and is not in the active site of the NS3 protease.

### Example 3

### Reverse Genetics to Confirm Role of Resistant Phenotype

Transient transfection assays are performed to confirm the role of the A39V mutation in conferring resistance to the test compounds. Plasmid pFKI341-PI-Luc/NS3-3′/A39V (resistant clone) and pFKI341-PI-Luc/NS3-3′ (wild-type clone), are linearized with AseI and ScaI sequentially. These clones comprise Accession number AJ242652 (SEQ ID NO: 10) with an A39V mutation and AJ242652 (SEQ ID NO: 10), respectively, each with a luciferase reporter gene. After extraction with phenol-chloroform and ethanol precipitation, DNA is dissolved in RNAse-free deionized water. Five μg of linearized DNA is used for in vitro transcription reactions containing 80 mM HEPES (pH 7.5), 12 mM MgCl₂, 2 mM spermidine, 40 mM DTT, 25 mM each NTP, 100 units of RNAsin (Promega) and 80 Units T7-RNA-polymerase (Promega). After 2 hours at 37° C., an additional 40 units of T7-RNA-polymerase are added and the reaction mixture is incubated for another 2 hours. Transcription is terminated by the addition of 2 units of RNAse-free DNAse (Promega) per μg of plasmid DNA and 1 hour incubation at 37° C. After one extraction with acidic phenol and chloroform, RNA is precipitated with isopropanol and dissolved in RNAse-free water. The concentration is determined by measurement of the optical density at 260 nm and the RNA integrity is confirmed by denaturing agarose gel electrophoresis. Five μg of in vitro transcribed RNA is mixed with 400 μl of a suspension of 10⁷ Huh-7 cells per ml. Electroporation conditions are 950 μF and 270 V using a Gene pulser system (Bio-Rad) and a cuvette with a gap width of 0.4 cm (Bio-Rad). Cells are immediately transferred to 8 ml of complete DMEM medium and seeded in 96-well plate with 10,000 cells/well. 24 hours after transfection, test compounds are added. Lucerifase assays are performed 72 hours after addition of test compounds by Ultra-High Sensitivity Luminescence Reporter Gene Assay System (Perkin Elmer) according the to manufacturer's instructions.

Results are shown in Table 4:

TABLE 4

| | Luciferase | | Luciferase | | | |
|---|---|---|---|---|---|---|
| | activity | | activity | | A39V | |
| | Wild type | | A39V mutant | | mutant/wild- | |
| | replicon | | replicon | | type | |
| | EC₅₀ | EC₉₀ | EC₅₀ | EC₉₀ | EC₅₀ | EC₉₀ |
| Compound | (μM) | (μM) | (μM) | (μM) | (μM) | (μM) |
| Test E | 0.007 | 0.046 | 0.155 | 0.632 | 22.8 | 13.7 |
| Nonnucleoside NS5B | 0.244 | 1.338 | 0.311 | 2.020 | 1.3 | 1.5 |
| inhibitor | | | | | | |
| NS3-NS4A protease | 1.750 | 5.718 | 2.270 | 7.052 | 1.3 | 1.2 |
| inhibitor | | | | | | |
| Nucleoside NS5B | 0.216 | 0.849 | 0.207 | 0.831 | 1.0 | 1.0 |
| inhibitor | | | | | | |
| Nucleoside NS5B | 0.313 | 1.621 | 0.299 | 1.126 | 1.0 | 0.7 |
| inhibitor | | | | | | |

The above data show a 20-fold shift in the potency of the Test E compound in A39V mutant replicons compared to wild-type replicons. The potency of other classes of inhibitors (e.g., nonnucleoside NS5B inhibitor, NS3-NS4A protease inhibitor, and nucleoside NS5B inhibitor) remain unchanged. These data are consistent with a correlation between the A39V mutation and resistance in resistant cell lines.

The shift in potency of the test compound resistant clones compared to wild-type clones is usually about 10-fold. The A39V mutation is unique to the test compounds and is not found in the parent clones or resistant clones obtained with other classes of inhibitors. Reverse genetics studies confirm that the A39V mutation confers resistance of the cell lines to the test compounds.

While the A39V mutation is clearly related to the resistant phenotype selected in these experiments, it is noted that A39V may not be the only mutation associated with the mechanism of action described below. In additional data not shown herein, other test compounds having the same mechanism of action as Test A and B produce different mutations in the HCV replicon RNA.

### Example 4

### Reverse Genetics to Confirm Role of Resistant Phenotype

Mutations other than the A39V mutation are prepared and identified as in Examples 1 and 2. Transient transfection assays are performed to confirm the role of mutations other than A39V in conferring resistance to the test compounds. Plasmid pFKI341-PI-Luc/NS3-3′/A39V (resistant clone) and pFKI341-PI-Luc/NS3-3′ (wild-type clone), are linearized with AseI and ScaI sequentially. These resistant and wild-type clones comprise Accession number AJ242652 (SEQ ID NO: 10) with a mutation and AJ242652 (SEQ ID NO: 10), respectively, each with a luciferase reporter gene. After extraction with phenol-chloroform and ethanol precipitation, DNA is dissolved in RNAse-free deionized water. Five μg of linearized DNA is used for in vitro transcription reactions containing 80 mM HEPES (pH 7.5), 12 mM MgCl₂, 2 mM spermidine, 40 mM DTT, 25 mM each NTP, 100 units of RNAsin (Promega) and 80 Units T7-RNA-polymerase (Promega). After 2 hours at 37° C., an additional 40 units of T7-RNA-polymerase are added and the reaction mixture is incubated for another 2 hours. Transcription is terminated by the addition of 2 units of RNAse-free DNAse (Promega) per μg of plasmid DNA and 1 hour incubation at 37° C. After one extraction with acidic phenol and chloroform, RNA is precipitated with isopropanol and dissolved in RNAse-free water. The concentration is determined by measurement of the optical density at 260 nm and the RNA integrity is confirmed by denaturing agarose gel electrophoresis. Five μg of in vitro transcribed RNA is mixed with 400 μl of a suspension of 10⁷ Huh-7 cells per ml. Electroporation conditions are 950 μF and 270 V using a Gene pulser system (Bio-Rad) and a cuvette with a gap width of 0.4 cm (Bio-Rad). Cells are immediately transferred to 8 ml of complete DMEM medium and seeded in 96-well plate with 10,000 cells/well. 24 hours after transfection, test compounds are added. Lucerifase assays are performed 72 hours after addition of test compounds by Ultra-High Sensitivity Luminescence Reporter Gene Assay System (Perkin Elmer) according the to manufacturer's instructions.

A shift in the potency of test compounds in mutant replicons is observed as compared to in wild-type replicons. The potency of other classes of inhibitors (e.g., nonnucleoside NS5B inhibitor, NS3-NS4A protease inhibitor, and nucleoside NS5B inhibitor) remain unchanged. These data are consistent with a correlation between a mutation and resistance in resistant cell lines.

### Example 5

### Mechanism of Action of the Test Compounds: In Vitro and In Vivo RNA Synthesis

The effect of the test compounds on nascent RNA synthesis is studied in vitro to determine if the test compounds directly affect the activity of replication complexes in vitro. To isolate replicase complexes, Huh-7 cells expressing the HCV replicon are washed with 1×PBS, re-suspended in cold hypotonic buffer (10 mM Tri-HCl, pH 7.8, mM NaCl), and put on ice for 20 minutes. The swelled cells are disrupted using a dounce homogenizer. The mix is centrifuged at 900×g for 5 minutes at 4° C. The supernatant is transferred to a fresh tube and centrifuged at 15000×g for 25 minutes at 4° C. The pellet, which contains the membrane fraction, is re-suspended in storage buffer (hypotonic buffer with 15% glycerol), and stored at −80° C.

100,000 cells expressing the HCV replicon as well as equal number of Huh-7 cells are seeded onto wells of 6-well plates and incubated for 72 hours. The wells are washed with starvation medium (DMEM medium lacking phosphate) supplemented with 5% dialyzed FBS, 1/20 of the normal concentration of phosphate). The cells are phosphate starved by incubating the cells in CO₂ incubator at 37° C. for 1 hour. Dilutions of 100 μM, 31.6 μM, 10 μM, and 3.16 μM Test G containing 10 ug/mL actinomycin D are made. Cells are incubated for an additional hour with a fresh medium above containing 10 ug/mL actinomycin D and the Test G compound, 1 ml/well. 166 μCi ³²P orthophosphate diluted in culture medium is added and the cells are incubated at 37° C. for 3 to 12 hours. The cells are washed with PBS and lysed with 1 ml of Trizol. The lysate is extracted with chloroform and the RNA is then ethanol precipitated. The RNA is dissolved with 20 μl formimide, 9 μl Gibco RNAse-free water, 2 μl 10× running buffer, and 7 μl formadehyde are added sequentially. The RNA is denatured by heating the sample at 65° C. for 15 minutes. Samples are loaded onto a 1.0% agarose gel. After electrophoresis, the gel is soaked in 10% glacial acetic acid for 20 minutes, then in ethanol for another 20 minutes, dried and exposed to an X-ray film.

The test compound (Test G) reduces HCV RNA levels in a dose-dependent manner when the label is present throughout the course of the experiment. The test compounds affect synthesis of HCV RNA. RNA levels, however, are not reduced to background levels even in the presence of high levels of test compound, which result is consistent with the NS5B polymerase being active and the test compounds not affecting the activity of NS5B. A nucleoside NS5B inhibitor, in contrast, completely inhibits RNA synthesis under these conditions.

A reduction in RNA synthesis in the presence of the test compound is also observed when RNA labeling is conducted four hours post treatment with the Test G. This experiment is essentially identical to the previous experiment except for the timing of labeling. In contrast to the previous experiment, complete reduction in RNA synthesis is observed, which is consistent with the test compound blocking RNA synthesis prior to elongation.

RNA synthesis proceeds as a two-step process: initiation and elongation. In initiation, an initiated template RNA is formed in which only a portion of the newly synthesized positive or negative strand RNA is made using a minus or plus strand template. In elongation, the remainder of the positive or negative strand RNA is synthesized. Because incomplete RNA reduction is observed when RNA is labeled immediately after incubation with the test compound, and complete RNA reduction is observed when labeling is performed 4 hours after incubation with the test compound, the test compounds block RNA synthesis prior to elongation. That is, previously initiated RNAs are elongated, but no new initiation occurs. This is because elongation will not occur efficiently without initiation. The lack of elongation strongly suggests that the block in HCV replication occurs prior to elongation, e.g., in initiation or prior to initiation such as in replicase complex formation.

The effect of the test compounds on the activity of replicase complexes in vitro and in vivo is also examined. In the in vitro assay, replicase complexes are isolated and in vitro RNA synthesis is performed in the presence of a test compound or other inhibitor. In the in vivo replicase complex assay, cells expressing the HCV replicon are treated with test compound before the preparation of replicase complexes and RNA synthesis. Huh-7 cells expressing the HCV replicon are seeded onto 100 mm dishes and cultured to confluence. The cells are incubated in the medium containing different concentrations of test compound, or no test compound, for 4 to 16 hours. After treatment, cells are washed once with PBS and frozen at −80° C. overnight. The frozen cells are put on ice and are lysed in 1 mL of hypotonic buffer (10 mM Tris-HCl [pH 8.0], 10 mM sodium acetate, 1.5 mM MgCl₂) and passed through a 21G2-gauge needle 30 times. Nuclei and unbroken cells are removed by centrifugation at 600×g for 6 minutes in a microcentrifuge at 4° C. The supernatant is centrifuged at 14,000 rpm in a microcentrifuge at 4° C. for 20 minutes. The pellet contains replicase complex-enriched membrane and is re-suspended in 6 μl hypotonic buffer per plate.

In the in vitro RNA synthesis experiment, in vitro activity is studied by performing in vitro RNA synthesis with isolated replicase complexes in the presence of the Test B compound. The Test B compound has little effect on double-stranded or single-stranded RNA synthesis. This result is consistent with a conclusion that the test compounds do not inhibit NS5B polymerase activity.

In the in vivo RNA synthesis experiment, cells expressing the HCV replicon are incubated with the test compound for 4 hours prior to isolation of the replicase complexes. Isolated replicase complexes are used to perform in vitro RNA synthesis. Test G reduces the level of both single-stranded and double-stranded RNA produced from isolated replicase complexes. In summary, the test compounds do not block the activity of pre-formed replicase complexes, and thus do not block NS5B polymerase activity. The test compounds decrease replicase complex activity when cells are grown in the presence of the test compound. Taken together, these results suggest that the test compounds inhibit the production of functional replicase complexes.

### Example 6

### Mechanism of Action: Effects on Viral Protein Composition

Based on the results above, it was hypothesized that the test compounds inhibit the production of functional replicase complexes. To test this hypothesis, the effects of the test compounds on viral polyprotein synthesis and processing are studied. The effect of the test compounds on NS5A synthesis and processing is examined using immunoprecipitation of replication complexes with an anti-NS5A antibody. Cells expressing no HCV replicon, HCV replicon in the absence of test compound, and HCV replicon in the presence of the test compound are employed.

In this experiment, 5×10⁵ cells/well of Huh-7 cells expressing HCV replicon, isolated replicase complex, isolated HCV polyprotein or control Huh-7 cells are seeded onto a 6 well plate. The next day (16 to 24 hours, when cells grow to more than 90% confluency), the medium is removed, and the cells are washed three times with PBS. FCS-free, Met- and Cys-free medium is added, and the cells are incubated for 1 hour. The test compounds are added to the medium at the same time for sample treatment. The medium is then replaced with Met- and Cys-free medium plus EXPRESS Protein Labeling Mix, [³⁵S]-, 7 mCi (259 MBq), (PerkinElmer, Inc, NEG072007MC) at the concentration of 150 μCi/ml and cells are incubated for 5 to 24 hours at 37° C. Experiments are done either as steady-state labeling experiments or pulse-chase experiments in which a pulse of labeled amino acids is followed by a chase of unlabeled amino acids.

After the experiment is complete, the medium is removed and the cells washed three times with PBS. Cells are lysed using 0.6 ml/well 1×NPB buffer (50 mM Tris-pH 7.5, 150 mM NaCl, 1% Sodium Deoxycholate, 0.1% SDS) plus protease inhibitors (Complete, Mini, EDTA-free. Roche, Cat # 1836170). The cells are centrifuged at 14000 rpm (20817×g) at 4° C. for 15 minutes. The supernatant is transferred to fresh tube and used for immunoprecipitation or stored at −80° C.

For detection, 5 μl of anti-NS5A antibody is pre-bound to protein A beads (Invitrogen, Cat # 15918-014) at 4° C. for 1 hour. The bound antibody is then added to 0.6 ml of lysate and rotated at 4° C. overnight. The beads are washed four times with RIPA (PBS, 1% triton X-100, 0.5% sodium deoxycholate) with protease inhibitors. The proteins are resolved on a 7.5% Tris HCl gel. The gel is fixed and followed by autoradiography.

For the Test H compound, no apparent reduction in the NS5A protein is observed in the presence of the test compound in a pulse-chase experiment. A steady-state labeling experiment is also performed. In the presence of the test compound (Test B), production of levels of NS5A comparable to those of the no inhibitor control are produced, and no buildup of NS5A precursors is observed. In the presence of an NS3 protease inhibitor, reduced levels of NS5A are observed compared to the no inhibitor control and some build-up of NS5A precursors is observed. This build up in precursors occurs because the NS3 protease is responsible for NS5A/NS5B cleavage. In contrast to the protease inhibitors, the test compounds have no significant effect on NS5A processing and therefore the test compounds do not inhibit viral serine protease enzymatic activity.

All other viral protein levels after treatment with the test compound or other classes of inhibitors for 16 hours is determined by immunoblotting. In this experiment, 5×10⁶ cells/dish of cells expressing the HCV replicon are seeded onto 150 mm dishes. 48 hours later when cells grew to more than 80% confluency, test compounds are added and the cells are incubated for 2 to 24 hours. Cells are collected and lysed in 1.5 ml 1×NPB buffer with protease inhibitors. Cells are centrifuged at 14000 rpm (20817×g) at 4° C. for 15 minutes or the lysate is passed through a QIAshredder (Qiagen, Cat 79654). The supernatant of cell lysate is transferred to a fresh tube and used for immunoprecipitation or stored at −80° C.

For detection, 4 μl of anti-NS3 (Anogen, Cat. MO-140018K) or 4 μl of anti-NS4A (Biodesign, Cat. C8A236M) is pre-bound to protein A beads at 4° C. for 1 hour. The bound antibody is added to the cell lysate and incubated at 4° C. overnight, rocking gently. The beads are washed four times with PBS plus 0.05% Tween 20 and protease inhibitors. The proteins are resolved on a 4 to 20% or a 10 to 20% gradient Tris HCl gel. The sample in the gel is transferred to a membrane (Immun-Blot Immun-Blot PVDF/Filter Paper Sandwich, 8.5×13.5 cm, 50, Bio-Rad, Cat 162-0239) according to the manufacturer's instructions.

The membrane is blocked in 5% dry milk in PBST (PBS plus 0.05% Tween 20) at room temperature for 1 to 2 hours. The blocking solution is removed and the first antibody solution (anti-NS4A (1:2000), anti-NS3 (1:10000), anti-NS5A (1:5000), anti-NS4B (1:15000) in 2.5% dry milk in PBST) is added. The membrane is rocked gently at room temperature for 2 hours. The membrane is washed three times for 15 minutes/wash in PBST. Secondary antibody (1:5000) in 2.5% dry milk in PBST is then added, and the membrane is incubated with the antibody at room temperature for 1 hour. The membrane is washed twice with PBST and twice with PBS. The membrane is developed with ECL plus (Amersham Biosciences, Cat # RPN2133) and exposed to X-ray film.

The protein levels after treatment with the Test E compound or other classes of inhibitors for 16 hours are determined by immunoblotting. The Test E compound selectively reduces the amount of NS3 and NS4A. This selective reduction of NS3 and NS4A is not observed for the other classes of inhibitors.

In this experiment, Huh-7 cells expressing the HCV replicon are treated with the Test E inhibitor for 8 hours. The viral proteins are then immunoprecipitated with anti-NS4A antibodies. Immunoblotting is performed with anti-NS3 or anti-NS4A antibodies. The NS3 level is reduced in the presence of the test compounds. Interestingly, a 14 KDa protein band (p14) is detected in cells treated with the test compound. A very long exposure shows a small amount of p14 in both the untreated cells and the NS5B inhibitor treated cells; however, the p14 band is greatly enhanced in the presence of the test compounds. An underexposure of the immunoblot shows a decrease in NS4A upon treatment with all of the inhibitors. The large enhancement of the p14 band in the presence of the test compounds suggests that this product may be related to replicase complex inhibition in the presence of the test compounds.

The dose-dependence of the p14 band intensity for Test E is studied to determine if the intensity of the p14 band is directly related to the amount of test compound added. This experiment is performed in the same manner as the previous experiment (i.e., immunoprecipitation with an anti-NS4A antibody followed by immunoblotting with anti-NS4A or anti-NS3) except the Test E concentration is varied from 10 μM, 2 μM, 0.4 μM and 0.08 μM. It is observed that p14 accumulates in a dose-dependent manner upon treatment with Test E. Also, the level of NS3 is reduced in a dose-dependent manner.

Treatment with a higher concentration and a longer duration of the Test E compound results in a dose-dependent reduction in the amount of NS4A and the production of a band slightly larger than NS4A (NS4A*). Thus, miscleavage of NS4A also appears to result in the formation of a double NS4A band in the presence of the test compounds. Without being bound by any particular theory, it is believed that the NS4A band is derived from cleavage near the C-terminal end of NS3 and thus comprises NS4A plus a few amino acids of NS3.

### Example 7

### Mechanism of Action: Further Characterization of the NS4A Product

Experiments are undertaken to investigate the affinity and binding specificity of acylthiourea compounds to NS4A. The full length of NS4A (54 amino acids) is synthesized, and characterized with mass spectral analysis and HPLC profiling. An NS4A sample is then dissolved in 100% dimethylsulphoxide (DMSO) at a concentration of 2 μg/μl and subjected to a 10-20% SDS-PAGE with a reducing reagent. A 6 kDa protein band, consistent with the size of NS4A, is detected in the gel following a Coomassie blue staining. The NS4A identity of this band is further confirmed by a Western blot with monoclonal anti-NS4A antibody.

To determine whether acylthiourea compounds bind to NS4A, a photo-affinity labeling is carried out, in which an [³H] labeled photo-reactive azidoacylthiourea, 1-(5-Azido-benzofuran-2-carbonyl)-3-(4-pentyloxy-3-trifluoromethyl-phenyl)-thiourea, is mixed with synthetic NS4A at various molar ratios. The mixture is incubated at 31° C. for one hour. Samples are then illuminated by a UV lamp (254 nm wavelength) at room temperature for 7 minutes. After denaturing with equal volume of Laemmli buffer containing 5% mercaptoethanol at 99° C. for 5 minutes, samples are separated on a 10-20% SDS-PAGE. After electrophoresis, the gel is fixed with 30% methanol—10% acetic acid overnight. Then the gel is treated with 3H enhancer solution (PerkinElmer) at room temperature for one hour, and subsequently with water for 30 minutes, and dried under vacuum.

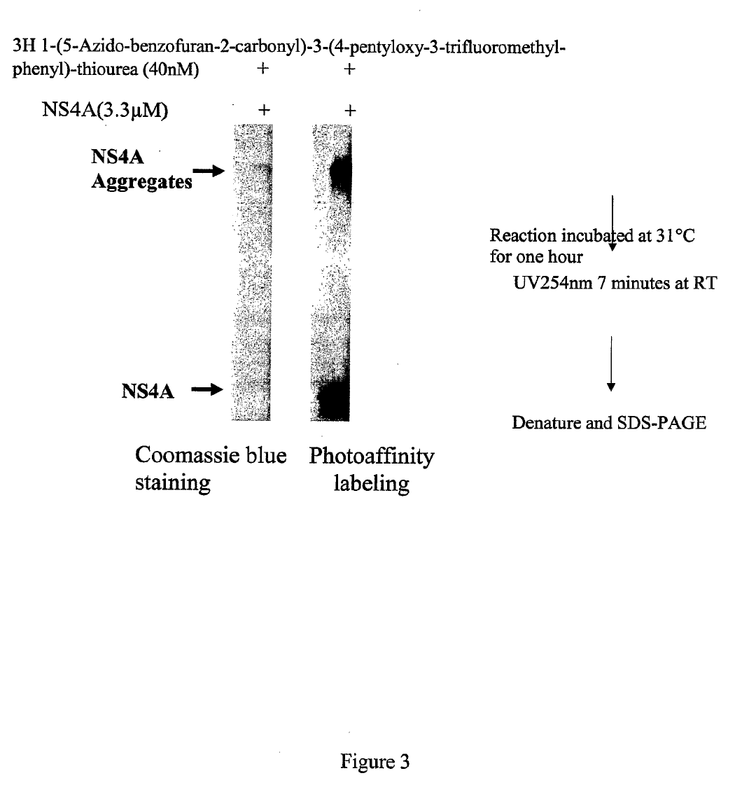

Following the photolysis of the [³H] labeled 1-(5-Azido-benzofuran-2-carbonyl)-3-(4-pentyloxy-3-trifluoromethyl-phenyl)-thiourea incubated with synthetic NS4A, two radioactive bands are shown in the gel (FIG. 3). One band corresponds to NS4A monomer, which is also detectable with Coomassie blue staining (FIG. 3). The second band is located toward the top of the gel, suggesting a high molecular weight. Without being bound by any particular theory, it is believed that this band may be composed largely of polymer form or aggregates of NS4A. At the same time, full-length NS3 and NS3 protease domain is included in this binding assay. Though the molar ratios for these two proteins are the same as that of NS4A (3.3 μM), only weak binding signals are observed for both full-length NS3 and NS3 protease domain.

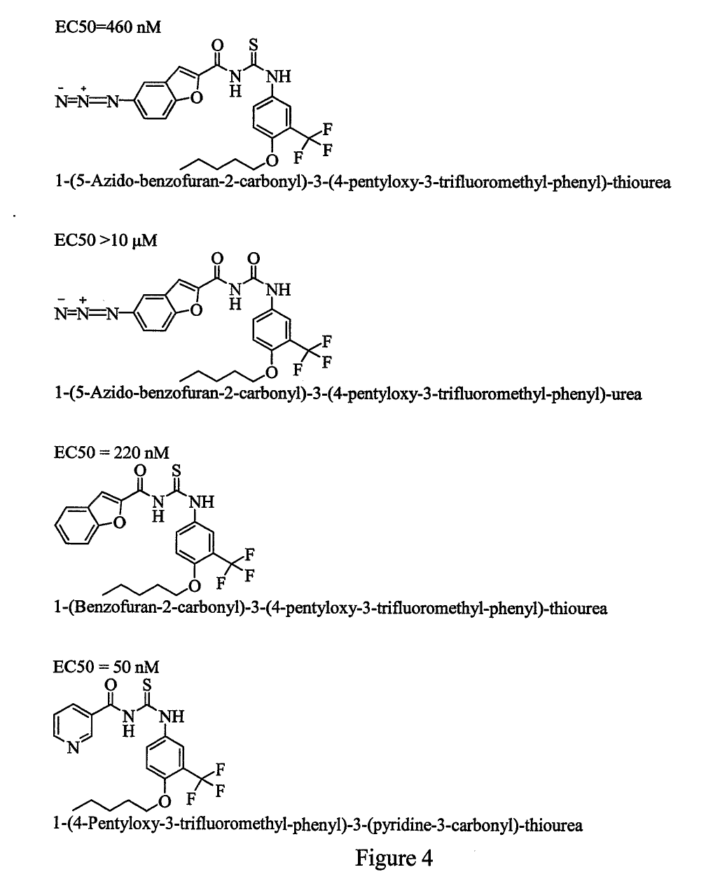

To study the binding specificity of [³H] labeled 1-(5-Azido-benzofuran-2-carbonyl)-3-(4-pentyloxy-3-trifluoromethyl-phenyl)-thiourea to synthetic NS4A, several structurally related compounds are used in the following competition assays. The structures of those compounds are shown in FIG. 4. Non-labeled 1-(5-Azido-benzofuran-2-carbonyl)-3-(4-pentyloxy-3-trifluoromethyl-phenyl)-thiourea shares the same structure with [³H] labeled 1-(5-Azido-benzofuran-2-carbonyl)-3-(4-pentyloxy-3-trifluoromethyl-phenyl)-thiourea. 1-(5-Azido-benzofuran-2-carbonyl)-3-(4-pentyloxy-3-trifluoromethyl-phenyl)-urea differs in structure from 1-(5-Azido-benzofuran-2-carbonyl)-3-(4-pentyloxy-3-trifluoromethyl-phenyl)-thiourea in that 1-(5-Azido-benzofuran-2-carbonyl)-3-(4-pentyloxy-3-trifluoromethyl-phenyl)-urea has a carbonyl group where 1-(5-Azido-benzofuran-2-carbonyl)-3-(4-pentyloxy-3-trifluoromethyl-phenyl)-thiourea has a thiocarbonyl group. 1-(5-Azido-benzofuran-2-carbonyl)-3-(4-pentyloxy-3-trifluoromethyl-phenyl)-urea does not exhibit any anti-HCV activity in replicon cells. 1-(Benzofuran-2-carbonyl)-3-(4-pentyloxy-3-trifluoromethyl-phenyl)-thiourea differs in structure from 1-(5-Azido-benzofuran-2-carbonyl)-3-(4-pentyloxy-3-trifluoromethyl-phenyl)-thiourea in that 1-(Benzofuran-2-carbonyl)-3-(4-pentyloxy-3-trifluoromethyl-phenyl)-thiourea has a hydrogen where 1-(5-Azido-benzofuran-2-carbonyl)-3-(4-pentyloxy-3-trifluoromethyl-phenyl)-thiourea has an azido group. 1-(Benzofuran-2-carbonyl)-3-(4-pentyloxy-3-trifluoromethyl-phenyl)-thiourea is not photo reactive. 1-(4-Pentyloxy-3-trifluoromethyl-phenyl)-3-(pyridine-3-carbonyl)-thiourea differs in structure from 1-(5-Azido-benzofuran-2-carbonyl)-3-(4-pentyloxy-3-trifluoromethyl-phenyl)-thiourea in that it has a pyridinyl group where 1-(5-Azido-benzofuran-2-carbonyl)-3-(4-pentyloxy-3-trifluoromethyl-phenyl)-thiourea has an azidobenzofurano group.

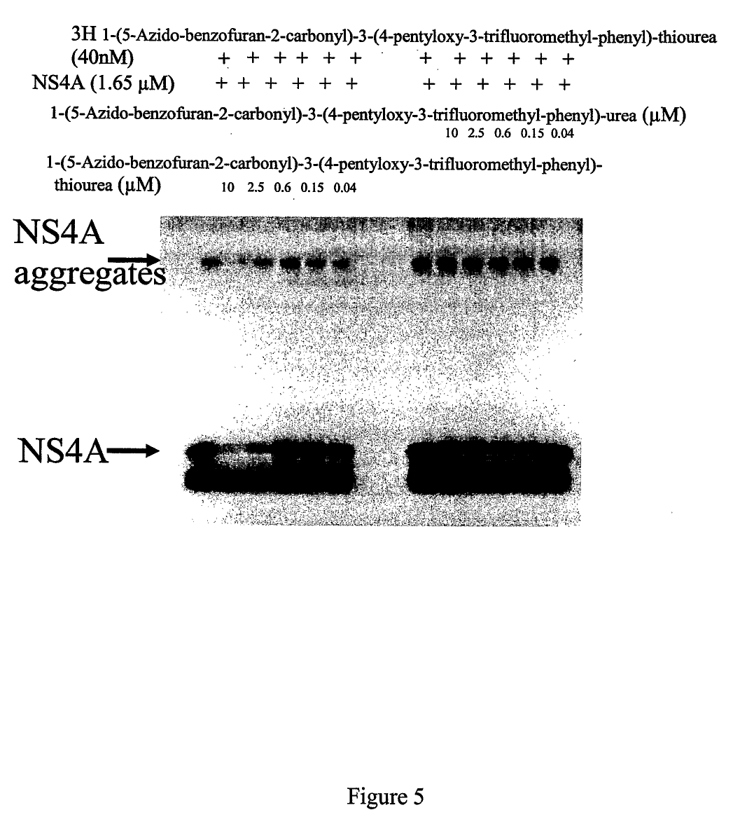

As shown in FIG. 5, acylthioureas that exhibit anti-HCV activities in replicon cells effectively compete with [³H] labeled 1-(5-Azido-benzofuran-2-carbonyl)-3-(4-pentyloxy-3-trifluoromethyl-phenyl)-thiourea for NS4A binding, regardless of whether they have a different heteroaryl group (1-(4-Pentyloxy-3-trifluoromethyl-phenyl)-3-(pyridine-3-carbonyl)-thiourea) or no azido group (1-(Benzofuran-2-carbonyl)-3-(4-pentyloxy-3-trifluoromethyl-phenyl)-thiourea). However, the replacement of thiocarbonyl with carbonyl in the core region (1-(5-Azido-benzofuran-2-carbonyl)-3-(4-pentyloxy-3-trifluoromethyl-phenyl)-urea), which has been demonstrated to affect the compound's anti-HCV activity, appears to abolish this competition capacity. Without being bound by any particular theory, these results suggest that acrylthioureas efficiently bind to synthetic NS4A, and the “core region” of these compounds plays a role in NS4A binding.

### Example 8

### Detection of NS4A* by In Vitro Transcription and Translation

Two plasmids that encode NS3-4A are used for in vitro transcription. One plasmid has protease activity and the other lacks protease activity due to an S139A mutation in the active site of NS3. RNA templates are made from the plasmid encoding the wild type NS3-4A and the plasmid encoding NS3-4A with an S139A mutation. After RNA is made in vitro by T7 RNA polymerase, RNA is purified and used as the template for translation of NS3-4A protein by rabbit reticulocyte lysates. Reactions are set up with no test compound added to RNA made from NS3-4A with an S139A mutation and 0, 3.16 or 10 μM of Test E compound added to RNA made from wild type NS3-4A. At the end of reaction, half of each product is immunoprecipitated with anti-NS3 and the other half is immunoprecipitated with anti-NS4A. After resolving the immunoprecipitated products on SDS/PAGE, immunoblotting is performed with anti-NS4A. Three distinct bands are observed to migrate at three distinct positions corresponding to NS4A, NS4A* and NS3-4A.

### Example 9

### Replicon, Replicase Complex, and Viral Protein Assays

### Replicon

Cells expressing the HCV replicon are seeded onto the internal wells of 96-well plates at a density of 7,000 cells per well. One day later, different dilutions of the test compounds are added to corresponding wells and the plates are incubated in a CO₂ incubator for 72 hours. After treatment, the cells are lysed with 70 μl RLN buffer per well (50 mM Tris-HCl [pH8.0], 140 mM NaCl, 1.5 mM MgCl₂, 0.5% Nonidet P-40, 1,000 units/mL RNAsin and 1 mM DDT). The total lysate is transferred to a new U bottom 96-well plate and nuclei and cell debris are pelleted with a brief spin (300×g for 3 minutes). 50 μl of supernatant is removed from the well and loaded directly onto manifold wells equipped with Nylon membranes. HCV RNA is hybridized at 58° C. overnight using a labeled HCV RNA probe containing a negative strand sequence complement to the NS5B region. Hybridization buffer is composed of 50% formamide, 5×SSPE, 1% SDS, 5× Denhart's solution, and sheared denatured salmon sperm DNA. Following hybridization, the membrane is washed with washing buffer (0.1×SSC, 0.1% SDS) at room temperature for two hours, then at 65° C. for 30 minutes. The radioactivity in each well is counted on a Microbeta counter and EC₅₀ is calculated based on cpm. Alternatively, HCV replicon cells are trypsinized for 5-10 minutes in a CO₂ incubator. Then, cells are placed in a 15 mL tube, and are spun at 1000 rpm for 4 minutes and trypsin solution is removed. Cell pellets are washed once with PBS and spun 4 minutes at 1000 rpm.

Trypsinization and wash steps, supra, are conducted efficiently to minimize cell death. PBS is removed and cells are resuspended in 4 mL fresh PBS and cells are seeded at a dilution of 5000 cells per well to 96 well plates. Test compounds are diluted in 96 well U bottom plates with DMEM (high glucose) (BRL, Catalog #21063) supplemented with 10% FBS, L-glutamine, and non-essential amino acid. 100 uL of test compound is loaded into each well. Media alone is used as a control. An interferon control is also prepared. Six dilutions are made at final concentrations ranging from 200 IU/mL to 0.62 IU/mL. Cells are grown for approximately 72 hours.

After growth, cell culture medium is dumped from the wells without touching the cell layer. 200 uL of 1×PBS, pH 7.4 is added slowly to all wells without touching cultured cells. The plate is shaken for 1 minute and PBS is completely removed. Residual PBS solution may be removed by tapping the plate on a piece of paper. Cells are fixed by using 200 uL per well of chilled (−20° C.) methanol:acetone (1:1) and placing the plates at −20° C. for at least one hour.

The fixing solution is dumped and plates are air dried completely. Wells are blocked with 200 uL of PBS-10% FBS for 1 hour at room temperature. Blocking solution is removed, and 100 uL of primary antibody, rabbit anti-NPTII (Cortex Biochem., Catalog #CR1112RP) diluted 1:1000 in PBS-10% FBS. Plates are incubated for 45 minutes to 1 hour at room temperature.

Wells are washed 6 times with PBS-0.05% Tween-20. 100 uL of secondary antibody, HUR-conjugated goat anti-rabbit (Biosource International) is diluted 1:8000 in PBS-10% FBS and added to each well, and the plates are incubated at room temperature for 30-45 minutes. The plates are washed 6 times with PBS-0.05% Tween-20.

100 uL of development solution (TMB one-step substrate system) is added and plates are developed in the dark for 10 to 30 minutes. The reaction is stopped using 100 uL of 0.2N HCl. OD₄₅₀ is measured.

Various assay results are shown in Appendix A, where +++ indicates an EC₅₀ of <1 uM, ++ indicates an EC₅₀ of about 1-10 uM and + indicates an EC₅₀ of greater than 10 uM.

### Replicase Complex

Several compounds, including a compound from Appendix A, are assayed using a replicase complex. For preparing HCV replicase complex, HCV replicon cells (9-13) are cultured to confluence. Cells are lyzed with cold hypotonic buffer (10 mM Tris-HCl [pH 7.5], 10 mM NaCl) and are further broken with Dounce Homogenizer. Cellular debris and nuclei are pelleted by a brief spin (900×g at 4° C. for 5 minutes) and then the supernatant is transferred to a centrifuge tube. HCV replicase complex-enriched membrane fraction is collected with a one-time centrifugation of 15,000×g at 4° C. for 20 minutes. The resulting pellet is re-suspended with storage beffer (hypotonic buffer containing 15% glycerol) at a ratio of 1 mL for 1×10⁸ cells.

In an in vitro replicase complex assay 6 ul of membrane fraction is mixed with 44 ul reaction mixture (68 mM HEPES [pH 7.3], 13.6 mM KCl, 13.6 mM MgCl₂, 400 uM MnCl₂, 60 unites RNase inhibitor, 13.6 ug/mL actinomycin D, 10 ul of NTP mixture (3 mM ATP, GTP, UTP, and 10 uCi α[P³²] CTP with a specific activity of 800 Ci/mmol) and the test compound. RNA synthesis reaction mixture is incubated at 30° C. for 1 hour and stopped by TRIzol extraction followed by isopropanol precipitation. RNA is separated on a 1% agarose gel. The gel is dried prior to autoradiography.

An in vivo replicase complex assay is also performed in which HCV replicon cells are treated with compound before the preparation of replicase complex and RNA synthesis.

Based on either in vitro or in vivo replicase complex assay, EC₅₀ is calculated, and, where available, results parallel those obtained when using HCV replicon.

### Viral Protein Assay

A compound from Appendix A is also tested using HCV polyprotein. Viral protein levels after treatment with the test compound or other classes of inhibitors is determined by immunoblotting. In the experiment, 5×10⁶ cells/dish of cells expressing the HCV replicon are seeded onto 150 mm dishes. 48 hours later when cells grew to more than 80% confluency, test compounds are added and the cells are incubated for 2 to 24 hours. Cells are collected and lysed in 1.5 ml 1×NPB buffer (50 mM Tris-pH 7.5, 150 mM NaCl, 1% Sodium Deoxycholate, 0.1% SDS) plus protease inhibitors (Complete, Mini, EDTA-free. Roche, Cat # 1836170). Cells are centrifuged at 14000 rpm (20817×g) at 4° C. for 15 minutes or pass the lysate through QIAshredder (Qiagen, Cat 79654). The supernatant of cell lysate is transferred to a fresh tube and used for immunoprecipitation or stored at −80° C.

For detection, anti-NS3 (Anogen, Cat. MO-140018K) or anti-NS4A (Biodesign, Cat. C8A236M) is pre-bound to protein A beads at 4° C. for 1 hour. The bound antibody is then added to cell lysate and incubated at 4° C. overnight rocking gently. The beads are washed four times with PBS plus 0.05% Tween 20 and protease inhibitors. The proteins are resolved on 4 to 20% or 10 to 20% gradient Tris HCl gel. The sample in the gel is then transferred to membrane (Immun-Blot Immun-Blot PVDF/Filter Paper Sandwich) according to manufacturer's manual.

The membrane is blocked in 5% dry milk in PBST (PBS plus 0.05% Tween 20) at room temperature for 1 to 2 hours. The blocking solution is removed and the first antibody solution (anti-NS4A, anti-NS3, anti-NS5A, anti-NS4B in 2.5% dry milk in PBST) is added. The membrane is rocked gently at room temperature for 2 hours. The membrane is washed three times for 15 minutes/wash in PBST. Secondary antibody in 2.5% dry milk in PBST is then added and incubated at room temperature for 1 hour. The membrane is washed twice with PBST and twice with PBS. The membrane is developed with ECL plus (Amersham Biosciences, Cat # RPN2133) and exposed to X-ray film. EC₅₀ is calculated for the tested compound and results for this compound parallel those in Appendix A.

All publications and patents mentioned in the above specification are herein incorporated by reference. The above description, drawings and examples are only illustrative of preferred embodiments that achieve the objects, features and advantages of the present invention. It is not intended that the present invention be limited to the illustrative embodiments. Any modification of the present invention which comes within the spirit and scope of the following claims should be considered part of the present invention.

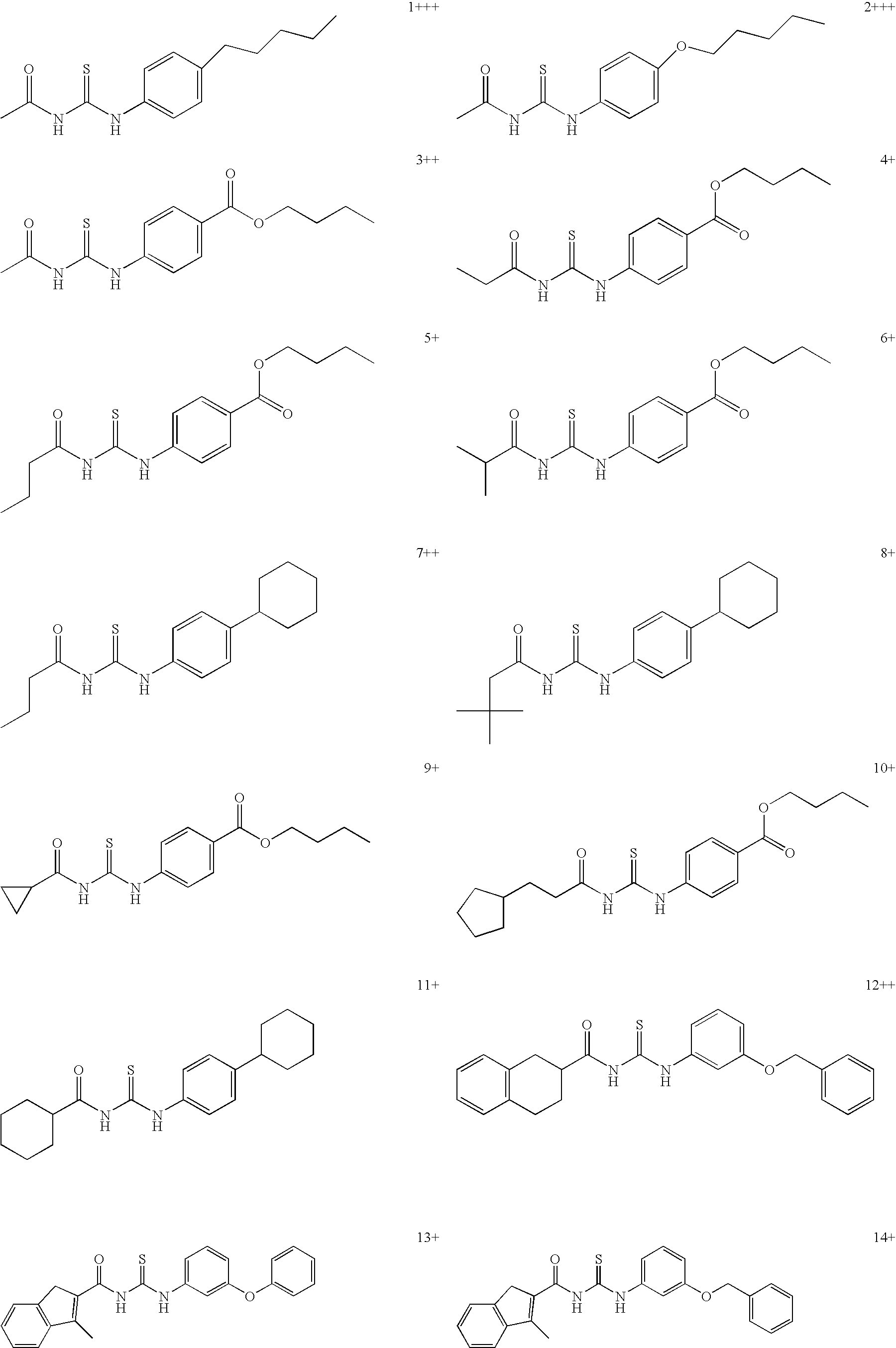

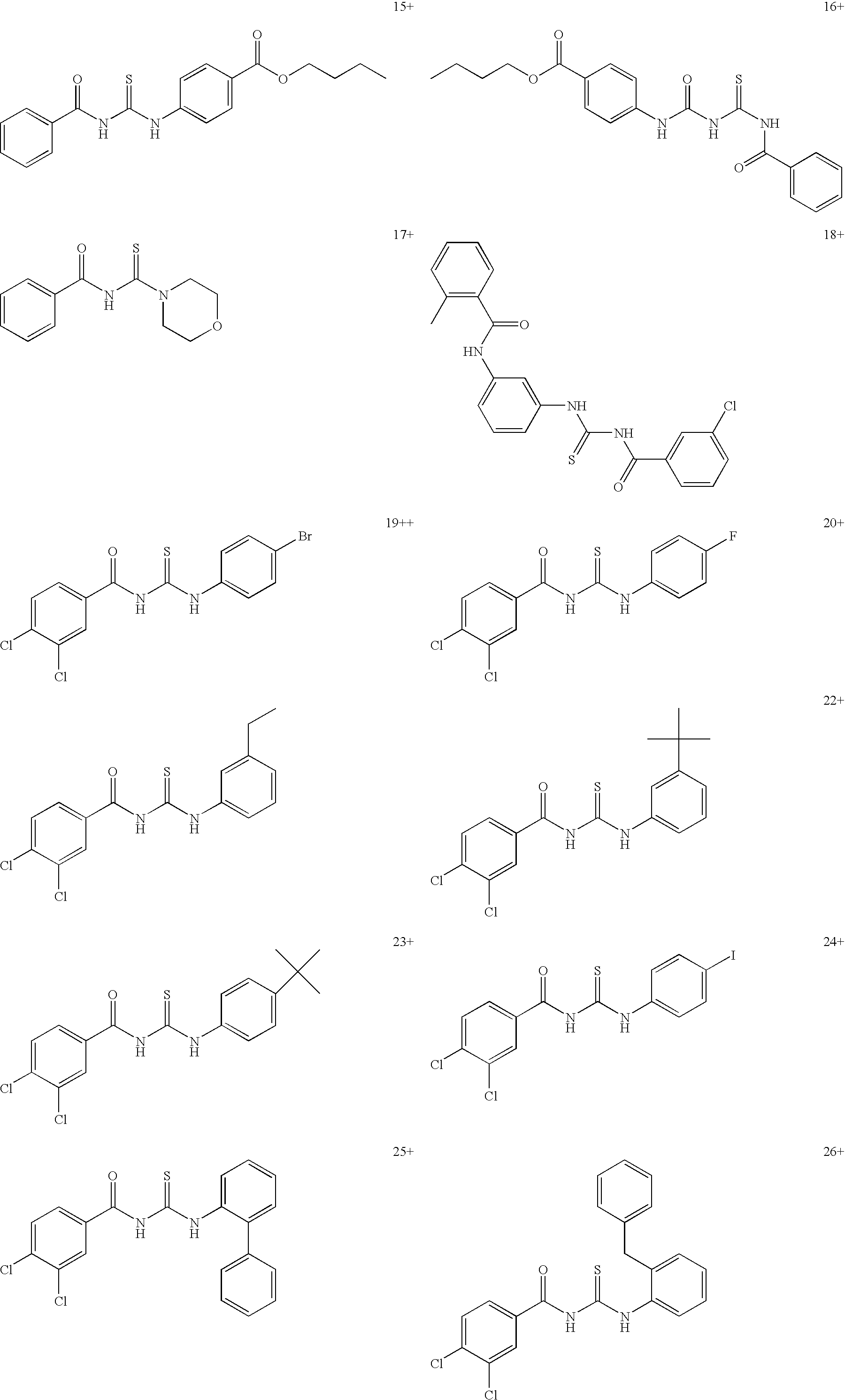

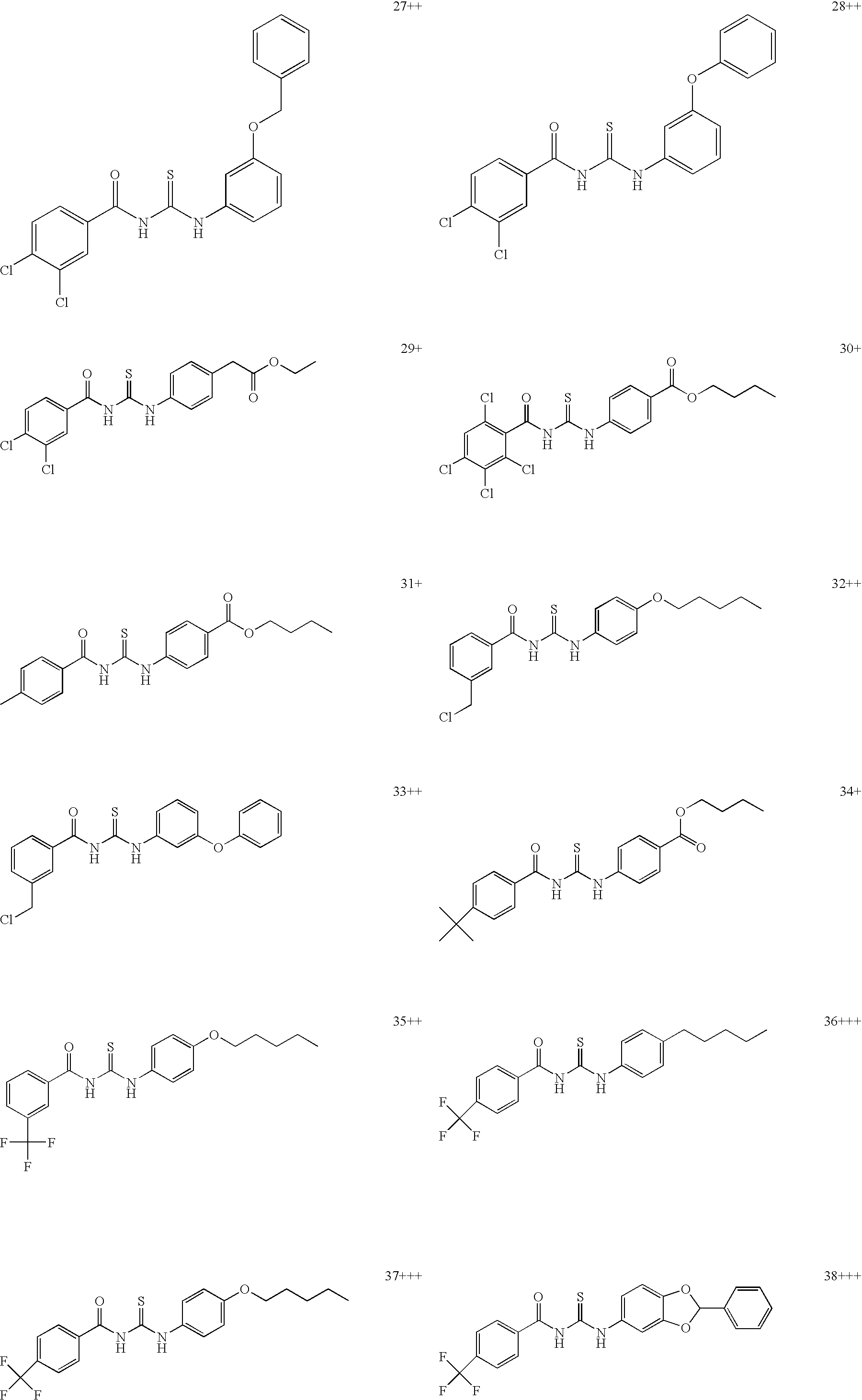

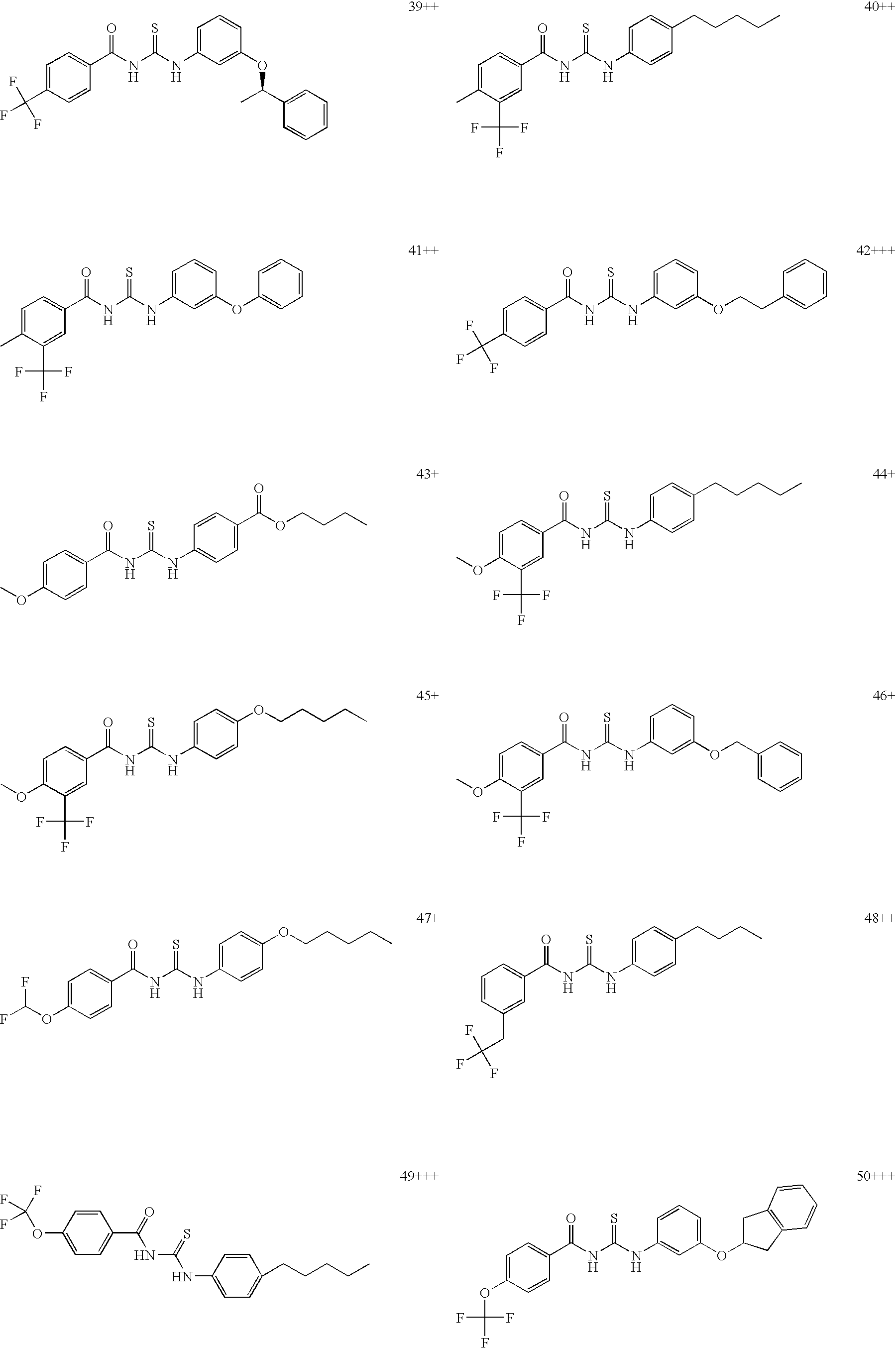

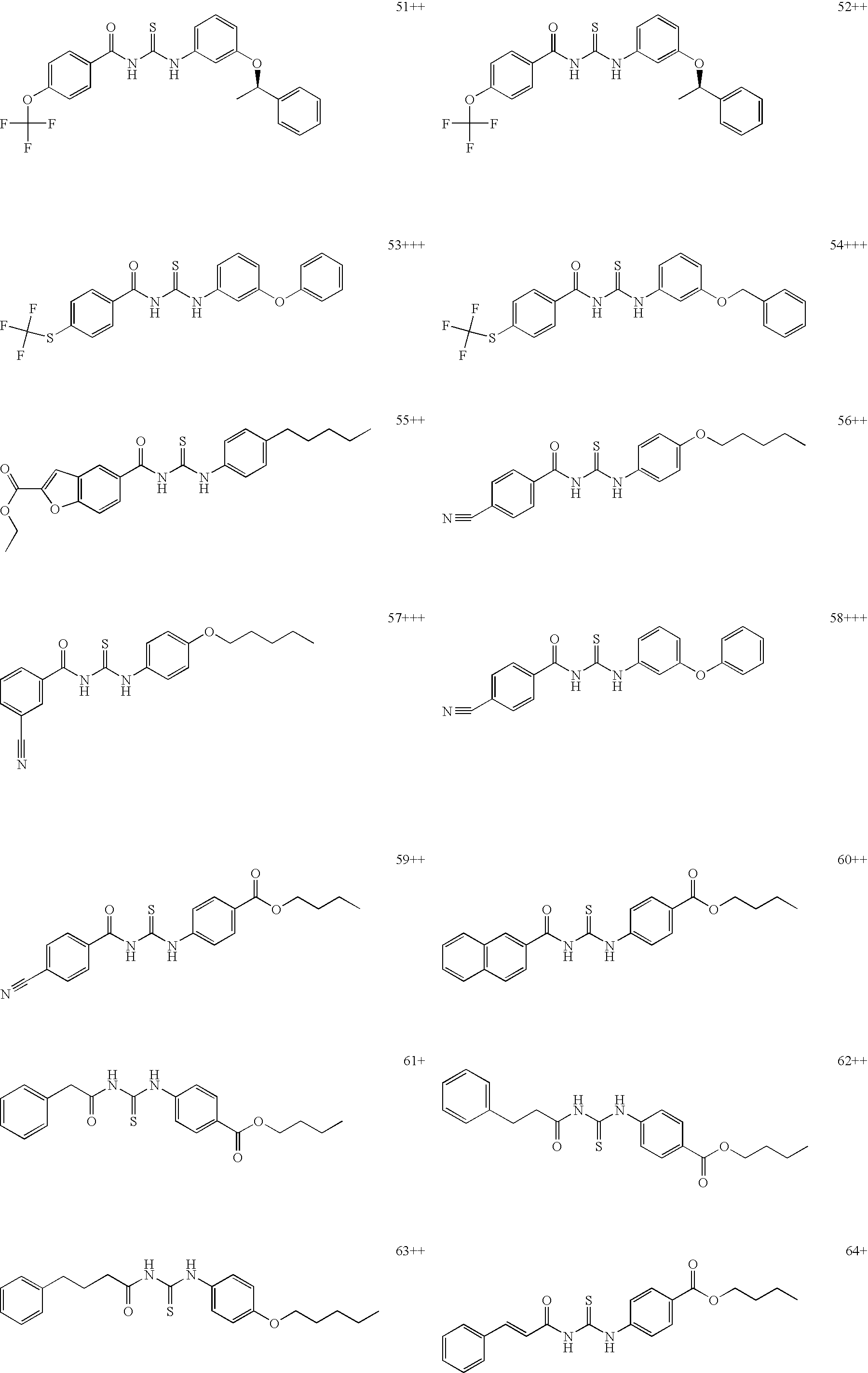

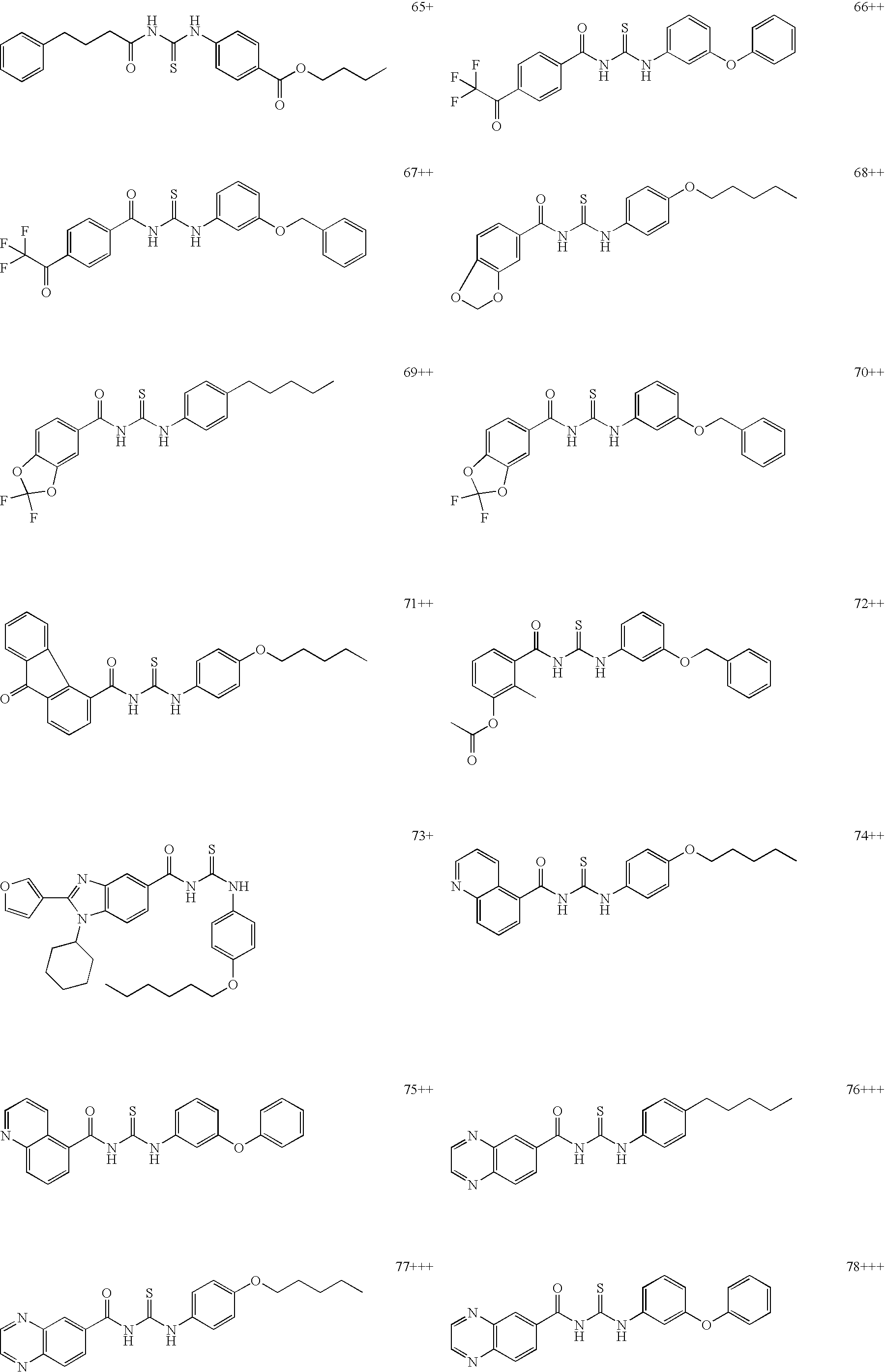

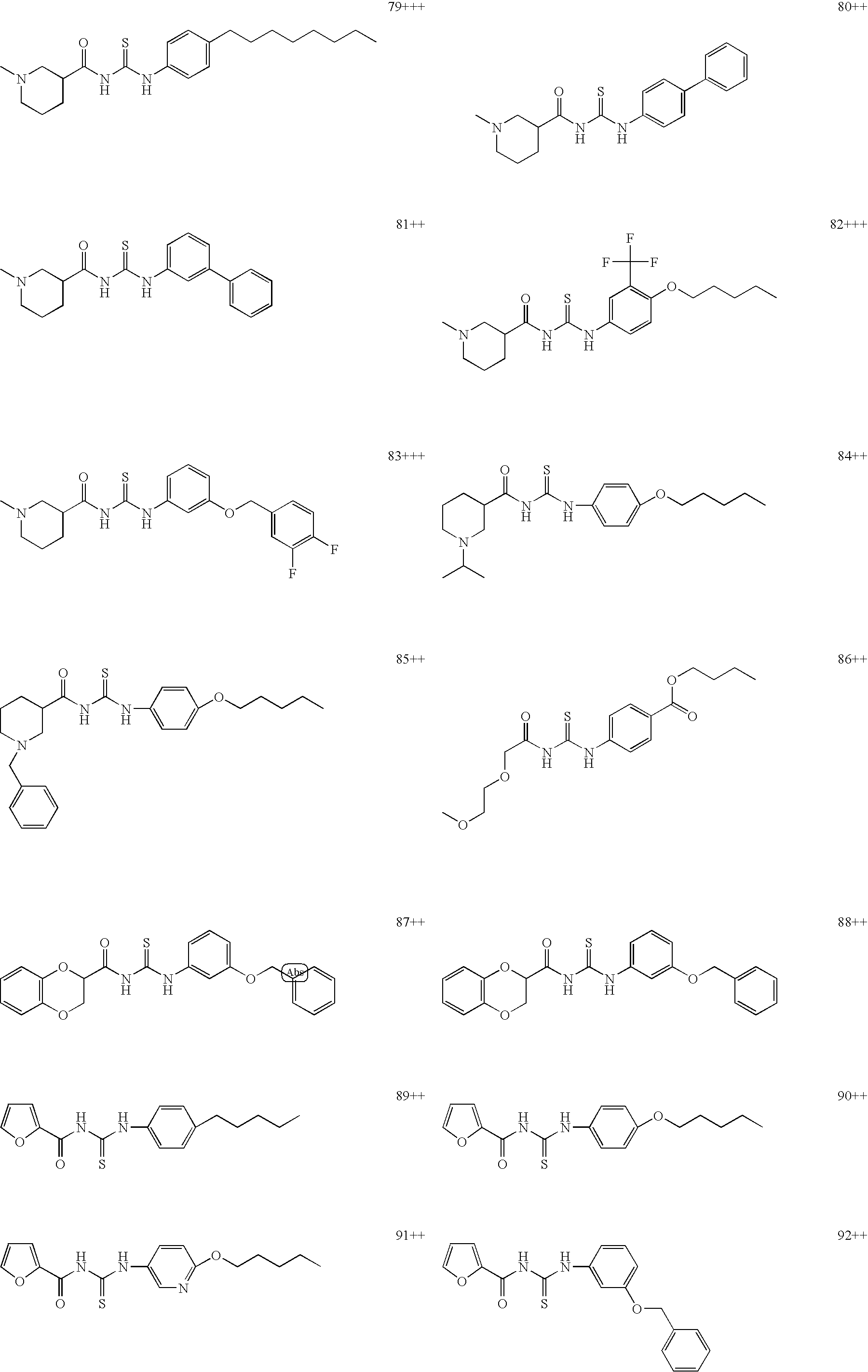

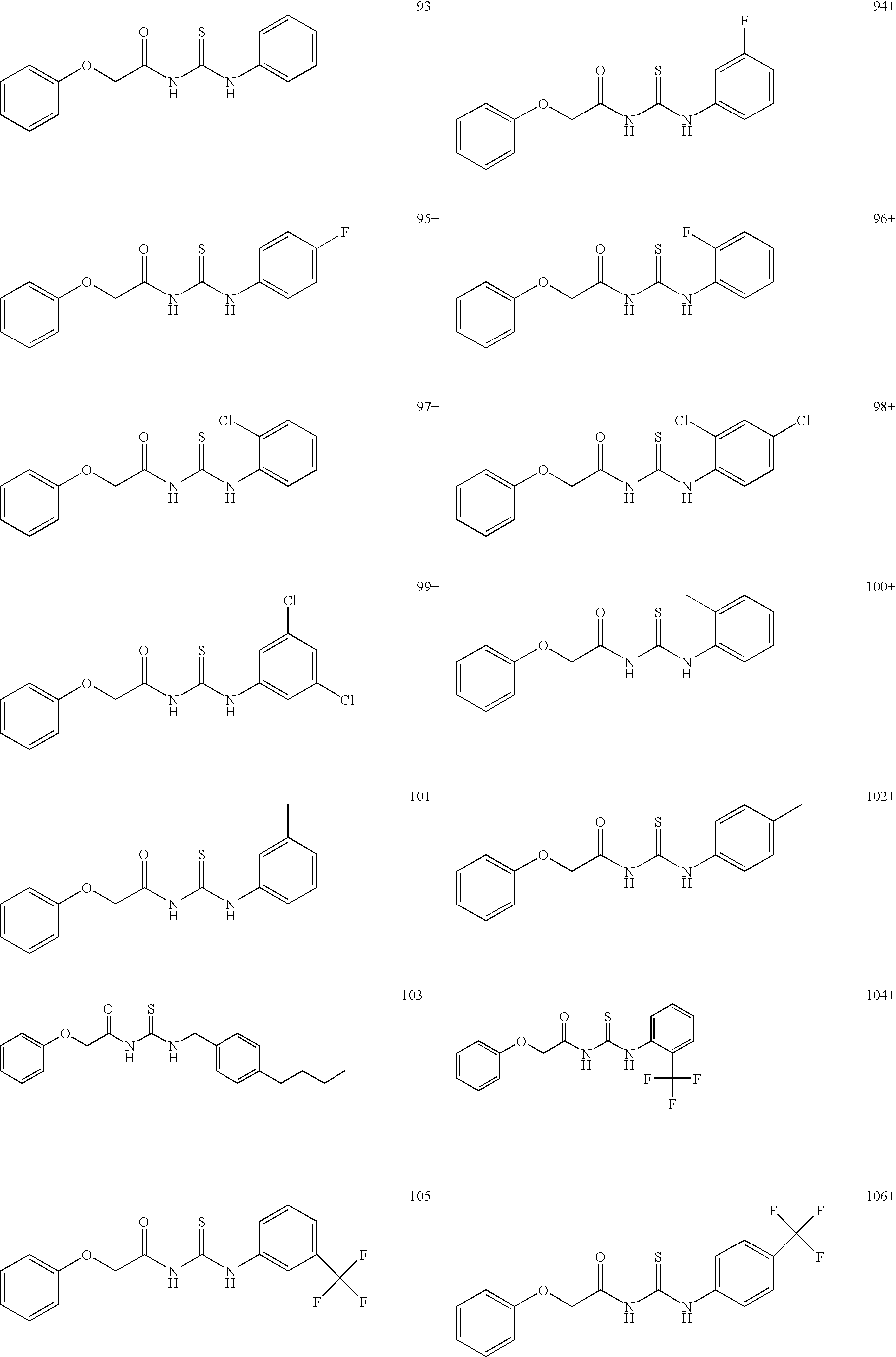

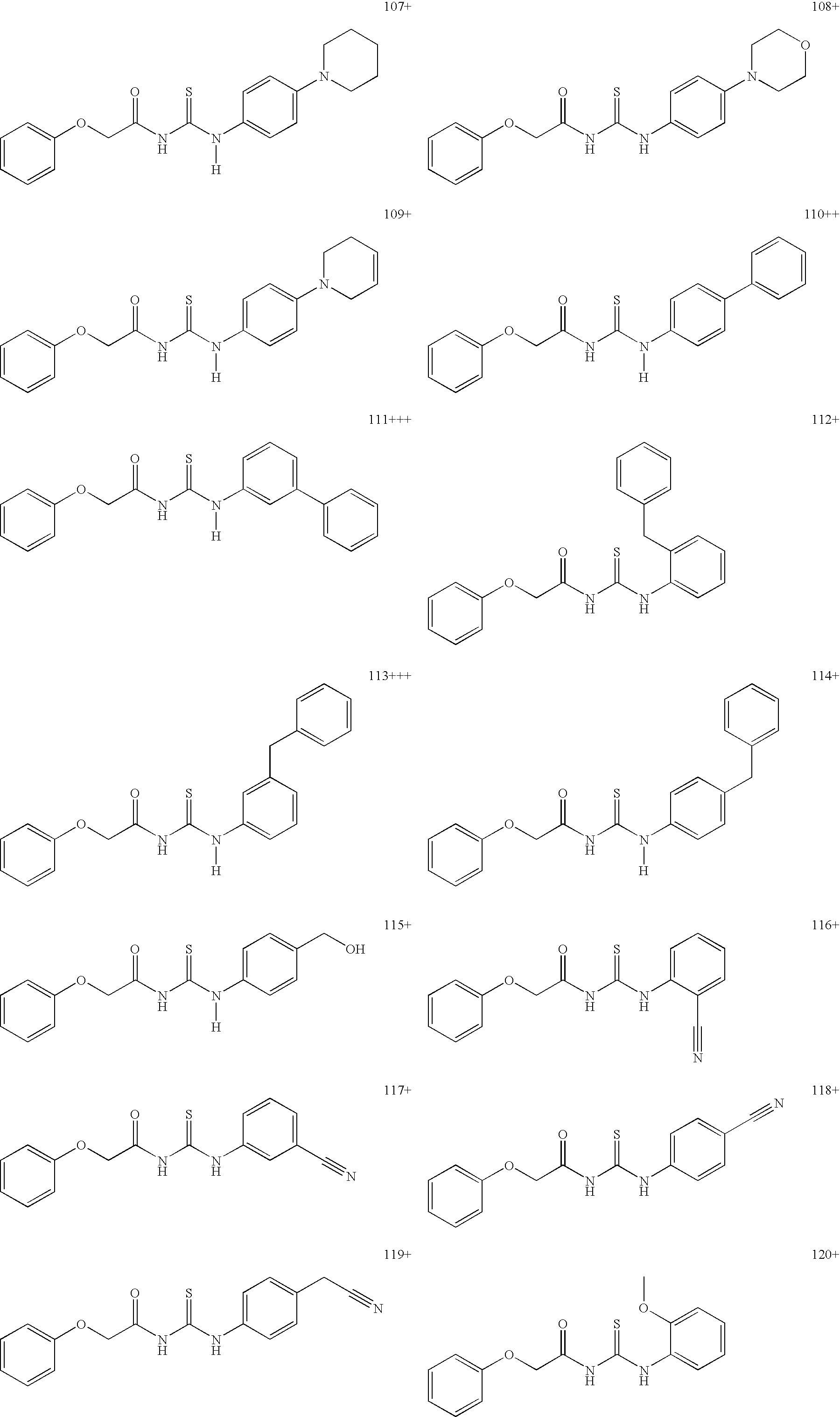

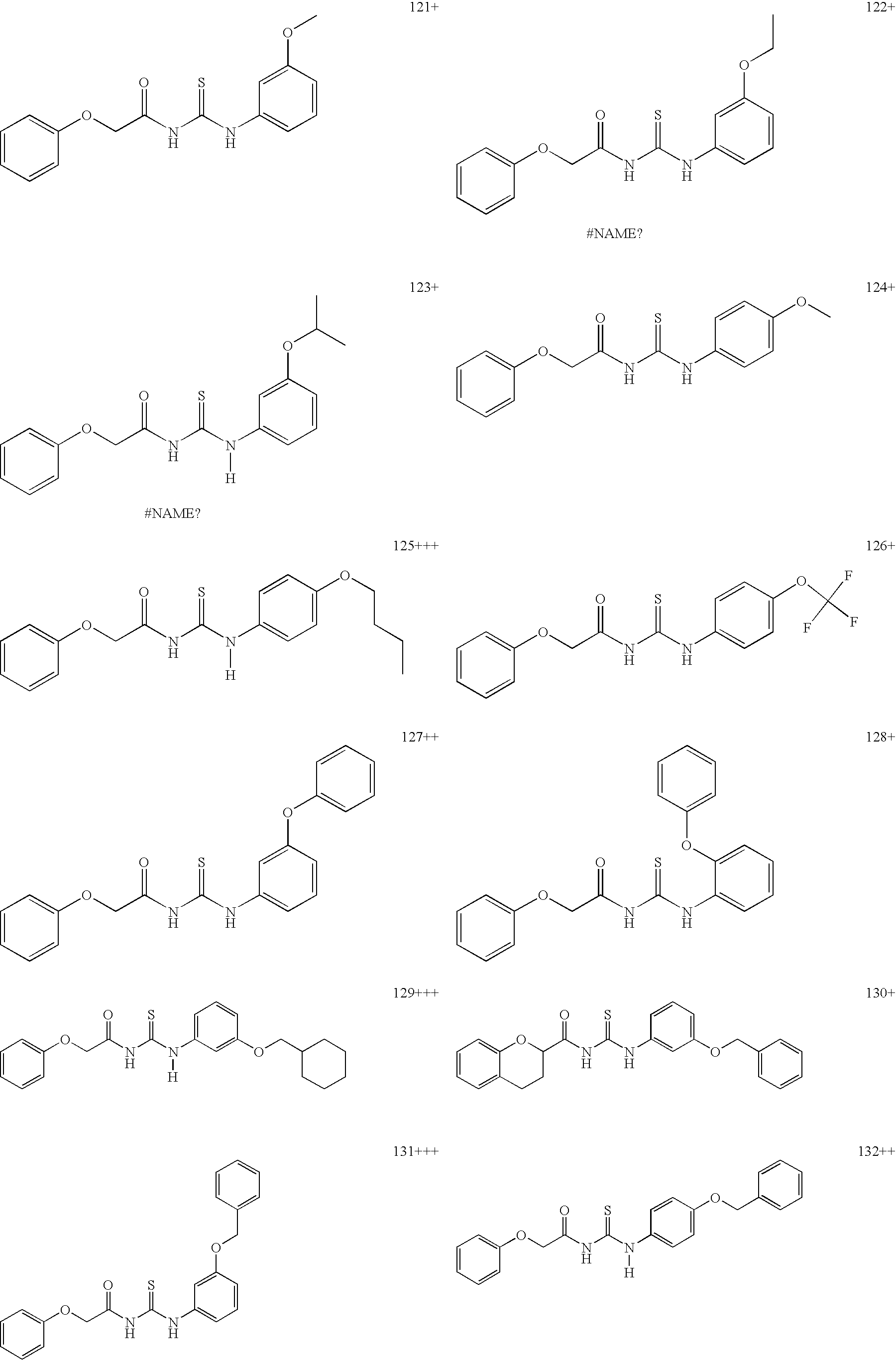

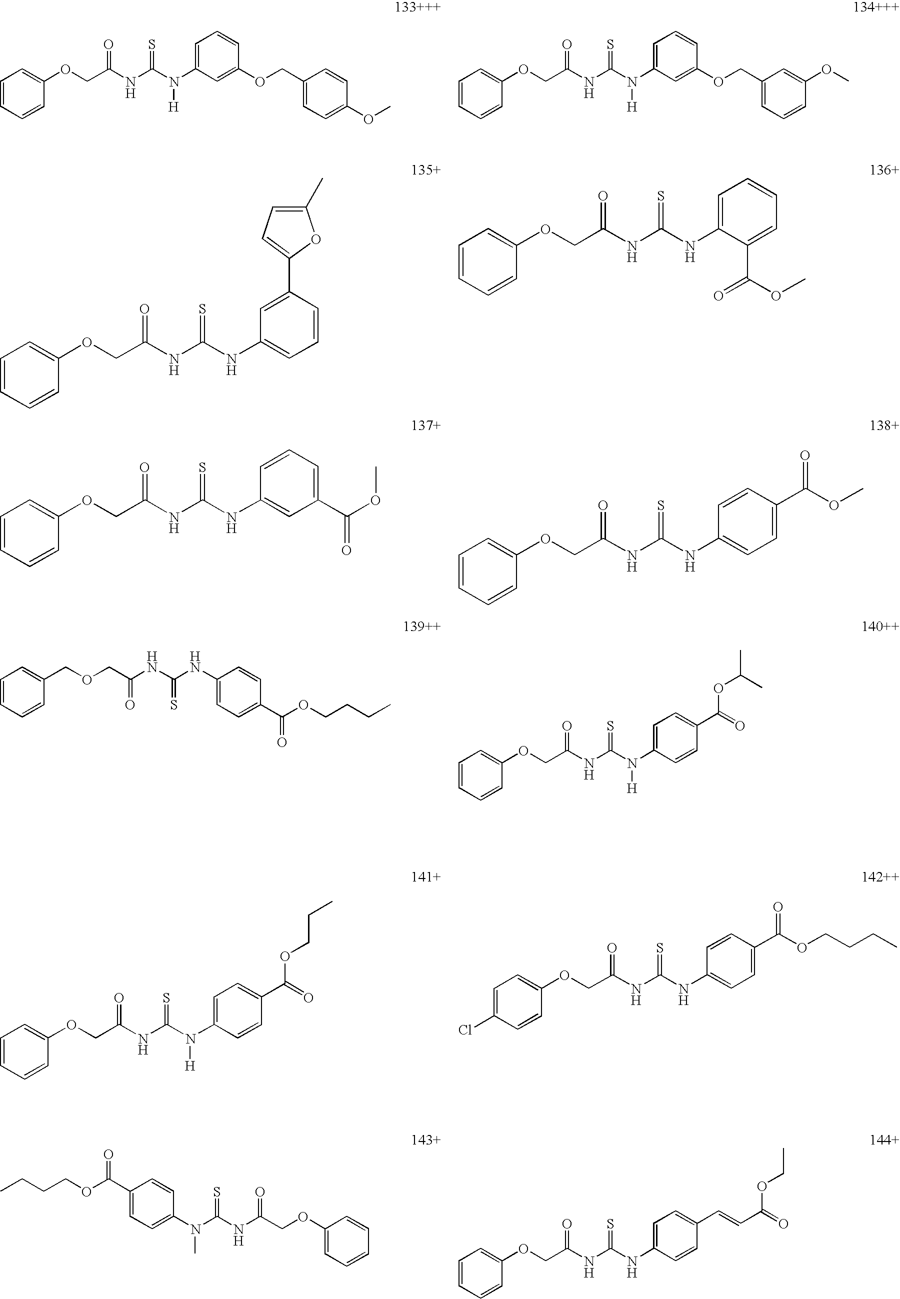

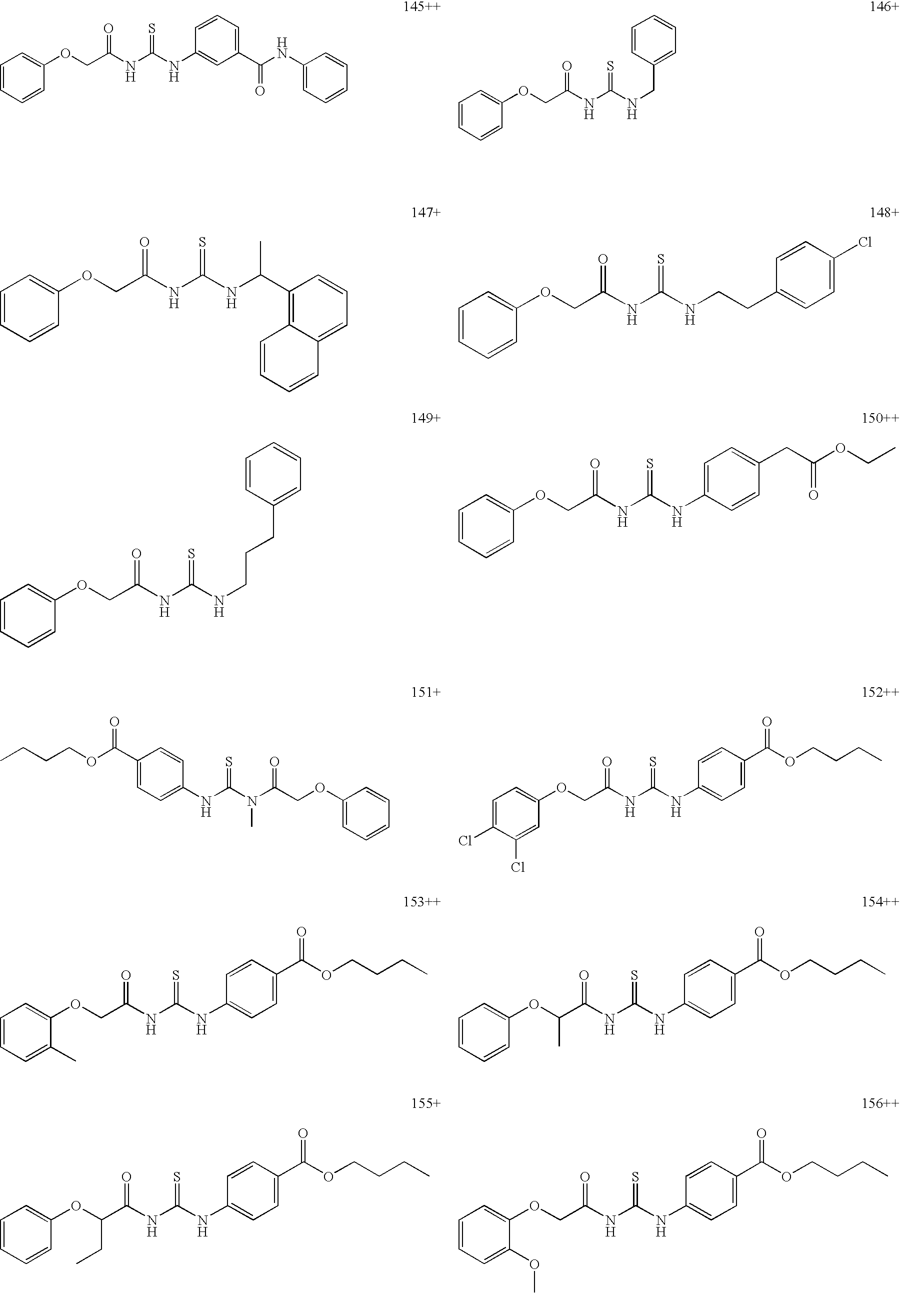

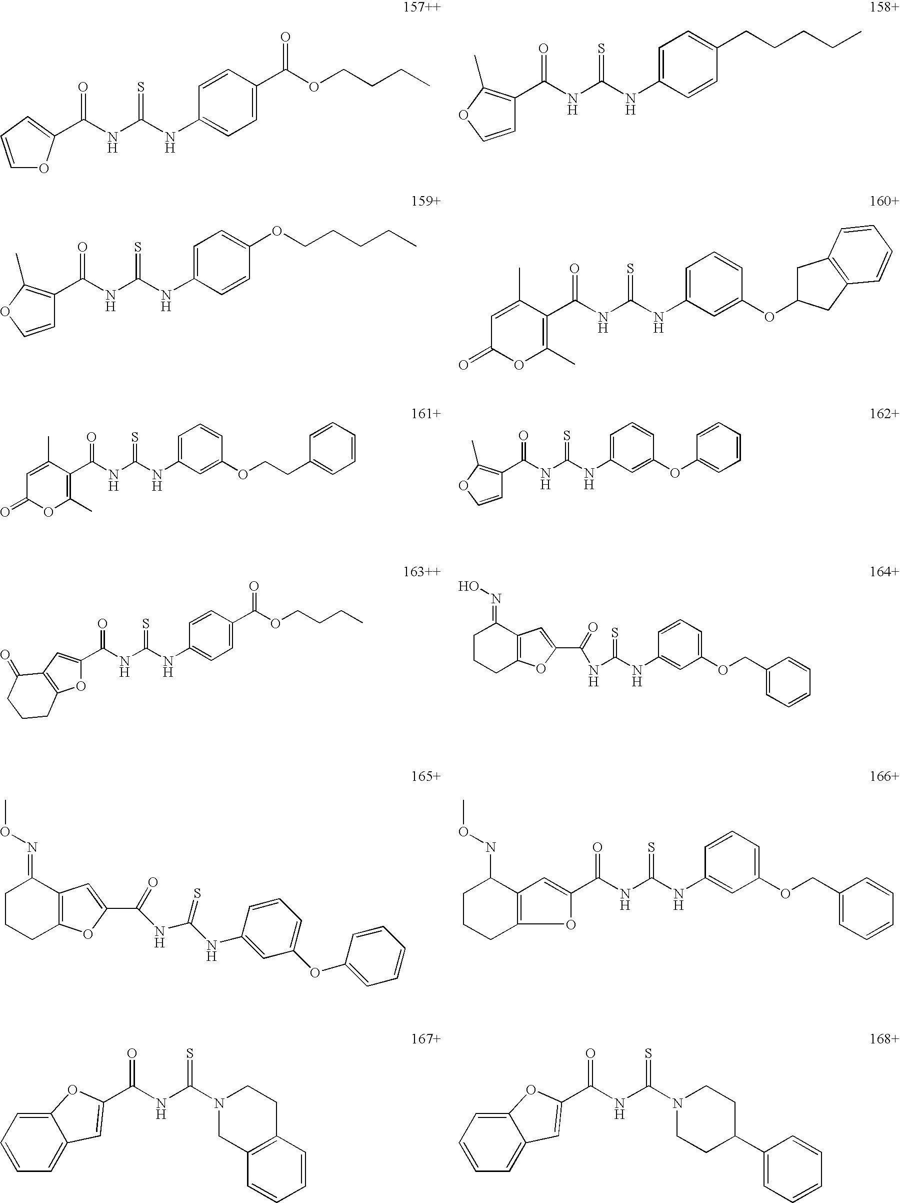

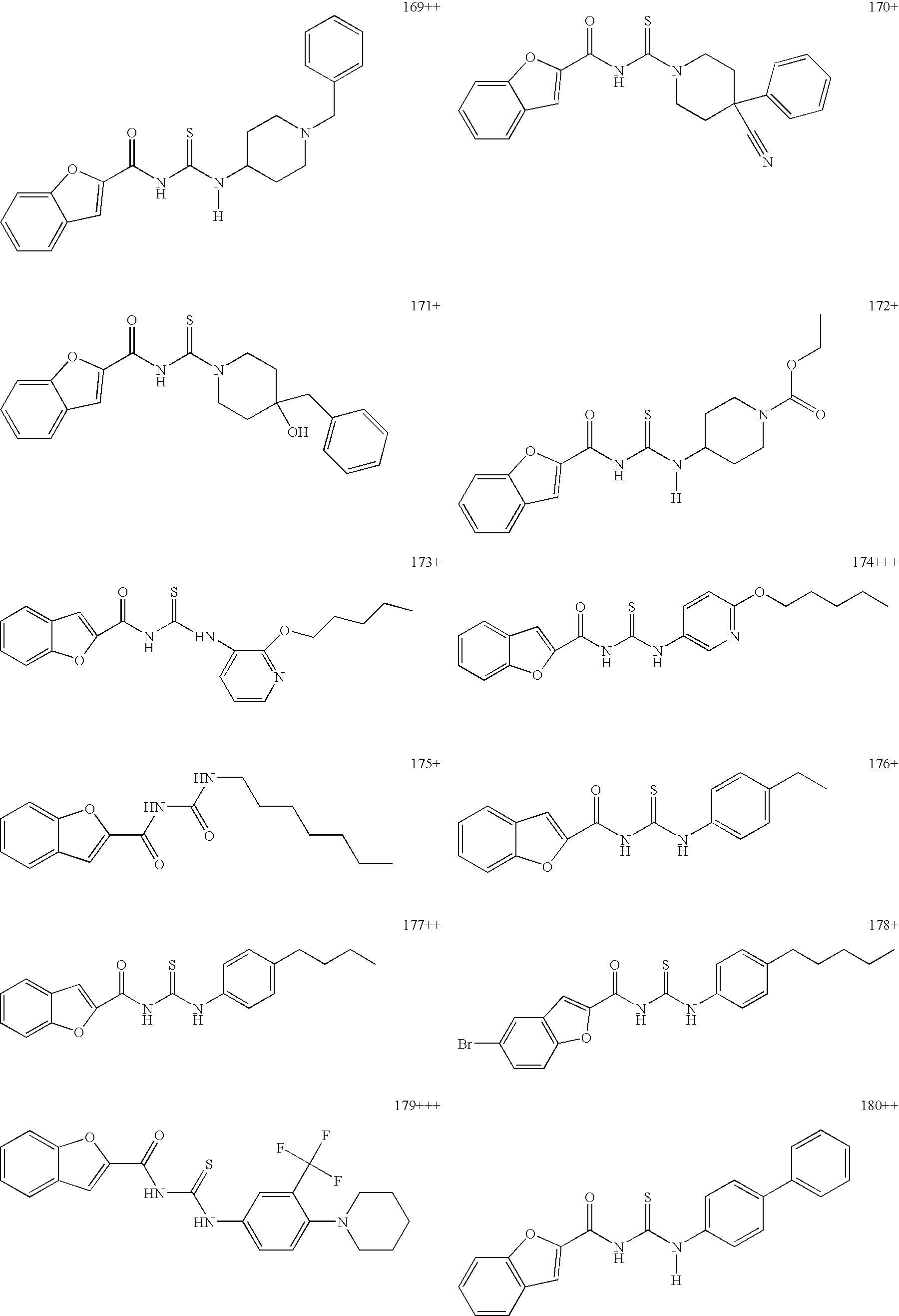

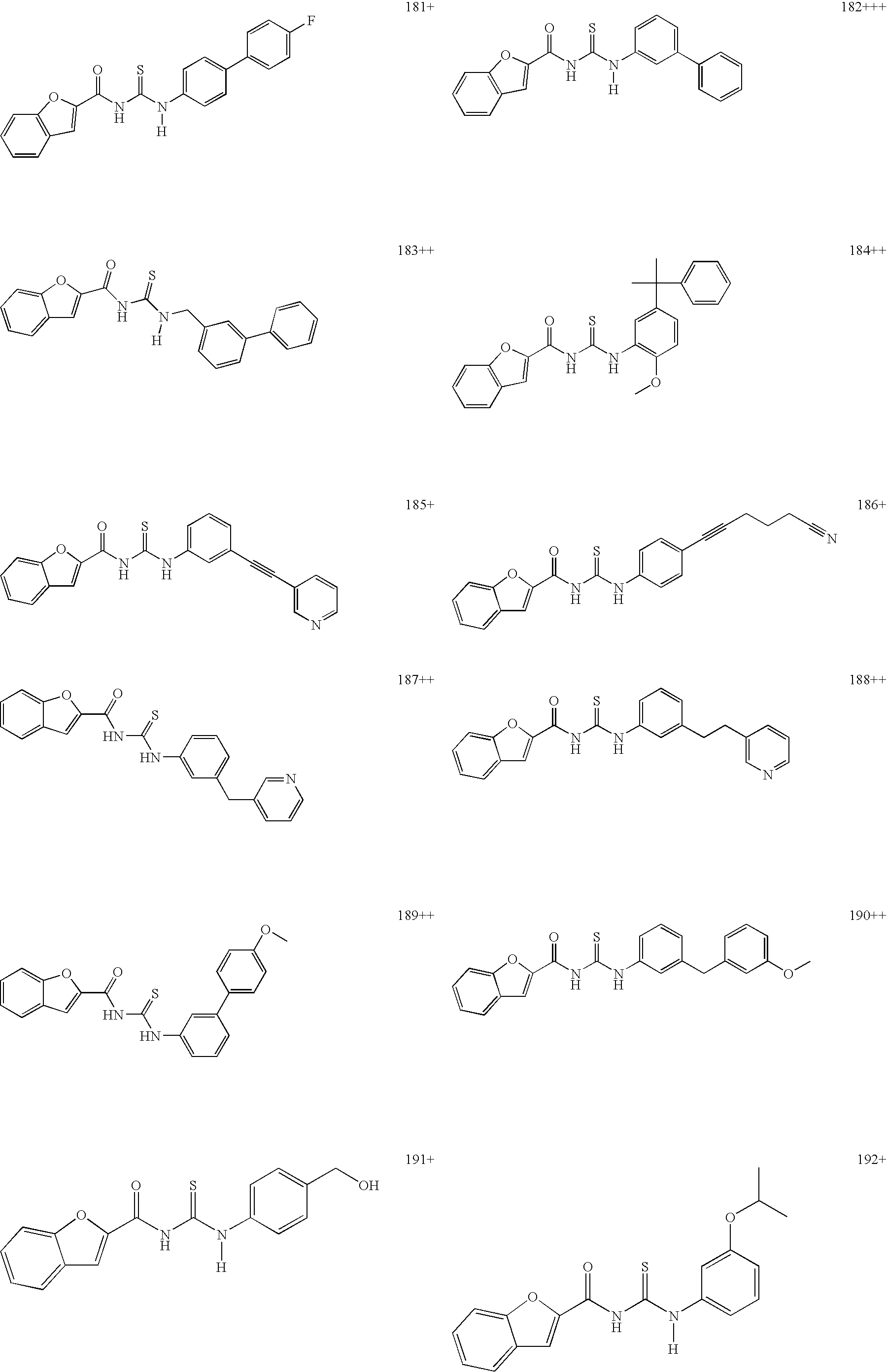

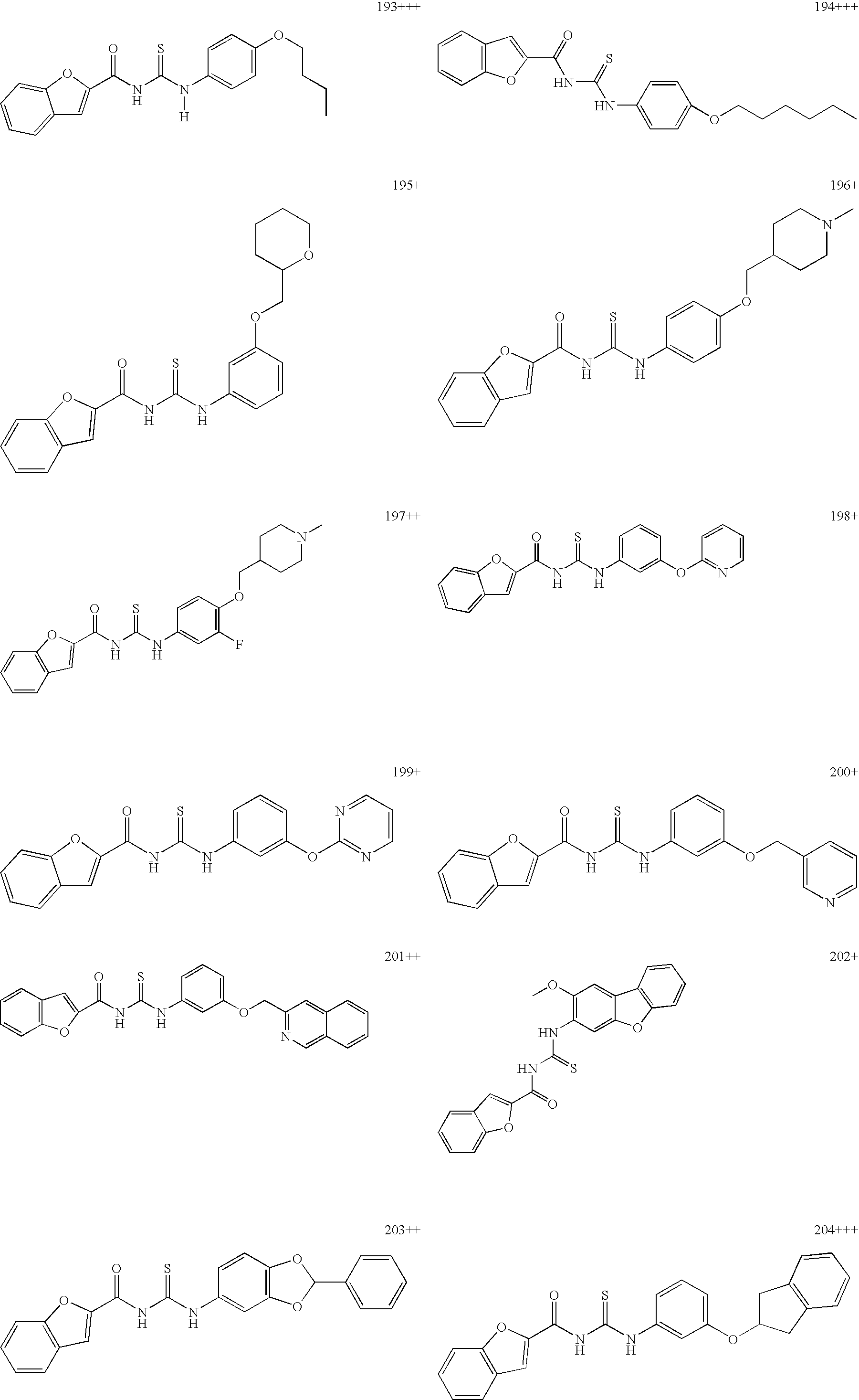

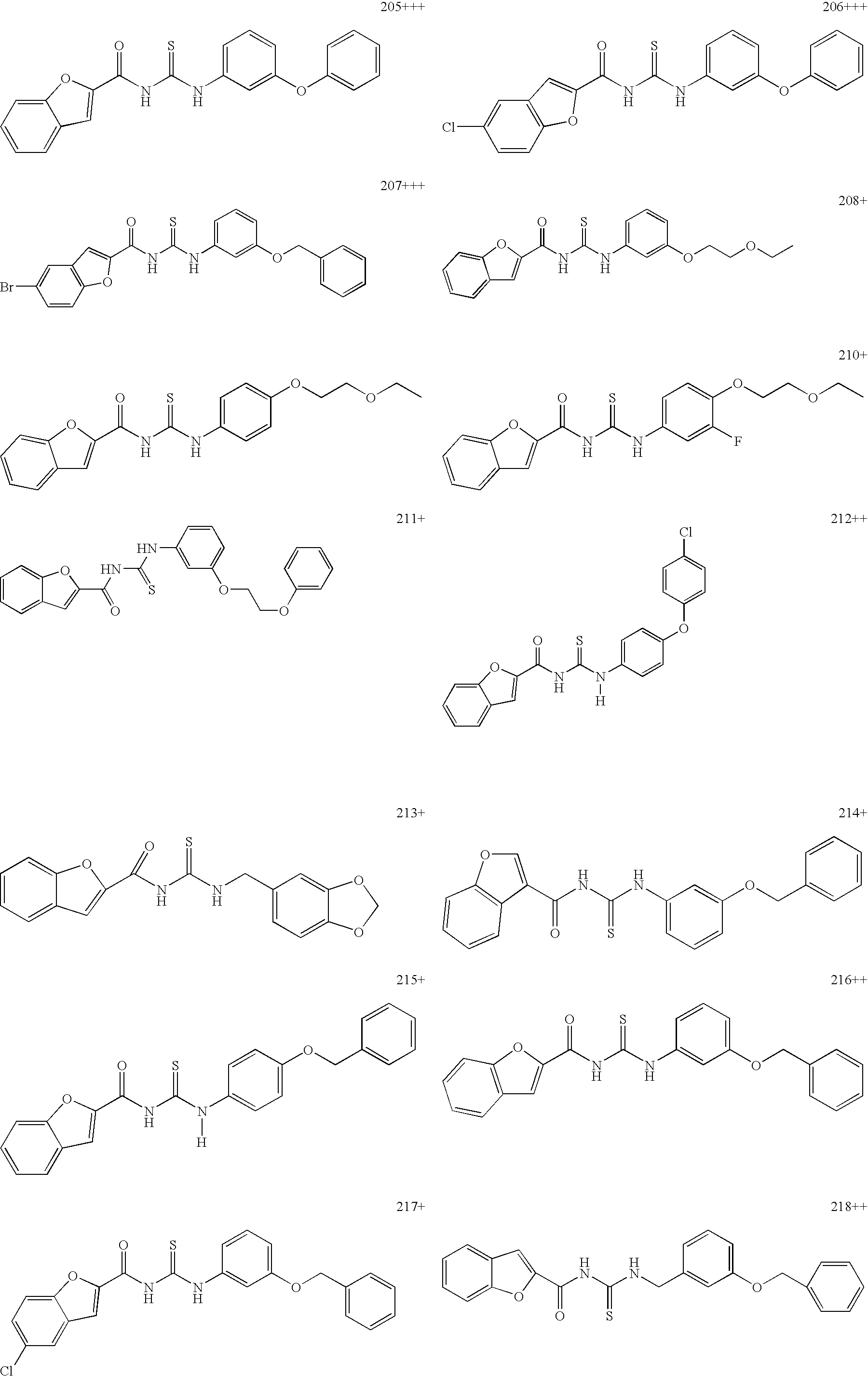

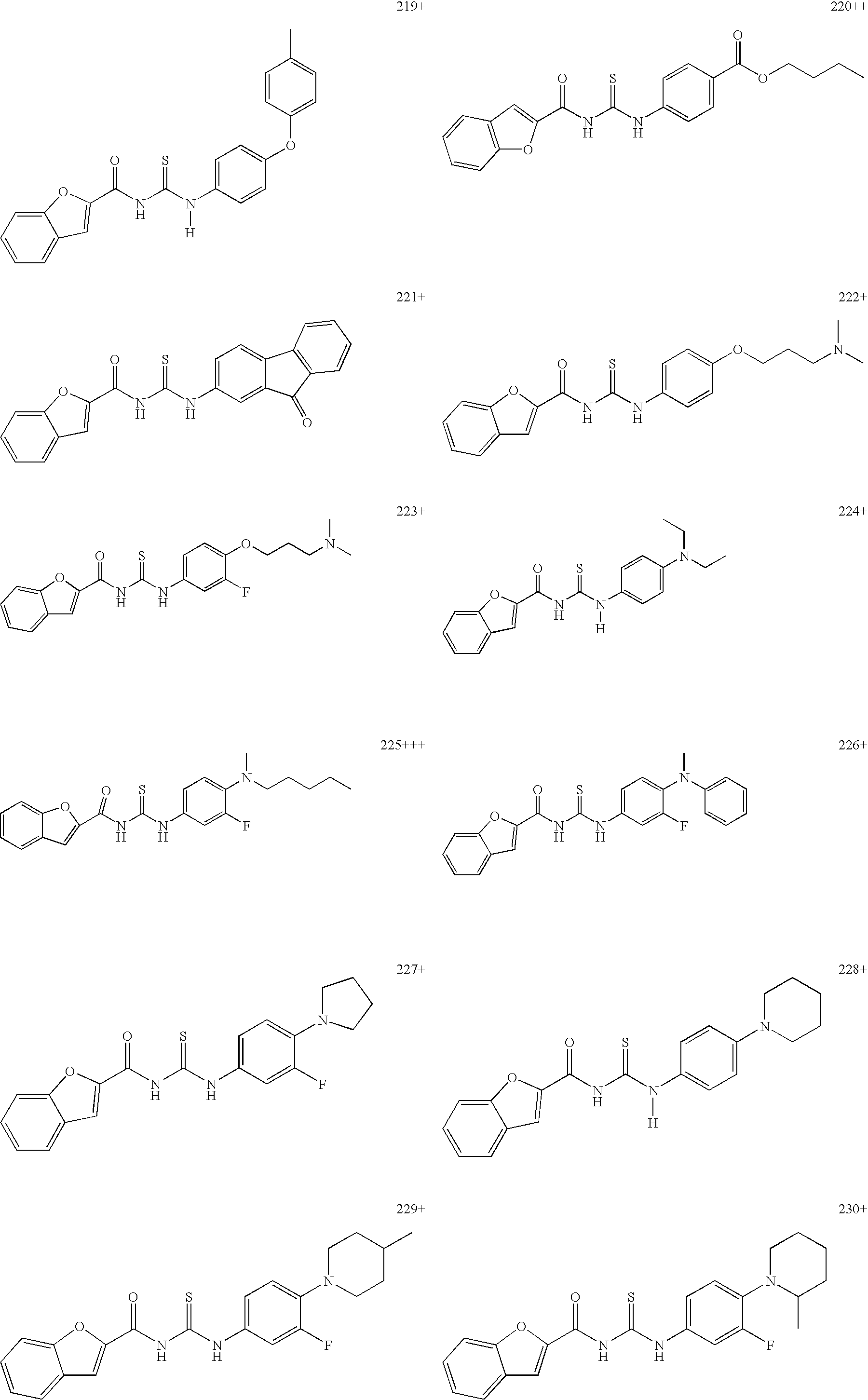

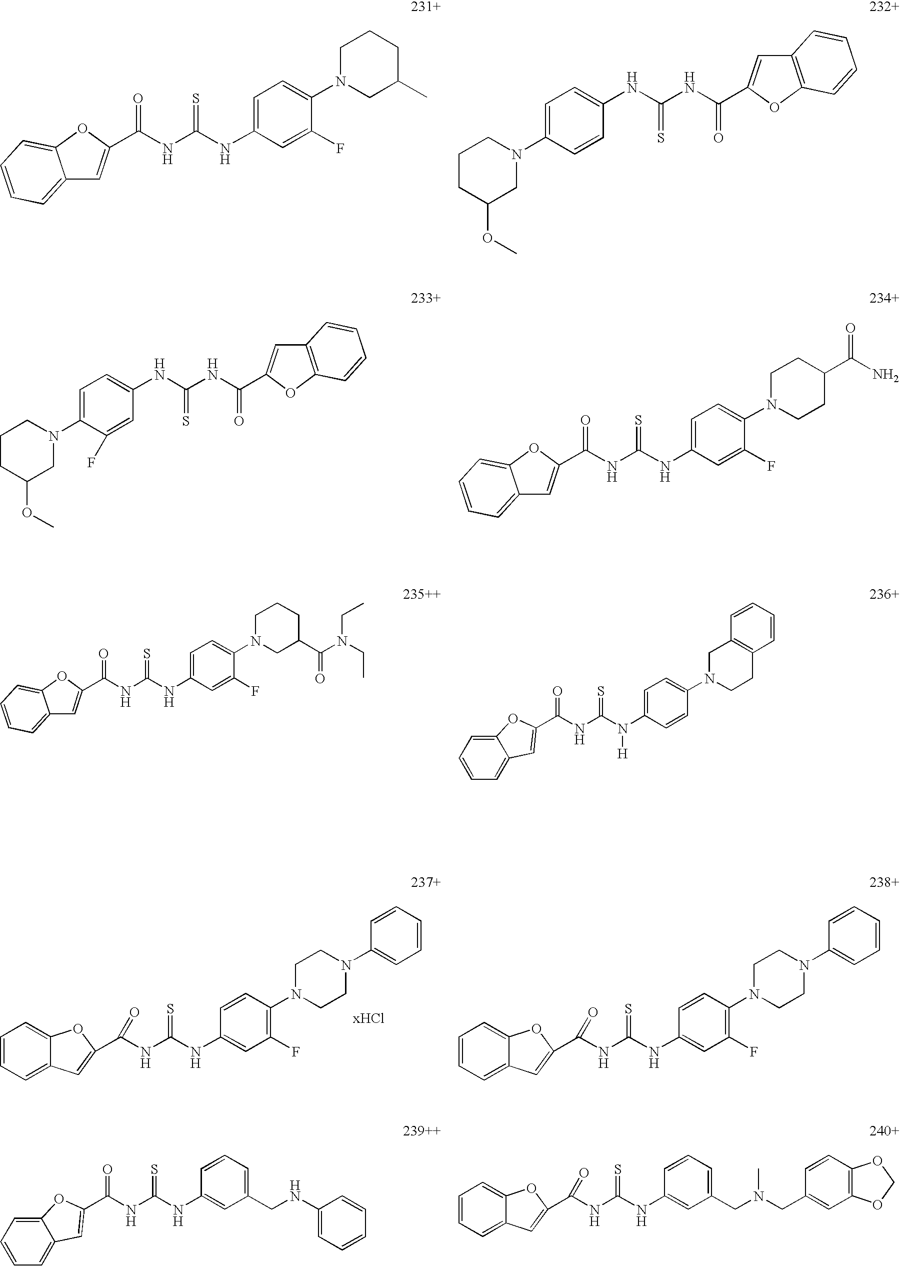

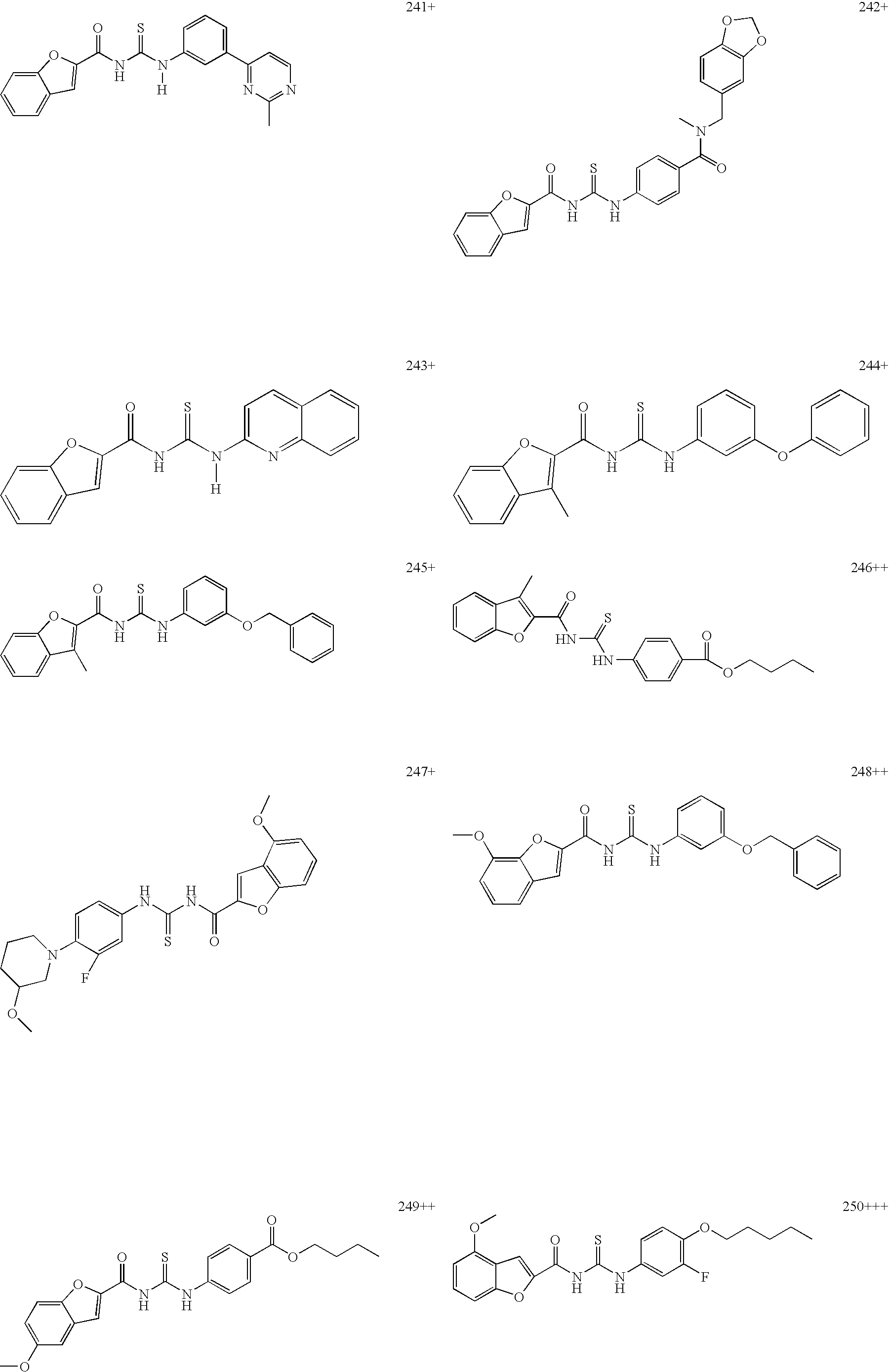

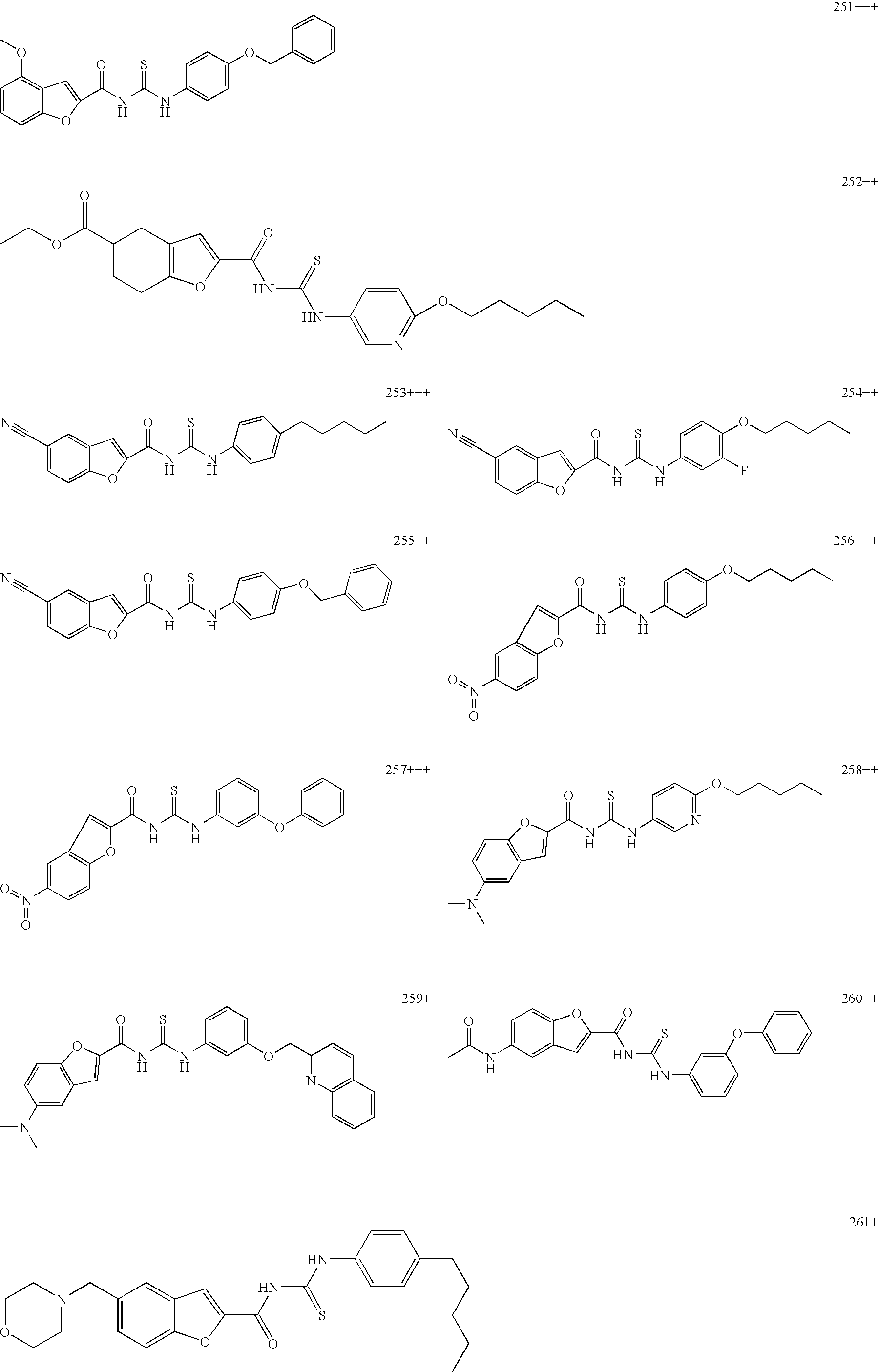

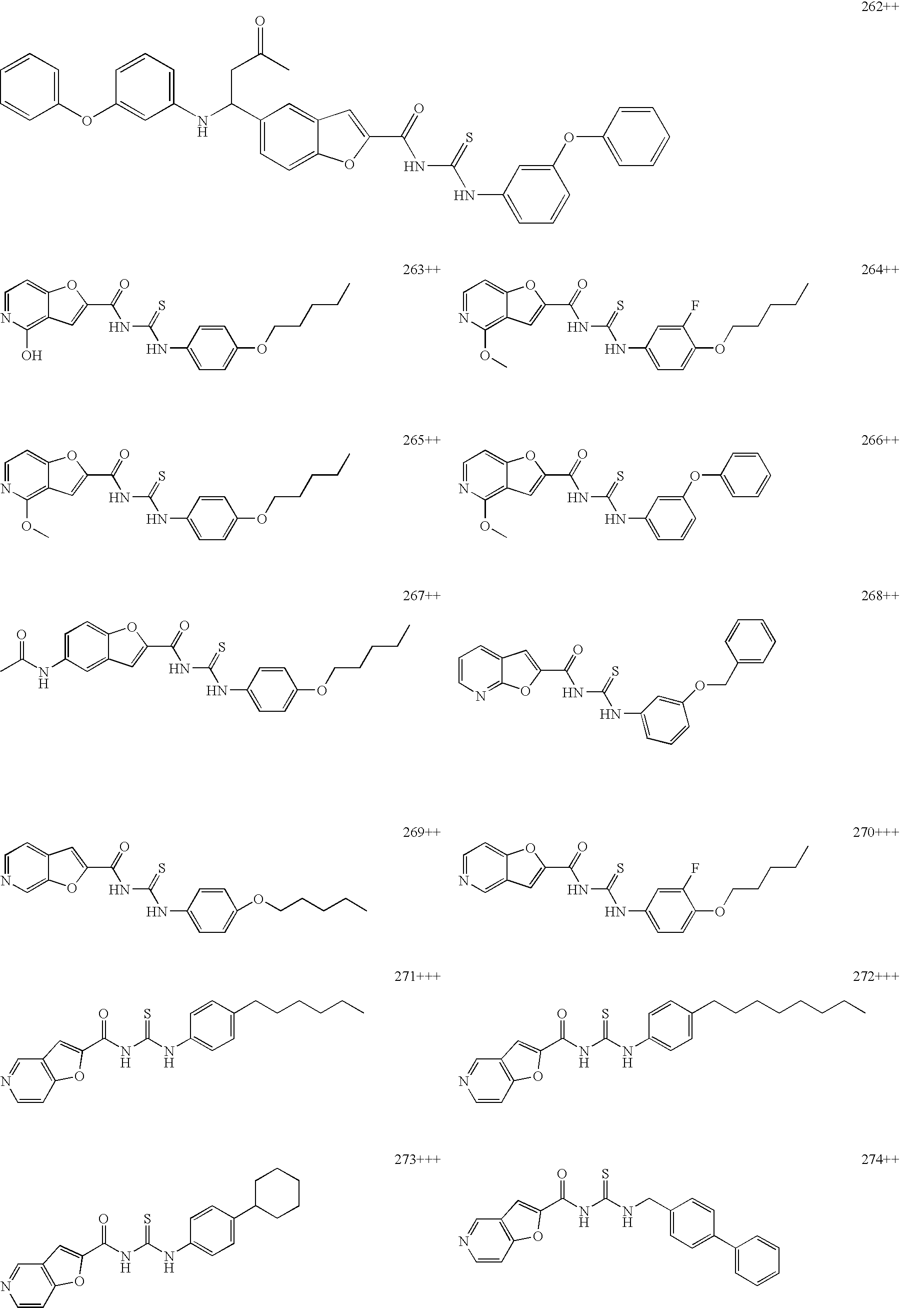

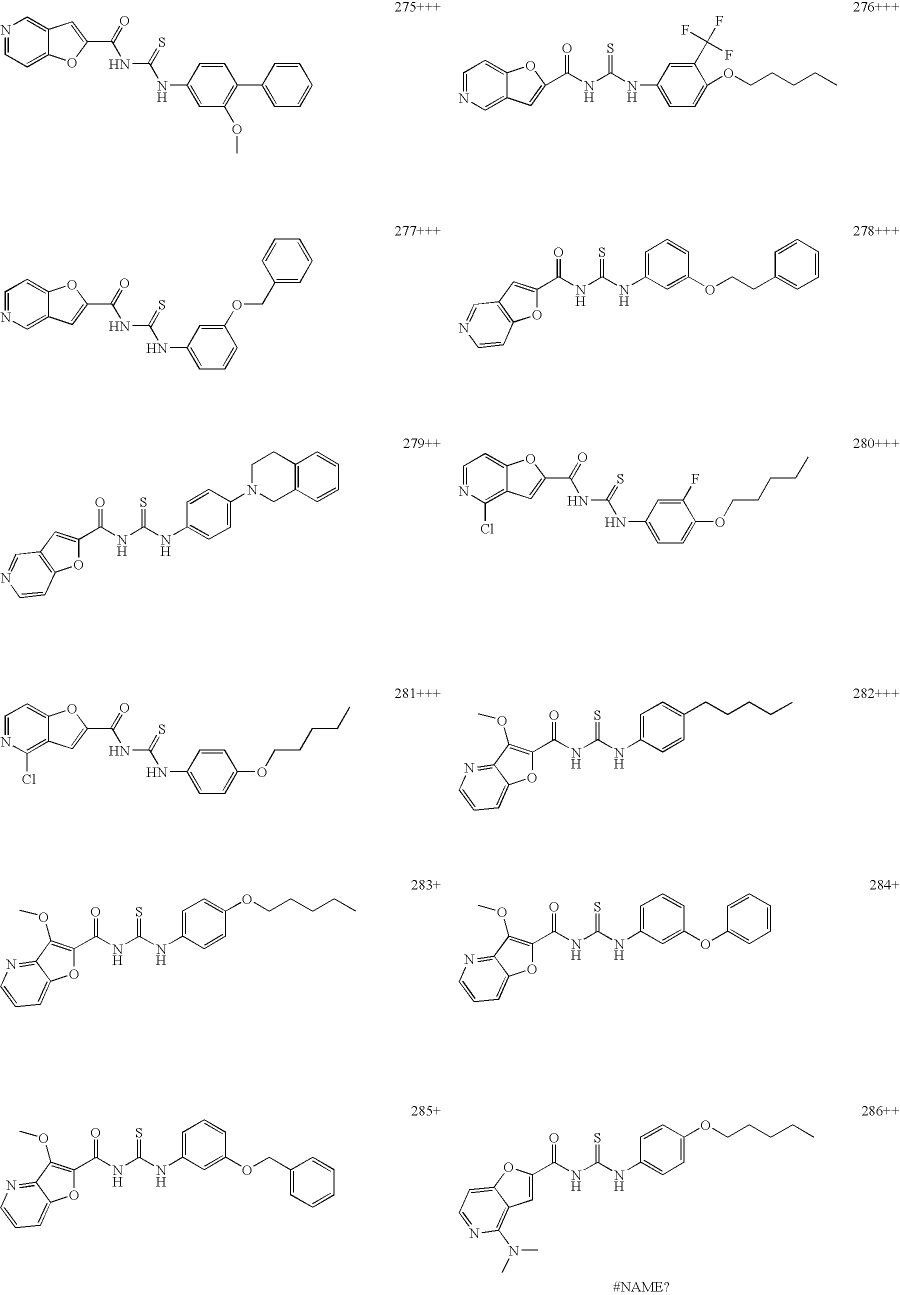

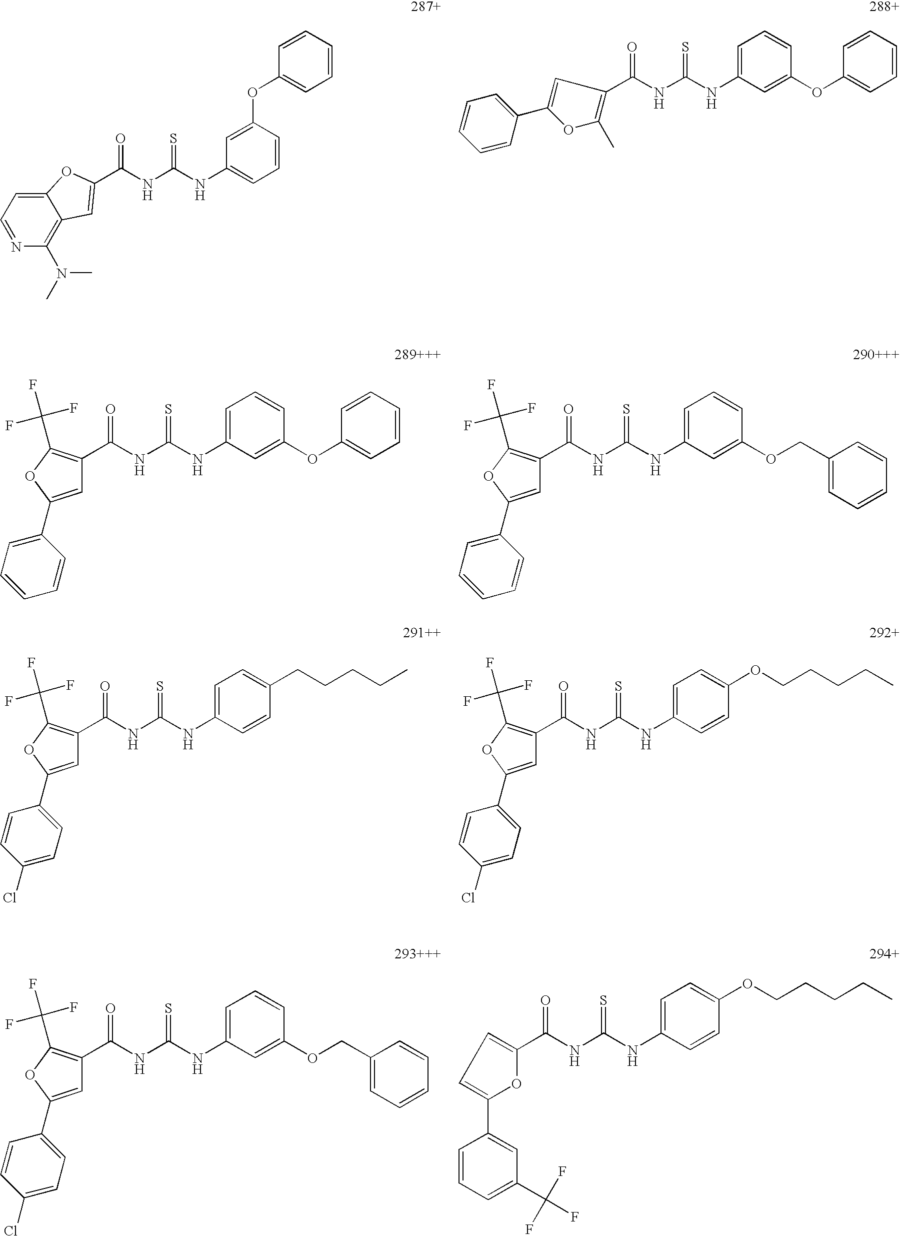

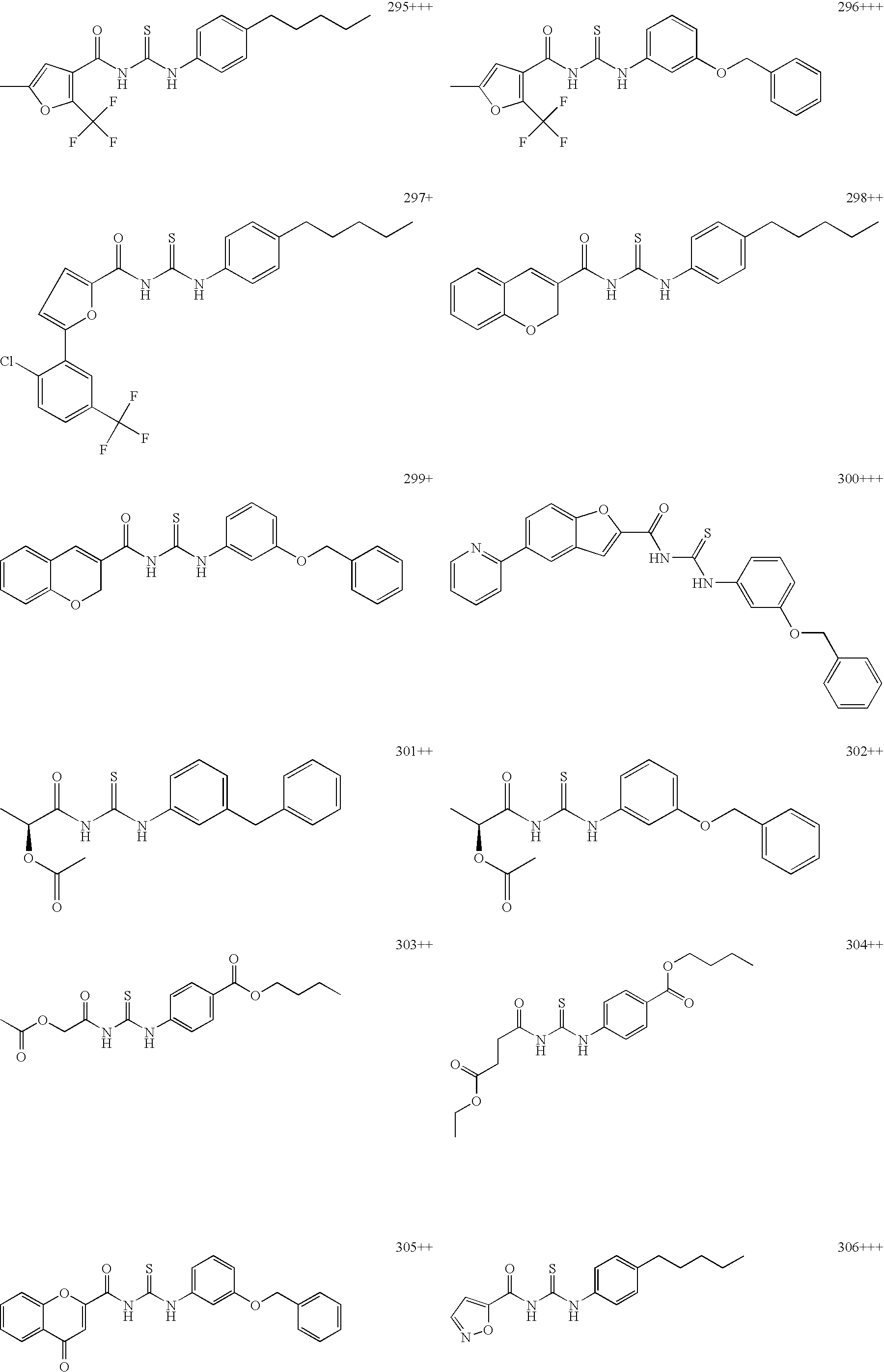

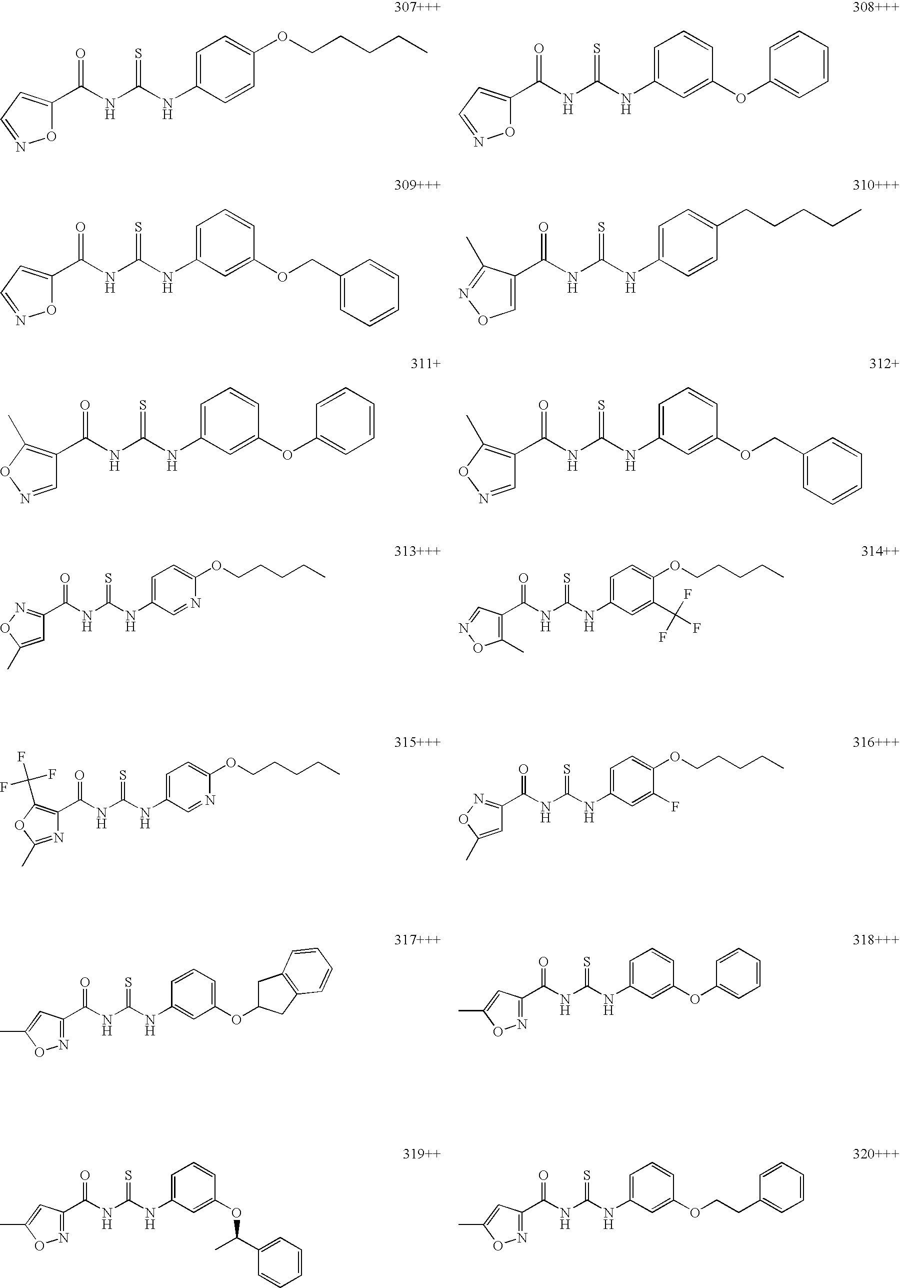

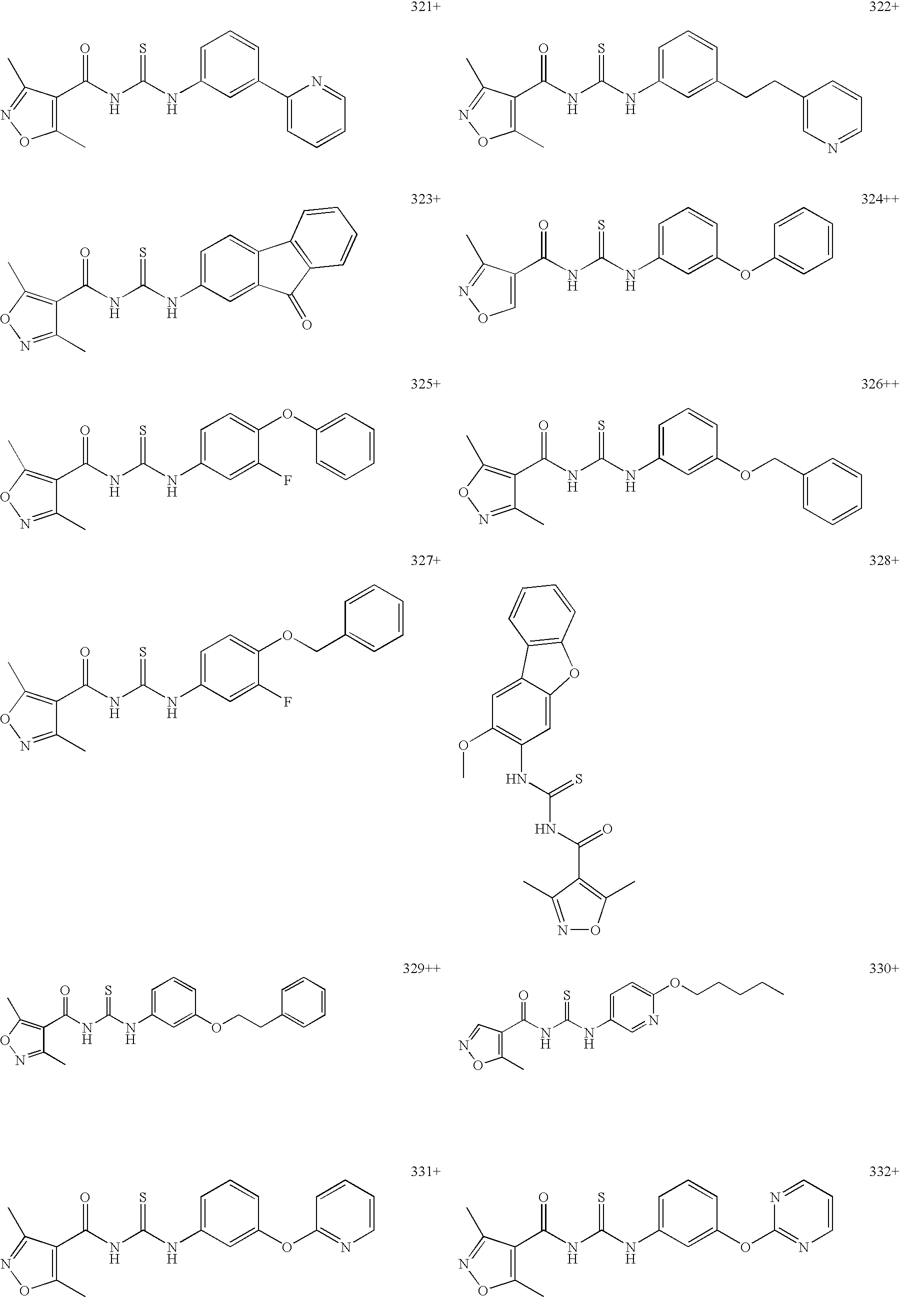

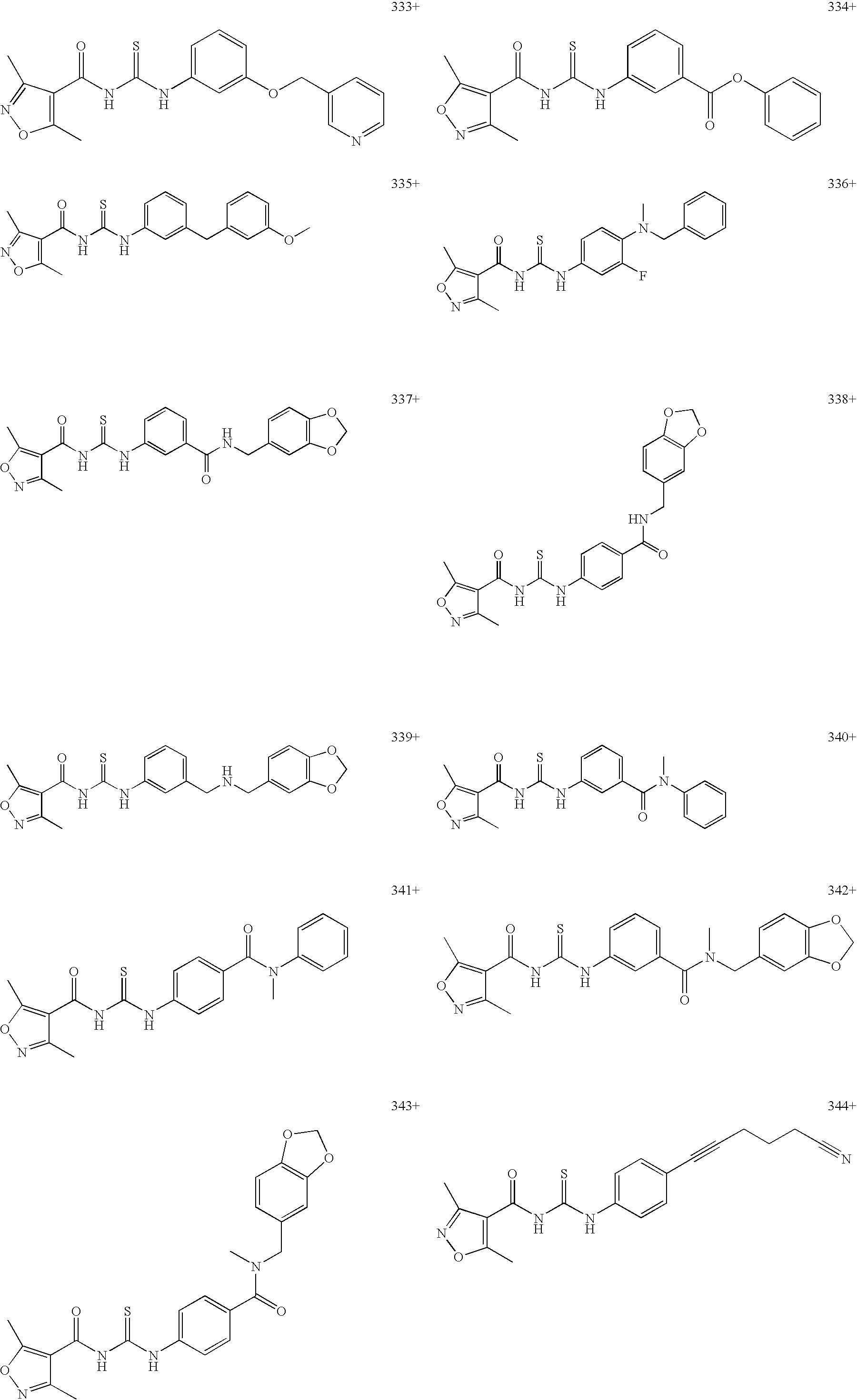

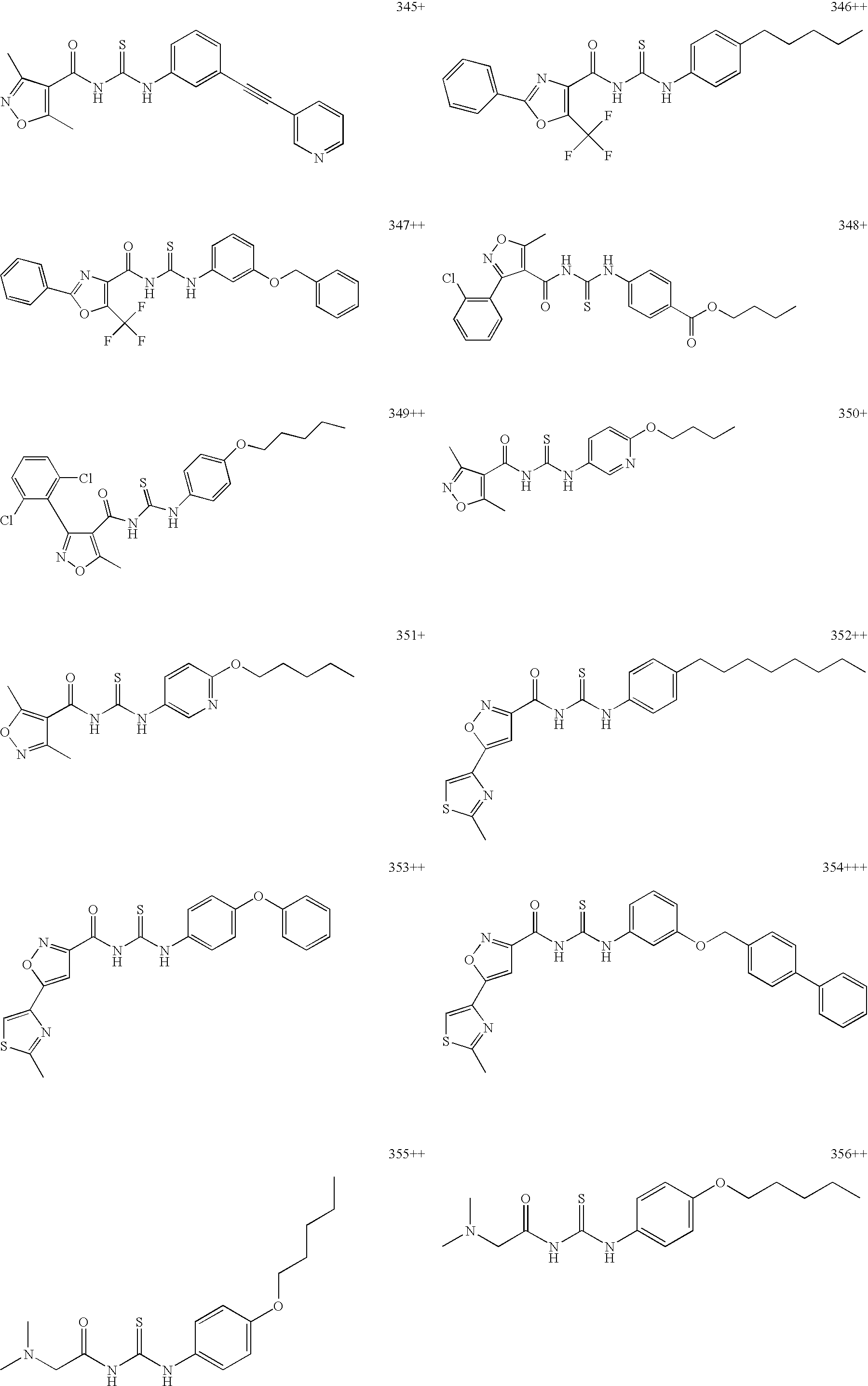

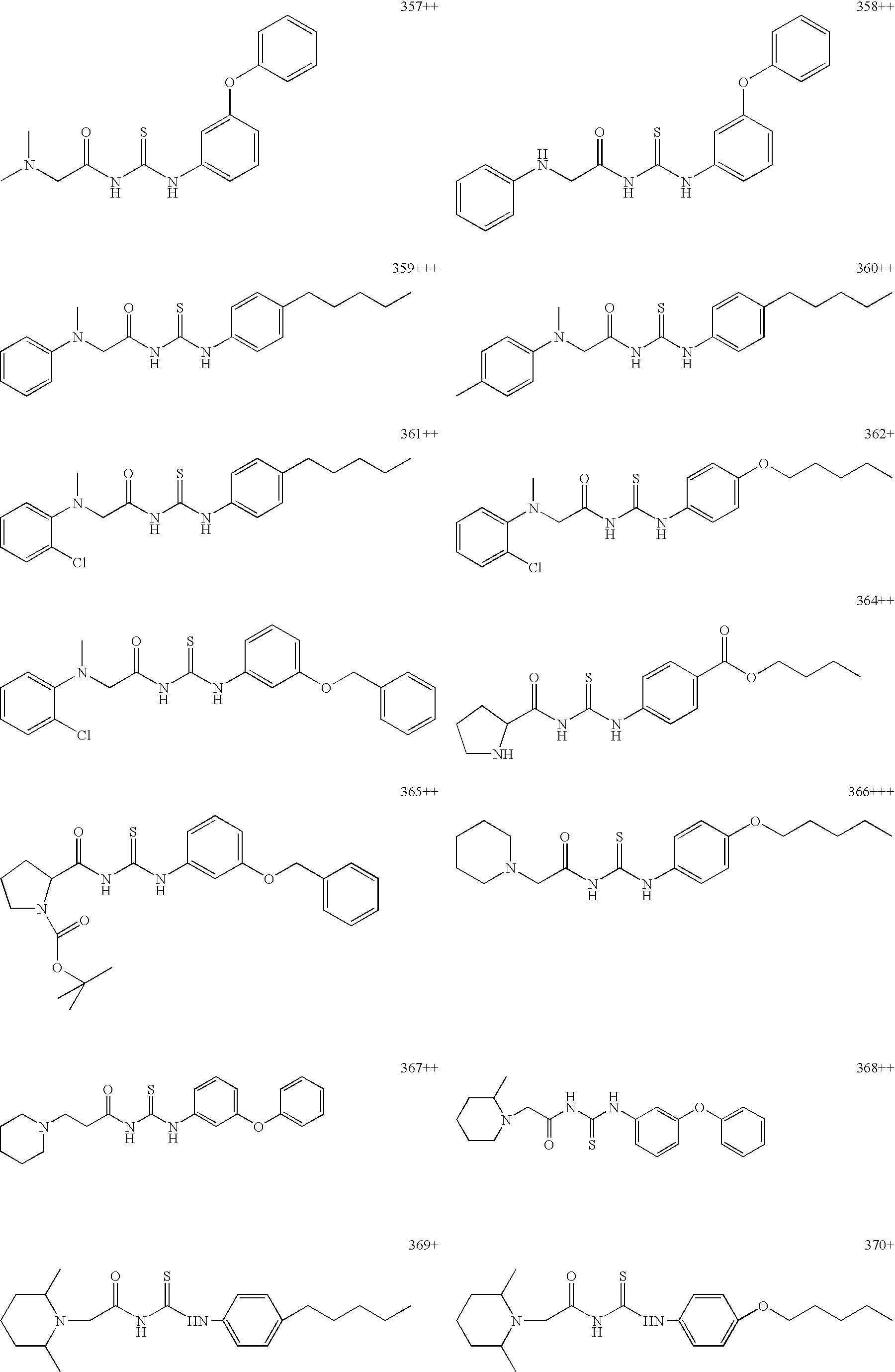

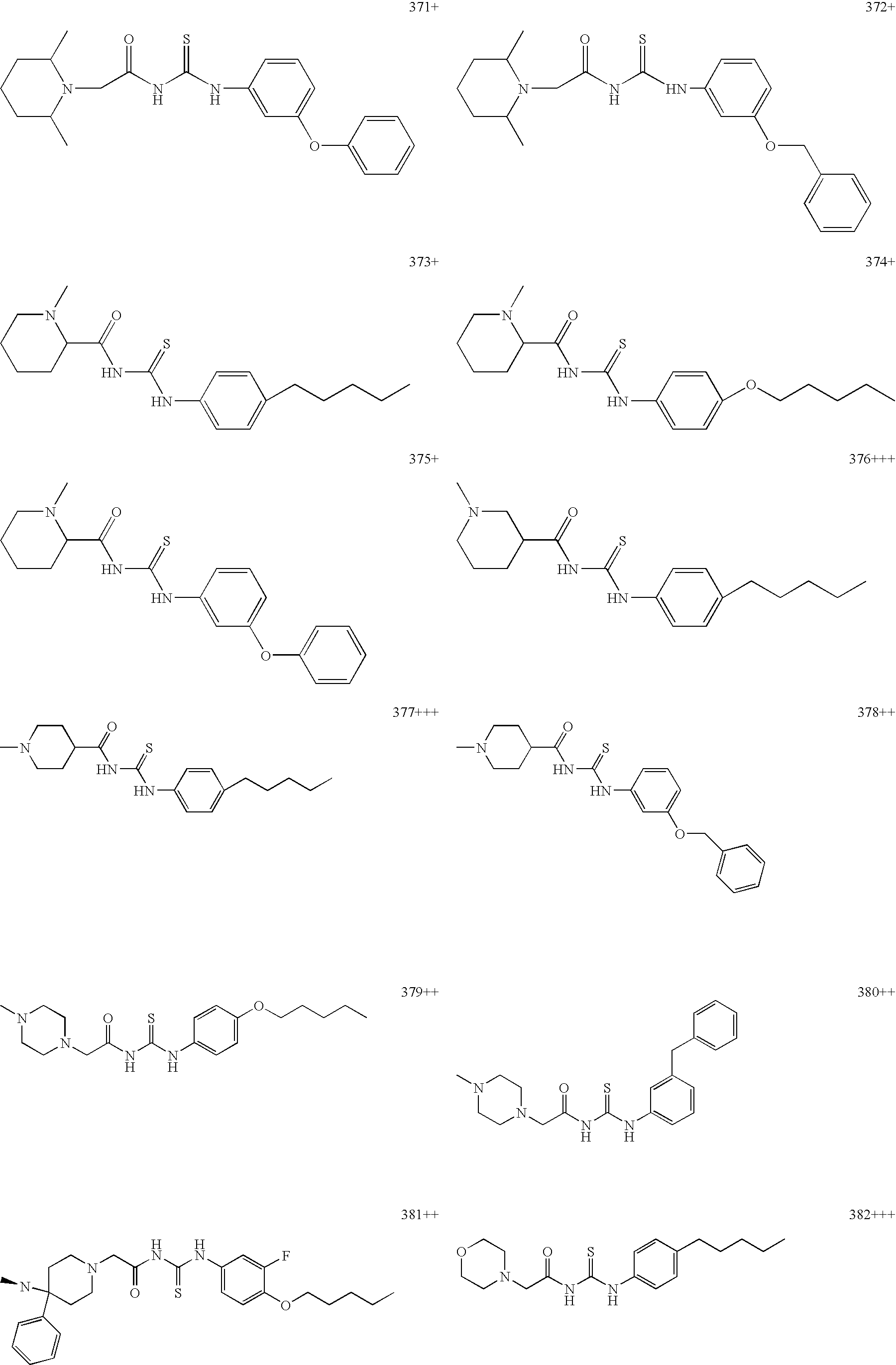

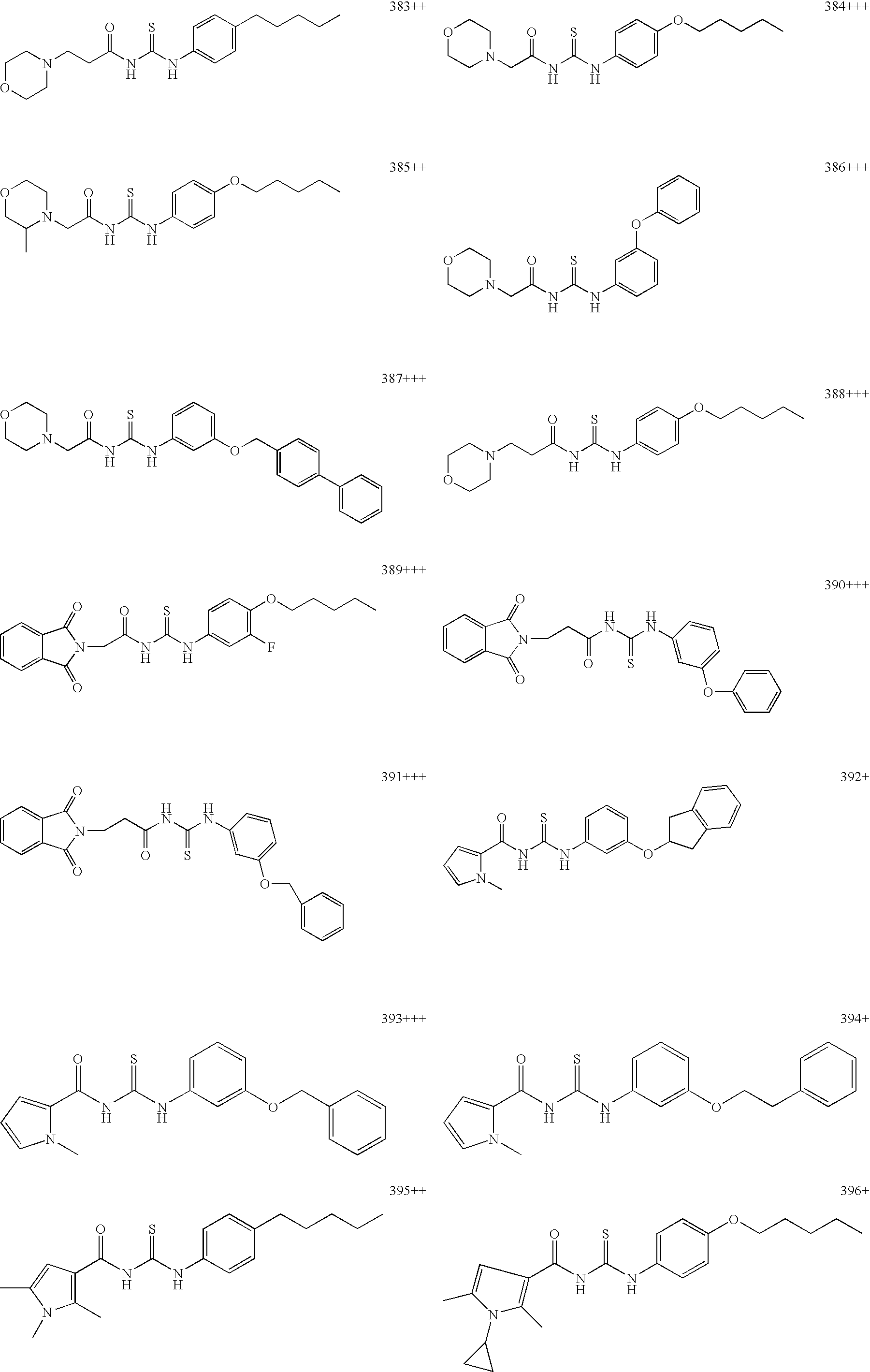

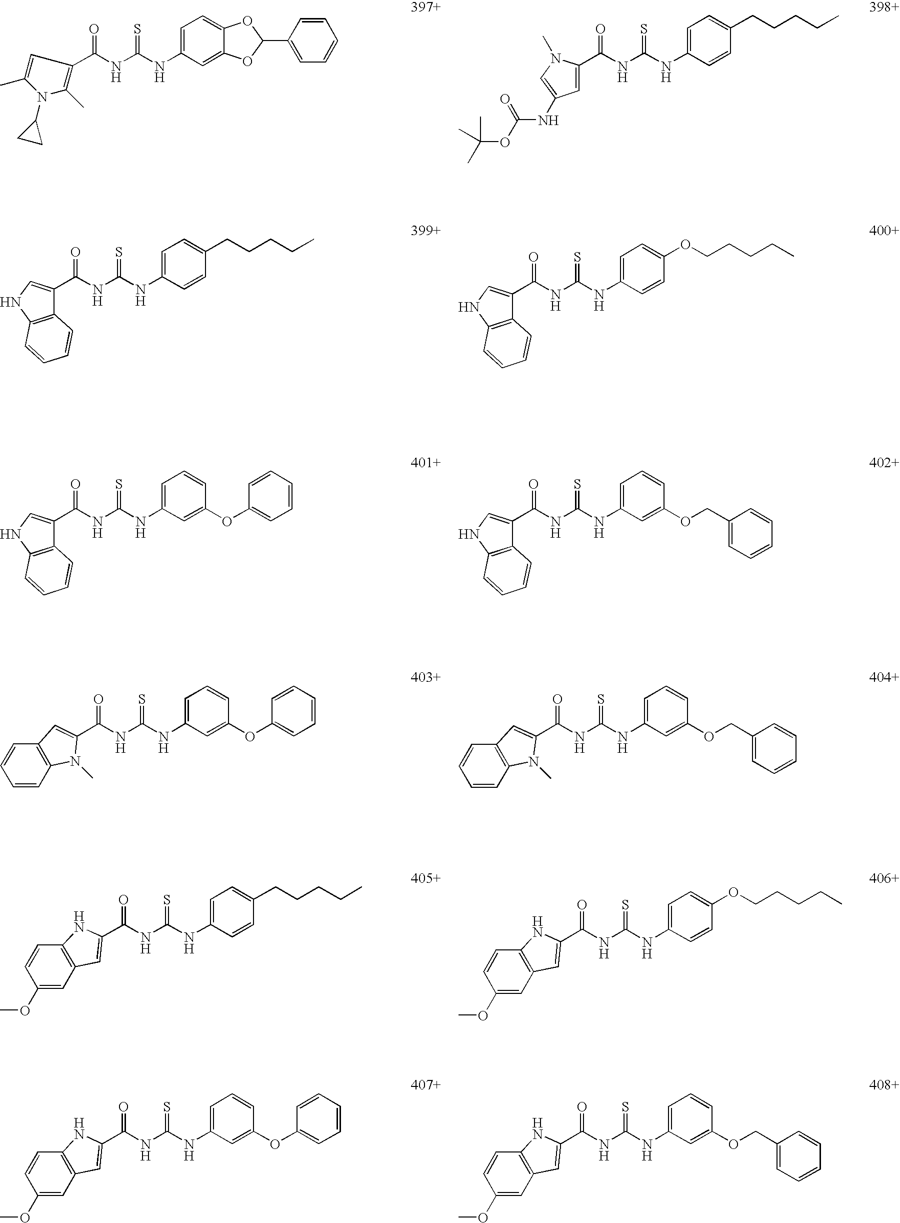

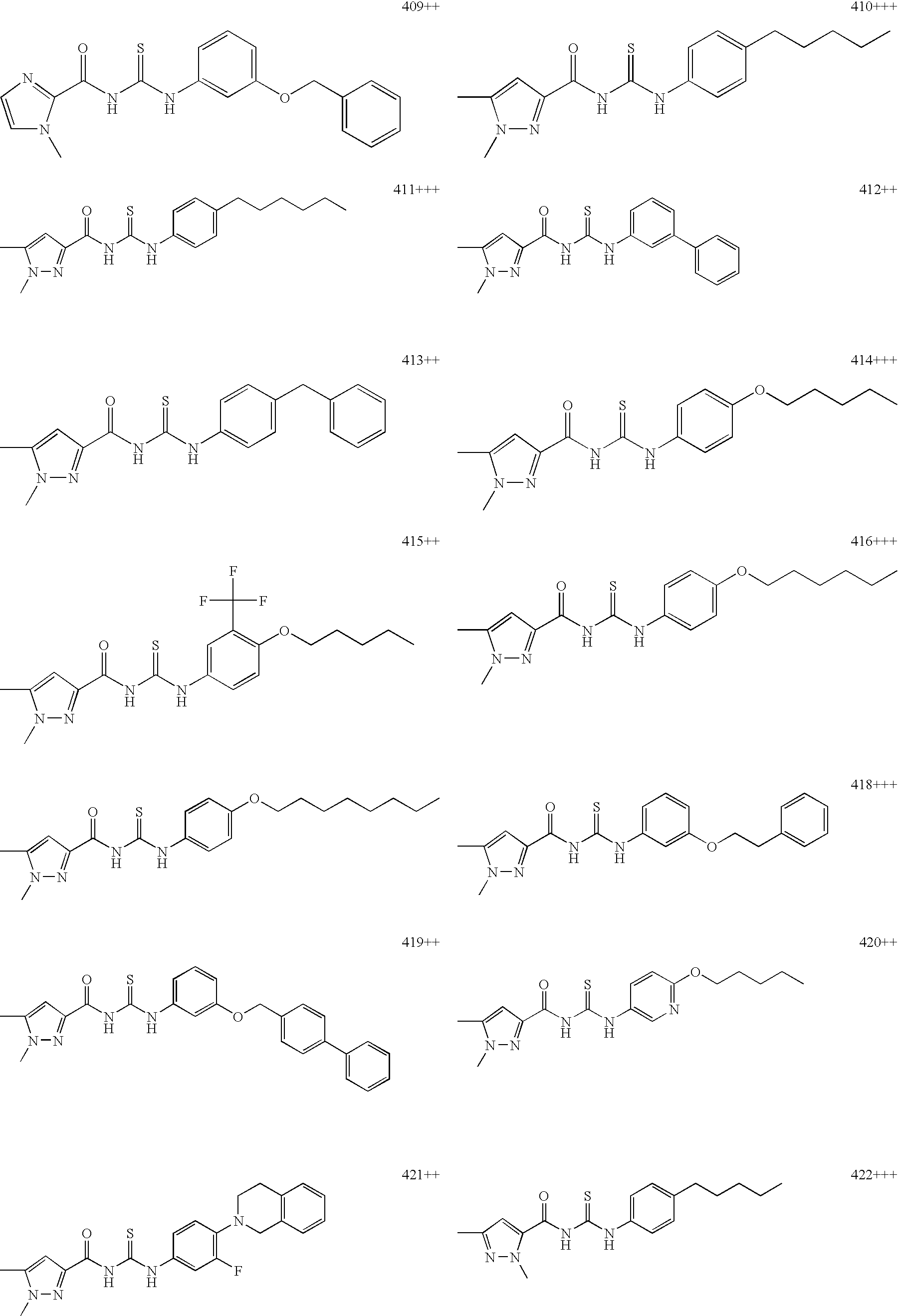

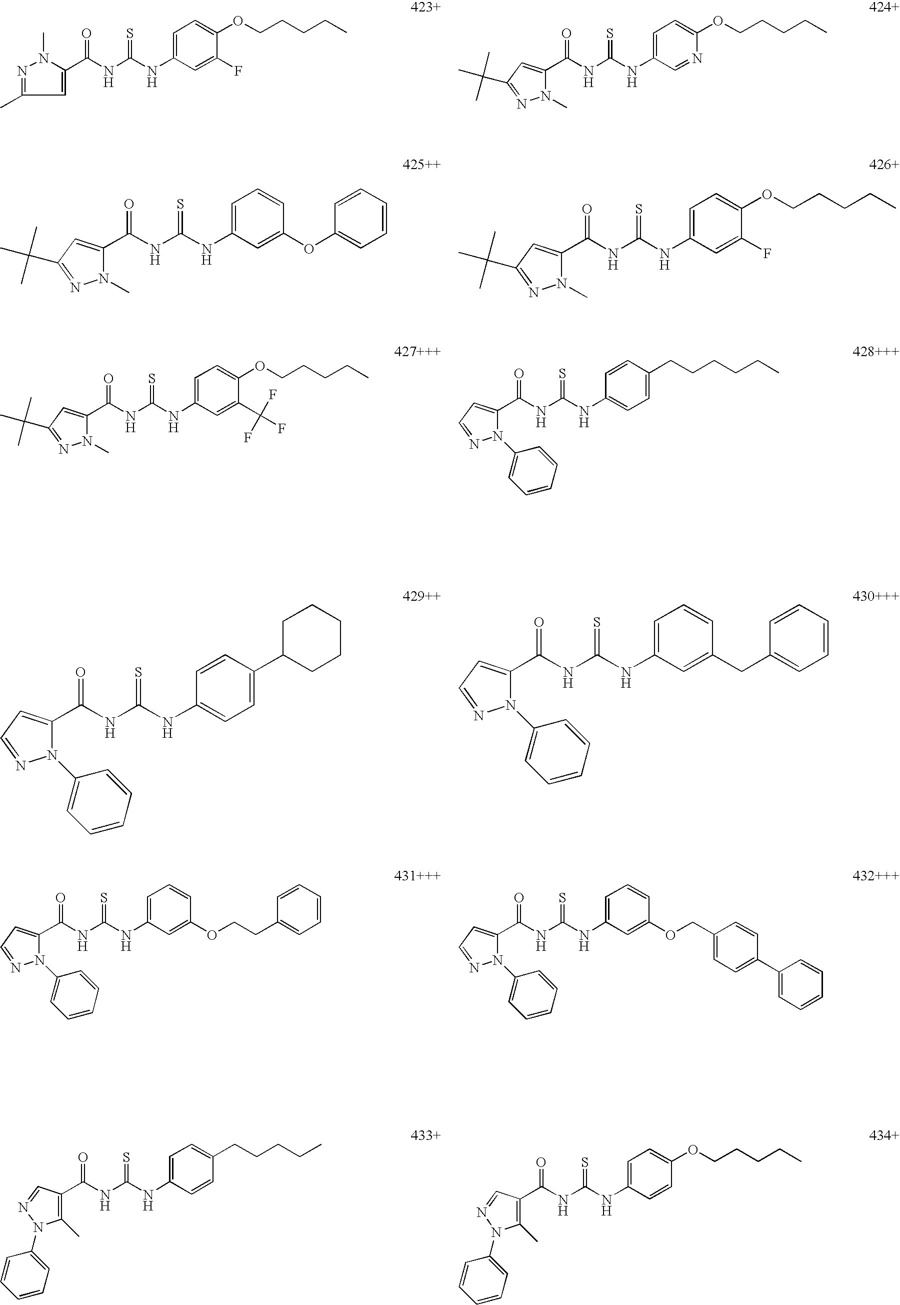

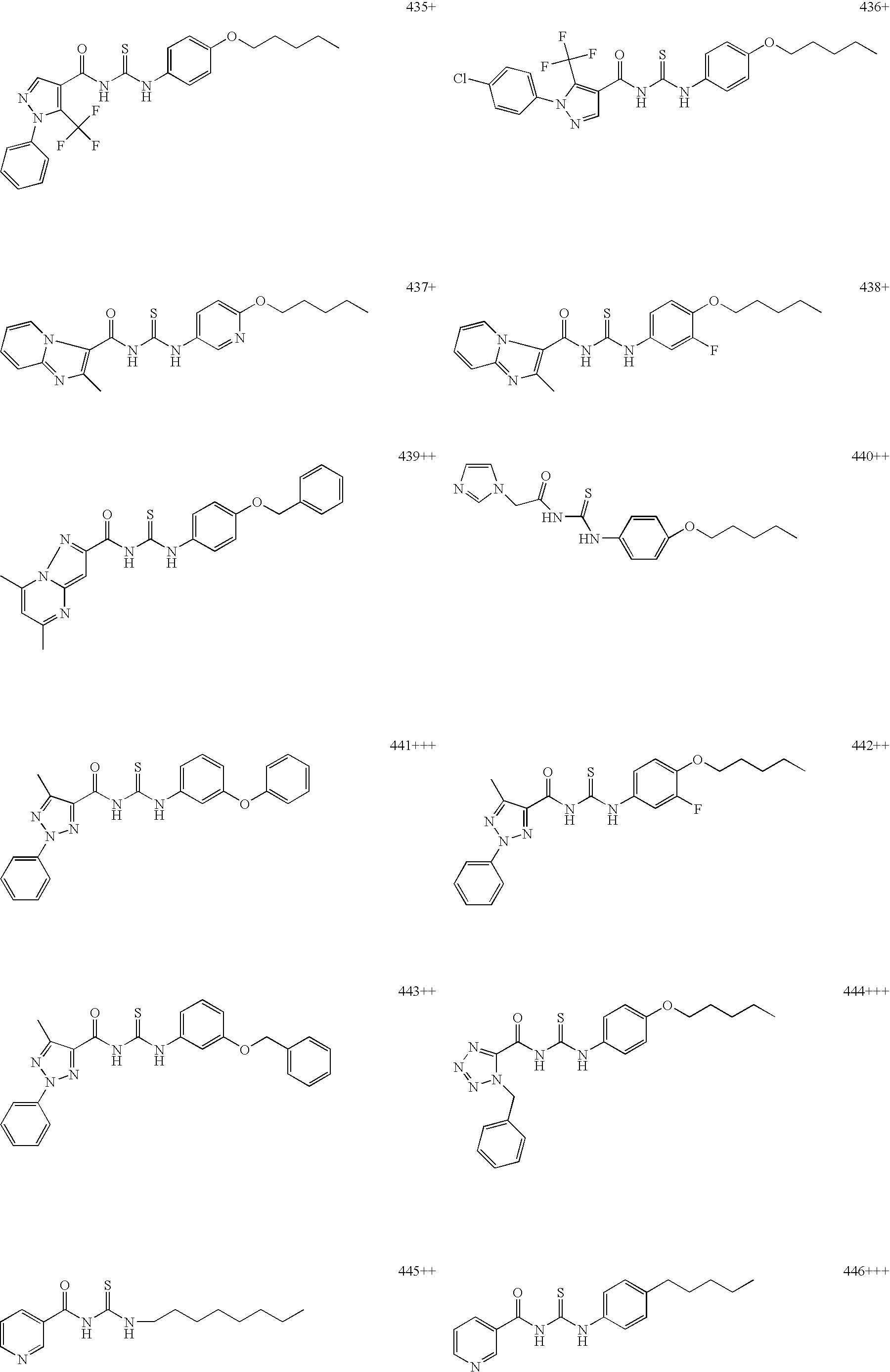

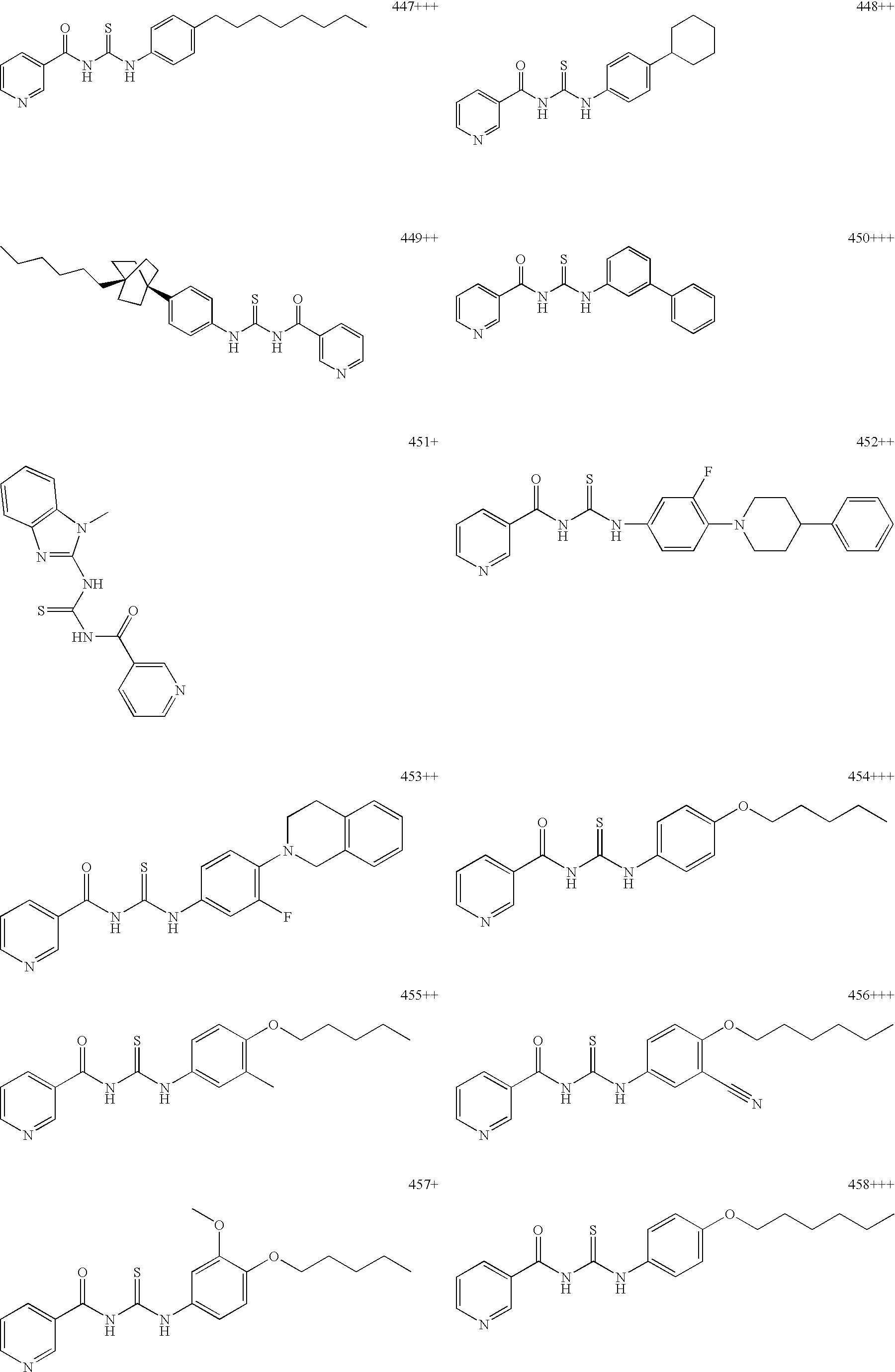

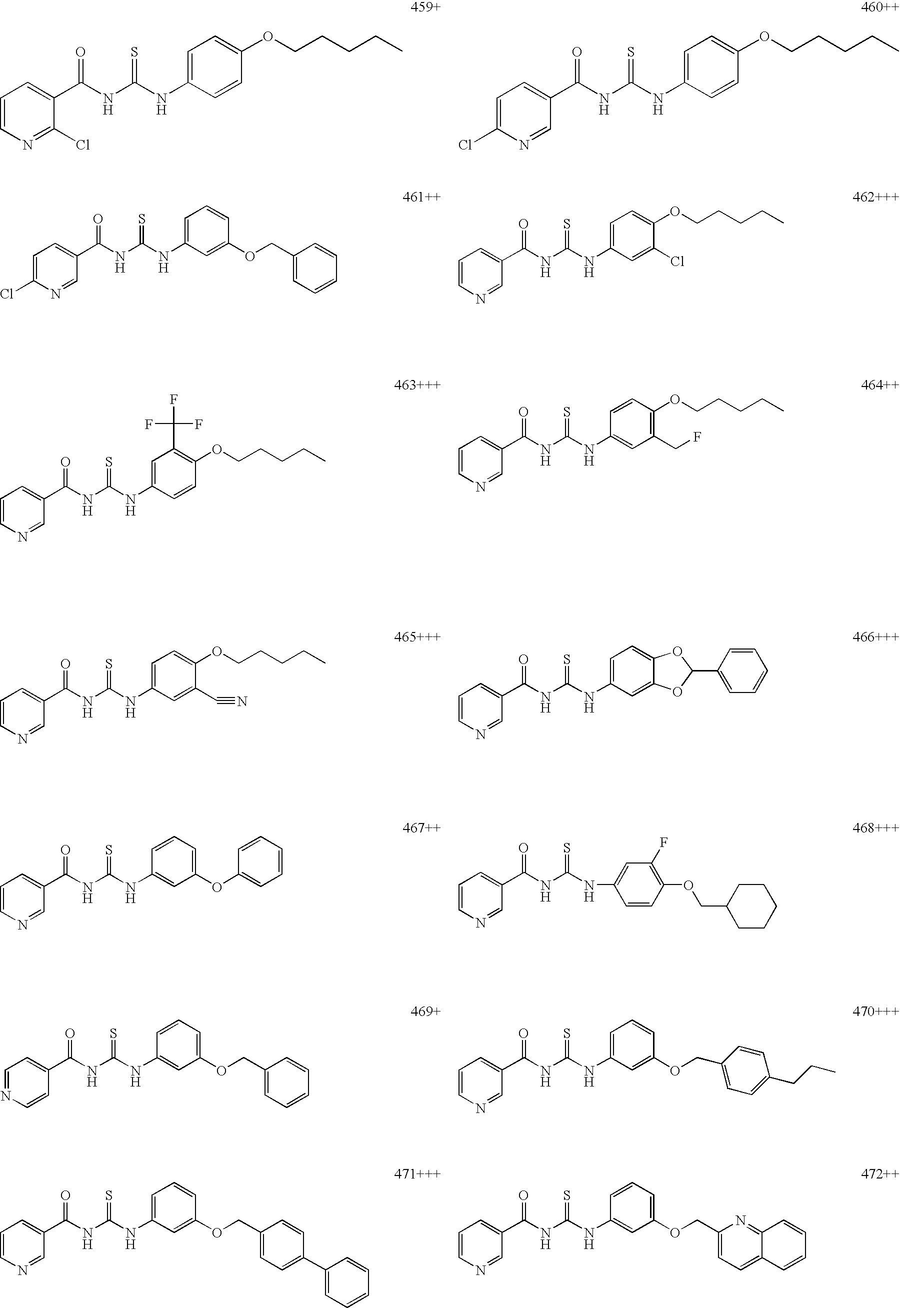

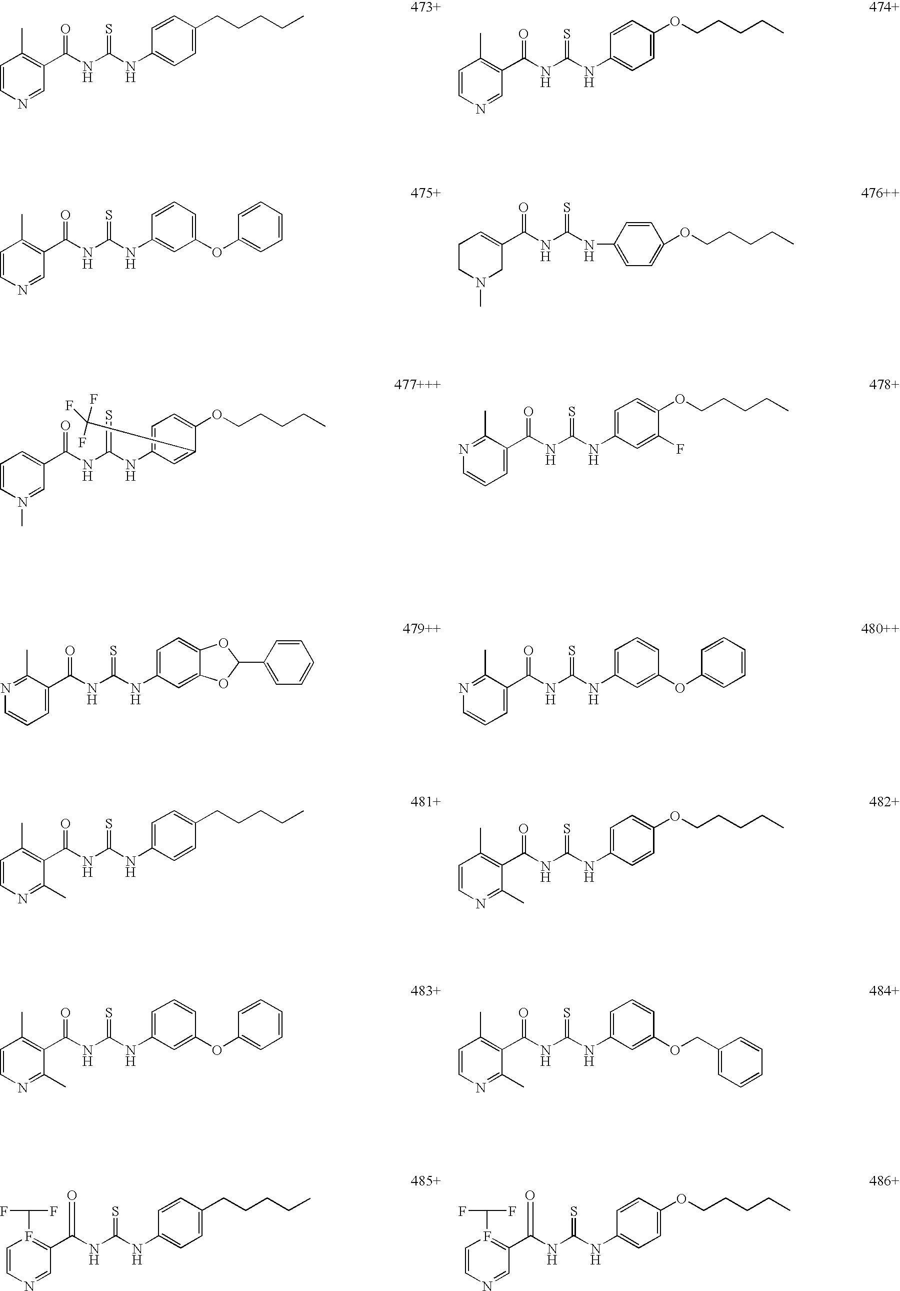

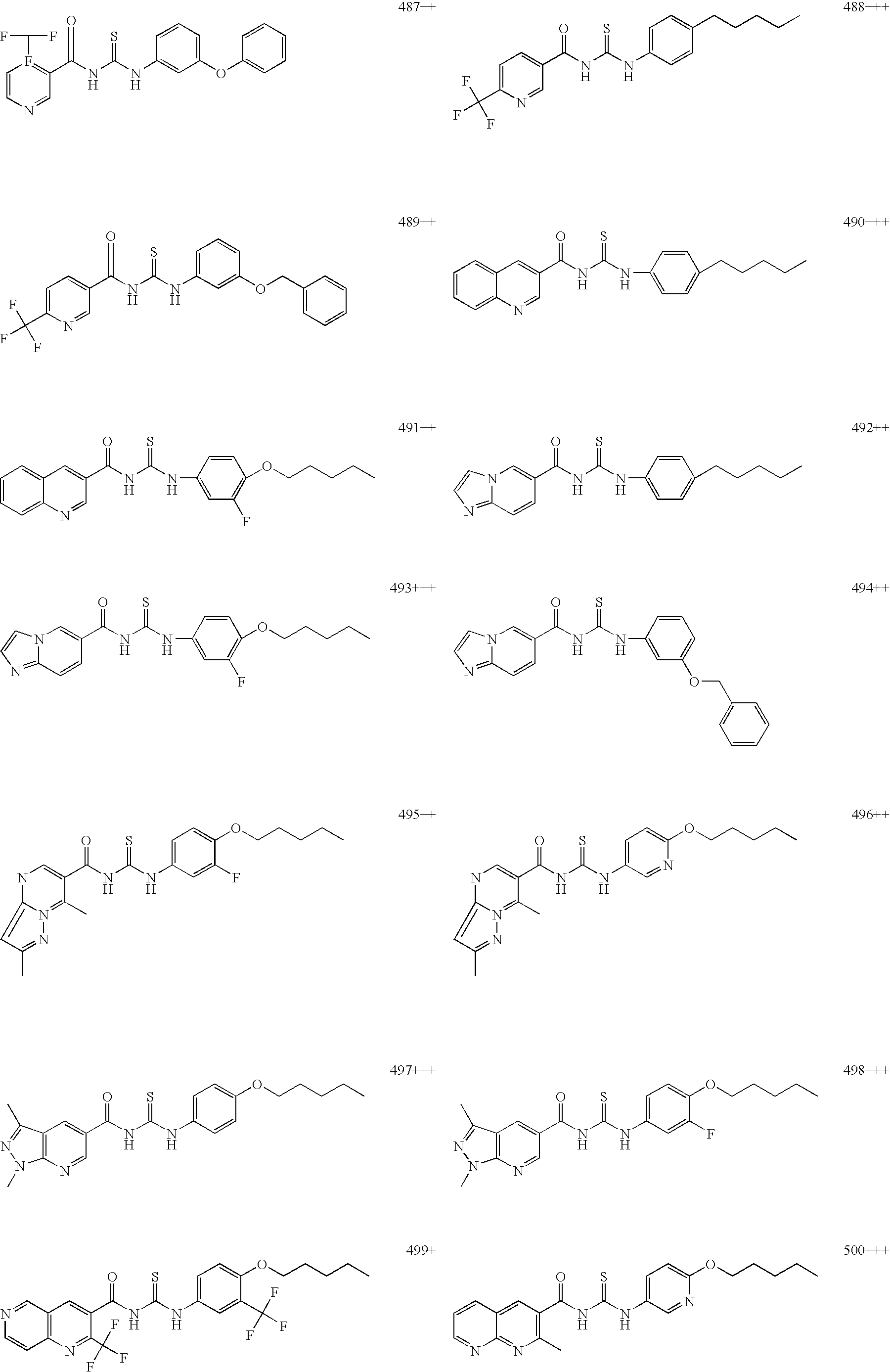

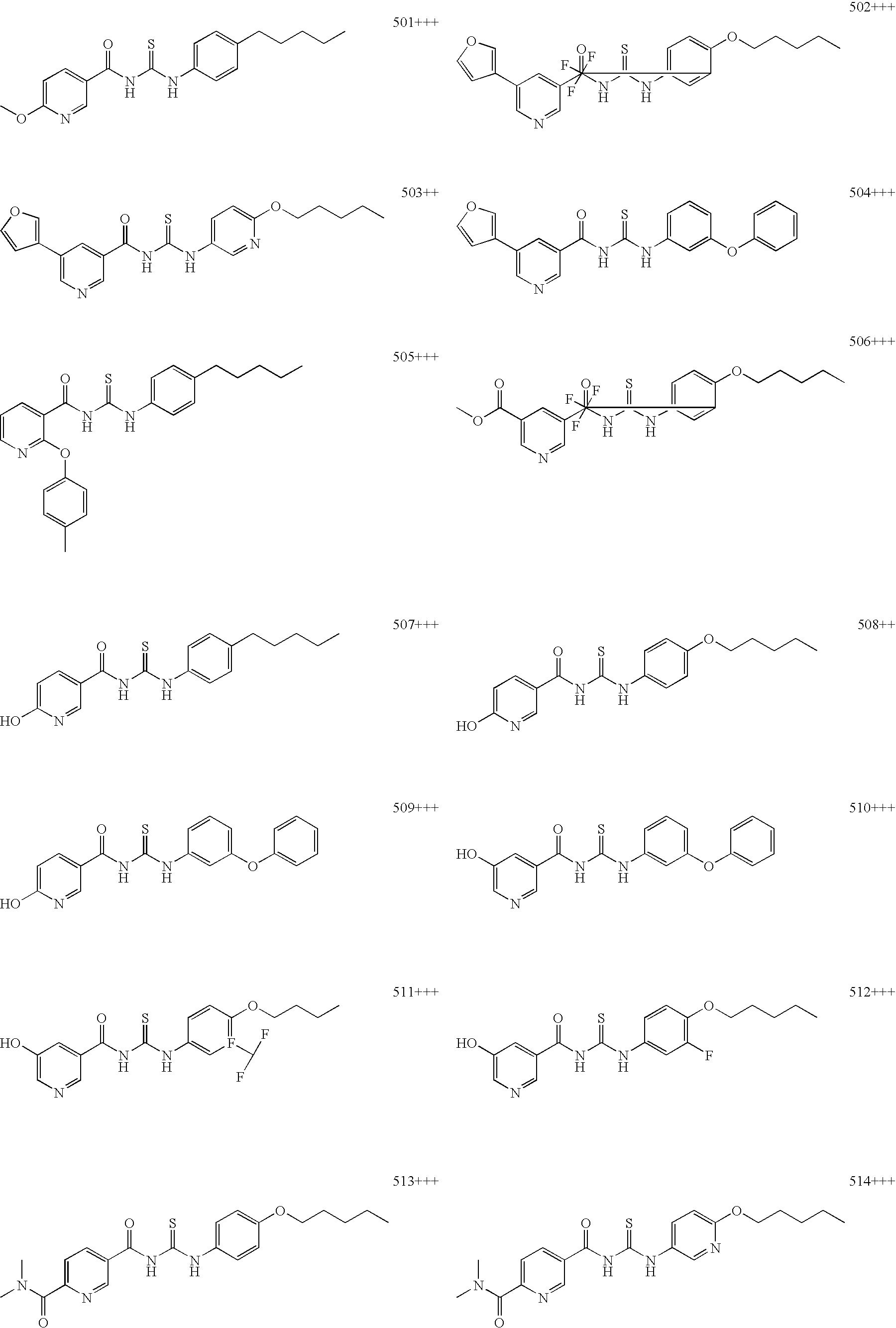

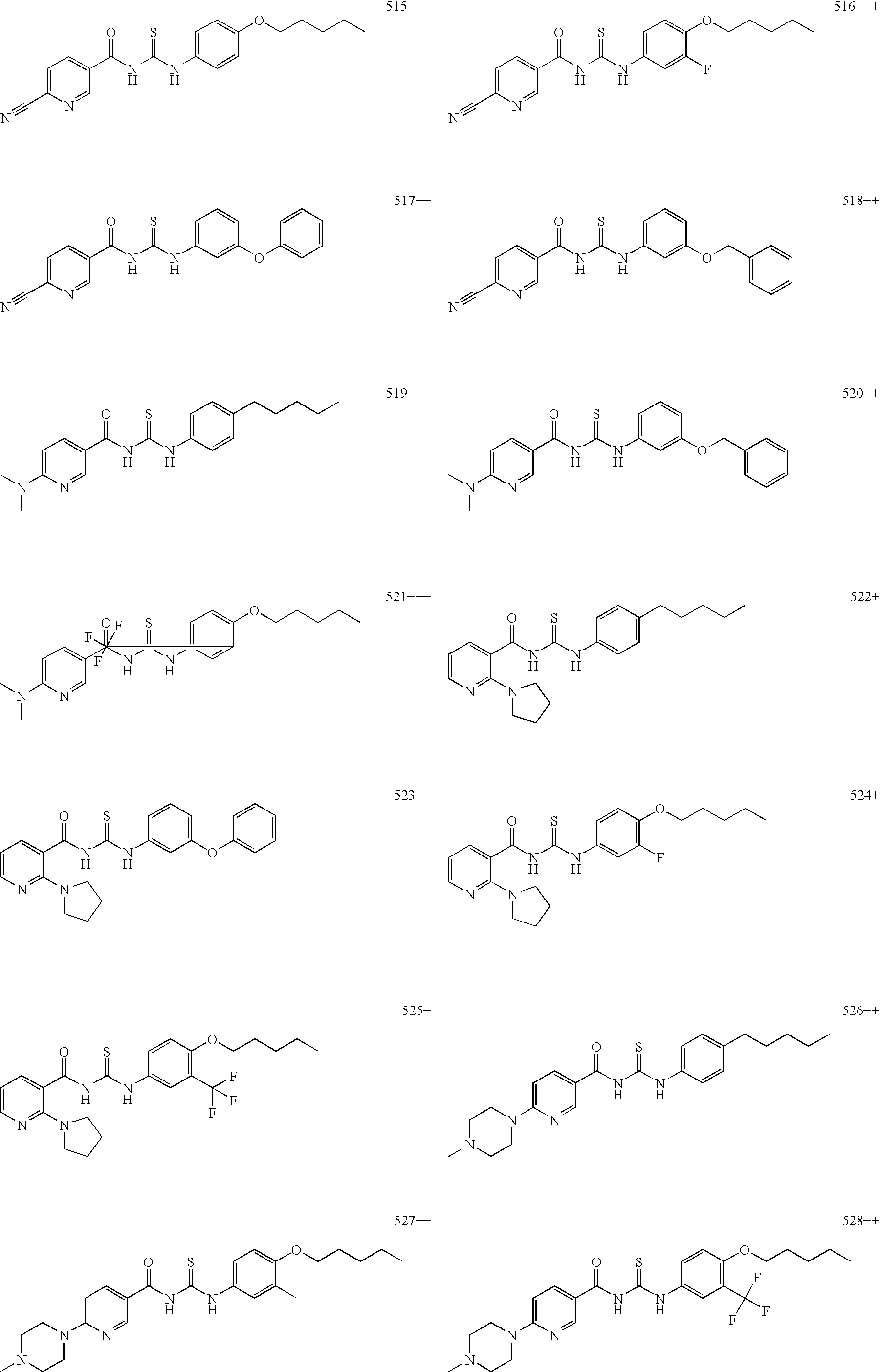

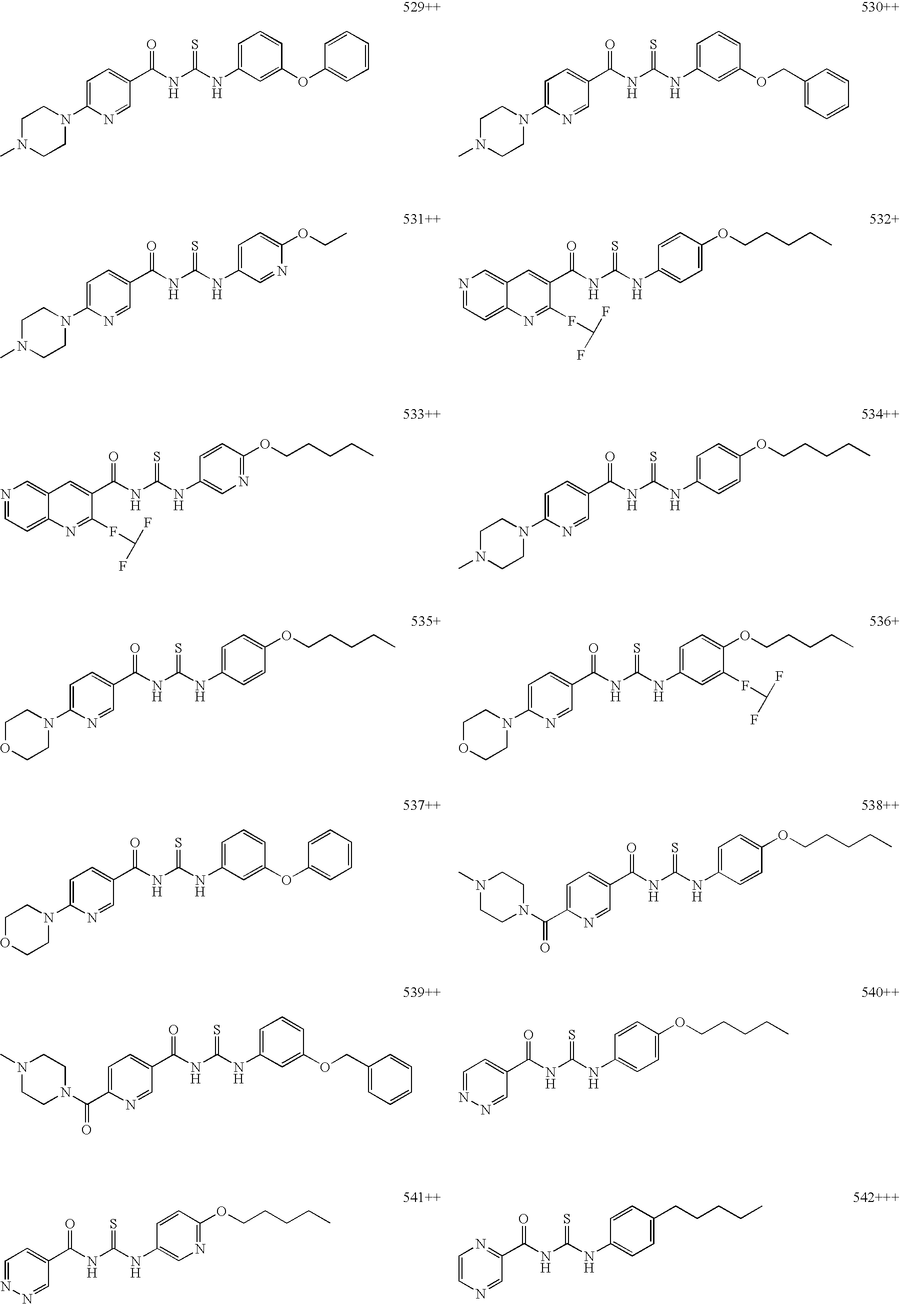

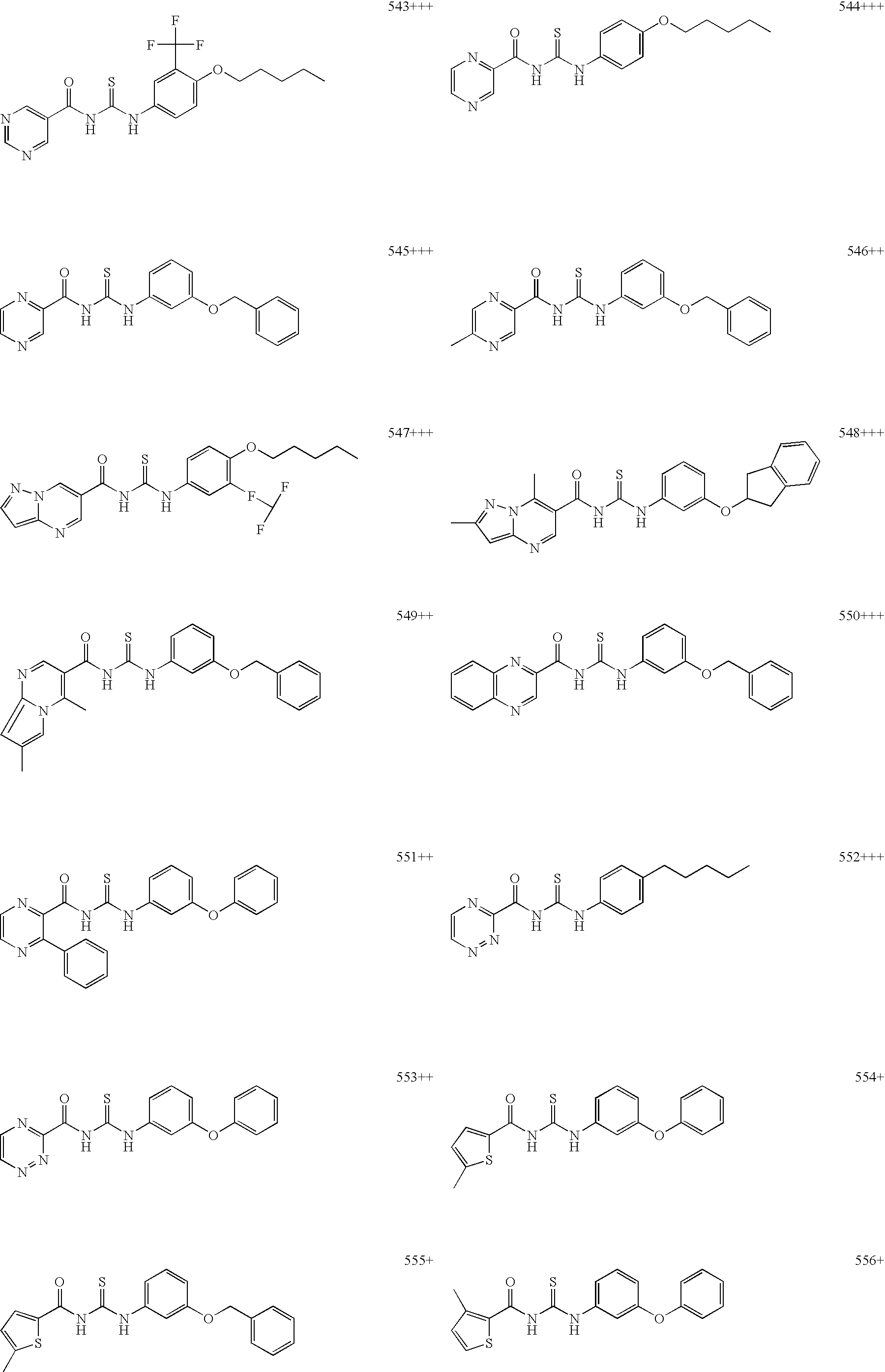

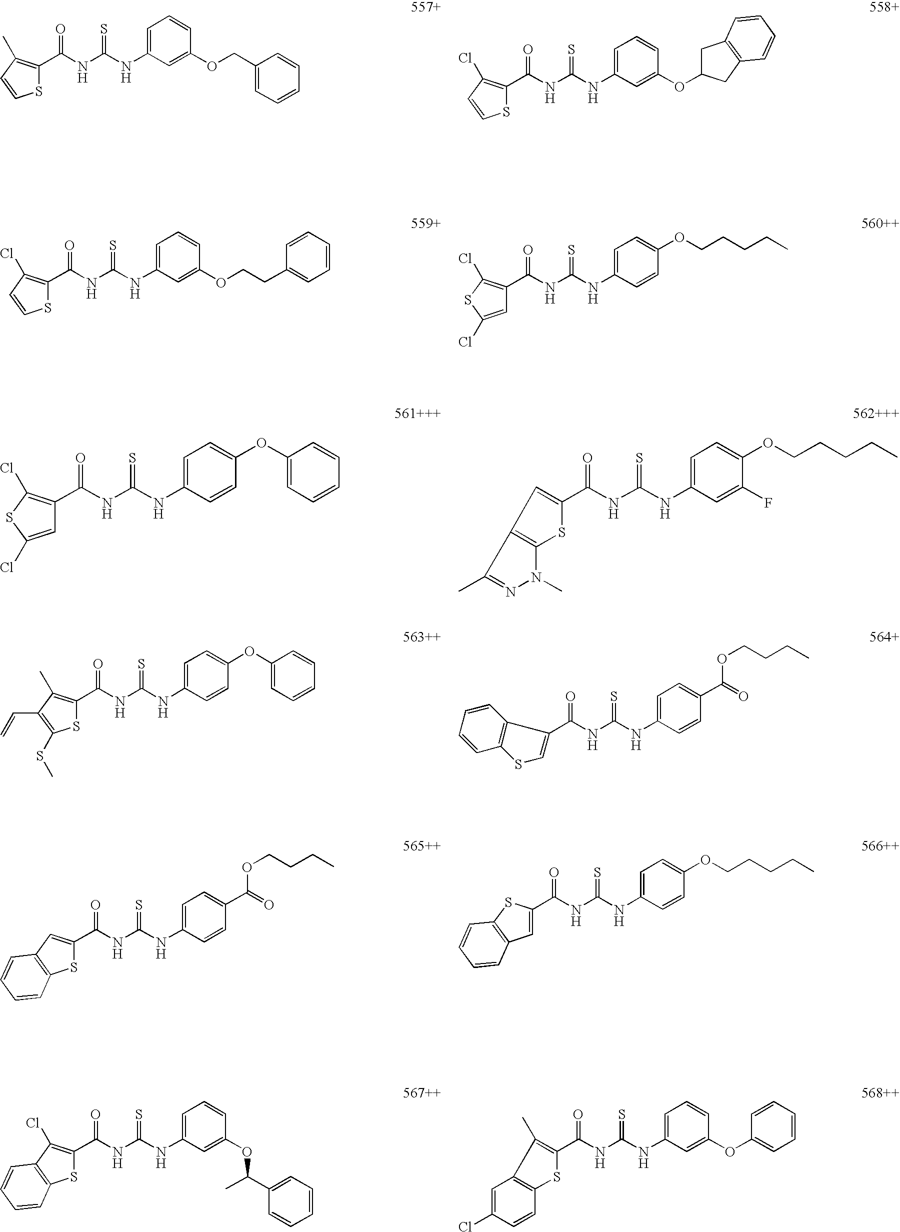

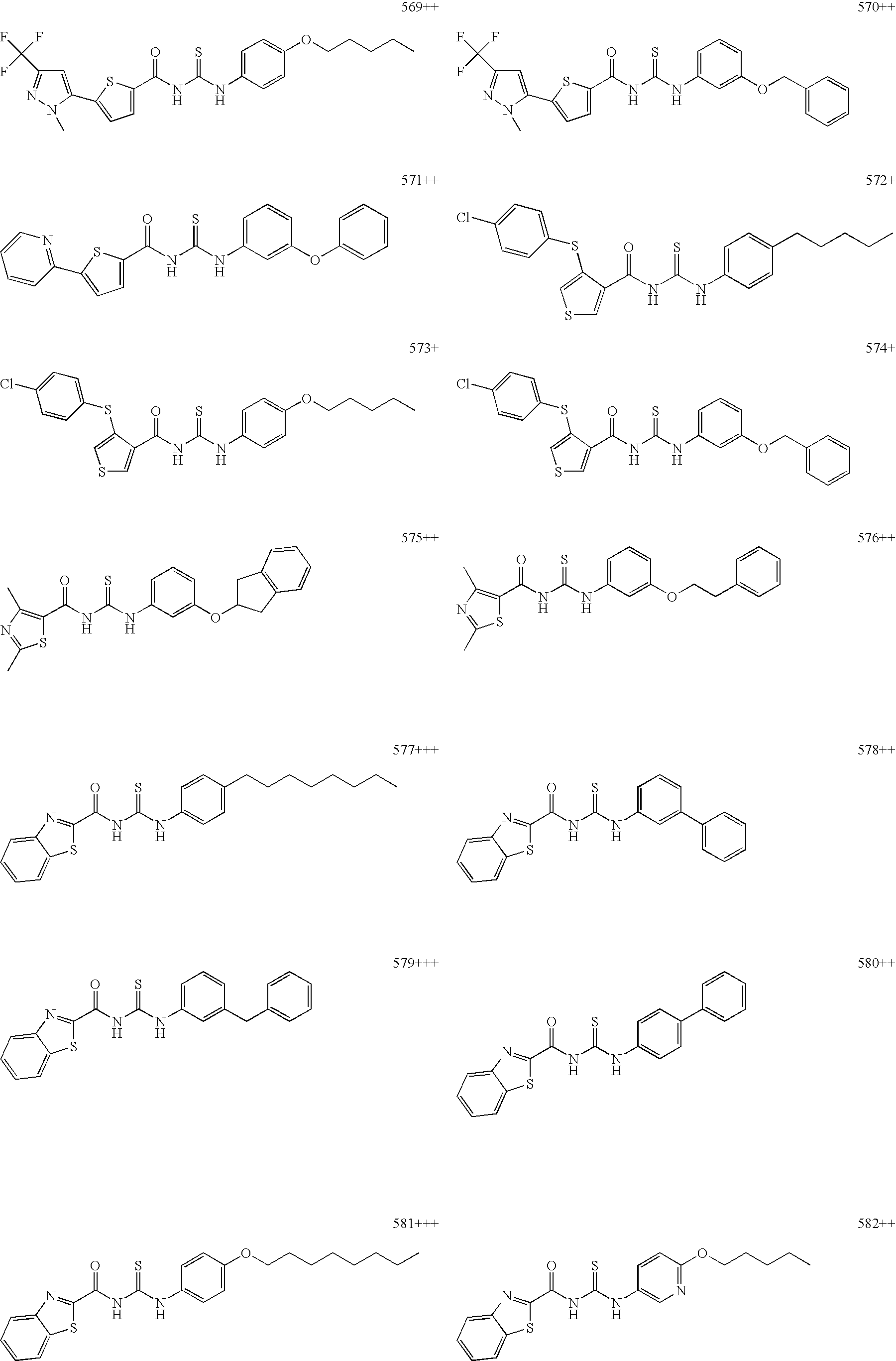

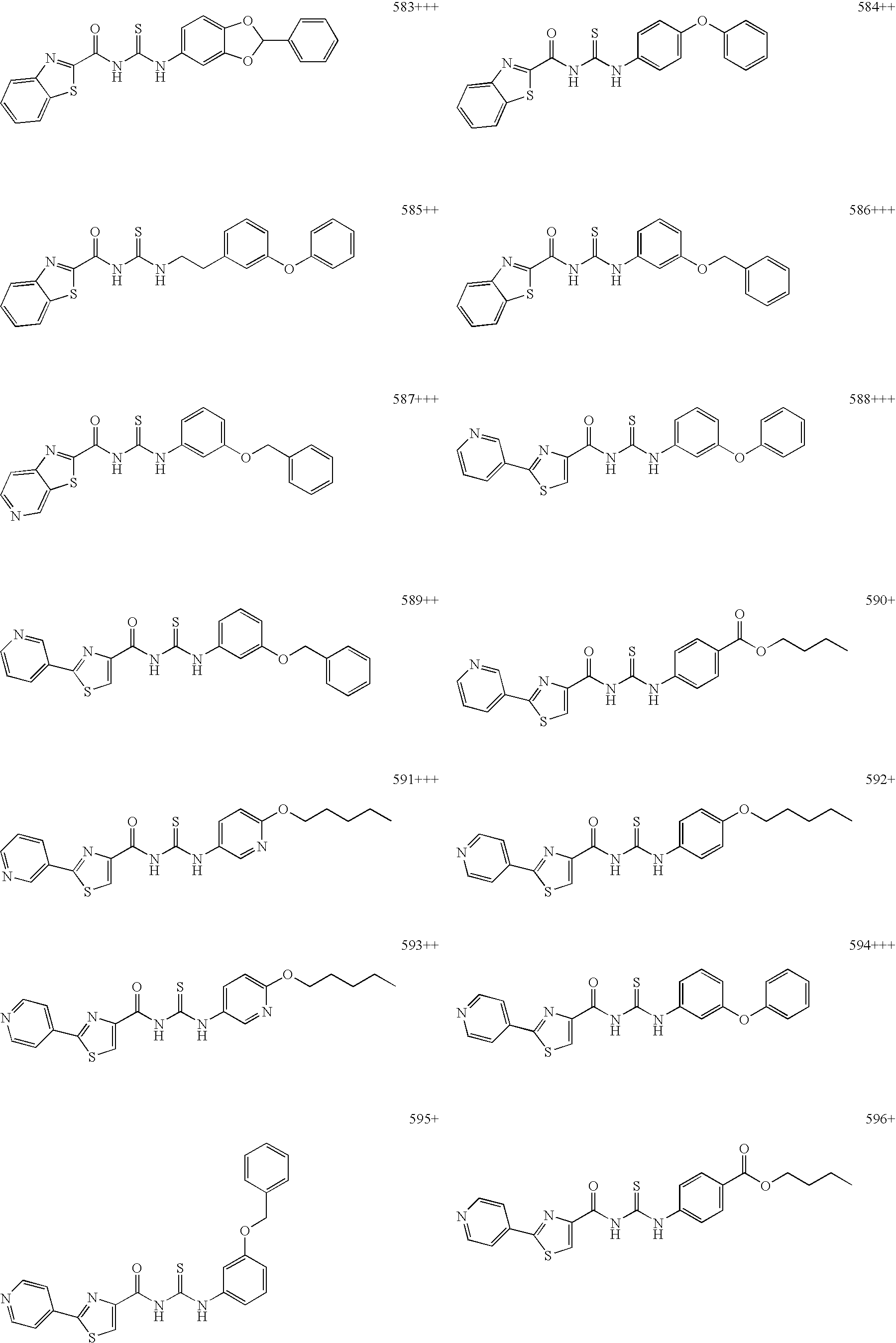

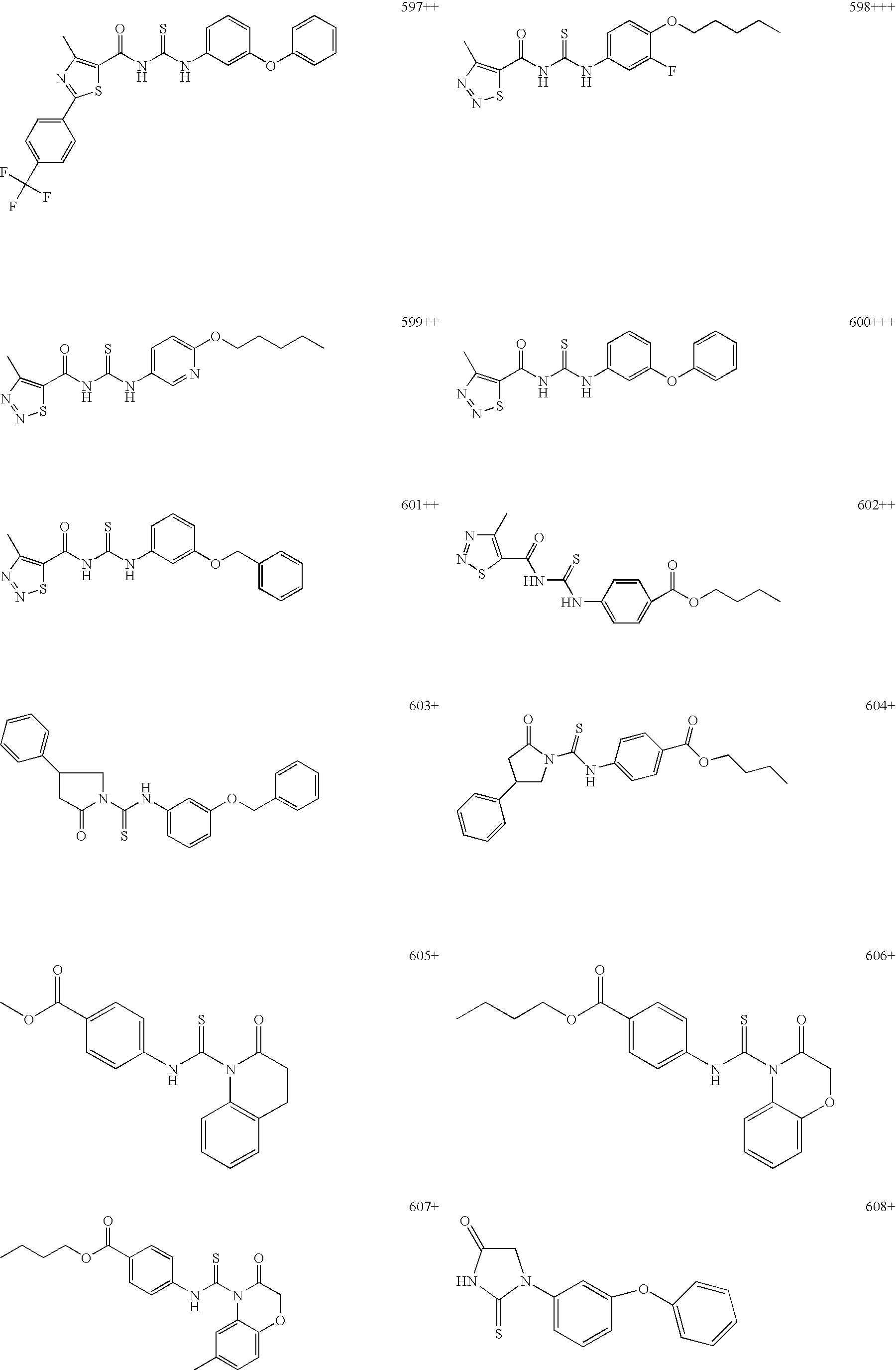

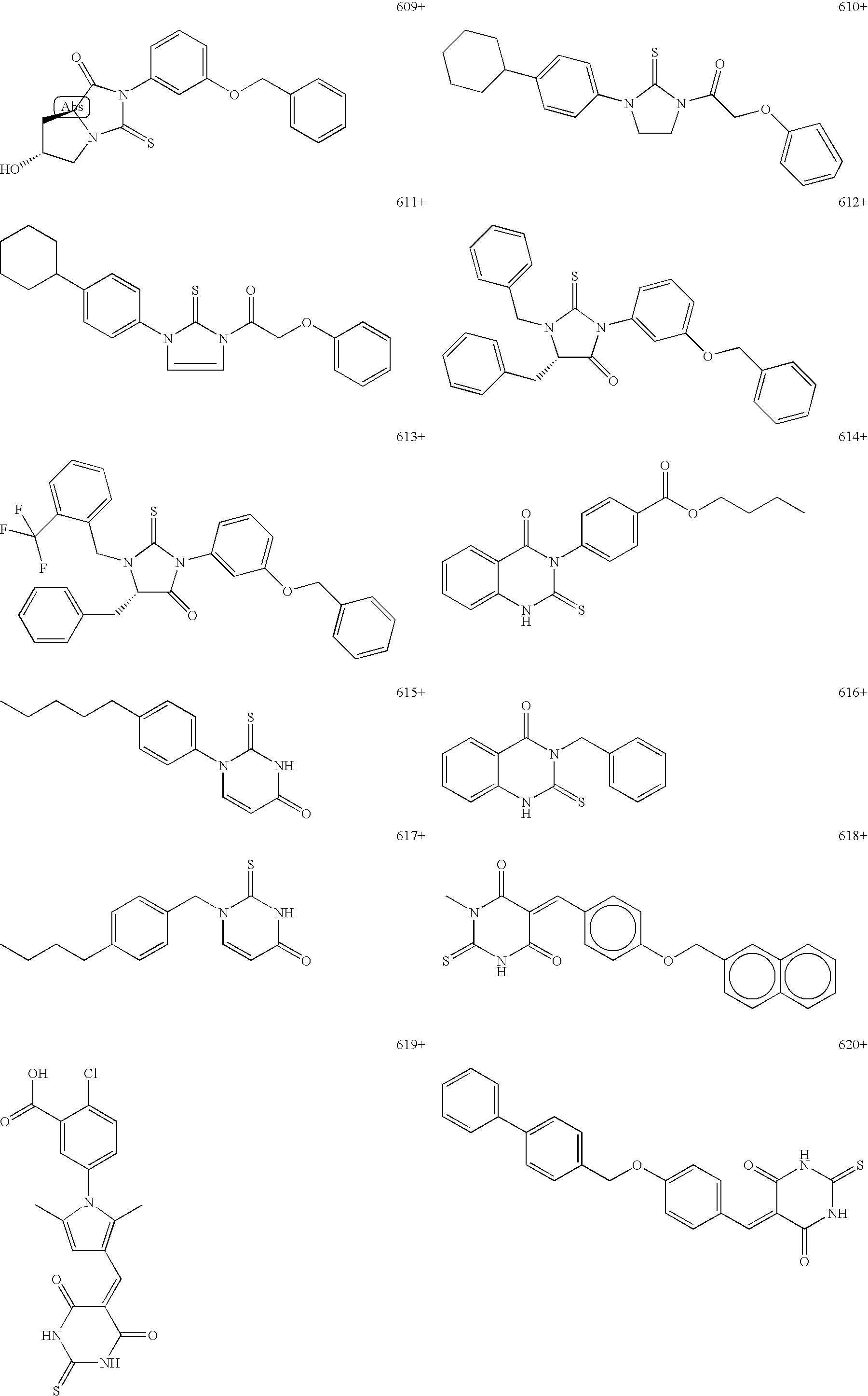

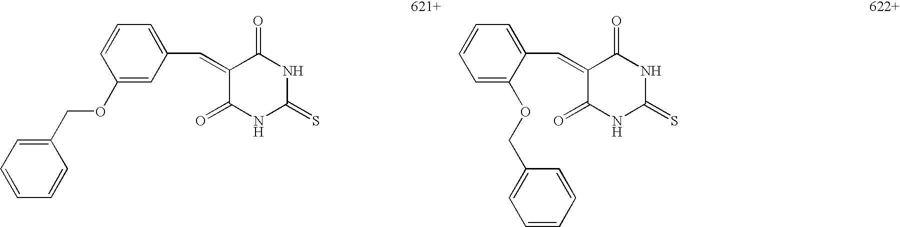

### Appendix B

| | STRUCTURE |
|---|---|
| 1 | 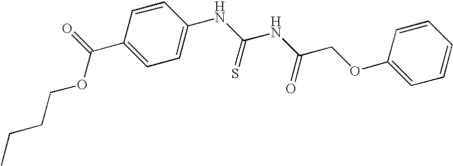 |
| 2 | 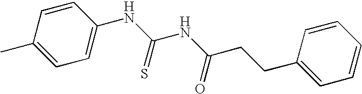 |
| 3 | 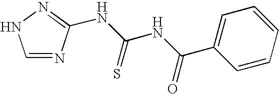 |
| 4 | 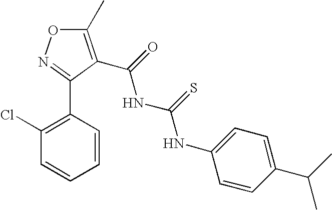 |
| 5 | 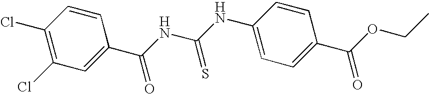 |
| 6 | 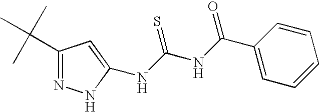 |
| 7 | 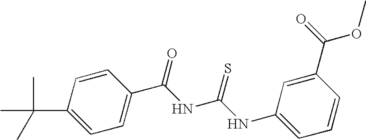 |
| 8 | 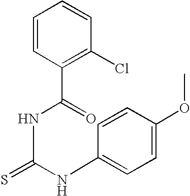 |
| 9 | 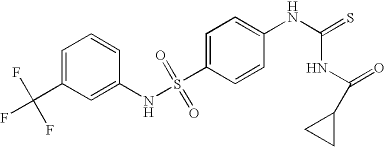 |
| 10 | 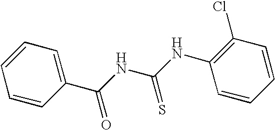 |
| 11 | 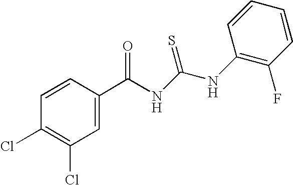 |
| 12 | 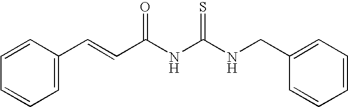 |
| 13 | 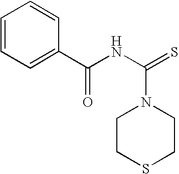 |
| 14 | 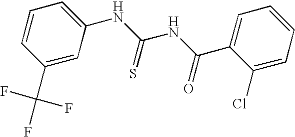 |
| 15 | 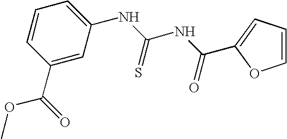 |
| 16 | 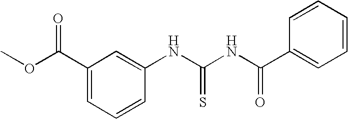 |
| 17 | 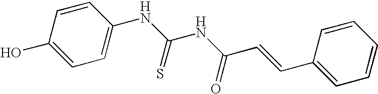 |
| 18 | 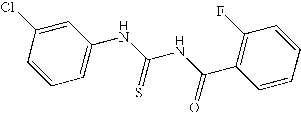 |
| 19 | 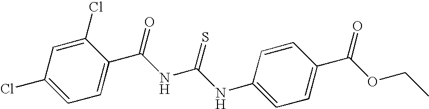 |
| 20 | 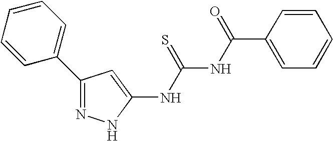 |
| 21 | 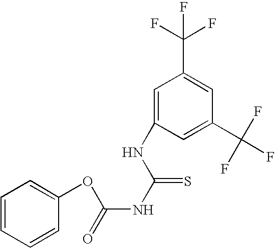 |
| 22 | 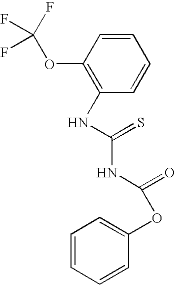 |
| 23 | 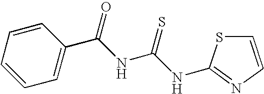 |
| 24 | 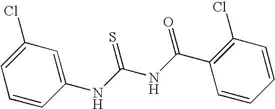 |
| 25 | 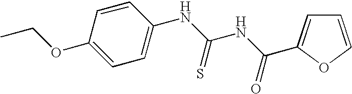 |
| 26 | 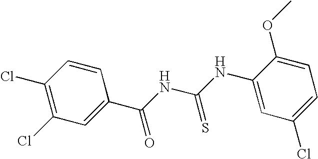 |
| 27 | 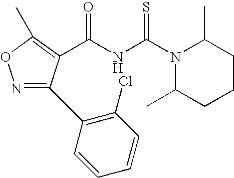 |
| 28 | 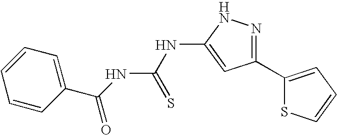 |
| 29 | 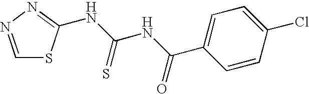 |
| 30 | 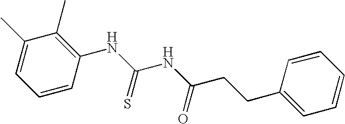 |
| 31 | 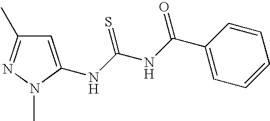 |
| 32 | 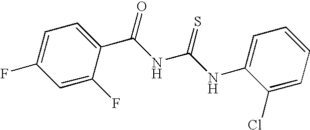 |
| 33 | 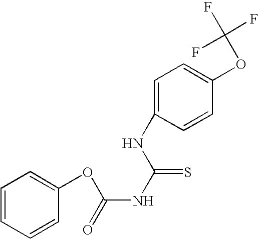 |
| 34 | 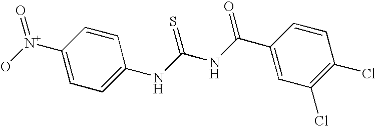 |
| 35 | 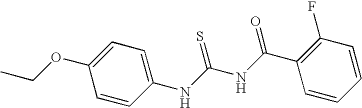 |
| 36 | 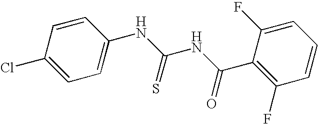 |
| 37 | 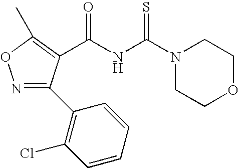 |
| 38 | 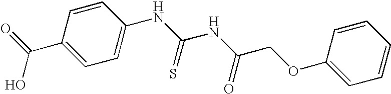 |
| 39 | 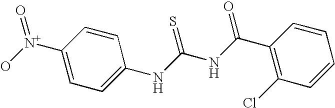 |
| 40 | 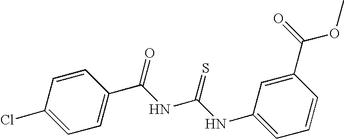 |
| 41 | 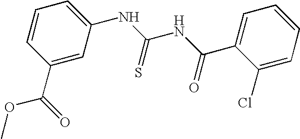 |
| 42 | 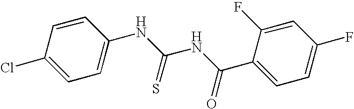 |
| 43 | 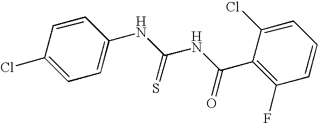 |
| 44 | 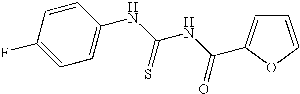 |
| 45 | 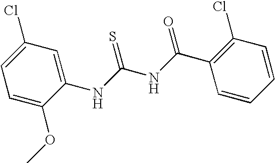 |
| 46 | 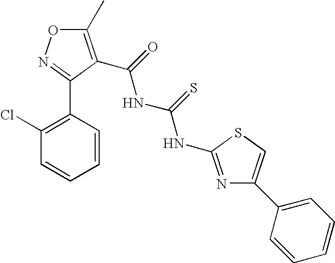 |
| 47 | 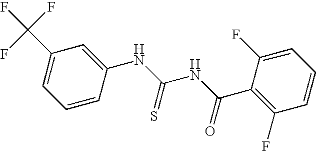 |
| 48 | 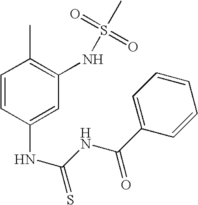 |
| 49 | 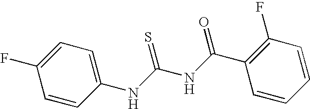 |
| 50 | 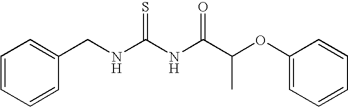 |
| 51 | 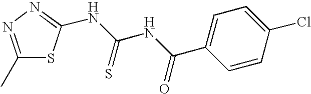 |
| 52 | 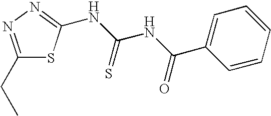 |
| 53 | 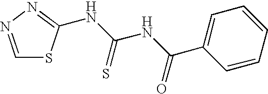 |
| 54 | 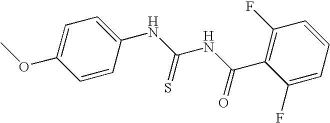 |
| 55 | 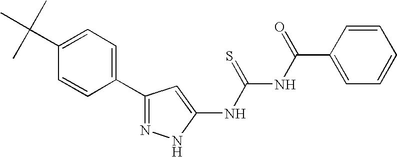 |
| 56 | 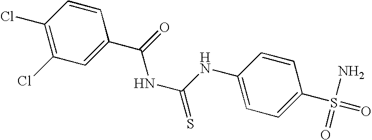 |
| 57 | 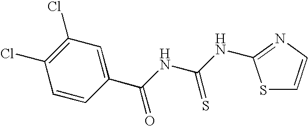 |
| 58 | 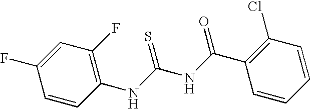 |
| 59 | 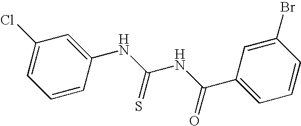 |
| 60 | 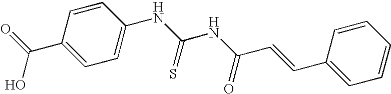 |
| 61 | 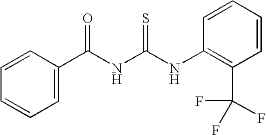 |
| 62 | 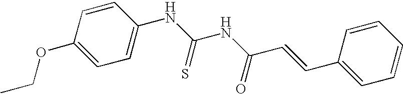 |
| 63 | 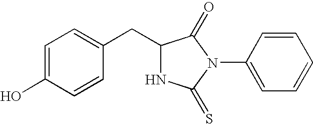 |
| 64 | 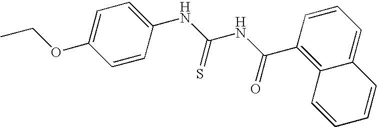 |
| 65 | 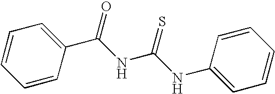 |
| 66 | 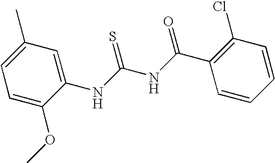 |
| 67 | 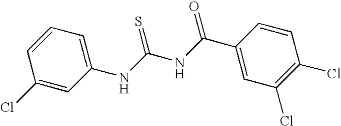 |
| 68 | 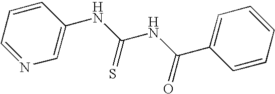 |
| 69 | 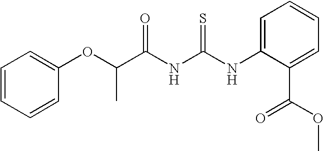 |
| 70 | 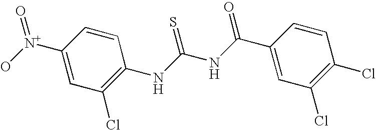 |
| 71 | 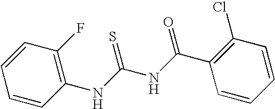 |
| 72 | 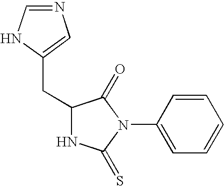 |
| 73 | 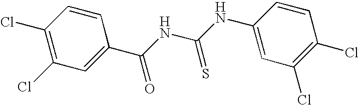 |
| 74 | 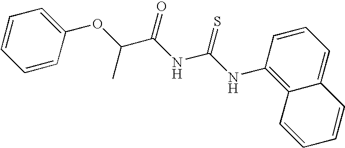 |
| 75 | 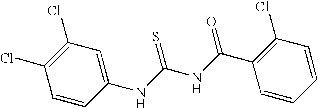 |
| 76 | 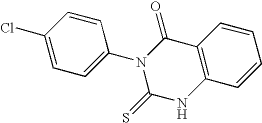 |
| 77 | 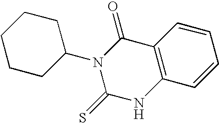 |
| 78 | 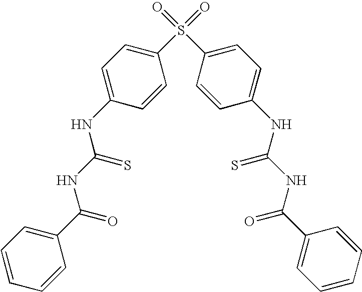 |
| 79 | 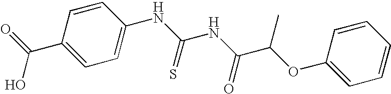 |
| 80 | 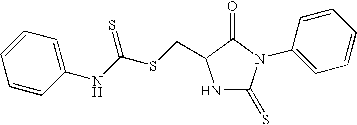 |
| 81 | 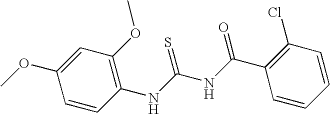 |
| 82 | 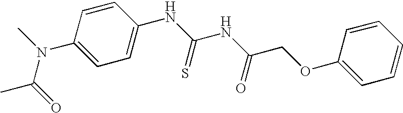 |
| 83 | 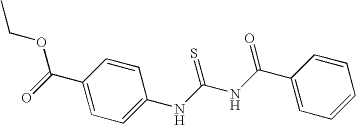 |
| 84 | 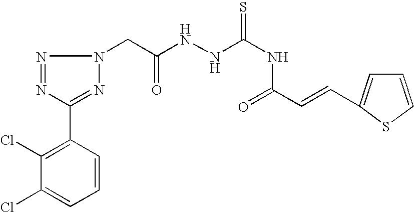 |
| 85 | 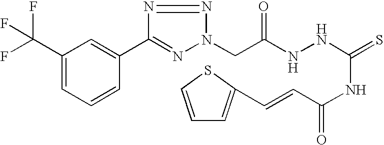 |
| 86 | 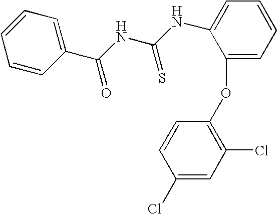 |
| 87 | 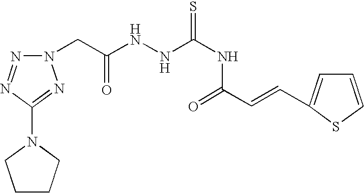 |
| 88 | 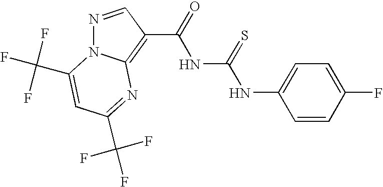 |
| 89 | 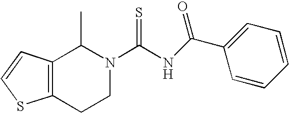 |
| 90 | 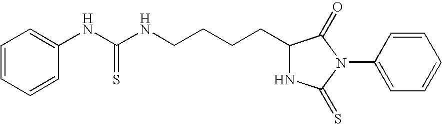 |
| 91 | 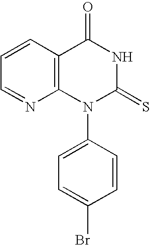 |
| 92 | 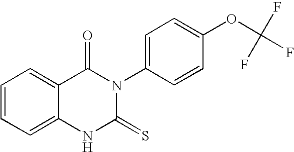 |
| 93 | 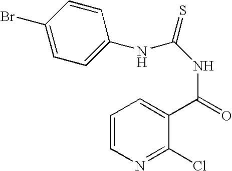 |
| 94 | 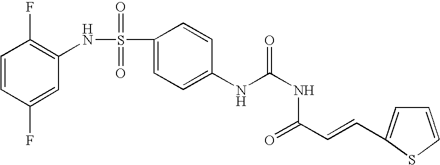 |
| 95 | 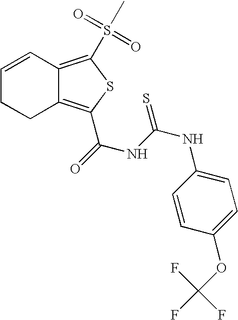 |
| 96 | 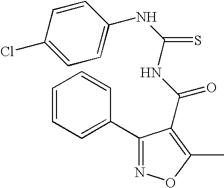 |
| 97 | 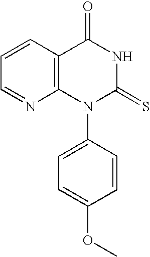 |
| 98 | 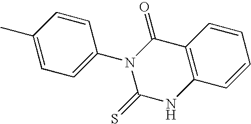 |
| 99 | 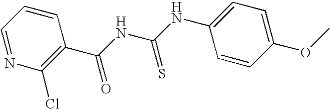 |
| 100 | 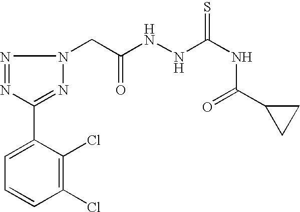 |
| 101 | 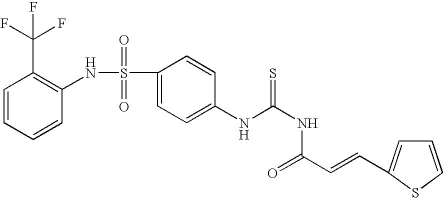 |
| 102 | 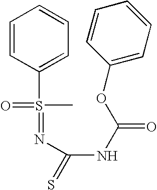 |
| 103 | 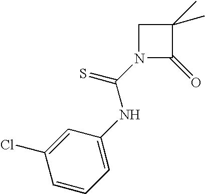 |
| 104 | 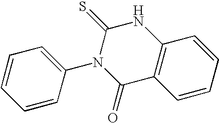 |
| 105 | 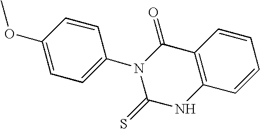 |
| 106 | 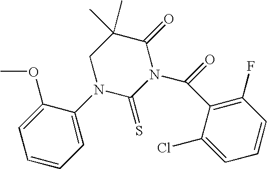 |
| 107 | 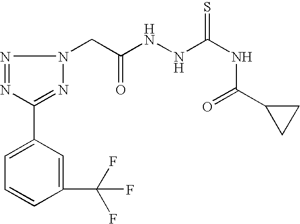 |
| 108 | 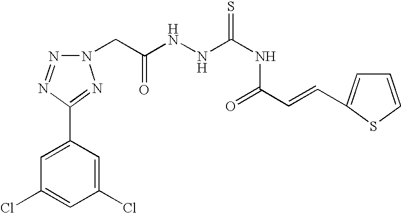 |
| 109 | 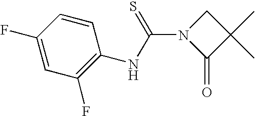 |
| 110 | 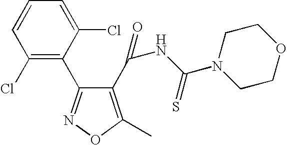 |
| 111 | 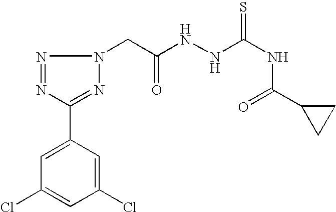 |
| 112 | 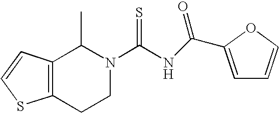 |
| 113 | 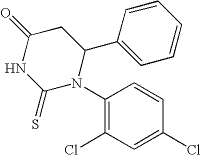 |
| 114 | 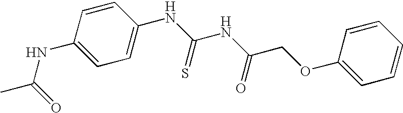 |
| 115 | 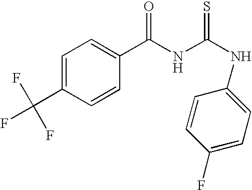 |
| 116 | 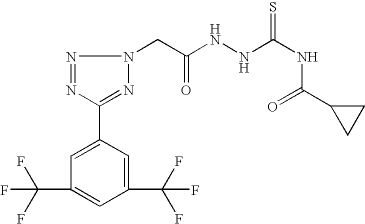 |
| 117 | 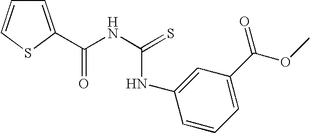 |
| 118 | 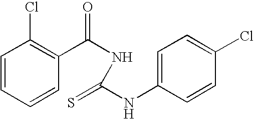 |
| 119 | 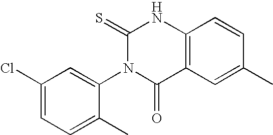 |
| 120 | 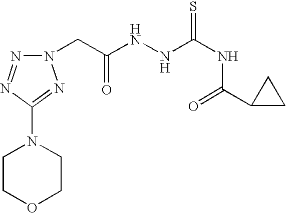 |
| 121 | 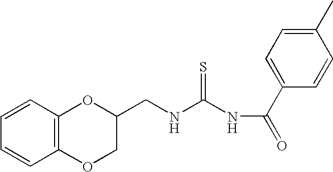 |
| 122 | 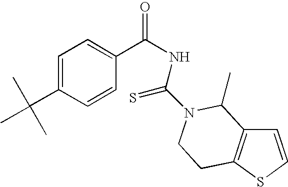 |
| 123 | 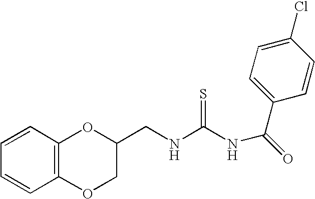 |
| 124 | 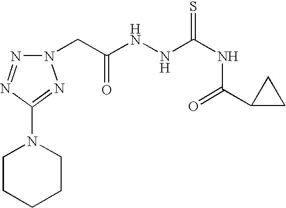 |
| 125 | 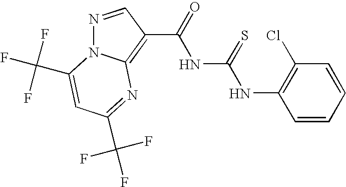 |
| 126 | 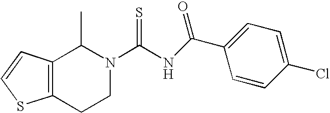 |
| 127 | 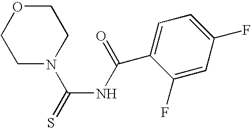 |

| | STRUCTURE |
|---|---|
| 1 | 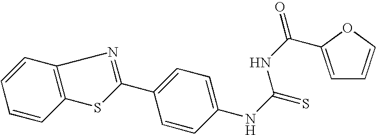 |
| 2 | 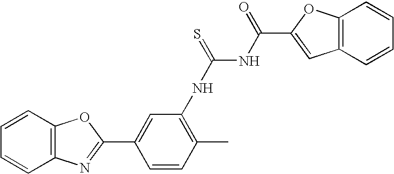 |
| 3 | 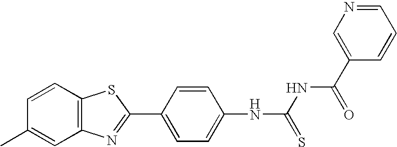 |
| 4 | 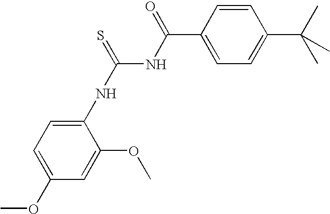 |
| 5 | 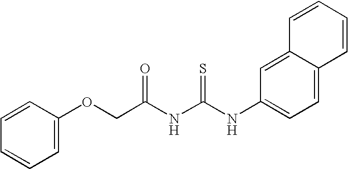 |
| 6 | 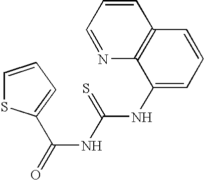 |
| 7 | 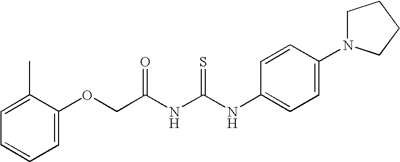 |
| 8 | 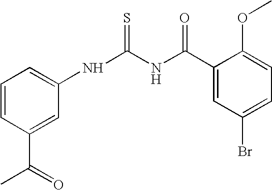 |
| 9 | 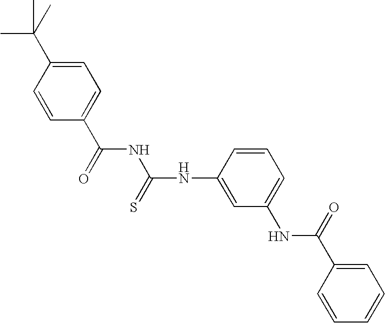 |
| 10 | 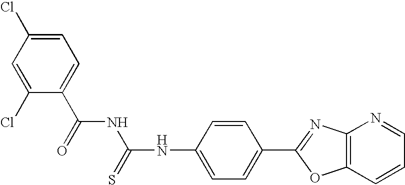 |
| 11 | 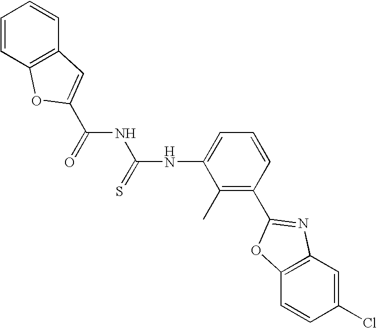 |
| 12 | 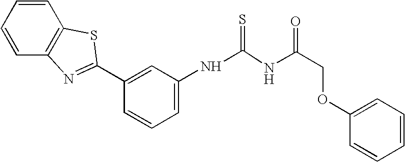 |
| 13 | 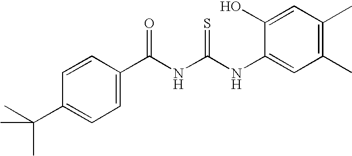 |
| 14 | 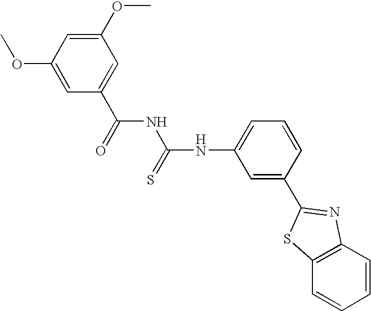 |
| 15 | 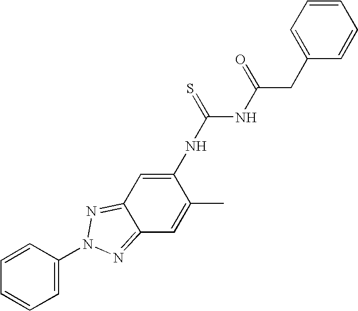 |
| 16 | 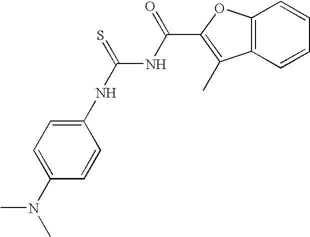 |
| 17 | 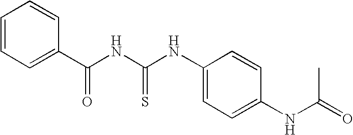 |
| 18 | 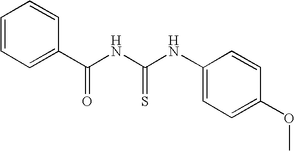 |
| 19 | 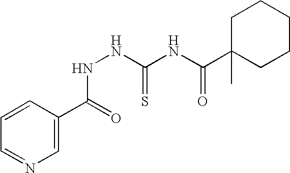 |
| 20 | 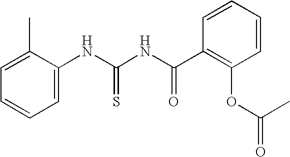 |
| 21 | 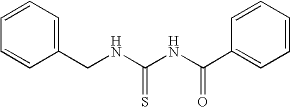 |
| 22 | 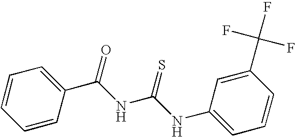 |
| 23 | 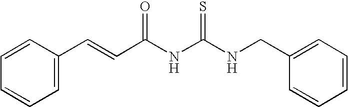 |
| 24 | 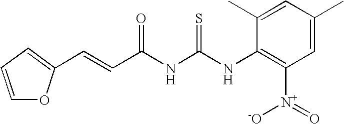 |
| 25 | 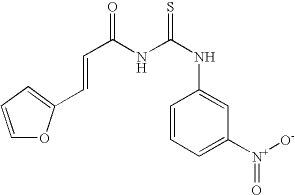 |
| 26 | 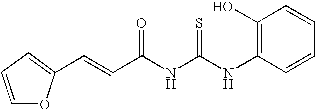 |
| 27 | 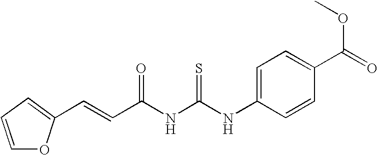 |
| 28 | 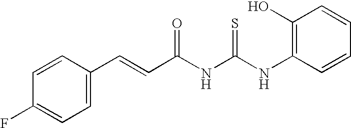 |
| 29 | 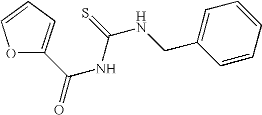 |
| 30 | 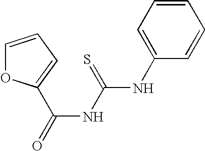 |
| 31 | 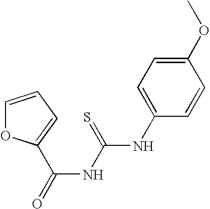 |
| 32 | 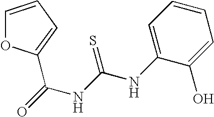 |
| 33 | 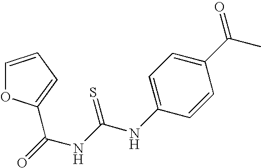 |
| 34 | 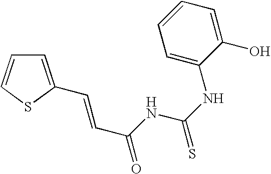 |
| 35 | 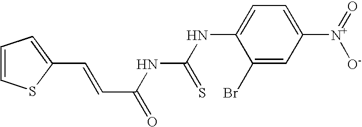 |
| 36 | 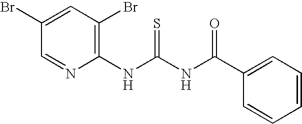 |
| 37 | 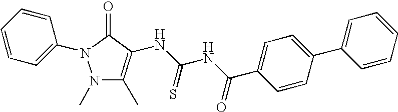 |
| 38 | 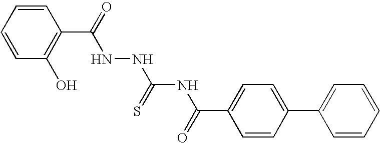 |
| 39 | 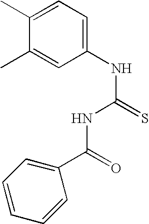 |
| 40 | 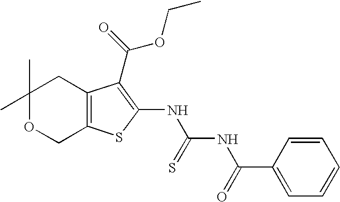 |
| 41 | 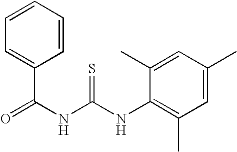 |
| 42 | 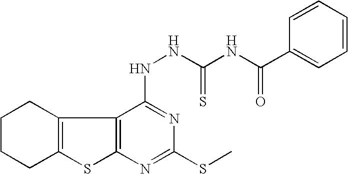 |
| 43 | 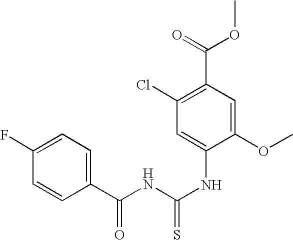 |
| 44 | 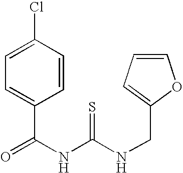 |
| 45 | 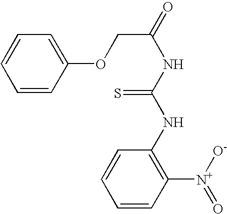 |
| 46 | 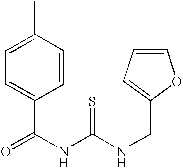 |
| 47 | 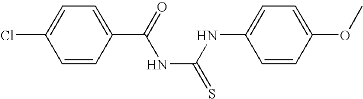 |
| 48 | 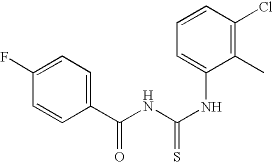 |
| 49 | 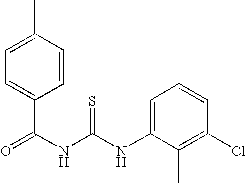 |
| 50 | 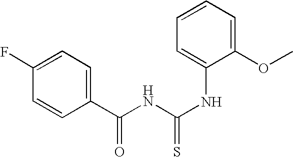 |
| 51 | 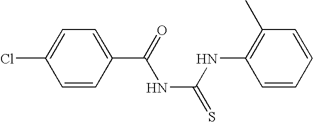 |
| 52 | 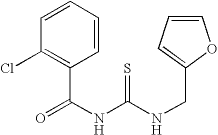 |
| 53 | 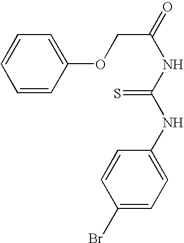 |
| 54 | 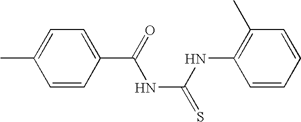 |
| 55 | 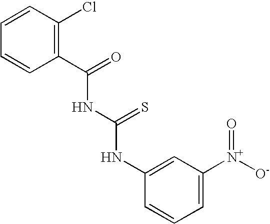 |
| 56 | 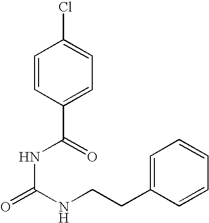 |
| 57 | 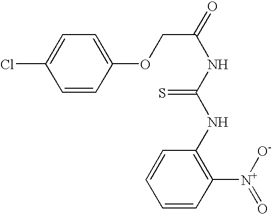 |
| 58 | 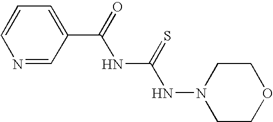 |
| 59 | 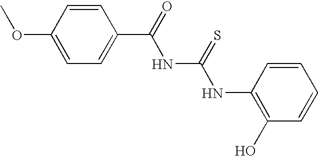 |
| 60 | 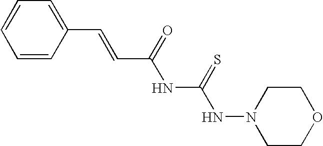 |
| 61 | 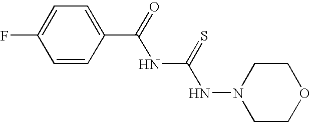 |
| 62 | 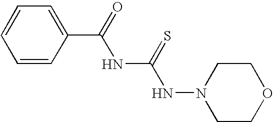 |
| 63 | 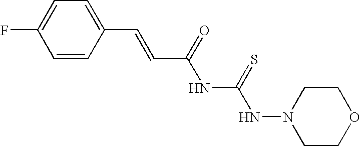 |
| 64 | 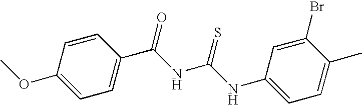 |
| 65 | 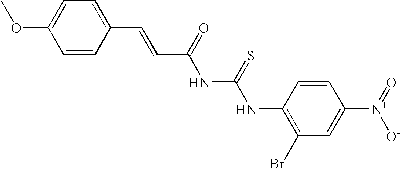 |
| 66 | 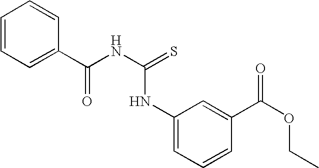 |
| 67 | 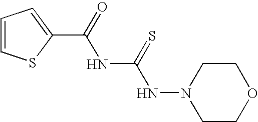 |
| 68 | 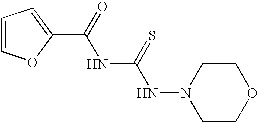 |
| 69 | 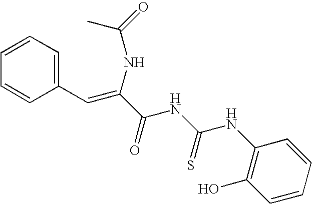 |
| 70 | 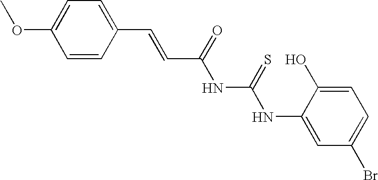 |
| 71 | 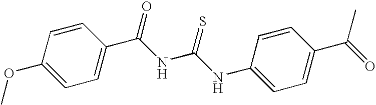 |
| 72 | 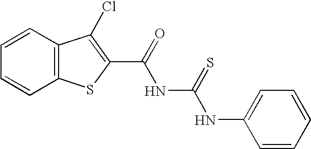 |
| 73 | 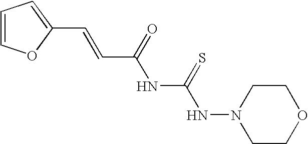 |
| 74 | 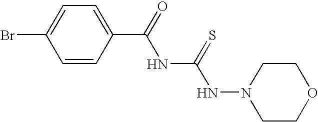 |
| 75 | 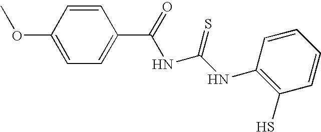 |
| 76 | 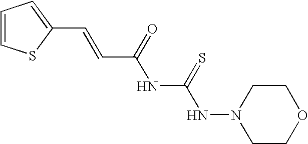 |
| 77 | 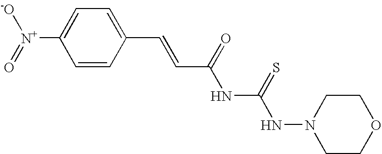 |
| 78 | 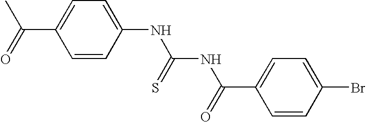 |
| 79 | 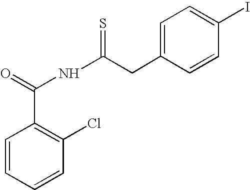 |
| 80 | 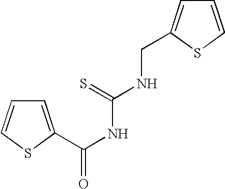 |
| 81 | 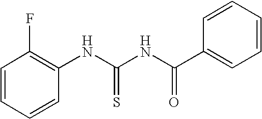 |
| 82 | 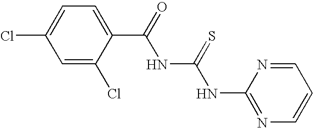 |
| 83 | 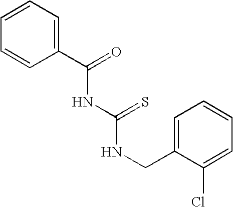 |
| 84 | 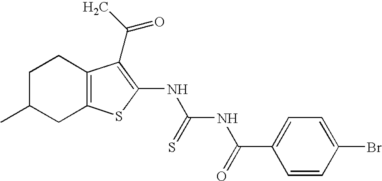 |
| 85 | 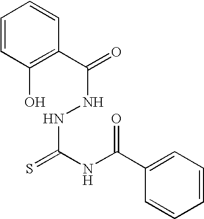 |
| 86 | 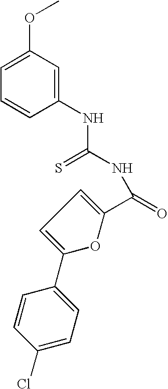 |
| 87 | 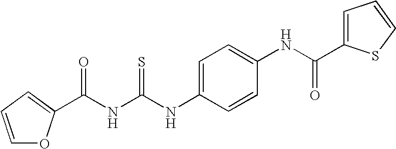 |
| 88 | 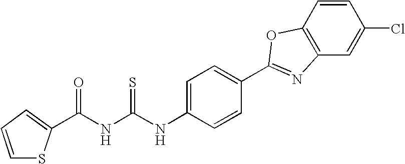 |
| 89 | 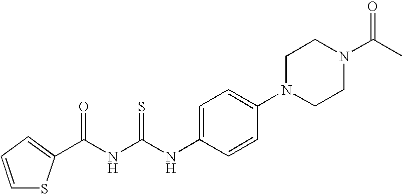 |
| 90 | 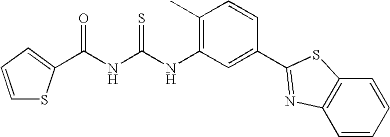 |
| 91 | 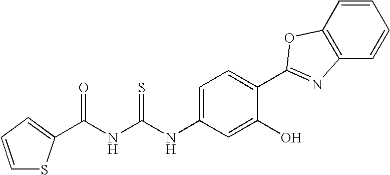 |
| 92 | 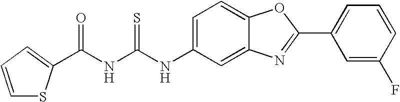 |
| 93 | 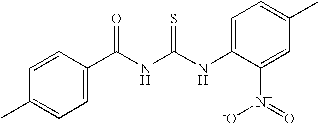 |
| 94 | 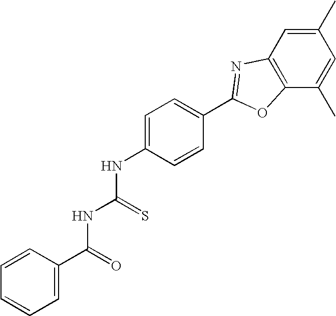 |
| 95 | 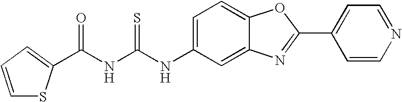 |
| 96 | 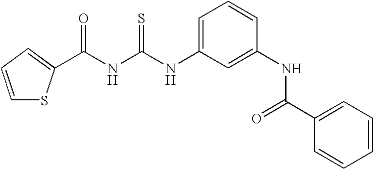 |
| 97 | 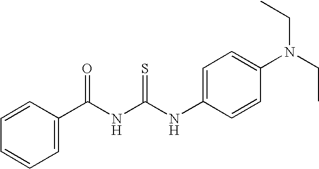 |
| 98 | 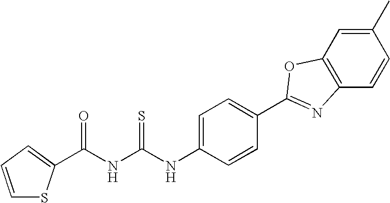 |
| 99 | 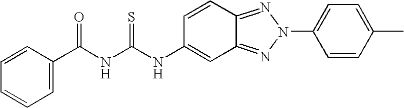 |
| 100 | 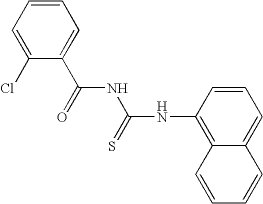 |
| 101 | 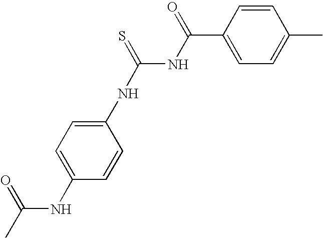 |
| 102 | 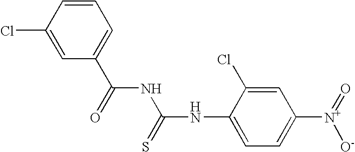 |
| 103 | 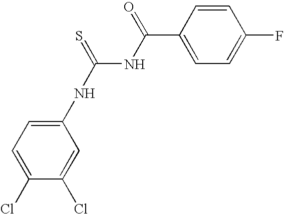 |
| 104 | 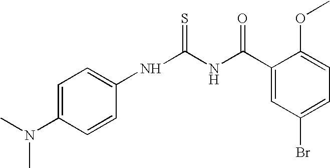 |
| 105 | 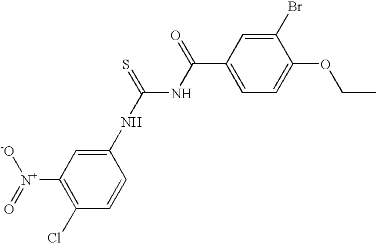 |
| 106 | 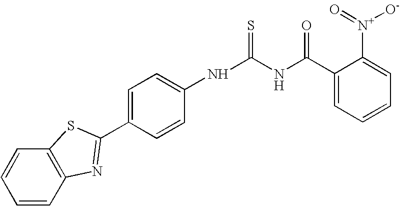 |
| 107 | 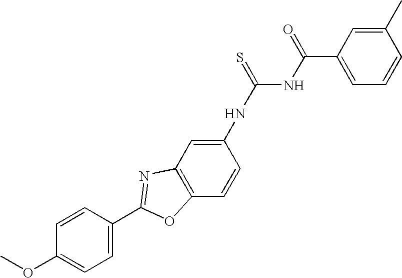 |
| 108 | 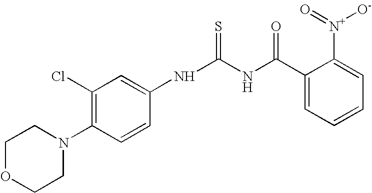 |
| 109 | 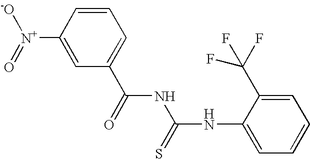 |
| 110 | 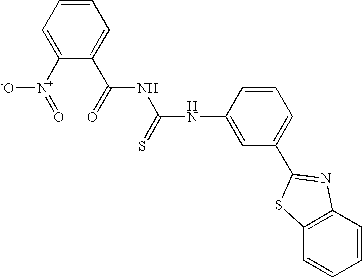 |
| 111 | 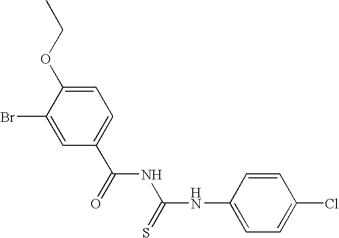 |
| 112 | 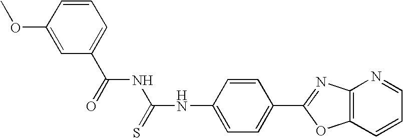 |
| 113 | 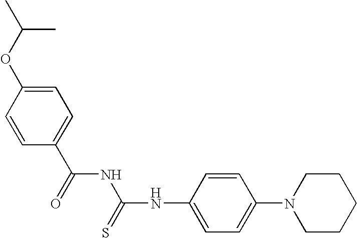 |
| 114 | 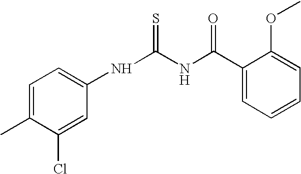 |
| 115 | 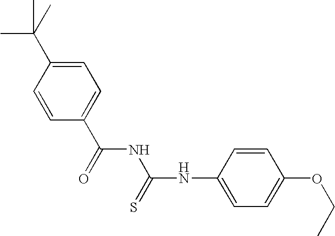 |
| 116 | 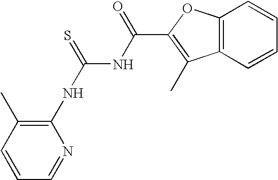 |
| 117 | 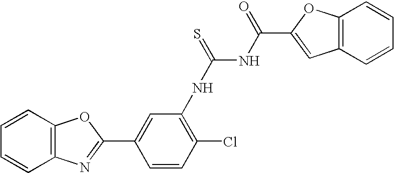 |
| 118 | 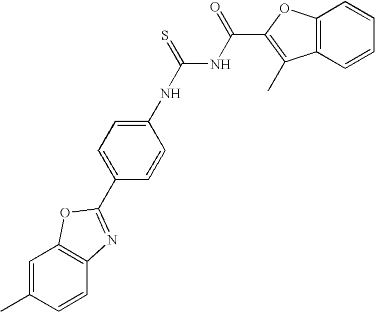 |
| 119 | 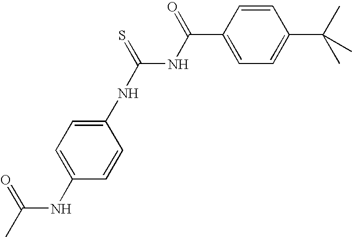 |
| 120 | 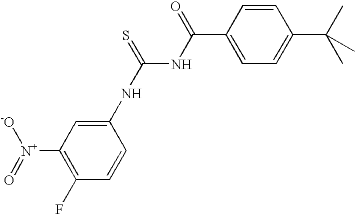 |
| 121 | 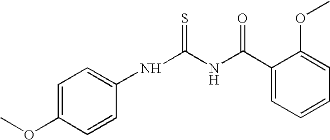 |
| 122 | 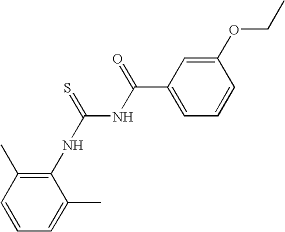 |
| 123 | 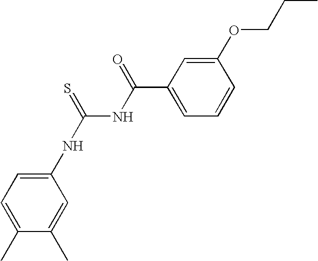 |
| 124 | 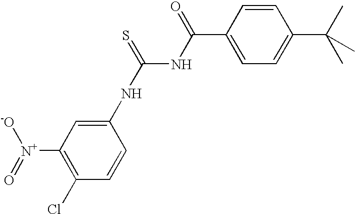 |
| 125 | 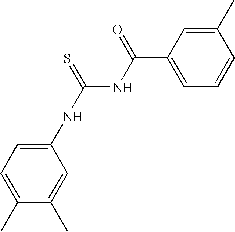 |
| 126 | 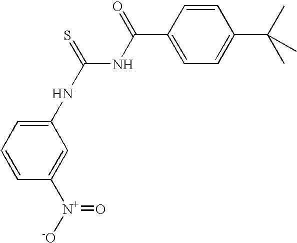 |
| 127 | 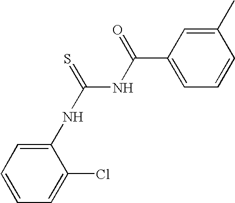 |
| 128 | 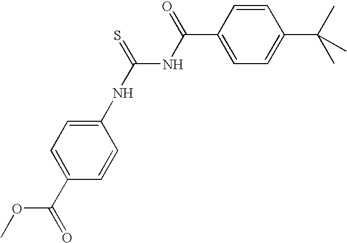 |
| 129 | 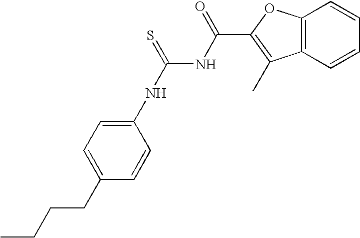 |
| 130 | 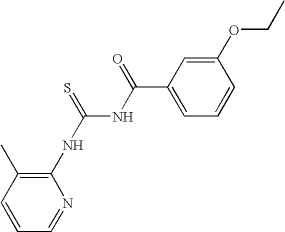 |
| 131 | 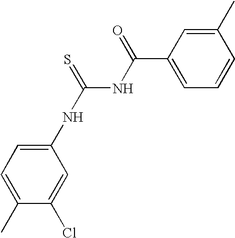 |
| 132 | 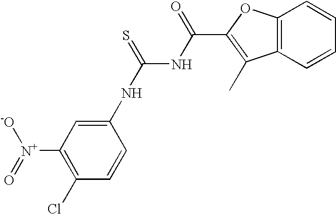 |
| 133 | 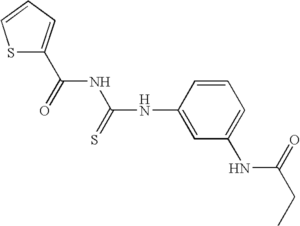 |
| 134 | 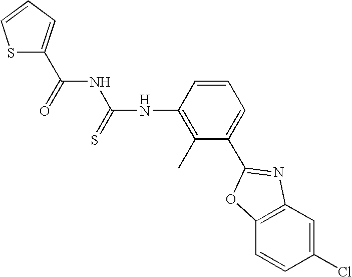 |
| 135 | 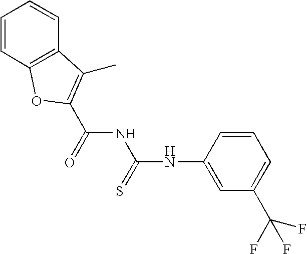 |
| 136 | 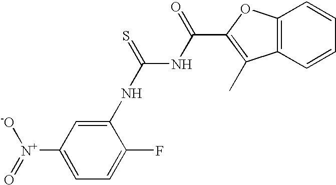 |
| 137 | 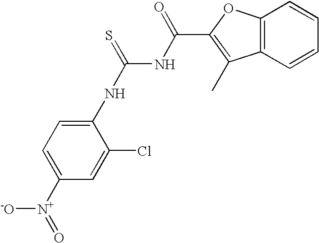 |
| 138 | 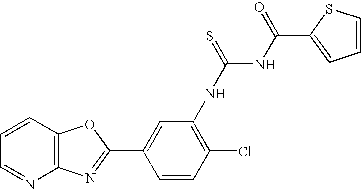 |
| 139 | 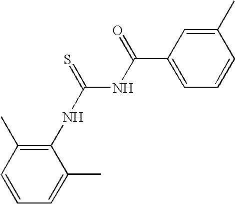 |
| 140 | 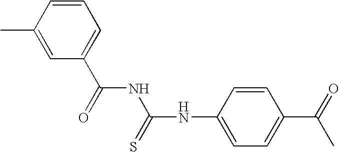 |
| 141 | 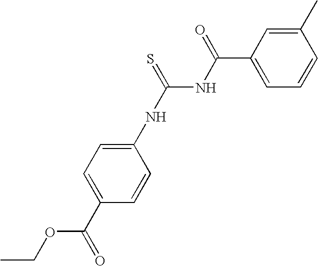 |
| 142 | 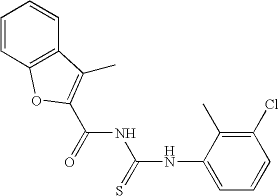 |
| 143 | 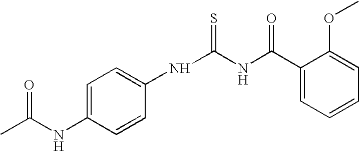 |
| 144 | 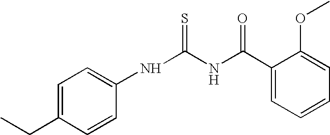 |
| 145 | 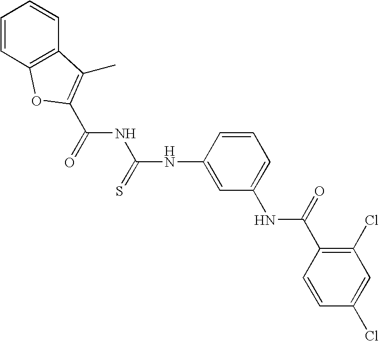 |
| 146 | 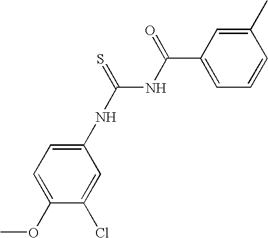 |
| 147 | 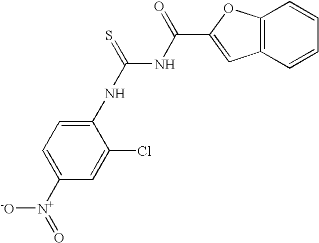 |
| 148 | 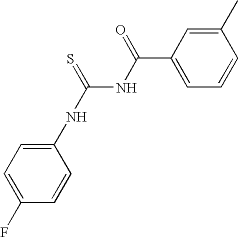 |
| 149 | 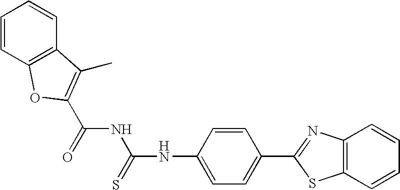 |
| 150 | 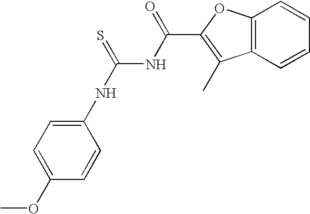 |
| 151 | 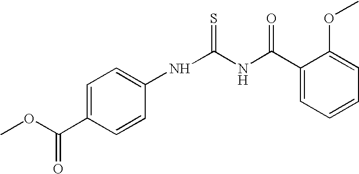 |
| 152 | 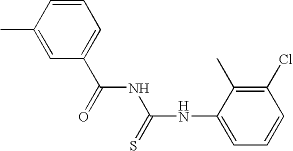 |
| 153 | 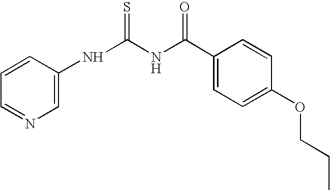 |
| 154 | 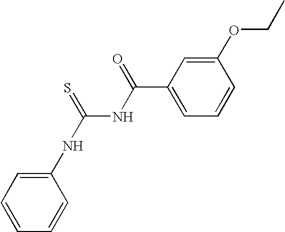 |
| 155 | 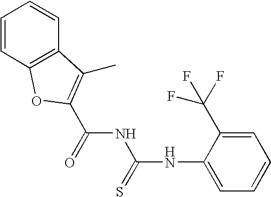 |
| 156 | 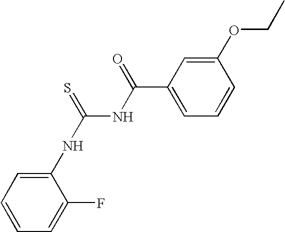 |
| 157 | 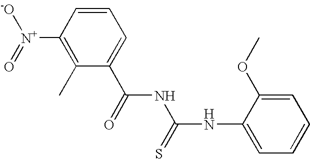 |
| 158 | 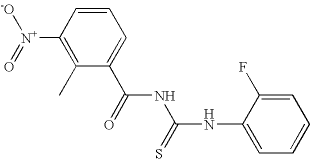 |
| 159 | 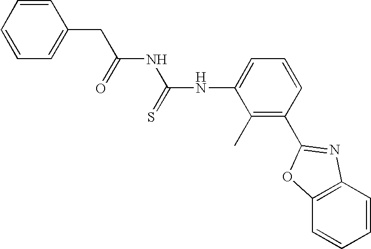 |
| 160 | 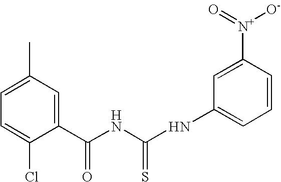 |
| 161 | 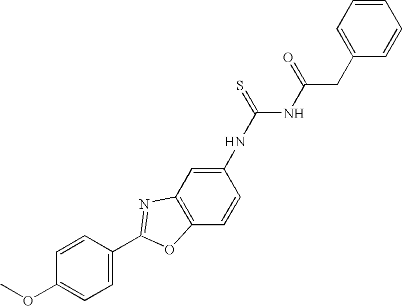 |
| 162 | 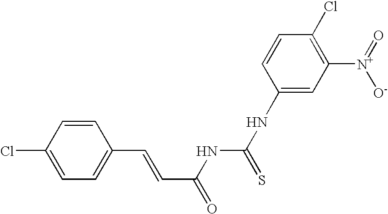 |
| 163 | 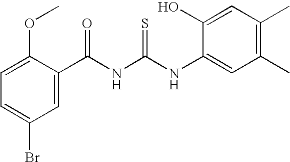 |
| 164 | 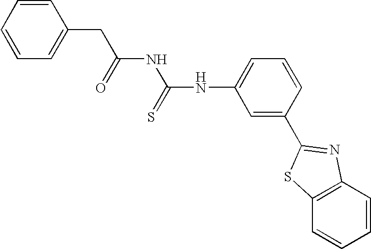 |
| 165 | 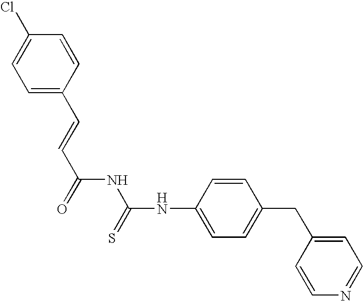 |
| 166 | 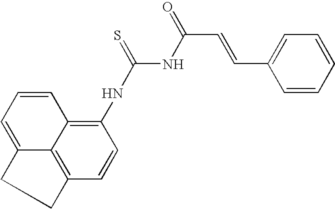 |
| 167 | 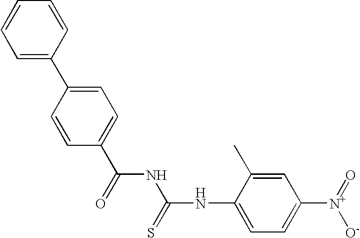 |
| 168 | 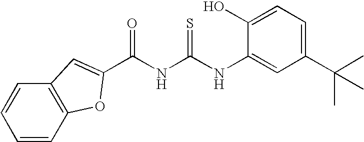 |
| 169 | 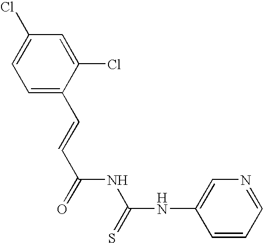 |
| 170 | 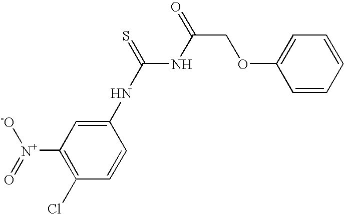 |
| 171 | 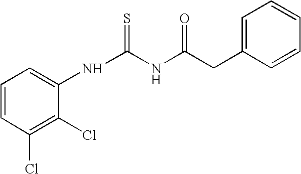 |
| 172 | 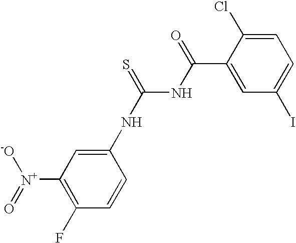 |
| 173 | 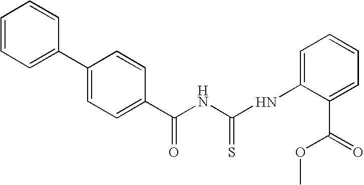 |
| 174 | 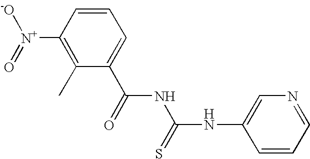 |
| 175 | 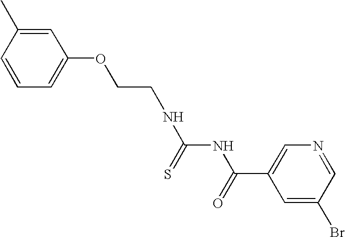 |
| 176 | 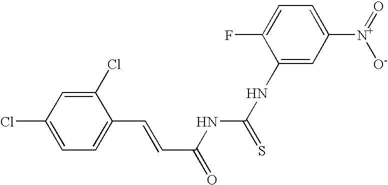 |
| 177 | 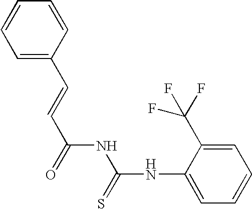 |
| 178 | 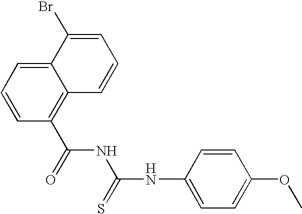 |
| 179 | 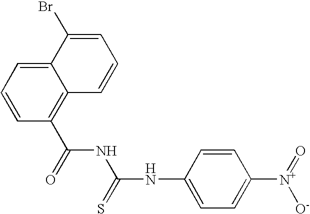 |
| 180 | 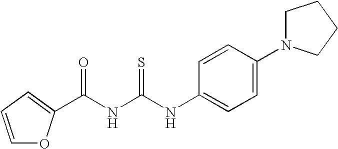 |
| 181 | 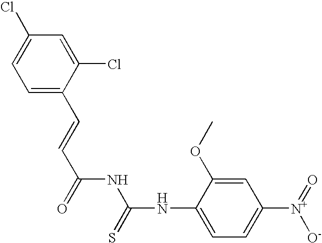 |
| 182 | 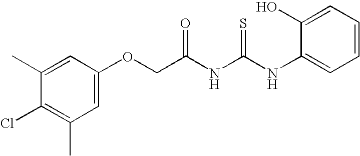 |
| 183 | 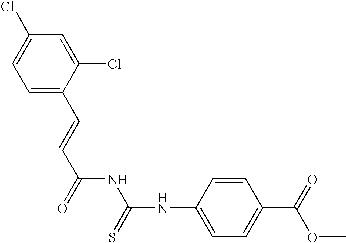 |
| 184 | 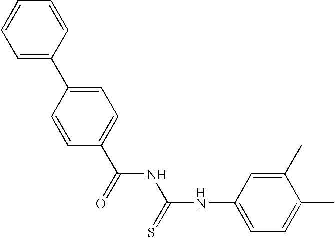 |
| 185 | 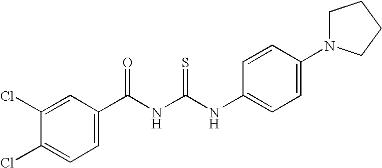 |
| 186 | 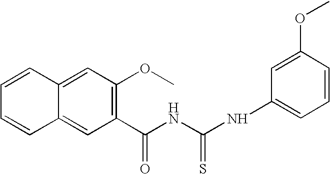 |
| 187 | 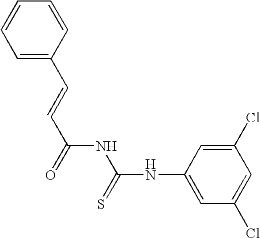 |
| 188 | 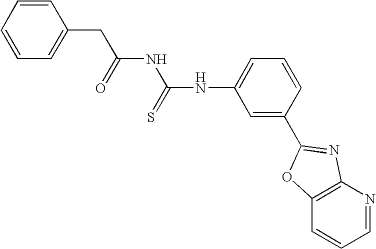 |
| 189 | 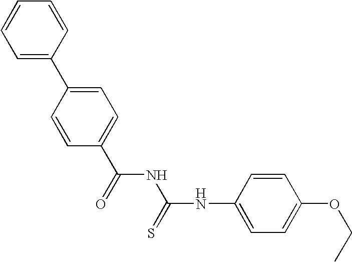 |
| 190 | 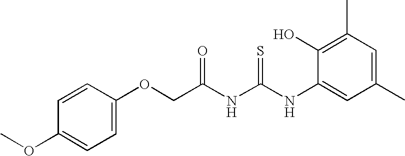 |
| 191 | 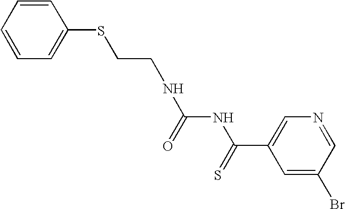 |
| 192 | 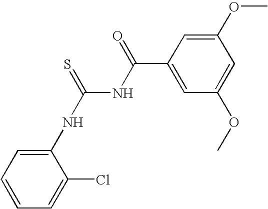 |
| 193 | 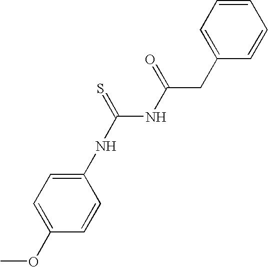 |
| 194 | 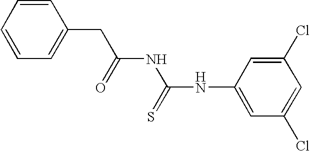 |
| 195 | 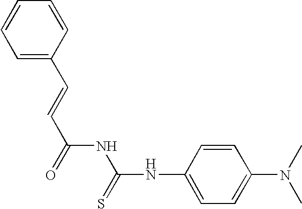 |
| 196 | 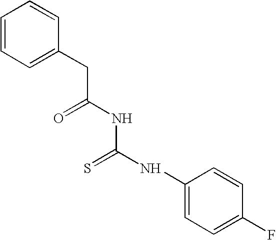 |
| 197 | 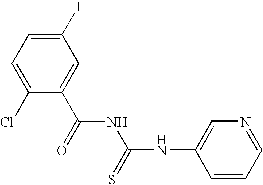 |
| 198 | 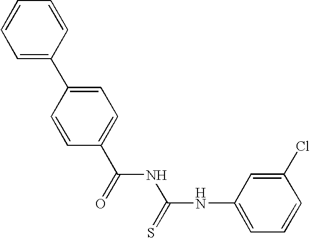 |
| 199 | 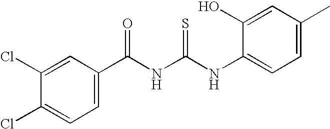 |
| 200 | 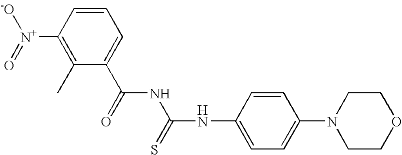 |
| 201 | 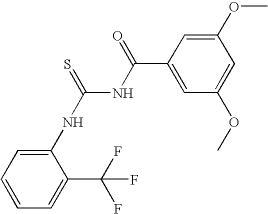 |
| 202 | 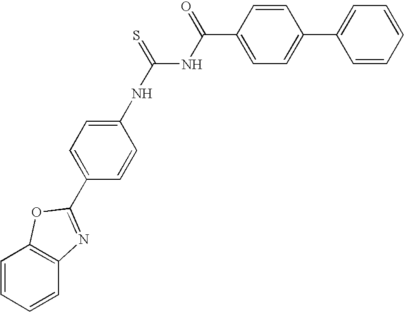 |
| 203 | 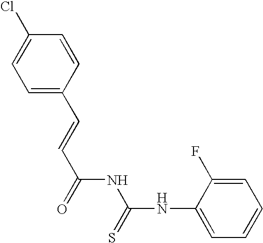 |
| 204 | 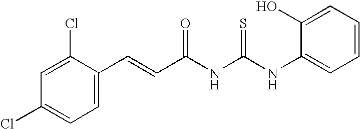 |
| 205 | 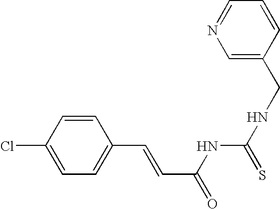 |
| 206 | 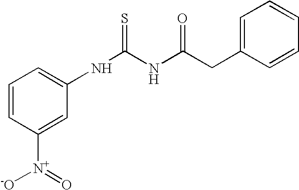 |
| 207 | 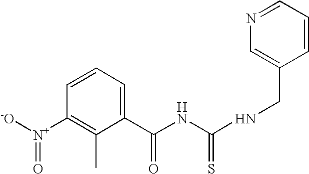 |
| 208 | 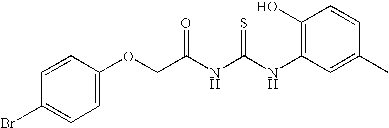 |
| 209 | 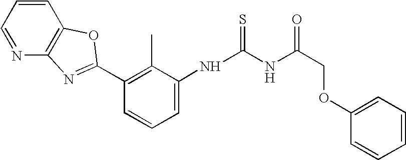 |
| 210 | 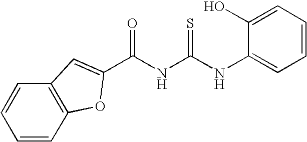 |
| 211 | 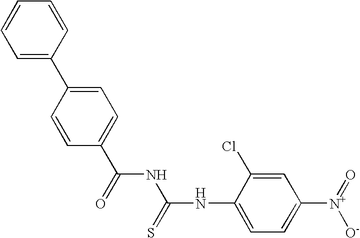 |
| 212 | 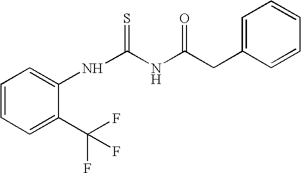 |
| 213 | 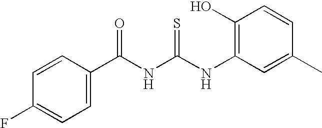 |
| 214 | 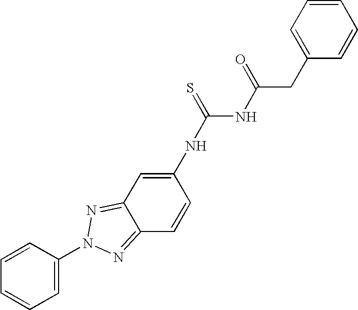 |
| 215 | 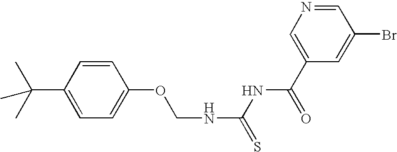 |
| 216 | 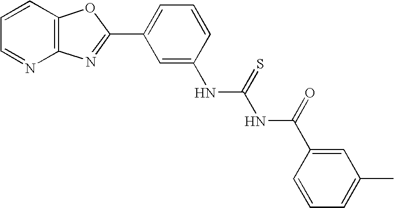 |
| 217 | 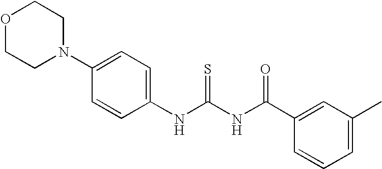 |
| 218 | 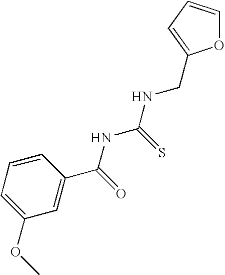 |

1. (canceled)

2. A method of identifying a mutant HCV that is resistant to a replicase complex defect inducer comprising:
growing cells that express an HCV replicon that is sensitive to the replicase complex defect inducer;
adding the replicase complex defect inducer to the cells to produce resistant cells;
amplifying the resistant cells to produce resistant colonies; and
screening the resistant colonies for the mutant HCV replicon or fragment thereof that is resistant to the replicase complex defect inducer and sensitive to an NS5B polymerase inhibitor and an NS3 protease inhibitor.

3-14. (canceled)

15. A method of screening a test compound for replicase complex defect inducer activity comprising:
providing the test compound;
contacting the test compound with mutant HCV having a mutant NS3 protein, wherein the mutant NS3 protein consists essentially of one or more mutations at or within about 15 angstroms of C16; and
identifying the test compound as an inducer of an HCV replicase complex defect when the mutant HCV is resistant to the test compound.

16-22. (canceled)

23. A method of screening a test compound for HCV replicase complex defect inducer activity comprising:
providing the test compound;
contacting the test compound with a cell expressing an HCV replicon, an isolated HCV replicase complex or an HCV protein containing NS4A; and
identifying the test compound as an inducer of an HCV replicase complex defect when a dose-dependent level of a p14 protein is increased,
wherein the p14 protein is a novel species that is present in a replicase complex that is untreated with the test compound, and wherein the p14 protein reacts with an anti-NS4A antibody.

24-27. (canceled)

28. The method of claim 2, wherein the cells that express an HCV replicon that is sensitive to a replicase complex defect inducer are in the form of an HCV virus, cells expressing a recombinant HCV replicon, or cells expressing an HCV polyprotein.

29. The method of claim 2, wherein screening further comprises
determining that an individual resistant HCV replicon is sensitive to an NS5B polymerase inhibitor and an NS3 protease inhibitor, and identifying the resistant HCV replicon as a mutant clone resistant to a replicase complex defect inducer.

30. The method of claim 2, further comprising
genotyping the mutant HCV replicon comprised by the mutant resistant to a replicase complex defect inducer; and
positively identifying a mutant that is sensitive to a replicase complex defect inducer.

31. The method of claim 30, wherein genotyping comprises:
sequencing and identifying a mutation in the mutant HCV replicon.

32. The method of claim 2, wherein the HCV replicon that is sensitive to a replicase complex defect inducer comprises an HCV subgenomic replicon selected from the group consisting of SEQ ID NO: 8, 9, 10 and 11.

33. The method of claim 15, wherein the mutant NS3 protein has a mutation in position 16 or position 39.

34. The method of claim 33, wherein the mutation is C16S or A39V.

35. The method of claim 15, further comprising identifying the test compound as a compound that does not block NS5B polymerase activity.

36. The method of claim 15, wherein identifying the test compound as a compound that does not block NS5B polymerase activity comprises demonstrating that the compound does not block the activity of pre-formed replicase complexes.

37. The method of claim 15, further comprising identifying the test compound as a compound that inhibits the production of functional replicase complexes by selectively reducing the amount of NS3 and NS4A.

38. The method of claim 15, further comprising identifying the test compound as a compound that inhibits the production of functional replicase complexes by selectively inducing the formation of p14, wherein the p14 protein is a novel species that is present in a replicase complex that is untreated with the test compound, and wherein the p14 protein reacts with an anti-NS4A antibody.

39. The method of claim 23, wherein the dose-dependent increase in the p14 protein is accompanied by a dose-dependent decrease in the level of NS3 and NS4A.

40. The method of claim 23, wherein identifying comprises detecting the p14 protein with the anti-NS4A antibody.

41. The method of claim 23, wherein identifying further comprises identifying the replicase complex defect inducer as a compound that selectively binds NS4A.

42. A method of treating an individual in need of treatment for HCV comprising administering a therapeutically effective amount of an inducer of an HCV replicase complex defect identified by the method of claim 15.

43. A method of treating an individual in need of treatment for HCV comprising administering a therapeutically effective amount of an inducer of an HCV replicase complex defect identified by the method of claim 23.

44. An inducer of an HCV replicase complex defect for which a mutant HCV having a mutant NS3 protein, which is resistant to the inducer compound,
wherein the mutant NS3 protein consists essentially of one or more mutations at or within about 15 angstroms of C16, wherein the inducer is a compound of the formula 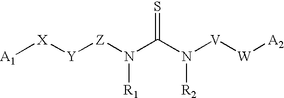
or a pharmaceutically acceptable salt thereof, wherein:
A₁ and A₂ are independently optionally substituted C₁-C₁₂alkyl, optionally substituted mono- or di-(C₁-C₈alkyl)amino, optionally substituted C₂-C₁₂alkenyl, optionally substituted C₃-C₈cycloalkyl, a partially unsaturated or aromatic carbocyclic group, or an optionally substituted saturated, partially unsaturated, or aromatic heterocyclic group; wherein at least one of A₁ and A₂ is an optionally substituted aromatic carbocyclic group or an optionally substituted aromatic heterocyclic group;
X and W are independently O, S, NR, or absent, where R is hydrogen, optionally substituted C₁-C₆alkyl, or optionally substituted aryl(C₀-C₄alkyl);
V is C₁-C₆ alkyl, C₂-C₆alkenyl, C₃-C₇cycloalkyl, or absent; and Y is C₁-C₆ alkyl, C₁-C₆ alkyl substituted with C₃-C₇cycloalkyl, C₂-C₆alkenyl, C₃-C₇cycloalkyl, or absent; wherein when V is absent, W is absent; and Z is carbonyl, thiocarbonyl, imino, or C₁-C₆alkylimino; and
R₁ and R₂ are independently hydrogen or
R₁ and R₂ are independently C₁-C₆alkyl, C₂-C₆ alkenyl, or C₂-C₆ alkynyl, each of which is substituted with 0 to 3 substituents independently chosen from halogen, hydroxy, amino, C₁-C₄alkoxy, C₁-C₂haloalkyl, and C₁-C₂haloalkoxy, or
R₁ and R₂ are joined to form a 5- to 7-membered saturated or mono-unsaturated ring optionally containing one additional heteroatom chosen from N, S, and O, which 5- to 7-membered saturated or mono-unsaturated ring is substituted with 0 to 3 substituents independently chosen from halogen, hydroxy, amino, C₁-C₄alkyl, C₁-C₄alkoxy, mono- and di-(C₁-C₄alkyl)amino, C₁-C₂haloalkyl, and C₁-C₂haloalkoxy;
wherein the inducer is not a compound of Appendix A, B, or C.

45. An inducer of an HCV replicase complex defect, for which a dose dependent increase in level of p14 protein is observed when the inducer is contacted with a cell expressing an HCV replicon, an isolated HCV replicase complex or an HCV protein containing NS4A;
wherein the inducer is a compound of the formula 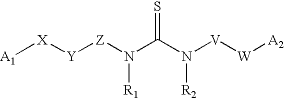
or a pharmaceutically acceptable salt thereof, wherein:
A₁ and A₂ are independently optionally substituted C₁-C₁₂alkyl, optionally substituted mono- or di-(C₁-C₈alkyl)amino, optionally substituted C₂-C₁₂alkenyl, optionally substituted C₃-C₈cycloalkyl, a partially unsaturated or aromatic carbocyclic group, or an optionally substituted saturated, partially unsaturated, or aromatic heterocyclic group; wherein at least one of A₁ and A₂ is an optionally substituted aromatic carbocyclic group or an optionally substituted aromatic heterocyclic group;
X and W are independently O, S, NR, or absent, where R is hydrogen, optionally substituted C₁-C₆alkyl, or optionally substituted aryl(C₀-C₄alkyl);
V is C₁-C₆ alkyl, C₂-C₆alkenyl, C₃-C₇cycloalkyl, or absent; and Y is C₁-C₆ alkyl, C₁-C₆ alkyl substituted with C₃-C₇cycloalkyl, C₂-C₆alkenyl, C₃-C₇cycloalkyl, or absent; wherein
when V is absent, W is absent; and Z is carbonyl, thiocarbonyl, imino, or C₁-C₆alkylimino; and
R₁ and R₂ are independently hydrogen or
R₁ and R₂ are independently C₁-C₆alkyl, C₂-C₆ alkenyl, or C₂-C₆ alkynyl, each of which is substituted with 0 to 3 substituents independently chosen from halogen, hydroxy, amino, C₁-C₄alkoxy, C₁-C₂haloalkyl, and C₁-C₂haloalkoxy, or
R₁ and R₂ are joined to form a 5- to 7-membered saturated or mono-unsaturated ring optionally containing one additional heteroatom chosen from N, S, and O, which 5- to 7-membered saturated or mono-unsaturated ring is substituted with 0 to 3 substituents independently chosen from halogen, hydroxy, amino, C₁-C₄alkyl, C₁-C₄alkoxy, mono- and di-(C₁-C₄alkyl)amino, C₁-C₂haloalkyl, and C₁-C₂haloalkoxy.
wherein the inducer is not a compound of Appendix A, B, or C.
